(19) United States
(12) Patent Application Publication
(10) Pub. No.: US 2022/0289732 A1
(43) Pub. Date: Sep. 15, 2022

(54) HETEROCYCLIC WDR5 INHIBITORS AS ANTI-CANCER COMPOUNDS

(71) Applicant: NOVARTIS AG, Basel (CH)

(72) Inventors: Lichao FANG, Shanghai (CN); Zhenting GAO, Shanghai (CN); Xiangqing JIANG, Shanghai (CN); Kevin Kun Chin LIU, Shanghai (CN); Sing Yeung Frankie MAK, Singapore (SG); Counde OYANG, Sunnyvale, CA (US); Ce WANG, Beijing (CN); Tao WANG, Sudbury, MA (US); Jianping WU, Shanghai (CN); Wu YINGMING, Shanghai (CN); Qitao XIAO, Shanghai (CN)

(21) Appl. No.: 17/633,495

(22) PCT Filed: Aug. 7, 2020
(86) PCT No.: PCT/IB2020/057484

§ 371 Date: Feb. 7, 2022

(30) Foreign Application Priority Data
Aug. 9, 2019 (CN) PCT/CN2019/099893

(51) Int. Cl. C07D 413/14; C07D 401/14; C07D 405/14; C07D 491/107; C07D 401/06; C07D 498/04; C07D 471/04; C07D 401/04

(52) U.S. Cl. CPC C07D 413/14; C07D 401/14; C07D 405/14; C07D 491/107; C07D 401/06; C07D 498/04; C07D 471/04; C07D 401/04

(57) ABSTRACT

The present invention provides compounds of Formula (I) or a pharmaceutically acceptable salt thereof; (I) which are inhibitors of WDR5. The present invention also provides pharmaceutical compositions comprising such compounds, compositions comprising such compounds with an additional therapeutic agent and the therapeutic uses of such compounds.

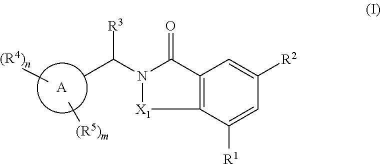

### FIELD OF THE INVENTION

The field of the invention relates to inhibitors of WD Repeat Domain 5 (WDR5) and the treatment of diseases associated with WDR5.

### BACKGROUND OF THE INVENTION

The preceding decade of biomedical research has resulted in new understandings and insight into many of the chemical modifications of histone proteins that are critical for gene regulation. As an example of relevance here, it is now well appreciated that the histone proteins exhibit specific posttranslational modifications with rich information content. These histone modifications form a sort of “histone code” that plays a central role in regulating gene expression. As insight grows into these epigenetic mechanisms, it is having a profound impact on translational biology. Recent data suggests that epigenetic changes in normal tissue may precede and predispose one to cancer, similar to the way that altered lipid metabolism may predispose one to heart disease decades before it becomes symptomatic. For other diseases there is a growing consensus that epigenetic changes (allele-specific silencing, methylation, chromatin modification) play a major role. Heritable epigenetic traits appear to play a role in neuropsychiatric disease, bipolar disease, and autism.

The development of drugs that modify histone markers has largely targeted the histone acetyltransferases and histone deacetylases. These enzymes add or remove acetyl groups on specific conserved lysine amino acids in certain histone proteins. This research has resulted in new pharmaceuticals such as the histone deacetylase inhibitor Vorinostat, marketed under the trade name ZOLINA for treating certain cancers. Acetylation and methylation are two of the most prominent posttranslational modifications of the histones that control gene expression. Therefore, active agents that can target histone methyltransferases and histone demethylases provide an important next avenue in drug development.

More specifically, abnormalities in H3-K4 methylating enzymes have been observed in various cancers (Huntsman, D. G. et al. Oncogene 1999, 18, 7975-7984; Ruault, M. et al. Gene 2002, 284, 73-81). These are multi-subunit complexes of several proteins, including WD Repeat Domain 5 (WDR5), Absent Small or Homeotic-Like (Ash2L), and Retinoblastoma Binding Protein 5 (RbBP5), each of which is a common component of all known human H3-K4 methylating complexes.

WDR5 forms a catalytically active core complex with MLL, RbBP5, and Ash2L that can dimethylate H3-K4 in vitro (Patel, A. et al. J Biol Chem 2008, 283, 32162-32175). All of the members of the core complex are required for dimethylation, including WDR5, which forms a bridge between MLL and the remainder of the core complex. In the absence of WDR5, MLL is unable to associate with RbBP5 and Ash2L, and fails to dimethylate H3-K4 in vitro (Patel, A. et al. J Biol Chem 2008, 283, 32162-32175; Dou, Y. et al. Nat Struct Mol Biol 2006, 13, 713-719). Knock-down of WDR5 is known to result in a significant decrease in the levels of H3-K4 trimethylation and expression of Hox-a9 and Hox-c8 genes in 293 cells (Wysocka, J. et al. Cell 2005, 121, 859-872). WDR5 has a canonical conformation that contains a central cavity, and both H3 and MLL peptides use an Arg residue to interact with this cavity through the arginine binding site.

Heterocyclic compounds acting as inhibitors have been reported as therapeutics for treating cancer or other disorders (WO 9009997, WO 09092566, WO 9626187, WO 0172712, WO 07149031, WO 08073305, WO 08073306, WO 10077947, WO 11088192, WO 11133728, WO 12017020, WO 12028300, WO 12154760, WO 13009827, WO 15066188, WO 14182829, and WO 15073308) Because of their therapeutic value, new inhibitors of WDR5 are needed to treat disorders associated with undesired levels of WDR5 activity. The current invention provides novel compounds that inhibit WDR5, for use to treat diseases such as cancer that are associated with excessive activity of WDR5.

Chromosomal rearrangements of the human MLL1/KMT2A gene are associated with infant, pediatric, adult and therapy-induced acute leukemias, which is also called Mixed Lineage Leukemia (MLL or MLL-r) that presents a heterogeneous group of AML (acute myeloid leukemia) and ALL (acute lymphoblastic leukemia) bearing features (Pui C. H., et al. Lancet 2002; 359: 1909-1915, Pui C. H., et al. Leukemia 2003; 17: 700-706). MLL patients have poor prognosis with overall 5-year survival rate around 35% (Dimartino J F, et al. Br J Haematol. 1999, 106, 614-626; Tomizawa, D.; et. al. Leukemia, 2007, 21, 2258-2263). In MLL1 rearranged leukemia, reciprocal translocation of MLL1 gene results in in-frame fusion of the 5′-end MLL1 with the 3′-end of the fusion partner gene (Tkachuk, D. C., et al. Cell 1992, 71, 691-700). MLL1 fusion proteins (MLL-FPs) together with its aberrantly recruited factors likely transform cells through altering gene expression such as homeobox (Hox) genes (Guenther, M. G. et al. Proc Natl Acad Sci USA 2005, 102, 8603-8608; Slany, R. K. Haematologica, 2009, 94, 984-993). A common feature of MLL1 abnormality in MLL-r leukemia is the preservation of one wild-type MLL1 allele (Thiel, A. T. et al. Cancer Cell 2010, 17, 148-159).

MLL1 gene locates on chromosome 11q23 and the encoded MLL1 protein is a homology of Drosophila trithorax (Trx) (Tkachuk, D. C., et al. Cell 1992, 71, 691-700). Wild-type MLL1 binds to its target genes through its N-terminal gene recognition elements while the catalytic C-terminal SET domain catalyzes histone 3 lysine 4 (H3K4) which methylation is predominantly associated with transcriptionally active genes (Hsieh, J. J. D. et al. Mol. Cell Biol. 2003, 23, 186-194; Shilatifard, A. Curn Opin Cell Biol. 2008, 20, 341-348). MLL1 is only highly enzymatically active in the multiprotein complex. Intrinsic histone methyltransferase (HMT) activity of MLL1 is extremely low and requires the formation of a core complex including WD Repeat Domain 5 (WDR5), Absent Small or Homeotic-Like (Ash2L), and Retinoblastoma Binding Protein 5 (RbBP5), each of which is a common component of all known human H3K4 methylating complexes in KMT2 family (Dou, Y. et al. Nature Struct. Mol. Biol. 2006, 13, 713-719).

WDR5 stably bridges RBBP5 and MLL1 via direct binding to a conserved WDR5-interacting motif in MLL1 (Patel, A., et al. J. Biol. Chem. 2008, 283, 32158-32161; 13) and a Val-Asp-Val motif in RBBP5 (Avdic, V. et al. Structure 2011, 19, 101-108). In the absence of WDR5, MLL is unable to associate with RbBP5 and Ash2L and fails to methylate H3K4 in vitro (Patel, A. et al. J. Biol. Chem. 2008, 283, 32162-32175). Despite conservation of the core entity, WDR5 is only necessary in regulating MLL1 while not other KMT2 complexes (Southall, S. M., et al. Mol. Cell 2009, 33, 181-191). MLL1 binds to WDR5 via an arginine (Arg) containing sequence (WIN motif) (Patel, A., et al. J. Biol. Chem. 2008, 283, 32158-32161; Song, J. J. & Kingston, R. E. J. Biol. Chem. 2008, 283, 35258-35264), and disruption of WDR5-MLL1 binding by this WDR5-interacting peptides (or their derivatives) leads to disintegration of the MLL1 complex and inhibition of MLL1 catalytic activity in vitro (Patel, A. et al. J. Biol. Chem. 2008, 283, 32162-32175; Dharmarajan, V. et al. J. Biol. Chem. 2012, 287, 27275-27289; Karatas, H. et al. J. Med. Chem. 2010, 53, 5179-5185).

It has been reported that MLL1-AF9 induced leukemogenesis requires co-expression of the wild type MLL1 allele since genetic deletion of MLL1 in MLL1-AF9 murine leukemia cells reduced clonogenic potential and leukemia progression. Also peptidomimetics have been discovered that bind tightly to WDR5 at the MLL1 binding site, it can inhibit MLL1 methyltransferase activity and block proliferation of MLL-r cells by inducing cell-cycle arrest, apoptosis and myeloid differentiation (Cao, F. et al. Mol. Cell 2014, 53, 247-261). Thus, interruption of the WDR5-MLL1 protein-protein interaction (PPI) may be a useful strategy for treating patients with MLL-r leukemia.

As an highly conserved WD40 repeat-containing protein that is essential for proper regulation of multiple cellular processes, WDR5 acts as a scaffold protein interacting with multiple proteins or protein complexes including histone H3, MOF, C/EBPα, Myc and the NuRD complex (Song, J. J. & Kingston, R. E. J. Biol. Chem. 2008, 283, 35258-35264; Dou, Y. et al. Cell 2005, 121, 873-885; Dias, J., et al. Genes & Development 2014, 28, 929-942; Senisterra, G., et al. Biochem. J., 2013. 449, 151-159; Thomas, L. R.; et al. 2015, 58, 440-452; Ee, L.-S., et al. Stem Cell Reports, 2017, 8, 1488-1496) in addition to the well characterized partner MLL1. Disruption of WDR5 with these proteins could have desirable benefit as cancer treatment strategies considering many of them are important transcription factors or modulators. Moreover, WDR5 expression levels have been reported to be deregulated and could be correlative to patient prognosis in several cancer types including neuroblastoma, breast cancer, bladder cancer colorectal cancer and Papillary Thyroid Carcinoma (Sun, Y. et al. Cancer Research, 2015, 75, 5143-5154; Dai, X. et al. PLoSOne, 2015, 10, PMC4565643; Chen, X. et al. Scientific Reports, 2015, 5, 8293; Tan, X et al. Cell Death & Disease, 2017, 8, PMC5386518; Xu, W et al. Med Sci Monit. 2019 May 20; 25:3762-3770). WDR5 is also connected to pancreatic cancer as an identified hit in an unbiased shRNA screen for patient derived cell growth and the modulator of Myc function (Carugo, A. et al. Cell Reports. 2016, 16, 133-147). Based on the increasing findings of WDR5's roles in tumor initiation and maintenance, the emerging importance of WDR5 in oncology is not unexpected. WDR5 has a canonical conformation that contains a central cavity, both H3 and MLL1 peptides interact with it via an arginine to the arginine binding site in the cavity (Schuetz, A. et al. EMBO J. 2006, 25, 4245-4252; Han, Z. et al. Mol Cell 2006, 22, 137-144; Couture, J. F. et al. Nat Sttuct Mol Biol 2006, 13, 698-703; Ruthenburg, A. J. et al. Nat Struct Mol Biol 2006, 13, 704-712). Thus, disruption of WDR5 from chromatin as the strategy to displace WDR5 binding partners such as Myc, which is deregulated in ˜50% of human malignancy from their target genes may provide a beneficial strategy to treat broaden population of patient. Taken these together, WDR5 arginine interacting pocket binders as the inhibitors for WDR5 or its cofactor function modulators could be beneficial for MLL-r leukemia and also a subset of solid tumors, either as single agent or in combination with other standard of cares.

### SUMMARY OF THE INVENTION

In one aspect, the invention provides a compound of the formula (I), or a pharmaceutically acceptable salt thereof:

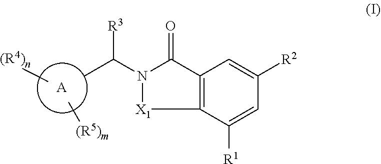

wherein:
X₁ is *—CH₂CH₂—**, *—CH═CH—**, *—N═CH—** or *—CH═N—**, where the * of X₁ indicates the point of attachment to the N and the ** of X₁ indicates the point of attachment to the phenyl ring;
A is selected from a 5-6 membered heteroaryl having 1 to 3 heteroatoms independently selected from N, O and S and a 9 to 14 membered heteroaryl having 1-4 heteroatoms independently selected from N, O and S;
R¹ is selected from H, halo, —R⁶, —CH₂R⁶, —CH₂NR^{1a}R^{1b}, C₁-C₆haloalkyl, a C₁-C₆alkyl substituted with 1 to 2 groups independently selected from —OR^{1a}, —S(═O)₂R¹² and —N(R¹²)₂, and a C₁-C₆haloalkyl substituted with 1 to 2 groups independently selected from C₁-C₆alkyl, —OR^{1a}, —S(═O)₂R¹² and —N(R¹²)₂;
   wherein,
      R^{1a} is selected from H, C₁-C₆alkyl, C₁-C₆haloalkyl, C₃-C₈cycloalkyl, and a 4-6 membered heterocycloalkyl having 1 to 2 heteroatoms independently selected from N and O, and
      R^{1b} is selected from H, C₁-C₆alkyl, C₁-C₆haloalkyl, C₃-C₈cycloalkyl, and a 4-6 membered heterocycloalkyl having 1 to 2 heteroatoms independently selected from N and O;
R² is selected from —(CH₂)R⁷, —(CH₂)₂R⁸ and —(CH₂)₂NR^{2a}R^{2b};
   wherein,
      R^{2a} is selected from H, C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy, and
      R^{2b} is selected from H, C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy;
R³ is selected from H and C₁-C₆alkyl;
each of R⁴ is independently selected from halo, C₁-C₆alkyl, cyano, C₁-C₆alkoxy, C₁-C₆haloalkoxy and —C(═O)N(R¹²)₂;
R⁵ selected from halo, C₁-C₆alkyl, cyano, C₁-C₆alkoxy, C₁-C₆haloalkoxy and —C(═O)N(R¹²)₂;
R⁶ is selected from phenyl, pyridinonyl, tetrahydropyridinyl, pyridazinonyl, a 4-6 membered heterocycloalkyl having 1 to 2 heteroatoms independently selected from N and O, a 5-6 membered heteroaryl having 1 to 3 heteroatoms independently selected from N, O and S, a 9 to 10 membered heteroaryl having 1-3 heteroatoms independently selected from N, O and S, C₃-C₈cycloalkyl,

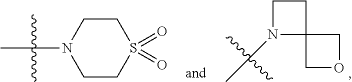 [SMILES: *N1CCS(=O)(=O)CC1 ; *N1CCC12COC2]

wherein the phenyl, pyridinonyl, pyridazinonyl, heterocycloalkyl and heteroaryl of R⁶ are optionally substituted with 1 to 3 R⁹ groups, and wherein each R⁹ is independently selected from C₁-C₆alkyl, C₁-C₆alkoxy, C₁-C₆haloalkyl, C₃-C₈cycloalkyl, halo, cyano, —N(R¹²)₂, —OH, —C(═O)R¹², —S(═O)₂R¹² and a C₁-C₆alkyl substituted with cyano, —N(R¹²)₂ or 1 to 2 —OH groups;
R⁷ is a 5-6 membered heteroaryl having 1 to 3 nitrogen atoms, optionally substituted with 1 to 3 R¹⁰ groups, and wherein each R¹⁰ is independently selected from C₁-C₆alkyl, C₁-C₆alkoxy, C₁-C₆haloalkyl, C₃-C₈cycloalkyl, halo, cyano, —N(R¹²)₂, —OH, and a C₁-C₆alkyl substituted with cyano, —N(R¹²)₂ or 1 to 2 —OH groups;
R⁸ is selected from a 4-6 membered heterocycloalkyl having 1 to 2 heteroatoms independently selected from N and O, optionally substituted with 1 to 2 R¹¹ groups, and wherein each R¹¹ is independently selected from C₁-C₆alkyl, C₁-C₆alkoxy, C₁-C₆haloalkyl, C₃-C₈cycloalkyl, halo, cyano, —N(R¹²)₂, —OH, and a C₁-C₆alkyl substituted with cyano, —N(R¹²)₂ or 1 to 2 —OH groups;
each R¹² is independently selected from H and C₁-C₆alkyl;
m is 0 or 1, and
n is 0, 1 or 2.

Compounds of formula (I), and subformulae thereof described herein, are inhibitors of WDR5, and are accordingly useful to treat a disease associated with excessive or undesired levels of activity of WDR5, in particular a cancer associated with excessive or undesired levels of activity of WDR5.

In another aspect, the invention provides pharmaceutical compositions comprising a compound of Formula (I), or subformulae thereof described herein, and one or more pharmaceutically acceptable carriers. These compositions are also useful to treat a disease associated with excessive or undesired levels of activity of WDR5, in particular a cancer associated with excessive or undesired levels of activity of WDR5. The compositions may also comprise one or more additional therapeutic agents, such as those described herein.

In another aspect, the invention provides a method to treat a disease characterized by excessive or undesired levels of activity of WDR5, wherein the method comprises administering to a subject in need of such treatment a therapeutically effective amount of a compound of Formula (I), or subformulae thereof described herein, or a pharmaceutical composition comprising a compound of Formula (I), or subformulae thereof described herein. In certain embodiments, the disease includes various forms of cancer, such as solid tumors, adenoma, bladder cancer, brain cancer, breast cancer, cervical cancer, colorectal cancer, colon cancer, epidermal carcinoma, follicular carcinoma, genitourinary cancers, glioblastoma, head and neck cancers, Hodgkin's disease, non-Hodgkin's lymphoma, hepatoma, kidney cancer, lung cancers such as small cell or non-small cell lung cancer, leukemias such as AML or CML, multiple myeloma, lymphoid disorders, skin cancers including melanoma, neuroblastoma, ovarian cancer, pancreatic cancer, prostate cancer, rectal cancer, sarcoma, testicular cancer, and thyroid cancer. In preferred embodiments the disease is melanoma, ovarian cancer, thyroid cancer, colon cancer, lung cancer, pancreatic cancer, cervical cancer, head and neck cancer, and leukemias including chronic myelomonocytic leukemia (CMML), AML and CML. In certain embodiments, the subject to be treated can be a mammal, and is preferably a human.

In another aspect, the invention provides a method to treat a disease characterized by excessive or undesired levels of activity of WDR5, wherein the method comprises administering to a subject in need of such treatment a compound of Formula (I), or subformulae thereof described herein, or a pharmaceutical composition comprising a compound of Formula (I), or subformulae thereof described herein. In certain embodiments, the disease includes various forms of cancer, such as solid tumors, adenoma, bladder cancer, brain cancer, breast cancer, cervical cancer, colorectal cancer, colon cancer, epidermal carcinoma, follicular carcinoma, genitourinary cancers, glioblastoma, head and neck cancers, Hodgkin's disease, non-Hodgkin's lymphoma, hepatoma, kidney cancer, lung cancers such as small cell or non-small cell lung cancer, leukemias such as AML or CML, multiple myeloma, lymphoid disorders, skin cancers including melanoma, neuroblastoma, ovarian cancer, pancreatic cancer, prostate cancer, rectal cancer, sarcoma, testicular cancer, and thyroid cancer. In preferred embodiments the disease is melanoma, ovarian cancer, thyroid cancer, colon cancer, lung cancer, pancreatic cancer, cervical cancer, head and neck cancer, and leukemias including chronic myelomonocytic leukemia (CMML), AML and CML. In certain embodiments, the subject to be treated can be a mammal, and is preferably a human.

In another aspect, the invention provides a compound of Formula (I), or subformulae thereof described herein, for use in the treatment of a disease characterized by excessive or undesired levels of activity of WDR5, wherein the disease includes various forms of cancer, such as solid tumors, adenoma, bladder cancer, brain cancer, breast cancer, cervical cancer, colorectal cancer, colon cancer, epidermal carcinoma, follicular carcinoma, genitourinary cancers, glioblastoma, head and neck cancers, Hodgkin's disease, non-Hodgkin's lymphoma, hepatoma, kidney cancer, lung cancers such as small cell or non-small cell lung cancer, leukemias such as AML or CML, multiple myeloma, lymphoid disorders, skin cancers including melanoma, neuroblastoma, ovarian cancer, pancreatic cancer, prostate cancer, rectal cancer, sarcoma, testicular cancer, and thyroid cancer. In preferred embodiments the disease is melanoma, ovarian cancer, thyroid cancer, colon cancer, lung cancer, pancreatic cancer, cervical cancer, head and neck cancer, and leukemias including chronic myelomonocytic leukemia (CMML), AML and CML.

In another aspect, the invention provides a pharmaceutical composition comprising a compound of Formula (I), or subformulae thereof described herein, for use in the treatment of a disease characterized by excessive or undesired levels of activity of WDR5, wherein the disease includes various forms of cancer, such as solid tumors, adenoma, bladder cancer, brain cancer, breast cancer, cervical cancer, colorectal cancer, colon cancer, epidermal carcinoma, follicular carcinoma, genitourinary cancers, glioblastoma, head and neck cancers, Hodgkin's disease, non-Hodgkin's lymphoma, hepatoma, kidney cancer, lung cancers such as small cell or non-small cell lung cancer, leukemias such as acute myelogenous leukemia (AML) or chronic myelogenous leukemia (CML), multiple myeloma, lymphoid disorders, skin cancers including melanoma, neuroblastoma, ovarian cancer, pancreatic cancer, prostate cancer, rectal cancer, sarcoma, testicular cancer, and thyroid cancer. In preferred embodiments the disease is melanoma, ovarian cancer, thyroid cancer, colon cancer, lung cancer, pancreatic cancer, cervical cancer, head and neck cancer, and leukemias including chronic myelomonocytic leukemia (CMML), AML and CML.

In another aspect, the invention provides the use of a compound of Formula (I), or subformulae thereof described herein, for the treatment of a disease characterized by excessive or undesired levels of activity of WDR5, wherein the disease includes various forms of cancer, such as solid tumors, adenoma, bladder cancer, brain cancer, breast cancer, cervical cancer, colorectal cancer, colon cancer, epidermal carcinoma, follicular carcinoma, genitourinary cancers, glioblastoma, head and neck cancers, Hodgkin's disease, non-Hodgkin's lymphoma, hepatoma, kidney cancer, lung cancers such as small cell or non-small cell lung cancer, leukemias such as acute myelogenous leukemia (AML) or chronic myelogenous leukemia (CML), multiple myeloma, lymphoid disorders, skin cancers including melanoma, neuroblastoma, ovarian cancer, pancreatic cancer, prostate cancer, rectal cancer, sarcoma, testicular cancer, and thyroid cancer. In preferred embodiments the disease is melanoma, ovarian cancer, thyroid cancer, colon cancer, lung cancer, pancreatic cancer, cervical cancer, head and neck cancer, and leukemias including chronic myelomonocytic leukemia (CMML), AML and CML.

In another aspect, the invention provides the use of a pharmaceutical composition comprising a compound of Formula (I), or subformulae thereof described herein, for the treatment of a disease characterized by excessive or undesired levels of activity of WDR5, wherein the disease includes various forms of cancer, such as solid tumors, adenoma, bladder cancer, brain cancer, breast cancer, cervical cancer, colorectal cancer, colon cancer, epidermal carcinoma, follicular carcinoma, genitourinary cancers, glioblastoma, head and neck cancers, Hodgkin's disease, non-Hodgkin's lymphoma, hepatoma, kidney cancer, lung cancers such as small cell or non-small cell lung cancer, leukemias such as acute myelogenous leukemia (AML) or chronic myelogenous leukemia (CML), multiple myeloma, lymphoid disorders, skin cancers including melanoma, neuroblastoma, ovarian cancer, pancreatic cancer, prostate cancer, rectal cancer, sarcoma, testicular cancer, and thyroid cancer. In preferred embodiments the disease is melanoma, ovarian cancer, thyroid cancer, colon cancer, lung cancer, pancreatic cancer, cervical cancer, head and neck cancer, and leukemias including chronic myelomonocytic leukemia (CMML), AML and CML.

In another aspect, the invention provides the use of a compound of Formula (I), or subformulae thereof described herein, in the manufacture of a medicament for the treatment of a disease characterized by excessive or undesired levels of activity of WDR5, wherein the disease includes various forms of cancer, such as solid tumors, adenoma, bladder cancer, brain cancer, breast cancer, cervical cancer, colorectal cancer, colon cancer, epidermal carcinoma, follicular carcinoma, genitourinary cancers, glioblastoma, head and neck cancers, Hodgkin's disease, non-Hodgkin's lymphoma, hepatoma, kidney cancer, lung cancers such as small cell or non-small cell lung cancer, leukemias such as acute myelogenous leukemia (AML) or chronic myelogenous leukemia (CML), multiple myeloma, lymphoid disorders, skin cancers including melanoma, neuroblastoma, ovarian cancer, pancreatic cancer, prostate cancer, rectal cancer, sarcoma, testicular cancer, and thyroid cancer. In preferred embodiments the disease is melanoma, ovarian cancer, thyroid cancer, colon cancer, lung cancer, pancreatic cancer, cervical cancer, head and neck cancer, and leukemias including chronic myelomonocytic leukemia (CMML), AML and CML.

In another aspect, the invention provides methods of making the compounds of Formula (I) as well as key intermediate compounds useful for making the compounds of the invention.

### DETAILED DESCRIPTION

Various enumerated embodiments of the present invention are described herein. It will be recognized that features specified in each embodiment may be combined with other specified features to provide further embodiments of the present invention.

### Definitions

The following definitions apply unless otherwise provided or apparent from context:

The term “alkyl,” as used herein, refers to a fully saturated branched or straight chain hydrocarbon. In certain embodiments an alkyl group is a “C₁-C₂alkyl”, “C₁-C₃alkyl”, “C₁-C₄alkyl”, “C₁-C₅alkyl”, “C₁-C₆alkyl”, “C₁-C₇alkyl”, “C₁-C₈alkyl”, “C₁-C₉alkyl” or “C₁-C₁₀alkyl”, wherein the terms “C₁-C₂alkyl”, “C₁-C₃alkyl”, “C₁-C₄alkyl”, “C₁-C₅alkyl”, “C₁-C₆alkyl”, “C₁-C₇alkyl”, “C₁-C₈alkyl”, “C₁-C₉alkyl” and “C₁-C₁₀alkyl”, as used herein, refer to an alkyl group containing at least 1, and at most 2, 3, 4, 5, 6, 7, 8, 9 or 10 carbon atoms, respectively. Non-limiting examples of alkyl groups include methyl, ethyl, n-propyl, isopropyl, n-butyl, isobutyl, sec-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl.

The term “alkoxy”, as used herein, refers to —O-alkyl or -alkyl-O—, wherein the “alkyl” group is as as defined herein. In certain embodiments an alkoxy group is a “C₁-C₂alkoxy”, “C₁-C₃alkoxy”, “C₁-C₄alkoxy”, “C₁-C₅alkoxy”, “C₁-C₆alkoxy”, “C₁-C₇alkoxy”, “C₁-C₈alkoxy”, “C₁-C₉alkoxy” or “C₁-C₁₀alkoxy”, wherein the terms “C₁-C₃alkoxy”, “C₁-C₄alkoxy”, “C₁-C₅alkoxy”, “C₁-C₆alkoxy”, “C₁-C₇alkoxy”, “C₁-C₈alkoxy”, “C₁-C₉alkoxy” and “C₁-C₁₀alkoxy”, as used herein refer to —O—C₁-C₂alkyl, —O—C₁-C₃alkyl, —O—C₁-C₄alkyl, —O—C₁-C₅alkyl, —O—C₁-C₆alkyl, —O—C₁-C₇alkyl, —O—C₁-C₈alkyl, —O—C₁-C₉alkyl or —O—C₁-C₁₀alkyl, respectively. Non-limiting examples of “alkoxy” groups include methoxy, ethoxy, n-propoxy, isopropoxy, n-butoxy, isobutoxy, sec-butoxy, tert-butoxy, n-pentoxy, isopentoxy, hexoxy, heptoxy, octoxy, nonoxy, decoxy and the like.

The term “C₃-C₈cycloalkyl” as used herein, refers to a saturated, monocyclic hydrocarbon ring system having 3 to 8 carbon atoms as ring members. Non-limiting examples of such “C₃-C₈cycloalkyl” groups include cyclopropyl, cyclobutyl, cyclopentyl, cyclohexyl, cycloheptyl and cyclooctyl groups.

The term “haloalkyl” as used herein, refers to an alkyl as defined herein, wherein at least one of the hydrogen atoms of the alkyl is replaced by a halo group as defined herein. The haloalkyl can be monohaloalkyl, dihaloalkyl, trihaloalkyl, or polyhaloalkyl including perhaloalkyl. A monohaloalkyl can have one iodo, bromo, chloro or fluoro within the alkyl group. Dihaloalkyl and polyhaloalkyl groups can have two or more of the same halo atoms or a combination of different halo groups within the alkyl. Typically the polyhaloalkyl contains up to 6, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-alkyl refers to an alkyl having all hydrogen atoms replaced with halo atoms, e.g., trifluoromethyl. Preferred haloalkyl groups, unless specified otherwise, include monofluoro-, difluoro- and trifluoro-substituted methyl and ethyl groups, e.g. CF₃, CHF₂, CH₂F, CH₂CHF₂ and CH₂CF₃.

The term “C₁-C₆haloalkyl” as used herein, refers to the respective “C₁-C₆alkyl”, as defined herein, wherein at least one of the hydrogen atoms of the “C₁-C₆alkyl” is replaced by a halo atom. The C₁-C₆haloalkyl groups can be monoC₁-C₆haloalkyl, wherein such C₁-C₆haloalkyl groups have one iodo, one bromo, one chloro or one fluoro. Additionally, the C₁-C₆haloalkyl groups can be diC₁-C₆haloalkyl wherein such C₁-C₆haloalkyl groups can have two halo atoms independently selected from iodo, bromo, chloro or fluoro. Furthermore, the C₁-C₆haloalkyl groups can be polyC₁-C₆haloalkyl wherein such C₁-C₆haloalkyl groups can have two or more of the same halo atoms or a combination of two or more different halo atoms. Such polyC₁-C₆haloalkyl can be perhaloC₁-C₆haloalkyl where all the hydrogen atoms of the respective C₁-C₆alkyl have been replaced with halo atoms and the halo atoms can be the same or a combination of different halo atoms. Non-limiting examples of “C₁-C₆haloalkyl” groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, fluoroethyl, difluoroethyl, trifluoroethyl, difluoropropyl, dichloroethyl and dichloropropyl.

The term “haloalkoxy” as used herein, refers to the group —Ohaloalkyl wherein at least one of the hydrogen atoms of the alkyl group of the alkoxy is replaced by a halo group as defined herein. The haloalkoxy can be monohaloalkoxy, dihaloalkoxy, trihaloalkoxy, or polyhaloalkoxy including perhaloalkoxy. A monohaloalkoxy can have one iodo, bromo, chloro or fluoro within the alkyl group. Dihaloalkoxy and polyhaloalkoxy groups can have two or more of the same halo atoms or a combination of different halo groups within the alkyl. Typically the polyhaloalkoxy contains up to 6, or 4, or 3, or 2 halo groups. Non-limiting examples of haloalkoxy include fluoromethoxy, difluoromethoxy, trifluoromethoxy, chloromethoxy, dichloromethoxy, trichloromethoxy, pentafluoroethoxy, heptafluoropropoxy, difluorochloromethoxy, dichlorofluoromethoxy, difluoroethoxy, difluoropropoxy, dichloroethoxy and dichloropropoxy. A perhalo-alkoxy refers to an alkoxy having all hydrogen atoms replaced with halo atoms, e.g., trifluoromethoxy. Preferred haloalkoxy groups, unless specified otherwise, include monofluoro-, difluoro- and trifluoro-substituted methoxy and ethoxygroups, e.g. —OCF₃, —OCHF₂, —OCH₂F, —OCH₂CHF₂ and —OCH₂CF₃.

The term “C₁-C₆haloalkoxy” as used herein, refers to the group —OC₁-C₆haloalkyl, wherein at least one of the hydrogen atoms of the “C₁-C₆alkyl” of the “C₁-C₆alkoxy” is replaced by a halo atom, i.e. The C₁-C₆haloalkoxy groups can be monoC₁-C₆haloalkoxy, wherein such C₁-C₆haloalkoxy groups have one iodo, one bromo, one chloro or one fluoro. Additionally, the C₁-C₆haloalkoxy groups can be diC₁-C₆haloalkoxy wherein such C₁-C₆haloalkoxy groups can have two halo atoms independently selected from iodo, bromo, chloro or fluoro. Furthermore, the C₁-C₆haloalkoxy groups can be polyC₁-C₆haloalkoxy wherein such C₁-C₆haloalkoxy groups can have two or more of the same halo atoms or a combination of two or more different halo atoms. Such polyC₁-C₆haloalkoxy can be perhaloC₁-C₆haloalkoxy where all the hydrogen atoms of the respective C₁-C₆alkoxy have been replaced with halo atoms and the halo atoms can be the same or a combination of different halo atoms. Non-limiting examples of “C₁-C₆haloalkoxy” groups include fluoromethoxy, difluoromethoxy, trifluoromethoxy, chloromethoxy, dichloromethoxy, trichloromethoxy, pentafluoroethoxy, heptafluoropropoxy, difluorochloromethoxy, dichlorofluoromethoxy, fluoroethoxy, difluoroethoxy, trifluoroethoxy, difluoropropoxy, dichloroethoxy and dichloropropoxy.

The terms “halo” or “halogen” as used herein, refer to fluoro, chloro, bromo and iodo.

The term “heteroaryl,” as used herein, refers to i) an aromatic, 5-6 membered monocyclic ring system having 1 to 3 heteroatoms independently selected from the heteroatoms N, O and S, ii) an aromatic, 5-6 membered monocyclic ring system having 1 to 3 nitrogen atoms, and iii) an aromatic, 9-10 membered fused bicyclic ring system having 1 to 3 heteroatoms independently selected from the heteroatoms N, O and S. Non-limiting examples of heteroaryl groups, as used herein, include benzofuranyl, benzo[c]thiophenyl, benzothiophenyl, benzoxazolyl, benzthiazolyl, benzimidazolyl, cinnolinyl, furazanyl, furyl, imidazolyl, indolyl, indolizinyl, indazolyl, isoindolyl, isoquinolinyl, isoxazolyl, isothiazolyl, naphthalenyl, oxazolyl, oxaindolyl, oxadiazolyl, pyrazolyl, pyrrolyl, phthalazinyl, pyridyl, pyridazinyl, pyrazinyl, pyrimidinyl, quinoxalinyl, quinolinyl, quinazolinyl, thiazolyl, thiadiazolyl, thienyl, triazinyl, and triazolyl. In preferred embodiments a heteroaryl group is a pyridyl.

The term “heteroatoms” as used herein, refers to nitrogen (N), oxygen (O) or sulfur (S) atoms.

The term “heterocycloalkyl,” as used herein refers to i) a monocyclic ring structure having 4 to 6 ring members, wherein one to two of the ring members are independently selected from N, NH, NR³⁶, O or —S—, wherein R³⁶ is C₁-C₆alkyl and ii) a fused bicyclic ring structure having 8 to 10 ring members, wherein one to two of the ring members are independently selected from N, NH, NR³⁶, O or —S—, wherein R³⁶ is C₁-C₆alkyl. Non-limiting examples of 4-6 membered heterocycloalkyl groups, as used herein, include azetadinyl, azetadin-1-yl, azetadin-2-yl, azetadin-3-yl, oxetanyl, oxetan-2-yl, oxetan-3-yl, oxetan-4-yl, thietanyl, thietan-2-yl, thietan-3-yl, thietan-4-yl, pyrrolidinyl, pyrrolidin-1-yl, pyrrolidin-2-yl, pyrrolidin-3-yl, pyrrolidin-4-yl, pyrrolidin-5-yl, tetrahydrofuranyl, tetrahydrofuran-2-yl, tetrahydrofuran-3-yl, tetrahydrofuran-4-yl, tetrahydrofuran-5-yl, tetrahydrothienyl, tetrahydrothien-2-yl, tetrahydrothien-3-yl, tetrahydrothien-4-yl, tetrahydrothien-5-yl, piperidinyl, piperidin-1-yl, piperidin-2-yl, piperidin-3-yl, piperidin-4-yl, piperidin-5-yl, piperidin-6-yl, tetrahydropyranyl, tetrahydropyran-2-yl, tetrahydropyran-3-yl, tetrahydropyran-4-yl, tetrahydropyran-5-yl, tetrahydropyran-6-yl, tetrahydrothiopyranyl, tetrahydrothiopyran-2-yl, tetrahydrothiopyran-3-yl, tetrahydrothiopyran-4-yl, tetrahydrothiopyran-5-yl, tetrahydrothiopyran-6-yl, piperazinyl, piperazin-1-yl, piperazin-2-yl, piperazin-3-yl, piperazin-4-yl, piperazin-5-yl, piperazin-6-yl, morpholinyl, morpholin-2-yl, morpholin-3-yl, morpholin-4-yl, morpholin-5-yl, morpholin-6-yl, thiomorpholinyl, thiomorpholin-2-yl, thiomorpholin-3-yl, thiomorpholin-4-yl, thiomorpholin-5-yl, thiomorpholin-6-yl, oxathianyl, oxathian-2-yl, oxathian-3-yl, oxathian-5-yl, oxathian-6-yl, dithianyl, dithian-2-yl, dithian-3-yl, dithian-5-yl, dithian-6-yl, dioxolanyl, dioxolan-2-yl, dioxolan-4-yl, dioxolan-5-yl, thioxanyl, thioxan-2-yl, thioxan-3-yl, thioxan-4-yl, thioxan-5-yl, dithiolanyl, dithiolan-2-yl, dithiolan-4-yl, dithiolan-5-yl, pyrazolidinyl, pyrazolidin-1-yl, pyrazolidin-2-yl, pyrazolidin-3-yl, pyrazolidin-4-yl, pyrazolidin-5-yl, 2-azabicyclo[4.2.0]octanyl, octahydro-1H-cyclopenta[b]pyridine and decahydroquinoline.

As used herein, the term “hetero atoms” refers to nitrogen (N), oxygen (O) or sulfur (S) atoms, in particular nitrogen or oxygen, unless otherwise provided.

The terms “combination” or “pharmaceutical combination,” as used herein, refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent.

The term “combination therapy” or “in combination with” or “pharmaceutical combination” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent being administered prior to, concurrent with, or sequentially to each other with no specific time limits. In each case, the treatment regimen will provide beneficial effects of the drug combination in treating the conditions or disorders described herein.

As used herein the term “co-administer” refers to the presence of two active agents in the blood of an individual. Active agents that are co-administered can be concurrently or sequentially delivered.

The terms “composition” or “pharmaceutical composition,” as used herein, refers to a compound of the present invention, or a pharmaceutically acceptable salt thereof, together with at least one pharmaceutically acceptable carrier, in a form suitable for oral or parenteral administration.

A “patient,” “subject” or “individual” are used interchangeably and refer to either a human or non-human animal. The term includes mammals such as humans. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

The term “an optical isomer” or “a stereoisomer”, as used herein, refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)— or (S)—.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22^{nd} Ed. Pharmaceutical Press, 2013, pp. 1049-1070).

The phrase “pharmaceutically acceptable” indicates that the substance, composition or dosage form must be compatible chemically and/or toxicologically, with the other ingredients comprising a formulation, and/or the mammal being treated therewith.

The term “a subject in need of such treatment”, refers to a subject which would benefit biologically, medically or in quality of life from such treatment.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by a kinase such as WDR5 or (ii) associated with activity of a kinase such as WDR5, or (iii) characterized by activity (normal or abnormal) of WDR5; or (2) reduce or inhibit the activity of WDR5 or (3) reduce or inhibit the expression of WDR5.

In another non-limiting embodiment, the term “a therapeutically effective amount” refers to the amount of the compound of the present invention that, when administered to a cell, or a tissue, or a non-cellular biological material, or a medium, is effective to at least partially reduce or inhibit the activity of WDR5, or at least partially reduce or inhibit the expression of WDR5.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, a subject is “in need of” a treatment if such subject would benefit biologically, medically or in quality of life from such treatment.

As used herein, the term “prevent”, “preventing” or “prevention” of any disease or disorder refers to the prophylactic treatment of the disease or disorder; or delaying the onset or progression of the disease or disorder.

The compound names provided herein were obtained using ChemDraw Ultra version 14.0 (CambridgeSoft®).

As used herein, the term “a,” “an,” “the” and similar terms used in the context of the present invention (especially in the context of the claims) are to be construed to cover both the singular and plural unless otherwise indicated herein or clearly contradicted by the context.

### Compounds of the Invention

The invention therefore provides a compound having the structure of formula (I), or pharmaceutically acceptable salt thereof:

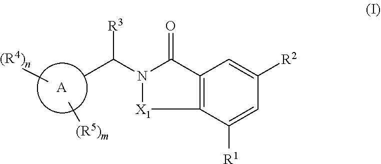

wherein:
X₁ is *—CH₂CH₂—**, *—CH═CH—**, *—N═CH—** or *—CH═N—**, where the * of X₁ indicates the point of attachment to the N and the ** of X₁ indicates the point of attachment to the phenyl ring;
A is selected from a 5-6 membered heteroaryl having 1 to 3 heteroatoms independently selected from N, O and S and a 9 to 14 membered heteroaryl having 1-4 heteroatoms independently selected from N, O and S;
R¹ is selected from H, halo, —R⁶, —CH₂R⁶, —CH₂NR^{1a}R^{1b}, C₁-C₆haloalkyl, a C₁-C₆alkyl substituted with 1 to 2 groups independently selected from —OR^{1a}, —S(═O)₂R¹² and —N(R¹²)₂, and a C₁-C₆haloalkyl substituted with 1 to 2 groups independently selected from C₁-C₆alkyl, —OR^{1a}, —S(═O)₂R¹² and —N(R¹²)₂;
   wherein,
      R^{1a} is selected from H, C₁-C₆alkyl, C₁-C₆haloalkyl, C₃-C₈cycloalkyl, and a 4-6 membered heterocycloalkyl having 1 to 2 heteroatoms independently selected from N and O, and
      R^{1b} is selected from H, C₁-C₆alkyl, C₁-C₆haloalkyl, C₃-C₈cycloalkyl, and a 4-6 membered heterocycloalkyl having 1 to 2 heteroatoms independently selected from N and O;
R² is selected from —(CH₂)R⁷, —(CH₂)₂R⁸ and —(CH₂)₂NR^{2a}R^{2b};
   wherein,
      R^{2a} is selected from H, C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy, and
      R^{2b} is selected from H, C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy;
R³ is selected from H and C₁-C₆alkyl;
each of R⁴ is independently selected from halo, C₁-C₆alkyl, cyano, C₁-C₆alkoxy, C₁-C₆haloalkoxy and —C(═O)N(R¹²)₂;
R⁵ selected from halo, C₁-C₆alkyl, cyano, C₁-C₆alkoxy, C₁-C₆haloalkoxy and —C(═O)N(R¹²)₂;
R⁶ is selected from phenyl, pyridinonyl, tetrahydropyridinyl, pyridazinonyl, a 4-6 membered heterocycloalkyl having 1 to 2 heteroatoms independently selected from N and O, a 5-6 membered heteroaryl having 1 to 3 heteroatoms independently selected from N, O and S, a 9 to 10 membered heteroaryl having 1-3 heteroatoms independently selected from N, O and S, C₃-C₈cycloalkyl,

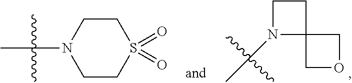 [SMILES: *N1CCS(=O)(=O)CC1 ; *N1CCC12COC2]

wherein the phenyl, pyridinonyl, pyridazinonyl, heterocycloalkyl and heteroaryl of R⁶ are optionally substituted with 1 to 3 R⁹ groups, and wherein each R⁹ is independently selected from C₁-C₆alkyl, C₁-C₆alkoxy, C₁-C₆haloalkyl, C₃-C₈cycloalkyl, halo, cyano, —N(R¹²)₂, —OH, —C(═O)R¹², —S(═O)₂R¹² and a C₁-C₆alkyl substituted with cyano, —N(R¹²)₂ or 1 to 2 —OH groups;
R⁷ is a 5-6 membered heteroaryl having 1 to 3 nitrogen atoms, optionally substituted with 1 to 3 R¹⁰ groups, and wherein each R¹⁰ is independently selected from C₁-C₆alkyl, C₁-C₆alkoxy, C₁-C₆haloalkyl, C₃-C₈cycloalkyl, halo, cyano, —N(R¹²)₂, —OH, and a C₁-C₆alkyl substituted with cyano, —N(R¹²)₂ or 1 to 2 —OH groups;
R⁸ is selected from a 4-6 membered heterocycloalkyl having 1 to 2 heteroatoms independently selected from N and O, optionally substituted with 1 to 2 R¹¹ groups, and wherein each R¹¹ is independently selected from C₁-C₆alkyl, C₁-C₆alkoxy, C₁-C₆haloalkyl, C₃-C₈cycloalkyl, halo, cyano, —N(R¹²)₂, —OH, and a C₁-C₆alkyl substituted with cyano, —N(R¹²)₂ or 1 to 2 —OH groups;
each R¹² is independently selected from H and C₁-C₆alkyl;
m is 0 or 1, and
n is 0, 1 or 2.

Unless specified otherwise, the term “compound of the invention”, “compounds of the invention”, “compound of the present invention” or “compounds of the present invention” refers to a compound or compounds of formula (I), subformulae thereof (such as formula (Ia), formula (Ib), formula (Ic), formula (Id), formula (Ie), formula (If) and formula (Ig) and exemplified compounds, and salts thereof, as well as all stereoisomers (including diastereoisomers and enantiomers), rotamers, tautomers and isotopically labeled compounds (including deuterium substitutions), as well as inherently formed moieties (e.g., polymorphs, solvates and/or hydrates).

Certain aspects and examples of the compounds of the present invention are provided in the following listing of additional, enumerated embodiments. It will be recognized that features specified in each embodiment may be combined with other specified features to provide further embodiments of the present invention.

Embodiment 1. The compound of formula (I), or pharmaceutically acceptable salt thereof, wherein:
X₁ is *—CH₂CH₂—**, *—CH═CH—**, *—N═CH—** or *—CH═N—**, where the * of X₁ indicates the point of attachment to the N and the ** of X₁ indicates the point of attachment to the phenyl ring;
A is selected from

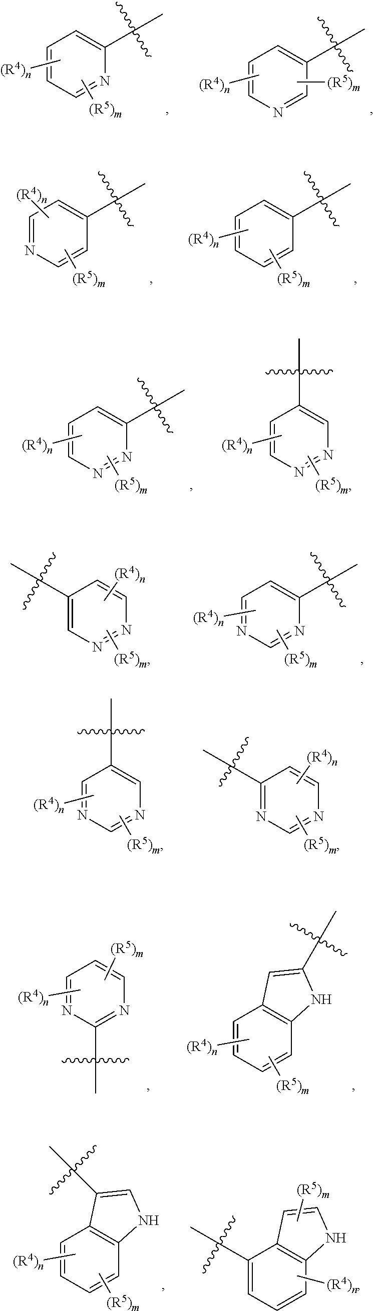

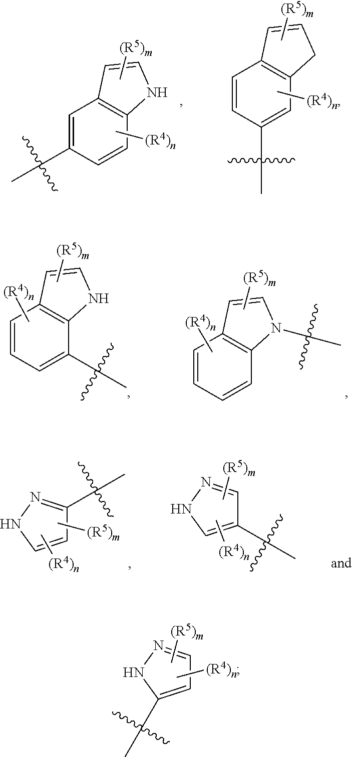

R¹ is selected from H, halo, —R⁶, —CH₂R⁶, —CH₂NR^{1a}R^{1b}, C₁-C₆haloalkyl, a C₁-C₆alkyl substituted with 1 to 2 groups independently selected from —OR^{1a}, —S(═O)₂R¹² and —N(R¹²)₂, and a C₁-C₆haloalkyl substituted with 1 to 2 groups independently selected from C₁-C₆alkyl, —OR^{1a}, —S(═O)₂R¹² and —N(R¹²)₂;
   wherein,
      R^{1a} is selected from H, C₁-C₆alkyl, C₁-C₆haloalkyl, C₃-C₈cycloalkyl, and a 4-6 membered heterocycloalkyl having 1 to 2 heteroatoms independently selected from N and O, and
      R^{1b} is selected from H, C₁-C₆alkyl, C₁-C₆haloalkyl, C₃-C₈cycloalkyl, and a 4-6 membered heterocycloalkyl having 1 to 2 heteroatoms independently selected from N and O;
R² is selected from —(CH₂)R⁷, —(CH₂)₂R⁸ and —(CH₂)₂NR^{2a}R^{2b};
   wherein,
      R^{2a} is selected from H, C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy, and
      R^{2b} is selected from H, C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy;
R³ is selected from H and C₁-C₆alkyl;
each of R⁴ is independently selected from halo, C₁-C₆alkyl, cyano, C₁-C₆alkoxy, C₁-C₆haloalkoxy and —C(═O)N(R¹²)₂;
R⁵ selected from halo, C₁-C₆alkyl, cyano, C₁-C₆alkoxy, C₁-C₆haloalkoxy and —C(═O)N(R¹²)₂;
R⁶ is selected from phenyl, pyridinonyl, tetrahydropyridinyl, pyridazinonyl, a 4-6 membered heterocycloalkyl having 1 to 2 heteroatoms independently selected from N and O, a 5-6 membered heteroaryl having 1 to 3 heteroatoms independently selected from N, O and S, a 9 to 10 membered heteroaryl having 1-3 heteroatoms independently selected from N, O and S, C₃-C₈cycloalkyl,

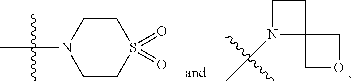 [SMILES: *N1CCS(=O)(=O)CC1 ; *N1CCC12COC2]

wherein the phenyl, pyridinonyl, pyridazinonyl, heterocycloalkyl and heteroaryl of R⁶ are optionally substituted with 1 to 3 R⁹ groups, and wherein each R⁹ is independently selected from C₁-C₆alkyl, C₁-C₆alkoxy, C₁-C₆haloalkyl, C₃-C₈cycloalkyl, halo, cyano, —N(R¹²)₂, —OH, —C(═O)R¹², —S(═O)₂R¹² and a C₁-C₆alkyl substituted with cyano, —N(R¹²)₂ or 1 to 2 —OH groups;
R⁷ is a 5-6 membered heteroaryl having 1 to 3 nitrogen atoms, optionally substituted with 1 to 3 R¹⁰ groups, and wherein each R¹⁰ is independently selected from C₁-C₆alkyl, C₁-C₆alkoxy, C₁-C₆haloalkyl, C₃-C₈cycloalkyl, halo, cyano, —N(R¹²)₂, —OH, and a C₁-C₆alkyl substituted with cyano, —N(R¹²)₂ or 1 to 2 —OH groups;
R⁸ is a 4-6 membered heterocycloalkyl having 1 to 2 heteroatoms independently selected from N and O, optionally substituted with 1 to 2 R¹¹ groups, and wherein each R¹¹ is independently selected from C₁-C₆alkyl, C₁-C₆alkoxy, C₁-C₆haloalkyl, C₃-C₈cycloalkyl, halo, cyano, —N(R¹²)₂, —OH, and a C₁-C₆alkyl substituted with cyano, —N(R¹²)₂ or 1 to 2 —OH groups;
each R¹² is independently selected from H and C₁-C₆alkyl;
m is 0 or 1, and
n is 0, 1 or 2.
   Embodiment 2. The compound of formula (I), or pharmaceutically acceptable salt thereof, wherein:
X₁ is *—CH₂CH₂—**, *—CH═CH—**, *—N═CH—** or *—CH═N—**, where the * of X₁ indicates the point of attachment to the N and the ** of X₁ indicates the point of attachment to the phenyl ring;
A is selected from

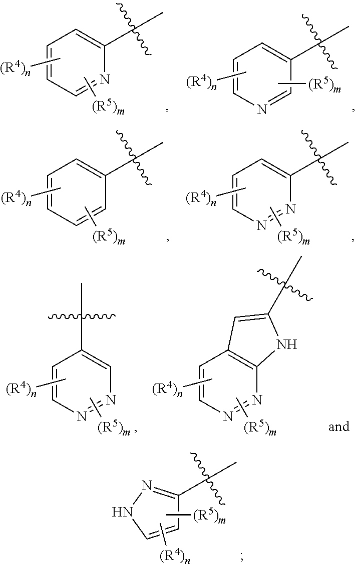

R¹ is selected from H, halo, —R⁶, —CH₂R⁶, —CH₂NR^{1a}R^{1b}, C₁-C₆haloalkyl, a C₁-C₆alkyl substituted with 1 to 2 groups independently selected from —OR^{1a}, —S(═O)₂R¹² and —N(R¹²)₂, and a C₁-C₆haloalkyl substituted with 1 to 2 groups independently selected from C₁-C₆alkyl, —OR^{1a}, —S(═O)₂R¹² and —N(R¹²)₂;
   wherein,
      R^{1a} is selected from H, C₁-C₆alkyl, C₁-C₆haloalkyl, C₃-C₈cycloalkyl, and a 4-6 membered heterocycloalkyl having 1 to 2 heteroatoms independently selected from N and O, and
      R^{1b} is selected from H, C₁-C₆alkyl, C₁-C₆haloalkyl, C₃-C₈cycloalkyl, and a 4-6 membered heterocycloalkyl having 1 to 2 heteroatoms independently selected from N and O;
R² is selected from —(CH₂)R⁷, —(CH₂)₂R⁸ and —(CH₂)₂NR^{2a}R^{2b};
   wherein,
      R^{2a} is selected from H, C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy, and
      R^{2b} is selected from H, C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy;
R³ is selected from H and C₁-C₆alkyl;
each of R⁴ is independently selected from halo, C₁-C₆alkyl, cyano, C₁-C₆alkoxy, C₁-C₆haloalkoxy and —C(═O)N(R¹²)₂;
R⁵ selected from halo, C₁-C₆alkyl, cyano, C₁-C₆alkoxy, C₁-C₆haloalkoxy and —C(═O)N(R¹²)₂;
R⁶ is selected from phenyl, pyridinonyl, tetrahydropyridinyl, pyridazinonyl, a 4-6 membered heterocycloalkyl having 1 to 2 heteroatoms independently selected from N and O, a 5-6 membered heteroaryl having 1 to 3 heteroatoms independently selected from N, O and S, a 9 to 10 membered heteroaryl having 1-3 heteroatoms independently selected from N, O and S, C₃-C₈cycloalkyl,

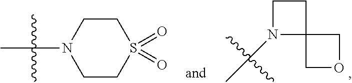 [SMILES: *N1CCS(=O)(=O)CC1 ; *N1CCC12COC2]

wherein the phenyl, pyridinonyl, pyridazinonyl, heterocycloalkyl and heteroaryl of R⁶ are optionally substituted with 1 to 3 R⁹ groups, and wherein each R⁹ is independently selected from C₁-C₆alkyl, C₁-C₆alkoxy, C₁-C₆haloalkyl, C₃-C₈cycloalkyl, halo, cyano, —N(R¹²)₂, —OH, —C(═O)R¹², —S(═O)₂R¹² and a C₁-C₆alkyl substituted with cyano, —N(R¹²)₂ or 1 to 2 —OH groups;
R⁷ is a 5-6 membered heteroaryl having 1 to 3 nitrogen atoms, optionally substituted with 1 to 3 R¹⁰ groups, and wherein each R¹⁰ is independently selected from C₁-C₆alkyl, C₁-C₆alkoxy, C₁-C₆haloalkyl, C₃-C₈cycloalkyl, halo, cyano, —N(R¹²)₂, —OH, and a C₁-C₆alkyl substituted with cyano, —N(R¹²)₂ or 1 to 2 —OH groups;
R⁸ is a 4-6 membered heterocycloalkyl having 1 to 2 heteroatoms independently selected from N and O, optionally substituted with 1 to 2 R¹¹ groups, and wherein each R¹¹ is independently selected from C₁-C₆alkyl, C₁-C₆alkoxy, C₁-C₆haloalkyl, C₃-C₈cycloalkyl, halo, cyano, —N(R¹²)₂, —OH, and a C₁-C₆alkyl substituted with cyano, —N(R¹²)₂ or 1 to 2 —OH groups;
each R¹² is independently selected from H and C₁-C₆alkyl;
m is 0 or 1, and
n is 0, 1 or 2.
   Embodiment 3. The compound of formula (I) or Embodiments 1 to 2, wherein X₁ is *—CH₂CH₂—**, and the compound has the structure of formula (Ia), or pharmaceutically acceptable salt thereof,

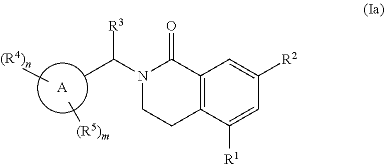

where R¹, R², R³, R⁴, R⁵, n and m are as defined for Formula (I) above.
Embodiment 4. The compound of formula (I) or Embodiments 1 to 2, wherein X₁ is *—CH═CH—**, and the compound has the structure of formula (Ib), or pharmaceutically acceptable salt thereof,

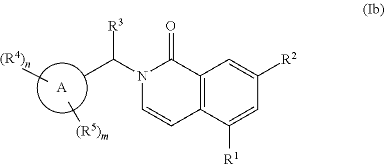

where R¹, R², R³, R⁴, R⁵, n and m are as defined for Formula (I) above.
Embodiment 5. The compound of formula (I) or Embodiments 1 to 2, wherein X₁ is *—N═CH—**, and the compound has the structure of formula (Ic), or pharmaceutically acceptable salt thereof,

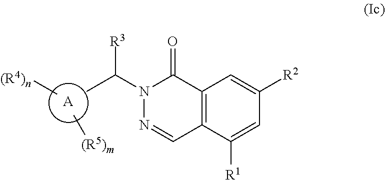

where R¹, R², R³, R⁴, R⁵, n and m are as defined for Formula (I) above.
Embodiment 6. The compound of formula (I) or Embodiments 1 to 2, wherein X₁ is *—CH═N—**, and the compound has the structure of formula (Id), or pharmaceutically acceptable salt thereof,

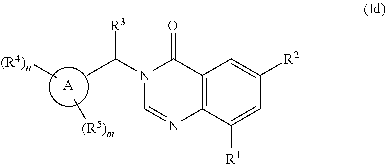

where R¹, R², R³, R⁴, R⁵, n and m are as defined for Formula (I) above.
Embodiment 7. The compound of formula (I) or any one of Embodiments 1 to 6, or pharmaceutically acceptable salt thereof, wherein A is

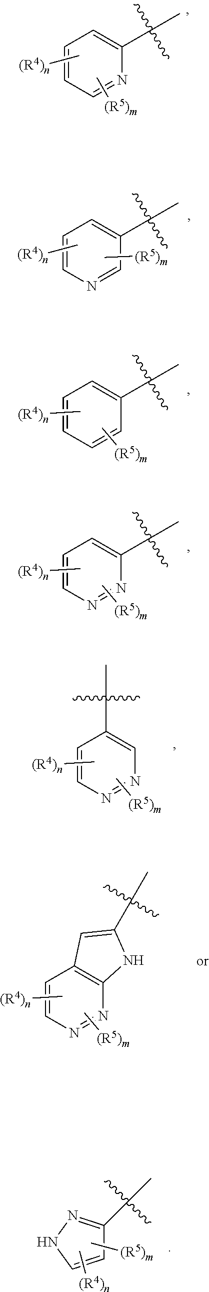

Embodiment 8. The compound of formula (I) or any one of Embodiments 1 to 6, or pharmaceutically acceptable salt thereof, wherein

### A is

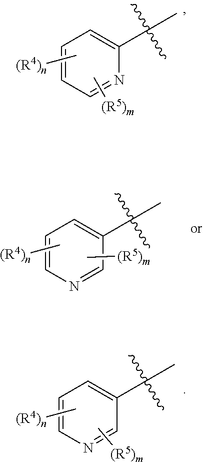

Embodiment 9. The compound of formula (I) or any one of Embodiments 1 to 6, or pharmaceutically acceptable salt thereof, wherein

### A is

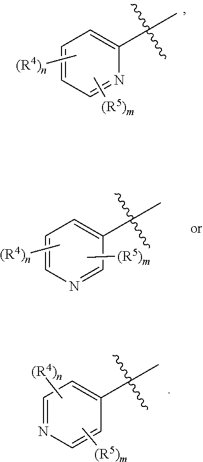

Embodiment 10. The compound of formula (I) or any one of Embodiments 1 to 6, or pharmaceutically acceptable salt thereof, wherein

### A is

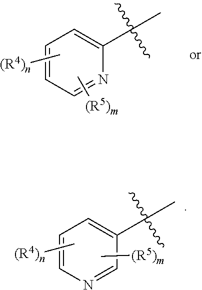

Embodiment 11. The compound of formula (I) or any one of Embodiments 1 to 6, or pharmaceutically acceptable salt thereof, wherein

### A is

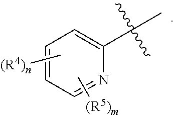

Embodiment 12. The compound of formula (I) or any one of Embodiments 1 to 6, or pharmaceutically acceptable salt thereof, wherein

### A is

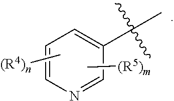

Embodiment 13. The compound of formula (I) or any one of Embodiments 1 to 6, or pharmaceutically acceptable salt thereof, wherein

### A is

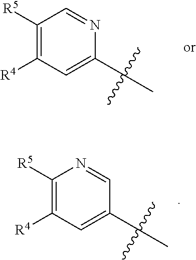

Embodiment 14. The compound of formula (I) or any one of Embodiments 1 to 6, or pharmaceutically acceptable salt thereof, wherein

### A is

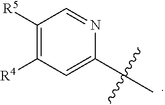

Embodiment 15. The compound of formula (I) or any one of Embodiments 1 to 6, or pharmaceutically acceptable salt thereof, wherein

### A is

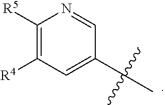

Embodiment 16. The compound of formula (I) or any one of Embodiments 1 to 6, or pharmaceutically acceptable salt thereof, wherein

### A is

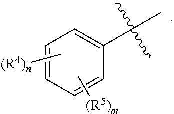

Embodiment 17. The compound of formula (I) or any one of Embodiments 1 to 6, or pharmaceutically acceptable salt thereof, wherein

### A is

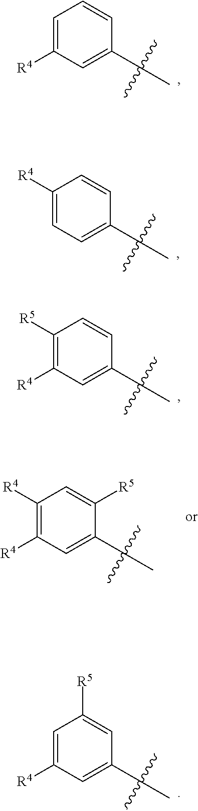

Embodiment 18. The compound of formula (I) or any one of Embodiments 1 to 6, or pharmaceutically acceptable salt thereof, wherein

### A is

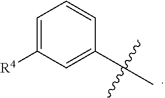

Embodiment 19. The compound of formula (I) or any one of Embodiments 1 to 6, or pharmaceutically acceptable salt thereof, wherein

### A is

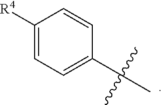

Embodiment 20. The compound of formula (I) or any one of Embodiments 1 to 6, or pharmaceutically acceptable salt thereof, wherein

### A is

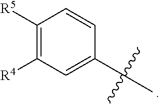

Embodiment 21. The compound of formula (I) or any one of Embodiments 1 to 6, or pharmaceutically acceptable salt thereof, wherein

### A is

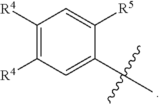

Embodiment 22. The compound of formula (I) or any one of Embodiments 1 to 6, or pharmaceutically acceptable salt thereof, wherein

### A is

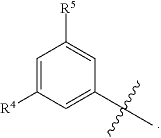

Embodiment 23. The compound of formula (I) or any one of Embodiments 1 to 6, or pharmaceutically acceptable salt thereof, wherein

### A is

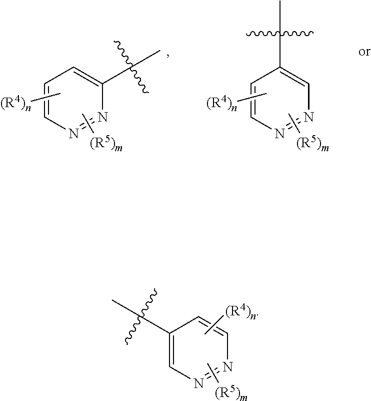

Embodiment 24. The compound of formula (I) or any one of Embodiments 1 to 6, or pharmaceutically acceptable salt thereof, wherein

### A is

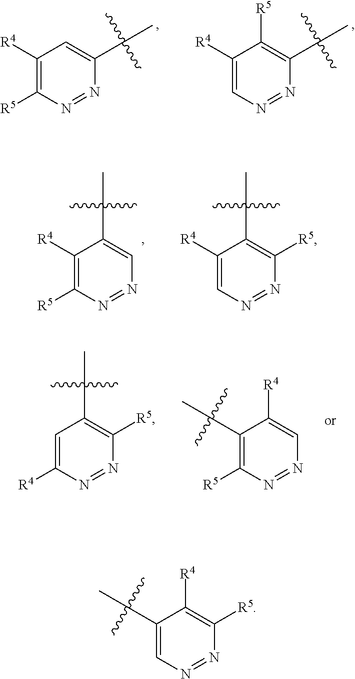

Embodiment 25. The compound of formula (I) or any one of Embodiments 1 to 6, or pharmaceutically acceptable salt thereof, wherein

### A is

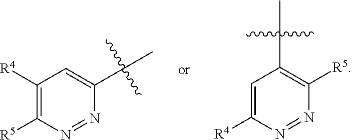

Embodiment 26. The compound of formula (I) or any one of Embodiments 1 to 6, or pharmaceutically acceptable salt thereof, wherein

### A is

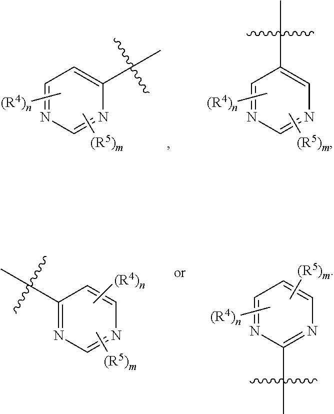

Embodiment 27. The compound of formula (I) or any one of Embodiments 1 to 6, or pharmaceutically acceptable salt thereof, wherein

### A is

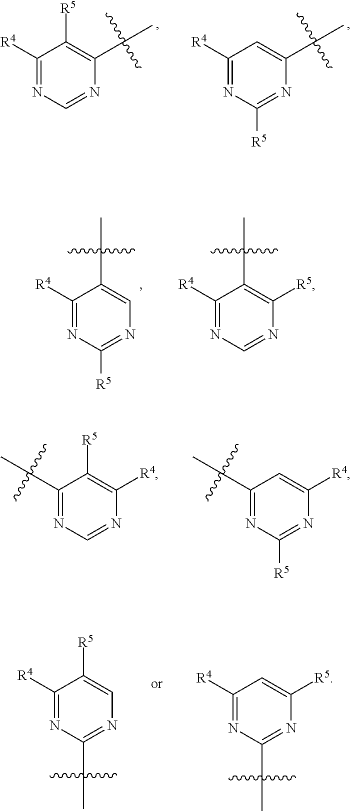

Embodiment 28. The compound of formula (I) or any one of Embodiments 1 to 6, or pharmaceutically acceptable salt thereof, wherein

### A is

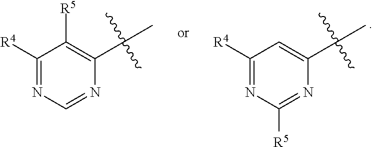

Embodiment 29. The compound of formula (I) or any one of Embodiments 1 to 6, or pharmaceutically acceptable salt thereof, wherein

### A is

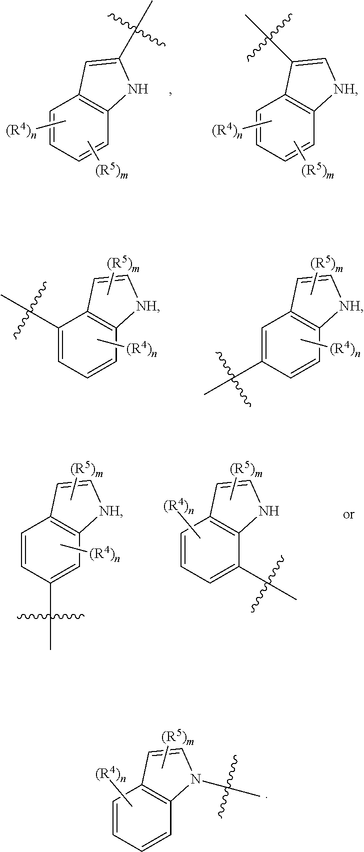

Embodiment 30. The compound of formula (I) or any one of Embodiments 1 to 6, or pharmaceutically acceptable salt thereof, wherein

### A is

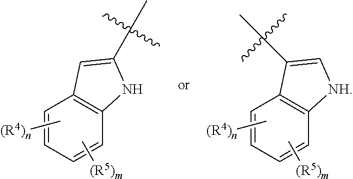

Embodiment 31. The compound of formula (I) or any one of Embodiments 1 to 6, or pharmaceutically acceptable salt thereof, wherein

### A is

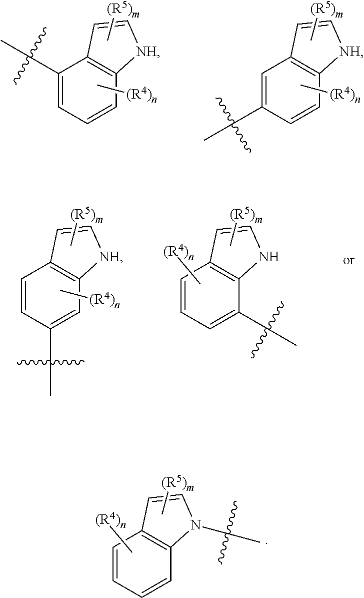

Embodiment 32. The compound of formula (I) or any one of Embodiments 1 to 6, or pharmaceutically acceptable salt thereof, wherein

### A is

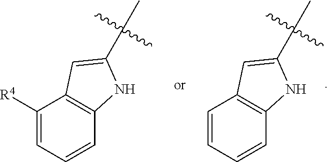

Embodiment 33. The compound of formula (I) or any one of Embodiments 1 to 6, or pharmaceutically acceptable salt thereof, wherein

### A is

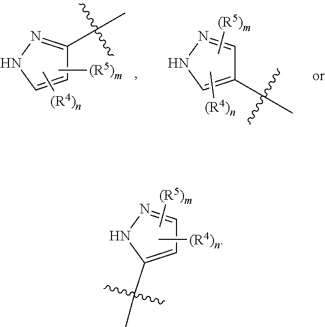

Embodiment 34. The compound of formula (I) or any one of Embodiments 1 to 6, or pharmaceutically acceptable salt thereof, wherein

### A is

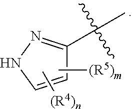

Embodiment 35. The compound of formula (I) or any one of Embodiments 1 to 6, or pharmaceutically acceptable salt thereof, wherein

### A is

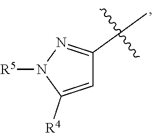

Embodiment 36. The compound of formula (I) or any one of Embodiments 1 to 35, or pharmaceutically acceptable salt thereof, wherein each of R⁴ is independently selected from halo, C₁-C₆alkyl, cyano, C₁-C₆alkoxy, C₁-C₆haloalkoxy and —C(═O)N(R¹²)₂.
Embodiment 37. The compound of formula (I) or any one of Embodiments 1 to 35, or pharmaceutically acceptable salt thereof, wherein each of R⁴ is independently selected from halo, C₁-C₆alkyl, cyano, C₁-C₆alkoxy and C₁-C₆haloalkoxy.
Embodiment 38. The compound of formula (I) or any one of Embodiments 1 to 35, or pharmaceutically acceptable salt thereof, wherein each of R⁴ is independently selected from methoxy, ethoxy, cyano, fluoro, chloro, —OCHF₂, methyl, ethyl, —C(═O)NH₂.
Embodiment 39. The compound of formula (I) or any one of Embodiments 1 to 35, or pharmaceutically acceptable salt thereof, wherein each of R⁴ is independently selected from methoxy, ethoxy, cyano, fluoro, —OCHF₂ and methyl.
Embodiment 40. The compound of formula (I) or any one of Embodiments 1 to 35, or pharmaceutically acceptable salt thereof, wherein R⁵ is selected from halo, C₁-C₆alkyl, cyano, C₁-C₆alkoxy, C₁-C₆haloalkoxy and —C(═O)N(R¹²)₂.
Embodiment 41. The compound of formula (I) or any one of Embodiments 1 to 35, or pharmaceutically acceptable salt thereof, wherein R⁵ is selected from halo, C₁-C₆alkyl, cyano, and —C(═O)N(R¹²)₂.
Embodiment 42. The compound of formula (I) or any one of Embodiments 1 to 35, or pharmaceutically acceptable salt thereof, wherein R⁵ is selected from methoxy, ethoxy, cyano, fluoro, chloro, —OCHF₂, methyl, ethyl and —C(═O)NH₂.
Embodiment 43. The compound of formula (I) or any one of Embodiments 1 to 35, or pharmaceutically acceptable salt thereof, wherein R⁵ is selected from cyano, fluoro, chloro, methyl, ethyl and —C(═O)NH₂.
Embodiment 44. The compound of formula (I) or any one of Embodiments 1 to 35, or pharmaceutically acceptable salt thereof, wherein
R⁴ is methoxy or ethoxy, and
R⁵ is cyano or fluoro.
Embodiment 45. The compound of formula (I) or any one of Embodiments 1 to 35, or pharmaceutically acceptable salt thereof, wherein
R⁴ is ethoxy, and
R⁵ is cyano.
Embodiment 46. The compound of formula (I) or any one of Embodiments 1 to 35, or pharmaceutically acceptable salt thereof, wherein
R⁴ is ethoxy, and
R⁵ is fluoro.
Embodiment 47. The compound of formula (I) or any one of Embodiments 1 to 35, or pharmaceutically acceptable salt thereof, wherein
R⁴ is methoxy, and
R⁵ is fluoro.
Embodiment 48. The compound of formula (I) or any one of Embodiments 1 to 47, or pharmaceutically acceptable salt thereof, wherein R³ is selected from H and C₁-C₆alkyl.
Embodiment 49. The compound of formula (I) or any one of Embodiments 1 to 47, or pharmaceutically acceptable salt thereof, wherein R³ is H or methyl.
Embodiment 50. The compound of formula (I) or any one of Embodiments 1 to 47, or pharmaceutically acceptable salt thereof, wherein R³ is H.
Embodiment 51. The compound of formula (I) or any one of Embodiments 1 to 47, or pharmaceutically acceptable salt thereof, wherein R³ is C₁-C₆alkyl.
Embodiment 52. The compound of formula (I) or any one of Embodiments 1 to 47, or pharmaceutically acceptable salt thereof, wherein R³ is methyl.
Embodiment 53. The compound of formula (I) or any one of Embodiments 1 to 52, or pharmaceutically acceptable salt thereof, wherein
R¹ is selected from H, halo, —R⁶, —CH₂R⁶, —CH₂NR^{1a}R^{1b}, C₁-C₆haloalkyl, a C₁-C₆alkyl substituted with 1 to 2 groups independently selected from C₁-C₆alkyl, —OR^{1a}, —S(═O)₂R¹² and —N(R¹²)₂, and a C₁-C₆haloalkyl substituted with 1 to 2 groups independently selected from —OR^{1a}, —S(═O)₂R¹² and —N(R¹²)₂,
wherein,
R^{1a} is selected from H, C₁-C₆alkyl, C₁-C₆haloalkyl, C₃-C₈cycloalkyl, and a 4-6 membered heterocycloalkyl having 1 to 2 heteroatoms independently selected from N and O, and
R^{1b} is selected from H, C₁-C₆alkyl, C₁-C₆haloalkyl, C₃-C₈cycloalkyl, and a 4-6 membered heterocycloalkyl having 1 to 2 heteroatoms independently selected from N and O.
Embodiment 54. The compound of formula (I) or any one of Embodiments 1 to 52, or pharmaceutically acceptable salt thereof, wherein
R¹ is selected from H, halo, C₁-C₆haloalkyl, a C₁-C₆alkyl substituted with 1 to 2 groups independently selected from C₁-C₆alkyl, —OH, —S(═O)₂R¹² and —N(R¹²)₂, and a C₁-C₆haloalkyl substituted with 1 to 2 groups independently selected from —OH, —S(═O)₂R¹² and —N(R¹²)₂.
Embodiment 55. The compound of formula (I) or any one of Embodiments 1 to 52, or pharmaceutically acceptable salt thereof, wherein
R¹ is selected from H, bromo, chloro, —CHF₂, a C₄-C₅alkyl substituted with an —OH group and a C₂-C₄haloalkyl substituted with 1 to 2 groups independently selected from methyl and an —OH group.
Embodiment 56. The compound of formula (I) or any one of Embodiments 1 to 52, or pharmaceutically acceptable salt thereof, wherein
R¹ is selected from H, bromo, chloro, —CHF₂, —CH(OH)CH(CH₃)₂, —CH(OH)CH(CH₃)₃, —C(CH₃)(OH)(CF₃), —CH(OH)CH(CH₃)CF₃, and —CH(OH)CF₃.
Embodiment 57. The compound of formula (I) or any one of Embodiments 1 to 52, or pharmaceutically acceptable salt thereof, wherein
R¹ is selected from —R⁶, —CH₂R⁶, and —CH₂NR^{1a}R^{1b},
wherein,
R^{1a} is selected from H, C₁-C₆alkyl, C₁-C₆haloalkyl, C₃-C₈cycloalkyl, and a 4-6 membered heterocycloalkyl having 1 to 2 heteroatoms independently selected from N and O, and
R^{1b} is selected from H, C₁-C₆alkyl, C₁-C₆haloalkyl, C₃-C₈cycloalkyl, and a 4-6 membered heterocycloalkyl having 1 to 2 heteroatoms independently selected from N and O.
Embodiment 58. The compound of formula (I) or any one of Embodiments 1 to 52, or pharmaceutically acceptable salt thereof, wherein
R¹ is —CH₂NR^{1a}R^{1b},
wherein,
R^{1a} is selected from H, C₁-C₆alkyl, C₁-C₆haloalkyl, C₃-C₈cycloalkyl, and a 4-6 membered heterocycloalkyl having 1 to 2 heteroatoms independently selected from N and O, and
R^{1b} is selected from H, C₁-C₆alkyl, C₁-C₆haloalkyl, C₃-C₈cycloalkyl, and a 4-6 membered heterocycloalkyl having 1 to 2 heteroatoms independently selected from N and O.
Embodiment 59. The compound of formula (I) or any one of Embodiments 1 to 52, or pharmaceutically acceptable salt thereof, wherein
R¹ is —CH₂NR^{1a}R^{1b},
wherein,
R^{1a} is selected from C₁-C₂alkyl, C₁-C₂haloalkyl, C₃-C₅cycloalkyl, and a 4 membered heterocycloalkyl having 1 to 2 heteroatoms independently selected from N and O, and
R^{1b} is selected from C₁-C₂alkyl, C₁-C₂haloalkyl, C₃-C₅cycloalkyl, and a 4 membered heterocycloalkyl having 1 to 2 heteroatoms independently selected from N and O.
Embodiment 60. The compound of formula (I) or any one of Embodiments 1 to 52, or pharmaceutically acceptable salt thereof, wherein
R¹ is —CH₂NR^{1a}R^{1b},
wherein,
R^{1a} is selected from methyl, —CH₂CHF₂, —CH₂CF₃, —CH₂CH₂F, cyclopropyl, and an oxetanyl, and
R^{1b} is selected from methyl, —CH₂CHF₂, —CH₂CF₃, —CH₂CH₂F, cyclopropyl, and an oxetanyl.
Embodiment 61. The compound of formula (I) or any one of Embodiments 1 to 52, or pharmaceutically acceptable salt thereof, wherein:
R¹ is —CH₂NR^{1a}R^{1b};
R^{1a} is selected from methyl, —CH₂CHF₂, —CH₂CF₃, —CH₂CH₂F, cyclopropyl, and an oxetanyl, and
R^{1b} is methyl.
Embodiment 62. The compound of formula (I) or any one of Embodiments 1 to 52, or pharmaceutically acceptable salt thereof, wherein R¹ is selected from —R⁶ and —CH₂R⁶.
Embodiment 63. The compound of formula (I) or any one of Embodiments 1 to 52, or pharmaceutically acceptable salt thereof, wherein
R¹ is —R⁶.
Embodiment 64. The compound of formula (I) or any one of Embodiments 1 to 52, or pharmaceutically acceptable salt thereof, wherein
R¹ is —CH₂R⁶.
Embodiment 65. The compound of formula (I) or any one of Embodiments 1 to 52 or Embodiments 62 to 64, or pharmaceutically acceptable salt thereof, wherein
R⁶ is selected from phenyl, pyridinonyl, tetrahydropyridinyl, pyridazinonyl, a 4-6 membered heterocycloalkyl having 1 to 2 heteroatoms independently selected from N and O, a 5-6 membered heteroaryl having 1 to 3 heteroatoms independently selected from N, O and S, a 9 to 10 membered heteroaryl having 1-3 heteroatoms independently selected from N, O and S, C₃-C₈cycloalkyl,

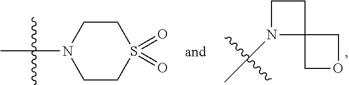 [SMILES: *N1CCS(=O)(=O)CC1 ; *N1CCC12COC2]

wherein the phenyl, pyridinonyl, pyridazinonyl, heterocycloalkyl and heteroaryl of R⁶ are optionally substituted with 1 to 3 R⁹ groups.
Embodiment 66. The compound of formula (I) or any one of Embodiments 1 to 52 or Embodiments 62 to 64, or pharmaceutically acceptable salt thereof, wherein
R⁶ is selected from phenyl, cyclopropyl. pyridinonyl, tetrahydropyridinyl, pyridazinonyl, morpholinyl, pyrrolidinyl, azetadinyl, cyclopropyl pyridyl, pyrazolyl, pyrimidinyl, imidazolyl, pyridazinyl, oxazolopyridinyl, imidazopyridinyl or triazolyl, each of which is optionally substituted with 1 to 3 R⁹ groups.
Embodiment 67. The compound of formula (I) or any one of Embodiments 1 to 52 or Embodiments 62 to 64, or pharmaceutically acceptable salt thereof, wherein
R⁶ is C₃-C₈cycloalkyl.
Embodiment 68. The compound of formula (I) or any one of Embodiments 1 to 52 or Embodiments 62 to 64, or pharmaceutically acceptable salt thereof, wherein
R⁶ is C₃-C₅cycloalkyl.
Embodiment 69. The compound of formula (I) or any one of Embodiments 1 to 52 or Embodiments 62 to 64, or pharmaceutically acceptable salt thereof, wherein
R⁶ is cyclopropyl.
Embodiment 70. The compound of formula (I) or any one of Embodiments 1 to 52 or Embodiments 62 to 64, or pharmaceutically acceptable salt thereof, wherein
R⁶ is selected from phenyl optionally substituted with 1 to 3 R⁹ groups.
Embodiment 71. The compound of formula (I) or any one of Embodiments 1 to 52 or Embodiments 62 to 64, or pharmaceutically acceptable salt thereof, wherein
R⁶ is selected from pyridinonyl, tetrahydropyridinyl and pyridazinonyl, wherein the pyridinonyl and pyridazinonyl of R⁶ are optionally substituted with 1 to 3 R⁹ groups.
Embodiment 72. The compound of formula (I) or any one of Embodiments 1 to 52 or Embodiments 62 to 64, or pharmaceutically acceptable salt thereof, wherein
R⁶ is selected from a 4-6 membered heterocycloalkyl having 1 to 2 heteroatoms independently selected from N and O,

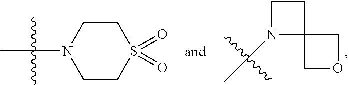 [SMILES: *N1CCS(=O)(=O)CC1 ; *N1CCC12COC2]

wherein the heterocycloalkyl of R⁶ is optionally substituted with 1 to 3 R⁹ groups.
Embodiment 73. The compound of formula (I) or any one of Embodiments 1 to 52 or Embodiments 62 to 64, or pharmaceutically acceptable salt thereof, wherein
R⁶ is morpholinyl, pyrrolidinyl or azetadinyl, each of which is optionally substituted with 1 to 3 R⁹ groups.
Embodiment 74. The compound of formula (I) or any one of Embodiments 1 to 52 or Embodiments 62 to 64, or pharmaceutically acceptable salt thereof, wherein
R⁶ is selected from a 5-6 membered heteroaryl having 1 to 3 nitrogen atoms and a 9 to 10 membered heteroaryl having 1-3 heteroatoms independently selected from N and O, wherein the heteroaryl of R⁶ is optionally substituted with 1 to 3 R⁹ groups.
Embodiment 75. The compound of formula (I) or any one of Embodiments 1 to 52 or Embodiments 62 to 64, or pharmaceutically acceptable salt thereof, wherein
R⁶ is pyridyl, pyrazolyl, pyrimidinyl, imidazolyl, pyridazinyl, oxazolopyridinyl, imidazopyridinyl, triazolyl each of which is optionally substituted with 1 to 3 R⁹ groups.
Embodiment 76. The compound of formula (I) or any one of Embodiments 1 to 52 or Embodiments 62 to 64, or pharmaceutically acceptable salt thereof, wherein
R⁶ is pyridin-3-yl, pyridin-4-yl, pyrazol-4-yl, pyrazol-5-yl pyrimidin-5-yl, imidazol-1-yl, imidazol-5-yl, pyridazin-4-yl, oxazolo[4,5-b]pyridin-6-yl, imidazo[1,2-a]pyridin-6-yl, imidazo[4,5-b]pyridin-6-yl, triazol-5-yl, triazol-4-yl and triazol-1-yl, each of which is optionally substituted with 1 to 3 R⁹ groups.
Embodiment 77. The compound of formula (I) or any one of Embodiments 1 to 52 or Embodiments 62 to 64, or pharmaceutically acceptable salt thereof, wherein
R¹ is selected from —R⁶ and —CH₂R⁶;
R⁶ is selected from azetadinyl and pyridyl, each of which is optionally substituted with 1 to 3 R⁹ groups, and
each R⁹ is independently selected from fluoro and methyl.
Embodiment 78. The compound of formula (I) or any one of Embodiments 1 to 52 or Embodiments 62 to 77, or pharmaceutically acceptable salt thereof, wherein
each R⁹ is independently selected from C₁-C₆alkyl, C₁-C₆alkoxy, C₁-C₆haloalkyl, C₃-C₈cycloalkyl, halo, cyano, —N(R¹²)₂, —OH, —C(═O)R¹², —S(═O)₂R¹² and C₁-C₆alkyl substituted with cyano, —N(R¹²)₂ or 1 to 2 —OH groups.
Embodiment 79. The compound of formula (I) or any one of Embodiments 1 to 52 or Embodiments 62 to 77, or pharmaceutically acceptable salt thereof, wherein
each R⁸ is independently selected from C₁-C₂alkyl, C₁-C₂alkoxy, C₁-C₂haloalkyl, C₃-C₅cycloalkyl, halo, cyano, —N(R¹²)₂, —OH, —C(═O)R¹², —S(═O)₂R¹² and C₁-C₃alkyl substituted with cyano, —N(R¹²)₂ or 1 to 2 —OH groups.
Embodiment 80. The compound of formula (I) or any one of Embodiments 1 to 52 or Embodiments 62 to 77, or pharmaceutically acceptable salt thereof, wherein
each R⁸ is independently selected from fluoro, chloro, methyl, ethyl, methoxy, cyano, cyclopropyl, —OH, —CF₃, —CHF₂, —CH₂OH, —CHOHCH₃, —CH₂CH₂OH, —C(CH₃)₂OH, —NH₂, —C(═O)CH₃, —CH₂CN, —CH₂N(CH₃)₂, —NHCH₃ and S(═O)₂CH₃.
Embodiment 81. The compound of formula (I) or any one of Embodiments 1 to 80, or pharmaceutically acceptable salt thereof, wherein R² is selected from —(CH₂)R⁷, —(CH₂)₂R⁸ and —(CH₂)₂NR^{2a}R^{2b}.
Embodiment 82. The compound of formula (I) or any one of Embodiments 1 to 80, or pharmaceutically acceptable salt thereof, wherein R² is —(CH₂)₂NR^{2a}R^{2b}; and wherein,
R^{2a} is selected from H, C₁-C₆alkyl, C₁-C₆haloalkyl, C₃-C₈cycloalkyl, a 4-6 membered heterocycloalkyl having 1 to 2 heteroatoms independently selected from N and O, and a C₁-C₆alkyl substituted with 1 to 2 groups selected from —OH groups and C₁-C₆alkoxy, and
R^{2b} is selected from H, C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy.
Embodiment 83. The compound of formula (I) or any one of Embodiments 1 to 80, or pharmaceutically acceptable salt thereof, wherein R² is —(CH₂)₂NR^{2a}R^{2b}; and wherein,
R^{2a} is selected from C₁-C₆alkyl, C₁-C₆haloalkyl, C₃-C₈cycloalkyl, a 4-6 membered heterocycloalkyl having 1 to 2 heteroatoms independently selected from N and O, and a C₁-C₆alkyl substituted with 1 to 2 groups selected from —OH groups and C₁-C₆alkoxy, and
R^{2b} is selected from C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy.
Embodiment 84. The compound of formula (I) or any one of Embodiments 1 to 80, or pharmaceutically acceptable salt thereof, wherein R² is —(CH₂)₂NR^{2a}R^{2b}; and wherein,
R^{2a} is selected from C₁-C₂alkyl, C₁-C₃haloalkyl, C₃-C₅cycloalkyl, a 4 membered heterocycloalkyl having 1 to 2 heteroatoms independently selected from N and O, and a C₁-C₃alkyl substituted with 1 to 2 groups selected from —OH groups and C₁-C₃alkoxy, and
R^{2b} is selected from C₁-C₂alkyl, C₁-C₃haloalkyl, C₃-C₅cycloalkyl, a 4 membered heterocycloalkyl having 1 to 2 heteroatoms independently selected from N and O, and C₁-C₃alkyl substituted with 1 to 2 groups selected from —OH groups and C₁-C₃alkoxy.
Embodiment 85. The compound of formula (I) or any one of Embodiments 1 to 80, or pharmaceutically acceptable salt thereof, wherein R² is —(CH₂)₂NR^{2a}R^{2b}; and wherein,
R^{2a} is selected from H, methyl, ethyl, cyclopropyl, oxetanyl, —CH₂CH₂F, —CH₂CH₂OH, —CH₂CH₂OCH₃ and —CH₂CH(OH)CH₃, and
R^{2b} is selected from H, methyl, ethyl, cyclopropyl, oxetanyl, —CH₂CH₂F, —CH₂CH₂OH, —CH₂CH₂OCH₃ and —CH₂CH(OH)CH₃.
Embodiment 86. The compound of formula (I) or any one of Embodiments 1 to 80, or pharmaceutically acceptable salt thereof, wherein R² is —(CH₂)₂NR^{2a}R^{2b}; and wherein,
R^{2a} is selected from methyl, ethyl, cyclopropyl, oxetanyl, —CH₂CH₂F, —CH₂CH₂OH, —CH₂CH₂OCH₃ and —CH₂CH(OH)CH₃, and
R^{2b} is methyl.
Embodiment 87. The compound of formula (I) or any one of Embodiments 1 to 80, or pharmaceutically acceptable salt thereof, wherein R² is —(CH₂)₂NR^{2a}R^{2b}; and wherein,
R^{2a} is ethyl, and
R^{2b} is methyl.
Embodiment 88. The compound of formula (I) or any one of Embodiments 1 to 80, or pharmaceutically acceptable salt thereof, wherein R² is —(CH₂)R⁷.
Embodiment 89. The compound of formula (I) or any one of Embodiments 1 to 80 or Embodiment 88, or pharmaceutically acceptable salt thereof, wherein R⁷ is a 5-6 membered heteroaryl having 1 to 3 nitrogen atoms optionally substituted with 1 to 3 R¹⁰ groups.
Embodiment 90. The compound of formula (I) or any one of Embodiments 1 to 80 or Embodiments 88 to 89, or pharmaceutically acceptable salt thereof, wherein R⁷ is a 5 membered heteroaryl having 1 to 3 nitrogen atoms, optionally substituted with 1 to 3 R¹⁰ groups.
Embodiment 91. The compound of formula (I) or any one of Embodiments 1 to 80 or Embodiments 88 to 90, or pharmaceutically acceptable salt thereof, wherein R⁷ is imidazolyl, optionally substituted with 1 to 3 R¹⁰ groups.
Embodiment 92. The compound of formula (I) or any one of Embodiments 1 to 80 or Embodiments 88 to 91, or pharmaceutically acceptable salt thereof, wherein R⁷ is imidazole-1-yl, optionally substituted with 1 to 3 R¹⁰ groups.
Embodiment 93. The compound of formula (I) or any one of Embodiments 1 to 80 or Embodiments 88 to 92, or pharmaceutically acceptable salt thereof, wherein
each R¹⁰ is independently selected from C₁-C₆alkyl, C₁-C₆alkoxy, C₁-C₆haloalkyl, C₃-C₈cycloalkyl, halo, cyano, —N(R¹²)₂, —OH, and C₁-C₆alkyl substituted with cyano, —N(R¹²)₂ or 1 to 2 —OH groups.
Embodiment 94. The compound of formula (I) or any one of Embodiments 1 to 80 or Embodiments 86 to 92, or pharmaceutically acceptable salt thereof, wherein
each R¹⁰ is independently selected from C₁-C₆alkyl, C₁-C₆alkoxy, halo, —N(R¹²)₂, —OH, and C₁-C₆alkyl substituted with 1 to 2 —OH groups.
Embodiment 95. The compound of formula (I) or any one of Embodiments 1 to 80 or Embodiments 88 to 92, or pharmaceutically acceptable salt thereof, wherein
each R¹⁰ is independently selected from fluoro, methyl, methoxy, —OH, —CH₂OH, and —NHCH₃.
Embodiment 96. The compound of formula (I) or any one of Embodiments 1 to 80 or Embodiments 88 to 95, or or a pharmaceutically acceptable salt thereof, wherein
R² is —(CH₂)R⁷, and
R⁷ is imidazolyl substituted with —NHCH₃.
Embodiment 97. The compound of formula (I) or any one of Embodiments 1 to 80, or pharmaceutically acceptable salt thereof, wherein R² is —(CH₂)₂R⁸.
Embodiment 98. The compound of formula (I) or any one Embodiments 1 to 80 or Embodiment 97, or pharmaceutically acceptable salt thereof, wherein R⁸ is a 4-6 membered heterocycloalkyl having 1 to 2 heteroatoms independently selected from N and O, optionally substituted with 1 to 3 R¹¹ groups.
Embodiment 99. The compound of formula (I) or any one of Embodiments 1 to 80 or Embodiments 97 to 98, or pharmaceutically acceptable salt thereof, wherein R⁸ is a 4-5 membered heterocycloalkyl having 1 to 2 heteroatoms independently selected from N and O, optionally substituted with 1 to 3 R¹¹ groups.
Embodiment 100. The compound of formula (I) or any one of Embodiments 1 to 80 or Embodiments 97 to 99, or pharmaceutically acceptable salt thereof, wherein R⁸ is a 4 membered heterocycloalkyl having a heteroatom i selected from N and O, optionally substituted with 1 to 3 R¹¹ groups.
Embodiment 101. The compound of formula (I) or any one of Embodiments 1 to 80 or Embodiments 97 to 100, or pharmaceutically acceptable salt thereof, wherein R⁷ is azetidinyl, pyrrolidinyl, optionally substituted with 1 to 3 R¹¹ groups.
Embodiment 102. The compound of formula (I) or any one of Embodiments 1 to 80 or Embodiments 97 to 101, or pharmaceutically acceptable salt thereof, wherein R⁷ is azetidin-1-yl, pyrrolidin-1-yl, optionally substituted with 1 to 3 R¹¹ groups.
Embodiment 103. The compound of formula (I) or any one of Embodiments 1 to 80 or Embodiments 97 to 102, or pharmaceutically acceptable salt thereof, wherein
each R¹¹ is independently selected from C₁-C₆alkyl, C₁-C₆alkoxy, C₁-C₆haloalkyl, C₃-C₈cycloalkyl, halo, cyano, —N(R¹²)₂, —OH, and C₁-C₆alkyl substituted with cyano, —N(R¹²)₂ or 1 to 2 —OH groups.
Embodiment 104. The compound of formula (I) or any one of Embodiments 1 to 80 or Embodiments 97 to 103, or pharmaceutically acceptable salt thereof, wherein
each R¹¹ is independently selected from C₁-C₆alkyl, C₁-C₆alkoxy, halo, —N(R¹²)₂, —OH, and C₁-C₆alkyl substituted with 1 to 2 —OH groups.
Embodiment 105. The compound of formula (I) or any one of Embodiments 1 to 80 or Embodiments 97 to 104, or pharmaceutically acceptable salt thereof, wherein
each R¹¹ is independently selected from fluoro, methyl, methoxy, —OH, —CH₂OH, and —NHCH₃.
Embodiment 106. The compound of formula (I) or any one of Embodiments 1 to 80 or Embodiments 97 to 105, or or a pharmaceutically acceptable salt thereof, wherein
R² is —(CH₂)₂R⁸;
R⁸ is azetidinyl, pyrrolidinyl or imidazolyl, each of which is optionally substituted with 1 to 3 R¹¹ groups, and
each R¹¹ is independently selected from fluoro, methyl, methoxy, —OH, —CH₂OH, and —NHCH₃.
Embodiment 107. The compound of formula (I) or any one of Embodiments 1 to 106, pharmaceutically acceptable salt thereof, wherein
each R¹² is independently selected from H and C₁-C₆alkyl.
Embodiment 108. The compound of formula (I) or any one of Embodiments 1 to 106, pharmaceutically acceptable salt thereof, wherein
each R¹² is independently selected from H and C₁-C₃alkyl.
Embodiment 109. The compound of formula (I) or any one of Embodiments 1 to 106, pharmaceutically acceptable salt thereof, wherein
each R¹² is independently selected from H, methyl and ethyl.
Embodiment 110. The compound of formula (I) or any one of Embodiments 1 to 106, pharmaceutically acceptable salt thereof, wherein
each R¹² is independently selected from H and methyl.
Embodiment 111. The compound of formula (I) or any one of Embodiments 1 to 110, pharmaceutically acceptable salt thereof, wherein m is 0 or 1.
Embodiment 112. The compound of formula (I) or any one of Embodiments 1 to 110, pharmaceutically acceptable salt thereof, wherein m is 0.
Embodiment 113. The compound of formula (I) or any one of Embodiments 1 to 110, pharmaceutically acceptable salt thereof, wherein m is 1.
Embodiment 114. The compound of formula (I) or any one of Embodiments 1 to 113, pharmaceutically acceptable salt thereof, wherein n is 0, 1 or 2.
Embodiment 115. The compound of formula (I) or any one of Embodiments 1 to 113, pharmaceutically acceptable salt thereof, wherein n is 0.
Embodiment 116. The compound of formula (I) or any one of Embodiments 1 to 113, pharmaceutically acceptable salt thereof, wherein n is 1.
Embodiment 117. The compound of formula (I) or any one of Embodiments 1 to 113, pharmaceutically acceptable salt thereof, wherein n is 2.
Embodiment 118. The compound of formula (I) or any one of Embodiments 1 to 117, pharmaceutically acceptable salt thereof, having the structure of formula (Ie), formula (If) or formula (Ig), or pharmaceutically acceptable salt thereof,

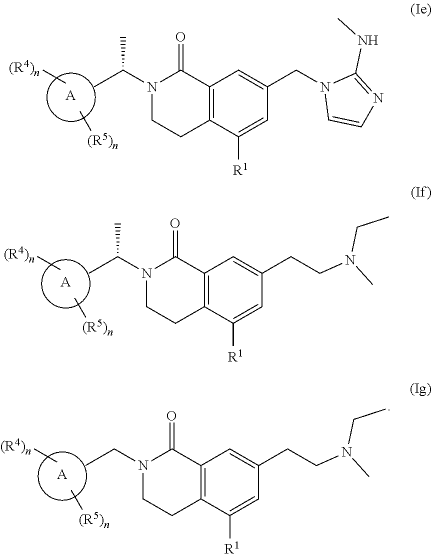

Embodiment 119. The compound of formula (I) or Embodiment 118, or pharmaceutically acceptable salt thereof, having the structure of formula (Ie), formula (If) or formula (Ig), or pharmaceutically acceptable salt thereof, wherein:
A is selected from

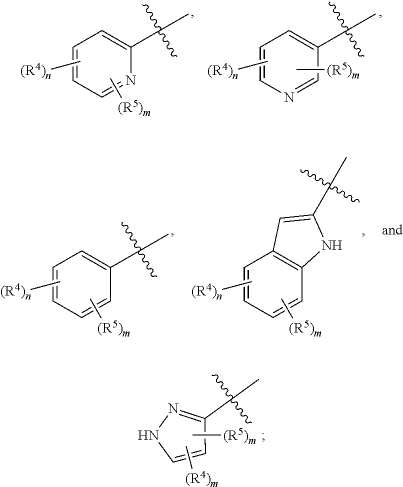

R¹ is selected from H, halo, —R⁶, —CH₂R⁶, —CH₂NR^{1a}R^{1b}, C₁-C₆haloalkyl, a C₁-C₆alkyl substituted with 1 to 2 groups independently selected from —OR^{1a} and —N(R¹²)₂, and a C₁-C₆haloalkyl substituted with 1 to 2 groups independently selected from C₁-C₆alkyl, —OR^{1a} and —N(R¹²)₂;
   wherein,
      R^{1a} is selected from H, C₁-C₆alkyl, C₁-C₆haloalkyl, C₃-C₈cycloalkyl, and a 4-6 membered heterocycloalkyl having 1 to 2 heteroatoms independently selected from N and O, and
      R^{1b} is selected from H, C₁-C₆alkyl, C₁-C₆haloalkyl, C₃-C₈cycloalkyl, and a 4-6 membered heterocycloalkyl having 1 to 2 heteroatoms independently selected from N and O;
R² is selected from —(CH₂)R⁷, —(CH₂)₂R⁸ and —(CH₂)₂NR^{2a}R^{2b};
   wherein,
      R^{2a} is selected from C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy, and
      R^{2b} is selected from C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy;
each of R⁴ is independently selected from halo, C₁-C₆alkyl, cyano, C₁-C₆alkoxy, C₁-C₆haloalkoxy and —C(═O)N(R¹²)₂;
R⁵ selected from halo, C₁-C₆alkyl, cyano, C₁-C₆alkoxy, C₁-C₆haloalkoxy and —C(═O)N(R¹²)₂;
R⁶ is selected from phenyl, pyridinonyl, tetrahydropyridinyl, pyridazinonyl, a 4-6 membered heterocycloalkyl having 1 to 2 heteroatoms independently selected from N and O, a 5-6 membered heteroaryl having 1 to 3 heteroatoms independently selected from N, O and S, a 9 to 10 membered heteroaryl having 1-3 heteroatoms independently selected from N, O and S, C₃-C₈cycloalkyl and

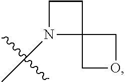 [SMILES: *N1CCC12COC2]

wherein the phenyl, pyridinonyl, pyridazinonyl, heterocycloalkyl and heteroaryl of R⁶ are optionally substituted with 1 to 3 R⁹ groups, and wherein each R⁹ is independently selected from C₁-C₆alkyl, C₁-C₆alkoxy, C₁-C₆haloalkyl, C₃-C₈cycloalkyl, halo, cyano, —N(R¹²)₂, —OH, —C(═O)R¹², —S(═O)₂R¹² and a C₁-C₆alkyl substituted with cyano, —N(R¹²)₂ or 1 to 2 —OH groups;
R⁷ is a 5-6 membered heteroaryl having 1 to 3 nitrogen atoms, optionally substituted with 1 to 3 R¹⁰ groups, and wherein each R¹⁰ is independently selected from C₁-C₆alkyl, C₁-C₆alkoxy, C₁-C₆haloalkyl, C₃-C₈cycloalkyl, halo, cyano, —N(R¹²)₂, —OH, and a C₁-C₆alkyl substituted with cyano, —N(R¹²)₂ or 1 to 2 —OH groups;
R⁸ is a 4-6 membered heterocycloalkyl having 1 to 2 heteroatoms independently selected from N and O, optionally substituted with 1 to 3 R¹¹ groups, and wherein each R¹ is independently selected from C₁-C₆alkyl, C₁-C₆alkoxy, C₁-C₆haloalkyl, C₃-C₈cycloalkyl, halo, cyano, —N(R¹²)₂, —OH, and a C₁-C₆alkyl substituted with cyano, —N(R¹²)₂ or 1 to 2 —OH groups;
each R¹² is independently selected from H and C₁-C₆alkyl;
m is 0 or 1 and
n is 0, 1 or 2.
   Embodiment 120. The compound of formula (I) or Embodiments 118 to 119, or pharmaceutically acceptable salt thereof, wherein:
A is

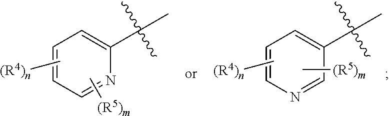

R¹ is selected from H, halo, —R⁶, —CH₂R⁶, —CH₂NR^{1a}R^{1b}, C₁-C₆haloalkyl, a C₁-C₆alkyl substituted with 1 to 2 groups independently selected from —OR^{1a} and —N(R¹²)₂, and a C₁-C₆haloalkyl substituted with 1 to 2 groups independently selected from C₁-C₆alkyl, —OR^{1a} and —N(R¹²)₂;
   wherein,
      R^{1a} is selected from H, C₁-C₆alkyl, C₁-C₆haloalkyl, C₃-C₈cycloalkyl, and a 4-6 membered heterocycloalkyl having 1 to 2 heteroatoms independently selected from N and O, and
      R^{1b} is selected from H, C₁-C₆alkyl, C₁-C₆haloalkyl, C₃-C₈cycloalkyl, and a 4-6 membered heterocycloalkyl having 1 to 2 heteroatoms independently selected from N and O;
R² is selected from —(CH₂)R⁷ and —(CH₂)₂NR^{2a}R^{2b};
   wherein,
      R^{2a} is selected from C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy, and
      R^{2b} is selected from C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy;
each of R⁴ is independently selected from halo, C₁-C₆alkyl, cyano, C₁-C₆alkoxy, C₁-C₆haloalkoxy and —C(═O)N(R¹²)₂;
R⁵ selected from halo, C₁-C₆alkyl, cyano, C₁-C₆alkoxy, C₁-C₆haloalkoxy and —C(═O)N(R¹²)₂;
R⁶ is selected from phenyl, pyridinonyl, tetrahydropyridinyl, pyridazinonyl, a 4-6 membered heterocycloalkyl having 1 to 2 heteroatoms independently selected from N and O, a 5-6 membered heteroaryl having 1 to 3 heteroatoms independently selected from N, O and S, a 9 to 10 membered heteroaryl having 1-3 heteroatoms independently selected from N, O and S, C₃-C₈cycloalkyl and

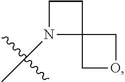 [SMILES: *N1CCC12COC2]

wherein the phenyl, pyridinonyl, pyridazinonyl, heterocycloalkyl and heteroaryl of R⁶ are optionally substituted with 1 to 3 R⁹ groups, and wherein each R⁹ is independently selected from C₁-C₆alkyl, C₁-C₆alkoxy, C₁-C₆haloalkyl, C₃-C₈cycloalkyl, halo, cyano, —N(R¹²)₂, —OH, —C(═O)R¹², —S(═O)₂R¹² and a C₁-C₆alkyl substituted with cyano, —N(R¹²)₂ or 1 to 2 —OH groups;
R⁷ is a 5-6 membered heteroaryl having 1 to 3 nitrogen atoms, optionally substituted with 1 to 3 R¹⁰ groups, and wherein each R¹⁰ is independently selected from C₁-C₆alkyl, C₁-C₆alkoxy, C₁-C₆haloalkyl, C₃-C₈cycloalkyl, halo, cyano, —N(R¹²)₂, —OH, and a C₁-C₆alkyl substituted with cyano, —N(R¹²)₂ or 1 to 2 —OH groups;
each R¹² is independently selected from H and C₁-C₆alkyl;
m is 0 or 1 and
n is 0, 1 or 2.
   Embodiment 121. The compound of formula (I) or Embodiments 118 to 119, or pharmaceutically acceptable salt thereof, wherein:
A is

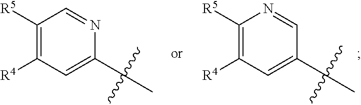

R¹ is selected from H, —R⁶ and —CH₂NR^{1a}R^{1b};
   wherein,
      R^{1a} is C₁-C₆alkyl, and
      R^{1b} is C₁-C₆alkyl;
R² is selected from —(CH₂)R⁷ and —(CH₂)₂NR^{2a}R^{2b};
   wherein,
      R^{2a} is C₁-C₆alkyl and
      R^{2b} is C₁-C₆alkyl;
R⁴ is C₁-C₆alkoxy;
R⁵ is halo or cyano;
R⁶ is pyridinonyl or a 5-6 membered heteroaryl having 1 to 3 heteroatoms independently selected from N, O and S, wherein the pyridinonyl and heteroaryl of R⁶ are substituted with 1 to 3 R⁹ groups, and wherein each R⁹ is independently selected from C₁-C₆alkyl, C₁-C₆haloalkyl, halo and —N(R¹²)₂;
R⁷ is a 5-6 membered heteroaryl having 1 to 3 nitrogen atoms, substituted with 1 to 3 R¹⁰ groups, and wherein each R¹⁰ is independently selected from C₁-C₆alkyl and —N(R¹²)₂, and
each R¹² is independently selected from H and C₁-C₆alkyl.
   Embodiment 122. The compound of formula (I) or Embodiments 118 to 119, or pharmaceutically acceptable salt thereof, wherein:
A is

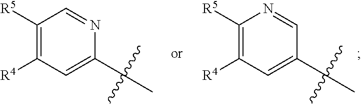

R¹ is selected from H, —R⁶ and —CH₂NR^{1a}R^{1b};
   wherein,
      R^{1a} is methyl, and
      R^{1b} is methyl;
R² is selected from —(CH₂)R⁷ and —(CH₂)₂NR^{2a}R^{2b};
   wherein,
      R^{2a} is ethyl and
      R^{2b} is methyl;
R⁴ is methoxy or ethoxy;
R⁵ is fluoro or cyano;
R⁶ is a pyridyl substituted with 1 to 3 groups independently selected from fluoro, methyl and —NH₂; a pyrazolyl substituted with 1 to 3 methyl; or a pyridinonyl substituted with 1 to 3 groups independently selected from methyl and —CF₃;
R⁷ is imidazolyl substituted with methyl or —N(R¹²)₂,
and
each R¹² is independently selected from H and methyl.
   Embodiment 123. A compound selected from:
6-(1-(5-bromo-7-((2-(methylamino)-1H-imidazol-1-yl)methyl)-1-oxo-3,4 dihydroisoquinolin-2(1H)-yl)ethyl)-4-ethoxynicotinonitrile;
5-((5-bromo-7-((2-(methylamino)-1H-imidazol-1-yl)methyl)-1-oxo-3,4-dihydroisoquinolin-2(1H)-yl)methyl)-3-ethoxypicolinonitrile;
(S)-6-(1-(5-chloro-7-((2-(methylamino)-1H-imidazol-1-yl)methyl)-1-oxo-3,4-dihydroisoquinolin-2(1H)-yl)ethyl)-4-ethoxynicotinonitrile;
(R)-5-(1-(5-chloro-7-((2-(methylamino)-1H-imidazol-1-yl)methyl)-1-oxo-3,4-dihydroisoquinolin-2(1H)-yl)ethyl)-3-ethoxypicolinonitrile;
(S)-5-chloro-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
5-((5-chloro-7-((2-(methylamino)-1H-imidazol-1-yl)methyl)-1-oxo-3,4-dihydroisoquinolin-2(1H)-yl)methyl)-3-ethoxypicolinonitrile;
(S)-2-(1-(5-fluoro-4-methoxypyridin-2-yl)ethyl)-7-((2-(methylamino)-1H-imidazol-1-yl)methyl)-5-(morpholinomethyl)-3,4-dihydroisoquinolin-1(2H)-one;
(S)-2-(1-(6-fluoro-5-methoxypyridin-3-yl)ethyl)-7-((2-(methylamino)-1H-imidazol-1-yl)methyl)-5-(morpholinomethyl)-3,4-dihydroisoquinolin-1(2H)-one;
4-ethoxy-6-((S)-1-(5-(((S)-3-fluoropyrrolidin-1-yl)methyl)-7-((2-(methylamino)-1H-imidazol-1-yl)methyl)-1-oxo-3,4-dihydroisoquinolin-2(1H)-yl)ethyl)nicotinonitrile;
(S)-2-(1-(4-ethoxy-5-fluoropyridin-2-yl)ethyl)-5-((methyl(oxetan-3-yl)amino)methyl)-7-((2-(methylamino)-1H-imidazol-1-yl)methyl)-3,4-dihydroisoquinolin-1(2H)-one;
4-ethoxy-6-((S)-1-(5-(((R)-3-fluoropyrrolidin-1-yl)methyl)-7-((2-(methylamino)-1H-imidazol-1-yl)methyl)-1-oxo-3,4-dihydroisoquinolin-2(1H)-yl)ethyl)nicotinonitrile;
(S)-4-ethoxy-6-(1-(7-((2-(methylamino)-1H-imidazol-1-yl)methyl)-5-(morpholinomethyl)-1-oxo-3,4-dihydroisoquinolin-2(1H)-yl)ethyl)nicotinonitrile;
(S)-2-(1-(5-fluoro-4-methoxypyridin-2-yl)ethyl)-5-((methyl(oxetan-3-yl)amino)methyl)-7-((2-(methylamino)-1H-imidazol-1-yl)methyl)-3,4-dihydroisoquinolin-1(2H)-one;
2-((S)-1-(4-ethoxy-5-fluoropyridin-2-yl)ethyl)-5-(((R)-3-fluoropyrrolidin-1-yl)methyl)-7-((2-(methylamino)-1H-imidazol-1-yl)methyl)-3,4-dihydroisoquinolin-1(2H)-one;
(S)-5-((cyclopropyl(methyl)amino)methyl)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
2-((S)-1-(5-fluoro-4-methoxypyridin-2-yl)ethyl)-5-(((R)-3-fluoropyrrolidin-1-yl)methyl)-7-((2-(methylamino)-1H-imidazol-1-yl)methyl)-3,4-dihydroisoquinolin-1(2H)-one;
(S)-5-((6-oxa-1-azaspiro[3.3]heptan-1-yl)methyl)-2-(1-(5-fluoro-4-methoxypyridin-2-yl)ethyl)-7-((2-(methylamino)-1H-imidazol-1-yl)methyl)-3,4-dihydroisoquinolin-1(2H)-one;
(S)-6-(1-(5-((cyclopropyl(methyl)amino)methyl)-7-((2-(methylamino)-1H-imidazol-1-yl)methyl)-1-oxo-3,4-dihydroisoquinolin-2(1H)-yl)ethyl)-4-ethoxynicotinonitrile;
(S)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(R)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(S)-2-(1-(5-chloro-4-ethoxypyridin-2-yl)ethyl)-5-((dimethylamino)methyl)-7-((2-(methylamino)-1H-imidazol-1-yl)methyl)-3,4-dihydroisoquinolin-1(2H)-one;
(S)-5-((cyclopropyl(methyl)amino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(S)-2-(1-(5-chloro-4-ethoxypyridin-2-yl)ethyl)-5-((cyclopropyl(methyl)amino)methyl)-7-((2-(methylamino)-1H-imidazol-1-yl)methyl)-3,4-dihydroisoquinolin-1(2H)-one;
(S)-2-(1-(5-chloro-4-ethoxypyridin-2-yl)ethyl)-7-((2-(methylamino)-1H-imidazol-1-yl)methyl)-5-(morpholinomethyl)-3,4-dihydroisoquinolin-1(2H)-one;
5-((5-((dimethylamino)methyl)-7-((2-(methylamino)-1H-imidazol-1-yl)methyl)-1-oxo-3,4-dihydroisoquinolin-2(1H)-yl)methyl)-3-ethoxypicolinonitrile;
3-ethoxy-5-((7-((2-(methylamino)-1H-imidazol-1-yl)methyl)-5-((3-methylazetidin-1-yl)methyl)-1-oxo-3,4-dihydroisoquinolin-2(1H)-yl)methyl)picolinonitrile;
5-((5-((cyclopropyl(methyl)amino)methyl)-7-((2-(methylamino)-1H-imidazol-1-yl)methyl)-1-oxo-3,4-dihydroisoquinolin-2(1H)-yl)methyl)-3-ethoxypicolinonitrile;
(S)-2-(1-(4-ethoxy-5-fluoropyridin-2-yl)ethyl)-7-((2-(methylamino)-1H-imidazol-1-yl)methyl)-5-(morpholinomethyl)-3,4-dihydroisoquinolin-1(2H)-one;
(S)-4-ethoxy-6-(1-(5-((3-fluoroazetidin-1-yl)methyl)-7-((2-(methylamino)-1H-imidazol-1-yl)methyl)-1-oxo-3,4-dihydroisoquinolin-2(1H)-yl)ethyl)nicotinonitrile;
(S)-5-((dimethylamino)methyl)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
2-((5-fluoro-4-methoxypyridin-2-yl)methyl)-5-((3-fluoroazetidin-1-yl)methyl)-7-((2-(methylamino)-1H-imidazol-1-yl)methyl)-3,4-dihydroisoquinolin-1(2H)-one;
(S)-2-(1-(5-fluoro-4-methoxypyridin-2-yl)ethyl)-5-((3-methoxyazetidin-1-yl)methyl)-7-((2-(methylamino)-1H-imidazol-1-yl)methyl)-3,4-dihydroisoquinolin-1(2H)-one;
(S)-5-(1-(5-((dimethylamino)methyl)-7-((2-(methylamino)-1H-imidazol-1-yl)methyl)-1-oxo-3,4-dihydroisoquinolin-2(1H)-yl)ethyl)-3-ethoxypicolinonitrile;
2-((6-fluoro-5-methoxypyridin-3-yl)methyl)-5-((3-fluoroazetidin-1-yl)methyl)-7-((2-(methylamino)-1H-imidazol-1-yl)methyl)-3,4-dihydroisoquinolin-1(2H)-one;
(S)-5-((3,3-difluoroazetidin-1-yl)methyl)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
(S)-5-(((2,2-difluoroethyl)(methyl)amino)methyl)-2-(1-(5-fluoro-4-methoxypyridin-2-yl)ethyl)-7-((2-(methylamino)-1H-imidazol-1-yl)methyl)-3,4-dihydroisoquinolin-1(2H)-one;
2-((S)-1-(5-fluoro-4-methoxypyridin-2-yl)ethyl)-5-(((R)-3-methoxypyrrolidin-1-yl)methyl)-7-((2-(methylamino)-1H-imidazol-1-yl)methyl)-3,4-dihydroisoquinolin-1(2H)-one;
2-((S)-1-(4-ethoxy-5-fluoropyridin-2-yl)ethyl)-5-(((S)-3-fluoropyrrolidin-1-yl)methyl)-7-((2-(methylamino)-1H-imidazol-1-yl)methyl)-3,4-dihydroisoquinolin-1(2H)-one;
5-((3,3-difluoroazetidin-1-yl)methyl)-2-((5-fluoro-4-methoxypyridin-2-yl)methyl)-7-((2-(methylamino)-1H-imidazol-1-yl)methyl)-3,4-dihydroisoquinolin-1(2H)-one;
(S)-2-(1-(4-ethoxy-5-fluoropyridin-2-yl)ethyl)-5-((3-fluoroazetidin-1-yl)methyl)-7-((2-(methylamino)-1H-imidazol-1-yl)methyl)-3,4-dihydroisoquinolin-1(2H)-one;
(S)-6-(1-(5-((3,3-difluoroazetidin-1-yl)methyl)-7-((2-(methylamino)-1H-imidazol-1-yl)methyl)-1-oxo-3,4-dihydroisoquinolin-2(1H)-yl)ethyl)-4-ethoxynicotinonitrile;
(S)-3-ethoxy-5-(1-(5-((3-fluoroazetidin-1-yl)methyl)-7-((2-(methylamino)-1H-imidazol-1-yl)methyl)-1-oxo-3,4-dihydroisoquinolin-2(1H)-yl)ethyl)picolinonitrile;
(S)-2-(1-(5-fluoro-4-methoxypyridin-2-yl)ethyl)-5-((3-fluoroazetidin-1-yl)methyl)-7-((2-(methylamino)-1H-imidazol-1-yl)methyl)-3,4-dihydroisoquinolin-1(2H)-one;
(S)-2-(1-(6-fluoro-5-methoxypyridin-3-yl)ethyl)-5-((3-fluoroazetidin-1-yl)methyl)-7-((2-(methylamino)-1H-imidazol-1-yl)methyl)-3,4-dihydroisoquinolin-1(2H)-one;
2-((S)-1-(5-fluoro-4-methoxypyridin-2-yl)ethyl)-5-(((S)-3-fluoropyrrolidin-1-yl)methyl)-7-((2-(methylamino)-1H-imidazol-1-yl)methyl)-3,4-dihydroisoquinolin-1(2H)-one;
(S)-6-(1-(5-((dimethylamino)methyl)-7-((2-(methylamino)-1H-imidazol-1-yl)methyl)-1-oxo-3,4-dihydroisoquinolin-2(1H)-yl)ethyl)-4-methoxynicotinonitrile;
5-((5-((3,3-difluoroazetidin-1-yl)methyl)-7-((2-(methylamino)-1H-imidazol-1-yl)methyl)-1-oxo-3,4-dihydroisoquinolin-2(1H)-yl)methyl)-3-ethoxypicolinonitrile;
(S)-2-(1-(5-fluoro-4-methoxypyridin-2-yl)ethyl)-5-((methyl(2,2,2-trifluoroethyl)amino)methyl)-7-((2-(methylamino)-1H-imidazol-1-yl)methyl)-3,4-dihydroisoquinolin-1(2H)-one;
(S)-5-((3,3-difluoroazetidin-1-yl)methyl)-2-(1-(5-fluoro-4-methoxypyridin-2-yl)ethyl)-7-((2-(methylamino)-1H-imidazol-1-yl)methyl)-3,4-dihydroisoquinolin-1(2H)-one;
(S)-2-(1-(5-fluoro-4-methoxypyridin-2-yl)ethyl)-5-(((2-fluoroethyl)(methyl)amino)methyl)-7-((2-(methylamino)-1H-imidazol-1-yl)methyl)-3,4-dihydroisoquinolin-1(2H)-one;
(S)-6-(1-(5-((dimethylamino)methyl)-7-((2-(methylamino)-1H-imidazol-1-yl)methyl)-1-oxo-3,4-dihydroisoquinolin-2(1H)-yl)ethyl)-4-ethoxynicotinonitrile;
(S)-2-(1-(5-fluoro-4-methoxypyridin-2-yl)ethyl)-5-((3-hydroxyazetidin-1-yl)methyl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-(3-methoxybenzyl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-(4-fluoro-3-methoxybenzyl)-7-((2-(methylamino)-1H-imidazol-1-yl)methyl)-3,4-dihydroisoquinolin-1(2H)-one;
2-(4-fluoro-3-methoxybenzyl)-5-(1-methyl-3-(trifluoromethyl)-1H-pyrazol-4-yl)-7-((2-(methylamino)-1H-imidazol-1-yl)methyl)-3,4-dihydroisoquinolin-1(2H)-one;
2-(4-fluoro-3-methoxybenzyl)-5-(3-methyl-1H-pyrazol-4-yl)-7-((2-(methylamino)-1H-imidazol-1-yl)methyl)-3,4-dihydroisoquinolin-1(2H)-one;
(S)-5-(2-chloro-5-(hydroxymethyl)pyridin-3-yl)-2-(1-(4-fluoro-3-methoxyphenyl)ethyl)-7-((2-(methylamino)-1H-imidazol-1-yl)methyl)-3,4-dihydroisoquinolin-1(2H)-one;
(R)-5-(2-chloro-5-(hydroxymethyl)pyridin-3-yl)-2-(1-(4-fluoro-3-methoxyphenyl)ethyl)-7-((2-(methylamino)-1H-imidazol-1-yl)methyl)-3,4-dihydroisoquinolin-1(2H)-one;
5-(1,3-dimethyl-1H-pyrazol-4-yl)-2-(4-fluoro-3-methoxybenzyl)-7-((2-(methylamino)-1H-imidazol-1-yl)methyl)-3,4-dihydroisoquinolin-1(2H)-one;
(S)-2-(1-(5-fluoro-4-methoxypyridin-2-yl)ethyl)-7-((2-(methylamino)-1H-imidazol-1-yl)methyl)-5-(pyrimidin-5-yl)-3,4-dihydroisoquinolin-1(2H)-one;
2-((5-fluoro-4-methoxypyridin-2-yl)methyl)-5-(1-methyl-1H-pyrazol-5-yl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-(2,4-difluoro-5-methoxybenzyl)-7-((2-(methylamino)-1H-imidazol-1-yl)methyl)-3,4-dihydroisoquinolin-1(2H)-one;
(S)-5-(1,3-dimethyl-1H-pyrazol-4-yl)-2-(1-(5-fluoro-4-methoxypyridin-2-yl)ethyl)-7-((2-(methylamino)-1H-imidazol-1-yl)methyl)-3,4-dihydroisoquinolin-1(2H)-one;
(S)-2-(1-(5-fluoro-4-methoxypyridin-2-yl)ethyl)-7-((2-(methylamino)-1H-imidazol-1-yl)methyl)-5-(1H-pyrazol-5-yl)-3,4-dihydroisoquinolin-1(2H)-one;
2-((5-fluoro-4-methoxypyridin-2-yl)methyl)-5-(1-methyl-3-(trifluoromethyl)-1H-pyrazol-4-yl)-7-((2-(methylamino)-1H-imidazol-1-yl)methyl)-3,4-dihydroisoquinolin-1(2H)-one;
5-(2-chloro-5-(hydroxymethyl)phenyl)-2-(4-fluoro-3-methoxybenzyl)-7-((2-(methylamino)-1H-imidazol-1-yl)methyl)-3,4-dihydroisoquinolin-1(2H)-one;
5-(3-(difluoromethyl)-1-methyl-1H-pyrazol-4-yl)-2-(4-fluoro-3-methoxybenzyl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-(3-chloro-5-fluorobenzyl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-((5-fluoro-4-methoxypyridin-2-yl)methyl)-7-((2-(methylamino)-1H-imidazol-1-yl)methyl)-3,4-dihydroisoquinolin-1(2H)-one;
5-((5-(2-chloro-5-(hydroxymethyl)pyridin-3-yl)-7-((2-(methylamino)-1H-imidazol-1-yl)methyl)-1-oxo-3,4-dihydroisoquinolin-2(1H)-yl)methyl)-2-fluorobenzonitrile;
5-(2-chloro-5-(hydroxymethyl)pyridin-3-yl)-2-(3,4-difluorobenzyl)-7-((2-(methylamino)-1H-imidazol-1-yl)methyl)-3,4-dihydroisoquinolin-1(2H)-one;
(R)-5-(1,3-dimethyl-1H-pyrazol-4-yl)-2-(1-(5-fluoro-4-methoxypyridin-2-yl)ethyl)-7-((2-(methylamino)-1H-imidazol-1-yl)methyl)-3,4-dihydroisoquinolin-1(2H)-one;
2-(4-fluoro-3-methoxybenzyl)-5-(1-methyl-1H-pyrazol-5-yl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-(3-(difluoromethoxy)benzyl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-(3-(difluoromethoxy)-4-fluorobenzyl)-7-((2-(methylamino)-1H-imidazol-1-yl)methyl)-3,4-dihydroisoquinolin-1(2H)-one;
(R)-5-(2-chloro-5-(1-hydroxyethyl)pyridin-3-yl)-2-(4-fluoro-3-methoxybenzyl)-7-((2-(methylamino)-1H-imidazol-1-yl)methyl)-3,4-dihydroisoquinolin-1(2H)-one;
(S)-5-(2-chloro-5-(1-hydroxyethyl)pyridin-3-yl)-2-(4-fluoro-3-methoxybenzyl)-7-((2-(methylamino)-1H-imidazol-1-yl)methyl)-3,4-dihydroisoquinolin-1(2H)-one;
4-(2-(4-fluoro-3-methoxybenzyl)-7-((2-(methylamino)-1H-imidazol-1-yl)methyl)-1-oxo-1,2,3,4-tetrahydroisoquinolin-5-yl)-1-methyl-1H-pyrazole-3-carbonitrile;
(S)-5-cyclopropyl-2-(1-(6-fluoro-5-methoxypyridin-3-yl)ethyl)-7-((2-(methylamino)-1H-imidazol-1-yl)methyl)-3,4-dihydroisoquinolin-1(2H)-one;
5-cyclopropyl-2-((6-fluoro-5-methoxypyridin-3-yl)methyl)-7-((2-(methylamino)-1H-imidazol-1-yl)methyl)-3,4-dihydroisoquinolin-1(2H)-one;
(S)-5-(1-(5-cyclopropyl-7-((2-(methylamino)-1H-imidazol-1-yl)methyl)-1-oxo-3,4-dihydroisoquinolin-2(1H)-yl)ethyl)-3-ethoxypicolinonitrile;
(S)-5-cyclopropyl-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(R)-6-(1-(5-cyclopropyl-7-((2-(methylamino)-1H-imidazol-1-yl)methyl)-1-oxo-3,4-dihydroisoquinolin-2(1H)-yl)ethyl)-4-ethoxynicotinonitrile;
(S)-6-(1-(5-cyclopropyl-7-((2-(methylamino)-1H-imidazol-1-yl)methyl)-1-oxo-3,4-dihydroisoquinolin-2(1H)-yl)ethyl)-4-ethoxynicotinonitrile;
5-((5-cyclopropyl-7-((2-(methylamino)-1H-imidazol-1-yl)methyl)-1-oxo-3,4-dihydroisoquinolin-2(1H)-yl)methyl)-3-ethoxypicolinonitrile;
(S)-5-cyclopropyl-2-(1-(5-fluoro-4-methoxypyridin-2-yl)ethyl)-7-((2-(methylamino)-1H-imidazol-1-yl)methyl)-3,4-dihydroisoquinolin-1(2H)-one;
5-cyclopropyl-2-((1-ethyl-5-methoxy-1H-pyrazol-3-yl)methyl)-7-((2-(methylamino)-1H-imidazol-1-yl)methyl)-3,4-dihydroisoquinolin-1(2H)-one;
5-cyclopropyl-2-((5-ethoxy-1-methyl-1H-pyrazol-3-yl)methyl)-7-((2-(methylamino)-1H-imidazol-1-yl)methyl)-3,4-dihydroisoquinolin-1(2H)-one;
(S)-2-(1-(4-ethoxy-5-fluoropyridin-2-yl)ethyl)-7-((2-(methylamino)-1H-imidazol-1-yl)methyl)-5-(pyrrolidin-1-yl)-3,4-dihydroisoquinolin-1(2H)-one;
(S)-2-(1-(5-fluoro-4-methoxypyridin-2-yl)ethyl)-7-((2-(methylamino)-1H-imidazol-1-yl)methyl)-5-(pyrrolidin-1-yl)-3,4-dihydroisoquinolin-1(2H)-one;
(S)-2-(1-(5-fluoro-4-methoxypyridin-2-yl)ethyl)-5-(3-methoxyazetidin-1-yl)-7-((2-(methylamino)-1H-imidazol-1-yl)methyl)-3,4-dihydroisoquinolin-1(2H)-one;
(S)-5-(difluoromethyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(S)-5-(difluoromethyl)-2-(1-(5-fluoro-4-methoxypyridin-2-yl)ethyl)-7-((2-(methylamino)-1H-imidazol-1-yl)methyl)-3,4-dihydroisoquinolin-1(2H)-one;
(S)-5-(difluoromethyl)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
(S)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
(R)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
2-(1-(6-fluoro-5-methoxypyridin-3-yl)ethyl)-7-((2-(methylamino)-1H-imidazol-1-yl)methyl)-3,4-dihydroisoquinolin-1(2H)-one;
(S)-2-(1-(6-chloro-5-ethoxypyridin-3-yl)ethyl)-7-((2-(methylamino)-1H-imidazol-1-yl)methyl)-3,4-dihydroisoquinolin-1(2H)-one;
3-ethoxy-5-((7-((2-(methylamino)-1H-imidazol-1-yl)methyl)-1-oxo-3,4-dihydroisoquinolin-2(1H)-yl)methyl)picolinonitrile;
(S)-3-ethoxy-5-(1-(7-((2-(methylamino)-1H-imidazol-1-yl)methyl)-1-oxo-3,4-dihydroisoquinolin-2(1H)-yl)ethyl)picolinonitrile;
5-(2-chloro-5-(hydroxymethyl)pyridin-3-yl)-2-(3-(difluoromethoxy)benzyl)-7-((2-(methylamino)-1H-imidazol-1-yl)methyl)isoquinolin-1(2H)-one;
5-bromo-2-(4-fluoro-3-methoxybenzyl)-7-((2-(methylamino)-1H-imidazol-1-yl)methyl)isoquinolin-1(2H)-one;
5-(2-chloro-5-(hydroxymethyl)pyridin-3-yl)-2-(3-methoxybenzyl)-7-((2-(methylamino)-1H-imidazol-1-yl)methyl)isoquinolin-1(2H)-one;
5-(2-chloro-5-(hydroxymethyl)pyridin-3-yl)-2-(4-fluoro-3-methoxybenzyl)-7-((2-(methylamino)-1H-imidazol-1-yl)methyl)isoquinolin-1(2H)-one;
(S)-5-(2-chloro-5-(hydroxymethyl)pyridin-3-yl)-2-(1-(3-methoxyphenyl)ethyl)-7-((2-(methylamino)-1H-imidazol-1-yl)methyl)isoquinolin-1(2H)-one;
(R)-5-(2-chloro-5-(hydroxymethyl)pyridin-3-yl)-2-(1-(3-methoxyphenyl)ethyl)-7-((2-(methylamino)-1H-imidazol-1-yl)methyl)isoquinolin-1(2H)-one;
5-(2-chloro-5-(hydroxymethyl)pyridin-3-yl)-2-(3-fluoro-4-methoxybenzyl)-7-((2-(methylamino)-1H-imidazol-1-yl)methyl)isoquinolin-1(2H)-one;
5-(2-chloro-5-(hydroxymethyl)pyridin-3-yl)-2-(4-methoxybenzyl)-7-((2-(methylamino)-1H-imidazol-1-yl)methyl)isoquinolin-1(2H)-one;
5-(2-chloro-5-(1-hydroxyethyl)pyridin-3-yl)-2-(4-fluoro-3-methoxybenzyl)-7-((2-(methylamino)-1H-imidazol-1-yl)methyl)isoquinolin-1(2H)-one;
5-(2-chloro-5-(hydroxymethyl)pyridin-3-yl)-2-((4-methyl-1H-indol-2-yl)methyl)-7-((2-(methylamino)-1H-imidazol-1-yl)methyl)isoquinolin-1(2H)-one;
2-((1H-indol-2-yl)methyl)-5-(2-chloro-5-(hydroxymethyl)pyridin-3-yl)-7-((2-(methylamino)-1H-imidazol-1-yl)methyl)isoquinolin-1(2H)-one;
2-(1-(6-fluoro-5-methoxypyridin-3-yl)ethyl)-7-((2-(methylamino)-1H-imidazol-1-yl)methyl)phthalazin-1(2H)-one;
3-(1-(6-fluoro-5-methoxypyridin-3-yl)ethyl)-6-((2-(methylamino)-1H-imidazol-1-yl)methyl)quinazolin-4(3H)-one;
(S)-4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile;
(R)-4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile;
4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile;
6-((5-(2-chloro-5-methylpyridin-3-yl)-7-(2-(ethyl(methyl)amino)ethyl)-1-oxo-3,4-dihydroisoquinolin-2(1H)-yl)methyl)-4-ethoxynicotinonitrile;
(S)-5-(1,3-dimethyl-1H-pyrazol-4-yl)-7-(2-(ethyl(methyl)amino)ethyl)-2-(1-(5-fluoro-4-methoxypyridin-2-yl)ethyl)-3,4-dihydroisoquinolin-1(2H)-one;
(S)-6-(1-(5-(2,5-dimethylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(1-(2-hydroxyethyl)-3-methyl-1H-pyrazol-4-yl)-1-oxo-3,4-dihydroisoquinolin-2(1H)-yl)ethyl)nicotinonitrile;
(S)-4-ethoxy-6-(1-(7-(2-(ethyl(methyl)amino)ethyl)-5-(3-methyl-1H-pyrazol-4-yl)-1-oxo-3,4-dihydroisoquinolin-2(1H)-yl)ethyl)nicotinonitrile;
(S)-4-ethoxy-6-(1-(7-(2-(ethyl(methyl)amino)ethyl)-5-(2-fluoro-3-methylpyridin-4-yl)-1-oxo-3,4-dihydroisoquinolin-2(1H)-yl)ethyl)nicotinonitrile;
(S)-6-(1-(5-(2-chloro-5-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(2-(difluoromethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-5-(2-chloro-5-(hydroxymethyl)pyridin-3-yl)-2-(1-(4-ethoxy-5-fluoropyridin-2-yl)ethyl)-7-(2-(ethyl(methyl)amino)ethyl)-3,4-dihydroisoquinolin-1(2H)-one;
(S)-2-(1-(5-chloro-4-ethoxypyridin-2-yl)ethyl)-5-(1,3-dimethyl-1H-pyrazol-4-yl)-7-(2-(ethyl(methyl)amino)ethyl)-3,4-dihydroisoquinolin-1(2H)-one;
(S)-6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(R)-6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-5-(1,3-dimethyl-1H-pyrazol-4-yl)-7-(2-(ethyl(methyl)amino)ethyl)-2-(1-(6-fluoro-5-methoxypyridin-3-yl)ethyl)-3,4-dihydroisoquinolin-1(2H)-one;
(S)-6-(1-(5-(2-chloro-5-(hydroxymethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-1-oxo-5-(1,4,5-trimethyl-6-oxo-1,6-dihydropyridin-3-yl)-3,4-dihydroisoquinolin-2(1H)-yl)ethyl)nicotinonitrile;
(S)-6-(1-(5-(3-(difluoromethyl)-1-methyl-1H-pyrazol-4-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(1-ethyl-3-methyl-1H-pyrazol-4-yl)-1-oxo-3,4-dihydroisoquinolin-2(1H)-yl)ethyl)nicotinonitrile;
(S)-6-(1-(5-(2-(difluoromethyl)-5-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
6-((5-(1,3-dimethyl-1H-pyrazol-4-yl)-7-(2-(ethyl(methyl)amino)ethyl)-1-oxo-3,4-dihydroisoquinolin-2(1H)-yl)methyl)-4-ethoxynicotinonitrile;
4-ethoxy-6-((7-(2-(ethyl(methyl)amino)ethyl)-5-(6-fluoro-2-methylpyridin-3-yl)-1-oxo-3,4-dihydroisoquinolin-2(1H)-yl)methyl)nicotinonitrile;
(S)-6-(1-(5-(1,4-dimethyl-6-oxo-1,6-dihydropyridin-3-yl)-7-(2-(ethyl(methyl)amino)ethyl)-1-oxo-3,4-dihydroisoquinolin-2(1H)-yl)ethyl)-4 ethoxynicotinonitrile;
2-((4-ethoxy-5-fluoropyridin-2-yl)methyl)-7-(2-(ethyl(methyl)amino)ethyl)-5-(6-fluoro-2-methylpyridin-3-yl)-3,4-dihydroisoquinolin-1(2H)-one;
6-((5-(5-chloro-2-methylpyridin-4-yl)-7-(2-(ethyl(methyl)amino)ethyl)-1-oxo-3,4-dihydroisoquinolin-2(1H)-yl)methyl)-4-ethoxynicotinonitrile;
(S)-6-(1-(5-(1,3-dimethyl-1H-pyrazol-4-yl)-7-(2-(ethyl(methyl)amino)ethyl)-1-oxo-3,4-dihydroisoquinolin-2(1H)-yl)ethyl)-4-ethoxynicotinonitrile;
6-((5-(1,4-dimethyl-6-oxo-1,6-dihydropyridin-3-yl)-7-(2-(ethyl(methyl)amino)ethyl)-1-oxo-3,4-dihydroisoquinolin-2(1H)-yl)methyl)-4-ethoxynicotinonitrile;
6-(1-(5-(5-chloro-2-methoxypyridin-4-yl)-7-(2-(ethyl(methyl)amino)ethyl)-1-oxo-3,4-dihydroisoquinolin-2(1H)-yl)ethyl)-4-ethoxynicotinonitrile;
7-(2-(ethyl(methyl)amino)ethyl)-5-(6-fluoro-2-methylpyridin-3-yl)-2-((6-fluoro-5-methoxypyridin-3-yl)methyl)-3,4-dihydroisoquinolin-1(2H)-one;
(S)-6-(1-(5-(5-amino-2-(trifluoromethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(2-methylpyridin-3-yl)-1-oxo-3,4-dihydroisoquinolin-2(1H)-yl)ethyl)nicotinonitrile;
6-((5-(2,5-dimethylpyridin-3-yl)-7-(2-(ethyl(methyl)amino)ethyl)-1-oxo-3,4-dihydroisoquinolin-2(1H)-yl)methyl)-4-ethoxynicotinonitrile;
4-ethoxy-6-(1-(7-(2-(ethyl(methyl)amino)ethyl)-5-(2-fluoro-3-methylpyridin-4-yl)-1-oxo-3,4-dihydroisoquinolin-2(1H)-yl)ethyl)nicotinonitrile;
(S)-4-ethoxy-6-(1-(7-(2-(ethyl(methyl)amino)ethyl)-5-(5-fluoro-2-methylpyridin-4-yl)-1-oxo-3,4-dihydroisoquinolin-2(1H)-yl)ethyl)nicotinonitrile;
4-ethoxy-6-(1-(7-(2-(ethyl(methyl)amino)ethyl)-5-(2-fluoro-5-methylpyridin-4-yl)-1-oxo-3,4-dihydroisoquinolin-2(1H)-yl)ethyl)nicotinonitrile;
6-((5-(2-(difluoromethyl)-5-methylpyridin-3-yl)-7-(2-(ethyl(methyl)amino)ethyl)-1-oxo-3,4-dihydroisoquinolin-2(1H)-yl)methyl)-4-ethoxynicotinonitrile;
(S)-5-(1,3-dimethyl-1H-pyrazol-4-yl)-2-(1-(4-ethoxy-5-fluoropyridin-2-yl)ethyl)-7-(2-(ethyl(methyl)amino)ethyl)-3,4-dihydroisoquinolin-1(2H)-one;
5-(3-(difluoromethyl)-1-methyl-1H-pyrazol-4-yl)-7-(2-(ethyl(methyl)amino)ethyl)-2-(1-(6-fluoro-5-methoxypyridin-3-yl)ethyl)-3,4-dihydroisoquinolin-1(2H)-one;
6-(1-(5-(6-chloro-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-(1-(7-(2-(ethyl(methyl)amino)ethyl)-5-(1-methyl-3-(trifluoromethyl)-1H-pyrazol-4-yl)-1-oxo-3,4-dihydroisoquinolin-2(1H)-yl)ethyl)nicotinonitrile;
(S)-6-(1-(5-(4-(difluoromethyl)-1-methyl-6-oxo-1,6-dihydropyridin-3-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-(1-(7-(2-(ethyl(methyl)amino)ethyl)-5-(5-hydroxy-2-methylpyridin-3-yl)-1-oxo-3,4-dihydroisoquinolin-2(1H)-yl)ethyl)nicotinonitrile;
4-ethoxy-6-(1-(7-(2-(ethyl(methyl)amino)ethyl)-5-(2-ethylpyridin-3-yl)-1-oxo-3,4-dihydroisoquinolin-2(1H)-yl)ethyl)nicotinonitrile;
(S)-6-(1-(5-(3-chloropyridin-4-yl)-7-(2-(ethyl(methyl)amino)ethyl)-1-oxo-3,4-dihydroisoquinolin-2(1H)-yl)ethyl)-4-ethoxynicotinonitrile;
6-(1-(5-(2-chloro-5-(1-hydroxyethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-((7-(2-(ethyl(methyl)amino)ethyl)-5-(5-fluoro-2-methylpyridin-4-yl)-1-oxo-3,4-dihydroisoquinolin-2(1H)-yl)methyl)nicotinonitrile;
6-((5-(6-amino-2-methylpyridin-3-yl)-7-(2-(ethyl(methyl)amino)ethyl)-1-oxo-3,4-dihydroisoquinolin-2(1H)-yl)methyl)-4-ethoxynicotinonitrile;
(R)-6-(1-(5-(2,5-dimethylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(1-methyl-1H-imidazol-5-yl)-1-oxo-3,4-dihydroisoquinolin-2(1H)-yl)ethyl)nicotinonitrile;
6-(1-(5-cyclopropyl-7-(2-(ethyl(methyl)amino)ethyl)-1-oxo-3,4-dihydroisoquinolin-2(1H)-yl)ethyl)-4-ethoxynicotinonitrile;
4-ethoxy-6-(1-(7-(2-(ethyl(methyl)amino)ethyl)-1-oxo-5-(1,2,3,6-tetrahydropyridin-4-yl)-3,4-dihydroisoquinolin-2(1H)-yl)ethyl)nicotinonitrile;
(S)-6-(1-(5-(5-cyclopropyl-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(1,3-dimethyl-2-oxo-1,2-dihydropyridin-4-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(2-amino-5-methylpyridin-4-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(2,5-dimethylpyridin-4-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-((7-(2-(ethyl(methyl)amino)ethyl)-5-(2-methoxy-5-methylpyridin-4-yl)-1-oxo-3,4-dihydroisoquinolin-2(1H)-yl)methyl)nicotinonitrile;
(S)-6-(1-(5-(3-(difluoromethyl)-1-ethyl-1H-pyrazol-4-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(5-chloro-2-methylpyridin-4-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(5-methoxy-2-methylpyridin-3-yl)-1-oxo-3,4-dihydroisoquinolin-2(1H)-yl)ethyl)nicotinonitrile;
6-(1-(5-(3,6-dichloropyridazin-4-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(5-fluoro-1,4-dimethyl-6-oxo-1,6-dihydropyridin-3-yl)-1-oxo-3,4-dihydroisoquinolin-2(1H)-yl)ethyl)nicotinonitrile;
(S)-6-(1-(5-(5-chloro-1,4-dimethyl-6-oxo-1,6-dihydropyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(1-ethyl-4-methyl-6-oxo-1,6-dihydropyridin-3-yl)-1-oxo-3,4-dihydroisoquinolin-2(1H)-yl)ethyl)nicotinonitrile;
(S)-6-(1-(5-(5-(difluoromethyl)-2-methylpyridin-4-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(3,6-dimethylpyridazin-4-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(4-chloro-1-(difluoromethyl)-6-oxo-1,6-dihydropyridin-3-yl)-7-(2-(ethyl(methyl)amino)ethyl)-1-oxo-3,4-dihydroisoquinolin-2(1H)-yl)ethyl)-4 ethoxynicotinonitrile;
(S)-6-(1-(5-(4-chloro-1-methyl-6-oxo-1,6-dihydropyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-1-oxo-5-(6-oxo-4-(trifluoromethyl)-1,6-dihydropyridin-3-yl)-3,4-dihydroisoquinolin-2(1H)-yl)ethyl)nicotinonitrile;
(S)-6-(1-(5-(5-(difluoromethyl)-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(6-amino-2-chloropyridin-3-yl)-7-(2-(ethyl(methyl)amino)ethyl)-1-oxo-3,4-dihydroisoquinolin-2(1H)-yl)ethyl)-4-ethoxynicotinonitrile;
6-((5-(2,5-dimethylpyridin-4-yl)-7-(2-(ethyl(methyl)amino)ethyl)-1-oxo-3,4-dihydroisoquinolin-2(1H)-yl)methyl)-4-ethoxynicotinonitrile;
6-((5-(2-amino-5-methylpyridin-4-yl)-7-(2-(ethyl(methyl)amino)ethyl)-1-oxo-3,4-dihydroisoquinolin-2(1H)-yl)methyl)-4-ethoxynicotinonitrile;
(S)-4-ethoxy-6-(1-(7-(2-(ethyl(methyl)amino)ethyl)-5-(5-methyloxazolo[4,5-b]pyridin-6-yl)-1-oxo-3,4-dihydroisoquinolin-2(1H)-yl)ethyl)nicotinonitrile;
(S)-4-ethoxy-6-(1-(7-(2-(ethyl(methyl)amino)ethyl)-5-(1-methyl-6-oxo-4-(trifluoromethyl)-1,6-dihydropyridin-3-yl)-1-oxo-3,4-dihydroisoquinolin-2(1H)-yl)ethyl)nicotinonitrile;
(S)-6-(1-(5-(2-amino-5-chloropyridin-4-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(2-(2-hydroxypropan-2-yl)-5-methylpyridin-4-yl)-1-oxo-3,4-dihydroisoquinolin-2(1H)-yl)ethyl)nicotinonitrile;
(S)-6-(1-(5-(5-acetyl-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-((1S)-1-(7-(2-(ethyl(methyl)amino)ethyl)-5-(5-(1-hydroxyethyl)-2-methylpyridin-3-yl)-1-oxo-3,4-dihydroisoquinolin-2(1H)-yl)ethyl)nicotinonitrile;
(S)-6-(1-(5-(5-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(2-methoxy-5-methylpyridin-4-yl)-1-oxo-3,4-dihydroisoquinolin-2(1H)-yl)ethyl)nicotinonitrile;
6-((5-(3,6-dimethylpyridazin-4-yl)-7-(2-(ethyl(methyl)amino)ethyl)-1-oxo-3,4-dihydroisoquinolin-2(1H)-yl)methyl)-4-ethoxynicotinonitrile;
(S)-4-ethoxy-6-(1-(7-(2-(ethyl(methyl)amino)ethyl)-5-(7-methylimidazo[1,2-a]pyridin-6-yl)-1-oxo-3,4-dihydroisoquinolin-2(1H)-yl)ethyl)nicotinonitrile;
4-ethoxy-6-((1S)-1-(7-(2-(ethyl(methyl)amino)ethyl)-5-(4-methoxy-2-methylpyridin-3-yl)-1-oxo-3,4-dihydroisoquinolin-2(1H)-yl)ethyl)nicotinonitrile;
(S)-6-(1-(5-(5-(cyanomethyl)-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(4-ethyl-1-methyl-6-oxo-1,6-dihydropyridin-3-yl)-1-oxo-3,4-dihydroisoquinolin-2(1H)-yl)ethyl)nicotinonitrile;
(S)-6-(1-(5-(3-chloro-1-methyl-2-oxo-1,2-dihydropyridin-4-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(1-methyl-2-oxo-3-(trifluoromethyl)-1,2-dihydropyridin-4-yl)-1-oxo-3,4-dihydroisoquinolin-2(1H)-yl)ethyl)nicotinonitrile;
4-ethoxy-6-((7-(2-(ethyl(methyl)amino)ethyl)-5-(1-methyl-6-oxo-4-(trifluoromethyl)-1,6-dihydropyridin-3-yl)-1-oxo-3,4-dihydroisoquinolin-2(1H)-yl)methyl)nicotinonitrile;
6-(1-(5-(5-chloro-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(6-amino-2-(trifluoromethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
(R)-6-(1-(5-(6-amino-2-(trifluoromethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
6-(1-(5-(6-amino-2-(difluoromethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
6-((5-(5-amino-2-methylpyridin-3-yl)-7-(2-(ethyl(methyl)amino)ethyl)-1-oxo-3,4-dihydroisoquinolin-2(1H)-yl)methyl)-4-ethoxynicotinonitrile;
(S)-6-(1-(5-(2-amino-5-(trifluoromethyl)pyridin-4-yl)-7-(2-(ethyl(methyl)amino)ethyl)-1-oxo-3,4-dihydroisoquinolin-2(1H)-yl)ethyl)-4-ethoxynicotinonitrile;
6-(1-(5-(6-chloro-3-methylpyridazin-4-yl)-7-(2-(ethyl(methyl)amino)ethyl)-1-oxo-3,4-dihydroisoquinolin-2(1H)-yl)ethyl)-4-ethoxynicotinonitrile;
(S)-2-(1-(4-ethoxy-5-fluoropyridin-2-yl)ethyl)-7-(2-(ethylmethyl)amino)ethyl)-5-(1-methyl-6-oxo-4-(trifluoromethyl)-1,6-dihydropyridin-3-yl)-3,4-dihydroisoquinolin-1(2H)-one;
6-(1-(5-(1,3-dimethyl-1H-pyrazol-5-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-(1-(7-(2-(ethyl(methyl)amino)ethyl)-5-(5-methyl-1H-imidazo[4,5-b]pyridin-6-yl)-1-oxo-3,4-dihydroisoquinolin-2(1H)-yl)ethyl)nicotinonitrile;
4-ethoxy-6-(1-(7-(2-(ethyl(methyl)amino)ethyl)-1-oxo-5-(pyrrolidin-1-yl)-3,4-dihydroisoquinolin-2(1H)-yl)ethyl)nicotinonitrile;
4-ethoxy-6-(1-(7-(2-(ethyl(methyl)amino)ethyl)-5-(1H-imidazol-1-yl)-1-oxo-3,4-dihydroisoquinolin-2(1H)-yl)ethyl)nicotinonitrile;
4-ethoxy-6-(1-(7-(2-(ethyl(methyl)amino)ethyl)-1-oxo-5-(1H-pyrazol-1-yl)-3,4-dihydroisoquinolin-2(1H)-yl)ethyl)nicotinonitrile;
4-ethoxy-6-((S)-1-(7-(2-(ethyl(methyl)amino)ethyl)-5-((S)-3-hydroxypyrrolidin-1-yl)-1-oxo-3,4-dihydroisoquinolin-2(1H)-yl)ethyl)nicotinonitrile;
5-(3,6-dimethylpyridazin-4-yl)-2-(1-(4-ethoxy-5-methylpyridin-2-yl)ethyl)-7-(2-(ethyl(methyl)amino)ethyl)-3,4-dihydroisoquinolin-1(2H)-one;
5-(3,6-dimethylpyridazin-4-yl)-2-((4-ethoxy-5-methylpyridin-2-yl)methyl)-7-(2-(ethyl(methyl)amino)ethyl)-3,4-dihydroisoquinolin-1(2H)-one;
6-((S)-1-(5-(3-(difluoromethyl)-1-methyl-1H-pyrazol-4-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
6-((S)-1-(5-(5-amino-2-(trifluoromethyl)pyridin-3-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
6-((S)-1-(5-(4-chloro-1-methyl-6-oxo-1,6-dihydropyridin-3-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
6-((S)-1-(5-(1,4-dimethyl-6-oxo-1,6-dihydropyridin-3-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
4-ethoxy-6-((S)-1-(5-(6-fluoro-2-methylpyridin-3-yl)-7-(2-((2R,3R)-3-hydroxy-2-methylazetidin-1-yl)ethyl)-1-oxo-3,4-dihydroisoquinolin-2(1H)-yl)ethyl)nicotinonitrile;
6-((S)-1-(5-(5-(difluoromethyl)-2-methylpyridin-3-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
6-((S)-1-(5-(6-amino-2-methylpyridin-3-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
6-((S)-1-(5-(2,5-dimethylpyridin-3-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
6-((S)-1-(5-(2,5-dimethylpyridin-4-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
4-ethoxy-6-((5-(6-fluoro-2-methylpyridin-3-yl)-7-(2-((2R,3R)-3-hydroxy-2-methylazetidin-1-yl)ethyl)-1-oxo-3,4-dihydroisoquinolin-2(1H)-yl)methyl)nicotinonitrile;
6-((R)-1-(5-(2,5-dimethylpyridin-4-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
4-ethoxy-6-((7-(2-((2R,3R)-3-hydroxy-2-methylazetidin-1-yl)ethyl)-5-(1-methyl-6-oxo-4-(trifluoromethyl)-1,6-dihydropyridin-3-yl)-1-oxo-3,4-dihydroisoquinolin-2(1H)-yl)methyl)nicotinonitrile;
4-ethoxy-6-((S)-1-(5-(6-fluoro-2-methylpyridin-3-yl)-7-(2-((2R, 3R)-3-hydroxy-2-methylpyrrolidin-1-yl)ethyl)-1-oxo-3,4-dihydroisoquinolin-2(1H)-yl)ethyl)nicotinonitrile;
4-ethoxy-6-((S)-1-(5-(6-fluoro-2-methylpyridin-3-yl)-7-(2-((2R,4S)-4-hydroxy-2-methylpyrrolidin-1-yl)ethyl)-1-oxo-3,4-dihydroisoquinolin-2(1H)-yl)ethyl)nicotinonitrile;
4-ethoxy-6-((S)-1-(5-(6-fluoro-2-methylpyridin-3-yl)-7-(2-((2S,4S)-4-hydroxy-2-methylpyrrolidin-1-yl)ethyl)-1-oxo-3,4-dihydroisoquinolin-2(1H)-yl)ethyl)nicotinonitrile;
(S)-4-ethoxy-6-(1-(5-(5-fluoro-2-(trifluoromethyl)pyridin-3-yl)-7-(2-((2-hydroxyethyl)(methyl)amino)ethyl)-1-oxo-3,4-dihydroisoquinolin-2(1H)-yl)ethyl)nicotinonitrile;
(S)-6-(1-(7-(2-(cyclopropyl(methyl)amino)ethyl)-5-(6-fluoro-2-methylpyridin-3-yl)-1-oxo-3,4-dihydroisoquinolin-2(1H)-yl)ethyl)-4-ethoxynicotinonitrile;
(S)-6-(1-(5-(5-amino-2-(trifluoromethyl)pyridin-3-yl)-7-(2-((2-hydroxyethyl)(methyl)amino)ethyl)-1-oxo-3,4-dihydroisoquinolin-2(1H)-yl)ethyl)-4-ethoxynicotinonitrile;
4-ethoxy-6-(1-(5-(6-fluoro-2-methylpyridin-3-yl)-7-(2-(methyl(oxetan-3-yl)amino)ethyl)-1-oxo-3,4-dihydroisoquinolin-2(1H)-yl)ethyl)nicotinonitrile;
(S)-6-(1-(5-(2,5-dimethylpyridin-4-yl)-7-(2-((2-methoxyethyl)(methyl)amino)ethyl)-1-oxo-3,4-dihydroisoquinolin-2(1H)-yl)ethyl)-4-ethoxynicotinonitrile;
6-(1-(5-(4-chloro-1-methyl-6-oxo-1,6-dihydropyridin-3-yl)-7-(2-((2-methoxyethyl)(methyl)amino)ethyl)-1-oxo-3,4-dihydroisoquinolin-2(1H)-yl)ethyl)-4-ethoxynicotinonitrile;
(S)-4-ethoxy-6-(1-(5-(6-fluoro-2-methylpyridin-3-yl)-7-(2-((2-methoxyethyl)(methyl)amino)ethyl)-1-oxo-3,4-dihydroisoquinolin-2(1H)-yl)ethyl)nicotinonitrile;
4-ethoxy-6-((S)-1-(5-(6-fluoro-2-methylpyridin-3-yl)-7-(2-((R)-2-methylazetidin-1-yl)ethyl)-1-oxo-3,4-dihydroisoquinolin-2(1H)-yl)ethyl)nicotinonitrile;
4-ethoxy-6-(1-(5-(6-fluoro-2-methylpyridin-3-yl)-7-(2-((2-fluoroethyl)(methyl)amino)ethyl)-1-oxo-3,4-dihydroisoquinolin-2(1H)-yl)ethyl)nicotinonitrile;
6-(1-(5-(4-chloro-1-methyl-6-oxo-1,6-dihydropyridin-3-yl)-7-(2-((2-fluoroethyl)(methyl)amino)ethyl)-1-oxo-3,4-dihydroisoquinolin-2(1H)-yl)ethyl)-4-ethoxynicotinonitrile;
(S)-6-(1-(5-(4-chloro-1-methyl-6-oxo-1,6-dihydropyridin-3-yl)-7-(2-((2-hydroxyethyl)(methyl)amino)ethyl)-1-oxo-3,4-dihydroisoquinolin-2(1H)-yl)ethyl)-4-ethoxynicotinonitrile;
6-((1S)-1-(5-(4-chloro-1-methyl-6-oxo-1,6-dihydropyridin-3-yl)-7-(2-((2-hydroxypropyl)(methyl)amino)ethyl)-1-oxo-3,4-dihydroisoquinolin-2(1H)-yl)ethyl)-4-ethoxynicotinonitrile;
(S)-6-(1-(5-(4-chloro-1-methyl-6-oxo-1,6-dihydropyridin-3-yl)-7-(2-((2-methoxyethyl)(methyl)amino)ethyl)-1-oxo-3,4-dihydroisoquinolin-2(1H)-yl)ethyl)-4-ethoxynicotinonitrile;
6-(1-(5-(1,3-dimethyl-1H-pyrazol-4-yl)-7-(2-((2-fluoroethyl)(methyl)amino)ethyl)-1-oxo-3,4-dihydroisoquinolin-2(1H)-yl)ethyl)-4-ethoxynicotinonitrile;
4-ethoxy-6-((1 S)-1-(5-(6-fluoro-2-methylpyridin-3-yl)-7-(2-((2-hydroxypropyl)(methyl)amino)ethyl)-1-oxo-3,4-dihydroisoquinolin-2(1H)-yl)ethyl)nicotinonitrile;
4-ethoxy-6-((S)-1-(5-(6-fluoro-2-methylpyridin-3-yl)-7-(2-((S)-2-(hydroxymethyl)azetidin-1-yl)ethyl)-1-oxo-3,4-dihydroisoquinolin-2(1H)-yl)ethyl)nicotinonitrile;
4-ethoxy-6-((S)-1-(5-(6-fluoro-2-methylpyridin-3-yl)-7-(2-((2R,3R)-3-methoxy-2-methylazetidin-1-yl)ethyl)-1-oxo-3,4-dihydroisoquinolin-2(1H)-yl)ethyl)nicotinonitrile;
(R)-4-ethoxy-6-((5-(6-fluoro-2-methylpyridin-3-yl)-7-(2-(2-methylazetidin-1-yl)ethyl)-1-oxo-3,4-dihydroisoquinolin-2(1H)-yl)methyl)nicotinonitrile;
6-((1S)-1-(5-(2,5-dimethylpyridin-3-yl)-7-(2-((2-hydroxypropyl)(methyl)amino)ethyl)-1-oxo-3,4-dihydroisoquinolin-2(1H)-yl)ethyl)-4-ethoxynicotinonitrile;
(S)-4-ethoxy-6-(1-(5-(6-fluoro-2-methylpyridin-3-yl)-7-(2-((2-fluoroethyl)(methyl)amino)ethyl)-1-oxo-3,4-dihydroisoquinolin-2(1H)-yl)ethyl)nicotinonitrile;
6-(1-(5-(2,5-dimethylpyridin-4-yl)-7-(2-((2-fluoroethyl)(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(3-fluoroazetidin-1-yl)ethyl)-5-(1-methyl-6-oxo-4-(trifluoromethyl)-1,6-dihydropyridin-3-yl)-1-oxo-3,4-dihydroisoquinolin-2(1H)-yl)ethyl)nicotinonitrile;
(S)-6-(1-(5-(2,5-dimethylpyridin-4-yl)-7-(2-((2-hydroxyethyl)(methyl)amino)ethyl)-1-oxo-3,4-dihydroisoquinolin-2(1H)-yl)ethyl)-4-ethoxynicotinonitrile;
(S)-6-(1-(5-(4-chloro-1-methyl-6-oxo-1,6-dihydropyridin-3-yl)-7-(2-((2-fluoroethyl)(methyl)amino)ethyl)-1-oxo-3,4-dihydroisoquinolin-2(1H)-yl)ethyl)-4-ethoxynicotinonitrile;
(S)-4-ethoxy-6-(1-(5-(6-fluoro-2-methylpyridin-3-yl)-7-(2-((2-hydroxyethyl)(methyl)amino)ethyl)-1-oxo-3,4-dihydroisoquinolin-2(1H)-yl)ethyl)nicotinonitrile;
(S)-4-ethoxy-6-(1-(5-(6-fluoro-2-methylpyridin-3-yl)-7-(2-(methyl(oxetan-3-yl)amino)ethyl)-1-oxo-3,4-dihydroisoquinolin-2(1H)-yl)ethyl)nicotinonitrile;
(S)-6-(1-(5-(2,5-dimethylpyridin-4-yl)-7-(2-((2-fluoroethyl)(methyl)amino)ethyl)-1-oxo-3,4-dihydroisoquinolin-2(1H)-yl)ethyl)-4-ethoxynicotinonitrile;
7-(2-(dimethylamino)ethyl)-2-(4-fluoro-3-methoxybenzyl)-5-(1-methyl-3-(trifluoromethyl)-1H-pyrazol-4-yl)-3,4-dihydroisoquinolin-1(2H)-one;
4-ethoxy-6-((1S)-1-(7-(2-(ethyl(methyl)amino)ethyl)-5-(1-hydroxy-2-methylpropyl)-1-oxo-3,4-dihydroisoquinolin-2(1H)-yl)ethyl)nicotinonitrile;
4-ethoxy-6-(1-(7-(2-(ethyl(methyl)amino)ethyl)-5-(1-hydroxy-2,2-dimethylpropyl)-1-oxo-3,4-dihydroisoquinolin-2(1H)-yl)ethyl)nicotinonitrile;
4-ethoxy-6-(1-(7-(2-(ethyl(methyl)amino)ethyl)-1-oxo-5-(3,3,3-trifluoro-1-hydroxy-2-methylpropyl)-3,4-dihydroisoquinolin-2(1H)-yl)ethyl)nicotinonitrile;
4-ethoxy-6-(1-(7-(2-(ethyl(methyl)amino)ethyl)-1-oxo-5-(2,2,2-trifluoro-1-hydroxyethyl)-3,4-dihydroisoquinolin-2(1H)-yl)ethyl)nicotinonitrile;
2-(1-(4-ethoxy-5-fluoropyridin-2-yl)ethyl)-7-(2-(ethyl(methyl)amino)ethyl)-5-(2,2,2-trifluoro-1-hydroxyethyl)-3,4-dihydroisoquinolin-1(2H)-one;
(R)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
(R)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
6-((5-(1,3-dimethyl-1H-pyrazol-4-yl)-7-((2-methyl-1H-imidazol-1-yl)methyl)-1-oxo-3,4-dihydroisoquinolin-2(1H)-yl)methyl)-4-ethoxynicotinonitrile;
(S)-6-(1-(5-(1,3-dimethyl-1H-1,2,4-triazol-5-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(3,5-dimethyl-1H-1,2,4-triazol-1-yl)-7-((2-methyl-1H-imidazol-1-yl)methyl)-1-oxo-3,4-dihydroisoquinolin-2(1H)-yl)ethyl)-4-ethoxynicotinonitrile;
4-ethoxy-6-((S)-1-(7-((2-methyl-1H-imidazol-1-yl)methyl)-5-((S)-1-methylpyrrolidin-2-yl)-1-oxo-3,4-dihydroisoquinolin-2(1H)-yl)ethyl)nicotinonitrile;
4-ethoxy-6-((S)-1-(7-((2-methyl-1H-imidazol-1-yl)methyl)-5-((R)-1-methylpyrrolidin-2-yl)-1-oxo-3,4-dihydroisoquinolin-2(1H)-yl)ethyl)nicotinonitrile;
(S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinamide;
(S)-4-ethoxy-6-(1-(7-((2-methyl-1H-imidazol-1-yl)methyl)-1-oxo-5-(6-oxo-4-(trifluoromethyl)-1,6-dihydropyridin-3-yl)-3,4-dihydroisoquinolin-2(1H)-yl)ethyl)nicotinonitrile;
(S)-6-(1-(5-(3-((dimethylamino)methyl)-1-methyl-1H-pyrazol-5-yl)-7-((2-methyl-1H-imidazol-1-yl)methyl)-1-oxo-3,4-dihydroisoquinolin-2(1H)-yl)ethyl)-4-ethoxynicotinonitrile;
(S)-4-ethoxy-6-(1-(5-(1-(2-hydroxyethyl)-3-methyl-1H-pyrazol-4-yl)-7-((2-methyl-1H-imidazol-1-yl)methyl)-1-oxo-3,4-dihydroisoquinolin-2(1H)-yl)ethyl)nicotinonitrile;
(S)-6-(1-(7-((2,4-dimethyl-1H-imidazol-1-yl)methyl)-5-(1-(2-hydroxyethyl)-3-methyl-1H-pyrazol-4-yl)-1-oxo-3,4-dihydroisoquinolin-2(1H)-yl)ethyl)-4-ethoxynicotinonitrile;
(S)-6-(1-(7-((2,4-dimethyl-1H-imidazol-1-yl)methyl)-5-(1,3-dimethyl-1H-pyrazol-4-yl)-1-oxo-3,4-dihydroisoquinolin-2(1H)-yl)ethyl)-4-ethoxynicotinonitrile;
(S)-6-(1-(7-((2,4-dimethyl-1H-imidazol-1-yl)methyl)-5-(1,4-dimethyl-6-oxo-1,6-dihydropyridin-3-yl)-1-oxo-3,4-dihydroisoquinolin-2(1H)-yl)ethyl)-4-ethoxynicotinonitrile;
(S)-6-(1-(5-(1,4-dimethyl-6-oxo-1,6-dihydropyridin-3-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(1,3-dimethyl-1H-pyrazol-4-yl)-7-((4-methyl-1H-imidazol-1-yl)methyl)-1-oxo-3,4-dihydroisoquinolin-2(1H)-yl)ethyl)-4-ethoxynicotinonitrile;
(S)-6-(1-(5-(1,4-dimethyl-1H-pyrazol-3-yl)-7-((2-methyl-1H-imidazol-1-yl)methyl)-1-oxo-3,4-dihydroisoquinolin-2(1H)-yl)ethyl)-4-ethoxynicotinonitrile;
(S)-2-(1-(4-ethoxy-5-fluoropyridin-2-yl)ethyl)-7-((2-methyl-1H-imidazol-1-yl)methyl)-5-(1-methyl-6-oxo-4-(trifluoromethyl)-1,6-dihydropyridin-3-yl)-3,4-dihydroisoquinolin-1(2H)-one;
(S)-4-ethoxy-6-(1-(7-((2-methyl-1H-imidazol-1-yl)methyl)-5-(1-methyl-1H-pyrazol-5-yl)-1-oxo-3,4-dihydroisoquinolin-2(1H)-yl)ethyl)nicotinonitrile;
(S)-6-(1-(5-(1,3-dimethyl-1H-pyrazol-5-yl)-7-((2-methyl-1H-imidazol-1-yl)methyl)-1-oxo-3,4-dihydroisoquinolin-2(1H)-yl)ethyl)-4-ethoxynicotinonitrile;
(S)-5-(1,4-dimethyl-6-oxo-1,6-dihydropyridin-3-yl)-2-(1-(4-ethoxy-5-fluoropyridin-2-yl)ethyl)-7-((2-methyl-1H-imidazol-1-yl)methyl)-3,4-dihydroisoquinolin-1(2H)-one;
(S)-6-(1-(5-(5-(difluoromethyl)-2-methylpyridin-3-yl)-7-((2-methyl-1H-imidazol-1-yl)methyl)-1-oxo-3,4-dihydroisoquinolin-2(1H)-yl)ethyl)-4-ethoxynicotinonitrile;
(S)-2-(1-(4-ethoxy-5-fluoropyridin-2-yl)ethyl)-5-(5-fluoro-1,4-dimethyl-6-oxo-1,6-dihydropyridin-3-yl)-7-((2-methyl-1H-imidazol-1-yl)methyl)-3,4-dihydroisoquinolin-1(2H)-one;
(R)-4-ethoxy-6-(1-(5-(3-methyl-1-(methylsulfonyl)-1H-pyrazol-4-yl)-7-((2-methyl-1H-imidazol-1-yl)methyl)-1-oxo-3,4-dihydroisoquinolin-2(1H)-yl)ethyl)nicotinonitrile;
(S)-6-(1-(5-(4-(difluoromethyl)-1-methyl-6-oxo-1,6-dihydropyridin-3-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(1-(difluoromethyl)-3-methyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
(S)-4-ethoxy-6-(1-(5-(5-fluoro-1,4-dimethyl-6-oxo-1,6-dihydropyridin-3-yl)-7-((2-methyl-1H-imidazol-1-yl)methyl)-1-oxo-3,4-dihydroisoquinolin-2(1H)-yl)ethyl)nicotinonitrile;
(S)-4-ethoxy-6-(1-(7-((2-methyl-1H-imidazol-1-yl)methyl)-5-(1-methyl-6-oxo-4-(trifluoromethyl)-1,6-dihydropyridin-3-yl)-1-oxo-3,4-dihydroisoquinolin-2(1H)-yl)ethyl)nicotinonitrile;
(S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(2,5-dimethyl-2H-1,2,3-triazol-4-yl)-7-((2-methyl-1H-imidazol-1-yl)methyl)-1-oxo-3,4-dihydroisoquinolin-2(1H)-yl)ethyl)-4-ethoxynicotinonitrile;
(S)-4-ethoxy-6-(1-(7-((2-methyl-1H-imidazol-1-yl)methyl)-5-(1-methyl-6-oxo-4-(trifluoromethyl)-1,6-dihydropyridazin-3-yl)-1-oxo-3,4-dihydroisoquinolin-2(1H)-yl)ethyl)nicotinonitrile;
(S)-6-(1-(5-(4-chloro-1-methyl-6-oxo-1,6-dihydropyridin-3-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(3-(difluoromethyl)-1-methyl-1H-1,2,4-triazol-5-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(5-(difluoromethyl)-3-methyl-1H-1,2,4-triazol-1-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-((dimethylamino)methyl)-7-((2-methyl-1H-imidazol-1-yl)methyl)-1-oxo-3,4-dihydroisoquinolin-2(1H)-yl)ethyl)-4-ethoxynicotinonitrile;
(S)-4-methoxy-6-(1-(7-((2-methyl-1H-imidazol-1-yl)methyl)-5-(1-methyl-6-oxo-4-(trifluoromethyl)-1,6-dihydropyridazin-3-yl)-1-oxo-3,4-dihydroisoquinolin-2(1H)-yl)ethyl)nicotinonitrile;
4-ethoxy-6-((7-((2-methyl-1H-imidazol-1-yl)methyl)-5-(1-methyl-6-oxo-4-(trifluoromethyl)-1,6-dihydropyridin-3-yl)-1-oxo-3,4-dihydroisoquinolin-2(1H)-yl)methyl)nicotinonitrile;
6-((5-(5-(difluoromethyl)-3-methyl-1H-1,2,4-triazol-1-yl)-7-((2-methyl-1H-imidazol-1-yl)methyl)-1-oxo-3,4-dihydroisoquinolin-2(1H)-yl)methyl)-4-ethoxynicotinonitrile;
6-((5-(4-(difluoromethyl)-1-methyl-6-oxo-1,6-dihydropyridin-3-yl)-7-((2-methyl-1H-imidazol-1-yl)methyl)-1-oxo-3,4-dihydroisoquinolin-2(1H)-yl)methyl)-4-ethoxynicotinonitrile;
6-((5-(1,3-dimethyl-1H-pyrazol-5-yl)-7-((2-methyl-1H-imidazol-1-yl)methyl)-1-oxo-3,4-dihydroisoquinolin-2(1H)-yl)methyl)-4-ethoxynicotinonitrile;
4-ethoxy-6-((5-(3-methyl-1-(methylsulfonyl)-1H-pyrazol-4-yl)-7-((2-methyl-1H-imidazol-1-yl)methyl)-1-oxo-3,4-dihydroisoquinolin-2(1H)-yl)methyl)nicotinonitrile;
(S)-4-ethoxy-6-(1-(7-((2-methyl-1H-imidazol-1-yl)methyl)-5-(3-methyl-1H-pyrazol-4-yl)-1-oxo-3,4-dihydroisoquinolin-2(1H)-yl)ethyl)nicotinonitrile;
(S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxypyridazine-3-carbonitrile;
4-ethoxy-6-(1-(7-(2-(ethyl(methyl)amino)ethyl)-1-oxo-5-(1,1,1-trifluoro-2-hydroxypropan-2-yl)-3,4-dihydroisoquinolin-2(1H)-yl)ethyl)nicotinonitrile, and
4-ethoxy-6-((7-(2-(ethyl(methyl)amino)ethyl)-1-oxo-5-(2,2,2-trifluoro-1-methoxyethyl)-3,4-dihydroisoquinolin-2(1H)-yl)methyl)nicotinonitrile.
   Embodiment 124. A compound selected from:
(S)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(R)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(S)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
(R)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
2-(1-(6-fluoro-5-methoxypyridin-3-yl)ethyl)-7-((2-(methylamino)-1H-imidazol-1-yl)methyl)-3,4-dihydroisoquinolin-1(2H)-one;
(S)-4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile;
(R)-4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile;
4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile;
(S)-6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(R)-6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-((7-(2-(ethyl(methyl)amino)ethyl)-5-(1-methyl-6-oxo-4-(trifluoromethyl)-1,6-dihydropyridin-3-yl)-1-oxo-3,4-dihydroisoquinolin-2(1H)-yl)methyl)nicotinonitrile;
(S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
(R)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile, and
6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile.
   Embodiment 125. A compound selected from:
(S)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(S)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
(S)-4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile;
(S)-6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-((7-(2-(ethyl(methyl)amino)ethyl)-5-(1-methyl-6-oxo-4-(trifluoromethyl)-1,6-dihydropyridin-3-yl)-1-oxo-3,4-dihydroisoquinolin-2(1H)-yl)methyl)nicotinonitrile, and
(S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile.

Depending on the choice of the starting materials hand procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the present invention. “Salts” include in particular “pharmaceutical acceptable salts”. The terms “pharmaceutically acceptable salt” or “pharmaceutically acceptable salts”, as used herein, refers to a salt or salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids. The organic acid or inorganic acids used to form pharmaceutically acceptable acid addition salts of compounds of the present invention include, but are not limited to, acetic acid, adipic acid, ascorbic acid, aspartic acid, benzoic acid, benzenesulfonic acid, carbonic acid, camphor sulfonic acid, capric acid, chlorotheophyllinate, citric acid, ethanedisulfonic acid, fumaric acid, D-glycero-D-gulo-Heptonic acid, galactaric aid, galactaric acid/mucic acid, gluceptic acid, glucoheptonoic acid, gluconic acid, glucuronic acid, glutamatic acid, glutaric acid, glycolic acid, hippuric acid, hydrobromic acid, hydrochloric acid, hydroiodic acid, isethionic acid, lactic acid, lactobionic acid, lauryl sulfuric acid, malic acid, maleic acid, malonic acid, mandelic acid, mesylic acid, methanesulfonic acid, mucic acid, naphthoic acid, 1-hydroxy-2-naphthoic acid, naphthalenesulfonic acid, 2-naphthalenesulfonic acid, nicotinic acid, nitric acid, octadecanoic acid, oleaic acid, oxalic acid, palmitic acid, pamoic acid, phosphoric acid, polygalacturonic acid, propionic acid, sebacic acid, stearic acid, succinic acid, sulfosalicylic acid, sulfuric acid, tartaric acid, p-toluenesulfonic acid, trifluoroacetic acid and triphenylacetic acid.

Salt forms of the compounds of the present invention can be converted into the free compounds by treatment with a suitable basic agent.

Pharmaceutically acceptable acid addition salts of compounds of the present invention include, but are not limited to, a acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlorotheophyllinate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete and xinafoate salt forms.

Pharmaceutically acceptable base addition salts can be formed with inorganic and organic bases. Organic bases used to form pharmaceutically acceptable base addition salts of compounds of the present invention include, but are not limited to, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine. Inorganic bases used to form pharmaceutically acceptable base addition salts of compounds of the present invention include, but are not limited to, sodium hydroxide, potassium hydroxide, ammonium hydroxide, ammonium salts and metals from columns I to XII of the periodic table. Pharmaceutically acceptable base addition salts of compounds of the present invention include, but are not limited to, sodium, potassium, ammonium, calcium, magnesium, iron, silver, zinc, and copper salts; particularly suitable salts include ammonium, potassium, sodium, calcium and magnesium salts.

In one embodiment, the present invention provides (S)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one in an acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlortheophyllonate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete or xinafoate salt form.

In one embodiment, the present invention provides (S)-2-(1-(6-fluoro-5-methoxypyridin-3-yl)ethyl)-7-((2-(methylamino)-1H-imidazol-1-yl)methyl)-3,4-dihydroisoquinolin-1(2H)-one in an acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlortheophyllonate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete or xinafoate salt form.

In one embodiment, the present invention provides (S)-4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile in an acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlortheophyllonate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete or xinafoate salt form.

In one embodiment, the present invention provides (S)-6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile in an acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlortheophyllonate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete or xinafoate salt form.

In one embodiment, the present invention provides 4-ethoxy-6-((7-(2-(ethyl(methyl)amino)ethyl)-5-(1-methyl-6-oxo-4-(trifluoromethyl)-1,6-dihydropyridin-3-yl)-1-oxo-3,4-dihydroisoquinolin-2(1H)-yl)methyl)nicotinonitrile in an acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlortheophyllonate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete or xinafoate salt form.

In one embodiment, the present invention provides (S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile in an acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlortheophyllonate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete or xinafoate salt form.

In one embodiment, the present invention provides (S)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one in an oxalate salt form.

In one embodiment, the present invention provides (S)-2-(1-(6-fluoro-5-methoxypyridin-3-yl)ethyl)-7-((2-(methylamino)-1H-imidazol-1-yl)methyl)-3,4-dihydroisoquinolin-1(2H)-one in an oxalate salt form.

In one embodiment, the present invention provides (S)-4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile in an oxalate salt form.

In one embodiment, the present invention provides (S)-6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile in an oxalate salt form.

In one embodiment, the present invention provides 4-ethoxy-6-((7-(2-(ethyl(methyl)amino)ethyl)-5-(1-methyl-6-oxo-4-(trifluoromethyl)-1,6-dihydropyridin-3-yl)-1-oxo-3,4-dihydroisoquinolin-2(1H)-yl)methyl)nicotinonitrile in an oxalate salt form.

In one embodiment, the present invention provides (S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile in an oxalate salt form.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the present invention include, for example, isotopes of hydrogen.

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the present invention include isotopes of hydrogen, carbon, nitrogen, oxygen, phosphorous, fluorine, and chlorine, such as ³H, ¹¹C, ¹³C, ¹⁴C, ¹⁵N, ¹⁸F ³¹P, ³²P, ³⁵S, ³⁶Cl, ¹²³I, ¹²⁴I, ¹²⁵I respectively. Accordingly it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagent& in place of the non-labeled reagent previously employed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(2)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof. ‘Substantially pure’ or ‘substantially free of other isomers’ as used herein means the product contains less than 5%, and preferably less than 2%, of other isomers relative to the amount of the preferred isomer, by weight.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

### Processes for Making Compounds of Invention

General procedures for preparing compounds of the present invention are described herein. In the reactions described, reactive functional groups, for example hydroxy, amino, imino or carboxy groups, where these are desired in the final product, may be protected to avoid their unwanted participation in the reactions. Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999.

Compounds of the present invention were made by processes described herein and as illustrated in the Examples. The combination of various building blocks and intermediates described herein can be applied to yield compounds of the invention. Non-limiting examples of synthetic schemes used to make compounds of the present invention are illustrated in Schemes 1 to 3.

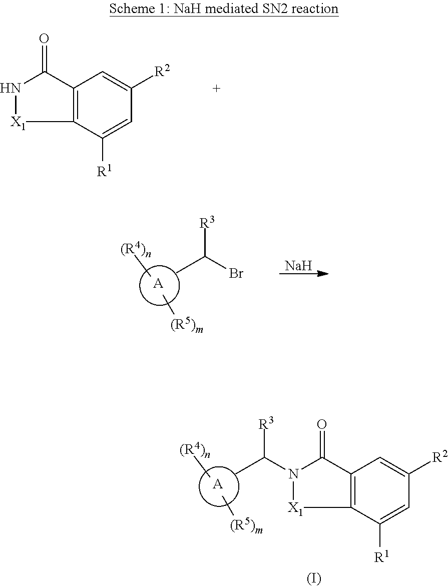

Compounds of formula (I) can be made using NaH mediated SN2 reactions as illustrated in Scheme 1, where X₁, R₁, R₂, R₃, R₄, R₅, m and n are as defined herein.

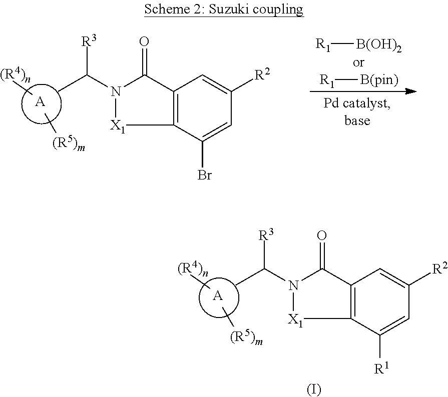

Compounds of formula (I) can be made using Suzuki coupling as illustrated in Scheme 2, where X₁, R₁, R₂, R₃, R₄, R₅, m and n are as defined herein.

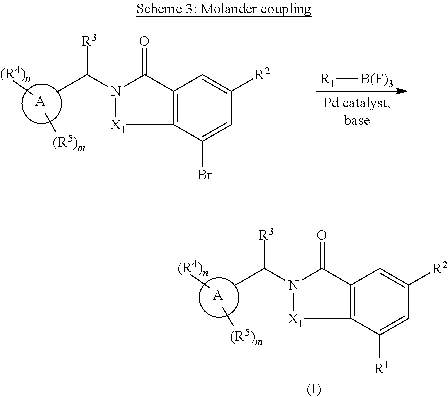

Compounds of formula (I) can be made using Molander coupling as illustrated in Scheme 2, where X₁, R₁, R₂, R₃, R₄, R₅, m and n are as defined herein.

### Administration and Pharmaceutical Compositions

In another aspect, the present invention provides a pharmaceutical composition comprising a compound of the present invention, or a pharmaceutically acceptable salt thereof, and one or more pharmaceutically acceptable carriers. The pharmaceutical composition can be formulated for particular routes of administration such as oral administration, parenteral administration, and rectal administration, and the like. In addition, the pharmaceutical compositions of the present invention can be made up in a solid form (including without limitation capsules, tablets, pills, granules, powders or suppositories), or in a liquid form (including without limitation solutions, suspensions or emulsions). Pills or tablets may be either film coated or enteric coated according to methods known in the art. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. Typically, the pharmaceutical compositions comprising compounds of the invention are tablets or gelatin capsules comprising a compound of Formula (I) as an active ingredient together with one or more of the following:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

The invention further provides pharmaceutical compositions optionally further comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

Selection of suitable capsules for encapsulation and of suitable pharmaceutically acceptable carriers for formulating the compound of Formula I to make oral dosage forms is within the ordinary level of skill. Tablets may be either film coated or enteric coated using methods known in the art.

Suitable compositions for oral administration include an therapeutically effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable carriers which are suitable for the manufacture of tablets, including the ones listed above. These pharmaceutically acceptable carriers are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil, to form a solution, emulsion or dispersion inside the soft capsule.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin. Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurized container, pump, spray, atomizer or nebulizer, with or without the use of a suitable propellant.

The present invention further provides anhydrous pharmaceutical compositions and dosage forms comprising the compounds of the present invention as active ingredients, where it is desirable to minimize exposure of the compound to water prior to administration. Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

The compounds of formula I, and subformulae thereof, in free form or in salt form, exhibit valuable pharmacological properties, e.g. they modulate or inhibit activity of WDR5, as indicated by test data provided in the following sections, and are therefore indicated for therapy as described herein, or for use as research chemicals, e.g. as tool compounds to further the understanding of the effects of WDR5 inhibition or inhibition of an associated biochemical pathway.

Compounds of the present invention may be useful in the treatment of a disease characterized by excessive or undesired levels of activity of WDR5. Accordingly, compounds of the present invention may be useful in the treatment of cancer, such as solid tumors, adenoma, bladder cancer, brain cancer, breast cancer, cervical cancer, colorectal cancer, colon cancer, epidermal carcinoma, follicular carcinoma, genitourinary cancers, glioblastoma, head and neck cancers, Hodgkin's disease, non-Hodgkin's lymphoma, hepatoma, kidney cancer, lung cancers such as small cell or non-small cell lung cancer, leukemias such as acute lymphoblastic leukemia (ALL), acute myelogenous leukemia (AML) and chronic myelogenous leukemia (CML), multiple myeloma, lymphoid disorders, skin cancers including melanoma, neuroblastoma, ovarian cancer, pancreatic cancer, prostate cancer, rectal cancer, sarcoma, testicular cancer, and thyroid cancer.

Compounds of the present invention may be useful in the treatment of leukemia, such as ALL, AML or CML. In certain embodiments, compounds of the present invention may be useful in the treatment of leukemia, such as ALL and AML.

Compounds of the present invention may be useful in the treatment pancreatic cancer, MLLwt AML, neuroblastoma, breast cancer, bladder cancer, colorectal cancer, pancreatic cancer and thyroid cancer.

Compounds of the present invention may be useful in the treatment of cancers due to dysregulated Myc.

Thus, as a further aspect, the present invention provides the use of a compound of the present invention, or pharmaceutically acceptable salt thereof, in therapy. In an embodiment, the therapy is selected from a disease which may be treated by inhibition of WDR5. In another embodiment, the disease is cancer, including but not limited to those listed above. In an embodiment the disease is a cancer selected from the afore-mentioned lists.

In a further aspect, the present invention provides a compound of the present invention, or or pharmaceutically acceptable salt thereof, for use in therapy. In an embodiment, the therapy is selected from a disease which may be treated by inhibition of WDR5. In another embodiment, the disease is cancer, including but not limited to those listed above. In an embodiment the disease is a cancer selected from the afore-mentioned lists.

Thus, as a further aspect, the present invention provides the use of a compound of formula (I), or any of the embodiments within the scope of Formula (I) as described herein, for the manufacture of a medicament. In an embodiment, the medicament is for the treatment of a disease which may be treated by inhibition of WDR5. In another embodiment, the compounds of the invention are useful to treat cancers, including but not limited to those listed above. In an embodiment the disease is a cancer selected from the afore-mentioned lists.

In another aspect, the invention provides a method of treating a disease which is treated by the inhibition of WDR5, where the method comprises the administration of a therapeutically effective amount of a compound of the present invention, or any of the embodiments within the scope of Formula (I) as described herein. In an embodiment, the disease is cancer, including but not limited to those listed above. In an embodiment the disease is a cancer selected from the afore-mentioned lists.

In another aspect, the invention provides a method of treating a disease which is treated by the inhibition of WDR5, where the method comprises the administration of a compound of the present invention, or any of the embodiments within the scope of Formula (I) as described herein. In an embodiment, the disease is cancer, including but not limited to those listed above. In an embodiment the disease is a cancer selected from the afore-mentioned lists.

The method typically comprises administering a therapeutically effective amount of a compound as described herein or a pharmaceutical composition comprising such compound to a subject in need of such treatment. The compound may be administered by any suitable method such as those described herein, and the administration may be repeated at intervals selected by a treating physician. The invention thus provides a compound of Formula (I), or any subforumulae thereof or pharmaceutically acceptable salt thereof, as described herein for use to treat a condition mediated by or associated with excessive or undesired levels of WDR5 activity, including those mentioned above.

In another aspect, the compounds of the present invention are used in combination with one or more additional therapeutic agents. Suitable additional therapeutic agents include other anticancer agents, analgesics, anti-inflammatory agents, and the like.

Accordingly, the invention provides the use of a compound of formula (I) for treating a disease or condition mediated by WDR5, wherein the medicament is prepared for administration with another therapeutic agent. The invention also provides the use of another therapeutic agent for treating a disease or condition, wherein the therapeutic agent is administered with a compound of formula (I).

The invention also provides a compound of formula (I) for use in a method of treating a disease or condition mediated by WDR5, wherein the compound of formula (I) is prepared for administration with another therapeutic agent. The invention also provides another therapeutic agent for use in a method of treating a disease or condition mediated by WDR5, wherein the other therapeutic agent is prepared for administration with a compound of formula (I). The invention also provides a compound of formula (I) for use in a method of treating a disease or condition mediated by WDR5, wherein the compound of formula (I) is administered with another therapeutic agent. The invention also provides another therapeutic agent for use in a method of treating a disease or condition mediated by WDR5, wherein the other therapeutic agent is administered with a compound of formula (I).

Accordingly, suitable additional therapeutic agents for use with the compounds of the invention are typically selected based on the condition for treatment. For example, in the treatment of melanoma, the additional therapeutic agent may be selected from Aldesleukin, Dabrafenib, Dacarbazine, DTIC-Dome (Dacarbazine), Intron A (Recombinant Interferon Alfa-2b), Ipilimumab, Mekinist (Trametinib), Peginterferon Alfa-2b, PEG-Intron (Peginterferon Alfa-2b), Proleukin (Aldesleukin), Recombinant Interferon Alfa-2b, Sylatron (Peginterferon Alfa-2b), Tafinlar (Dabrafenib), Trametinib, Vemurafenib, Yervoy (Ipilimumab), and Zelboraf (Vemurafenib). For the treatment of ovarian cancer, the additional therapeutic agent may be selected from Adriamycin PFS (Doxorubicin Hydrochloride), Adriamycin RDF (Doxorubicin Hydrochloride), Carboplatin, Clafen (Cyclophosphamide), Cisplatin, Cyclophosphamide, Cytoxan (Cyclophosphamide), Doxorubicin Hydrochloride, Dox-SL (Doxorubicin Hydrochloride Liposome), DOXIL (Doxorubicin Hydrochloride Liposome), Doxorubicin Hydrochloride Liposome, Evacet (Doxorubicin Hydrochloride Liposome), Gemcitabine Hydrochloride, Gemzar (Gemcitabine Hydrochloride), Hycamtin (Topotecan Hydrochloride), LipoDox (Doxorubicin Hydrochloride Liposome), Neosar (Cyclophosphamide), Paclitaxel, Paraplat (Carboplatin), Paraplatin (Carboplatin), Platinol (Cisplatin), Platinol-AQ (Cisplatin), Taxol (Paclitaxel), and Topotecan Hydrochloride. For the treatment of thyroid cancer, the additional therapeutic agent may be selected from Adriamycin PFS (Doxorubicin Hydrochloride), Adriamycin RDF (Doxorubicin Hydrochloride), Cabozantinib-S-Malate, Caprelsa (Vandetanib), Cometriq (Cabozantinib-S-Malate), Doxorubicin Hydrochloride, and Vandetanib. For the treatment of colon cancer, the co-therapeutic may be selected from Adrucil (Fluorouracil), Avastin (Bevacizumab), Bevacizumab, Camptosar (Irinotecan Hydrochloride), Capecitabine, Cetuximab, Efudex (Fluorouracil), Eloxatin (Oxaliplatin), Erbitux (Cetuximab) Fluoroplex (Fluorouracil), Fluorouracil, Irinotecan Hydrochloride, Leucovorin Calcium, Oxaliplatin, Panitumumab, Regorafenib, Stivarga (Regorafenib), Vectibix (Panitumumab), Wellcovorin (Leucovorin Calcium), Xeloda (Capecitabine), Zaltrap (Ziv-Aflibercept), and Ziv-Aflibercept. For the treatment of lung cancer, the co-therapeutic may be selected from Abitrexate (Methotrexate), Abraxane (Paclitaxel Albumin-stabilized Nanoparticle Formulation), Afatinib Dimaleate, Alimta (Pemetrexed Disodium), Avastin (Bevacizumab), Bevacizumab, Carboplatin, Cisplatin, Crizotinib, Erlotinib Hydrochloride, Folex (Methotrexate), Folex PFS (Methotrexate), Gefitinib, Gilotrif (Afatinib Dimaleate), Gemcitabine Hydrochloride, Gemzar (Gemcitabine Hydrochlorde), Iressa (Gefitinib), Methotrexate, Methotrexate LPF (Methotrexate), Mexate (Methotrexate), Mexate-AQ (Methotrexate), Paclitaxel, Paclitaxel Albumin-stabilized Nanoparticle Formulation, Paraplat (Carboplatin), Paraplatin (Carboplatin), Pemetrexed Disodium, Platinol (Cisplatin), Platinol-AQ (Cisplatin), Tarceva (Erlotinib Hydrochloride), Taxol (Paclitaxel), and, Xalkori (Crizotinib). For the treatment of pancreatic cancer, the additional therapeutic agent can be selected from Adrucil (Fluorouracil), Efudex (Fluorouracil), Erlotinib Hydrochloride, Fluoroplex (Fluorouracil), Fluorouracil, Gemcitabine Hydrochloride, Gemzar (Gemcitabine Hydrochloride), Mitomycin C, Mitozytrex (Mitomycin C), Mutamycin (Mitomycin) and Tarceva (Erlotinib Hydrochloride. For the treatment of cervical cancer, the additional therapeutic agent may be selected from Blenoxane (Bleomycin), Bleomycin, Cisplatin, Hycamtin (Topotecan Hydrochloride), Platinol (Cisplatin), Platinol-AQ (Cisplatin), and Topotecan Hydrochloride. For the treatment of head and neck cancer, the additional therapeutic agent may be selected from Abitrexate (Methotrexate), Adrucil (Fluorouracil), Blenoxane (Bleomycin), Bleomycin, Cetuximab, Cisplatin, Docetaxel, Efudex (Fluorouracil), Erbitux (Cetuximab), Fluoroplex (Fluorouracil), Fluorouracil, Folex (Methotrexate), Folex PFS (Methotrexate), Methotrexate, Methotrexate LPF (Methotrexate), Mexate (Methotrexate), Mexate-AQ (Methotrexate), Platinol (Cisplatin), Platinol-AQ (Cisplatin), and Taxotere (Docetaxel). For the treatment of leukemia, including CMML, the additional therapeutic agent can be selected from Bosulif (Bosutinib), Bosutinib, Clafen (Cyclophosphamide), Cyclophosphamide, Cytarabine, Cytosar-U (Cytarabine), Cytoxan (Cyclophosphamide), Dasatinib, Gleevec (Imatinib Mesylate), Iclusig (Ponatinib Hydrochloride) Imatinib Mesylate, Neosar (Cyclophosphamide), Nilotinib, Omacetaxine Mepesuccinate, Ponatinib Hydrochloride, Sprycel (Dasatinib), Synribo (Omacetaxine Mepesuccinate), Tarabine PFS (Cytarabine), and Tasigna (Nilotinib).

The invention also provides the use of a compound of formula (I) for treating a disease or condition mediated by WDR5, wherein the patient has previously (e.g. within 24 hours) been treated with another therapeutic agent. The invention also provides the use of another therapeutic agent for treating a disease or condition mediated by WDR5, wherein the patient has previously (e.g. within 24 hours) been treated with a compound of formula (I). Specific individual combinations which may provide particular treatment benefits include a compound of the invention with at least one compound selected from inhibitors of BRAF, MEK, CDK4/6, SHP-2, HDAC, EGFR, MET, mTOR, PI3K, and AKT. Examples of these inhibitors include vemurafinib, debrafinib, LGX818, trametinib, MEK162, LEE011, PD-0332991, panobinostat, verinostat, romidepsin, cetuximab, gefitinib, erlotinib, lapatinib, panitumumab, vandetanib, INC280, everolimus, simolimus, BMK120, BYL719, and CLR457.

The compound of the present invention may be administered either simultaneously with, or before or after, one or more additional therapeutic agents, also referred to herein as co-agent(s). The compound of the present invention may be administered separately, by the same or different route of administration, or together in the same pharmaceutical composition as the co-agent(s).

In one embodiment, the invention provides a pharmaceutical composition comprising a compound of formula (I) and another therapeutic co-agent(s). Optionally, the pharmaceutical composition may comprise a pharmaceutically acceptable carrier, as described above.

In one embodiment, the invention provides a product comprising a compound of formula (I) and at least one other therapeutic co-agent as a combined preparation for simultaneous, separate or sequential use in therapy. In one embodiment, the therapy is the treatment of a disease or condition mediated by WDR5, such as cancer. Products provided as a combined preparation include a composition comprising the compound of formula (I) and the other therapeutic co-agent(s) together in the same pharmaceutical composition, or the compound of formula (I) and the other therapeutic co-agent(s) in separate form, e.g. in the form of a kit.

In one embodiment, the invention provides a kit comprising two or more separate pharmaceutical compositions, at least one of which contains a compound of formula (I). In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

The invention also provides the use of a compound of formula (I) for treating a disease or condition mediated by WDR5, wherein the patient is one treated previously or subsequently (e.g. within 24 hours) with another therapeutic agent. The invention also provides the use of a an additional therapeutic agent for treating a disease or condition mediated by WDR5, wherein the patient has previously (e.g. within 24 hours) been treated with a compound of formula (I).

In the combination therapies of the invention, the compound of the invention and the other therapeutic co-agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

Certain aspects and examples of the pharmaceutical compositions, combinations, methods and uses of the present invention are provided in the following listing of additional, enumerated embodiments. It will be recognized that features specified in each embodiment may be combined with other specified features to provide further embodiments of the present invention.

Embodiment 126. A pharmaceutical composition comprising a compound according to any one of Embodiments 1-125, or a pharmaceutically acceptable salt thereof, and one or more pharmaceutically acceptable carriers.
Embodiment 127. The pharmaceutical composition of Embodiment 126, further comprising a therapeutic co-agent.
Embodiment 128. The pharmaceutical composition of Embodiment 127, wherein the therapeutic co-agent is selected from anticancer compounds, analgesics, and anti-inflammatory compounds.
Embodiment 129. A method to treat cancer, comprising administering to a subject in need of such treatment a therapeutically effective amount of a compound according to any of Embodiments 1-125 or a pharmaceutical composition of any of Embodiments 126 to 128.
Embodiment 130. A method to treat cancer, comprising administering to a subject in need of such treatment a compound according to any of Embodiments 1-125 or a pharmaceutical composition of any of Embodiments 126 to 128.
Embodiment 131. The method of Embodiment 129 or Embodiment 130, wherein the cancer is selected from adenoma, bladder cancer, brain cancer, breast cancer, cervical cancer, colorectal cancer, colon cancer, epidermal carcinoma, follicular carcinoma, genitourinary cancers, glioblastoma, head and neck cancers, Hodgkin's disease, non-Hodgkin's lymphoma, hepatoma, head and neck cancers, kidney cancer, lung cancers such as small cell or non-small cell lung cancer, leukemias such as acute lymphoblastic leukemia (ALL), acute myelogenous leukemia (AML) or chronic myelogenous leukemia (CML), multiple myeloma, lymphoid disorders, skin cancers including melanoma, neuroblastoma, ovarian cancer, pancreatic cancer, prostate cancer, rectal cancer, sarcoma, testicular cancer, and thyroid cancer.
Embodiment 132. A compound according to any one of Embodiments 1-125 for use as a medicament.
Embodiment 133. Use of a compound according to any one of Embodiments 1 to 125 or a pharmaceutically acceptable salt thereof in the manufacture of a medicament for the treatment of cancer; or use of a compound according to any one of Embodiments 1 to 125 or a pharmaceutically acceptable salt thereof in therapy, wherein the therapy is the treatment of cancer.
Embodiment 134. The use of Embodiment 133, wherein the cancer is selected from adenoma, bladder cancer, brain cancer, breast cancer, cervical cancer, colorectal cancer, colon cancer, epidermal carcinoma, follicular carcinoma, genitourinary cancers, glioblastoma, head and neck cancers, Hodgkin's disease, non-Hodgkin's lymphoma, hepatoma, head and neck cancers, kidney cancer, lung cancers such as small cell or non-small cell lung cancer, leukemias such as acute lymphoblastic leukemia (ALL), acute myelogenous leukemia (AML) or chronic myelogenous leukemia (CML), multiple myeloma, lymphoid disorders, skin cancers including melanoma, neuroblastoma, ovarian cancer, pancreatic cancer, prostate cancer, rectal cancer, sarcoma, testicular cancer, and thyroid cancer.

### EXAMPLES

The compounds of the present invention can be produced as shown in the following examples. The following examples are intended to illustrate the invention and are not to be construed as being limitations thereon. Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesize the compounds of the present invention are either commercially available or can be produced by organic synthesis methods known to one of ordinary skill in the art or can be produced by the organic synthesis methods as described herein.

For illustrative purposes, the general reaction schemes depicted herein provide potential routes for synthesizing the compounds of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### Abbreviations

Abbreviations used are those conventional in the art unless otherwise indicated or defined in the following list:

| | |
|---|---|
| Ac₂O | Acetic anhydride |
| AcOH or OHAc | Acetic acid |
| BINAP | racemic 2,2′- |
| | bis(diphenylphosphino)- |
| | 1,1′-binaphthyl |
| BnBr | Benzyl bromide |
| Boc | tertiary butoxycarbonyl |
| br | broad |
| B(OPr)₃ | Triisopropyl borate |
| BSA | bovine serum albumin |
| d | doublet |
| dd | doublet of doublets |
| DAST | Diethylaminosulfur |
| | trifluoride |
| DCM | dichloromethane |
| DIAD | Diisopropyl |
| | azodicarboxylate |
| DIEA | diethylisopropylamine |
| DMAP | 4-Dimethylaminopyridine |
| DMF | N,N-dimethylformamide |
| DMSO | dimethylsulfoxide |
| DPPF | 1,1′- |
| | Bis(diphenylphosphino)ferrocene |
| DPPP | 1,3- |
| | Bis(diphenylphosphino)propane |
| EA | Ethyl acetate |
| EDCI | N-Ethyl-N′-(3- |
| | dimethylaminopropyl)carbodiimide |
| | hydrochloride |
| ESI | electrospray ionization |
| EtOAc | ethyl acetate |
| EtONa | Sodium ethoxide |
| h., hrs. | hour, hours |
| HPLC | high pressure liquid |
| | chromatography |
| IPA | Isopropyl alcohol |
| LDA | Lithium diisopropylamide |
| M-CPBA | meta-Chloroperoxybenzoic |
| | acid |
| MeOH | methanol |
| MeCN | acetonitrile |
| MS | mass spectrometry |
| MTBE | Methyl tert-butyl ether |
| MW | microwave |
| m | multiplet |
| min., mins. | minute, minutes |
| mL | milliliter(s) |
| m/z | mass to charge ratio |
| NBS | N-Bromosuccinimide |
| NIS | N-Iodosuccinimide |
| NMR | nuclear magnetic |
| | resonance |
| ppm | parts per million |
| rac | racemic |
| rt or RT | room temperature |
| s | singlet |
| t | triplet |
| SEMCI | 2- |
| | (Trimethylsilyl)ethoxymethyl |
| | chloride |
| TBAF | Tetra-n-butylammonium |
| | fluoride |
| TBHP | tert-Butyl hydroperoxide |
| TEA | Triethyl amine |
| TFA | trifluoroacetic acid |
| TfOH | trifluoromethanesulfonic |
| | acid |
| THF | tetrahydrofuran |
| TLC | Thin-layer |
| | chromatography |
| Xphos | 2-Dicyclohexylphosphino- |
| | 2′,4′,6′- |
| | triisopropylbiphenyl |

### Analytical Instruments and Methods

High performance liquid chromatography (HPLC) was performed using an Agilent 1260 HPLC System (Santa Clara, Calif.). The analytical column was reversed phase Phenomenex Kinetex C18-2.6 μm, 4.6×50 mm. A gradient elution was used (flow rate 2.0 mL/min), starting with 5% methanol/95% water and progressing to 95% methanol/5% water over a period of 10 minutes. All solvents contained 0.1% formic acid (FA). Compounds were detected by ultraviolet light (UV) absorption at 214, 254 and 300 nm. HPLC solvents were purchased from Sigma Aldrich (St. Louis, Mo.).

Mass spectrometric analysis was performed on an Agilent System (Agilent 1260 HPLC and an Agilent 6130 mass spectrometer detector; Column: Phenomenex Kinetex 2.6 um C18, column size 4.6×50 mm; column temperature 40° C.; gradient: 5 95% methanol in water with 0.1% FA over a 2 min period; flow rate 2.0 mL/min (or Polar gradient 5-50% over 2.0 min, or Non-Polar gradient 50-95% over 2.0 min); Mass Spectrometer molecular weight scan range 100 1000; or 100-1500; capillary voltage 4000 V. All masses were reported as those of the protonated parent ions, unless otherwise indicated.

Nuclear magnetic resonance (NMR) analysis was performed using a Bruker 400 MHz NMR. The spectral reference was either TMS or the known chemical shift of the solvent.

### Chiral Preparative HPLC Methods Employed in Purification of Examples

SFC chiral screening was carried out on a Thar Insturments Investigator system. The Thar Investigator system consists of:
ALIAS autosampler
Thar Fluid Delivery Module (0 to 10 mL/min)
Thar SFC 10 position column oven
Waters 2998 PDA
Thar Automated Back Pressure Regulator

All of the Thar components are part of the SuperPure Discovery Series line. The system flowed at 3.0 mL/min and kept at 38° C. The system back pressure was set to 100 bar. Each sample was screened through a battery of ten 5 μm columns:
5 μm 4.6×150 mm ChiralPak AD
5 μm 4.6×150 mm ChiralCel OD
5 μm 4.6×150 mm ChiralCel OJ
5 μm 4.6×150 mm ChiralPak AS
5 μm 4.6×250 mm ChiralPak AY
5 μm 4.6×250 mm ChiralCel OZ
5 μm 4.6×150 mm ChiralPak IC
5 μm 4.6×150 mm ChiralPak IG
5 μm 4.6×250 mm Regis Whelk-O1
5 μm 4.6×250 mm ChromegaChiral CC4

The system ran a gradient from 5% co-solvent to 50% co-solvent in 9 minutes followed by a 10 minutes hold at 50% co-solvent, a switch back to 5% co-solvent and a 0.5 minute hold at initial condition. In between each gradient there was a 4 minute equilibration method that flows 5% co-solvent through the next column to be screened. The typical solvents screened were, MeOH, EtOH, IPA, MeOH+0.5% NH₃, EtOH+0.5% NH₃, IPA+0.1% NH₃. Once separation was detected using one of the gradient methods, an isocratic method can be developed, and if necessary, scaled up for separation on the Thar Prep 80 system.

### Synthesis of Intermediates

### Intermediate A1: 5-bromo-7-((2-(methylamino)-1H-imidazol-1-yl)methyl)-3,4-dihydroisoquinolin-1(2H)-one (A1)

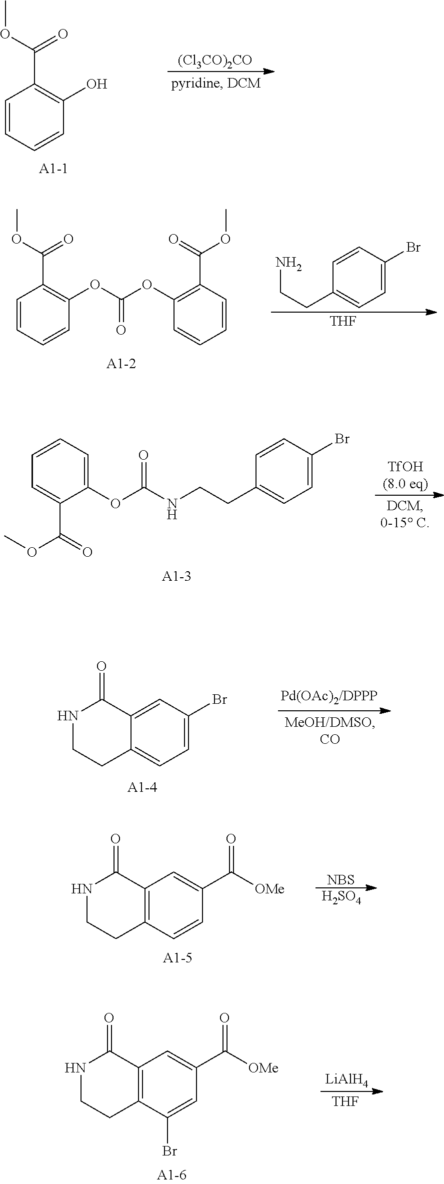

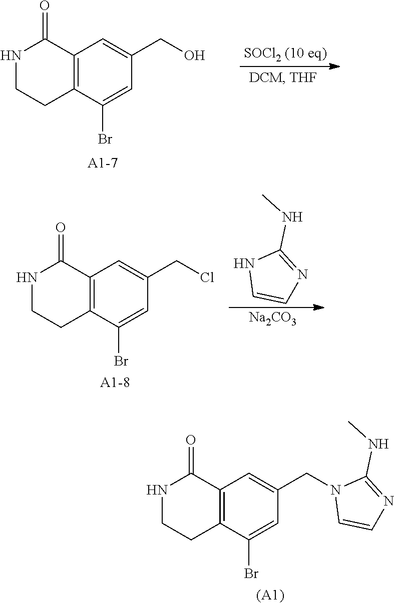

Step 1: To a solution of methyl 2-hydroxybenzoate (A1-1) (2.5 kg, 16.4 mol, 1.0 eq.) and pyridine (7.77 kg, 98.4 mol, 6.0 eq.) in dichloromethane (25 L) was added a solution of triphosgene (1.2 kg, 4 mol, 0.25 eq.) in dichloromethane (5 L) dropwise during a period of 2 hrs at 0-25° C. The mixture was poured into a mixture of sat.NH₄Cl (5 L) and water (10 L) carefully. The organic layer was separated and the aqueous layer was extracted with dichloromethane (2 L twice). The organic layer were combined and concentrated under reduced pressure. To the residue was added petroleum ether (20 L) and stirred for 30 min, then filtered. The solid was dried in vacuum to give dimethyl 2,2′-(carbonylbis(oxy))dibenzoate (A1-2) (2.3 kg, 85% yield) as a white solid. ¹H NMR (400 MHz, CDCl₃) δ 8.05 (d, J=7.9 Hz, 1H), 7.66-7.58 (m, 1H), 7.43-7.34 (m, 2H), 3.96 (s, 3H).

Step 2: To a solution of compound A1-2 (2.5 kg, 7.57 mol, 1.0 eq.) in THF (25 L), 2-(4-bromophenyl)ethan-1-amine (1.5 kg, 7.57 mol, 1.0 eq.) in tetrahydrofuran (5 L) was added dropwise over a 2 hour period. The mixture was then stirred at 15° C. for 18 hrs and then concentrated. To the residue was added petroleum ether (8 L). The mixture was stirred for 20 mins and then filtered. The solid was dried in vacuum to give methyl 2-(((4-bromophenethyl)carbamoyl)oxy)benzoate (A1-3) (2.6 kg, 91% yield) as a white solid. ¹H NMR (400 MHz, CDCl₃) δ 7.99 (d, J=7.8 Hz, 1H), 7.56 (t, J=7.7 Hz, 1H), 7.47 (d, J=8.3 Hz, 2H), 7.35-7.27 (m, 1H), 7.21-7.14 (m, 3H), 5.24 (br s, 1H), 3.88 (s, 3H), 3.53 (q, J=6.7 Hz, 2H), 2.89 (br t, J=7.0 Hz, 2H).

Step 3: To a solution of compound A1-3 (1.6 kg, 4.23 mol, 1.0 eq.) in dichloromethane (25 L) was added TfOH (5 kg, 33.8 mol, 8.0 eq.) at 0° C. dropwise. The mixture was stirred at 15° C. for 5 hrs. The mixture was poured into ice water (10 L) carefully. The aqueous layer was extracted with dichloromethane (10 L twice). The organic layers were combined and concentrated. To the residue was added MTBE (2.5 L). The suspension was stirred for 1 hr and filtered. The solid was dried in vacuum and the two parallel batches was combined to give 7-bromo-3,4-dihydroisoquinolin-1(2H)-one (A1-4) (1.55 kg (from 3.2 kg of compound 3), yield 80%) as a white solid. ¹H NMR (400 MHz, CDCl₃) δ 8.21 (d, J=2.1 Hz, 1H), 7.58 (dd, J=2.1, 8.0 Hz, 1H), 7.18-6.99 (m, 2H), 3.59 (dt, J=2.9, 6.7 Hz, 2H), 2.97 (t, J=6.7 Hz, 2H).

Step 4: A solution of compound A1-4 (400 g, 1.77 mol, 1.0 eq.), Pd(OAc)₂ (39.7 g, 0.177 mol, 0.1 eq.), DPPP (72 g, 0.177 mol, 0.1 eq.), Et₃N (268 g, 2.65 mol, 1.5 eq.) in dimethylsulfoxide (2.5 L) and methanol (3 L) was stirred at 80° C. under CO (50 psi) atmosphere for 18 hrs. The mixture was poured into water (13 L). The aqueous layer was extracted with ethyl acetate (6 L, three times), washed with saturated salt water, and then dried over anhydrous sodium sulfate. The organic layers were combined and concentrated. To the residue was added acetone (2.5 L) and the mixture was stirred for 1 h. The solid was obtained by filtration and dried in vacuum to give methyl 1-oxo-1,2,3,4-tetrahydroisoquinoline-7-carboxylate (A1-5) (750 g, yield 68.9% from 3 batches using a total of 1.2 kg of compound A1-4) as a grey solid. ¹HNMR (400 MHz, CDCl₃) δ 8.73 (d, J=1.5 Hz, 1H), 8.13 (dd, J=1.8, 7.9 Hz, 1H), 7.33 (d, J=7.9 Hz, 1H), 3.94 (s, 3H), 3.62 (dt, J=2.8, 6.6 Hz, 2H), 3.07 (t, J=6.5 Hz, 2H).

Step 5: To three individual solutions of compound A1-5 (283 g, 1.38 mol, 1.0 eq) in con.H₂SO₄ (2.5 L) was added NBS (368 g, 2.0 mmol, 1.5 eq). The mixtures were then stirred at 40-45° C. for 1 hour. The 3 parallel reactions were poured into ice water (60 L) slowly and then filtered. The wet cake was washed with water and then slurried in ethanol (5 L) for 1 h. The mixture was filtered and the cake was washed with ethanol. The solid was dried in vacuum to give methyl 5-bromo-1-oxo-1,2,3,4-tetrahydroisoquinoline-7-carboxylate (A1-6) (1.05 kg, 89% yield) as a yellow solid. ¹H NMR (400 MHz, DMSO-d⁶) δ ppm 8.41 (s, 1H), 8.25 (br s, 1H), 8.22 (s, 1H), 3.89 (s, 3H), 3.44 (dt, J=2.8, 6.6 Hz, 2H), 3.04 (t, J=6.7 Hz, 2H).

Step 6: To a solution of compound A1-6 (550 g, 1.93 mol, 1.0 eq.) in tetrahydrofuran (22 L) was added LiAlH₄ (147 g, 3.86 mol, 2.0 eq.) in portions at 0-10° C. The mixture was stirred at 10-25° C. for 2 hrs. TLC (100% ethyl acetate) showed most of compound A1-6 was consumed. The reaction was quenched with water (147 mL) followed by aq. 15% NaOH (147 mL) and water (441 mL). The mixture was stirred for 0.5 hr then filtered. The cake was slurried in dichloromethane/methanol (5 L/5 L) and filtered. The organic layers were combined and concentrated. The solid was slurried in ethanol/water (5.1 L/1.7 L) at 15° C. for 2 hrs and filtered again to remove inorganic salt. The obtained solid was dried in vacuum to give 5-bromo-7-(hydroxymethyl)-3,4-dihydroisoquinolin-1(2H)-one (A1-7) (1 kg (from total 1.65 kg of compound A1-6), yield 67.2%, from total 1.65 kg of compound A1-6) as white solid. LCMS: [M+H]⁺=256.2.

Step 7: To a suspension of compound A1-7 (195 g, 0.76 mol, 1.0 eq) in dichloromethane/tetrahydrofuran (2.1 L/0.7 L) were added DIEA (2 mL) and SOCl₂ (906 g, 550 mL, 7.614 mol, 10.0 eq). The mixture was stirred at 25° C. for 1 hr and then concentrated to afford 5-bromo-7-(chloromethyl)-3,4-dihydroisoquinolin-1(2H)-one (A1-8) (240 g, crude) as a yellow solid, which was used in the next step directly.

Step 8: To a suspension of compound A1-8 (240 g, 0.76 mol, 1.0 eq, crude) and N-methyl-1H-imidazol-2-amine (140 g, 0.83 mol, 1.1 eq. crude) in dimethylformamide (2.5 L) was added sodium carbonate (323 g, 3.0456 mol, 4.0 eq.). The mixture was stirred at 60-65° C. for 18 hrs under N₂ atmosphere. LCMS showed 91% completion. The reaction mixture was filtered and the cake was washed with dimethyl formamide (3 L). The filtrate was concentrated under reduced pressure. Ethyl acetate/methanol (700 mL/80 mL) was added to the residue. The mixture was stirred for 1 h. and filtered. The solid was dried in vacuum to give 5-bromo-7-((2-(methylamino)-1H-imidazol-1-yl)methyl)-3,4-dihydroisoquinolin-1(2H)-one (A1) (154 g, 60.3% overall two steps) as a gray solid. ¹H NMR (400 MHz, Methanol-d₄) δ 7.79 (d, J=1.4 Hz, 1H), 7.50 (d, J=1.6 Hz, 1H), 6.64 (d, J=1.6 Hz, 1H), 6.60 (d, J=1.6 Hz, 1H), 4.96 (s, 2H), 3.49 (t, J=6.7 Hz, 2H), 3.05 (t, J=6.8 Hz, 2H), 2.86-2.84 (m, 3H). LCMS: [M+H]⁺=335.2.

### Intermediate A2: 5-chloro-7-((2-(methylamino)-1H-imidazol-1-yl)methyl)-3,4-dihydroisoquinolin-1(2H)-one (A2)

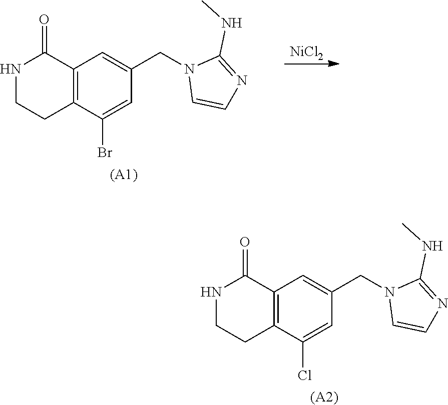

Intermediate (A1) (200 mg, 0.597 mmol), nickel(II) chloride (155 mg, 1.193 mmol), and DMF (Volume: 4 mL) were added to a microwave tube. Then the mixture was heated and stirred at 170° C. for 7 min by microwave reactor (Biotage 356004). The reaction mixture was cooled to room temperature and filtered. The filtrate was purified by preparative-HPLC (Column: Luna C18 150*25 5 u, gradient: 15-42% of B, A=water (0.225% FA)-ACN, B=acetonitrile, flow rate: 100 mL/min) to give 5-chloro-7-((2-(methylamino)-1H-imidazol-1-yl)methyl)-3,4-dihydroisoquinolin-1(2H)-one (A2) (120 mg, 69.2%) as light yellow solid. LCMS: [M+H]⁺=291.0.

### Intermediate A3: 7-((2-(methylamino)-1H-imidazol-1-yl)methyl)-3,4-dihydroisoquinolin-1(2H)-one (A3)

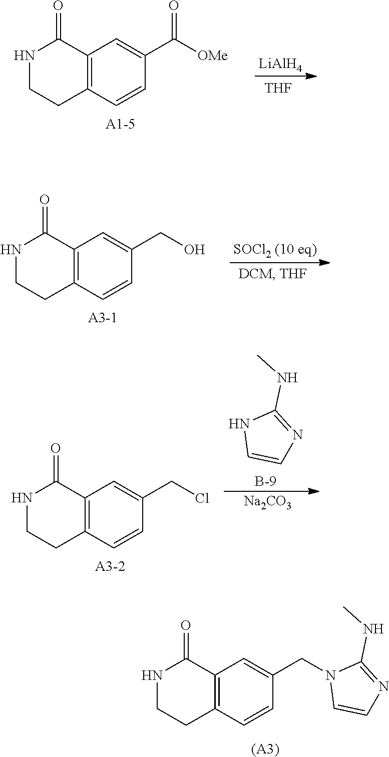

Step 1: To a mixture of compound A1-5 (30 g, 146.2 mmol, 1.0 eq) in THF (300 mL) was added LiAlH₄ (8.32 g, 219.3 mmol, 1.5 eq) in portions at 0° C. The mixture was stirred at 0-5° C. for 3 h. The reaction was quenched with sat. NH₄Cl (200 mL). The liquid layer was extracted with DCM (1000 mL×2). The combined organic layer was concentrated. To the residue was added ethyl acetate (100 mL) and stirred for 30 mins then filtered. The solid was dried in vacuum to give compound A3-1 (20.5 g, 115.7 mmol, 79.1% of yield) as a grey solid. ¹HNMR (400 MHz, METHANOL-d₄) δ ppm 2.97 (t, J=6.60 Hz, 2H) 3.44-3.55 (m, 2H) 4.63 (s, 2H) 7.29 (d, J=7.83 Hz, 1H) 7.46-7.53 (m, 1H) 7.93 (s, 1H). LCMS MS (ESI) m/z 178.2 [M+H]⁺.

Step 2: To a mixture of compound A3-1 (20.5 g, 115.7 mmol, 1.0 eq) in DCM/THF (300 mL/100 mL) were added SOCl₂ (137.7 g, 1157 mmol, 10.0 eq) and DIPEA (1 mL). The mixture was stirred at 10-15° C. for 3 hours. The mixture was concentrated to afford compound A3-2 (11.3 g) as a black solid which was used for the next step directly. LCMS MS (ESI) m/z 196.1 [M+H]+.

Step 3: To a mixture of compound A3-2 (10.2 g, crude, 38.5 mmol, 1.0 eq) in DMF (150 mL) were added N-methyl-1H-imidazol-2-amine (6.3 g, crude, 38.5 mmol, 1.0 eq) and Na₂CO₃ (12.24 g, 115.5 mmol, 3.0 eq). The mixture was stirred at 80° C. for 18 h. Methanol/DCM (40 mL/400 mL) was added and the solution was stirred at 25° C. for 1 h. The solution was filtered and the filtrate was concentrated to afford the crude product. The crude was purified by column chromatography eluting with ethyl acetate/methanol (gradient: 0%˜10% of methanol) to afford 7-((2-(methylamino)-1H-imidazol-1-yl)methyl)-3,4-dihydroisoquinolin-1(2H)-one (A3) (6 g, 48% yield) as a red solid. ¹HNMR (400 MHz, Methanol-d₄) δ ppm 7.74 (s, 1H), 7.26-7.18 (m, 2H), 6.59-6.57 (m, 1H), 6.57-6.55 (m, 1H), 4.92 (s, 2H), 3.47-3.39 (m, 2H), 2.91 (t, J=6.7 Hz, 2H), 2.85-2.78 (m, 4H). LC-MS: [M+H]⁺=257.2.

### Intermediate B1: 5-bromo-7-(2-(ethyl(methyl)amino)ethyl)-3,4-dihydroisoquinolin-1(2H)-one (B1)

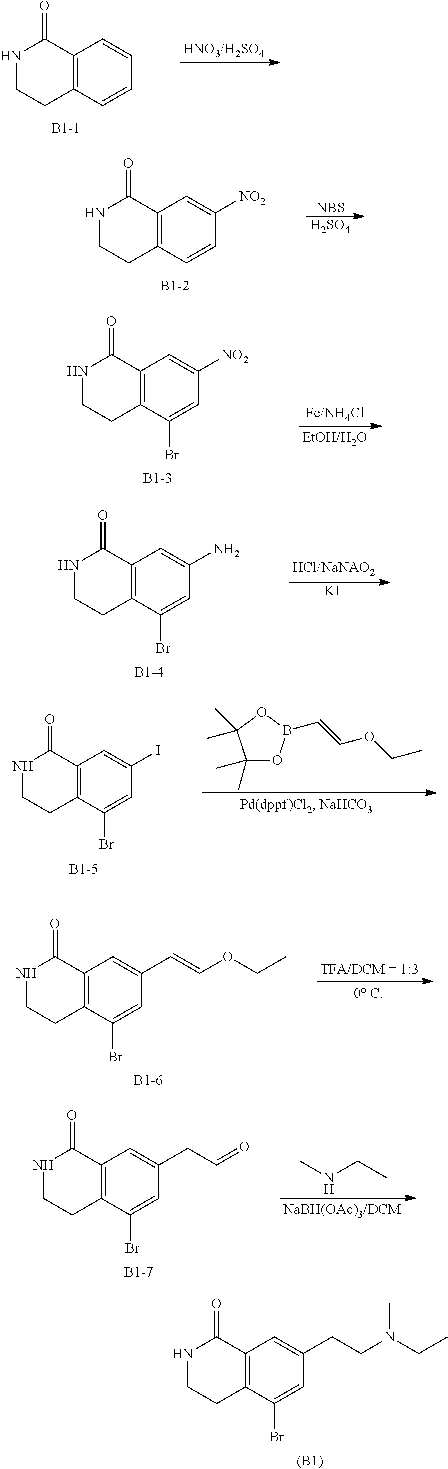

Step 1: To a mixture of fuming HNO₃ (300 mL) and conc. H₂SO₄ (1.2 L) was added compound B1-1 (230 g, 1.563 mol, 1 equiv) in portions at 0° C. The reaction mixture was stirred at 25° C. for 1 hour. TLC (100% EA) confirmed compound B1-1 was consumed. The reaction mixture was poured into ice water (10 L) and the solid was collected by filtration to afford 7-nitro-3,4-dihydroisoquinolin-1(2H)-one (B1-2) (360 g, crude) as a yellow solid. ¹H NMR (400 MHz, DMSO-d₆) δ=8.53 (d, J=2.6 Hz, 1H), 8.30 (dd, J=2.6, 8.3 Hz, 2H), 7.62 (d, J=8.4 Hz, 1H), 3.45-3.41 (m, 2H), 3.05 (t, J=6.5 Hz, 2H).

Step 2: To a solution of compound B1-2 (360 g, crude, 1.563 mol, 1 equiv) in conc. H₂SO₄ (1.8 L) was added NBS (334 g, 1.876 mol, 1.2 equiv) in portions at 60° C. The reaction mixture was stirred at 60° C. for 1 hr. The reaction mixture was poured into ice water (12 L) and the solid was collected by filtration to afford 5-bromo-7-nitro-3,4-dihydroisoquinolin-1(2H)-one (B1-3) (330 g, crude) as a yellow solid. ¹H NMR (400 MHz, DMSO-d₆) δ=8.59-8.51 (m, 2H), 8.43 (br, 1H), 3.50-3.45 (m, 2H), 3.08 (t, J=6.6 Hz, 2H).

Step 3: To a solution of compound B1-3 (170 g, 627 mmol, crude) in ethanol (1600 mL) and water (400 mL) was added iron powder (175 g, 3136 mmol, 5 equiv), NH₄Cl (33.5 g, 627 mmol, 1 equiv) at 25° C. The reaction mixture was stirred at 70° C. for 2 hrs. The mixture was filtered through Celite without cooling. The solid was washed with ethanol. The filtration was combined with another batch (from 170 g of compound B1-3) and concentrated under reduced pressure. The residue was adjusted to pH-8 with sat.NaHCO₃. The solid was filtered, washed with ethyl acetate (200 mL×3) and collected. The filtration was extracted with ethyl acetate (200 mL×3). The ethyl acetate was concentrated under reduced pressure. The residue was crystalized by ethyl acetate (100 mL). The solid was combined together to afford 7-amino-5-bromo-3,4-dihydroisoquinolin-1(2H)-one (B1-4) (270 g, 1120.0 mmol, 70% yield, three steps). ¹H NMR (400 MHz, DMSO-d₆) δ 7.95 (br, 1H), 7.20 (d, J=2.3 Hz, 1H), 7.01 (d, J=2.2 Hz, 1H), 5.49 (s, 2H), 3.36 (m, 2H), 2.81 (m, 2H).

Step 4: To a solution of compound B1-4 (20 g, 83 mmol, 1 equiv) in conc. HCl (267 mL) was added a solution of NaNO₂ (6.3 g, 91 mmol, 1.1 equiv) in water (80 mL) at 0° C. dropwise (keep the temperature below 5° C.). The reaction mixture was stirred at 0° C. for 1 hr and then the solution of KI (41.3 g, 248.9 mmol, 3 equiv) in water (80 mL) was added dropwise at 0° C. (keep the temperature below 5° C.). The reaction mixture was stirred at 0° C. for 30 mins then at 20° C. for 3 hrs. LCMS showed 88.5% of desired MW was detected. The reaction mixture was diluted with water (250 mL) and filtered. The solid was washed with aq.Na₂SO₃ (200 mL). The cake was washed with water (500 mL). The solid was dried to afford 5-bromo-7-iodo-3,4-dihydroisoquinolin-1(2H)-one (B1-5) (21 g, 71.9% yield) as yellow solid. ¹H NMR (400 MHz, DMSO-d₆) δ 8.20 (br, 1H), 8.13 (d, J=5.7 Hz, 2H), 3.38 (dt, J=2.7, 6.6 Hz, 2H), 2.89 (t, J=6.6 Hz, 2H).

Step 5: To a solution of compound B1-5 (100 g, 284 mmol, 1 equiv, crude) in dioxane (1200 mL) and water (300 ml) was added sodium hydrogencarbonate (83.6 g, 994 mmol, 3 equiv), (E)-2-(2-ethoxyvinyl)-4,4,5,5-tetramethyl-1,3,2-dioxaborolane (76 g, 284 mmol, 1 equiv, 70% purity), PdCl₂(dppf)DCM (14 g, 17.04 mmol, 0.06 equiv) at 25° C. under nitrogen atmosphere. The reaction mixture was stirred at 80° C. for 3 hrs under nitrogen atmosphere. The reaction was concentrated. The residue was purified by combi-flash (Gradient: 0-60% of B, A=petroleum ether, B=ethyl acetate) to give (E)-5-bromo-7-(2-ethoxyvinyl)-3,4-dihydroisoquinolin-1(2H)-one (B1-6) (120 g, 72% yield) as light yellow solid. 1H NMR (400 MHz, Methanol-d4) δ 7.82 (d, J=1.8 Hz, 1H), 7.65 (d, J=1.9 Hz, 1H), 7.19 (d, J=12.9 Hz, 1H), 5.82 (d, J=12.9 Hz, 1H), 3.92 (q, J=7.0 Hz, 2H), 3.52-3.43 (m, 2H), 3.01 (t, J=6.7 Hz, 2H), 1.31 (t, J=7.0 Hz, 3H).

Step 6: To a solution of compound B1-6 (40 g, 135.1 mmol, 1 equiv) in dichloromethane (1800 mL) was added TFA (300 mL) at 0° C. The reaction mixture was stirred at 0° C. for 1 hour. LCMS showed the reaction was complete. The reaction mixture was quenched with aq.NaHCO₃ and adjusted to about pH 8. The aqueous layer was extracted with dichloromethane (200 mL). The combined organic phase was washed with brine (500 mL), dried over Na₂SO₄, and filtered to afford a solution of 2-(5-bromo-1-oxo-1,2,3,4-tetrahydroisoquinolin-7-yl)acetaldehyde (B1-7) in dichloromethane (2000 mL), which was used in the next step directly.

Step 7: The solution of compound B1-7 (135.1 mmol, crude, 2 L in dichloromethane) was diluted with dichloromethane (200 mL). To this solution was added N-methylethanamine (24 g, 405.3 mmol, 3 equiv) at 20° C. The reaction mixture was stirred at 20° C. for 2 hours. Then NaBH(OAc)₃ (71.6 g, 337.8 mmol, 2.5 equiv) was added portion wise at 20° C. The reaction mixture was stirred at 20° C. for 16 hours. The reaction mixture was quenched with aq. HCl (2M) and adjusted to about pH 3 and diluted with water (1.5 L). The organic layer was abandoned. The aqueous layer was adjusted to pH=8 with aq. Na₂CO₃. Then the aqueous layer was extracted with ethyl acetate (600 mL×3). The organic layers were washed with brine (1 L), dried over Na₂SO₄, filtered and concentrated to give 5-bromo-7-(2-(ethyl(methyl)amino)ethyl)-3,4-dihydroisoquinolin-1(2H)-one (B1) (30 g, 71.4% yield) as yellow oil. ¹H NMR (400 MHz, Methanol-d4) δ 7.83 (d, J=1.7 Hz, 1H), 7.66 (d, J=1.7 Hz, 1H), 3.53-3.47 (m, 2H), 3.04 (t, J=6.7 Hz, 2H), 2.86-2.79 (m, 2H), 2.69-2.62 (m, 2H), 2.55 (q, J=7.2 Hz, 2H), 2.32 (s, 3H), 1.10 (t, J=7.2 Hz, 3H).

### Intermediate C1: 6-(1-(5-Bromo-7-((2-methyl-1H-imidazol-1-yl)methyl)-1-oxo-3,4-dihydroisoquinolin-2(1H)-yl)ethyl)-4-ethoxynicotinonitrile (C1)

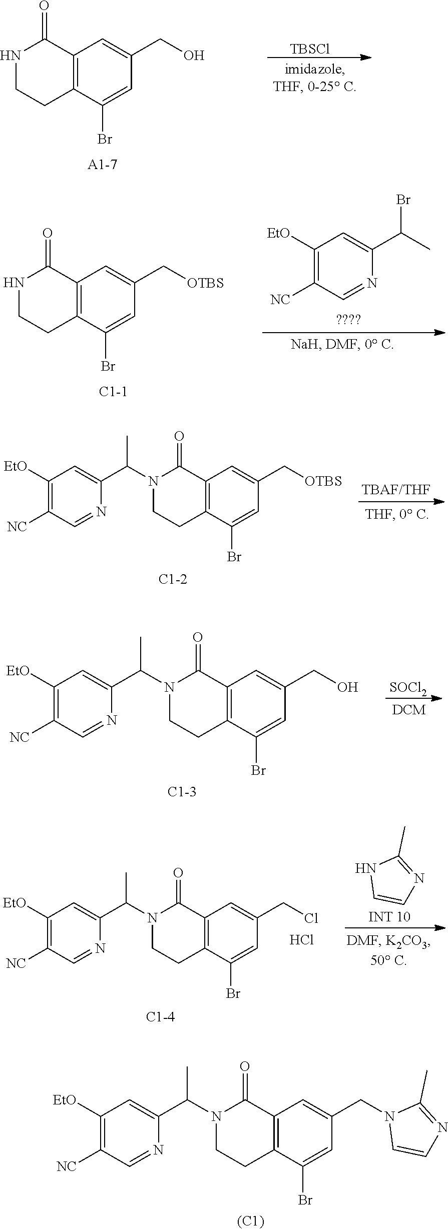

Step 1: To a solution of 5-bromo-7-(hydroxymethyl)-3,4-dihydroisoquinolin-1(2H)-one (A1-7) (18.0 g, 70.3 mmol) in THF (2.0 L) was added imidazole (23.9 g, 351.5 mmol, 5 eq.). TBSCl (31.7 g, 210.8 mmol, 3 eq.) was then added portion wise at 0° C. and the reaction mixture was allowed to warm up to 25° C. and was then stirred overnight. The reaction mixture was diluted with water (2.0 L) and the mixture was extracted with ethyl acetate (1 L×2). The combined organic layers were dried over anhydrous Na₂SO₄, concentrated to give a residue which was purified by column chromatography on silica gel (petroleum ether/ethyl acetate=10:1 to 1:1) to give 5-Bromo-7-(((tert-butyldimethylsilyl)oxy)methyl)-3,4-dihydroisoquinolin-1(2H)-one (C1-1), 24.9 g, 96% yield) as a white solid. ¹H NMR (400 MHz, DMSO-d6) δ 7.99 (s, 1H), 7.76 (s, 1H), 7.59 (s, 1H), 4.64 (s, 2H), 3.31 (m, 2H), 2.86 (m, 2H), 0.82 (s, 9H), 0.01 (s, 6H).

Step 2: To a solution of compound C1-1 (30.0 g, 81 mmol) in DMF (500 mL) was added NaH (60% in mineral oil, 4.86 g, 121.5 mol, 1.5 eq.) at 0° C. and the mixture was stirred at 0° C. for 1.5 hours. 6-(1-Bromoethyl)-4-ethoxynicotinonitrile (G1) (22.7 g, 89.1 mmol, 1.1 eq.) was added portion wise to the mixture at 0-5° C. and the reaction mixture was stirred for 1.5 hours. The mixture was diluted in aqueous NH₄Cl solution (10%, 1 L) and then brine (1 L) was added to the mixture. The mixture was extracted with ethyl acetate (1 L×3) and the combined organic layers were dried over anhydrous Na₂SO₄, concentrated to dryness to give a residue. The residue was purified by column chromatography on silica gel (petroleum ether/ethyl acetate=10:1 to 3:1 to give 6-(1-(5-bromo-7-(((tert-butyldimethylsilyl)oxy)methyl)-1-oxo-3,4-dihydroisoquinolin-2(1H)-yl)ethyl)-4-ethoxynicotinonitrile (C1-2), 40.9 g, 93% yield) as a white solid. ¹H NMR (400 MHz, DMSO-d6) δ 8.67 (s 1H), 7.82 (s, 1H), 7.60 (s, 1H), 7.12 (s, 1H), 5.80 (m, 1H), 4.65 (s, 2H), 4.25 (m, 2H), 3.44 (m, 1H), 3.32 (m, 1H), 2.89 (m, 2H), 1.49 (d, J=6.8 Hz, 3H), 1.27 (m, 3H), 0.81 (s, 9H), 0.03 (s, 6H).

Step 3: To a solution of compound C1-2 (75.2 mmol) in THF (300 mL) was added TBAF (1.0 M in THF, 188.2 mL, 188.2 mmol, 2.5 eq.) at 0° C. and the reaction mixture was stirred at 0° C. for 1 hour. The solution was diluted in ice water (1.5 L) and the mixture was extracted with ethyl acetate (300 mL×3). The combined organic layers were dried over anhydrous Na₂SO₄, concentrated to dryness to give a residue. The residue was purified by column chromatography on silica gel (petroleum ether/ethyl acetate=10:1 to pure EA) to give 6-(1-(5-Bromo-7-(hydroxymethyl)-1-oxo-3,4-dihydroisoquinolin-2(1H)-yl)ethyl)-4-ethoxynicotinonitrile (C1-3) (25.9 g, 80% yield) as a white solid. ¹H NMR (400 MHz, DMSO-d6) δ 8.82 (s, 1H), 7.95 (s, 1H), 7.75 (s, 1H), 7.26 (s, 1H), 5.97 (m, 1H), 5.44 (m, 1H), 4.58 (m, 2H), 4.39 (m, 2H), 3.61 (m, 1H), 3.47 (m, 1H), 3.04 (m, 2H), 1.63 (d, J=7.2 Hz, 3H), 1.44 (m, 3H). LCMS: (ESI) m/z 431.8 [M+H]⁺.

Step 4: To a solution of compound (C1-3) (12 g, 27.8 mmol) in DCM (100 mL) was added SOCl₂ (9.86 g, 83.3 mmol, 3.0 eq.) at −10° C. and the reaction mixture was allowed to warm up to 20° C. and stirred for 30 minutes. The mixture was concentrated to dryness to give 6-(1-(5-Bromo-7-(chloromethyl)-1-oxo-3,4-dihydroisoquinolin-2(1H)-yl)ethyl)-4-ethoxynicotinonitrile hydrochloride (C1-4) (7.0 g crude, 100% yield) as a light yellow thick oil, which was directly carried over to the next step without further purification.

Step 5: To a solution of compound (C1-4) (13.5 g, 27.8 mmol) in DMF (150 mL) was added 2-methyl-1H-imidazole (4.5 g, 55.6 mmol, 2 eq.) and K₂CO₃ (19.2 g, 138 mmol, 5 eq.) and the reaction mixture was stirred at 50° C. overnight. The solid was filtered off and the filtrate was concentrated to dryness to give a residue. The residue was purified by Prep. HPLC (Phenomenex Synergi Max-RP 250*80 mm*10 um, water (10 mM NH₄HCO₃)—CH₃CN) and dried by lyophilization to give 6-(1-(5-Bromo-7-((2-methyl-1H-imidazol-1-yl)methyl)-1-oxo-3,4-dihydroisoquinolin-2(1H)-yl)ethyl)-4-ethoxynicotinonitrile (C1) (11.3 g, 82% yield) as a white solid. ¹H NMR (400 MHz, DMSO-d6) δ 8.76 (s, 1H), 7.69 (d, J=1.6 Hz, 1H), 7.59 (d, J=2.0 Hz, 1H), 7.20 (s, 1H), 7.16 (s, 1H), 6.79 (s, 1H), 5.87 (m, 1H), 5.21 (s, 2H), 4.27 (q, J=6.9 Hz, 2H), 3.57 (m, 1H), 3.40 (m, 1H), 2.96 (m, 2H), 2.25 (s, 3H), 1.57 (d, J=6.8 Hz, 3H), 1.36 (t, J=7.0 Hz, 3H).

### Intermediate C2: 6-((5-bromo-7-((2-methyl-1H-imidazol-1-yl)methyl)-1-oxo-3,4-dihydroisoquinolin-2(1H)-yl)methyl)-4-ethoxynicotinonitrile (C2)

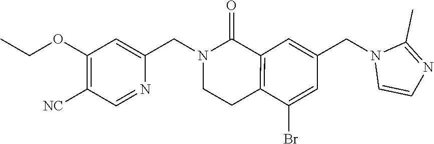 [SMILES: CCOc1cc(CN2CCc3c(Br)cc(Cn4ccnc4C)cc3C2=O)ncc1C#N]

The procedure for the synthesis of intermediate (C2) was similar to the synthesis of intermediate (C1), however in step 2, 6-(1-bromoethyl)-4-ethoxynicotinonitrile (G1) was replaced with 6-(bromomethyl)-4-ethoxynicotinonitrile (G2). 6-((5-bromo-7-((2-methyl-1H-imidazol-1-yl)methyl)-1-oxo-3,4-dihydroisoquinolin-2(1H)-yl)methyl)-4-ethoxynicotinonitrile (C2): (HCO₂H salt:) ¹H NMR (400 MHz, DMSO-d6) δ 8.60 (s, 1H), 8.33 (s, 1H), 7.91 (s, 1H), 7.40 (s, 1H), 7.12 (s, 1H), 7.01 (s, 1H), 6.91 (s, 1H), 5.09 (s, 2H), 4.84 (s, 2H), 4.23 (m, 2H), 3.76 (m, 2H), 3.12 (m, 2H), 2.49 (s, 3H), 1.52 (m, 3H). LC-MS: [M+H]⁺=257.2. (ESI) m/z 480.0 [M+H]+.

### Intermediate C3: 5-bromo-2-(1-(4-ethoxy-5-fluoropyridin-2-yl)ethyl)-7-((2-methyl-1H-imidazol-1-yl)methyl)-3,4-dihydroisoquinolin-1(2H)-one (C3)

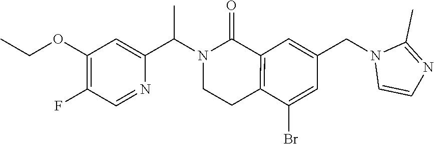 [SMILES: CCOc1cc(C(C)N2CCc3c(Br)cc(Cn4ccnc4C)cc3C2=O)ncc1F]

The procedure for the synthesis of intermediate (C3) was similar to the synthesis of intermediate (C1), however in step 2, 6-(1-bromoethyl)-4-ethoxynicotinonitrile (G1) was replaced with 2-(1-bromoethyl)-4-ethoxy-5-fluoropyridine (G5). 5-bromo-2-(1-(4-ethoxy-5-fluoropyridin-2-yl)ethyl)-7-((2-methyl-1H-imidazol-1-yl)methyl)-3,4-dihydroisoquinolin-1(2H)-one (C3): ¹H NMR (400 MHz, DMSO-d6) δ 8.37 (d, J=3.2 Hz, 1H), 7.70 (s, 1H), 7.58 (s, 1H), 7.16 (m, 2H), 6.79 (m, 1H), 5.87 (m, 1H), 5.21 (s, 2H), 4.27 (q, J=6.8 Hz, 2H), 3.49 (m, 1H), 3.31 (m, 1H), 2.94 (m, 2H), 2.25 (s, 3H), 1.53 (d, J=6.8 Hz, 3H), 1.36 (m, 3H). LC-MS: (ESI) m/z 488.36 [M+H]⁺.

### Intermediate C4: 6-(1-5-bromo-7-(2,4-dimethyl-1H-imidazol-1-yl)methyl)-1-oxo-3,4-dihydroisoquinolin-2(1H)-yl)ethyl)-4-ethoxynicotinonitrile (C4)

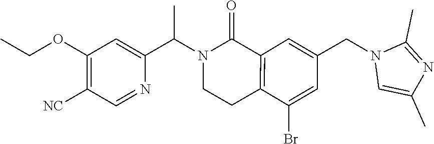 [SMILES: CCOc1cc(C(C)N2CCc3c(Br)cc(Cn4cc(C)nc4C)cc3C2=O)ncc1C#N]

The procedure for the synthesis of intermediate (C3) was similar to the synthesis of intermediate (C1), however in step 5, 2-methyl-1H-imidazole was replaced with 2,4-dimethyl-1H-imidazole. 6-(1-(5-bromo-7-((2,4-dimethyl-1H-imidazol-1-yl)methyl)-1-oxo-3,4-dihydroisoquinolin-2(1H)-yl)ethyl)-4-ethoxynicotinonitrile (C4): ¹H NMR (400 MHz, CD₃OD) δ 8.62 (d, J=1.7 Hz, 1H), 7.74 (d, J=1.9 Hz, 1H), 7.53 (t, J=1.8 Hz, 1H), 7.16 (s, 1H), 6.75 (t, J=1.4 Hz, 1H), 5.91 (q, J=7.1 Hz, 1H), 5.12 (s, 2H), 4.30 (q, J=7.0 Hz, 2H), 3.75-3.47 (m, 2H), 3.15-2.93 (m, 2H), 2.26 (d, J=1.1 Hz, 3H), 2.12 (t, J=1.1 Hz, 3H), 1.65 (d, J=7.1 Hz, 3H), 1.55-1.41 (m, 3H). LC-MS: (ESI) m/z 508.0 [M+H]⁺.

### Intermediate C5: 6-(1-(5-bromo-7-((2-methyl-1H-imidazol-1-yl)methyl)-1-oxo-3,4-dihydroisoquinolin-2(1H)-yl)ethyl)-4-methoxynicotinonitrile (C5)

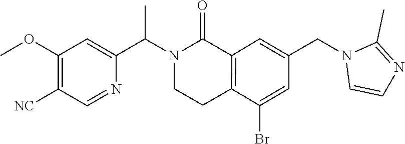 [SMILES: COc1cc(C(C)N2CCc3c(Br)cc(Cn4ccnc4C)cc3C2=O)ncc1C#N]

The procedure for the synthesis of intermediate (C3) was similar to the synthesis of intermediate (C1), however in step 2, 6-(1-bromoethyl)-4-ethoxynicotinonitrile (G1) was replaced with 6-(1-bromoethyl)-4-methoxynicotinonitrile (G12). 6-(1-(5-bromo-7-((2-methyl-1H-imidazol-1-yl)methyl)-1-oxo-3,4-dihydroisoquinolin-2(1H)-yl)ethyl)-4-methoxynicotinonitrile (C5): ¹H NMR (400 MHz, CD₃OD) δ 8.65 (s, 1H), 7.78 (s, 1H), 7.56 (s, 1H), 7.23 (s, 1H), 7.11 (d, J=1.6 Hz, 1H), 6.91 (d, J=1.5 Hz, 1H), 5.95 (d, J=7.2 Hz, 1H), 5.25 (s, 2H), 4.06 (s, 3H), 3.63 (q, J=7.3 Hz, 2H), 3.17-3.04 (m, 2H), 2.33 (s, 3H), 1.68 (d, J=7.1 Hz, 3H). LC-MS: (ESI) m/z 480.1 [M+H]⁺.

### Intermediate D1: methyl 5-bromo-1-oxo-1,2-dihydroisoquinoline-7-carboxylate (D1)

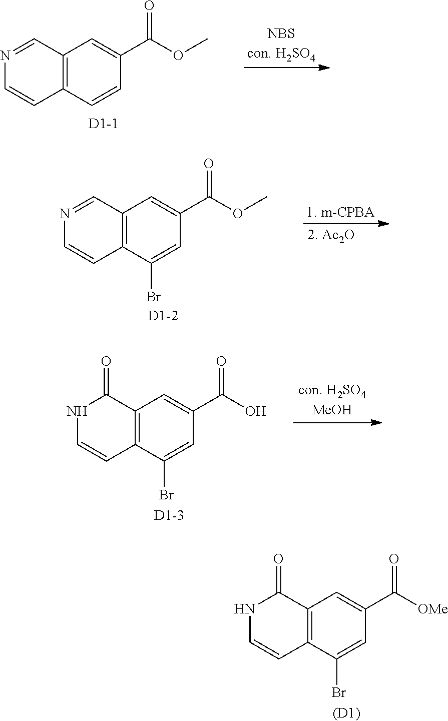

Step 1: To a solution of methyl isoquinoline-7-carboxylate (D1-1) (7.2 g, 38.5 mmol) in con.H₂SO₄ (60 mL) was added NBS (10.3 g, 57.8 mmol) in portions at 5-10° C. under nitrogen atmosphere. The mixture was stirred at 4-9° C. for 18 hours under nitrogen atmosphere. The mixture was poured into ice water (500 mL) and the aqueous solution was adjusted to pH 9-10 with ammonium in an ice bath. The suspension was filtered and the filter cake was washed with H₂O (100 mL) and MTBE (150 mL) in sequence. The crude was dissolved in dichloromethane (300 mL) and the organic layer was separated, dried and concentrated, then purified by flash column (petroleum ether/EtOAc=20/1 to petroleum ether/dichloromethane/EtOAc=10/10/1) to afford methyl 5-bromoisoquinoline-7-carboxylate (D1-2) (6.1 g, 60% yield) as a light yellow solid. ¹H NMR (400 MHz, d⁶-DMSO) δ 9.59 (s, 1H), 8.89 (s, 1H), 8.80 (d, J=6.0 Hz, 1H), 8.47 (d, J=1.2 Hz, 1H), 8.00 (d, J=6.0 Hz, 1H), 3.96 (s, 3H).

Step 2: To a solution of compound D1-2 (6 g, 22.6 mmol) in dichloromethane (150 mL) was added m-CPBA (5.83 g, 33.9 mmol) in portions in an ice bath under nitrogen atmosphere. The mixture was stirred at 2-8° C. for 4 hours. The mixture was quenched with sat NaHCO₃ (200 mL) and extracted with dichloromethane (200 mL×2). The combined organic layers were washed with sat. Na₂SO₃ (100 mL) and brine (100 mL) in sequence, dried and concentrated. The residue was added Ac₂O (100 mL) and then the mixture was stirred at 160° C. for 2 hour. The mixture was concentrated and of 2M NaOH (300 mL) was added. The mixture was heated up to 110° C. and stirred at 110° C. for 3 hrs. Then the reaction was cooled to 10° C. and adjusted to pH 3-4 with 2M HCl aqueous. The suspension was filtered and the filter cake was washed with 110 mL of the mixed solvents (dichloromethane/methanol=10/1) and dried to afford 5-bromo-1-oxo-1,2-dihydroisoquinoline-7-carboxylic acid (D-3) (7.8 g, crude) as a grey solid. ¹H NMR (400 MHz, d⁶-DMSO) δ 13.42 (br. s., 1H), 11.79 (br. s., 1H), 8.73 (s, 1H), 8.36 (d, J=1.2 Hz, 1H), 7.49 (t, J=6.8 Hz, 1H), 6.70 (d, J=7.2 Hz, 1H)

Step 3: To a suspension of compound D1-3 (2.4 g, 8.95 mmol) in methanol (70 mL) was added con.H₂SO₄ (3 mL) dropwise. The mixture was heated to 90° C. and stirred for 18 hours. The reaction solution was cooled in an ice bath and the suspension was filtered. The filter cake was washed with water (100 mL×2), dried to afford methyl 5-bromo-1-oxo-1,2-dihydroisoquinoline-7-carboxylate (D1) (2.2 g, 87% yield) as a grey solid. ¹H NMR (400 MHz, d⁶-DMSO) δ 11.82 (br. s., 1H), 8.73 (s, 1H), 8.37 (d, J=1.2 Hz, 1H), 7.51 (t, J=6.8 Hz, 1H), 6.70 (d, J=7.2 Hz, 1H), 3.91 (s, 3H). LCMS: [M+H]⁺=283.8.

### Intermediate D2: methyl 4-oxo-3,4-dihydrophthalazine-6-carboxylate (D2)

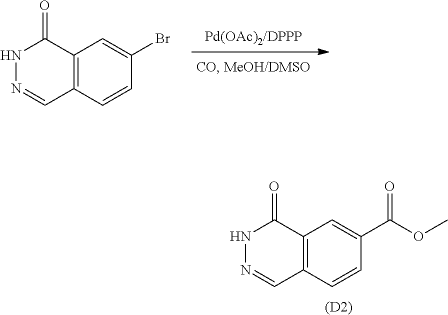

To a mixture of 7-bromophthalazin-1(2H)-one (500 mg, 2.2 mmol), triethylamine (0.67 g, 6.6 mmol) in methanol (9 mL) and DMSO (6 mL) was added Pd(OAc)₂ (100 mg, 0.44 mmol), DPPP (183 mg, 0.44 mmol) at 25° C. The reaction mixture was degassed and stirred under balloon pressure of CO gas atmosphere at 80° C. for 12 hours. The reaction mixture was filtered. The filtrate was concentrated and purified by purified by column chromatography (2-50% of ethyl acetate in Hexane) to afford methyl 4-oxo-3,4-dihydrophthalazine-6-carboxylate (D2) (300 mg, 66.8%) as a white solid. ¹H NMR (400 MHz, d⁶-DMSO) δ 12.89 (br s, 1H), 8.77-8.69 (m, 1H), 8.47 (s, 1H), 8.40 (dd, J=1.8, 8.2 Hz, 1H), 8.07 (d, J=8.2 Hz, 1H), 3.94 (s, 3H). LCMS: [M+H]⁺=205.1.

### Intermediate D3: methyl 4-oxo-3,4-dihydroquinazoline-6-carboxylate (D3)

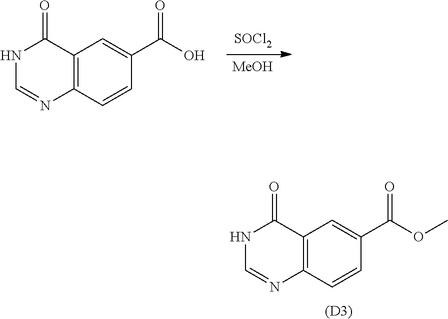

To a solution of 4-oxo-3,4-dihydroquinazoline-6-carboxylic acid (1.00 g, 5.26 mmol) in MeOH (40 mL) was added SOCl₂ (1.56 g, 13.15 mmol). The mixture was stirred at 80° C. for 16 hours. The reaction mixture was concentrated in vacuo, diluted with sat.NaHCO₃, extracted with EA/EtOH=5/1 (40 mL*5), the organic phase was concentrated to give methyl 4-oxo-3,4-dihydroquinazoline-6-carboxylate (D3) (850 mg, 79% yield) as a white solid. ¹H NMR (400 MHz, d⁶-DMSO) δ 8.68 (d, J=2.0 Hz, 1H), 8.29 (dd, J=1.9, 8.5 Hz, 1H), 8.23 (s, 1H), 7.76 (d, J=8.5 Hz, 1H), 3.91 (s, 3H).

### Intermediate E1: 5-bromo-2-(1-(4-ethoxy-5-isocyanopyridin-2-yl)ethyl)-7-(2-((2R,3R)-3-hydroxy-2-methylazetidin-1-yl)ethyl)-3,4-dihydroisoquinolin-1(2H)-one (E1)

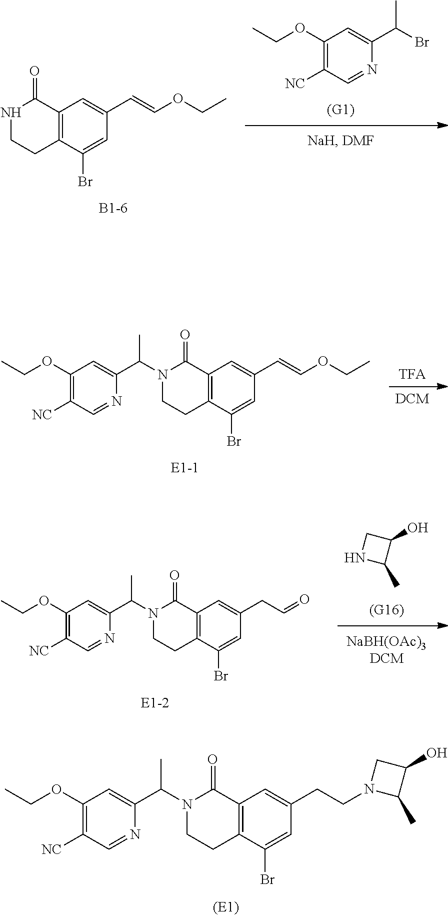

Step 1: To a solution of compound B1-6 (4 g, 13.50 mmol, 1.0 eq.) in DMF (80 mL) was added NaH (810 mg, 20.26 mmol, 1.5 eq, 60% purity in oil) at 0° C. under nitrogen atmosphere. The mixture was stirred at 0-5° C. for 0.5 hrs under nitrogen atmosphere and a solution of intermediate (G1) (4.13 g, 16.20 mmol, 1.2 eq.) in DMF (20 mL) was added dropwise. The reaction was warm to 25° C. and stirred for 1 hour. The mixture was quenched with methanol (20 mL) and diluted with water/brine (150 mL/150 mL). The solution was extracted with ethyl acetate (150 mL×3). The combined organic layers were washed with brine (100 mL×2), dried over anhydrous Na₂SO₄, filtered and concentrated. The residue was purified by Biotage-flash (Gradient: 20-80% of B, A=petroleum ether, B=ethyl acetate) to give (E)-6-(1-(5-bromo-7-(2-ethoxyvinyl)-1-oxo-3,4-dihydroisoquinolin-2(1H)-yl)ethyl)-4-ethoxynicotinonitrile (E1-1) (5 g, 78.7% yield) as yellow solid. ¹H NMR (400 MHz, CD₃OD): δ 8.63-8.57 (m, 1H), 8.10 (d, J=1.5 Hz, 1H), 7.96 (d, J=1.6 Hz, 1H), 7.15 (s, 1H), 6.36 (d, J=7.0 Hz, 1H), 5.97-5.87 (m, 1H), 5.18 (d, J=7.0 Hz, 1H), 4.28 (q, J=7.0 Hz, 2H), 4.03 (q, J=7.1 Hz, 2H), 3.65-3.46 (m, 2H), 3.06-2.89 (m, 2H), 1.64 (d, J=7.2 Hz, 3H), 1.45 (t, J=7.0 Hz, 3H), 1.35 (t, J=7.0 Hz, 3H).

Step 2: To a solution of compound E1-1 (2 g, 4.252 mmol, 1.0 eq) in dichloromethane (80 mL) was added trifluoroacetic acid (12 mL) at 0° C. dropwise. The mixture was stirred at 0° C. for 1 hrs. The reaction solution was adjusted to pH=8˜9 with aq. NaHCO₃. The organic layer was dried over anhydrous Na₂SO₄, filtered and concentrated to afford 2-(5-bromo-2-(1-(4-ethoxy-5-isocyanopyridin-2-yl)ethyl)-1-oxo-1,2,3,4-tetrahydroisoquinolin-7-yl)acetaldehyde (E1-2) (2 g, crude) as a yellow solid, which was used directly without purification.

Step 3: A solution of compound E1-2 (2 g, crude, 4.252 mmol, 1.0 eq) and intermediate (G16) (1.6 g, crude, 6.378 mmol, 1.5 equiv) in dichloromethane (80 mL) was stirred at 25° C. for 2 hrs. NaBH(OAc)₃ (2.25 g, 10.63 mmol, 2.5 eq) was added and the mixture was stirred at 25° C. for 4 hours. The mixture was diluted with aq. NaHCO₃ (100 mL) and extracted with dichloromethane (50 mL×2). The organic layers were washed with brine (100 mL), dried over Na₂SO₄, filtered and concentrated and purified by Biotage-flash (Gradient: 0-20% of B, A=dichloromethane, B=methanol, 1% triethylamine) to give 5-bromo-2-(1-(4-ethoxy-5-isocyanopyridin-2-yl)ethyl)-7-(2-((2R,3R)-3-hydroxy-2-methylazetidin-1-yl)ethyl)-3,4-dihydroisoquinolin-1(2H)-one (E1) (1.2 g) as yellow solid. ¹H NMR (400 MHz, CD₃OD): δ 8.60 (s, 1H), 7.89 (s, 1H), 7.52 (d, J=1.1 Hz, 1H), 6.98 (s, 1H), 6.04 (q, J=7.1 Hz, 1H), 4.29 (br t, J=5.1 Hz, 1H), 4.20 (q, J=7.1 Hz, 2H), 3.65-3.45 (m, 2H), 3.40-3.29 (m, 2H), 3.17-3.09 (m, 1H), 3.07-2.95 (m, 2H), 2.92-2.81 (m, 2H), 2.76-2.66 (m, 2H), 2.62-2.52 (m, 1H), 1.63 (d, J=7.1 Hz, 3H), 1.48 (t, J=7.0 Hz, 3H), 1.22 (d, J=6.5 Hz, 3H).

### Intermediate F1: 5-bromo-2-(1-(4-ethoxy-5-isocyanopyridin-2-yl)ethyl)-7-(2-((2R,3R)-3-hydroxy-2-methylpyrrolidin-1-yl)ethyl)-3,4-dihydroisoquinolin-1(2H)-one (F1)

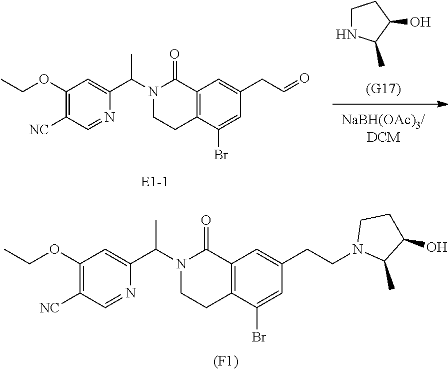

To a solution of compound E1-1 (370 mg, 0.85 mmol, 1 equiv) in dichloromethane (15 mL) was added intermediate (G17) (140 mg, 1.38 mmol, 1.6 equiv) at 25° C. The reaction mixture was stirred at 25° C. for 1.5 hours. Then NaBH(OAc)₃ (452 mg, 2.13 mmol, 2.5 equiv) was added portions at 25° C. The reaction mixture was stirred at 25° C. for 12 hours. The resulting mixture was adjusted to pH=8 by sat NaHCO₃ and extracted with ethyl acetate (20 mL×3). The combined organic phases were concentrated under reduced pressure to afford 5-bromo-2-(1-(4-ethoxy-5-isocyanopyridin-2-yl)ethyl)-7-(2-((2R,3R)-3-hydroxy-2-methylpyrrolidin-1-yl)ethyl)-3,4-dihydroisoquinolin-1(2H)-one (F1) (450 mg, crude) as a yellow oil. MS (ESI) m/z: 527/529 [M+H]+.

### Intermediate G1: 6-(1-bromoethyl)-4-ethoxynicotinonitrile (G1)

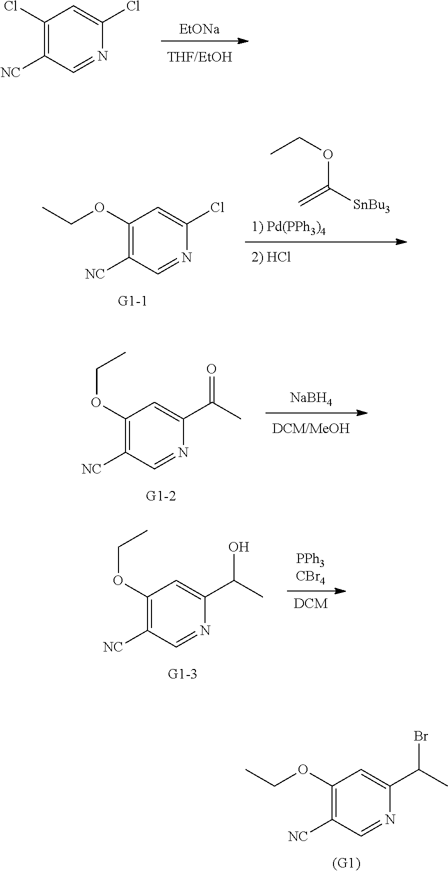

Step 1: The mixture of 4,6-dichloronicotinonitrile (25 g, 144 mmol) in THF (500 mL) was added a solution of EtONa (10.8 g, 158 mmol) in EtOH (100 mL) dropwise at 0° C. The filtrate was diluted with EA (200 mL), washed with brine (100 mL). The organic layer (THF and EA) was concentrated and purified by column chromatography (2˜50% B in Hexane) to give 6-chloro-4-ethoxynicotinonitrile (G1-1) (12 g, 45%). ¹H NMR (400 MHz, chloroform-d) δ=8.52-8.40 (m, 1H), 6.92 (s, 1H), 4.24 (q, J=7.0 Hz, 2H), 1.53 (t, J=7.0 Hz, 3H).

Step 2: To a mixture of G1-1 (11 g, 60.43 mmol) in toluene (120 mL) was added tributyl(1-ethoxyvinyl)stannane (36.96 g, 102.05 mmol), Pd(PPh₃)₄ (4.93 g, 4.27 mmol). The reaction was stirred at 110° C. for 8 hr under N₂, The reaction was cooled to 25° C. and concentrated to remove toluene then was added THF (60 mL), aq. HCl (2M, 60 mL), the resulting reaction was stirred at 25° C. for 3 hr. The mixture reaction was adjust to pH=7 with aq. NaHCO3 (2N, 100 mL), then the reaction mixture was extracted with EA (100 mL×3) and the combined organic layers were washed with 500 mL aq. KF, brine (200 mL), dried over Na2SO4. The organic layer was concentrated and purified by column chromatography (2˜16% ethyl acetate in Hexane) to afford 6-acetyl-4-ethoxynicotinonitrile (G1-2) (9.2 g, 79.4% yield) as white solid. ¹H NMR (CD₃OD 400 MHz): δ 8.78 (s, 1H), 7.70 (s, 1H), 4.39 (q, J=7.0 Hz, 2H), 2.67 (s, 3H), 1.51 (t, J=7.0 Hz, 3H).

Step 3: To a solution of G1-2 (4 g, 21.03 mmol) in MeOH (40 mL) and DCM (40 mL) was added NaBH₄ (0.967 g, 40.06 mmol) at −78° C. Then the mixture was warm to 0° C. and stirred at 0° C. for 0.5 hrs. The reaction was concentrated and purified by column chromatography (3˜30% ethyl acetate in hexane) to 4-ethoxy-6-(1-hydroxyethyl)nicotinonitrile (G1-3) (3.2 g, 80.1% yield) as a white solid. ¹H NMR (DMSO-d6 400 MHz): δ=8.70 (s, 1H), 7.30 (s, 1H), 5.62 (d, J=4.8 Hz, 1H), 4.77-4.69 (m, 1H), 4.37-4.26 (m, 2H), 1.42-1.38 (m, 3H), 1.36 (d, J=6.7 Hz, 3H). LCMS: MS (ESI) m/z 193.1 [M+H]⁺.

Step 4: To a solution of G1-3 (1.8 g, 9.37 mmol) and PPh₃ (3.68 g, 14.05 mmol) in dichloromethane (150 mL) was added CBr₄ (4.66 g, 14.05 mmol), The mixture was stirred at 25° C. for 18 hours. The mixture was concentrated and purified by column chromatography (2˜16% ethyl acetate in Hexane) to afford 6-(1-bromoethyl)-4-ethoxynicotinonitrile (G1) (1.72 g, 70.2% yield) as a colorless oil. ¹H NMR (CD₃OD 400 MHz): δ=8.62 (s, 1H), 7.04 (s, 1H), 5.15 (q, J=6.8 Hz, 1H), 4.28 (q, J=7.0 Hz, 2H), 2.05 (d, J=6.8 Hz, 3H), 1.54 (t, J=7.0 Hz, 3H). LCMS: MS (ESI) m/z 255.0 [M+H]⁺.

### Intermediate G2: 6-(bromomethyl)-4-ethoxynicotinonitrile (G2)

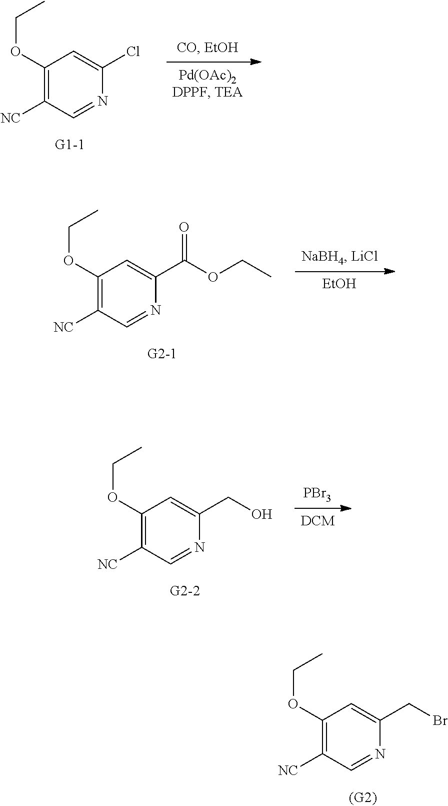

Step 1: To a solution of 6-chloro-4-ethoxynicotinonitrile (G1-1) (1 g, 5.48 mmol) in DMF (40 mL) and ethanol (40 mL) was added Pd(OAc)₂ (123.5 mg, 0.55 mmol), DPPF (609.8 mg, 1.1 mmol) and TEA (1.66 g, 16.44 mmol). The reaction was stirred at 50° C. for 24 hrs under CO (50 psi). The reaction mixture was diluted with water (300 mL) and extracted with ethyl acetate (120 mL×3). The combined organic layers were dried, concentrated, and purified by column chromatography (5˜20% of ethyl acetate in Hexane) to give ethyl 5-cyano-4-ethoxypicolinate (G2-1) (750 mg, 62.2% yield) as light yellow solid. ¹H NMR (CDCl₃ 400 MHz): δ 8.77 (s, 1H), 7.73 (s, 1H), 4.51 (q, J=7.1 Hz, 2H), 4.34 (q, J=7.0 Hz, 2H), 1.55 (t, J=7.0 Hz, 3H), 1.46 (t, J=7.2 Hz, 3H).

Step 2: To a solution of compound G2-1 (300 mg, 1.36 mmol) in ethanol (6 mL) was added LiCl (288.3 mg, 6.8 mmol) and NaBH₄ (257.2 mg, 6.8 mmol) at 0° C. under nitrogen atmosphere. The mixture was stirred at 20° C. for 3 hrs. The reaction was quenched with aq. NH₄Cl (20 mL) and H₂O (20 mL). The mixture was extracted with ethyl acetate (20 mL×3). The combined organic layers were washed with brine (30 mL), dried and concentrated to give 4-ethoxy-6-(hydroxymethyl)nicotinonitrile (G2-2) (240 mg, 99% yield) as white solid. LCMS: MS (ESI) m/z 179.0 [M+H]⁺.

Step 3: To a solution of G2-2 (240 mg, 1.347 mmol) in dichloromethane (40 mL) was added PBr₃ (584.7 mg, 2.16 mmol) at 0° C. under nitrogen atmosphere. The mixture was stirred at 35° C. for 2 hrs. The mixture was quenched by 15% aqueous ammonia solution and adjusted to pH=8. Then the mixture was diluted with water (30 mL). The aqueous layer was extracted with dichloromethane (30 mL×3). The combined organic layers were washed with brine (50 mL), dried and concentrated to give 6-(bromomethyl)-4-ethoxynicotinonitrile (G2) (330 mg, crude) as yellow solid. LCMS: MS (ESI) m/z 241.0 [M+H]⁺.

### Intermediate G3: 5-(1-bromoethyl)-3-ethoxypicolinonitrile (G3)

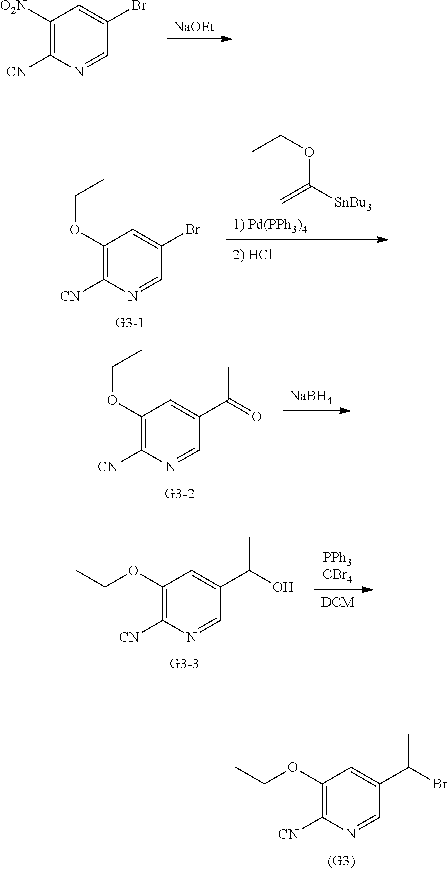

Step 1: To a flask was added 5-bromo-3-nitropicolinonitrile (912 mg, 4 mmol), EtONa (1.538 mL, 4.00 mmol) and EtOH (30 mL), then stirred at 90° C. for 16 h. Then solvent was removed, the residue was purified by flash column (30% EtOAc in hexane) to give 5-bromo-3-ethoxypicolinonitrile (G3-1) (610 mg, 67.2% yield). LC-MS: MS m/z 229.1 [M+H]⁺.

Step 2: Compound G3-1 (150 mg, 0.661 mmol) and Pd(PPh₃)₂Cl₂ (93 mg, 0.132 mmol) were added to a reaction tube and under N₂, 1,4-Dioxane (5 mL) and tributyl(1-ethoxyvinyl)stannane (0.253 mL, 0.727 mmol) were added. The mixture was then stirred at 100° C. for 18 hrs, cooled to rt, HCl (1.0M, 3 mL) was then added, and the mixture was stirred at rt for 3 hrs. The reaction was neutralized with Sat. NaHCO₃ (aq.), extracted with EtOAc, dried and concentrated, the residue was purified by flash column (30% EtOAc in Hexane) to give 5-acetyl-3-ethoxypicolinonitrile (G3-2) (103 mg, 82%) as yellow solid. LC-MS: MS m/z 191.2 [M+H]⁺.

Step 3: Compound G3-2 (50 mg, 0.263 mmol) and THF (3 mL) were added to a reaction tube, followed by NaBH4 (19.89 mg, 0.526 mmol), and the mixture was stirred at rt for 2 hrs. Then acetone (2 mL) was added, the solvent was evaporated, the residue was purified by flash column (50% EtOAc in Hexane) to give 3-ethoxy-5-(1-hydroxyethyl)picolinonitrile (G3-3) (32 mg, 63.3%). In LC-MS, MS m/z 193.2 [M+H]⁺.

Step 4: Compound G3-3 (31 mg, 0.161 mmol), PPh₃ (50.8 mg, 0.194 mmol) and DCM (2 mL) were added to a reaction tube and then under N₂ at 0° C., perbromomethane (66.9 mg, 0.202 mmol) in DCM (0.5) mL was added and the mixture was then stirred at rt for 15 hrs. The solvent was evaporated and the residue was purified by flash column (20% EtOAc in Hexane) to give product 5-(1-bromoethyl)-3-ethoxypicolinonitrile (G3) (18 mg, 43.7% yield) as white solid. LC-MS: MS m/z 255.1 [M+H]⁺.

### Intermediate G4: 5-(bromomethyl)-3-ethoxypicolinonitrile (G4)

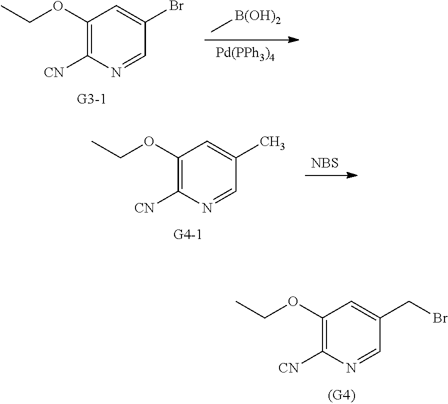

Step 1: 5-bromo-3-ethoxypicolinonitrile (G3-1) (510 mg, 2.25 mmol), methylboronic acid (269 mg, 4.49 mmol), Pd(Ph₃P)₄ (260 mg, 0.225 mmol) and Cs2CO3 (2195 mg, 6.74 mmol) were added to a reaction tube and then under N₂, 1,4-Dioxane (3.0 mL) and water (0.3 mL) were added. The mixture was then stirred at 110° C. for 16 hrs. Then the solvent was evaporated and the residue purified by flash column (30% EtOAc in Hexane) to 3-ethoxy-5-methylpicolinonitrile (G4-1) (310 mg, 85% yield) as yellow solid. LC-MS: MS m/z 163.1 [M+H]⁺.

Step 2: Compound G4-1 (220 mg, 1.356 mmol), 1-bromopyrrolidine-2,5-dione (266 mg, 1.492 mmol), and AIBN (22.27 mg, 0.136 mmol) were added to a reaction tube, followed by CCl4 (10 mL), and the mixture was then stirred at 80° C. for 18 hrs. The solvent was removed, and the residue was purified by flash column (30% EtOAc in Hexane) to give 5-(bromomethyl)-3-ethoxypicolinonitrile (G4) (120 mg, 36.7% yield) as yellow solid. LC-MS: MS m/z 240.9 [M+H]⁺.

### Intermediate G5: 2-(1-bromoethyl)-4-ethoxy-5-fluoropyridine (G5)

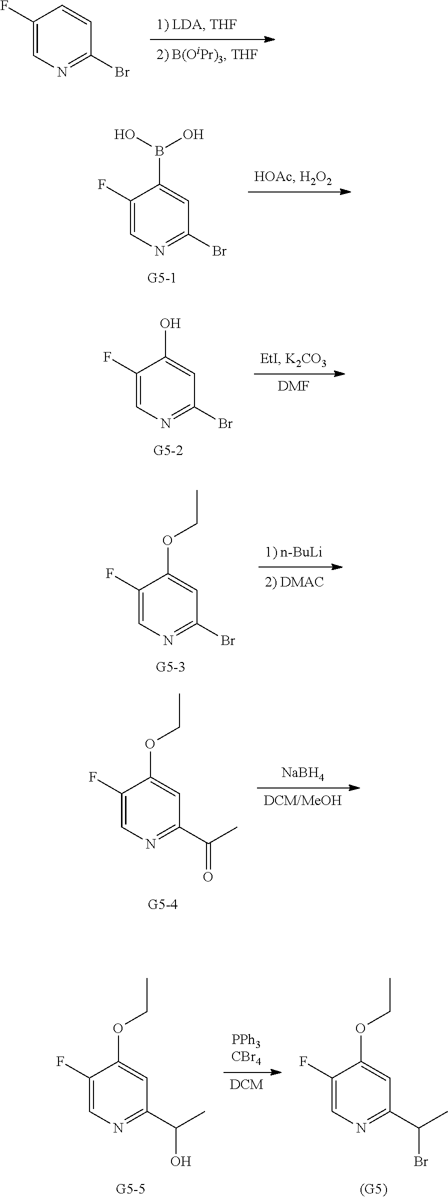

Step 1: To a solution of 2-bromo-5-fluoropyridine (400 g, 2.273 mol) in anhydrous tetrahydrofuran (4 L) was added LDA (1364 mL, 2.727 mol, 2 M in hexane) dropwise at −70° C. The mixture was stirred at −70° C. for 1 hr. Then a solution of B(OⁱPr)₃ (641 g, 3.409 mol) in anhydrous tetrahydrofuran (700 mL) was added dropwise to the above mixture at −70° C. The resulting mixture was stirred at −70° C. for 1 h. Aq.Na₂CO₃ solution (1000 g in 6 L water) was added to the above. The aqueous layer was separated, then acidified to pH 2 with HCl (6M) and then water added (3 L). The mixture was extracted with ethyl acetate (8 L×2). The combined organic layers were washed with brine (3 L), dried and concentrated to (2-bromo-5-fluoropyridin-4-yl)boronic acid (G5-1) (830 g, 83% yield) as a yellow solid. 1H NMR (CD₃OD 400 MHz) δ=8.29-8.12 (m, 1H), 7.64 (d, J=4.1 Hz, 1H)

Step 2: To a solution of compound G5-1 (500 g, 2.275 mol) in HOAc (2.5 L) was added H₂O₂ (700 mL, 30%) dropwise at 20-30° C. (caution: exothermic). The mixture was stirred at 25° C. for 1 hr. The mixture was poured into aq. Na₂SO₃ (1000 g in 5 L water) carefully and the resulting mixture was stirred at 25° C. for 0.5 h. The aqueous layer was extracted with ethyl acetate (4 L×3). The combined organic layers were washed with brine (1500 mL), dried and concentrated. To the residue was added methyl tertiary butyl ether (MTBE 600 mL) and stirred at 25° C. for 1 hr. The solid was gathered and dried in vacuum to 2-bromo-5-fluoropyridin-4-ol (G5-2) (420 g, 96% yield) as a yellow solid. ¹H NMR (CD₃OD 400 MHz) δ 8.04 (d, J=3.3 Hz, 1H), 7.06 (d, J=6.6 Hz, 1H).

Step 3: To a solution of compound G5-2 (400 g, 2.0834 mol) in DMF (5 L) was added K₂CO₃ (864 g, 6.25 mol). To the mixture was added Etl (585 g, 3.750 mmol) at 5-10° C. dropwise. The resulting mixture was stirred at 20-40° C. for 3 hrs. The reaction was diluted with water (20 L) and extracted with ethyl acetate (10 L×2). The combined organic layers were washed with brine (3 L), dried and concentrated to afford 2-bromo-4-ethoxy-5-fluoropyridine (G5-3) (368 g, 81% yield) as a pale yellow solid. ¹H NMR (CDCl₃ 400 MHz) δ 8.05 (d, J=2.8 Hz, 1H), 7.00 (d, J=6.1 Hz, 1H), 4.19-4.08 (m, 2H), 1.46 (t, J=7.0 Hz, 3H).

Step 4: To a solution of compound G5-3 (640 g, 2.908 mmol) in toluene (6 L) was added n-BuLi (1.28 L, 3.20 mol, 1.1 eq. 2.5M) at −70° C. dropwise. The mixture was stirred at −70° C. for 0.5 h. To the mixture was added dimethylacetamide (431 g, 4.944 mol, 1.7 eq) at −65° C. dropwise and the reaction was allowed warm to 20° C. during a period of 30 mins. The reaction was quenched with aq.NH₄Cl (6 L). The aqueous layers was separated and extracted with ethyl acetate (4 L). The combined organic layers were washed with brine (1.5 L×2), dried and concentrated. The residue was purified by column chromatography (0˜10% of ethyl acetate in Hexane) to afford 1-(4-ethoxy-5-fluoropyridin-2-yl)ethan-1-one (G5-4) (330 g, 62% yield) as a pale yellow solid. ¹H NMR (CDCl₃ 400 MHz): δ 8.42-8.27 (m, 1H), 7.73-7.60 (m, 1H), 4.28-4.18 (m, 2H), 2.70-2.65 (m, 3H), 1.53-1.44 (m, 3H). LCMS: (MS (ESI) m/z 184.2 [M+H]⁺.

Step 5: To a solution of G5-4 (390 g, 2.13 mmol) in ethanol (4 L) was added NaBH₄ (96.7 g, 2.5 mol) at 10-20° C. in portions under N₂ atmosphere. The mixture was stirred at 25° C. for 1 h. The reaction was quenched with aq.NH₄Cl (900 mL) and water (6 L). The reaction was extracted with ethyl acetate (4 L×3). The combined organic layers were washed with brine (3 L), dried and concentrated. The residue was purified by column chromatography (10˜100% of ethyl acetate in Hexane) to afford 1-(4-ethoxy-5-fluoropyridin-2-yl)ethan-1-ol (G5-5) (340 g, 86% yield) as yellow oil. ¹H NMR (CD₃OD 400 MHz): δ 8.24-8.03 (m, 1H), 7.26 (dd, J=1.4, 7.2 Hz, 1H), 4.79 (q, J=6.5 Hz, 1H), 4.28-4.18 (m, 2H), 1.51-1.36 (m, 6H). LCMS: MS (ESI) m/z 186.2 [M+H]⁺.

Step 6: To a solution of G5-5 (250 g, 1.35 mmol) in dichloromethane (3 L) was added PPh₃ (425 g, 1.62 mol) and CBr₄ (537 g, 1.62 mol) at 0° C. in portions under N₂. The mixture was stirred at 25° C. for 1 h. The solution mixture was concentrated and purified by column chromatography (0˜20% of ethyl acetate in hexane) to 2-(1-bromoethyl)-4-ethoxy-5-fluoropyridine (G5) (320 g, 95.5% yield) as red oil. ¹H NMR (CDCl₃ 400 MHz): δ 8.26 (d, J=3.1 Hz, 1H), 7.04 (d, J=6.6 Hz, 1H), 5.16 (q, J=6.8 Hz, 1H), 4.20 (q, J=7.0 Hz, 2H), 2.04 (d, J=7.0 Hz, 3H), 1.50 (t, J=7.0 Hz, 3H). LCMS: MS (ESI) m/z 250.1 [M+H]⁺.

### Intermediate G6: 2-(bromomethyl)-4-ethoxy-5-fluoropyridine (G6)

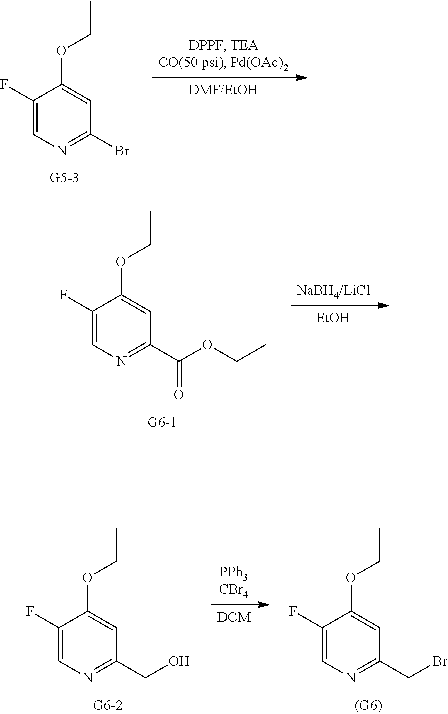

Step 1: To a solution of G5-3 (2 g, 9.09 mmol, 1.0 eq.) in DMF (30 mL) and ethanol (30 mL) was added Pd (OAc)₂ (204.3 mg, 0.91 mmol, 0.1 eq), DPPF (1 g, 1.82 mmol) and TEA (2.8 g, 27.3 mmol). The reaction was stirred at 50° C. for 16 hrs under CO (50 psi). The reaction mixture was diluted with water (200 mL) and extracted with ethyl acetate (50 mL×3). The combined organic layers were washed with brine (100 mL), dried and concentrated. The residue was purified by column chromatography (20˜30% of ethyl acetate in hexane) to give ethyl 4-ethoxy-5-fluoropicolinate (G6-1) (1.4 g, 72.2% yield) as white solid. ¹H NMR (CDCl₃ 400 MHz): δ 8.43 (d, J=2.7 Hz, 1H), 7.78 (d, J=7.0 Hz, 1H), 4.47 (q, J=7.1 Hz, 2H), 4.26 (q, J=7.0 Hz, 2H), 1.51 (t, J=7.0 Hz, 3H), 1.44 (t, J=7.2 Hz, 3H). LCMS: MS (ESI) m/z 214.0 [M+H]+

Step 2: To a solution of G6-1 (1.4 g, 5.57 mmol) in EtOH (28 mL) was added LiCl (1.18 g, 27.85 mmol) and NaBH₄ (1.05 g, 27.85 mmol) carefully at 0° C. under N₂. The solution was stirred at 20° C. for 3 hrs. Then the reaction was quenched by aq. NH₄Cl (100 mL) at 0° C. The mixture was extracted with ethyl acetate (50 mL×3). The combined organic layers were dried and concentrated to afford (4-ethoxy-5-fluoropyridin-2-yl)methanol (G6-2) (900 mg, 94.4% yield) as white solid. ¹H NMR (CDCl₃ 400 MHz): δ 8.25 (d, J=3.1 Hz, 1H), 6.88 (d, J=6.7 Hz, 1H), 4.68 (s, 2H), 4.18 (q, J=7.0 Hz, 2H), 1.49 (t, J=7.0 Hz, 3H).

Step 3: To a solution of G6-2 (200 mg, 1.17 mmol) in dichloromethane (20 mL) was added PBr₃ (507 mg, 1.872 mmol) at 0° C. under N₂. The mixture was stirred at 20° C. for 3 hrs. The mixture was quenched by 10% aqueous ammonia solution and adjust to pH 8. The mixture was diluted with H₂O (50 mL) and extracted with EA (30 mL×3). The combined organic layers were washed with H₂O (50 mL), dried and concentrated, then purified by column chromatography (0˜20% of ethyl acetate in hexane) to give 2-(bromomethyl)-4-ethoxy-5-fluoropyridine (G6) (240 mg, 86.3% yield) as yellow solid. ¹H NMR (CDCl₃ 400 MHz): δ 8.27 (d, J=3.2 Hz, 1H), 7.04 (d, J=6.6 Hz, 1H), 4.49 (s, 2H), 4.20 (q, J=7.0 Hz, 2H), 1.51 (t, J=7.0 Hz, 3H). LCMS: m/z 234.0 [M+H]⁺.

Intermediate G7: 5-(1-bromoethyl)-2-fluoro-3-methoxypyridine (G7)

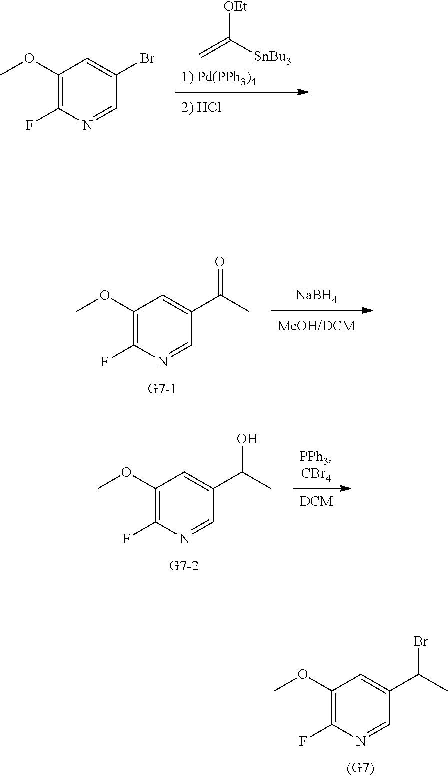

Step 1: To a solution of 5-bromo-2-fluoro-3-methoxypyridine (25 g, 121.35 mmol) in toluene (250.0 mL) was added Pd(PPh₃)₄ (9.82 g, 8.49 mmol) and tributyl(1-ethoxyvinyl)stannane (65.74 g, 182.03 mmol). The mixture was stirred at 100° C. for 16 hrs. The mixture was concentrated and to the residue was added aq.HCl (0.5 M, 200 mL) and THF (200 mL). The mixture was stirred at 15-20° C. for 1 hr. The mixture was adjusted to pH 8 by adding NaHCO₃. Then saturated KF (200 mL) was added. The solution was stirred at 15° C. for 1 hr. The mixture was extracted with EA (200 ml×3). The combined organic layers were dried concentrated and purified by column chromatography (9˜16% of ethyl acetate in Hexane) to 1-(6-fluoro-5-methoxypyridin-3-yl)ethan-1-one (G7-1) (16.7 g, 81.4% yield) as a white solid. ¹H NMR (CDCl₃, 400 MHz): δ ppm 8.33 (d, J=1.5 Hz, 1H), 7.83 (dd, J=1.6, 9.7 Hz, 1H), 3.96 (d, J=1.0 Hz, 3H), 2.63 (d, J=1.1 Hz, 3H). LCMS: MS (ESI) m/z 170.1 [M+H]⁺.

Step 2: To a solution of compound G7-1 (16.69 g, 98.67 mmol) in DCM (83 mL) and MeOH (83 mL) was added NaBH₄ (7.47 g, 197.34 mmol) at 0° C. in 3 portions. The mixture was stirred at 0° C. for 0.5 hr. The mixture was quenched with sat.NH₄Cl (100 ml) at 0° C., the aqueous phase was extracted with DCM (100 mL×2). The combined organic layers were dried and concentrated to afford 1-(6-fluoro-5-methoxypyridin-3-yl)ethan-1-ol (G7-2) (16.77 g, 99% yield) as a colorless oil. ¹H NMR (CDCl₃, 400 MHz): δ ppm 7.68-7.63 (m, 1H), 7.38 (dd, J=1.8, 9.8 Hz, 1H), 4.95 (q, J=5.8 Hz, 1H), 3.92 (s, 3H), 1.52 (d, J=6.4 Hz, 3H). LCMS: MS (ESI) m/z 172.2 [M+H]⁺.

Step 3: To a solution of compound G7-2 (16.77 g, 97 mmol) in DCM (120 mL) was added PPh₃ (38.5 g, 147 mmol) and then a solution of CBr₄ (48.75 g, 147 mmol) in DCM (40 mL) was added dropwise at 0° C. The mixture was stirred at 0° C. for 0.5 hr. The mixture was concentrated and the residue was purified by column chromatography (5˜9% of ethyl acetate in hexanes) to afford 5-(1-bromoethyl)-2-fluoro-3-methoxypyridine (G7) (16.7 g, 73% yield) as a white solid. ¹H NMR (CDCl₃ 400 MHz): δ ppm 7.74 (t, J=1.9 Hz, 1H), 7.38 (dd, J=2.1, 9.4 Hz, 1H), 5.18 (q, J=6.9 Hz, 1H), 3.94 (s, 3H), 2.06-2.04 (m, 3H). LCMS: MS (ESI) m/z 234.1 [M+H]⁺.

### Intermediate G8: 5-(bromomethyl)-2-fluoro-3-methoxypyridine (G8)

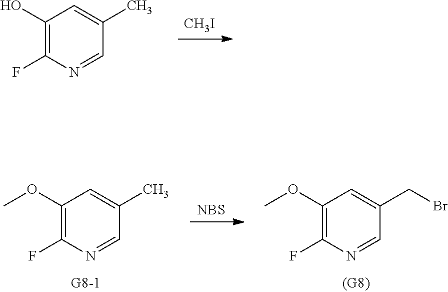

Step 1: To a reaction tube was added 2-fluoro-5-methylpyridin-3-ol (170 mg, 1.337 mmol), acetone (10 mL), K₂CO₃ (370 mg, 2.67 mmol) and iodomethane (0.167 mL, 2.67 mmol). The mixture was stirred at 80° C. for 16 hrs. and then filtered. The filtrate was concentrated and the residue was purified by flash column (20% EtOAc in hexane) to give 2-fluoro-3-methoxy-5-methylpyridine (G8-1) (150 mg, 79% yield) as white solid. LC-MS: MS m/z 142.1 [M+H]⁺.

Step 2: To a reaction tube was added compound G8-1 (153 mg, 1.084 mmol), CCl₄ (8 mL), AIBN (17.80 mg, 0.108 mmol) and 1-bromopyrrolidine-2,5-dione (212 mg, 1.192 mmol), stirred under N₂ at 80° C. for 16 h. Then solvent was removed, the residue was purified by flash column (20% EtOAc in hexane) to give 5-(bromomethyl)-2-fluoro-3-methoxypyridine (G8) (108 mg, 39.4% yield) as white solid. LC-MS: MS m/z 220.0 [M+H]⁺.

### Intermediate G9: 2-(1-bromoethyl)-5-fluoro-4-methoxypyridine (G9)

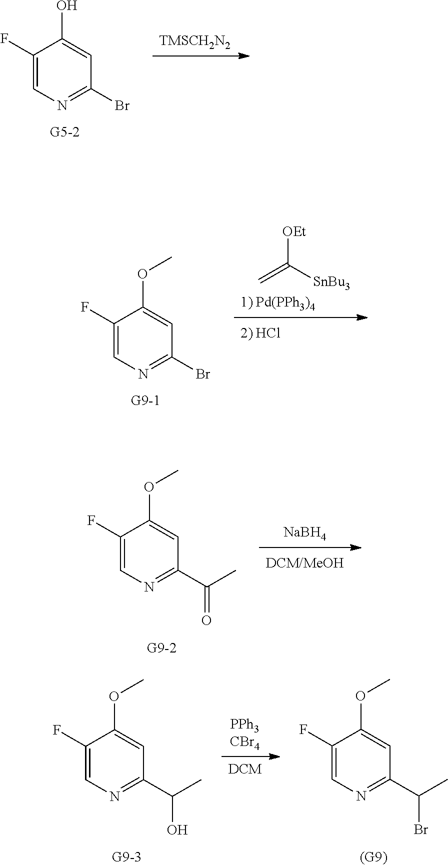

Step 1: To a solution of 2-bromo-5-fluoropyridin-4-ol (G5-2) (20 g, 104.17 mmol) in MeOH (25 mL) and CH₃CN (225 mL) was added DIEA (20.2 g, 156.26 mmol) and TMSCHN₂ (78 mL, 156.26 mmol, 2M in hexane) at 0° C. The mixture was stirred at 30° C. for 16 hrs. Water (200 mL) was added and the solution was concentrated to remove MeOH and CH₃CN. The aqueous layer was extracted with ethyl acetate (100 mL×3). The combined organic layers were washed with brine (200 mL), dried and concentrated, the residue was purified by column chromatography (1˜25% of ethyl acetate in Hexane) to afford 2-bromo-5-fluoro-4-methoxypyridine (G9-1) (13.8 g, 64% yield) as colorless oil. ¹H NMR (CDCl₃ 400 MHz) δ8.10 (d, J=2.6 Hz, 1H), 7.05 (d, J=6.1 Hz, 1H), 4.04-3.86 (m, 3H).

Step 2: To a solution of compound G9-1 (13.8 g, 66.99 mmol) in toluene (170 mL) was added tributyl(1-ethoxyvinyl)stannane (29.03 g, 80.38 mmol) and Pd (PPh₃)₄ (3.87 g, 3.35 mmol) under nitrogen atmosphere. The mixture was stirred at 110° C. for 18 hours. The solution was cooled to 25° C. To the mixture was added HCl (130 mL, 1M) and THF (1300 mL) and the mixture was stirred at 25° C. for 2 hours. To the resulting mixture was added sat Na₂CO₃ solution (140 mL) and the pH adjusted to pH 8˜9. Then aq. KF (200 mL) was added to the mixture and the mixture was stirred for 1 h. The mixture was filtered and the filtrate was extracted with ethyl acetate (400 mL×2). The combined organic layers were dried and concentrated, then purified by column chromatography (1˜25% of ethyl acetate in hexane) to 1-(5-fluoro-4-methoxypyridin-2-yl)ethan-1-one (G9-2) (13 g, crude) as a yellow oil. ¹H NMR (CDCl₃ 400 MHz) δ 8.37 (d, J=2.7 Hz, 1H), 7.72 (d, J=7.2 Hz, 1H), 4.00 (s, 3H), 2.75-2.66 (m, 1H), 2.75-2.66 (m, 1H), 2.70 (s, 3H).

Step 3: To a solution of compound G9-2 (12.95 g, 76.56 mmol) in EtOH (260 mL) was added NaBH₄ (5.79 g, 153.12 mmol) at 0° C. under N₂. The reaction was stirred at 0° C. for 2 hour. The reaction was quenched by aq. NH₄Cl and concentrated, diluted with water (100 ml) extracted with ethyl acetate (400 mL×3). The combined organic layers were washed with brine (800 mL), dried and concentrated. The crude was purified by column chromatography (5˜100% of ethyl acetate in Hexane) to 1-(5-fluoro-4-methoxypyridin-2-yl)ethan-1-ol (G9-3) (6.97 g, 53% yield) as colorless oil. ¹H NMR (CDCl₃ 400 MHz) δ 8.23 (d, J=2.9 Hz, 1H), 6.90 (d, J=6.8 Hz, 1H), 4.83 (q, J=6.4 Hz, 1H), 3.95 (s, 3H), 1.48 (d, J=6.5 Hz, 3H).

Step 4: To a solution of compound G9-3 (6.5 g, 37.97 mmol, 1.0 eq.) and PPh₃ (14.94 g, 56.96 mmol) in dichloromethane (200 mL) was added a solution of CBr₄ (18.89 g, 56.96 mmol) in dichloromethane (60 mL) at 0° C. The mixture was stirred at 25° C. for 5 hours. The mixture was concentrated. The residue was purified by column chromatography (1˜20% of ethyl acetate in hexane) to afford 2-(1-bromoethyl)-5-fluoro-4-methoxypyridine (G9) (5.00 g, 56% yield) as colorless oil. ¹H NMR (CDCl₃ 400 MHz) δ 8.26 (d, J=2.9 Hz, 1H), 7.06 (d, J=6.7 Hz, 1H), 5.17 (q, J=6.9 Hz, 1H), 3.97 (s, 3H), 2.05 (d, J=6.9 Hz, 3H).

### Intermediate G10: 2-(bromomethyl)-5-fluoro-4-methoxypyridine (G10)

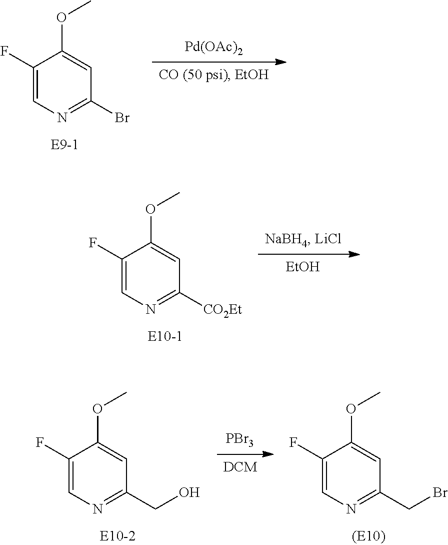

Step 1: A mixture of 2-bromo-5-fluoro-4-methoxypyridine (G9-1) (300 mg, 1.5 mmol), Pd(OAc)₂ (34 mg, 0.15 mmol), DPPP (250 mg, 0.45 mmol) and NaOAc (615 mg, 7.5 mmol) in ethanol (20 mL) was stirred at 70° C. under CO (50 psi) for 48 hrs. The mixture was concentrated and the crude was dissolved in the mixed solvent of dichloromethane/ethyl acetate (20 mL/3 mL). The mixture was filtered and the filtrate was concentrated and purified by prep-HPLC (Column: Boston Green ODS 150*30 5 u, gradient: 12-42% of B, A=water (0.1% TFA), B=acetonitrile, flow rate: 30 mL/min) to ethyl 5-fluoro-4-methoxypicolinate (G10-1) (180 mg, 62% yield) as a yellow solid. ¹H NMR (CD₃OD 400 MHz) δ 8.42 (d, J=2.8 Hz, 1H), 7.92 (d, J=2.8 Hz, 1H), 4.45 (q, J=7.2, Hz, 2H), 4.07 (s, 3H), 1.43 (t, J=7.2 Hz, 3H). LCMS: MS (ESI) m/z 200.0 [M+H]⁺.

Step 2: To a solution of compound G10-1 (180 mg, 0.9 mmol) and LiCl (124 mg, 2.7 mmol) in ethanol (5 mL) was added NaBH₄ (103 mg, 2.7 mmol). The mixture was stirred at 25° C. for 18 hours. The mixture was quenched with saturated NH₄Cl (5 mL) and extracted with ethyl acetate (20 mL×2). The combined organic layers were washed with brine (10 mL), dried and concentrated, then purified by flash column (0˜60% ethyl acetate in hexane) to afford (5-fluoro-4-methoxypyridin-2-yl)methanol (G10-2) (80 mg, 56% yield) as a white solid. ¹H NMR (CDCl₃ 400 MHz) δ 8.28 (d, J=2.8 Hz, 1H), 6.88 (d, J=2.8 Hz, 1H), 4.71 (s, 2H), 3.96 (s, 3H).

Step 3: A solution of compound G10-2 (20 mg, 0.127 mmol) in dichloromethane (5 mL) was added PBr₃ (55 mg, 0.2 mmol) at 0° C. under N₂. The mixture was stirred at 40° C. for 2 hrs. The mixture was cooled to 0° C. and NH₄OH (0.1 mL) was added. The solution was extracted with dichloromethane (10 mL×2). The combined organic layers were dried and concentrated at 5° C. to give 2-(bromomethyl)-5-fluoro-4-methoxypyridine (G10) (27 mg, crude). LCMS: MS (ESI) m/z 220.0 [M+H]⁺.

### Intermediate G11: 2-(1-bromoethyl)-5-chloro-4-ethoxypyridine (G11)

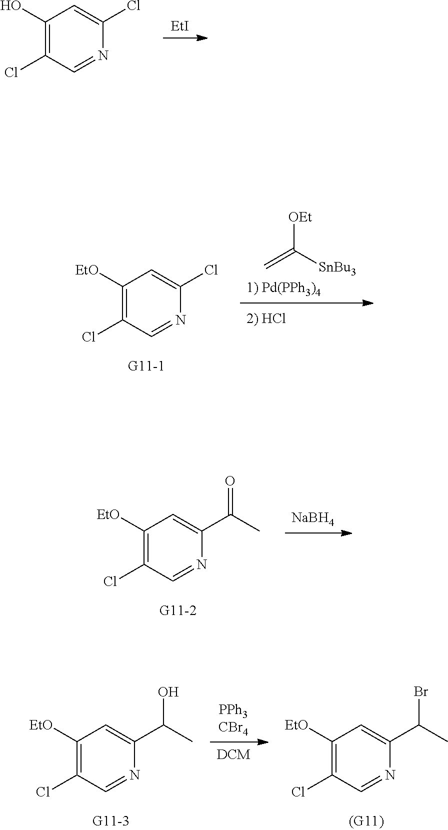

Step 1: To a reaction tube was added 2,5-dichloropyridin-4-ol (410 mg, 2.5 mmol), K₂CO₃ (691 mg, 5.00 mmol), acetone (10 mL) and iodoethane (0.402 mL, 5.00 mmol), and the mixture was heated to 80° C. and stirred for 18 hrs. The mixture was then filtered and the filtrate was concentrated and purified by flash column (30% EtOAc in hexane) to give 2,5-dichloro-4-ethoxypyridine (G11-1) (340 mg, 70.8% yield) as white solid. LC-MS: MS m/z 191.9 [M+H]⁺.

Step 2: To a reaction tube was added compound G11-1 (200 mg, 1.041 mmol) and Pd(PPh₃)₂Cl₂ (110 mg, 0.156 mmol). Under N, 1,4-Dioxane (8 mL) and 1,4-Dioxane (8 mL) were added, and the mixture then stirred at 95° C. for 16 hrs. Then HCl (1.0 M, 4.0 mL) was added and the mixture was stirred at rt for a further 3 hrs. The reaction was quenched with Sat. NaHCO₃ (aq.), extracted with EtoAc, dried and concentrated, the residue was purified by flash column (30% EtOAc in hexane) to give 1-(5-chloro-4-ethoxypyridin-2-yl)ethan-1-one (G11-2) (150 mg, 61% yield). LC-MS: MS m/z 200.0[M+H]⁺.

Step 3: To a flask was added compound G11-2 (150 mg, 0.639 mmol) and ethanol (6 mL). At 0° C., NaBH₄ (48.3 mg, 1.277 mmol) was added, then the mixture was stirred at rt for 30 min. The reaction was slowly quenched with NH₄Cl (aq), extracted with EtOAc, dried and concentrated. The residue was purified by flash column (50%-100% EtOAc in hexane) to give 1-(5-chloro-4-ethoxypyridin-2-yl)ethan-1-ol (G11-3) (96 mg, 74.5% yield). ¹H NMR (400 MHz, Methanol-d₄) δ 6.80 (s, 1H), 5.75 (s, 1H), 3.32 (q, J=6.6 Hz, 1H), 2.78 (q, J=7.0 Hz, 2H), 0.03-−0.06 (m, 6H). LC-MS: MS m/z 202.0 [M+H]⁺.

Step 4: Compound (G11-3) (70 mg, 0.347 mmol), CH₂Cl₂ (5 mL), and PPh₃ (137 mg, 0.521 mmol) were added to a reaction tube then, at 0° C., perbromomethane (173 mg, 0.521 mmol) in DCM (1 mL) was added and the mixture was stirred at rt for 3 hrs. The solvent was evaporated and the residue was purified by flash column (30% EtOAc in hexane) to give 2-(1-bromoethyl)-5-chloro-4-ethoxypyridine (G11) (60 mg, 65.3% yield) as colorless oil. LC-MS: MS m/z 265.9 [M+H]⁺.

### Intermediate G12: 6-(1-bromoethyl)-4-methoxynicotinonitrile (G12)

### 6-(1-bromoethyl)-4-methoxynicotinonitrile

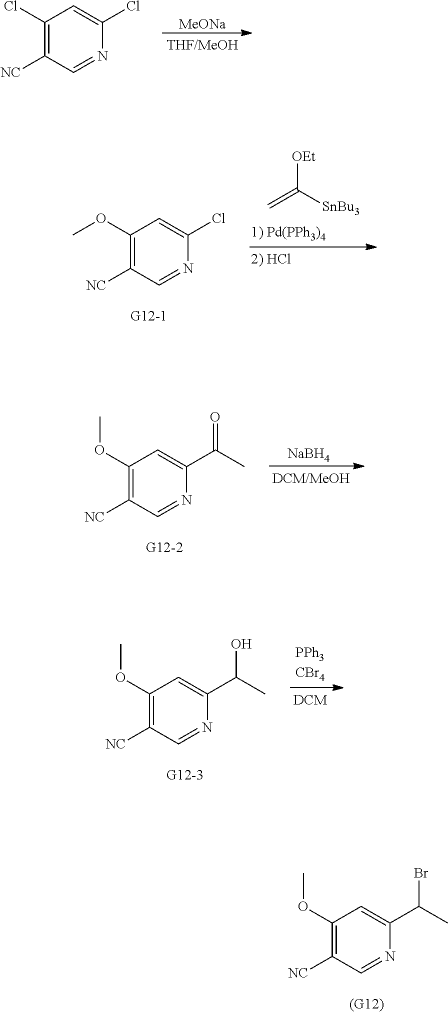

Step 1: To a solution of 4,6-dichloronicotinonitrile (30 g, 173.4 mmol) in THF (750 mL) MeONa (6.24 g, 115.6 mmol) in MeOH (40 mL) was added dropwise while the solution was stirred in an ice/water bath. Then the mixture was stirred at 0° C. for 1 hr. The mixture was filtered and the filtrate was concentrated and purified by column chromatography (2˜10% of ethyl acetate in Hexane) to afford 6-chloro-4-methoxynicotinonitrile (G12-1) as a white solid.

LCMS: MS (ESI) m/z 169.1 [M+H]⁺.

Step 2: To a solution of compound G12-1 (5 g, 29.66 mmol) in toluene (100 mL) was added tributyl(1-ethoxyvinyl)stannane (12.85 g, 35.59 mmol, 1.2 eq), and Pd(PPh₃)₄ (1.713 g, 1.483 mmol). The reaction mixture was stirred for 4 hrs at 110° C. under N₂. The reaction mixture was concentrated. To the residue was added THF (120 mL) followed by 2M HCl (120 mL). The mixture was stirred for 2 hrs at 25° C. before being filtered. The filtrate was extracted with EA (100 mL×3). The combined organic layers were washed with brine (300 mL), dried and concentrated. The residue was purified by column chromatography (10˜17% of ethyl acetate in Hexane) to give 6-acetyl-4-methoxynicotinonitrile (G12-2) (4.2 g, 23.84 mmol, 80.4%) as a light yellow solid. LCMS: MS (ESI) m/z 177.1 [M+H]⁺.

Step 3: To a solution of compound G12-2 (2.5 g, 14.2 mmol, 1.0 eq) in DCM/MeOH (50 mL/50 mL) was added NaBH4 (1.01 mg, 28.4 mmol, 2.0 eq) at −78° C. The mixture was slowly warm to 0° C. and stirred at 0° C. for 0.5 hr. The mixture was filtered. The filtrate was concentrated and purified by column chromatography (10-50% of ethyl acetate in Hexane) on silica gel to afford 6-(1-hydroxyethyl)-4-methoxynicotinonitrile (G12-3) (2.2 g, 86% yield) as a light yellow solid. LCMS: MS (ESI) m/z 179.2 [M+H]⁺.

Step 4: To a solution of compound G12-3 (2 g, 11.22 mmol, 1.0 eq) in dichloromethane (250 mL) were added CBr₄ (5.58 g, 16.8 mmol, 1.5 eq) and triphenylphosphine (4.41 g, 16.83 mmol, 1.5 eq). The mixture was then stirred at 25° C. for 16 hrs. The mixture was concentrated and purified by column chromatography (15˜30% of ethyl acetate in Hexane) on silica gel to afford 6-(1-bromoethyl)-4-methoxynicotinonitrile (G12) (1.7 g, 62.8% yield) as a yellow oil. LCMS: MS (ESI) m/z 241.1 [M+H]⁺.

### Intermediate G13: 4-chloro-1-(difluoromethyl)-5-iodopyridin-2(1H)-one (G13)

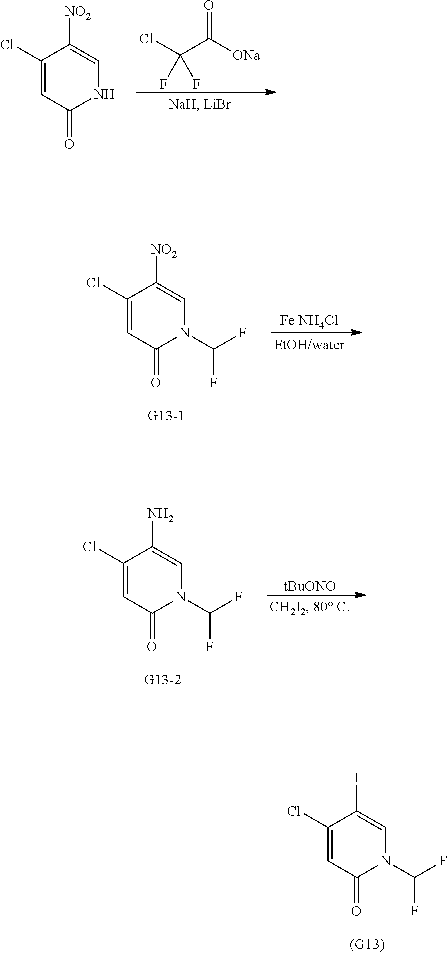

Step 1: To a solution of 4-chloro-5-nitropyridin-2(1H)-one (3.5 g, 20.1 mmol, 1 equiv) in DMF (30 mL) was added NaH (1.13 g, 28.2 mmol, 1.4 equiv, 60% in oil) at 0° C. under nitrogen atmosphere. The reaction mixture was stirred at 0° C. for 15 min., then LiBr (3.43 g, 40.2 mmol, 2 equiv and sodium 2-chloro-2,2-difluoroacetate (6.11 g, 40.2 mmol, 2 equiv) were added to the mixture at 0° C. and the reaction mixture was stirred at 0° C. for 5 min. The mixture was then stirred at 120° C. for 1.5 hours and then cooled to 25° C. Water (20 mL) was added to the mixture and the resulting mixture was filtered and extracted with ethyl acetate (30 mL×3). The combined organic phase was concentrated under reduced pressure. The residue was purified by silica gel chromatography (petroleum ether/ethyl acetate=5:1) to afford 4-chloro-1-(difluoromethyl)-5-nitropyridin-2(1H)-one (G13-1) (1.1 g, 4.9 mmol, 24% yield) as yellow solid. ¹H NMR (400 MHz, CD₃OD) δ 8.93 (s, 1H), 7.90-7.59 (m, 1H), 6.86 (s, 1H).

Step 2: To a solution of compound G13-1 (600 mg, 2.68 mmol, 1 equiv) in EtOH (16 mL) and H₂O (4 mL) was added Fe (750 mg, 13.39 mmol, 5 equiv) and NH₄Cl (143 mg, 2.68 mmol, 1 equiv) at 25° C. The reaction mixture was stirred at 80° C. for 2 hours. The reaction mixture was filtered and washed with MeOH (5 mL×3) and the filtrate then concentrated under reduced pressure. The residue was purified by silica gel chromatography (petroleum ether/ethyl acetate=1:1) to afford compound 5-amino-4-chloro-1-(difluoromethyl)pyridin-2(1H)-one (G13-2) (520 mg, 2.69 mmol) as yellow solid. LCMS: 195.1 [M+H]⁺

Step 3: To a solution of compound G13-2 (250 mg, 1.29 mmol, 1 equiv) in CH₂I₂ (5 mL) was added tBuONO (199 mg, 1.93 mmol, 1.5 equiv) slowly at 0° C. while stirring vigorously. The ice bath was removed and the reaction mixture was stirred at 25° C. for 10 min. The mixture was then stirred at 80° C. for 3 hours. TLC (petroleum ether/ethyl acetate=1:1) showed reaction completion and the reaction mixture was then purified by silica gel chromatography directly (petroleum ether/ethyl acetate=5:1) to afford 4-chloro-1-(difluoromethyl)-5-iodopyridin-2(1H)-one (G13) (52 mg, 65% purity) as yellow solid. LCMS: 305.9 [M+H]+.

Intermediate G14: 2-(4-bromo-5-methylpyridin-2-yl)propan-2-ol (G14)

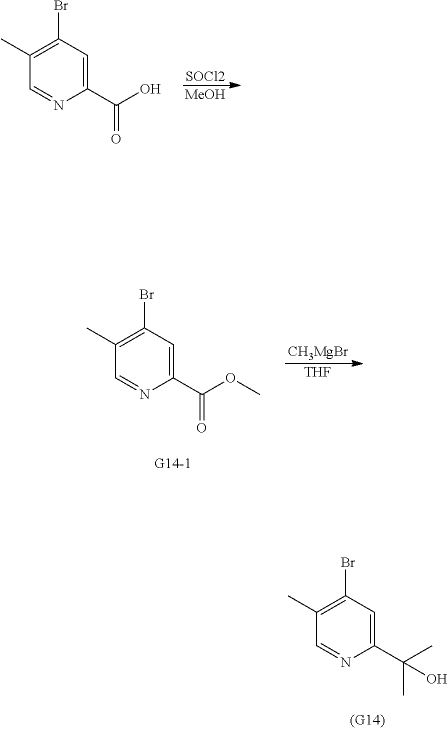

Step 1: To a solution of 4-bromo-5-methylpicolinic acid (400 mg, 1.85 mmol, 1.0 eq) in MeOH (2 mL) was added SOCl₂ (1.76 g, 14.8 mmol, 8.0 eq) at 0° C. The reaction was stirred at 25° C. for 16 hrs. The mixture was diluted with H₂O (5 mL) and quenched with aq.NaHCO₃ solution to pH 8. The resulting mixture was extracted with acetic ether (10 mL×2). The combined organic layers were dried over anhydrous Na₂SO₄, filtered and concentrated in vacuum to give methyl 4-bromo-5-methylpicolinate (G14-1) (160 mg, 34.1% yield, 90.7% purity) as yellow oil. ¹H NMR (400 MHz, CDCl₃) δ 8.56 (s, 1H), 8.53 (s, 1H), 8.31 (s, 1H), 8.12 (s, 1H), 4.01 (s, 5H), 2.46 (s, 3H), 2.45 (s, 1H).

Step 2: To a solution of compound G14-1 (160 mg, 0.695 mmol, 1.0 eq) in THF (3 mL) was added CH₃MgBr (0.6 mL, 1.738 mmol, 2.5 eq, 3M in diethyl ether) at 0° C. under N₂. The reaction was stirred at 0° C. for 30 mins. The mixture was then stirred at 25° C. for 16 hrs. TLC (petroleum ether:acetic ether=2:1) showed compound G14-1 (Rf=0.7) was consumed and a new spot (Rf=0.6) formed. The mixture was quenched with aq.NH₄Cl solution (10 mL). The resulting mixture was extracted with EtOAc (10 mL×3). The combined organic layers were dried over anhydrous Na₂SO₄, filtered and concentrated in vacuum. The residue was purified by column chromatography on silica gel eluting with petroleum ether: EtOAc (30%˜50% EtOAc) to give 2-(4-bromo-5-methylpyridin-2-yl)propan-2-ol (G14) (40 mg crude) as yellow oil. LCMS: 229.9/231.9.

### Intermediate G15: 4-bromo-1-methyl-3-(trifluoromethyl)pyridin-2(1H)-one (G15)

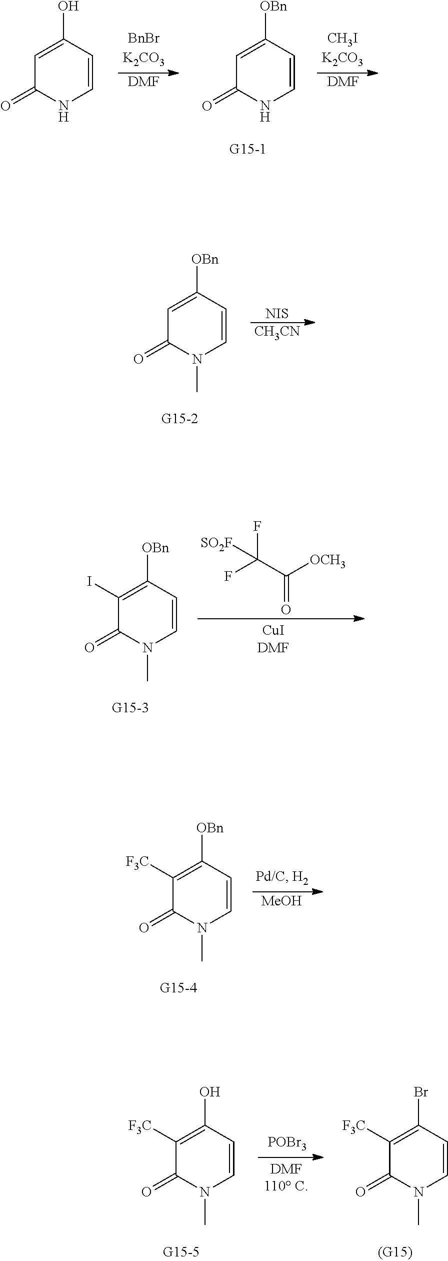

Step 1: To a suspension 4-hydroxypyridin-2(1H)-one (20 g, 0.18 mol, 1.0 eq.) and K₂CO₃ (49.7 g, 0.36 mol, 2.0 eq.) in DMF (300 mL) was added BnBr (32.3 g, 0.189 mol, 1.05 eq) at 0° C. The mixture was stirred at 25° C. for 1 hr. TLC (dichlormethane/methanol=10/1) showed near consumption of compound starting material was and formation of a new spot. The reaction mixture was diluted with water/brine (400/400 mL) and extracted with ethyl acetate (500 mL×3). The combined organic layers were washed with brine (300 mL×2), dried over anhydrous Na₂SO₄, filtered and concentrated. Petroleum ether/ethyl acetate (50/10 mL) was added to the residue and the mixture was stirred for 1 hr. The mixture was filtered and the solid was dried in vacuum to afford 4-(benzyloxy)pyridin-2(1H)-one (G15-1) (6.7 g, 18.5% yield) as a white solid. ¹H NMR (400 MHz, DMSO): δ 11.14 (br s, 1H), 7.48-7.30 (m, 5H), 7.25 (d, J=7.3 Hz, 1H), 5.92 (dd, J=2.4, 7.3 Hz, 1H), 5.80 (d, J=2.6 Hz, 1H), 5.06 (s, 2H)

Step 2: To a suspension of compound G15-1 (6.7 g, 33.30 mmol, 1.0 eq.) and K₂CO₃ (13.8 g, 99.89 mmol, 3.0 eq.) in DMF (80 mL) was added CH₃I (9.45 g, 66.59 mmol, 2.0 eq). The mixture was stirred at 25° C. for 2 hrs. TLC (100% ethyl acetate) showed compound G15-1 was consumed. The reaction mixture was diluted with water/brine (100/100 mL) and extracted with ethyl acetate (200 mL×3). The combined organic layers were washed with brine (100 mL×2), dried over anhydrous Na₂SO₄, filtered and concentrated. The residue was purified by Biotage-Flash (gradient: 0-100% of ethyl acetate, A=petroleum ether, B=ethyl acetate) to afford 4-(benzyloxy)-1-methylpyridin-2(1H)-one (G15-2) (4 g, 55.8% yield) as a white solid. ¹H NMR (400 MHz, DMSO): δ 7.56 (d, J=7.6 Hz, 1H), 7.44-7.31 (m, 5H), 5.98 (dd, J=2.8, 7.5 Hz, 1H), 5.88 (d, J=2.8 Hz, 1H), 5.06 (s, 2H), 3.32 (s, 3H).

Step 3: To a solution of compound G15-2 (4 g, 18.58 mmol, 1.0 eq.) in CH₃CN (80 mL) was added NIS (7.5 g, 33.45 mmol, 1.8 eq). The mixture was stirred at 25° C. for 4 hrs and then the mixture was concentrated. The residue was purified by Biotage-Flash (gradient: 0-80% of ethyl acetate, A=petroleum ether, B=ethyl acetate) to afford 4-(benzyloxy)-3-iodo-1-methylpyridin-2(1H)-one (G15-3) (4 g, 63.1% yield) as a white solid. LCMS: 342.0 [M+H]+

Step 4: To a solution of compound G15-3 (4 g, 11.72 mmol, 1.0 eq.) and methyl 2,2-difluoro-2-((fluorosulfinyl)oxy)acetate (4.5 g, 23.45 mmol, 2.0 eq) in DMF (80 mL) was added CuI (4.5 g, 23.45 mmol, 2.0 eq). The mixture was stirred at 100° C. for 4 hrs. The reaction was filtered and concentrated and the residue purified by Biotage-Flash (gradient: 0-80% of ethyl acetate, A=petroleum ether, B=ethyl acetate) to afford 4-(benzyloxy)-1-methyl-3-(trifluoromethyl)pyridin-2(1H)-one (G15-4) (3.2 g, 96.3% yield) as a white solid. ¹H NMR (400 MHz, CD₃OD): δ 7.79 (d, J=7.8 Hz, 1H), 7.43-7.29 (m, 5H), 6.41 (d, J=7.8 Hz, 1H), 5.27 (s, 2H), 3.52-3.41 (m, 3H)

Step 5: To a solution of compound G15-4 (1.6 g, 5.645 mmol, 1.0 eq.) in ethanol (30 mL) was added Pd/C (1.6 g, 10% Pd, 50% water). The mixture was stirred at 25° C. for 3 hrs under H₂ (15 psi) atmosphere. The reaction was filtered and concentrated to afford 4-hydroxy-1-methyl-3-(trifluoromethyl)pyridin-2(1H)-one (G15-5) (1.1 g, crude) as white solid. LCMS: 194.1 [M+H]+

Step 6: To a solution of compound G15-5 (500 mg, 2.59 mmol, 1.0 eq.) in DMF (10 mL) was added POBr₃ (1.86 g, 6.47 mmol, 2.5 eq). The mixture was stirred at 110° C. for 2 hrs. The reaction was quenched with ice water/brine (10 mL/10 mL) and extracted with ethyl acetate (20 mL×3). The combined organic layers were washed with brine (20 mL×2), dried over anhydrous Na₂SO₄, filtered and concentrated. The residue was purified by Biotage-Flash (gradient: 0-50% of ethyl acetate, A=petroleum ether, B=ethyl acetate) to afford 4-bromo-1-methyl-3-(trifluoromethyl)pyridin-2(1H)-one (G15) (350 mg, 53% yield) as oil. ¹H NMR (400 MHz, DMSO): δ 7.93 (d, J=7.2 Hz, 1H), 6.66 (d, J=7.2 Hz, 1H), 3.45 (s, 3H), 1.89-1.86 (m, 1H)

### Intermediate G16: (2R,3R)-2-methylazetidin-3-ol (G16)

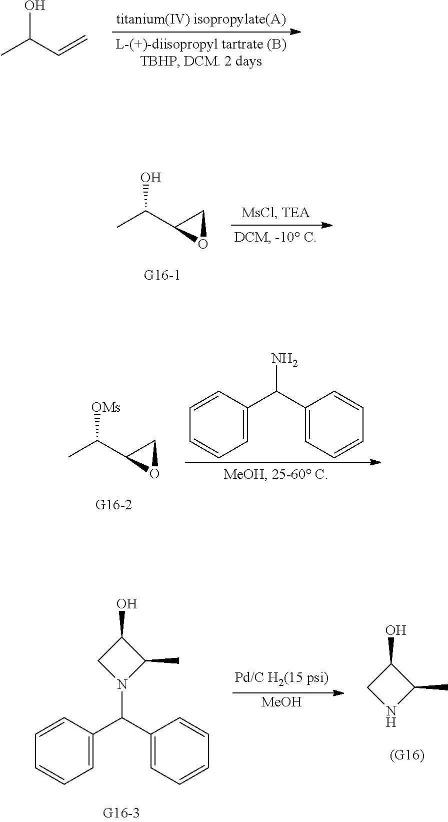

Step 1. To a suspension of 4 A molecular sieves (7.14 g) in dichloromethane (500 mL) were added but-3-en-2-ol (15 mL, 173 mmol, 1.0 eq) and L-(+)-diisopropyl tartrate (5.6 mL, 26 mmol, 0.15 eq) at 25° C. under nitrogen atmosphere. The reaction was cooled to −20° C. and titanium (IV) isopropylate (5.14 mL, 17.3 mmol, 0.1 eq) was added. The resulting mixture was stirred at −20° C. for 1 hr. TBHP (14.14 mL, 77.78 mmol, 0.45 eq) was added dropwise at −20° C. and the resulting mixture was stirred at −20° C. for 40 hrs. The reaction was quenched with water/acetone (8/16 mL). The mixture was filtered and the filtrate was concentrated. The crude product was purified by distillation (58-62° C., water bump) to afford (S)-1-((R)-oxiran-2-yl)ethan-1-ol (G16-1) (4.5 g, 31% yield) as colorless oil. ¹H NMR (CDCl₃ 400 MHz): δ 4.00 (dq, J=3.1, 6.3 Hz, 1H), 3.07-2.97 (m, 1H), 2.83-2.77 (m, 1H), 2.76-2.71 (m, 1H), 2.12-1.88 (m, 1H), 1.28-1.23 (m, 6H).

Step 2: To a solution of compound G16-1 (4.5 g, 51.1 mmol, 1.0 eq.) and triethylamine (15.5 g, 1532.2 mmol, 3.0 eq) in dichloromethane (90 mL) was added methanesulfonyl chloride (11.7 g, 102.1 mmol, 2.0 eq) at −20˜−10° C. under nitrogen atmosphere. The reaction was stirred at −10° C. for 1 hr. TLC (petroleum ether/ethyl acetate=2/1) showed compound G16-1 was consumed. The reaction was diluted with water (100 mL) and extracted with dichloromethane (100 mL×2). The combined organic layers were washed with brine (100 mL), dried over sodium sulfate, filtered and concentrated. The residue was purified by Biotage-Flash (gradient: 0˜60% of ethyl acetate, A=petroleum ether, B=ethyl acetate) to afford (S)-1-((R)-oxiran-2-yl)ethyl methanesulfonate (G16-2) (5 g, 59% yield) as colorless oil. ¹H NMR (CDCl₃ 400 MHz): δ 4.72 (dq, J=4.2, 6.5 Hz, 1H), 3.08 (dt, J=2.6, 4.0 Hz, 1H), 3.02 (s, 3H), 2.83-2.80 (m, 1H), 2.85-2.78 (m, 1H), 2.79-2.73 (m, 1H), 1.44 (d, J=6.5 Hz, 3H).

Step 3: To a solution of compound G16-2 (5 g, 30.08 mol, 1.0 eq) and benzhydrylamine (6.6 g, 36.10 mmol, 1.2 eq) in methanol (50 mL) was stirred at 25° C. for 4 h. The reaction was heated to 60° C. and stirred for 18 h. The reaction solution was concentrated. The residue was purified by Prep-HPLC (Column: kromasil Eterning XT 250*80*10 um; Condition: A=water (0.05% ammonia hydroxide)-acetonitrile; 30-55% of acetonitrile, flow rate: 160 ml/min) to (2R,3R)-1-benzhydryl-2-methylazetidin-3-ol (G16-3) (3.5 g, 46% yield) as off-white solid. ¹H NMR (400 MHz, CDCl₃): δ 7.32 (dd, J=7.7, 16.3 Hz, 4H), 7.22-7.06 (m, 6H), 4.29 (s, 1H), 4.21 (br t, J=5.3 Hz, 1H), 3.40-3.31 (m, 1H), 3.18 (br d, J=9.1 Hz, 1H), 3.00-2.94 (m, 1H), 2.93-2.68 (m, 1H), 0.66 (dd, J=2.4, 6.5 Hz, 3H).

Step 4: To a mixture of compound G16-3 (2 g, 7.89 mmol 1.0 eq) and Pd/C (1 g, 10% Pd, 50% water) in MeOH (40 mL) was stirred at 25° C. for 30 hrs under H₂ (15 psi) atmosphere. TLC (petroleum ether/ethyl acetate=1/1) showed compound G16-3 was consumed. The mixture was filtered and the filtrate was concentrated to (2R,3R)-2-methylazetidin-3-ol (G16) (2 g, crude) as oil.

### Intermediate G17: (2R,3R)-2-methylpyrrolidin-3-ol (G17)

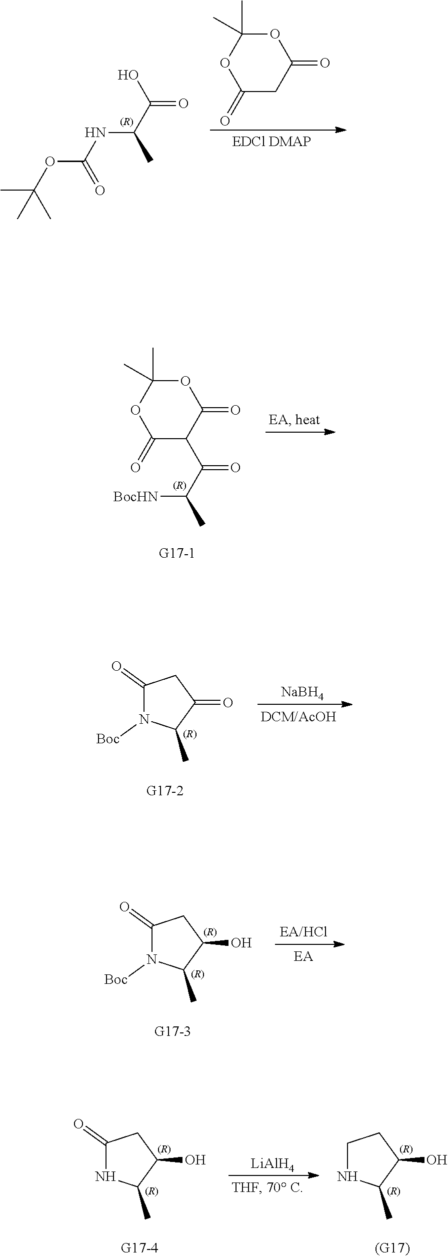

Step 1: To a stirred solution of 2,2-dimethyl-1,3-dioxane-4,6-dione (16.77 g, 116 mmol, 1.1 equiv) and DMAP (19.32 g, 15.8 mmol, 1.5 equiv) in dichloromethane (500 mL) was added (tert-butoxycarbonyl)-D-alanine (20 g, 106 mmol, 1 equiv) in one portion at 0° C. Then EDCI (48.7 g, 253.8 mmol, 2.4 equiv) was added in one portion at 0° C. The reaction mixture was then stirred at 25° C. for 12 hours. The reaction mixture was concentrated under reduced pressure at 30° C., and then diluted with ethyl acetate (300 mL). The resulting solution was washed with 5% KHSO₄ (100 mL×3) and brine (75 mL). The organic layer was dried over Na₂SO₄ and filtered to afford a solution of tert-butyl (R)-(1-(2,2-dimethyl-4,6-dioxo-1,3-dioxan-5-yl)-1-oxopropan-2-yl)carbamate (G17-1) in ethyl acetate (200 mL), which was used for the next step directly. LCMS: 216.0 [M+H-100]+

Step 2: Compound G17-1 (a solution in ethyl acetate (200 mL) from step 1) was stirred at 90° C. for 4 hours under nitrogen atmosphere. The reaction mixture was concentrated under reduced pressure to afford tert-butyl (R)-2-methyl-3,5-dioxopyrrolidine-1-carboxylate (G17-2) (16 g, crude) as yellow solid, which was used for the next step directly.

Step 3: To a solution of compound G17-2 (1.5 g, 7.03 mmol, 1 eq, crude) in DCM (70 mL) was added AcOH (5 mL) at 0° C. under nitrogen atmosphere. Then NaBH₄ (532 mg, 14.7 mmol, 2 eq) was added slowly in portions at 0° C. under nitrogen atmosphere. The reaction mixture was stirred at 0° C. for 3 hours. TLC (dichloromethane/Methanol=10:1) showed formation of new compound. The reaction mixture was adjust pH=8 by sat NaHCO₃. The resulting mixture was extracted with ethyl acetate (20 mL×3). The combined organic phases were concentrated under reduced pressure. The residue was purified by silica gel chromatography (petroleum ether/ethyl acetate=1:1) to afford tert-butyl (2R,3R)-3-hydroxy-2-methyl-5-oxopyrrolidine-1-carboxylate (G17-3) (800 mg, 3.7 mmol, 53% yield) as white solid. ¹H NMR (400 MHz, CDCl₃): δ 4.50-4.36 (m, 1H), 4.18 (q, J=6.4 Hz, 1H), 2.69-2.59 (m, 1H), 2.56-2.44 (m, 1H), 2.13-2.02 (m, 1H), 1.51-1.42 (m, 9H), 1.26 (d, J=6.4 Hz, 3H).

Step 4: To a solution of compound G17-3 (400 mg, 1.86 mmol, 1 equiv) in ethyl acetate (8 mL) was added HCl/ethyl acetate (6 mL, 4M) at 25° C. The reaction mixture was stirred at 25° C. for 1 hr. The reaction mixture was concentrated under reduced pressure to afford (4R,5R)-4-hydroxy-5-methylpyrrolidin-2-one (G17-4) (500 mg, crude) as yellow solid. LCMS: 116.06 [M+H]+

Step 5: To a solution of compound (G17-4) (500 mg, 1.86 mmol, 1 equiv, crude) in THF (10 mL) was added LiAlH₄ (212 mg, 5.58 mmol, 3 equiv) at 25° C. for 10 min. Then the reaction mixture was stirred at 70° C. for 8 hours. TLC (dichloromethane/Methanol=10:1) showed complete reaction. The reaction mixture was cooled to 25° C. and H₂O (1 mL) was added while stirring quickly. Then 1 M NaOH (1 mL) and H₂O (3 mL) was added. The resulting mixture was stirred at 25° C. for 10 min and then filtered. The filter cake was washed with THF (5 mL×3). The combined organic phase was concentrated under reduced pressure. The residue was dissolved in MeOH (1 mL) and dichloromethane (20 mL). The resulting mixture was dried over Na₂SO₄ and filtered. The organic phase was concentrated under reduced pressure to afford (2R,3R)-2-methylpyrrolidin-3-ol (G17) (150 mg, crude) as a yellow oil. ¹H NMR (400 MHz, CDCl₃): δ=4.00 (m, 1H), 3.64-3.57 (m, 1H), 3.08 (m, 1H), 2.84-2.69 (m, 2H), 2.01 (m, 1H), 1.76-1.65 (m, 1H), 1.13 (d, J=6.4 Hz, 3H).

### Intermediate G18: 5-Bromo-4-(difluoromethyl)-1-methylpyridin-2(1H)-one (G18)

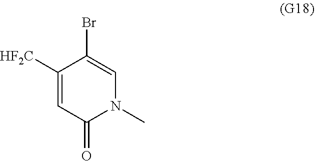

Step 1: To a solution of methyl 5-bromo-2-oxo-1,2-dihydropyridine-4-carboxylate (20.0 g, 86.2 mmol) in DMF (300 mL) was added Cs₂CO₃ (84.0 g, 258.6 mmol, 3 eq.), followed by CH₃I (31.8 g, 224 mmol, 2.6 eq.) at 20° C. The mixture was stirred at 20° C. for 15 hours. The mixture was poured into H₂O (1000 mL) and then extracted with ethyl acetate (300 mL×4). The combined organic phases were dried over Na₂SO₄, filtered and concentrated. The residue was re-crystallized from petroleum ether (100 mL) to afford methyl 5-bromo-1-methyl-2-oxo-1,2-dihydropyridine-4-carboxylate (15 g, 61.4% yield) as a light yellow solid. ¹H NMR (400 MHz, DMSO-d6) δ 8.20 (s, 1H), 6.72 (s, 1H), 3.85 (s, 3H), 3.45 (s, 3H).

Step 2: To a solution of methyl 5-bromo-1-methyl-2-oxo-1,2-dihydropyridine-4-carboxylate (From step 1, 3.5 g, 14.22 mmol) in EtOH (52 mL) was added LiCl (1.81 g, 42.70 mmol, 3.0 eq.) and NaBH₄ (2.69 g, 71.11 mmol, 5.0 eq.) at 0° C. The resulting mixture was stirred at 20° C. for 16 hours. Saturated aqueous NH₄Cl solution (200 mL) was added to the mixture and the mixture was extracted with DCM (50 mL×10). The combined organic layers were dried over anhydrous Na₂SO₄, filtered and concentrated to afford 5-Bromo-4-(hydroxymethyl)-1-methylpyridin-2(1)-one (1.25 g, 40.3% yield) as a white solid. ¹H NMR (400 MHz, CD₃OD) δ 7.88 (s, 1H), 6.73 (s, 1H), 4.48 (s, 2H), 3.54 (s, 3H).

Step 3: To a solution of 5-bromo-4-(hydroxymethyl)-1-methylpyridin-2(1)-one (From step 2, 1.25 g, 5.73 mmol) in DCM (25 mL) was added Dess-Martin periodinane (6.08 g, 14.33 mmol, 2.5 eq.) at 0° C. The reaction mixture was stirred at 0° C. for 10 minutes. The mixture was purified by column chromatography on silica gel (petroleum ether:ethyl acetate=10:1˜0:1) to afford 5-Bromo-1-methyl-2-oxo-1,2-dihydropyridine-4-carbaldehyde (500 mg, 40.4% yield) as a yellow solid. ¹H NMR (400 MHz, CDCl₃) δ 10.13 (s, 1H), 7.61 (s, 1H), 7.04 (s, 1H), 3.59 (s, 3H).

Step 4: To a solution of 5-bromo-1-methyl-2-oxo-1,2-dihydropyridine-4-carbaldehyde (From step 3, 500 mg, 2.31 mmol) in DCM (10 mL) was added DAST (937.1 mg, 5.78 mmol, 2.5 eq.) at −70° C. The mixture was stirred at −70° C. for 10 minutes and then warmed to 25° C. The mixture was further stirred at 25° C. for 30 minutes. The mixture was poured into saturated aqueous NaHCO₃ solution (160 mL) and was then extracted with DCM (50 mL×3). The combined organic layers were dried over anhydrous Na₂SO₄, filtered and concentrated to afford 5-bromo-4-(difluoromethyl)-1-methylpyridin-2(1H)-one (G18) (300 mg, 54.4% yield) as a yellow solid. ¹H NMR (400 MHz, CDCl₃) δ 7.55 (s, 1H), 6.87 (s, 1H), 6.74-6.41 (t, J=56 Hz, 1H), 3.56 (s, 3H).

### Intermediate G19: 5-Bromo-3-fluoro-1,4-dimethylpyridin-2(1H)-one (G19)

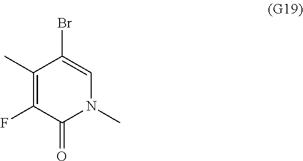

Step 1: To a solution of 4-methylpyridin-2(1 h)-one (2.0 g, 18.33 mmol) in CHCl₃/H₂O (V/V=1/1, 40 mL) was added Selectfluor (6.5 g, 18.33 mmol, 1.0 eq.) at 25° C. The reaction mixture was stirred at 35° C. for 16 hours. The reaction mixture was diluted with brine (100 mL) and was extracted with DCM (50 mL×5). The combined organic phases were dried over anhydrous Na₂SO₄, filtered and concentrated. The residue was purified by prep-HPLC (0.1% NH₃.H₂O) to afford 3-fluoro-4-methylpyridin-2(1H)-one (270 mg, 11% yield) as a yellow solid. ¹H NMR (400 MHz, CDCl3) δ 13.45 (br s, 1H), 7.14 (d, J=6.4 Hz, 1H), 6.14-6.11 (m, 1H), 2.24 (d, J=2.4 Hz, 3H). LCMS (ESI) m/z 128.0 [M+H]⁺.

Step 2: To a solution of 3-fluoro-4-methylpyridin-2(1H)-one (From step 1, 270 mg, 2.12 mmol) in CH₃CN (5 mL) was added K₂CO₃ (587 mg, 4.25 mmol, 2.0 eq.) and MeI (452 mg, 0.2 mL, 3.19 mmol, 1.5 eq.) at 25° C. The mixture was stirred at 70° C. for 16 hours. The reaction mixture was diluted with H₂O (10 mL) and extracted with EtOAc (5 mL×3). The combined organic phases were dried over anhydrous Na₂SO₄, filtered and concentrated to afford 3-fluoro-1,4-dimethylpyridin-2(1H)-one (270 mg, 90% yield) as a light yellow solid. ¹H NMR (400 MHz, CDCl₃) δ 6.99-6.97 (m, 1H), 5.98 (t, J=6.4 Hz, 1H), 3.56 (s, 3H), 2.18 (d, J=2.4 Hz, 3H). LCMS (ESI) m/z 142.0 [M+H]⁺.

Step 3: To a solution of 3-fluoro-1,4-dimethylpyridin-2(1H)-one (From step 2, 240 mg, 1.70 mmol) in CHCl₃ (6 mL) was added Br₂ (543 mg, 0.17 mL, 3.40 mmol) in CHCl₃ (6 mL) dropwise at 25° C. The mixture was stirred at 25° C. for 16 hours. Saturated Na₂SO₃ solution (30 mL) was added to the mixture and the mixture was extracted with DCM (20 mL×3). The combined organic phases were dried over anhydrous Na₂SO₄, filtered and concentrated. The residue was purified by silica gel column chromatography (petroleum ether/ethyl acetate=10/1 to 1/1) to afford the 5-bromo-3-fluoro-1,4-dimethylpyridin-2(1)-one (G19) (200 mg, 44% yield) as a white solid. ¹H NMR (400 MHz, CDCl₃) δ 7.32 (d, J=1.6 Hz, 1H), 3.57 (s, 3H), 2.25 (d, J=3.2 Hz, 3H). LCMS (ESI) m/z 220.0 [M+H]⁺.

### Intermediate G20: 6-Bromo-2-methyl-5-(trifluoromethyl)pyridazin-3(2H)-one (G20)

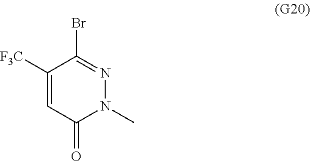

Step 1: A mixture of 3-(trifluoromethyl)furan-2,5-dione (5.0 g, 0.03 mol) and methylhydrazine sulfate (4.77 g, 0.33 mol) in acetic acid (50 mL) was heated to reflux for 2 hours. The mixture was cooled to room temperature and solvent was removed under vacuum to give the crude product. The crude product was then purified by flash column chromatography on silica gel (petroleum ether/ethyl acetate=1/1) to give 1-Methyl-4-(trifluoromethyl)-1,2-dihydropyridazine-3,6-dione (1.95 g, 34% yield) as a white solid. ¹H NMR (400 MHz, DMSO-d6) δ 12.21 (s, 1H), 7.38 (s, 1H), 3.52 (s, 3H). LCMS (ESI) m/z 194.9 [M+H]⁺.

Step 2: To a solution of 1-methyl-4-(trifluoromethyl)-1,2-dihydropyridazine-3,6-dione (From step 1, 1.0 g, 5.15 mmol) in 1,2-dichloroethane (30 mL) was added POBr₃ (2.95 g, 10.30 mmol) at room temperature. The mixture was stirred at 100° C. for 12 hours. The resulting mixture was cooled to room temperature and solvent was removed under vacuum to give crude product. The crude product was purified by flash column chromatography on silica gel (petroleum ether/ethyl acetate=3/2) to give 6-bromo-2-methyl-5-(trifluoromethyl)pyridazin-3(2H)-one (G20) (550 mg, 42% yield) as an orange solid. ¹H NMR (400 MHz, DMSO-d₆) δ 7.67-7.48 (m, 1H), 3.67 (s, 3H). LCMS (ESI) m/z 256.8 [M+H]⁺.

### Exemplary Compounds

### Example 1: Synthesis of 6-(1-(5-bromo-7-((2-(methylamino)-1H-imidazol-1-yl)methyl)-1-oxo-3,4 dihydroisoquinolin-2(1H)-yl)ethyl)-4-ethoxynicotinonitrile (1)

6-(1-(5-bromo-7-((2-(methylamino)-1H-imidazol-1-yl)methyl)-1-oxo-3,4 dihydroisoquinolin-2(1H)-yl)ethyl)-4-ethoxynicotinonitrile (1) was obtained by NaH mediated SN2 reaction as described below.

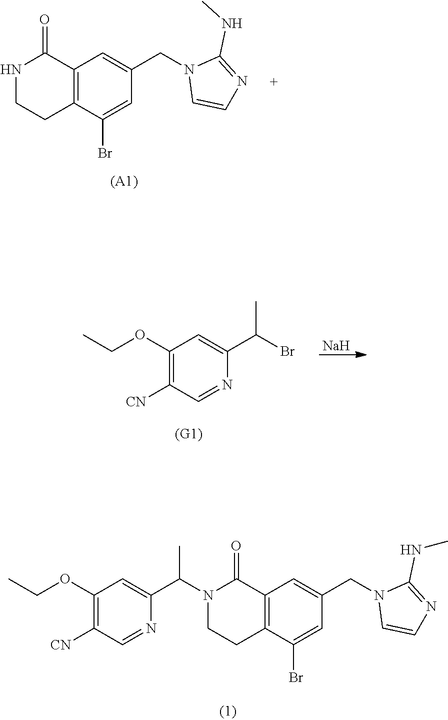

To a solution of intermediate (A1) (1.16 g, 3.46 mmol, 1.0 eq) in N, N-dimethyl formamide (10 mL) was added NaH (0.3 g, 7.6 mmol, 2.2 eq, 60% purity in oil) at 0° C. under nitrogen atmosphere. The mixture was stirred at 0° C. for 15 min. Then to the mixture was added intermediate (G1) (1.07 g, 4.19 mmol, 1.2 eq) in N, N-dimethyl formamide (3 mL) at 0° C. and the resulting mixture was stirred at rt for 15 min. LCMS showed the reaction was complete. The mixture was filtered and the filtrate was concentrated and purified by flash chromatography on silica gel (20% MeOH in CH₂Cl₂) to afford 6-(1-(5-bromo-7-((2-(methylamino)-1H-imidazol-1-yl)methyl)-1-oxo-3,4 dihydroisoquinolin-2(1H)-yl)ethyl)-4-ethoxynicotinonitrile (1) (442.5 mg, 24.2% yield) as a brown solid. ¹H NMR (400 MHz, Methanol-d₄) δ=8.61 (br s, 1H), 8.52 (br s, 1H), 7.79 (br s, 1H), 7.66 (br s, 1H), 7.18 (br s, 1H), 6.90 (br s, 2H), 5.91 (br d, J=6.8 Hz, 1H), 5.08 (br s, 2H), 4.31 (br d, J=6.7 Hz, 2H), 3.70-3.51 (m, 3H), 3.05 (br dd, J=6.7, 13.8 Hz, 2H), 2.96 (s, 3H), 1.66 (br d, J=6.7 Hz, 3H), 1.47 (br t, J=6.2 Hz, 3H). LC-MS: [M+H]⁺=508.9.

### Example 2: Synthesis of 5-((5-bromo-7-((2-(methylamino)-1H-imidazol-1-yl)methyl)-1-oxo-3,4-dihydroisoquinolin-2(1H)-yl)methyl)-3-ethoxypicolinonitrile (2)

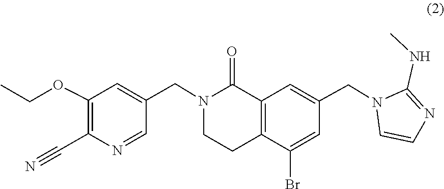 [SMILES: CCOc1cc(CN2CCc3c(Br)cc(Cn4ccnc4NC)cc3C2=O)cnc1C#N]

5-((5-bromo-7-((2-(methylamino)-1H-imidazol-1-yl)methyl)-1-oxo-3,4-dihydroisoquinolin-2(1H)-yl)methyl)-3-ethoxypicolinonitrile (2) was obtained using a synthesis similar to Example 1, except intermediate (G1) was replaced with intermediate (G4). ¹H NMR (400 MHz, Methanol-d₄) δ ppm 8.23 (d, J=1.5 Hz, 1H), 7.83 (s, 1H), 7.61 (s, 2H), 6.78 (d, J=6.0 Hz, 2H), 5.03 (s, 2H), 4.84 (s, 2H), 4.24 (q, J=7.0 Hz, 2H), 3.67 (t, J=6.7 Hz, 2H), 3.14-3.09 (m, 2H), 2.91 (s, 3H), 1.46 (t, J=7.0 Hz, 3H). LC-MS: [M+H]⁺=497.0.

### Example 3: Synthesis of (S)-6-(1-(5-chloro-7-((2-(methylamino)-1H-imidazol-1-yl)methyl)-1-oxo-3,4-dihydroisoquinolin-2(1H)-yl)ethyl)-4-ethoxynicotinonitrile (3)

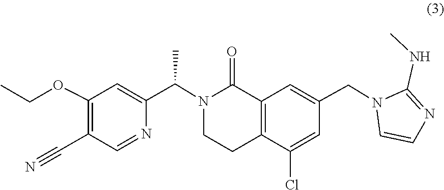 [SMILES: CCOc1cc([C@H](C)N2CCc3c(Cl)cc(Cn4ccnc4NC)cc3C2=O)ncc1C#N]

The racemic mixture of 6-(1-(5-chloro-7-((2-(methylamino)-1H-imidazol-1-yl)methyl)-1-oxo-3,4-dihydroisoquinolin-2(1H)-yl)ethyl)-4-ethoxynicotinonitrile was obtained using a synthesis similar to Example 1, except intermediate (A1) was replaced with intermediate (A2). The racemic mixture was separated by SFC (Column: (R,R)-WHELKO1, 5 um, 4.6 mm*250 mm, Condition: ETOH+0.5% NH4OH) to give example (3) (the 2nd peak on the prep SFC) together with its enantiomer. (S)-6-(1-(5-chloro-7-((2-(methylamino)-1H-imidazol-1-yl)methyl)-1-oxo-3,4-dihydroisoquinolin-2(1H)-yl)ethyl)-4-ethoxynicotinonitrile (3) ¹H NMR (400 MHz, Methanol-d₄) δ 8.62 (s, 1H), 7.75 (s, 1H), 7.34 (d, J=1.5 Hz, 1H), 7.17 (s, 1H), 6.65 (d, J=1.7 Hz, 1H), 6.61 (d, J=1.7 Hz, 1H), 5.92 (q, J=7.1 Hz, 1H), 4.97 (s, 2H), 4.30 (q, J=7.0 Hz, 2H), 3.64 (ddd, J=13.8, 8.9, 5.1 Hz, 1H), 3.54 (ddd, J=12.7, 6.9, 5.5 Hz, 1H), 3.15-2.91 (m, 2H), 2.84 (s, 3H), 1.65 (d, J=7.1 Hz, 3H), 1.46 (t, J=7.0 Hz, 3H). LCMS: [M+H]⁺=464.8.

### Example 4: Synthesis of (S)-5-(1-(5-chloro-7-((2-(methylamino)-1H-imidazol-1-yl)methyl)-1-oxo-3,4-dihydroisoquinolin-2(1H)-yl)ethyl)-3-ethoxypicolinonitrile (4)

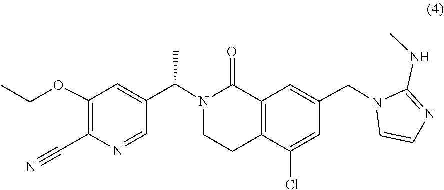 [SMILES: CCOc1cc([C@H](C)N2CCc3c(Cl)cc(Cn4ccnc4NC)cc3C2=O)cnc1C#N]

(S)-5-(1-(5-chloro-7-((2-(methylamino)-1H-imidazol-1-yl)methyl)-1-oxo-3,4-dihydroisoquinolin-2(1H)-yl)ethyl)-3-ethoxypicolinonitrile (4) was obtained using a synthesis similar to Example 3, except intermediate (G1) was replaced with intermediate (G3) and the racemic mixture separated by similar SFC condition. ¹H NMR (400 MHz, Methanol-d₄) δ ppm 8.27 (d, J=1.3 Hz, 1H), 7.78 (s, 1H), 7.58 (s, 1H), 7.38 (d, J=1.5 Hz, 1H), 6.69 (dd, J=14.1, 1.8 Hz, 2H), 6.03 (q, J=7.1 Hz, 1H), 5.00 (s, 2H), 4.26 (qd, J=7.0, 1.7 Hz, 2H), 3.66-3.55 (m, 1H), 3.36-3.32 (m, 1H), 3.15-3.01 (m, 1H), 3.01-2.89 (m, 1H), 2.87 (s, 3H), 1.68 (d, J=7.2 Hz, 3H), 1.46 (t, J=7.0 Hz, 3H). LC-MS: [M+H]⁺=465.0.

### Example 5: Synthesis of (S)-5-chloro-2-(1-(4-ethoxy-5-fluoropyridin-2-yl)ethyl)-7-((2-(methylamino)-1H-imidazol-1-yl)methyl)-3,4-dihydroisoquinolin-1(2H)-one (5)

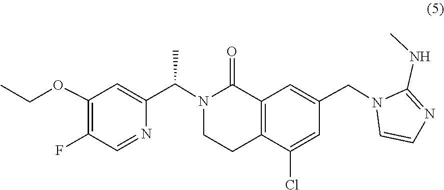 [SMILES: CCOc1cc([C@H](C)N2CCc3c(Cl)cc(Cn4ccnc4NC)cc3C2=O)ncc1F]

(S)-5-chloro-2-(1-(4-ethoxy-5-fluoropyridin-2-yl)ethyl)-7-((2-(methylamino)-1H-imidazol-1-yl)methyl)-3,4-dihydroisoquinolin-1(2H)-one (5) was obtained using a synthesis similar to Example 3, except intermediate (G1) was replaced with intermediate (G5) and the racemic mixture separated by similar SFC condition. ¹H NMR (400 MHz, Methanol-d₄) δ ppm 8.23 (d, J=3.3 Hz, 1H), 7.77 (d, J=1.4 Hz, 1H), 7.32 (d, J=1.6 Hz, 1H), 7.13 (d, J=6.9 Hz, 1H), 6.65 (d, J=1.7 Hz, 1H), 6.60 (d, J=1.7 Hz, 1H), 5.93 (q, J=7.1 Hz, 1H), 4.97 (s, 2H), 4.19 (qd, J=7.0, 2.0 Hz, 2H), 3.58 (ddd, J=13.9, 9.1, 5.0 Hz, 1H), 3.41 (ddd, J=12.7, 7.1, 5.4 Hz, 1H), 3.09-2.99 (m, 1H), 2.97-2.87 (m, 1H), 2.84 (s, 3H), 1.62 (d, J=7.1 Hz, 3H), 1.43 (t, J=7.0 Hz, 3H). LCMS: [M+H]+=458.1.

### Example 6: Synthesis of 5-((5-chloro-7-((2-(methylamino)-1H-imidazol-1-yl)methyl)-1-oxo-3,4-dihydroisoquinolin-2(1H)-yl)methyl)-3-ethoxypicolinonitrile (6)

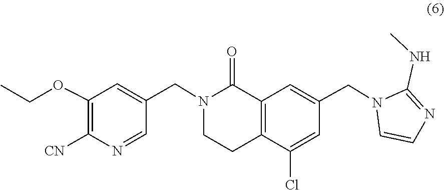

5-((5-chloro-7-((2-(methylamino)-1H-imidazol-1-yl)methyl)-1-oxo-3,4-dihydroisoquinolin-2(1H)-yl)methyl)-3-ethoxypicolinonitrile (6) was obtained using a synthesis similar to Example 1, except intermediate (A1) was replaced with intermediate (A2) and intermediate (G1) was replaced with intermediate (G4). ¹H NMR (400 MHz, Methanol-d₄) δ ppm 8.49 (s, 1H), 8.23 (d, J=1.3 Hz, 1H), 7.81-7.70 (m, 1H), 7.67-7.58 (m, 1H), 7.53 (d, J=1.3 Hz, 1H), 6.94 (q, J=2.3 Hz, 2H), 5.10 (s, 2H), 4.85 (s, 2H), 4.25 (q, J=7.0 Hz, 2H), 3.69 (t, J=6.7 Hz, 2H), 3.14 (t, J=6.7 Hz, 2H), 2.97 (s, 3H), 1.47 (t, J=7.0 Hz, 3H). LC-MS: [M+H]⁺=451.2.

### Example 7: Synthesis of (S)-2-(1-(5-fluoro-4-methoxypyridin-2-yl)ethyl)-7-((2-(methylamino)-1H-imidazol-1-yl)methyl)-5-(morpholinomethyl)-3,4-dihydroisoquinolin-1(2H)-one (7)

### Step 1: Synthesis of Potassium trifluoro(morpholinomethyl)borate (7-1)

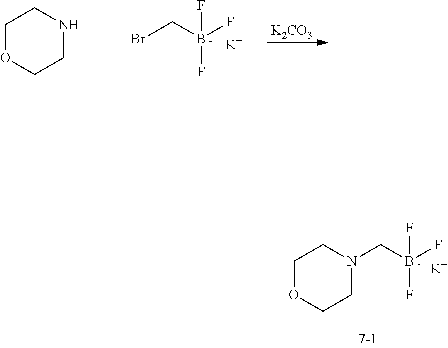

To a 100 mL of flask was added morpholine (868 mg, 9.96 mmol), potassium bromomethyltrifluoroborate (2000 mg, 9.96 mmol) and THF (30 mL). The mixture was heated and stirred at 80° C. for 3 h. The reaction mixture was concentrated under reduced pressure. The residue was dissolved in a solution of acetone (30 mL) and K₂CO₃ (1376 mg, 9.96 mmol) and stirred for 30 mins. The insoluble salts were filtered off, and the filtrate was concentrated under reduced pressure to give potassium trifluoro(morpholinomethyl)borate (7-1) (350 mg, 17%) as light yellow oil. ¹H NMR (400 MHz, Methanol-d4) δ 3.83 (s, 4H), 3.16 (s, 4H), 2.15-2.07 (m, 2H). LC-MS: [M+H]⁺=168.1.

### Step 2: Synthesis of (5-bromo-2-(1-(5-fluoro-4-methoxypyridin-2-yl)ethyl)-7-((2-(methylamino)-1H-imidazol-1-yl)methyl)-3,4-dihydroisoquinolin-1(2H)-one (7-2)

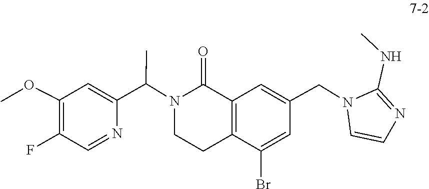 [SMILES: CNc1nccn1Cc1cc(Br)c2c(c1)C(=O)N(C(C)c1cc(OC)c(F)cn1)CC2]

5-bromo-2-(1-(5-fluoro-4-methoxypyridin-2-yl)ethyl)-7-((2-(methylamino)-1H-imidazol-1-yl)methyl)-3,4-dihydroisoquinolin-1(2H)-one (7-2) was obtained using the procedure described in Example 1 using intermediate (A1) but replacing intermediate (G1) with intermediate (G9). ¹H NMR (400 MHz, Methanol-d₄) δ ppm δ=8.23 (d, J=3.1 Hz, 1H), 7.82 (s, 1H), 7.51 (s, 1H), 7.16 (d, J=6.9 Hz, 1H), 6.65 (dd, J=1.7, 13.9 Hz, 2H), 5.94 (q, J=7.1 Hz, 1H), 4.98 (s, 2H), 3.98-3.91 (m, 3H), 3.63-3.38 (m, 2H), 3.10-2.79 (m, 5H), 1.62 (d, J=7.0 Hz, 3H). LCMS: [M+H]⁺=490.2.

### Step 3: Synthesis of (S)-2-(1-(5-fluoro-4-methoxypyridin-2-yl)ethyl)-7-((2-(methylamino)-1H-imidazol-1-yl)methyl)-5-(morpholinomethyl)-3,4-dihydroisoquinolin-1(2H)-one (7)

(S)-2-(1-(5-fluoro-4-methoxypyridin-2-yl)ethyl)-7-((2-(methylamino)-1H-imidazol-1-yl)methyl)-5-(morpholinomethyl)-3,4-dihydroisoquinolin-1(2H)-one (7) was obtained using the Molander coupling reaction as described below.

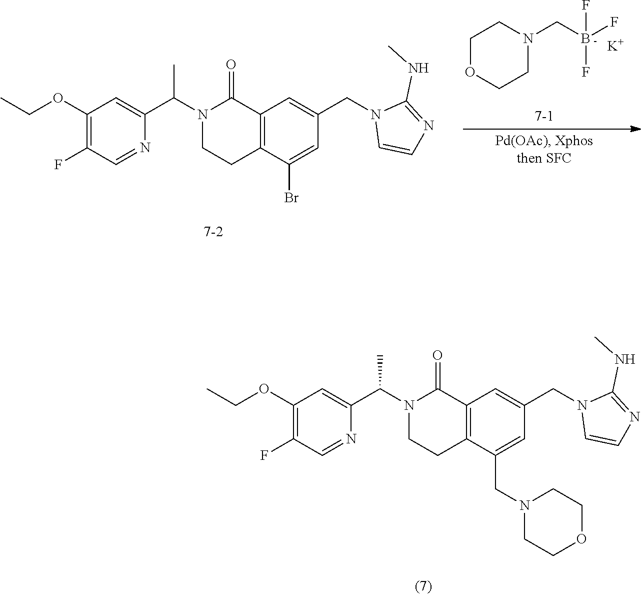

To a reaction tube was added intermediate (7-2) (30 mg, 0.061 mmol), intermediate (7-1) (22.89 mg, 0.111 mmol), diacetoxypalladium (2.069 mg, 9.21 μmol), Xphos (8.79 mg, 0.018 mmol) and Cs₂CO₃ (60.0 mg, 0.184 mmol). Under N₂, THF (1.5 mL) and water (0.150 mL) were added and the mixture stirred at 85° C. for 16 hrs. Water was then added and the pH was adjusted to 8-9 with Na₂CO₃. The mixture was separated and the aqueous phase was extracted with ethyl acetate. The combined organic layers were dried over anhydrous Na₂SO₄, filtered and concentrated. The residue was purified by prep-HPLC (Column: Luna C18 150*25 5 u, gradient: 15-42% of B, A=water (0.225% FA)-ACN, B=acetonitrile, flow rate: 100 mL/min) to give a racemic mixture (12.3 mg, 36.1%) which was separated by chiral SFC (Column: (R,R)-WHELKO1, 5 um, 4.6 mm*250 mm, Condition: ETOH+0.5% NH4OH) to give (S)-2-(1-(5-fluoro-4-methoxypyridin-2-yl)ethyl)-7-((2-(methylamino)-1H-imidazol-1-yl)methyl)-5-(morpholinomethyl)-3,4-dihydroisoquinolin-1(2H)-one (7) (the 2nd peak on the prep SFC) together with its enantiomer. ¹H NMR (400 MHz, Methanol-d₄) δ ppm 8.24 (d, J=3.2 Hz, 1H), 7.79 (d, J=1.6 Hz, 1H), 7.23 (d, J=1.6 Hz, 1H), 7.17 (d, J=6.9 Hz, 1H), 6.61 (dd, J=10.8, 1.7 Hz, 2H), 5.98 (q, J=7.0 Hz, 1H), 4.96 (s, 2H), 3.94 (s, 3H), 3.66-3.58 (m, 4H), 3.53 (d, J=9.0 Hz, 1H), 3.48 (s, 2H), 3.42-3.36 (m, 1H), 3.03 (d, J=6.9 Hz, 1H), 3.00-2.89 (m, 1H), 2.84 (s, 3H), 2.38 (s, 4H), 1.63 (d, J=7.1 Hz, 3H). LC-MS: [M+H]⁺=509.2.

### Example 19: Synthesis of (S)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one (19)

### Step 1: Synthesis of 5-bromo-2-(1-(4-ethoxy-5-fluoropyridin-2-yl)ethyl)-7-((2-(methylamino)-1H-imidazol-1-yl)methyl)-3,4-dihydroisoquinolin-1(2H)-one (19-1)

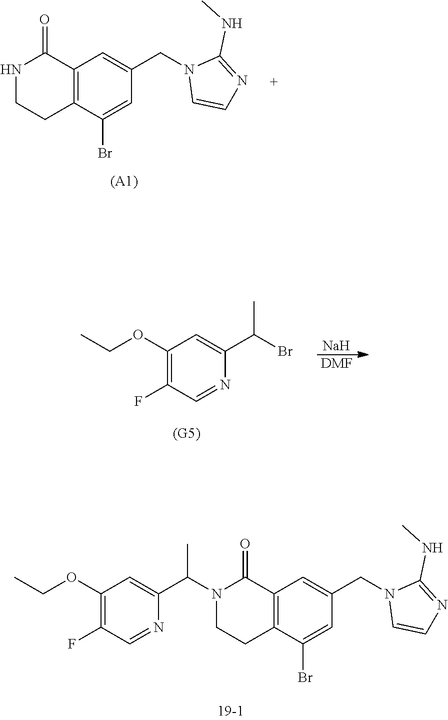

To a solution of intermediate (A1) (160 g, 477.3 mmol, 1.0 eq.) in dimethyl formamide (2400 mL) was added NaH (30.55 g, 763.7 mmol, 1.6 eq., 60% content in oil) at 0° C. under N₂. The reaction was stirred at 0° C. for 30 mins. The solution of intermediate G5 (153.9 g, 620.53 mmol, 1.3 eq.) in dimethyl formamide (600 mL) was added to the above mixture at 0° C. The resulting mixture was stirred at 5° C. for 30 mins. The reaction was quenched with methanol (500 mL) and water (7 L). The mixture was extracted with ethyl acetate (2000 mL×3). The combined organic layers were washed with brine (2 L), dried over Na₂SO₄, filtered and concentrated under reduced pressure. The residue was purified by column chromatography eluting with ethyl acetate/methanol (gradient: 2-10% of methanol) to afford 5-bromo-2-(1-(4-ethoxy-5-fluoropyridin-2-yl)ethyl)-7-((2-(methylamino)-1H-imidazol-1-yl)methyl)-3,4-dihydroisoquinolin-1(2H)-one (19-1) (141 g, 58.8% yield) as yellow solid. ¹H NMR (MeOD 400 MHz): δ 8.24 (d, J=3.3 Hz, 1H), 7.82 (s, 1H), 7.50 (d, J=1.5 Hz, 1H), 7.13 (d, J=6.8 Hz, 1H), 6.63 (dd, J=1.7, 18.6 Hz, 2H), 5.93 (q, J=7.1 Hz, 1H), 4.97 (s, 2H), 4.20 (dq, J=1.8, 7.0 Hz, 2H), 3.63-3.52 (m, 1H), 3.41 (ddd, J=5.4, 7.1, 12.7 Hz, 2H), 3.08-2.99 (m, 1H), 2.97-2.88 (m, 1H), 2.85 (s, 3H), 1.62 (d, J=7.1 Hz, 3H), 1.43 (t, J=7.0 Hz, 3H). LCMS: MS (ESI) m/z 502.1 [M+H]⁺.

### Step 2: Synthesis of (S)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one (19)

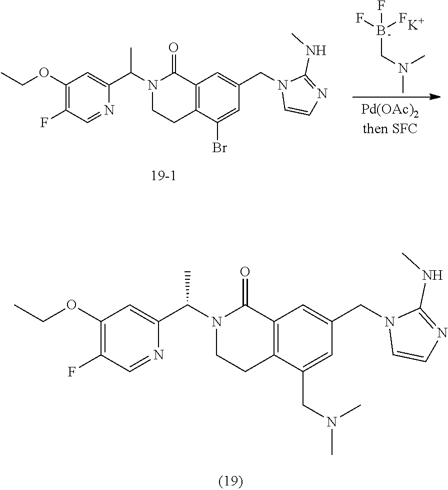

To a solution of compound 19-1 (120 g, 0.239 mol, 1.0 eq.) and potassium ((dimethylamino)methyl)trifluoroborate (352.45 g, 1.433 mol, 6.0 eq.) in tetrahydrofuran/water (2400 mL/240 mL) were added Xphos (34.2 g, 0.0716 mol, 0.3 eq.), Pd (AcO)₂ (8.04 g, 0.0358 mol, 0.15 eq.) and Cs₂CO₃ (233.5 g, 0.716 mmol, 3 eq.). The mixture was refluxed for 16 hrs under N₂ atmosphere. The reaction was added water (2.4 L) and the pH was adjusted to 8-9 with Na₂CO₃. The solution mixture separated and the aqueous phase was extracted with ethyl acetate (2 L*1). The combined organic layers were washed with brine (1 L*1), dried over anhydrous Na₂SO₄, filtered and concentrated. The residue was purified by column ethyl acetate/methanol (gradient: 0-20% Methanol, 1% Et₃N) to afford the crude. The crude was purified by prep-HPLC (Column: Phenomenex Synergi Max-RP250*50*10 um; Gradient: 1-25% of B, A=0.225% FA in water, B=acetonitrile, flow rate: 80 mL/min). The elution was adjusted to pH=9 with Na₂CO₃ and extracted with ethyl acetate (8 L×3). The combined organic layers were washed with brine (10 L), dried over anhydrous Na₂SO₄, filtered and concentrated to afford racemic compound (49 g, 42.7% yield) as yellow solid. The racemic compound was separated by SFC (Column: (R,R)-WHELKO1, 5 um, 4.6 mm*250 mm, Condition: ETOH+0.5% NH₄OH) to afford (S)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one (19) (second peak on SFC, 17.2 g) as yellow solid. ¹H NMR (MeOD 400 MHz): δ 8.24 (d, J=3.2 Hz, 1H), 7.81 (s, 1H), 7.22 (s, 1H), 7.13 (d, J=6.8 Hz, 1H), 6.60 (dd, J=1.3, 14.6 Hz, 2H), 5.97 (q, J=6.9 Hz, 1H), 4.96 (s, 2H), 4.24-4.14 (m, 2H), 3.58-3.49 (s, 1H), 3.42 (s, 2H), 3.40-3.33 (m, 1H), 3.06-2.97 (m, 1H), 2.95-2.87 (m, 1H), 2.84 (s, 3H), 2.20 (s, 6H), 1.62 (d, J=7.1 Hz, 3H), 1.42 (t, J=7.0 Hz, 3H). LCMS: MS (ESI) m/z 481.1 [M+H]⁺.

Following a similar procedure to that of Example 7 (unless otherwise specified). The following compounds were prepared from the corresponding precursors. The various borate compounds listed below were obtained using the procedure described in step 1 of Example 7 with the appropriate amine starting material.

| Ex. | Name/Structure | ¹HNMR / LC-MS | Precursors |
|---|---|---|---|
| 8 | 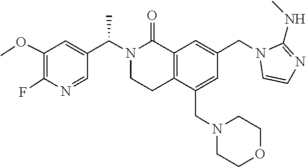 [SMILES: CNc1nccn1Cc1cc(CN2CCOCC2)c2c(c1)C(=O)N([C@@H](C)c1cnc(F)c(OC)c1)CC2] (S)-2-(1-(6-fluoro-5-methoxypyridin- 3-yl)ethyl)-7-((2-(methylamino)-1H- imidazol-1-yl)methyl)-5- (morpholinomethyl)-3,4- dihydroisoquinolin-1(2H)-one | ¹H NMR (400 MHz, Methanol-d₄) δ ppm 7.81 (s, 1H), 7.73 (s, 1H), 7.48 (d, J = 9.7 Hz, 1H), 7.24 (s, 1H), 6.68 -6.56 (m, 2H), 6.07 (q, J = 7.1 Hz, 1H), 4.97 (s, 2H), 3.89 (s, 3H), 3.65 - 3.58 (m, 4H), 3.58 -3.51 (m, 1H), 3.48 (d, J = 6.3 Hz, 3H), 3.26 - 3.17 (m, 1H), 3.13 - 3.02 (m, 1H), 2.92 (dd, J = 9.3, 5.2 Hz, 1H), 2.84 (s, 3H), 2.38 (s, 4H), 1.65 (d, J = 7.1 Hz, 3H). LC-MS: (ESI) m/z 509.2 [M + H]⁺ | Step 2: intermediate (A1) and intermediate (G7) Step 3: potassium trifluoro(morpholin omethyl)borate |
| 9 | 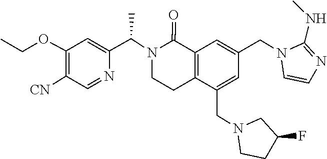 4-ethoxy-6-((S)-1-(5-(((S)-3- fluoropyrrolidin-1-yl)methyl)-7-((2- (methylamino)-1H-imidazol-1- yl)methyl)-1-oxo-3,4- dihydroisoquinolin-2(1H)- yl)ethyl)nicotinonitrile | ¹H NMR (400 MHz, Methanol-d₄) δ ppm 8.62 (s, 1H), 7.76 (s, 1H), 7.26 (s, 1H), 7.16 (s, 1H), 6.60 (dd, J = 13.6, 1.7 Hz, 2H), 5.95 (d, J = 7.1 Hz, 1H), 5.18 (s, 1H), 4.96 (s, 2H), 4.28 (q, J = 7.0 Hz, 2H), 3.65 (d, J = 8.6 Hz, 2H), 3.60 (s, 1H), 3.52 - 3.46 (m, 1H), 3.08 -2.96 (m, 2H), 2.84 (s, 3H), 2.81 - 2.73 (m, 2H), 2.63 (d, J = 25.3 Hz, 1H), 2.36 (d, J = 7.2 Hz, 1H), 2.20 - 2.15 (m, 1H), 2.02 (d, J = 5.7 Hz, 2H), 1.65 (d, J = 7.1 Hz, 3H), 1.45 (t, J = 7.0 Hz, 3H). LC-MS: (ESI) m/z 532.3 [M + H]⁺ | Step 2: intermediate (A1) and intermediate (G1) Step 3: potassium trifluoro(morpholin omethyl)borate was replaced with potassium (5)- trifluoro((3- fluoropyrrolidin-1- yl)methyl)borate |
| 10 | 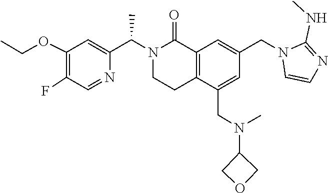 [SMILES: CCOc1cc([C@H](C)N2CCc3c(CN(C)C4COC4)cc(Cn4ccnc4NC)cc3C2=O)ncc1F] (S)-2-(1-(4-ethoxy-5-fluoropyridin-2- yl)ethyl)-5-((methyl(oxetan-3- yl)amino)methyl)-7-((2- (methylamino)-1H-imidazol-1- yl)methyl)-3,4-dihydroisoquinolin- 1(2H)-one | ¹H NMR (400 MHz, Methanol-d₄) δ ppm 8.24 (d, J = 3.3 Hz, 1H), 7.87 - 7.76 (m, 1H), 7.20 (d, J = 1.5 Hz, 1H), 7.13 (d, J = 6.9 Hz, 1H), 6.60 (dd, J = 13.0, 1.7 Hz, 2H), 5.96 (q, J = 7.1 Hz, 1H), 4.95 (s 2H) 4.52 (td J = 6.6 3.7 Hz 2H), 4.44 (q, J = 6.4 Hz, 2H), 4.19 (qd, J = 7.0, 2.4 Hz, 2H), 3.63 - 3.50 (m, 2H), 3.41 (dd, J = 7.0, 5.4 Hz, 1H), 3.37 (s, 2H), 3.18- 3.06 (m, 1H), 2.99 (dd, J = 9.3, 5.3 Hz, 1H), 2.84 (s, 3H), 1.99 (s, 3H), 1.62 (d, J = 7.1 Hz, 3H), 1.42 (t, J = 7.0 Hz, 3H). LC-MS: (ESI) m/z 523.0 [M + H]⁺ | Step 2: intermediate (A1) and intermediate (G5) Step 3: potassium trifluoro(morpholin omethyl)borate was replaced with potassium trifluoro((methyl(ox etan-3- yl)amino)methyl)b orate |
| 11 | 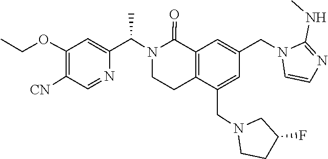 4-ethoxy-6-((S)-1-(5-(((R)-3- fluoropyrrolidin-1-yl)methyl)-7-((2- (methylamino)-1H-imidazol-1- yl)methyl)-1-oxo-3,4- dihydroisoquinolin-2(1H)- yl)ethyl)nicotinonitrile | ¹H NMR (400 MHz, Methanol-d₄) δ ppm 8.62 (s, 1H), 7.76 (s, 1H), 7.27 (s, 1H), 7.16 (s, 1H), 6.69- 6.52 (m, 2H), 5.95 (q, J = 7.0 Hz, 1H), 5.19 (s, 1H), 4.96 (s, 2H), 4.29 (q, J = 7.0 Hz, 2H), 3.65 (s, 2H), 3.58 (d, J = 8.8 Hz, 1H), 3.54 j - 3.47 (m, 1H), 3.14 - 3.04 (m, 1H), 2.99 (dd, J = 9.0, 5.2 Hz, 1H), 2.84 (s, 4H), 2.77 (d, J = 13.1 Hz, 1H), 2.64 (dd, J = 30.1, 5.0 Hz, 1H), 2.37 (d, J = 7.6 Hz, 1H), 2.22 - 2.15 (m, 1H), 2.02 (d, J = 5.9 Hz, 1H), 1.65 (d, J = 7.1 Hz, 3H), 1.45 (d, J = 7.0 Hz, 3H). LC-MS: (ESI) m/z 532.3 [M + H]⁺ | Step 2: intermediate (A1) and intermediate (G1) Step 3: potassium trifluoro(morpholin omethyl)borate was replaced with potassium (R)- trifluoro((3- fluoropyrrolidin-1- yl)methyl)borate |
| 12 | 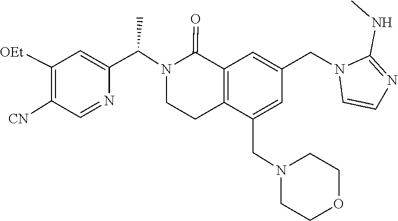 (S)-4-ethoxy-6-(1-(7-((2- (methylamino)-1H-imidazol-1- yl)methyl)-5-(morpholinomethyl)-1- oxo-3,4-dihydroisoquinolin-2(1H)- yl)ethyl)nicotinonitrile | ¹H NMR (400 MHz, Methanol-d₄) δ ppm 8.63 (s, 1H), 7.76 (d, J = 1.6 Hz, 1H), 7.24 (d, J = 1.7 Hz, 1H), 7.17 (s, 1H), 6.60 (dd, J = 11.2, 1.7 Hz, 2H), 5.95 (q, J = 7.1 Hz, 1H), 4.95 (s, 2H), 4.36 -4.24 (m, 2H), 3.61 (q, J = 6.4, 5.4 Hz, 5H), 3.50 (d, J = 9.3 Hz, 3H), 3.15 -2.90 (m, 2H), 2.83 (s, 3H), 2.39 (s, 4H), 1.65 (d, J = 7.1 Hz, 4H), 1.46 (t, J = 7.0 Hz, 3H). LC-MS: (ESI) m/z 530.3 [M + H]⁺ | Step 2: intermediate (A1) and intermediate (G1) Step 3: potassium trifluoro(morpholin omethyl)borate |
| 13 | 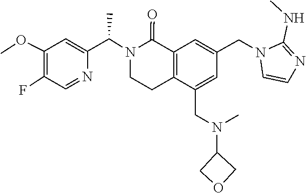 [SMILES: CNc1nccn1Cc1cc(CN(C)C2COC2)c2c(c1)C(=O)N([C@@H](C)c1cc(OC)c(F)cn1)CC2] (S)-2-(1-(5-fluoro-4-methoxypyridin- 2-ypethyl)-5-((methyl(oxetan-3- yl)amino)methyl)-7-((2- (methylamino)-1H-imidazol-1- yl)methyl)-3,4-dihydroisoquinolin- 1(2H)-one | ¹H NMR (400 MHz, Methanol-d₄) δ ppm 8.24 (d, J = 3.2 Hz, 1H), 7.81 (s, 1H), 7.23 (s, 1H), 7.16 (d, J = 6.9 Hz, 1H), 6.65 (dd, J = 9.2, 1.7 Hz, 2H), 5.98 (q, J = 7.2 Hz, 1H), 4.97 (s, 2H), 4.52 (td, J = 6.7, 2.1 Hz, 2H), 4.45 (q, J = 6.3 Hz, 2H), 3.94 (s, 3H), 3.63 - 3.51 (m, 2H), 3.43 (s, 1H), 3.39 (d, J = 11.7 Hz, 3H), 3.15 - 3.05 (m, 1H), 3.05 - 2.93 (m, 1H), 2.86 (s, 3H), 1.99 (s, 3H), 1.63 (d, J = 7.1 Hz, 3H). LC-MS: (ESI) m/z 509.3 [M + H]⁺ | Step 2: intermediate (A1) and intermediate (G9) Step 3: potassium trifluoro(morpholin omethyl)borate was replaced with potassium trifluoro((methyl(ox etan-3- yl)amino)methyl)b orate |
| 14 | 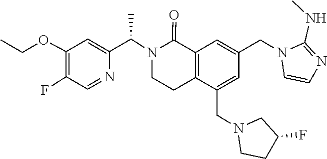 [SMILES: CCOc1cc([C@H](C)N2CCc3c(CN4CC[C@@H](F)C4)cc(Cn4ccnc4NC)cc3C2=O)ncc1F] 2-((S)-1-(4-ethoxy-5-fluoropyridin-2- yl)ethyl)-5-(((R)-3-fluoropyrrolidin-1- yl)methyl)-7-((2-(methylamino)-1H- imidazol-1-yl)methyl)-3,4- dihydroisoquinolin-1(2H)-one | ¹H NMR (400 MHz, Methanol-d₄) δ ppm 8.23 (d, J = 3.2 Hz, 1H), 7.79 (s, 1H), 7.25 (s, 1H), 7.12 (d, J = 6.9 Hz, 1H), 6.59 (dd, J = 12.6, 1.6 Hz, 2H), 5.96 (q, J = 7.1 Hz, 1H), 5.11 (d, J = 5.6 Hz, 1H), 4.95 (s, 2H), 4.18 (dd, J = 7.0, 3.2 Hz, 2H), 3.63 (d, J = 8.4 Hz, 2H), 3.53 (s, 1H), 3.37 (d, J = 7.0 Hz, 1H), 3.01 (d, J = 6.5 Hz, 1H), 2.93 (dd, J = 9.5, 5.4 Hz, 1H), 2.84 (s, 3H), 2.82 -2.70 (m, 2H), 2.63 (dd, J = 30.2, 5.0 Hz, 1H), 2.35 (d, J = 7.3 Hz, 1H), 2.17 (d, J = 7.3 Hz, 1H), 1.94 (ddd, J = 30.8, 13.6, 6.6 Hz, 1H), 1.61 (d, J = 7.1 Hz, 3H), 1.41 (t, J = 7.0 Hz, 3H). LC-MS: (ESI) m/z 525.5 [M + H]⁺ | Step 2: intermediate (A1) and intermediate (G5) Step 3: potassium trifluoro(morpholin omethyl)borate was replaced with potassium (R)- trifluoro((3- fluoropyrrolidin-1- yl)methyl)borate |
| 15 | 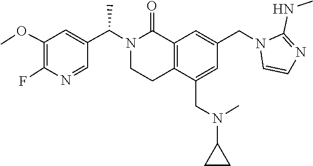 (S)-5- ((cyclopropyl(methyl)amino)methyl)- 2-(1-(6-fluoro-5-methoxypyridin-3- yl)ethyl)-7-((2-(methylamino)-1H- imidazol-1-yl)methyl)-3,4- dihydroisoquinolin-1(2H)-one | ¹H NMR (400 MHz, Methanol-d₄) δ ppm 7.82 (d, J = 1.9 Hz, 1H), 7.73 (d, J = 2.1 Hz, 1H), 7.48 (dd, J = 9.8, 2.1 Hz, 1H), 7.22 (d, J = 2.0 Hz, 1H), 6.60 (dd, J = 15.7, 1.7 Hz, 2H), 6.07 (q, J = 7.1 Hz, 1H), 4.96 (s, 2H), 3.89 (s, 3H), 3.65 (s, 2H), 3.55 - 3.44 (m, 1H), 3.20 (ddd, J = 12.4, 7.2, 5.0 Hz, 1H), 3.02 (ddd, J = 16.4, 7.4, 4.8 Hz, 1H), 2.83 (s, 4H), 2.20 (s, 3H), 1.73 - 1.61 (m, 4H), 0.41 (td, J = 6.6, 4.5 Hz, 2H), 0.30 - 0.23 (m, 2H). LC-MS: (ESI) m/z 493.3 [M + H]⁺ | Step 2: intermediate (A1) and intermediate (G7) Step 3: potassium trifluoro(morpholin omethyl)borate was replace with potassium ((cyclopropyl(meth yl)amino)methyl)tri fluoroborate |
| 16 | 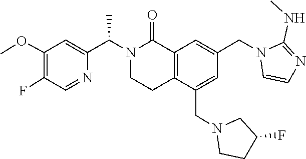 [SMILES: CNc1nccn1Cc1cc(CN2CC[C@@H](F)C2)c2c(c1)C(=O)N([C@@H](C)c1cc(OC)c(F)cn1)CC2] 2-((S)-1-(5-fluoro-4-methoxypyridin- 2-ypethyl)-5-(((R)-3-fluoropyrrolidin- 1-yl)methyl)-7-((2-(methylamino)-1H- imidazol-1-yl)methyl)-3,4- dihydroisoquinolin-1(2H)-one | ¹H NMR (400 MHz, Methanol-d₄) δ ppm 8.23 (d, J = 3.2 Hz, 1H), 7.79 (s, 1H), 7.25 (d, J = 1.4 Hz, 1H), 7.15 (d, J = 6.9 Hz, 1H), 6.59 (dd, J = 12.9, 1.7 Hz, 2H), 5.97 (q, J = 7.0 Hz, 1H), 5.22 - 4.98 (m, 1H), 4.95 (s, 2H), 3.93 (s, 3H), 3.63 (d, J = 1.6 Hz, 2H), 3.53 (dq, J = 9.2, 4.6 Hz, 1H), 3.43- 3.35 (m, 1H), 3.08 -2.98 (m, 1H), 2.93 (dd, J = 9.2, 5.2 Hz, 1H), 2.84 (s, 3H), 2.81 -2.73 (m, 2H), 2.63 (ddd, J = 30.0, 11.5, 5.0 Hz, 1H), 2.42 - 2.33 (m, 1H), 2.24- 2.04 (m, 1H), 1.93 (dd, J = 30.3, 7.0 Hz, 1H), 1.62 (d, J = 7.1 Hz, 3H). LC-MS: (ESI) m/z 511.3 [M + H]⁺ | Step 2: intermediate (A1) and intermediate (G9) Step 3: potassium trifluoro(morpholin omethyl)borate was replaced with potassium (R)- trifluoro((3- fluoropyrrolidin-1- yl)methyl)borate |
| 17 | 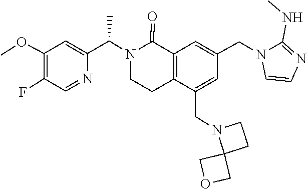 [SMILES: CNc1nccn1Cc1cc(CN2CCC23COC3)c2c(c1)C(=O)N([C@@H](C)c1cc(OC)c(F)cn1)CC2] (S)-5((6-oxa-1-azaspiro+3.3+heptan- 1-yl)methyl)-2-(1-(5-fluoro-4- methoxypyridin-2-yl)ethyl)-7-((2- (methylamino)-1H-imidazol-1- yl)methyl)-3,4-dihydroisoquinolin- 1(2H)-one | ¹H NMR (400 MHz, Methanol-d₄) δ ppm 8.24 (d, J = 3.0 Hz, 1H), 7.80 (s, 1H), 7.28 (s, 1H), 7.16 (d, J = 6.9 Hz, 1H), 6.60 (d, J = 12.5 Hz, 2H), 5.98 (d, J = 7.1 Hz, 1H), 4.96 (s, 2H), 4.92 (d, J = 7.4 Hz, 2H), 4.61 (d, J = 7.4 Hz, 2H), 3.94 (s, 3H), 3.85 (s, 2H), 3.54 (d, J = 8.9 Hz, 1H), 3.45 - 3.38 (m, 1H), 2.99 (d, J = 6.9 Hz, 4H), 2.83 (s, 3H), 2.33 (t, J = 6.7 Hz, 2H), 1.63 (d, J = 7.1 Hz, 4H). LC-MS: (ESI) m/z 521.3 [M + H]⁺ | Step 2: intermediate (A1) and intermediate (G9) Step 3: potassium trifluoro(morpholin omethyl)borate was replaced with potassium ((6-oxa- 1- azaspiro[3.3]hepta n-1- yl)methyl)trifluorob orate |
| 18 | 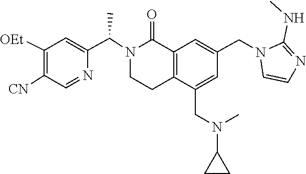 (S)-6-(1-(5- ((cyclopropyl(methyl)amino)methyl)- 7-((2-(methylamino)-1H-imidazol-1- yl)methyl)-1-oxo-3,4- dihydroisoquinolin-2(1H)-yl)ethyl)-4- ethoxynicotinonitrile | ¹H NMR (400 MHz, Methanol-d₄) δ ppm 8.62 (s, 1H), 7.77 (s, 1H), 7.22 (s, 1H), 7.16 (s, 1H), 6.59 (dd, J = 12.5, 1.5 Hz, 2H), 5.94 (d, J = 7.1 Hz, 1H), 4.95 (s, 2H), 4.29 (q, J = 7.0 Hz, 2H), 3.66 (s, 2H), 3.63 - 3.42 (m, 2H), 3.11- 2.87 (m, 2H), 2.83 (s, 3H), 2.20 (s, 3H), 1.66 (dd, J = 14.8, 5.3 Hz, 4H), 1.45 (t, J = 7.0 Hz, 3H), 0.46 - 0.36 (m, 2H), 0.27 (q, J = 3.3, 2.6 Hz, 2H). LC-MS:(ESI) m/z 514.3 [M + H]⁺ | Step 2: intermediate (A1) and intermediate (G1) Step 3: potassium trifluoro(morpholin omethyl)borate was replaced with potassium ((cyclopropyl(meth yl)amino)methyl)tri fluoroborate |
| 20 | 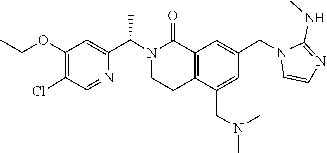 [SMILES: CCOc1cc([C@H](C)N2CCc3c(CN(C)C)cc(Cn4ccnc4NC)cc3C2=O)ncc1Cl] (S)-2-(1-(5-chloro-4-ethoxypyridin-2- yl)ethyl)-5-((dimethylamino)methyl)- 7-((2-(methylamino)-1H-imidazol-1- yl)methyl)-3,4-dihydroisoquinolin- 1(2H)-one | ¹H NMR (400 MHz, Methanol-d₄) δ ppm 8.34 (s, 1H), 7.80 (d, J = 1.9 Hz, 1H), 7.22 (d, J = 1.9 Hz, 1H), 7.07 (s, 1H), 6.60 (dd, J = 15.4, 1.8 Hz, 2H), 5.96 (q, J = 7.1 Hz, 1H), 4.96 (s, 2H), 4.20 (qd, J = 7.0, 2.4 Hz, 2H), 3.67 - 3.50 (m, 1H), 3.41 (d, J = 11.7 Hz, 3H), 3.07- 2.86 (m, 2H), 2.84 (s, 3H), 2.20 (s, 6H), 1.62 (d, J = 7.1 Hz, 3H), 1.44 (t, J = 7.0 Hz, 3H). LC-MS: (ESI) m/z 497.3 [M + H]⁺ | Step 2: intermediate (A1) and intermediate (G11) Step 3: potassium trifluoro(morpholin omethyl)borate was replaced with potassium ((dimethylamino)m ethyl)trifluoroborate |
| 21 | 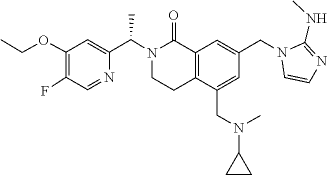 [SMILES: CCOc1cc([C@H](C)N2CCc3c(CN(C)C4CC4)cc(Cn4ccnc4NC)cc3C2=O)ncc1F] (S)-5- ((cyclopropyl(methyl)amino)methyl)- 2-(1-(4-ethoxy-5-fluoropyridin-2- yl)ethyl)-7-((2-(methylamino)-1H- imidazol-1-yl)methyl)-3,4- dihydroisoquinolin-1(2H)-one | ¹H NMR (400 MHz, Methanol-d₄) δ ppm 8.23 (d, J = 3.3 Hz, 1H), 7.79 (d, J = 1.6 Hz, 1H), 7.22 (d, J = 1.8 Hz, 1H), 7.12 (d, J = 6.9 Hz, 1H), 6.60 (dd, J = 13.3, 1.7 Hz, 2H), 5.96 (q, J = 7.1 Hz, 1H), 4.96 (s, 2H), 4.19 (qd, J = 7.0, 3.5 Hz, 2H), 3.66 (s, 2H), 3.58 - 3.46 (m, 1H), 2.99 (dd, J = 7.9, 3.3 Hz, 1H), 2.84 (s, 4H), 2.20 (s, 3H), 1.68 (s, 1H), 1.61 (d, J = 7.1 Hz, 3H), 1.42 (t, J = 7.0 Hz, 3H), 0.41 (dd, J = 6.6, 1.8 Hz, 2H), 0.28 - 0.20 (m, 2H). LC-MS: (ESI) m/z 507.3 [M + H]⁺ | Step 2: intermediate (A1) and intermediate (G5) Step 3: potassium trifluoro(morpholin omethyl)borate was replaced with potassium ((cyclopropyl(meth yl)amino)methyl)tri fluoroborate |
| 22 | 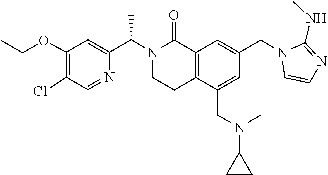 [SMILES: CCOc1cc([C@H](C)N2CCc3c(CN(C)C4CC4)cc(Cn4ccnc4NC)cc3C2=O)ncc1Cl] (S)-2-(1-(5-chloro-4-ethoxypyridin-2- yl)ethyl)-5- ((cyclopropyl(methyl)amino)methyl)- 7-((2-(methylamino)-1H-imidazol-1- yl)methyl)-3,4-dihydroisoquinolin- 1(2H)-one | ¹H NMR (400 MHz, Methanol-d₄) δ ppm 8.34 (s, 1H), 7.79 (d, J = 1.6 Hz, 1H), 7.22 (d, J = 1.7 Hz, 1H), 7.07 (s, 1H), 6.59 (dd, J = 14.1, 1.7 Hz, 2H), 5.96 (d, J = 7.1 Hz, 1H), 4.95 (s, 2H), 4.20 (dd, J = 7.0, 3.1 Hz, 2H), 3.66 (s, 2H), 3.54 - 3.44 (m, 1H), 3.37 (s, 1H), 3.05 -2.96 (m, 1H), 2.91 (dd, J = 9.3, 5.3 Hz, 1H), 2.83 (s, 3H), 2.20 (s, 3H), 1.68 (s, 1H), 1.62 (d, J = 7.1 Hz, 3H), 1.44 (t, J = 7.0 Hz, 3H), 0.41 (dd, J = 6.5, 1.8 Hz, 2H), 0.30 -0.21 (m, 2H). LC-MS: (ESI) m/z 523.3 [M + H]⁺ | Step 2: intermediate (A1) and intermediate (G11) Step 3: potassium trifluoro(morpholin omethyl)borate was replaced with potassium ((cyclopropyl(meth yl)amino)methyl)tri fluoroborate |
| 23 | 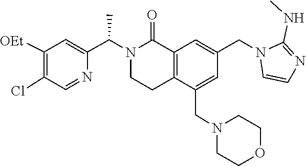 [SMILES: CCOc1cc([C@H](C)N2CCc3c(CN4CCOCC4)cc(Cn4ccnc4NC)cc3C2=O)ncc1Cl] (S)-2-(1-(5-chloro-4-ethoxypyridin-2- yl)ethyl)-7-((2-(methylamino)-1H- imidazol-1-yl)methyl)-5- (morpholinomethyl)-3,4- dihydroisoquinolin-1(2H)-one | ¹H NMR (400 MHz, Methanol-d₄) δ ppm 8.50 (s, 1H), 7.91 (s, 1H), 7.87 (d, J = 1.6 Hz, 1H), 7.29 (s, 1H), 7.00 (d, J = 2.6 Hz, 2H), 5.87 (q, J = 7.0 Hz, 1H), 5.19 (s, 2H), 4.51 (s, 2H), 4.36 (d, J = 6.9 Hz, 2H), 4.01 (s, 4H), 3.70 (ddd, J = 13.2, 8.7, 5.0 Hz, 1H), 3.59 (dt, J = 12.5, 6.0 Hz, 1H), 3.43 (s, 2H), 3.19 (s, 2H), 2.99 (s, 3H), 2.65 (s, 3H), 1.71 (d, J = 7.1 Hz, 3H), 1.49 (t, J = 7.0 Hz, 3H). LC-MS: (ESI) m/z 539.3 [M + H]⁺ | Step 2: intermediate (A1) and intermediate (G11) Step 3: potassium trifluoro(morpholin omethyl)borate was replaced with potassium trifluoro(morpholin omethyl)borate |
| 24 | 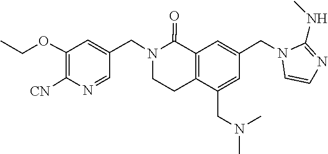 5-((5-((dimethylamino)methyl)-7-((2- (methylamino)-1H-imidazol-1- yl)methyl)-1-oxo-3,4- dihydroisoquinolin-2(1H)-yl)methyl)- 3-ethoxypicolinonitrile | ¹H NMR (400 MHz, Methanol-d₄) δ ppm 8.24 (d, J = 1.2 Hz, 1H), 7.96 (s, 1H), 7.65 (s, 2H), 7.00 (d, J = 2.2 Hz, 1H), 6.94 (d, J = 2.1 Hz, 1H), 5.16 (s, 2H), 4.87 (s, 2H), 4.44 (s, 2H), 4.25 (q, J = 7.0 Hz, 2H), 3.70 (t, J = 6.3 3.17 (t, J = 6.2 Hz, 2H) Hz, 2H), , 3H), 2.89 (s, 6H), 1.47 (t, J = 7.0 Hz, 3H). LC-MS: (ESI) m/z 473.9 [M + H]⁺ | Step 2: intermediate (A1) and intermediate (G4) Step 3: potassium trifluoro(morpholin omethyl)borate was replaced with potassium ((dimethylamino)m ethyl)trifluoroborat e |
| 25 | 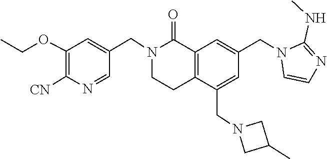 3-ethoxy-5-((7-((2-(methylamino)- 1H-imidazol-1-yl)methyl)-5-((3- methylazetidin-1-yl)methyl)-1-oxo- 3,4-dihydroisoquinolin-2(1H)- yl)methyl)picolinonitrile | ¹H NMR (400 MHz, Methanol-d₄) δ ppm 8.23 (d, J = 1.2 Hz, 1H), 7.86 (s, 1H), 7.64 (s, 1H), 7.45 (s, 1H), 6.96 (d, J = 2.1 Hz, 1H), 6.91 (d, J = 2.1 Hz, 1H), 5.12 (s, 2H), 4.86 (s, 2H), 4.25 (q, J = 7.0 Hz, 2H), 4.11 (s, 2H), 3.87 (d, J = 7.4 Hz, 2H), 3.67 (t, J = 6.2 Hz, 2H), 3.40 (t, J = 7.3 Hz, 2H), 3.09 (t, J = 6.2 Hz, 2H), 2.98 (s, 3H), 2.81 (s, 1H), 1.47 (t, J = 7.0 Hz, 3H), 1.23 (d, J = 6.6 Hz, 3H). LC-MS: (ESI) m/z 500.0 [M + H]⁺ | Step 2: intermediate (A1) and intermediate (G4) Step 3: potassium trifluoro(morpholin omethyl)borate was replaced with potassium trifluoro((3- methylazetidin-1- yl)methyl)borate |
| 26 | 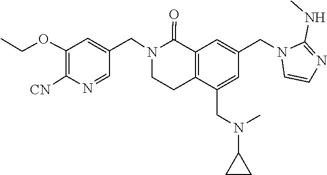 5-((5- ((cyclopropyl(methyl)amino)methyl)- 7-((2-(methylamino)-1H-imidazol-1- yl)methyl)-1-oxo-3,4- dihydroisoquinolin-2(1H)-yl)methyl)- 3-ethoxypicolinonitrile | ¹H NMR (400 MHz, Methanol-d₄) δ ppm 8.36 (s, 1H), 8.22 (d, J = 1.3 Hz, 1H), 7.78 (s, 1H), 7.62 (s, 1H), 7.39 (s, 1H), 6.94 (dd, J = 17.5, 2.3 Hz, 2H), 5.09 (s, 2H), 4.84 (s, 2H), 4.24 (q, J = 7.0 Hz, 2H), 3.73 (s, 2H), 3.62 (t, J = 6.6 Hz, 2H), 3.09 (t, J = 6.6 Hz, 2H), 2.98 (s, 3H), 2.65 (s, 3H), 2.25 (s, 3H), 1.80 - 1.70 (m, 1H), 1.45 (d, J = 7.0 Hz, 3H), 0.44 (d, J = 5.0 Hz, 2H), 0.29 (s, 2H). LC-MS: (ESI) m/z 499.9 [M + H]⁺ | Step 2: intermediate (A1) and intermediate (G4) Step 3: potassium trifluoro(morpholin omethyl)borate was replaced with potassium ((cyclopropyl(meth yl)amino)methyl)tri fluoroborate |
| 27 | 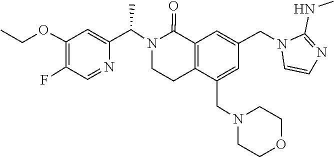 [SMILES: CCOc1cc([C@H](C)N2CCc3c(CN4CCOCC4)cc(Cn4ccnc4NC)cc3C2=O)ncc1F] (S)-2-(1-(4-ethoxy-5-fluoropyridin-2- yl)ethyl)-7-((2-(methylamino)-1H- imidazol-1-yl)methyl)-5- (morpholinomethyl)-3,4- dihydroisoquinolin-1(2H)-one | ¹H NMR (400 MHz, Methanol-d₄) δ ppm 8.24 (d, J = 3.3 Hz, 1H), 7.78 (d, J = 1.6 Hz, 1H), 7.23 (d, J = 1.7 Hz, 1H), 7.13 (d, J = 6.9 Hz, 1H), 6.61 (dd, J = 11.2, 1.7 Hz, 2H), 5.97 (q, J = 7.1 Hz, 1H), 4.96 (s, 2H), 4.19 (qd, J = 7.0, 3.4 Hz, 2H), 3.67 -3.58 (m, 4H), 3.53 (ddd, J = 13.8, 9.2, 4.8 Hz, 1H), 3.48 (s, 2H), 3.40 - 3.34 (m, 1H), 3.02 (dd, J = 7.2, 5.0 Hz, 1H), 2.94 (dd, J = 9.3, 5.2 Hz, 1H), 2.84 (s, 3H), 2.38 (s, 4H), 1.62 (d, J = 7.1 Hz, 3H), 1.43 (t, J = 7.0 Hz, 3H). LC-MS: (ESI) m/z 523.3 [M + H]⁺ | Step 2: intermediate (A1) and intermediate (G5) Step 3: potassium trifluoro(morpholin omethyl)borate |
| 28 | 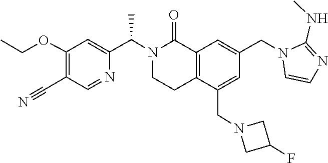 [SMILES: CCOc1cc([C@H](C)N2CCc3c(CN4CC(F)C4)cc(Cn4ccnc4NC)cc3C2=O)ncc1C#N] (S)-4-ethoxy-6-(1-(5-((3- fluoroazetidin-1-yl)methyl)-7-((2- (methylamino)-1H-imidazol-1- yl)methyl)-1-oxo-3,4- dihydroisoquinolin-2(1H)- yl)ethyl)nicotinonitrile | ¹H NMR (400 MHz, Methanol-d₄) δ ppm 7.16 (s, 1H), 6.31 (d, J = 1.6 Hz, 1H), 5.75 (d, J = 1.6 Hz, 1H), 5.70 (s, 1H), 5.14 (dd, J = 13.3, 1.7 Hz, 2H), 4.49 (q, J = 7.1 Hz, 1H), 3.64 (dt, J = 57.5, 5.2 Hz, 1H), 3.50 (s, 2H), 2.83 (q, J = 7.0 Hz, 2H), 2.22 (s, 2H), 2.12 (ddd, J = 15.9, 6.7, 3.2 Hz, 5H), 1.80 (s, 1H), 1.73 (dd, J = 9.5, 4.6 Hz, 1H), 1.57 - 1.42 (m, 2H), 1.38 (s, 3H), 0.19 (d, J = 7.1 Hz, 3H), - 0.00 (t, J = 7.0 Hz, 3H). LC-MS: (ESI) m/z 518.2 [M + H]⁺ | Step 2: intermediate (A1) and intermediate (G1) Step 3: potassium trifluoro(morpholin omethyl)borate was replace with potassium trifluoro((3- fluoroazetidin-1- yl)methyl)borate |
| 29 | 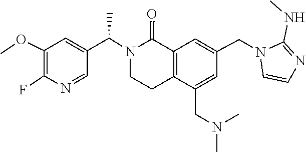 [SMILES: CNc1nccn1Cc1cc(CN(C)C)c2c(c1)C(=O)N([C@@H](C)c1cnc(F)c(OC)c1)CC2] (S)-5-((dimethylamino)methyl)-2-(1- (6-fluoro-5-methoxypyridin-3- yl)ethyl)-7-((2-(methylamino)-1H- imidazol-1-yl)methyl)-3,4- dihydroisoquinolin-1(2H)-one | ¹H NMR (400 MHz, Methanol-d₄) δ ppm 7.83 (d, J = 1.4 Hz, 1H), 7.73 (s, 1H), 7.48 (dd, J = 9.7, 1.8 Hz, 1H), 7.23 (d, J = 1.6 Hz, 1H), 6.63 (d, J = 1.7 Hz, 1H), 6.59 (d, J = 1.7 Hz, 1H), 6.07(q, J = 7.1 Hz, 1H), 4.97 (s, 2H), 3.89 (s, 3H), 3.51 (ddd, J = 16.0, 8.1, 3.3 Hz, 1H), 3.42 (s, 2H), 3.22 (ddd, J = 12.4, 6.9, 5.2 Hz, 1H), 3.03 (ddd, J = 16.3, 6.8, 4.8 Hz, 1H), 2.84 (m, 4H), 2.20 (s, 6H), 1.65 (d, J = 7.1 Hz, 3H). LC-MS: (ESI) m/z 466.9 [M + H]⁺ | Step 2: intermediate (A1) and intermediate (G7) Step 3: potassium trifluoro(morpholin omethyl)borate was replaced with potassium ((dimethylamino)m ethyl)trifluoroborat e |
| 30 | 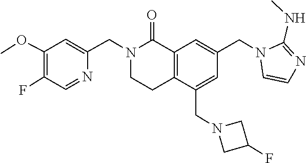 [SMILES: CNc1nccn1Cc1cc(CN2CC(F)C2)c2c(c1)C(=O)N(Cc1cc(OC)c(F)cn1)CC2] 2-((5-fluoro-4-methoxypyridin-2- yl)methyl)-5-((3-fluoroazetidin-1- yl)methyl)-7-((2-(methylamino)-1H- imidazol-1-yl)methyl)-3,4- dihydroisoquinolin-1(2H)-one | ¹H NMR (400 MHz, Methanol-d₄) δ ppm 8.21 (d, J = 3.4 Hz, 1H), 7.77 (d, J = 1.5 Hz, 1H), 7.35 (d, J = 1.5 Hz, 1H), 7.17 (d, J = 7.0 Hz, 1H), 6.94 (d, J = 2.4 Hz, 1H), 6.90 (d, J = 2.4 Hz, 1H), 5.09 (s, 3H), 4.81 (s, 2H), 3.95 (s, 3H), 3.74 (s, 2H), 3.68 - 3.56 (m, 4H), 3.06 (t, J = 6.7 Hz, 2H), 2.98 (s, 3H). LC-MS: (ESI) m/z 483.2 [M + H]⁺ | Step 2: intermediate (A1) and intermediate (G10) Step 3: potassium trifluoro(morpholin omethyl)borate was replaced with potassium trifluoro((3- fluoroazetidin-1- yl)methyl)borate |
| 31 | 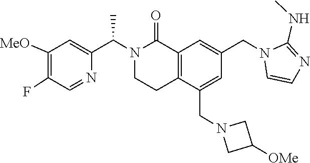 [SMILES: CNc1nccn1Cc1cc(CN2CC(OC)C2)c2c(c1)C(=O)N([C@@H](C)c1cc(OC)c(F)cn1)CC2] (S)-2-(1-(5-fluoro-4-methoxypyridin- 2-yl)ethyl)-5-((3-methoxyazetidin-1- yl)methyl)-7-((2-(methylamino)-1H- imidazol-1-yl)methyl)-3,4- dihydroisoquinolin-1(2H)-one | ¹H NMR (400 MHz, Methanol-d₄) δ ppm 8.23 (d, J = 3.2 Hz, 1H), 7.78 (s, 1H), 7.23 - 7.07 (m, 2H), 6.61 (dd, J = 14.1, 1.6 Hz, 2H), 5.97 (q, J = 7.1 Hz, 1H), 4.96 (s, 2H), 4.05 -3.95 (m, 1H), 3.94 (s, 3H), 3.64 (s, 2H), 3.58 - 3.46 (m, 3H), 3.37 (dd, J = 13.5, 6.3 Hz, 1H), 3.01 -2.95 (m, 2H), 2.84 (s, 5H), 1.62 (d, J = 7.1 Hz, 3H). LC-MS: (ESI) m/z 509.3 [M + H]⁺ | Step 2: intermediate (A1) and intermediate (G9) Step 3: potassium trifluoro(morpholin omethyl)borate was replaced with potassium trifluoro((3- methoxyazetidin- l-yl)methyl)borate |
| 32 | 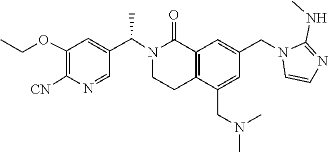 (S)-5-(1-(5-((dimethylamino)methyl)- 7-((2-(methylamino)-1H-imidazol-1- yl)methyl)-1-oxo-3,4- dihydroisoquinolin-2(1H)-yl)ethyl)-3- ethoxypicolinonitrile | 1H NMR (400 MHz, Methanol-d₄) δ ppm 8.27 (d, J = 1.4 Hz, 1H), 7.81 (d, J = 1.4 Hz, 1H), 7.58 (s, 1H), 7.24 (d, J = 1.5 Hz, 1H), 6.61 (dd, J = 14.5, 1.7 Hz, 2H), 6.06 (q' J = 7.1 Hz, 1H), 4.97 (s, 2H), 4.26 (qd, J = 7.0, 2.0 Hz, 2H), 3.55 (ddd, J = 13.9, 9.5, 4.7 Hz, 1H), 3.43 (s, 2H), 3.08 - 3.00 (m, 1H), 2.94 (dd, J = 9.5, 5.3 Hz, 1H), 2.84 (s, 3H), 2.20 (s, 6H), 1.68 (d, J = 7.2 Hz, 3H), 1.46 (t, J = 7.0 Hz, 3H). LC-MS: (ESI) m/z 487.9 [M + H]⁺ | Step 2: intermediate (A1) and intermediate (G3) Step 3: potassium trifluoro(morpholin omethyl)borate was replaced with potassium ((dimethylamino)m ethyl)trifluoroborat e |
| 33 | 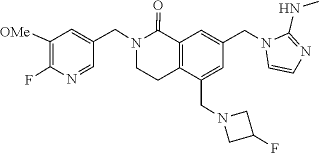 [SMILES: CNc1nccn1Cc1cc(CN2CC(F)C2)c2c(c1)C(=O)N(Cc1cnc(F)c(OC)c1)CC2] 2-((6-fluoro-5-methoxypyridin-3- yl)methyl)-5-((3-fluoroazetidin-1- yl)methyl)-7-((2-(methylamino)-1H- imidazol-1-yl)methyl)-3,4- dihydroisoquinolin-1(2H)-one | ¹H NMR (400 MHz, Methanol-d₄) δ ppm 7.95 (d, J = 1.3 Hz, 1H), 7.69 (s, 1H), 7.62 - 7.46 (m, 2H), 7.01 (d, J = 2.5 Hz, 1H), 6.94 (d, J = 2.5 Hz, 1H), 5.42 (d, J = 56.7 Hz, 1H), 5.15 (s, 2H), 4.78 (s, 2H), 4.58 (s, 4H), 4.37 (ddd, J = 20.5, 12.9, 3.2 Hz, 2H), 3.89 (s, 3H), 3.66 (t, J = 6.6 Hz, 2H), 3.11 (t, J = 6.6 Hz, 2H), 3.00 (s, 3H). LC-MS: (ESI) m/z 483.0 [M + H]⁺ | Step 2: intermediate (A1) and intermediate (G8) Step 3: potassium trifluoro(morpholin omethyl)borate was replaced with potassium trifluoro((3- fluoroazetidin-1- yl)methyl)borate |
| 34 | 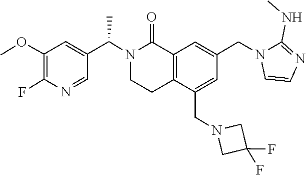 [SMILES: CNc1nccn1Cc1cc(CN2CC(F)(F)C2)c2c(c1)C(=O)N([C@@H](C)c1cnc(F)c(OC)c1)CC2] (S)-5-((3,3-difluoroazetidin-1- yl)methyl)-2-(1-(6-fluoro-5- methoxypyridin-3-yl)ethyl)-7-((2- (methylamino)-1H-imidazol-1- yl)methyl)-3,4-dihydroisoquinolin- 1(2H)-one | ¹H NMR (400 MHz, Methanol-d₄) δ ppm 7.82 (s, 1H), 7.73 (s, 1H), 7.48 (dd, J = 9.7, 1.8 Hz, 1H), 7.28 - 7.20 (m, 1H), 6.63 (d, J = 1.7 Hz, 1H), 6.59 (d, J = 1.7 Hz, 1H), 6.07 (q, J = 7.1 Hz, 1H), 4.97 (s, 2H), 3.89 (s, 3H), 3.82 - 3.67 (m, 2H), 3.62 - 3.44 (m, 5H), 3.22 (ddd, J = 12.5, 6.9, 5.3 Hz, 1H), 3.06 - 2.92 (m, 1H), 2.84 (s, 4H), 1.65 (d, J = 7.1 Hz, 3H). LC-MS: (ESI) m/z 514.9 [M + H]⁺ | Step 2: intermediate (A1) and intermediate (G7) Step 3: potassium trifluoro(morpholin omethyl)borate was replaced with potassium ((3,3- difluoroazetidin-1- yl)methyl)trifluorob orate |
| 35 | 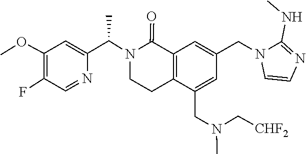 [SMILES: CNc1nccn1Cc1cc(CN(C)CC(F)F)c2c(c1)C(=O)N([C@@H](C)c1cc(OC)c(F)cn1)CC2] (S)-5-(((2,2- difluoroethyl)(methyl)amino)methyl)- 2-(1-(5-fluoro-4-methoxypyridin-2- yl)ethyl)-7-((2-(methylamino)-1H- imidazol-1-yl)methyl)-3,4- dihydroisoquinolin-1(2H)-one | ¹H NMR (400 MHz, Methanol-d₄) δ ppm 8.24 (d, J = 3.2 Hz, 1H), 7.79 (s, 1H), 7.22 (s, 1H), 7.16 (d, J = 7.0 Hz, 1H), 6.61 (d, J = 8.7 Hz, 2H), 5.97 (d, J = 6.7 Hz, 1H), 4.96 (d, J = 5.3 Hz, 2H), 3.93 (s, 3H), 3.60 (s, 2H), 3.51 (dd, J = 8.8, 4.4 Hz, 1H), 3.40 - 3.34 (m, 1H), 3.07 - 2.96 (m, 1H), 2.94 (dd, J = 9.4, 5.4 Hz, 1H), 2.84 (s, 3H), 2.70 (td, J = 15.1, 4.1 Hz, 2H), 2.26 (s, 3H), 1.62 (d, J = 7.1 Hz, 3H). LC-MS: (ESI) m/z 517.3 [M + H]⁺ | Step 2: intermediate (A1) and intermediate (G9) Step 3: potassium trifluoro(morpholin omethyl)borate was replaced with potassium (((2,2- difluoroethyl)(meth yl)amino)methyl)tri fluoroborate |
| 36 | 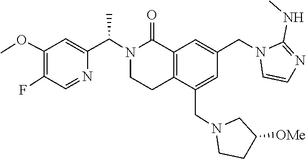 [SMILES: CNc1nccn1Cc1cc(CN2CC[C@@H](OC)C2)c2c(c1)C(=O)N([C@@H](C)c1cc(OC)c(F)cn1)CC2] 2-((S)-1-(5-fluoro-4-methoxypyridin- 2-yl)ethyl)-5-(((R)-3- methoxypyrrolidin-1-yl)methyl)-7-((2- (methylamino)-1H-imidazol-1- yl)methyl)-3,4-dihydroisoquinolin- 1(2H)-one | ¹H NMR (400 MHz, Methanol-d₄) δ ppm 8.23 (d, J = 3.2 Hz, 1H), 7.79 (s, 1H), 7.25 (s, 1H), 7.15 (d, J = 6.9 Hz, 1H), 6.59 (dd, J = 13.5, 1.6 Hz, 2H), 5.97 (q, J = 7.0 Hz, 1H), 4.95 (s, 2H), 3.93 (s, 4H), 3.64 -3.55 (m, 2H), 3.52 (dt, J = 9.2, 4.3 Hz, 1H), 3.37 (ddd, J = 13.0, 7.2, 5.5 Hz, 1H), 3.08- 2.97 (m, 1H), 2.92 (dd, J = 9.3, 5.2 Hz, 1H), 2.84 (s, 3H), 2.64 (dq, J = 14.0, 7.8, 7.1 Hz, 2H), 2.53 (dd, J = 10.3, 3.1 Hz, 1H), 2.41 (q, J = 8.0 Hz, 1H), 2.04 (dd, J = 13.6, 6.2 Hz, 1H), 1.73 (dd, J = 8.3, 5.2 Hz, 1H), 1.62 (d, J = 7.1 Hz, 3H), 0.88 (d, J = 7.8 Hz, 1H). LC-MS: (ESI) m/z 523.3 [M + H]⁺ | Step 2: intermediate (A1) and intermediate (G9) Step 3: potassium trifluoro(morpholin omethyl)borate was replaced with potassium (R)- trifluoro((3- methoxypyrrolidin- 1-yl)methyl)borate |
| 37 | 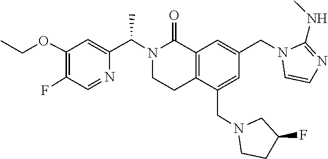 [SMILES: CCOc1cc([C@H](C)N2CCc3c(CN4CC[C@H](F)C4)cc(Cn4ccnc4NC)cc3C2=O)ncc1F] 2-((S)-1-(4-ethoxy-5-fluoropyridin-2- yl)ethyl)-5-(((S)-3-fluoropyrrolidin-1- yl)methyl)-7-((2-(methylamino)-1H- imidazol-1-yl)methyl)-3,4- dihydroisoquinolin-1(2H)-one | ¹H NMR (400 MHz, Methanol-d₄) δ ppm 8.26 (d, J = 3.3 Hz, 1H), 7.82 (d, J = 1.9 Hz, 1H), 7.29 (d, J = 1.9 Hz, 1H), 7.15 (d, J = 6.9 Hz, 1H), 6.63 (dd, J = 12.7, 1.7 Hz, 2H), 5.99 (q, J = 7.1 Hz, 1H), 5.26 Step 3: -5.04 (m, 1H), 4.99 (s, 2H), 4.21 (qd, J = 7.0, 3.5 Hz, 2H), 3.74- 3.51 (m, 3H), 3.39 (ddd, J = 12.7, 7.1, 5.3 Hz, 1H), 3.13 -2.90 (m, 2H), 2.89 - 2.57 (m, 6H), 2.44 - 2.33 (m, 1H), 2.17 (ddq, J = 27.2, 13.8, 6.6 Hz, 1H), 1.96 (ddt, J = 29.5, 14.2, 7.0 Hz, 1H), 1.64 (d, J = 7.1 Hz, 3H), 1.45 (t, J = 7.0 Hz, 3H). LC-MS: (ESI) m/z 525.3 [M + H]⁺ | Step 2: intermediate (A1) and intermediate (G5) potassium trifluoro(morpholin omethyl)borate was replaced with potassium (5)- trifluoro((3- fluoropyrrolidin-1- yl)methyl)borate |
| 38 | 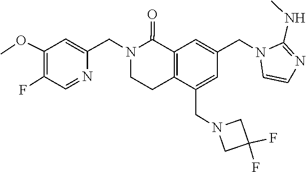 [SMILES: CNc1nccn1Cc1cc(CN2CC(F)(F)C2)c2c(c1)C(=O)N(Cc1cc(OC)c(F)cn1)CC2] 5-((3,3-difluoroazetidin-1-yl)methyl)- 2-((5-fluoro-4-methoxypyridin-2- yl)methyl)-7-((2-(methylamino)-1H- imidazol-1-yl)methyl)-3,4- dihydroisoquinolin-1(2H)-one | ¹H NMR (400 MHz, Methanol-d₄) δ ppm 8.19 (d, J = 3.4 Hz, 1H), 7.77 (d, J = 1.9 Hz, 1H), 7.22 (d, J = 1.9 Hz, 1H), 7.14 (d, J = 7.0 Hz, 1H), 6.58 (dd, J = 16.0, 1.7 Hz, 2H), 4.94 (s, 2H), 4.77 (s, 2H), 3.91 (s, 3H), 3.74 (s, 2H), 3.66 - 3.49 (m, 6H), 3.03 (t, J = 6.7 Hz, 2H), 2.81 (s, 3H). LC-MS: (ESI) m/z 501.2 [M + H]⁺ | Step 2: intermediate (A1) and intermediate (G10) Step 3: potassium trifluoro(morpholin omethyl)borate was replaced with potassium ((3,3- difluoroazetidin-1- yl)methyl)trifluorob orate |
| 39 | 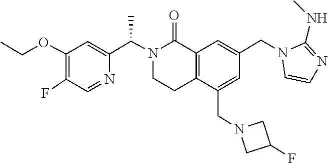 [SMILES: CCOc1cc([C@H](C)N2CCc3c(CN4CC(F)C4)cc(Cn4ccnc4NC)cc3C2=O)ncc1F] (S)-2-(1-(4-ethoxy-5-fluoropyridin-2- yl)ethyl)-5-((3-fluoroazetidin-1- yl)methyl)-7-((2-(methylamino)-1H- imidazol-1-yl)methyl)-3,4- dihydroisoquinolin-1(2H)-one | ¹H NMR (400 MHz, Methanol-d₄) δ ppm 8.23 (d, J = 3.3 Hz, 1H), 7.79 (d, J = 1.4 Hz, 1H), 7.20 (d, J = 1.5 Hz, 1H), 7.13 (d, J = 6.9 Hz, (G5) 1H), 6.63 (d, J = 1.7 Hz, 1H), 6.59 (d, J = 1.7 Hz, 1H), 5.96 (q, J = 7.1 Hz, 1H), 5.09 (dt, J = 58.0, 5.2 Hz, 1H), 4.96 (s, 2H), 4.19 (qd, J = 7.0, 2.9 Hz, 2H), 3.67 (s, 2H), 3.55 (dtt, J = 13.8, 9.4, 5.3 Hz, 3H), 3.41 - 3.33 (m, 1H), 3.22 (ddd, J = 23.2, 9.4, 4.6 Hz, 2H), 3.02 - 2.76 (m, 5H), 1.61 (d, J = 7.1 Hz, 3H), 1.42 (t, J = 7.0 Hz, 3H). LC-MS: (ESI) m/z 511.3 [M + H]⁺ | Step 2: intermediate (A1) and intermediate Step 3: potassium trifluoro(morpholin omethyl)borate was replaced with potassium trifluoro((3- fluoroazetidin-1- yl)methyl)borate |
| 40 | 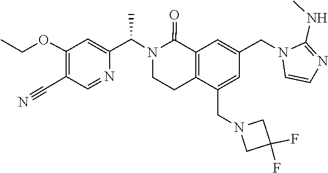 [SMILES: CCOc1cc([C@H](C)N2CCc3c(CN4CC(F)(F)C4)cc(Cn4ccnc4NC)cc3C2=O)ncc1C#N] (S)-6-(1-(5-((3,3-difluoroazetidin-1- yl)methyl)-7-((2-(methylamino)-1H- imidazol-1-yl)methyl)-1-oxo-3,4- dihydroisoquinolin-2(1H)-yl)ethyl)-4- ethoxynicotinonitrile | 1H NMR (400 MHz, Methanol-d₄) δ ppm 8.63 (s, 1H), 7.77 (d, J = 1.9 Hz, 1H), 7.29 - 7.13 (m, 2H), 6.62 (dd, J = 12.2, 1.7 Hz, 2H), 5.95 (q, J = 7.1 Hz, 1H), 4.97 (s, 2H), 4.36 -4.23 (m, 2H), 3.76 (s, 2H), 3.64 -3.44 (m, 6H), 2.98 (dtt, J = 20.1, 10.7, 4.7 Hz, 2H), 2.85 (s, 3H), 1.65 (d, J = 7.1 Hz, 3H), 1.46 (t, J = 7.0 Hz, 3H). LC-MS: (ESI) m/z 536.3 [M + H]⁺ | Step 2: intermediate (A1) and intermediate (G1) Step 3: potassium trifluoro(morpholin omethyl)borate was replaced with potassium ((3,3- difluoroazetidin-1- yl)methyl)trifluorob orate |
| 41 | 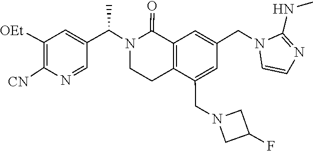 (S)-3-ethoxy-5-(1-(5-((3- fluoroazetidin-1-yl)methyl)-7-((2- (methylamino)-1H-imidazol-1- yl)methyl)-1-oxo-3,4- dihydroisoquinolin-2(1H)- yl)ethyl)picolinonitrile | ¹H NMR (400 MHz, Methanol-d₄) δ ppm 8.35 - 8.22 (m, 1H), 7.80 (s, 1H), 7.58 (t, J = 1.2 Hz, 1H), 7.24 (d, J = 8.6 Hz, 1H), 6.64 (dd, J = 11.6, 1.8 Hz, 2H), 6.07 (q, J = 7.1 Hz, 1H), 4.98 (s, 2H), 4.26 (qd, J = 7.0, 2.3 Hz, 2H), 3.65- 3.47 (m, 4H), 3.29 - 3.10 (m, 4H), 3.01 (dt, J = 16.5, 5.9 Hz, 1H), 2.86 (s, 4H), 2.03 (d, J = 6.0 Hz, 1H), 1.68 (d, J = 7.1 Hz, 3H), 1.46 (d, J = 7.0 Hz, 3H). LC-MS: (ESI) m/z 518.3 [M + H]⁺ | Step 2: intermediate (A1) and intermediate (G3) Step 3: potassium trifluoro(morpholin omethyl)borate was replaced with potassium trifluoro((3- fluoroazetidin-1- yl)methyl)borate |
| 42 | 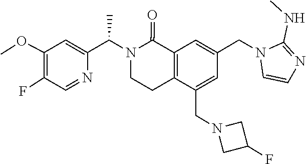 [SMILES: CNc1nccn1Cc1cc(CN2CC(F)C2)c2c(c1)C(=O)N([C@@H](C)c1cc(OC)c(F)cn1)CC2] (S)-2-(1-(5-fluoro-4-methoxypyridin- 2-ypethyl)-5-((3-fluoroazetidin-1- yl)methyl)-7-((2-(methylamino)-1H- imidazol-1-yl)methyl)-3,4- dihydroisoquinolin-1(2H)-one | ¹H NMR (400 MHz, Methanol-d₄) δ ppm 6.61 (d, J = 3.2 Hz, 1H), 6.16 (d, J = 1.9 Hz, 1H), 5.66- 5.47 (m, 2H), 5.00 (dd, J = 12.0, 1.8 Hz, 2H), 4.34 (q, J = 7.0 Hz, 1H), 3.54 (q, J = 5.1 Hz, 1H), 3.34 (s, 2H), 2.31 (s, 3H), 2.05 (s, 2H), 2.00 - 1.84 (m, 3H), 1.76 (q, J = 5.8 Hz, 1H), 1.64 - 1.48 (m, 2H), 1.42- 1.16 (m, 5H), -0.01 (d, J = 7.1 Hz, 3H). LC-MS: (ESI) m/z 497.3 [M + H]⁺ | Step 2: intermediate (A1) and intermediate (G9) Step 3: potassium trifluoro(morpholin omethyl)borate was replaced with potassium trifluoro((3- fluoroazetidin-1- yl)methyl)borate |
| 43 | 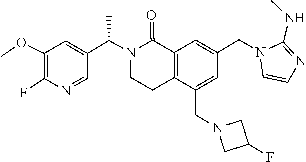 [SMILES: CNc1nccn1Cc1cc(CN2CC(F)C2)c2c(c1)C(=O)N([C@@H](C)c1cnc(F)c(OC)c1)CC2] (S)-2-(1-(6-fluoro-5-methoxypyridin- 3-yl)ethyl)-5-((3-fluoroazetidin-1- yl)methyl)-7-((2-(methylamino)-1H- imidazol-1-yl)methyl)-3,4- dihydroisoquinolin-1(2H)-one | ¹H NMR (400 MHz, Methanol-d₄) δ ppm 7.82 (d, J = 1.5 Hz, 1H), 7.73 (s, 1H), 7.48 (dd, J = 9.8, 1.9 Hz, 1H), 7.21 (d, J = 1.5 Hz, 1H), 6.63 (d, J = 1.7 Hz, 1H), 6.59 (d, J = 1.7 Hz, 1H), 6.08 (t, J = 7.0 Hz, 1H), 5.09 (dt, J = 58.0, 5.3 Hz, 1H), 4.97 (s, 2H), 3.89 (s, 3H), 3.67 (s, 2H), 3.54 (dtd, J = 20.5, 9.3, 8.9, 3.6 Hz, 3H), 3.27 - 3.16 (m, 3H), 3.04 - 2.92 (m, 1H), 2.84 (s, 4H), 1.65 (d, J = 7.1 Hz, 3H). LC-MS: (ESI) m/z 497.3 [M + H]⁺ | Step 2: intermediate (A1) and intermediate (G7) Step 3: potassium trifluoro(morpholin omethyl)borate was replaced with potassium trifluoro((3- fluoroazetidin-1- yl)methyl)borate |
| 44 | 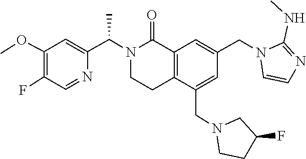 [SMILES: CNc1nccn1Cc1cc(CN2CC[C@H](F)C2)c2c(c1)C(=O)N([C@@H](C)c1cc(OC)c(F)cn1)CC2] 2-((S)-1-(5-fluoro-4-methoxypyridin- 2-yl)ethyl)-5-(((S)-3-fluoropyrrolidin- 1-yl)methyl)-7-((2-(methylamino)-1H- imidazol-1-yl)methyl)-3,4- dihydroisoquinolin-1(2H)-one | ¹H NMR (400 MHz, Methanol-d₄) δ ppm 8.27 (d, J = 3.3 Hz, 1H), 7.83 (d, J = 1.8 Hz, 1H), 7.30 (d, J = 1.9 Hz, 1H), 7.19 (d, J = 6.9 Hz, 1H), 6.64 (dd, J = 12.1, 1.7 Hz, 2H), 6.01 (q, J = 7.1 Hz, 1H), 5.24 -5.05 (m, 1H), 5.00 (s, 2H), 3.97 (5, 3H), 3.72 - 3.41 (m, 4H), 3.12 -2.94 (m, 2H), 2.88 (s, 3H), 2.81 (dt, J = 17.1, 8.8 Hz, 2H), 2.66 (ddd, J = 30.1, 11.6, 5.0 Hz, 1H), 2.40 (q, J = 7.9 Hz, 1H), 2.19 (dt, J = 20.3, 7.2 Hz, 1H), 2.07 - 1.94 (m, 1H), 1.66 (d, J = 7.1 Hz, 3H). LC-MS: (ESI) m/z 511.3 [M + H]⁺ | Step 2: intermediate (A1) and intermediate (G9) Step 3: potassium trifluoro(morpholin omethyl)borate was replaced with potassium (5)- trifluoro((3- fluoropyrrolidin-1- yl)methyl)borate |
| 45 | 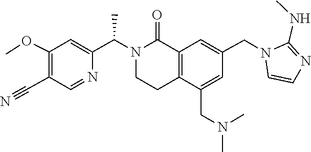 [SMILES: CNc1nccn1Cc1cc(CN(C)C)c2c(c1)C(=O)N([C@@H](C)c1cc(OC)c(C#N)cn1)CC2] (S)-6-(1-(5-((dimethylamino)methyl)- 7-((2-(methylamino)-1H-imidazol-1- yl)methyl)-1-oxo-3,4- dihydroisoquinolin-2(1H)-yl)ethyl)-4- methoxynicotinonitrile | ¹H NMR (400 MHz, Methanol-d₄) δ ppm 8.63 ppm (s, 1H), 7.78 (d, J = 1.6 Hz, 1H), 7.22 (d, J = 12.8 Hz, 2H), 6.61 (dd, J = 13.4, 1.7 Hz, 2H), 5.97 (q, J = 7.1 Hz, 1H), 4.96 (s, 2H), 4.03 (s, 3H), 3.60 (ddd, J = 13.9, 9.1, 5.0 Hz, 1H), 3.55 - 3.46 (m, 1H), 3.44 (s, 2H), 3.10- 2.92 (m, 2H), 2.84 (s, 3H), 2.21 (s, 6H), 1.66 (d, J = 7.1 Hz, 3H). LC-MS: (ESI) m/z 474.3 [M + H]⁺ | Step 2: intermediate (A1) and intermediate (G12) Step 3: potassium trifluoro(morpholin omethyl)borate was replaced with potassium ((dimethylamino)m ethyl)trifluoroborate |
| 46 | 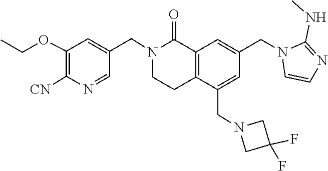 5-((5((3,3-difluoroazetidin-1- yl)methyl)-7-((2-(methylamino)-1H- imidazol-1-yl)methyl)-1-oxo-3,4- dihydroisoquinolin-2(1H)-yl)methyl)- 3-ethoxypicolinonitrile | ¹H NMR (400 MHz, Methanol-d₄) δ ppm 8.47 (s, 1H), 8.20 (d, J = 1.6 Hz, 1H), 7.76 (d, J = 1.9 Hz, 1H), 7.59 (d, J = 1.7 Hz, 1H), 7.35 (d, J = 2.0 Hz, 1H), 6.91 -6.80 (m, 2H), 5.04 (s, 2H), 4.82 (s, 2H), 4.22 (q, J = 7.0 Hz, 2H), 3.77 (s, 2H), 3.65 - 3.52 (m, 6H), 3.06 (t, J = 6.7 Hz, 2H), 2.93 (s, 3H), 1.43 (t, J = 7.0 Hz, 3H). LC-MS: (ESI) m/z 522.2 [M + H]⁺ | Step 2: intermediate (A1) and intermediate (G4) Step 3: potassium trifluoro(morpholin omethyl)borate was replaced with potassium ((3,3- difluoroazetidin-1- yl)methyl)trifluorob orate |
| 47 | 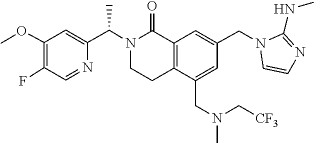 (S)-2-(1-(5-fluoro-4-methoxypyridin- 2-yl)ethyl)-5-((methyl(2,2,2- trifluoroethyl)amino)methyl)-7-((2- (methylamino)-1H-imidazol-1- yl)methyl)-3,4-dihydroisoquinolin- 1(2H)-one | ¹H NMR (400 MHz, Methanol-d₄) δ ppm 8.24 (d, J = 3.3 Hz, 1H), 7.80 (d, J = 1.9 Hz, 1H), 7.24- 7.09 (m, 2H), 6.60 (dd, J = 10.3, 1.7 Hz, 2H), 5.98 (q, J = 7.1 Hz, 1H), 4.97 (s, 2H), 3.94 (s, 3H), 3.69 (s, 2H), 3.58 - 3.47 (m, 1H), 3.42 - 3.33 (m, 1H), 3.10 - 2.86 (m, 4H), 2.84 (s, 3H), 2.33 (s, 3H), 1.63 (d, J = 7.1 Hz, 3H). LC-MS: (ESI) m/z 535.2 [M + H]⁺ | Step 2: intermediate (A1) and intermediate (G9) Step 3: potassium trifluoro(morpholin omethyl)borate was replaced with potassium trifluoro((methyl(2, 2,2- trifluoroethyl)amin o)methyl)borate |
| 48 | 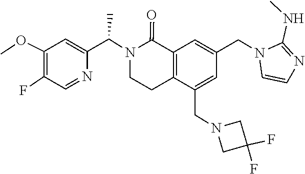 [SMILES: CNc1nccn1Cc1cc(CN2CC(F)(F)C2)c2c(c1)C(=O)N([C@@H](C)c1cc(OC)c(F)cn1)CC2] (S)-5-((3,3-difluoroazetidin-1- yl)methyl)-2-(1-(5-fluoro-4- methoxypyridin-2-yl)ethyl)-7-((2- (methylamino)-1H-imidazol-1- yl)methyl)-3,4-dihydroisoquinolin- 1(2H)-one | ¹H NMR (400 MHz, Methanol-d₄) δ ppm 8.24 (d, J = 3.3 Hz, 1H), 7.79 (d, J = 1.9 Hz, 1H), 7.26 (d, J = 1.9 Hz, 1H), 7.16 (d, J = 6.9 Hz, 1H), 6.70 -6.63 (m, 2H), 5.97 (q, J = 7.1 Hz, 1H), 4.99 (s, 2H), 3.94 (s, 3H), 3.75 (d, J = 2.0 Hz, 2H), 3.64 -3.50 (m, 5H), 3.39 (ddd, J = 12.6, 7.1, 5.2 Hz, 1H), 2.99 (ddd, J = 16.5, 7.1, 4.9 Hz, 1H), 2.87 (s, 4H), 1.62 (d, J = 7.1 Hz, 3H). LC-MS: (ESI) m/z 515.2 [M + H]⁺ | Step 2: intermediate (A1) and intermediate (G9) Step 3: potassium trifluoro(morpholin omethyl)borate was replaced with potassium ((3,3- difluoroazetidin-1- yl)methyl)trifluorob orate |
| 49 | 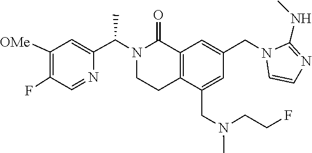 [SMILES: CNc1nccn1Cc1cc(CN(C)CCF)c2c(c1)C(=O)N([C@@H](C)c1cc(OC)c(F)cn1)CC2] (S)-2-(1-(5-fluoro-4-methoxypyridin- 2-yl)ethyl)-5-(((2- fluoroethyl)(methyl)amino)methyl)-7- ((2-(methylamino)-1H-imidazol-1- yl)methyl)-3,4-dihydroisoquinolin- 1(2H)-one | 1H NMR (400 MHz, Methanol-d₄) δ ppm 8.21 (d, J = 3.3 Hz, 1H), 7.79 - 7.72 (m, 1H), 7.25 (d, J = 1.9 Hz, 1H), 7.13 (d, J = 6.9 Hz, 1H), 6.70 -6.59 (m, 2H), 5.95 (q, J = 7.1 Hz, 1H), 4.96 (s, 2H), 4.52 Step 3: (t, J = 4.9 Hz, 1H), 4.40 (t, J = 4.9 Hz, 1H), 3.91 (s, 3H), 3.56 - 3.45 (m, 3H), 3.40 - 3.32 (m, 1H), 3.03 (dt, J = 16.5, 5.9 Hz, 1H), 2.84 (s, was 4H), 2.65 (dt, J = 27.8, 4.8 Hz, 2H), 2.19(s, 3H), 1.60 (d, J = 7.1 Hz, 3H). LC-MS: (ESI) m/z 499.3 [M + H]⁺ | Step 2: intermediate (A1) and intermediate (G9) potassium trifluoro(morpholin omethyl)borate replaced with potassium trifluoro(((2- fluoroethyl)(methyl) amino)methyl)bor ate |
| 50 | 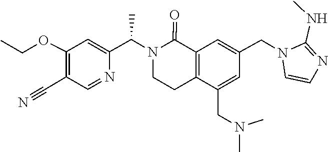 [SMILES: CCOc1cc([C@H](C)N2CCc3c(CN(C)C)cc(Cn4ccnc4NC)cc3C2=O)ncc1C#N] (S)-6-(1-(5-((dimethylamino)methyl)- 7-((2-(methylamino)-1H-imidazol-1- yl)methyl)-1-oxo-3,4- dihydroisoquinolin-2(1H)-yl)ethyl)-4- ethoxynicotinonitrile | ¹H NMR (400 MHz, Methanol-d₄) δ 8.63 (s, 1H), 7.78 (d, J = 1.9 Hz, 1H), 7.23 (d, J = 2.0 Hz, 1H), 7.17 (s, 1H), 6.60 (dd, J = 14.2, 1.7 Hz, 2H), 5.95 (q, J = 7.1 Hz, 1H), 4.96 (s, 2H), 4.40 -4.22 (m, 2H), 3.63 - 3.55 (m, 1H), 3.55 -3.46 (m, 1H), 3.44(s, 2H), 3.17 - 2.91 (m, 2H), 2.83 (s, 3H), 2.21 (s, 6H), 1.65 (d, J = 7.1 Hz, 3H), 1.46 (t, J = 7.0 Hz, 3H). LC-MS: (ESI) m/z 488.3 [M + H]⁺ | Step 2: intermediate (A1) and intermediate (G1) Step 3: potassium trifluoro(morpholin omethyl)borate was replaced with potassium ((dimethylamino)m ethyl)trifluoroborate |

### Example 51: Synthesis of (S)-2-(1-(5-fluoro-4-methoxypyridin-2-yl)ethyl)-5-((3-hydroxyazetidin-1-yl)methyl)-7-((2-(methylamino)-1H-imidazol-1-yl)methyl)-3,4-dihydroisoquinolin-1(2H)-one (51)

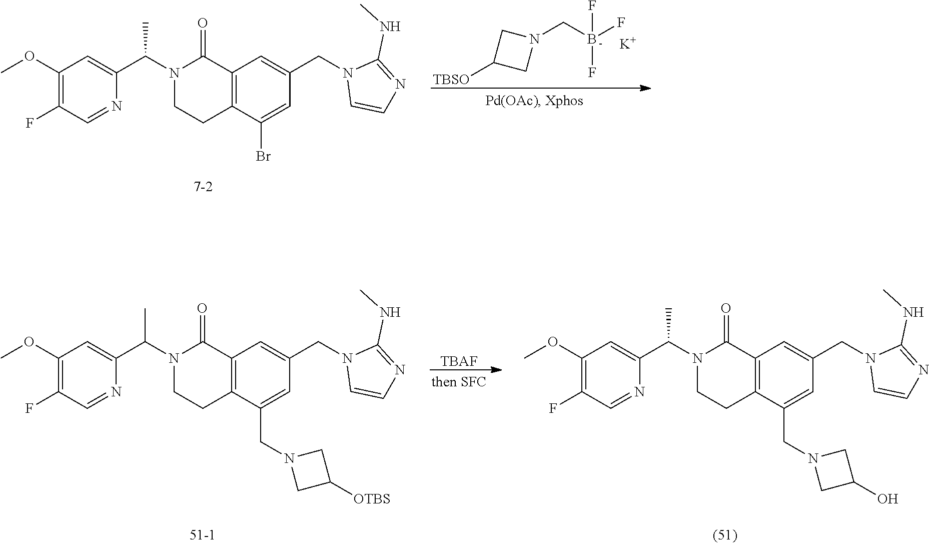

Step 1: Compound (7-2) (30 mg, 0.061 mmol), potassium ((3-((tert-butyldimethylsilyl)oxy)azetidin-1-yl)methyl)trifluoroborate (37.8 mg, 0.123 mmol, Pd(OAc)₂ (2.069 mg, 9.21 μmol), Xphos (8.79 mg, 0.018 mmol) and Cs₂CO₃ (60.0 mg, 0.184 mmol) were added to a reaction tube. Under N₂, THF (1.5 mL) and water (0.15 mL) were added and the mixture stirred at 90° C. for 16 hrs. The solvent was evaporate to give crude 5-((3-((tert-butyldimethylsilyl)oxy)azetidin-1-yl)methyl)-2-(1-(5-fluoro-4-methoxypyridin-2-yl)ethyl)-7-((2-(methylamino)-1H-imidazol-1-yl)methyl)-3,4-dihydroisoquinolin-1(2H)-one (51-1). LC-MS: MS m/z 609.4 [M+H]⁺.
Note: potassium ((3-((tert-butyldimethylsilyl)oxy)azetidin-1-yl)methyl)trifluoroborate was obtained from 3-((tert-butyldimethylsilyl)oxy)azetidine using the procedure described in step 1 of Example 7).

Step 2: To a solution of compound 51-1 (37 mg, 0.061 mmol) in THF (1 mL) was added TBAF (0.182 mL, 0.182 mmol). The mixture was stirred at rt for 4 hrs and then purified by prep-HPLC to give a racemic mixture (13.2 mg, 37.8%) which was then separated by chiral SFC (Column: (R,R)-WHELKO1, 5 um, 4.6 mm*250 mm, Condition: EtOH+0.5% NH₄OH) to give (S)-2-(1-(5-fluoro-4-methoxypyridin-2-yl)ethyl)-5-((3-hydroxyazetidin-1-yl)methyl)-7-((2-(methylamino)-1H-imidazol-1-yl)methyl)-3,4-dihydroisoquinolin-1(2H)-one (51) (the 2nd peak on the prep SFC). ¹H NMR (400 MHz, Methanol-d₄) δ ppm 8.24 (d, J=3.3 Hz, 1H), 7.80 (d, J=1.7 Hz, 1H), 7.19 (d, J=1.8 Hz, 1H), 7.16 (d, J=6.9 Hz, 1H), 6.63 (d, J=1.7 Hz, 1H), 6.59 (d, J=1.7 Hz, 1H), 5.98 (q, J=7.0 Hz, 1H), 4.96 (s, 2H), 4.31 (t, J=6.3 Hz, 1H), 3.94 (s, 3H), 3.63 (s, 2H), 3.56 (dd, J=9.4, 4.6 Hz, 3H), 3.44-3.35 (m, 1H), 2.97-2.88 (m, 3H), 2.85 (s, 4H), 1.62 (d, J=7.1 Hz, 3H). LC-MS: [M+H]⁺=495.3.

### Example 52: Synthesis of 5-(2-chloro-5-(hydroxymethyl)pyridin-3-yl)-2-(3-methoxybenzyl)-7-((2-(methylamino)-1H-imidazol-1-yl)methyl)-3,4-dihydroisoquinolin-1(2H)-one (52)

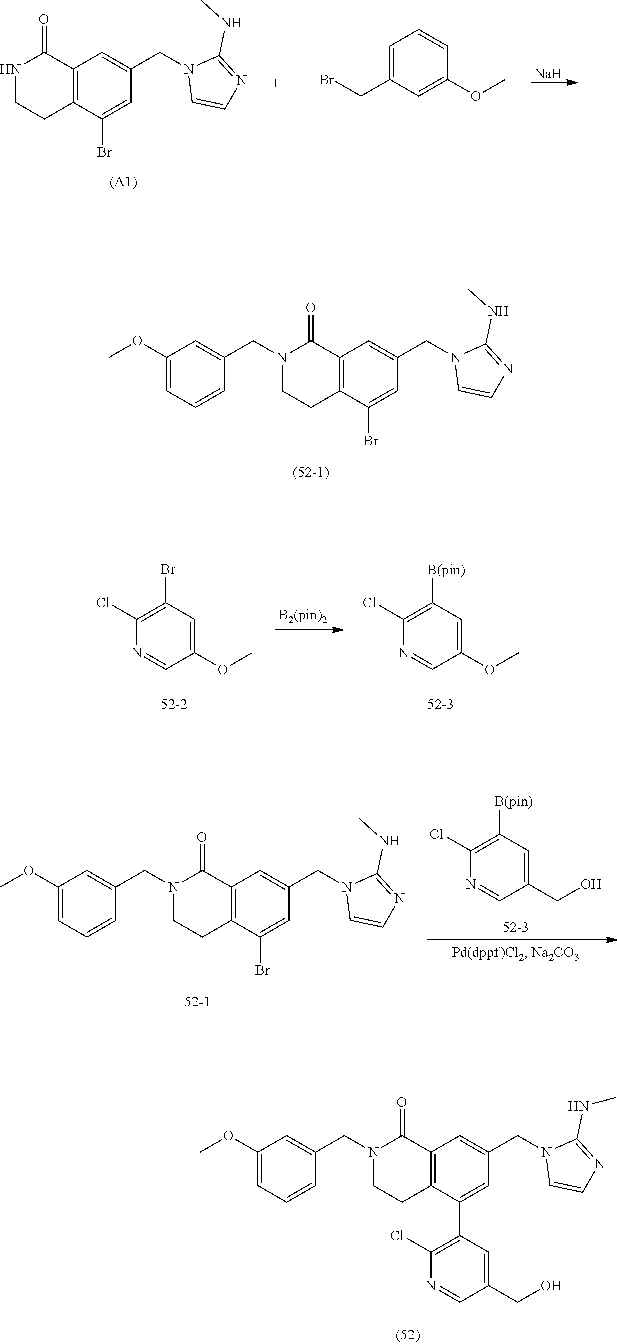

Step 1: Synthesis of 5-bromo-2-(3-methoxybenzyl)-7-((2-(methylamino)-1H-imidazol-1-yl)methyl)-3,4-dihydroisoquinolin-1(2H)-one (52-1).

5-bromo-2-(3-methoxybenzyl)-7-((2-(methylamino)-1H-imidazol-1-yl)methyl)-3,4-dihydroisoquinolin-1(2H)-one (52-1) was obtained using the procedure described in Example 1 using intermediate (A1) but replacing intermediate (G1) with 1-(bromomethyl)-3-methoxybenzene. LC-MS: [M+H]⁺=454.9.

Step 2: Synthesis of compound 52-3

Potassium acetate (476 mg, 4.85 mmol) was added to the solution of (5-bromo-6-chloropyridin-3-yl)methanol (52-2) (540 mg, 2.427 mmol) and 4,4,4′,4′,5,5,5′,5′-octamethyl-2,2′-bi(1,3,2-dioxaborolane) (1541 mg, 6.07 mmol) in dioxane (15 mL). The mixture was degassed for 10 min, followed by the addition of PdCl₂(dppf).CH₂Cl₂ adduct (139 mg, 0.170 mmol). The mixture was heated and stirred at 80° C. for 17 hr under nitrogen atmosphere. The solution was filtered, the filtrate was concentrated to give crude compound 52-3. LC-MS: [M+H]⁺=270.1.

Step 3: Synthesis of 5-(2-chloro-5-(hydroxymethyl)pyridin-3-yl)-2-(3-methoxybenzyl)-7-((2-(methylamino)-1H-imidazol-1-yl)methyl)-3,4-dihydroisoquinolin-1(2H)-one (52) using Suzuki coupling.

To a mixture of compound 52-1 (75 mg, 0.16 mmol) in dioxane/H₂O (10/1, 5.5 mL) were added compound 52-3 (62 mg, 0.33 mmol, 2.0 eq), Na₂CO₃ (35 mg, 0.33 mmol, 2.0 eq) and Pd(dppf)Cl₂ (24 mg, 0.03 mmol, 0.2 eq) under nitrogen atmosphere. The mixture was stirred at 90° C. for 2 hrs under nitrogen atmosphere. The solution was filtered and the filtrate was concentrated, the crude was purified by preparative HPLC (Column: Boston Green ODS 150*30 mm*5 um, gradient: 26-36% B (A=0.1% TFA water, B=acetonitrile), flow rate: 30 mL/min) to afford 5-(2-chloro-5-(hydroxymethyl)pyridin-3-yl)-2-(3-methoxybenzyl)-7-((2-(methylamino)-1H-imidazol-1-yl)methyl)-3,4-dihydroisoquinolin-1(2H)-one (52) (66.1 mg, 78% yield) as a brown solid. ¹H NMR (400 MHz, Methanol-d₄) δ 8.44 (d, J=2.0 Hz, 1H), 7.98 (d, J=2.0 Hz, 1H), 7.77 (d, J=2.0 Hz, 1H), 7.36 (d, J=2.0 Hz, 1H), 7.28-7.24 (m, 1H), 7.01 (t, J=2.4 Hz, 2H), 7.00-6.88 (m, 3H), 5.20 (s, 2H), 4.84-4.74 (m, 2H), 4.71 (d, J=3.6 Hz, 2H), 3.79 (s, 3H), 3.53-3.49 (m, 2H), 3.03 (s, 3H), 2.75-2.71 (s, 2H). LCMS: [M+H]⁺=518.0.
Following a similar procedure to that of Example 52 (unless otherwise specified). The following compounds were prepared from their corresponding precursors.

| Ex. | Name/Structure | ¹HNMR/LC-MS | Precursors |
|---|---|---|---|
| 53 | 5-(2-chloro-5- | 1H NMR (400 MHz, Methanol-d4) | Step 1: |
| | (hydroxymethyl)pyridin-3-yl)-2-(4- | δ ppm 8.44 (d, J = 2.0 Hz, 1H), | intermediate (A1) |
| | fluoro-3-methoxybenzyl)-7-((2- | 7.98 (d, J = 1.6 Hz, 1H), 7.77 (d, | and 4- |
| | (methylamino)-1H-imidazol-1- | J = 2.4 Hz, 1H), 7.37 (s, 1H), 7.15- | (bromomethyl)-1- |
| | yl)methyl)-3,4-dihydroisoquinolin- | 7.05 (m, 2H), 7.04-6.95 (m, 2H), | fluoro-2- |
| | 1(2H)-one | 6.95-6.88 (m, 1H), 5.20 (s, 2H), | methoxybenzene |
| | 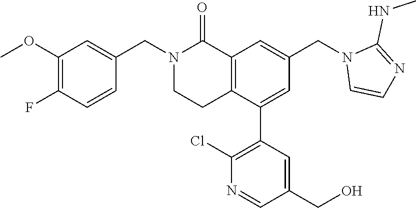 | 4.84-4.74 (m, 4H), 3.86 (s, 3H), 3.55-3.45 (m, 2H), 3.03 (s, 3H), 2.80-2.71 (s, 2H). LC-MS: (ESI) m/z 536.1 [M + H]+ | Step 2: (5-bromo-6- chloropyridin-3- yl)methanol (52-2) |
| 54 | 2-(4-fluoro-3-methoxybenzyl)-5-(1- | ¹H NMR (400 MHz, Methanol-d₄) | Step 1: |
| | methyl-3-(trifluoromethyl)-1H- | δ ppm 8.53 (s, 1H), 7.92 (d, J = | intermediate (A1) |
| | pyrazol-4-yl)-7-((2-(methylamino)- | 1.9 Hz, 1H), 7.75 (d, J = 1.0 Hz, | and 4- |
| | 1H-imidazol-1-yl)methyl)-3,4- | 1H), 7.20 (d, J = 1.9 Hz, 1H), 7.10- | (bromomethyl)-1- |
| | dihydroisoquinolin-1(2H)-one | 7.00 (m, 2H), 6.88 (ddd, J = 8.3, | fluoro-2- |
| | 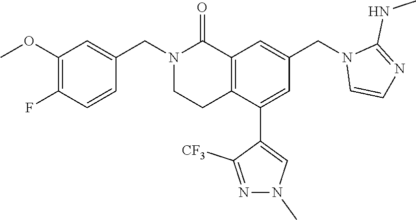 | 4.2, 2.1 Hz, 1H), 6.80 (s, 2H), 5.08 (s, 2H), 4.73 (s, 2H), 3.97 (s, 3H), 3.84 (s, 3H), 3.47 (t, J = 6.6 Hz, 2H), 2.91 (s, 3H), 2.78 (t, J = 6.6 Hz, 2H). LC-MS: (ESI) m/z 543.3 [M + H]⁺ | methoxybenzene Step 2: Compound 52-2 was replaced with (4-bromo-1- methyl-3- (trifluoromethyl)- 1H-pyrazole |
| 55 | 2-(4-fluoro-3-methoxybenzyl)-5-(3- | ¹H NMR (400 MHz, Methanol-d₄) | Step 1: |
| | methyl-1H-pyrazol-4-yl)-7-((2- | δ ppm 8.51 (s, 2H), 7.85 (d, J = | intermediate (A1) |
| | (methylamino)-1H-imidazol-1- | 2.0 Hz, 1H), 7.52 (s, 1H), 7.27 (d, | and 4- |
| | yl)methyl)-3,4-dihydroisoquinolin- | J = 2.2 Hz, 1H), 7.11-7.00 (m, | (bromomethyl)-1- |
| | 1(2H)-one | 2H), 6.89 (dt, J = 8.2, 2.4 Hz, 3H), | fluoro-2- |
| | 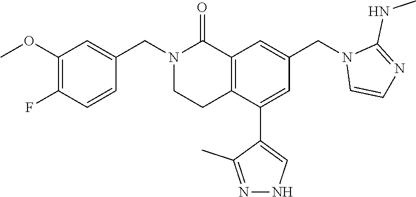 | 5.11 (s, 2H), 4.74 (s, 2H), 3.84 (s, 3H), 3.48 (t, J = 6.6 Hz, 2H), 2.96 (s, 3H), 2.87 (t, J = 6.6 Hz, 2H), 2.17 (s, 3H). LC-MS: (ESI) m/z 474.8 [M + H]⁺ | methoxybenzene Step 3: Compound 52-3 was replaced with (3-methyl-1H- pyrazol-4- yl)boronic acid |
| 56 | (S)-5-(2-chloro-5- | ¹H NMR (400 MHz, Methanol-d₄) | Step 1: |
| | (hydroxymethyl)pyridin-3-yl)-2-(1-(4- | δ ppm 8.44 (d, J = 2.0 Hz, 1H), | intermediate (A1) |
| | fluoro-3-methoxyphenyl)ethyl)-7-((2- | 7.98 (d, J = 1.6 Hz, 1H), 7.77 (d, | and 4-(1- |
| | (methylamino)-1H-imidazol-1- | J = 2.4 Hz, 1H), 7.37 (s, 1H), | bromoethyl)-1- |
| | yl)methyl)-3,4-dihydroisoquinolin- | 7.15-7.05 (m, 2H), 7.04-6.95 (m, | fluoro-2- |
| | 1(2H)-one | 2H), 6.95-6.88 (m, 1H), 5.96 (q, | methoxybenzene |
| | 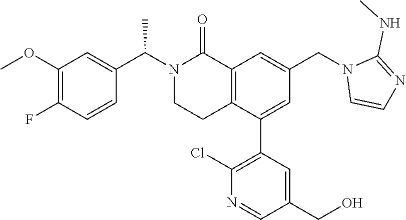 [SMILES: CNc1nccn1Cc1cc2c(c(-c3cc(CO)cnc3Cl)c1)CCN([C@@H](C)c1ccc(F)c(OC)c1)C2=O] | J = 7.1 Hz, 1H), 4.84-4.74 (m, 4H), 3.86 (s, 3H), 3.55-3.45 (m, 2H), 3.03 (s, 3H), 2.80-2.71 (s, 2H), 1.61 (d, J = 7.1 Hz, 3H). LC-MS: (ESI) m/z 550.2 [M + H]⁺ | Step 2: 5-bromo-6- chloropyridin-3- yl)methanol (52-2) (followed by SFC separation) |
| 57 | (R)-5-(2-chloro-5- | ¹H NMR (400 MHz, Methanol-d₄) | Step 1: |
| | (hydroxymethyl)pyridin-3-yl)-2-(1-(4- | δ ppm 8.42 (s, 1H), 7.97 (s, 1H), | intermediate (A1) |
| | fluoro-3-methoxyphenyl)ethyl)-7-((2- | 7.77-7.72 (m, 1H), 7.35 (s, 1H), | and 4-(1- |
| | (methylamino)-1H-imidazol-1- | 7.07-7.06 (m, 2H), 7.02-6.96 (m, | bromoethyl)-1- |
| | yl)methyl)-3,4-dihydroisoquinolin- | 2H), 6.08-6.05 (m, 1H), 5.19 (s, | fluoro-2- |
| | 1(2H)-one | 2H), 4.70 (d, J = 9.2 Hz, 2H), 3.85 | methoxybenzene |
| | 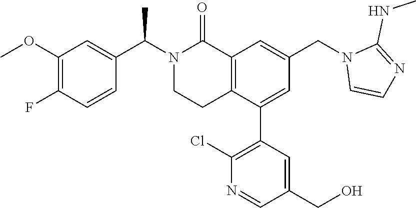 | (s, 3H), 3.33-3.31 (m, 1H), 3.14- 3.13 (m, 1H), 3.02 (s, 3H), 2.65- 2.62 (m, 2H), 1.62-1.59 (m, 3H). LC-MS: (ESI) m/z 550.2 [M + H]⁺ | Step 2: 5-bromo-6- chloropyridin-3- yl)methanol (52-2) (followed by SFC separation) |
| 58 | 5-(1,3-dimethyl-1H-pyrazol-4-yl)-2- | ¹H NMR (400 MHz, Methanol-d₄) | Step 1: |
| | (4-fluoro-3-methoxybenzyl)-7-((2- | δ ppm 8.52 (s, 1H), 7.85 (d, J = | intermediate (A1) |
| | (methylamino)-1H-imidazol-1- | 2.0 Hz, 1H), 7.54 (s, 1H), 7.22 (d, | and 4- |
| | yl)methyl)-3,4-dihydroisoquinolin- | J = 2.0 Hz, 1H), 7.06 (qd, J = 8.3, | (bromomethyl)-1- |
| | 1(2H)-one | 3.9 Hz, 2H), 6.94-6.77 (m, 3H), | fluoro-2- |
| | 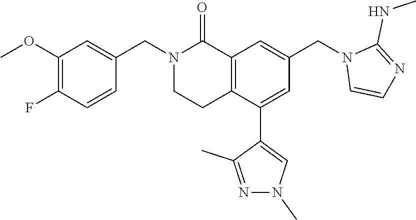 | 5.08 (d, J = 5.0 Hz, 2H), 4.73 (s, 2H), 3.84 (d, J = 4.9 Hz, 6H), 3.47 (t, J = 6.6 Hz, 2H), 2.93 (s, 3H), 2.86 (t, J = 6.6 Hz, 2H), 2.09 (s, 3H). LC-MS: (ESI) m/z 489.2 [M + H]⁺ | methoxybenzene Step 3: Compound 52-3 was replaced with (1,3-dimethyl-1H- pyrazol-4- yl)boronic acid |
| 59 | (S)-2-(1-(5-fluoro-4-methoxypyridin- | 1H NMR (400 MHz, Methanol-d4) | Step 1: |
| | 2-yl)ethyl)-7-((2-(methylamino)-1H- | δ 9.19 (s, 1H), 8.83 (s, 2H), 8.23 | intermediate (A1) |
| | imidazol-1-yl)methyl)-5-(pyrimidin-5- | (d, J = 3.2 Hz, 1H), 7.95 (s, 1H), | and intermediate |
| | yl)-3,4-dihydroisoquinolin-1(2H)-one | 7.46 (s, 1H), 7.18 (d, J = 6.9 Hz, | (G9) |
| | 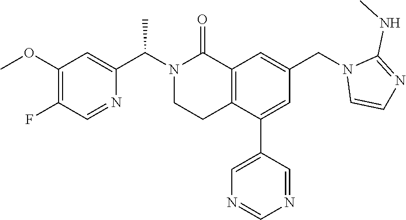 [SMILES: CNc1nccn1Cc1cc2c(c(-c3cncnc3)c1)CCN([C@@H](C)c1cc(OC)c(F)cn1)C2=O] | 1H), 6.89 (d, J = 10.4 Hz, 2H), 5.98 (q, J = 7.0 Hz, 1H), 5.14 (s, 2H), 3.96 (s, 3H), 3.49 (d, J = 6.5 Hz, 1H), 3.39 (d, J = 6.3 Hz, 1H), 2.96 (s, 3H), 2.89 (t, J = 6.5 Hz, 2H), 1.63 (d, J = 7.1 Hz, 3H). LC-MS: (ESI) m/z 488.3 [M + H]⁺ | Step 3: Compound 52-3 was replaced with pyrimidin-5- ylboronic acid (followed by SFC separation) |
| 60 | 2-((5-fluoro-4-methoxypyridin-2- | ¹H NMR (400 MHz, Methanol-d4) | Step 1: |
| | yl)methyl)-5-(1-methyl-1H-pyrazol-5- | δ ppm 8.29 (d, J = 3.6 Hz, 1H), | intermediate (A1) |
| | yl)-7-((2-(methylamino)-1H-imidazol- | 7.94 (s, 1H), 7.56 (d, J = 1.9 Hz, | and intermediate |
| | 1-yl)methyl)-3,4-dihydroisoquinolin- | 1H), 7.44 (d, J = 1.7 Hz, 1H), 7.27 | (G10) |
| | 1(2H)-one | (d, J = 7.0 Hz, 1H), 6.99 (s, 2H), | Step 3: |
| | 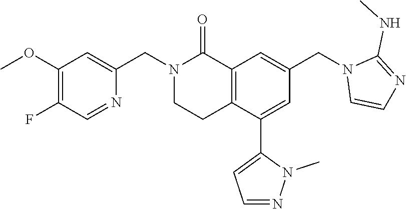 [SMILES: CNc1nccn1Cc1cc2c(c(-c3ccnn3C)c1)CCN(Cc1cc(OC)c(F)cn1)C2=O] | 6.34 (d, J = 1.9 Hz, 1H), 5.17(s, 2H), 4.85 (s, 2H), 4.00 (s, 3H), 3.66 (d, J = 9.3 Hz, 5H), 3.00 (s, 3H), 2.86 (t, J = 6.5 Hz, 2H). LC-MS: (ESI) m/z 476.2 [M + H]⁺ | Compound 52-3 was replaced with (1-methyl-1H- pyrazol-5- yl)boronic acid |
| 61 | 5-(2-chloro-5- | ¹H NMR (400 MHz, Methanol-d₄) | Step 1: |
| | (hydroxymethyl)pyridin-3-yl)-2-(2,4- | δ ppm 8.44 (d, J = 2.0 Hz, 1H), | intermediate (A1) |
| | difluoro-5-methoxybenzyl)-7-((2- | 7.96 (s, 1H), 7.77 (d, J = 2.0 Hz, | and 1- |
| | (methylamino)-1H-imidazol-1- | 1H), 7.36 (d, J = 1.6 Hz, 1H), | (bromomethyl)- |
| | yl)methyl)-3,4-dihydroisoquinolin- | 7.17-7.14 (m, 1H), 7.05-6.99 (m, | 2,4-difluoro-5- |
| | 1(2H)-one | 3H), 5.19 (s, 2H), 4.95-4.75 (m, | methoxybenzene |
| | 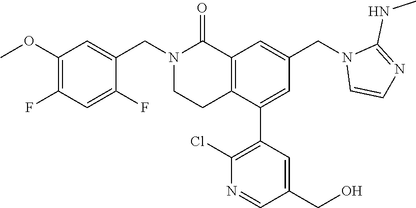 | 2H), 4.71 (s, 2H), 3.86 (s, 3H), 3.59-3.55 (m, 2H), 3.02 (s, 3H), 2.79-2.69 (m, 2H). LC-MS: (ESI) m/z 554.2 [M + H]⁺ | Step 2: Compound 52-2 was replaced with (5-bromo-6- chloropyridin-3- yl)methanol |
| 62 | (S)-5-(1,3-dimethyl-1H-pyrazol-4-yl)- | 1H NMR (400 MHz, Methanol-d4) | Step 1: |
| | 2-(1-(5-fluoro-4-methoxypyridin-2- | δ 8.23 (d, J = 3.3 Hz, 1H), 7.84 | intermediate (A1) |
| | yl)ethyl)-7-((2-(methylamino)-1H- | (d, J = 1.9 Hz, 1H), 7.51 (s, 1H), | and intermediate |
| | imidazol-1-yl)methyl)-3,4- | 7.20-7.11 (m, 2H), 6.73 (d, J = | (G9) |
| | dihydroisoquinolin-1(2H)-one | 1.9 Hz, 1H), 6.69 (d, J = 1.9 Hz, | Step 3: |
| | 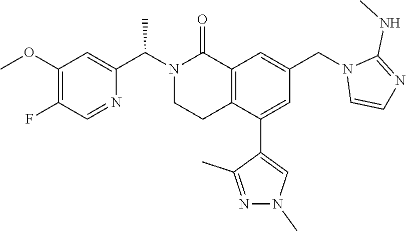 [SMILES: CNc1nccn1Cc1cc2c(c(-c3cn(C)nc3C)c1)CCN([C@@H](C)c1cc(OC)c(F)cn1)C2=O] | 1H), 5.98 (q, J = 7.1 Hz, 1H), 5.03 (s, 2H), 3.95 (s, 3H), 3.84 (s, 3H), 3.47 (dd, J = 8.4, 4.7 Hz, 1H), 3.38-3.32 (m, 1H), 2.88 (s, 3H), 2.83-2.71 (m, 2H), 2.08 (s, 3H), 1.62 (d, J = 7.1 Hz, 3H). LC-MS: (ESI) m/z 504.3 [M + H]⁺ | Compound 52-3 was replaced with (1,3-dimethyl-1H- pyrazol-4- yl)boronic acid (followed bySFC separation) |
| 63 | (S)-2-(1-(5-fluoro-4-methoxypyridin- | ¹H NMR (400 MHz, Methanol-d4) | Step 1: |
| | 2-yl)ethyl)-7-((2-(methylamino)-1H- | δ 8.23 (d, J = 3.3 Hz, 1H), 7.87 (s, | intermediate (A1) |
| | imidazol-1-yl)methyl)-5-(1H-pyrazol- | 1H), 7.72 (s, 1H), 7.50 (s, 1H), | and intermediate |
| | 5-yl)-3,4-dihydroisoquinolin-1(2H)- | 7.17 (d, J = 6.9 Hz, 1H), 6.69 (d, | (G9) |
| | one | J = 1.8 Hz, 1H), 6.63 (d, J = 1.8 Hz, | Step 3: |
| | 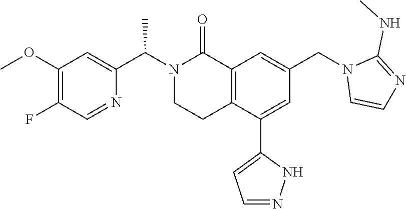 [SMILES: CNc1nccn1Cc1cc2c(c(-c3ccn[nH]3)c1)CCN([C@@H](C)c1cc(OC)c(F)cn1)C2=O] | 1H), 6.44 (s, 1H), 5.99 (q, J = 7.1 Hz, 1H), 5.03 (s, 2H), 3.95 (s, 3H), 3.58-3.45 (m, 1H), 3.36 (dd, J = 11.6, 4.7 Hz, 1H), 3.14-2.93 (m, 2H), 2.87 (s, 3H), 1.62 (d, J = 7.1 Hz, 3H). LC-MS: (ESI) m/z 476.0 [M + H]⁺ | Compound 52-3 was replaced with (1H-pyrazol-5- yl)boronic acid (followed by SFC separation) |
| 64 | 2-((5-fluoro-4-methoxypyridin-2- | ¹HNMR (400 MHz, Methanol-d₄) δ | Step 1: |
| | yl)methyl)-5-(1-methyl-3- | ppm 8.50 (d, J = 4.4 Hz, 1H), 7.92 | intermediate (A1) |
| | (trifluoromethyl)-1H-pyrazol-4-yl)-7- | (d, J = 2.0 Hz, 1H), 7.79 (s, 1H), | and intermediate |
| | ((2-(methylamino)-1H-imidazol-1- | 7.47 (d, J = 7.2 Hz, 1H), 7.30 (d, | (G10) |
| | yl)methyl)-3,4-dihydroisoquinolin- | J = 1.2 Hz, 1H), 7.03-6.91 (m, 2H), | Step 2: |
| | 1(2H)-one | 5.16 (s, 2H), 4.92 (s, 2H), 4.10 (s, | Compound 52-2 |
| | 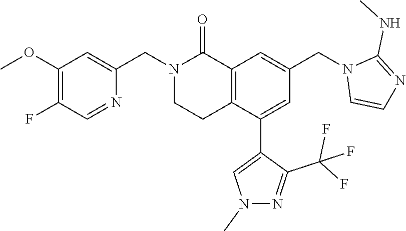 [SMILES: CNc1nccn1Cc1cc2c(c(-c3cn(C)nc3C(F)(F)F)c1)CCN(Cc1cc(OC)c(F)cn1)C2=O] | 3H), 4.00 (s, 3H), 3.68 (t, J = 6.8 Hz, 2H), 2.99 (s, 3H), 2.92 (t, J = 6.8 Hz, 2H). LC-MS: (ESI) m/z 544.0 [M + H]⁺ | was replaced with 4-bromo-1-methyl- 3-(trifluoromethyl)- 1H-pyrazole |
| 65 | 5-(2-chloro-5- | ¹H NMR (400 MHz, Methanol-d₄) | Step 1: |
| | (hydroxymethyl)phenyl)-2-(4-fluoro- | δ ppm 7.91 (d, J = 1.7 Hz, 1H), | intermediate (A1) |
| | 3-methoxybenzyl)-7-((2- | 7.49 (d, J = 8.3 Hz, 1H), 7.39 (dd, | and 4- |
| | (methylamino)-1H-imidazol-1- | J = 8.3, 2.0 Hz, 1H), 7.28 (dd, J = | (bromomethyl)-1- |
| | yl)methyl)-3,4-dihydroisoquinolin- | 11.0, 1.8 Hz, 2H), 7.11-7.01 (m, | fluoro-2- |
| | 1(2H)-one | 2H), 6.99 (d, J = 1.0 Hz, 2H), 6.91- | methoxybenzene |
| | 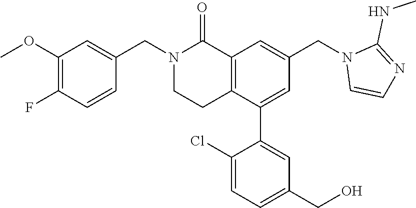 | 6.79 (m, 1H), 5.16 (s, 2H), 4.80- 4.66 (m, 2H), 4.61 (s, 2H), 3.84 (s, 3H), 3.48 (t, J = 6.7 Hz, 2H), 3.00 (s, 3H), 2.77-2.59 (m, 2H). LC-MS: (ESI) m/z 534.8 [M + H]⁺ | Step 2: Compound 52-2 was replaced with (3-bromo-4- chlorophenyl) methanol |
| 66 | 5-(3-(difluoromethyl)-1-methyl-1H- | ¹H NMR (400 MHz, Methanol-d₄) | Step 1: |
| | pyrazol-4-yl)-2-(4-fluoro-3- | δ ppm 7.91 (s, 1H), 7.72 (s, 1H), | intermediate (A1) |
| | methoxybenzyl)-7-((2- | 7.30 (s, 1H), 7.17-6.87 (m, 4H), | and 4- |
| | (methylamino)-1H-imidazol-1- | 6.66 (t, J = 55.3 Hz, 1H), 5.16 (s, | (bromomethyl)-1- |
| | yl)methyl)-3,4-dihydroisoquinolin- | 2H), 4.74 (s, 2H), 3.95 (s, 3H), | fluoro-2- |
| | 1(2H)-one | 3.84 (s, 3H), 3.48 (m, 2H), 2.99 | methoxybenzene |
| | 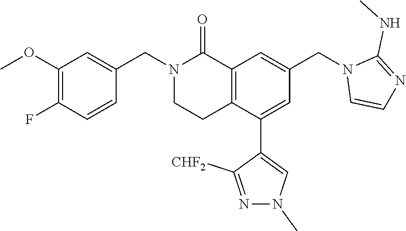 [SMILES: CNc1nccn1Cc1cc2c(c(-c3cn(C)nc3C(F)F)c1)CCN(Cc1ccc(F)c(OC)c1)C2=O] | (s, 3H), 2.86 (m, 2H). LC-MS: (ESI) m/z 525.4 [M + H]⁺ | Step 2: Compound 52-2 was replaced with 4-bromo-3- (difluoromethyl)-1- methyl-1H- pyrazole |
| 67 | 5-(2-chloro-5- | ¹H NMR (400 MHz, Methanol-d₄) | Step 1: |
| | (hydroxymethyl)pyridin-3-yl)-2-(3- | δ ppm 8.42 (d, J = 2.3 Hz, 1H), | intermediate (A1) |
| | chloro-5-fluorobenzyl)-7-((2- | 7.96 (d, J = 1.7 Hz, 1H), 7.77 (d, | and 1- |
| | (methylamino)-1H-imidazol-1- | J = 2.3 Hz, 1H), 7.36 (d, J = 1.8 Hz, | (bromomethyl)-3- |
| | yl)methyl)-3,4-dihydroisoquinolin- | 1H), 7.21 (s, 1H), 7.14 (dt, J = | chloro-5- |
| | 1(2H)-one | 8.6, 2.0 Hz, 1H), 7.07 (d, J = 9.2 | fluorobenzene |
| | 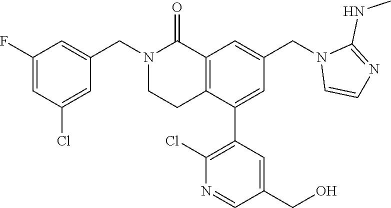 [SMILES: CNc1nccn1Cc1cc2c(c(-c3cc(CO)cnc3Cl)c1)CCN(Cc1cc(F)cc(Cl)c1)C2=O] | Hz, 1H), 6.99 (d, J = 2.7 Hz, 2H), 5.18 (s, 2H), 4.83 (d, J = 15.1 Hz, 1H), 4.71 (d, J = 13.8 Hz, 3H), 3.59-3.50 (m, 2H), 3.00 (s, 3H), 2.76 (dd, J = 12.1, 6.3 Hz, 2H). LC-MS: (ESI) m/z 539.8 [M + H]⁺ | Step 2: Compound 52-2 |
| 68 | 5-(2-chloro-5- | ¹H NMR (400 MHz, Methanol-d₄) | Step 1: |
| | (hydroxymethyl)pyridin-3-yl)-2-((5- | δ ppm 8.40 (d, J = 2.3 Hz, 1H), | intermediate (A1) |
| | fluoro-4-methoxypyridin-2-yl)methyl)- | 8.20 (d, J = 3.4 Hz, 1H), 7.95 (d, | and 1 intermediate |
| | 7-((2-(methylamino)-1H-imidazol-1- | J = 1.7 Hz, 1H), 7.73 (d, J = 2.3 Hz, | (G10) |
| | yl)methyl)-3,4-dihydroisoquinolin- | 1H), 7.18 (d, J = 6.9 Hz, 2H), 6.66 | Step 2: |
| | 1(2H)-one | (d, J = 1.7 Hz, 1H), 6.58 (d, J = | Compound 52-2 |
| | 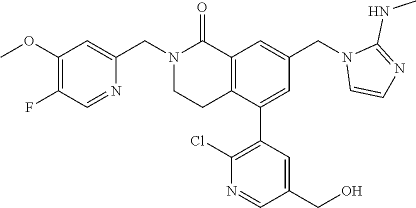 | 1.7 Hz, 1H), 5.03 (s, 2H), 4.81 (d, J = 6.0 Hz, 2H), 4.68 (s, 2H), 3.95 (s, 3H), 3.65-3.56 (m, 2H), 2.84 (s, 3H), 2.74 (dt, J = 20.4, 7.1 Hz, 2H). LC-MS: (ESI) m/z 537.2 [M + H]⁺ | |
| 69 | 5-((5-(2-chloro-5- | ¹H NMR (400 MHz, Methanol-d₄) | Step 1: |
| | (hydroxymethyl)pyridin-3-yl)-7-((2- | δ ppm 8.51 (s, 1H), 8.41 (d, J = | intermediate (A1) |
| | (methylamino)-1H-imidazol-1- | 2.3 Hz, 1H), 7.95 (d, J = 1.6 Hz, | and 5- |
| | yl)methyl)-1-oxo-3,4- | 1H), 7.79-7.62 (m, 3H), 7.40- | (bromomethyl)-2- |
| | dihydroisoquinolin-2(1H)-yl)methyl)- | 7.24 (m, 2H), 6.86 (d, J = 5.8 Hz, | fluorobenzonitrile |
| | 2-fluorobenzonitrile | 2H), 5.12 (s, 2H), 4.83 (d, J = | Step 2: |
| | 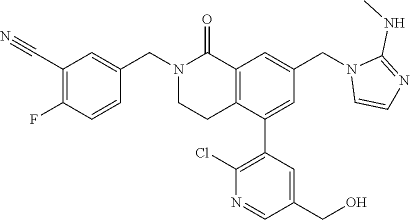 [SMILES: CNc1nccn1Cc1cc2c(c(-c3cc(CO)cnc3Cl)c1)CCN(Cc1ccc(F)c(C#N)c1)C2=O] | 15.0 Hz, 1H), 4.74 (d, J = 15.0 Hz, 1H), 4.68 (s, 2H), 3.61-3.50 (m, 2H), 2.95 (s, 3H), 2.75 (dd, J = 12.3, 6.2 Hz, 2H). LC-MS: (ESI) m/z 531.2 [M + H]⁺ | Compound 52-2 |
| 70 | 5-(2-chloro-5- | ¹H NMR (500 MHz, Methanol-d4) | Step 1: |
| | (hydroxymethyl)pyridin-3-yl)-2-(3,4- | δ ppm 8.51 (s, 1H), 8.41 (d, J = | intermediate (A1) |
| | difluorobenzyl)-7-((2-(methylamino)- | 2.3 Hz, 1H), 7.96 (d, J = 1.7 Hz, | and 4- |
| | 1H-imidazol-1-yl)methyl)-3,4- | 1H), 7.74 (d, J = 2.3 Hz, 1H), 7.33- | (bromomethyl)- |
| | dihydroisoquinolin-1(2H)-one | 7.19 (m, 3H), 7.16 (s, 1H), 6.93- | 1,2- |
| | 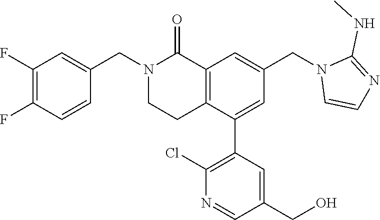 [SMILES: CNc1nccn1Cc1cc2c(c(-c3cc(CO)cnc3Cl)c1)CCN(Cc1ccc(F)c(F)c1)C2=O] | 6.81 (m, 2H), 5.13 (s, 2H), 4.79 (d J = 14.9 Hz 1H) 4.71 (d, J = 2.5 Hz, 1H), 4.68 (s, 2H), 3.53- 3.47 (m, 2H), 2.96 (s, 3H), 2.78- 2.66 (m, 2H). LC-MS: (ESI) m/z 523.7 [M + H]⁺ | difluorobenzene Step 2: Compound 52-2 |
| 71 | (R)-5-(1,3-dimethyl-1H-pyrazol-4-yl)- | 1H NMR (400 MHz, Methanol-d4) | Step 1: |
| | 2-(1-(5-fluoro-4-methoxypyridin-2- | δ 8.23 (d, J = 3.3 Hz, 1H), 7.84 | intermediate (A1) |
| | yl)ethyl)-7-((2-(methylamino)-1H- | (d, J = 1.9 Hz, 1H), 7.51 (s, 1H), | and intermediate |
| | imidazol-1-yl)methyl)-3,4- | 7.20-7.11 (m, 2H), 6.73 (d, J = | (G9) |
| | dihydroisoquinolin-1(2H)-one | 1.9 Hz, 1H), 6.69 (d, J = 1.9 Hz, | Step 3: |
| | 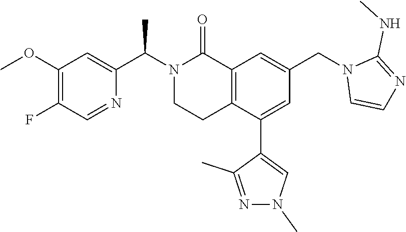 [SMILES: CNc1nccn1Cc1cc2c(c(-c3cn(C)nc3C)c1)CCN([C@H](C)c1cc(OC)c(F)cn1)C2=O] | 1H), 5.98 (q, J = 7.1 Hz, 1H), 5.03 (s, 2H), 3.95 (s, 3H), 3.84 (s, 3H), 3.47 (dd, J = 8.4, 4.7 Hz, 1H), 3.38-3.32 (m, 1H), 2.88 (s, 3H), 2.83-2.71 (m, 2H), 2.08 (s, 3H), 1.62 (d, J = 7.1 Hz, 3H). LC-MS: (ESI) m/z 504.3 [M + H]⁺ | Compound 52-3 was replaced with (1,3-dimethyl-1H- pyrazol-4- yl)boronic acid (followed by SFC separation) |
| 72 | 2-(4-fluoro-3-methoxybenzyl)-5-(1- | ¹H NMR (400 MHz, Methanol-d₄) | Step 1: |
| | methyl-1H-pyrazol-5-yl)-7-((2- | δ ppm 7.98 (s, 1H), 7.54 (d, J = | intermediate (A1) |
| | (methylamino)-1H-imidazol-1- | 1.9 Hz, 1H), 7.37 (s, 1H), 7.15- | and 4- |
| | yl)methyl)-3,4-dihydroisoquinolin- | 6.97 (m, 2H), 6.92 (dd, J = 9.0, | (bromomethyl)-1- |
| | 1(2H)-one | 2.2 Hz, 3H), 6.32 (d, J = 1.9 Hz, | fluoro-2- |
| | 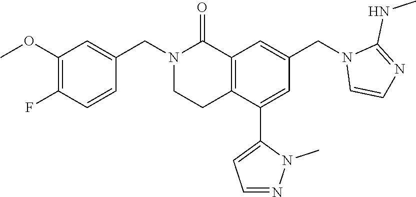 | 1H), 5.14 (s, 2H), 4.74 (s, 2H), 3.84 (s, 3H), 3.64 (s, 3H), 3.50 (t, J = 6.6 Hz, 2H), 2.97 (s, 3H), 2.78 (t, J = 6.5 Hz, 2H). LC-MS: (ESI) m/z 474.8 [M + H]⁺ | methoxybenzene Step 3: Compound 52-3 was replaced with (1-methyl-1H- pyrazol-5- yl)boronic acid |
| 73 | 5-(2-chloro-5- | ¹H NMR (400 MHz, Methanol-d₄) | Step 1: |
| | (hydroxymethyl)pyridin-3-yl)-2-(3- | δ ppm 8.44 (d, J = 2.0 Hz, 1H), | intermediate (A1) |
| | (difluoromethoxy)benzyl)-7-((2- | 7.98 (d, J = 1.6 Hz, 1H), 7.78 (d, | and 1- |
| | (methylamino)-1H-imidazol-1- | J = 2.0 Hz, 1H), 7.40-7.37 (m, 2H), | (bromomethyl)-3- |
| | yl)methyl)-3,4-dihydroisoquinolin- | 7.22 (d, J = 7.6 Hz, 1H), 7.14- | (difluoromethoxy) |
| | 1(2H)-one | 7.10 (m, 2H), 7.02-6.65 (m, 3H), | benzene |
| | 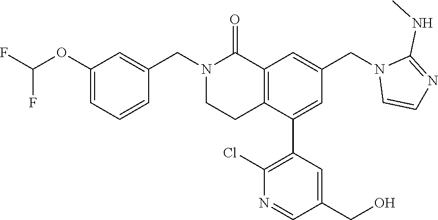 [SMILES: CNc1nccn1Cc1cc2c(c(-c3cc(CO)cnc3Cl)c1)CCN(Cc1cccc(OC(F)F)c1)C2=O] | 5.20 (s, 2H), 4.85-4.74 (m, 2H), 4.71 (s, 2H), 3.57-3.50 (m, 2H), 3.03 (s, 3H), 2.85-2.72 (m, 2H). LC-MS: (ESI) m/z 554.1 [M + H]⁺ | Step 2: Compound 52-2 was replaced with (5-bromo-6- chloropyridin-3- yl)methanol |

### Example 74: Synthesis of 5-(2-chloro-5-(hydroxymethyl)pyridin-3-yl)-2-(3-(difluoromethoxy)-4-fluorobenzyl)-7-((2-(methylamino)-1H-imidazol-1-yl)methyl)-3,4-dihydroisoquinolin-1(2H)-one (74)

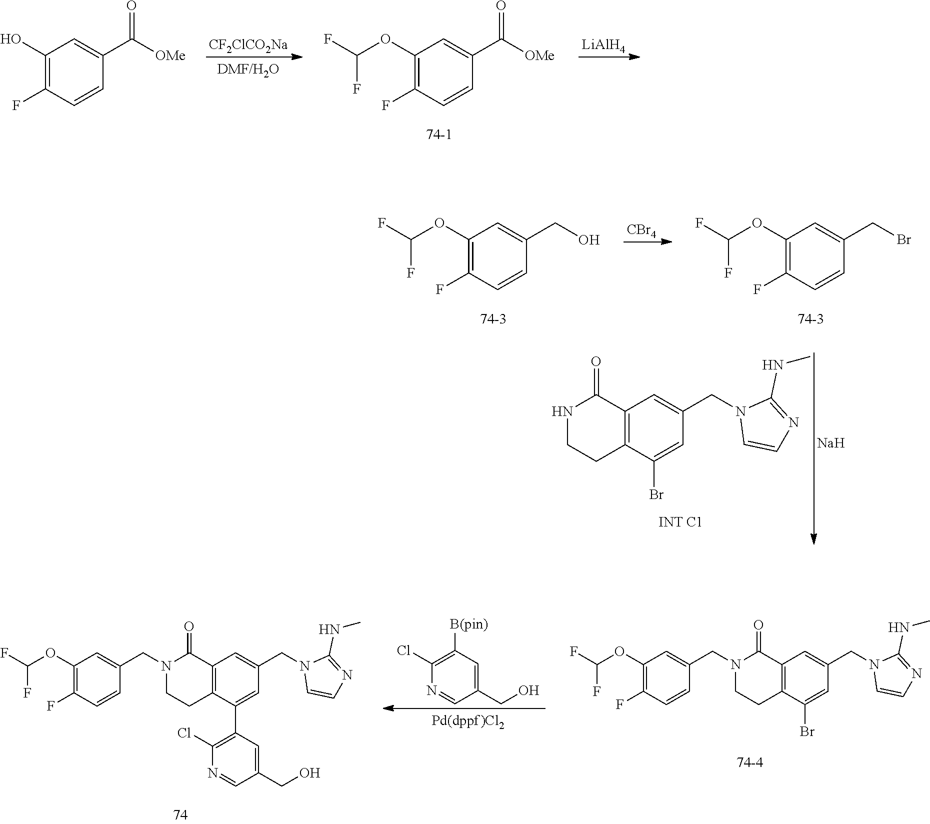

Step 1: A mixture of methyl 4-fluoro-3-hydroxybenzoate (4.0 g, 23.5 mmol, 1.0 eq), sodium 2-chloro-2,2-difluoroacetate (8.9 g, 58.75 mmol, 2.5 eq) and Cs₂CO₃ (11.5 g, 32.25 mmol, 1.5 eq) in DMF/H₂O (10 mL/1 mL) was stirred at 100° C. for 6 hours. The mixture was poured into water (500 mL), extracted with ethyl acetate (50 mL×3). The combined organic layers were dried and concentrated, then purified by flash column (0%˜7% EtOAc in hexane) to afford methyl 3-(difluoromethoxy)-4-fluorobenzoate (74-1) (4.2 g, 81% yield) as a colourless oil. ¹H NMR (CD₃OD 400 MHz) δ 7.96-7.89 (m, 2H), 7.37 (dd, J=10.0, 8.8 Hz, 1H), 6.93 (t, J=33.2 Hz, 1H), 3.91 (s, 3H).

Step 2: To a solution of compound 74-1 (4.2 g, 19.1 mmol, 1.0 eq) in THF (40 mL) was added LiAlH₄ (870 mg, 22.9 mmol, 1.2 eq) at 0° C. under nitrogen atmosphere. The mixture was stirred at 15° C. for 16 hrs. The mixture was quenched by 10% aqueous NaOH (0.8 mL). The suspension was filtered and the filtrate washed with tetrahydrofuran (20 mL×3). The combined organic phase was dried and concentrated, then purified by flash column (50% of EtOAc in Hexane) to afford (3-(difluoromethoxy)-4-fluorophenyl)methanol (74-2) (2.8 g, 76% yield) as a colourless oil. ¹H NMR (CD₃OD 400 MHz) δ 7.29 (d, J=7.6 Hz, 2H), 7.25-7.15 (m, 2H), 6.82 (t, J=33.2 Hz, 1H), 4.57 (s, 2H).

Step 3: A solution of compound 74-2 (1 g, 5.20 mmol) and PPh₃ (1.64 g, 6.25 mmol) in DCM (15 mL) was cooled to 0° C. CBr₄ (1.9 g, 5.72 mmol) in DCM (15 mL) was added dropwise, the resulting mixture was warmed to 2-13° C. for 5 hours. The solution was concentrated and purified by flash column (10%˜20% EtOAc in Hexane) to afford 4-(bromomethyl)-2-(difluoromethoxy)-1-fluorobenzene (74-3) (1.2 g, 92% yield) as a colorless oil. ¹H NMR (CD₃OD 400 MHz) δ 7.28 (d, J=1.6 Hz, 1H), 7.27-7.21 (m, 1H), 7.14-7.09 (m, 1H), 6.75 (t, J=73.2 Hz, 1H), 4.45 (s, 2H).

Step 4: 5-bromo-2-(3-(difluoromethoxy)-4-fluorobenzyl)-7-((2-(methylamino)-1H-imidazol-1-yl)methyl)-3,4-dihydroisoquinolin-1(2H)-one (74-4) was synthesized from compound 74-3 and intermediate (A1) using general procedure for NaH mediated SN2 reaction described in Example 1. ¹H NMR (CD₃OD 400 MHz) δ 7.86 (d, J=1.2 Hz, 1H), 7.56 (d, J=1.6 Hz, 1H), 7.32-7.25 (m, 3H), 7.25-6.67 (m, 3H), 5.03 (s, 2H), 4.76 (s, 2H), 3.60 (t, J=6.8 Hz, 2H), 3.08 (t, J=6.4 Hz, 2H), 2.90 (s, 3H). LCMS: MS m/z 510.9 [M+H]⁺.

Step 4: 5-(2-chloro-5-(hydroxymethyl)pyridin-3-yl)-2-(3-(difluoromethoxy)-4-fluorobenzyl)-7-((2-(methylamino)-1H-imidazol-1-yl)methyl)-3,4-dihydroisoquinolin-1(2H)-one (74) was synthesized from compound 74-4 and (5-bromo-6-chloropyridin-3-yl)methanol using the general procedure for Suzuki coupling described in step 3 of Example 52. ¹H NMR (400 MHz, Methanol-d₄) δ ppm 8.44 (d, J=2.4 Hz, 1H), 7.97 (d, J=1.6 Hz, 1H), 7.78 (d, J=2.0 Hz, 1H), 7.37 (d, J=2.0 Hz, 1H), 7.27-7.24 (m, 3H), 7.02-6.66 (m, 3H), 5.20 (s, 2H), 4.91-4.76 (m, 2H), 4.72 (d, J=6.8 Hz, 2H), 3.56-3.52 (m, 2H), 3.03 (s, 3H), 2.79-2.73 (m, 2H). LC-MS: [M+H]⁺=572.3.

### Example 75 and Example 76: Synthesis of (R)-5-(2-chloro-5-(1-hydroxyethyl)pyridin-3-yl)-2-(4-fluoro-3-methoxybenzyl)-7-((2-(methylamino)-1H-imidazol-1-yl)methyl)-3,4-dihydroisoquinolin-1(2H)-one (75) and (S)-5-(2-chloro-5-(1-hydroxyethyl)pyridin-3-yl)-2-(4-fluoro-3-methoxybenzyl)-7-((2-(methylamino)-1H-imidazol-1-yl)methyl)-3,4-dihydroisoquinolin-1(2H)-one (76)

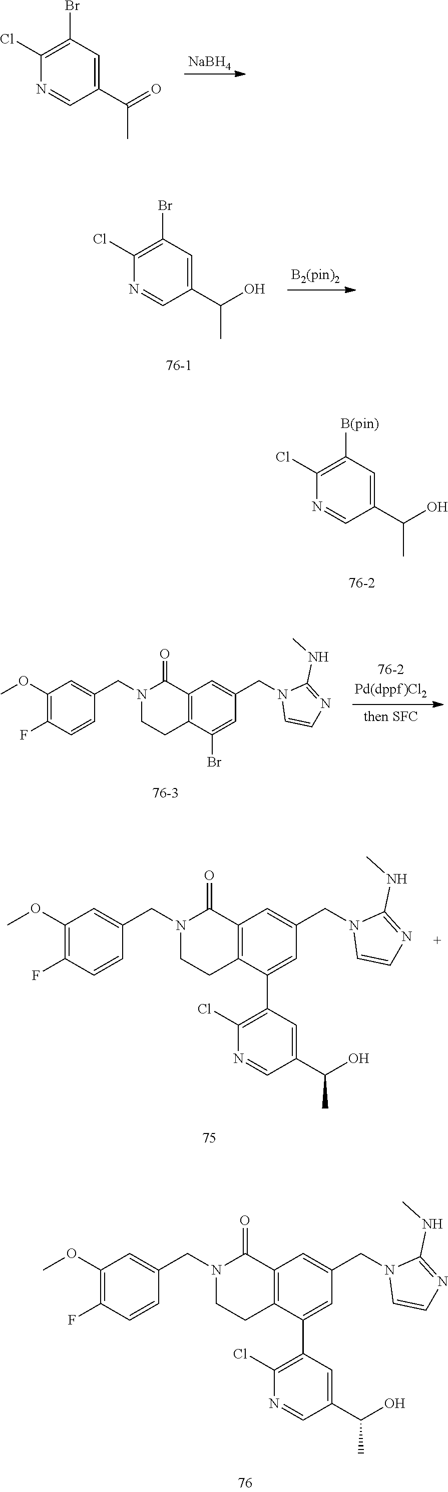

Step 1: To the solution of 1-(5-bromo-6-chloropyridin-3-yl)ethan-1-one (960 mg, 4.09 mmol) in THF (15 mL) and EtOH (15.00 mL) was added NaBH₄ (465 mg, 12.28 mmol) and LiCl (521 mg, 12.28 mmol). Then the mixture was stirred at 25° C. for 2.5 h. The mixture was filtered. The filtrate was concentrated. To the residue was added 50 mL of water, and extracted with EtOAc (2×300 mL). The organic layers were collected, dried and concentrated to give 1-(5-bromo-6-chloropyridin-3-yl)ethan-1-ol (76-1) (900 mg, 93%) as light yellow oil. LCMS: MS m/z 235.9 [M+H]⁺.

Step 2: Potassium acetate (415 mg, 4.23 mmol) was added to the solution of 76-1 (500 mg, 2.114 mmol) and 4,4,4′,4′,5,5,5′,5′-octamethyl-2,2′-bi(1,3,2-dioxaborolane) (805 mg, 3.17 mmol) in dioxane (15 mL). The mixture was degassed for 10 min, followed by the addition of PdCl2(dppf).CH₂Cl₂ (104 mg, 0.127 mmol). The mixture was stirred at 90° C. for 1.5 h under nitrogen atmosphere. Then the reaction mixture was filtered. The filtrate was concentrated and purified by flash column chromatography on silica gel (20˜70% EtOAc in Hexanes) to give 76-2 (566 mg, 80%) as colorless oil. LCMS: MS m/z 202.1 [M+H]⁺.

Step 3: To a reaction tube were added 5-bromo-2-(4-fluoro-3-methoxybenzyl)-7-((2-(methylamino)-1H-imidazol-1-yl)methyl)-3,4-dihydroisoquinolin-1(2H)-one (76-3) (50 mg, 0.106 mmol), compound 76-2 (74.9 mg, 0.264 mmol), PdCl₂(dppf).CH₂Cl₂ (8.63 mg, 10.56 μmol), Na₂CO₃ (44.8 mg, 0.423 mmol) and solvents dioxane (6 mL)/water (0.6 mL). Then the tube was heated and stirred at 90° C. for 2 hrs. under nitrogen atmosphere. The reaction mixture was filtered, the filtrate was concentrated and purified by preparative-HPLC to give a racemic mixture as a white solid. The racemic mixture was separated by SFC (Column: CC4, Condition: EtOH+0.5% NH₄OH) to give example 75 (first peak) and example 76 (second peak).

Example 75: ¹H NMR (400 MHz, Methanol-d₄) δ ppm 8.41 (s, 1H), 7.97 (s, 1H), 7.73 (d, J=2.2 Hz, 1H), 7.17 (s, 1H), 7.08-6.96 (m, 2H), 6.92-6.84 (m, 1H), 6.66 (s, 1H), 6.59 (s, 1H), 5.04 (s, 2H), 4.95-4.89 (m, 1H), 4.82-4.61 (m, 2H), 3.84 (s, 3H), 3.48 (t, J=6.4 Hz, 2H), 2.89-2.76 (m, 3H), 2.76-2.61 (m, 2H), 1.47 (d, J=6.5 Hz, 3H). LC-MS: [M+H]⁺=549.9.
Example 76: ¹H NMR (400 MHz, Methanol-d₄) δ ppm 8.41 (s, 1H), 7.97 (s, 1H), 7.74 (d, J=2.2 Hz, 1H), 7.18 (s, 1H), 7.12-6.99 (m, 2H), 6.89 (s, 1H), 6.68 (s, 1H), 6.61 (s, 1H), 5.05 (s, 2H), 4.92 (q, J=6.5 Hz, 1H), 4.81-4.60 (m, 2H), 3.84 (s, 3H), 3.48 (t, J=6.4 Hz, 2H), 2.86 (s, 3H), 2.77-2.59 (m, 2H), 1.47 (d, J=6.5 Hz, 3H). LC-MS: [M+H]*=549.9.
Note: 5-bromo-2-(4-fluoro-3-methoxybenzyl)-7-((2-(methylamino)-1H-imidazol-1-yl)methyl)-3,4-dihydroisoquinolin-1(2H)-one (76-3) was synthesized from 4-(bromomethyl)-1-fluoro-2-methoxybenzene and intermediate (A1) using the general procedure for NaH mediated SN2 reaction described in Example 1. LC-MS: [M+H]⁺=473.0.

### Example 77: Synthesis of 4-(2-(4-fluoro-3-methoxybenzyl)-7-((2-(methylamino)-1H-imidazol-1-yl)methyl)-1-oxo-1,2,3,4-tetrahydroisoquinolin-5-yl)-1-methyl-1H-pyrazole-3-carbonitrile (77)

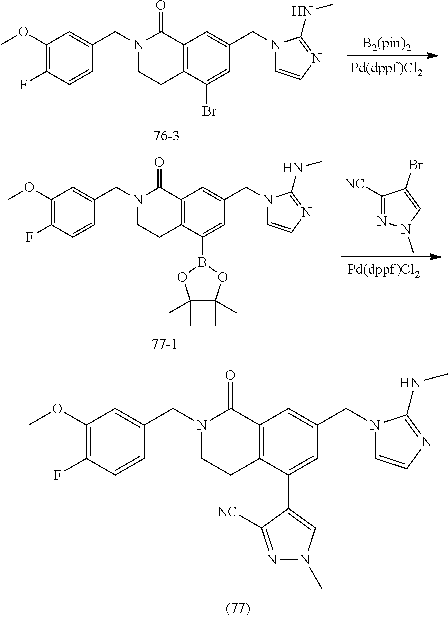

Step 1: To a reaction tube was added 5-bromo-2-(4-fluoro-3-methoxybenzyl)-7-((2-(methylamino)-1H-imidazol-1-yl)methyl)-3,4-dihydroisoquinolin-1(2H)-one (76-3) (40 mg, 0.085 mmol), 4,4,4′,4′,5,5,5′,5′-octamethyl-2,2′-bi(1,3,2-dioxaborolane) (64.4 mg, 0.254 mmol), potassium acetate (24.88 mg, 0.254 mmol) and PdCl₂(dppf)-CH₂Cl₂ (6.90 mg, 8.45 μmol). Then under N₂, 1,4-dioxane (3.0 mL) was added, and the mixture heated to 110° C. and stirred for 20 hrs. Then the reaction was filtered and the filtrate was concentrated to give crude 2-(4-fluoro-3-methoxybenzyl)-7-((2-(methylamino)-1H-imidazol-1-yl)methyl)-5-(4,4,5,5-tetramethyl-1,3,2-dioxaborolan-2-yl)-3,4-dihydroisoquinolin-1(2H)-one (77-1). LC-MS: [M+H]⁺=521.3.

Step 2: To a reaction tube was added 4-bromo-1-methyl-1H-pyrazole-3-carbonitrile (7.0 mg, 0.038 mmol), Na₂CO₃ (11.97 mg, 0.113 mmol) and PdCl₂(dppf)-CH₂Cl₂ (3.07 mg, 3.76 μmol). Then under N₂, the crude compound 77-1 (20.58 mg, 0.040 mmol) in 1,4-dioxane (1.4 mL) was added and then water (0.14 mL) was added. The mixture was heated to 90° C. and stirred for 16 hrs. Then DMSO (5 mL) was added, the mixture was filtered and the filtrate was purified by HPLC to give 4-(2-(4-fluoro-3-methoxybenzyl)-7-((2-(methylamino)-1H-imidazol-1-yl)methyl)-1-oxo-1,2,3,4-tetrahydroisoquinolin-5-yl)-1-methyl-1H-pyrazole-3-carbonitrile (77) (5.5 mg, 23.8% yield). ¹H NMR (400 MHz, Methanol-d₄) δ ppm 7.97 (d, J=1.8 Hz, 1H), 7.95 (s, 1H), 7.41 (d, J=1.9 Hz, 1H), 7.11-7.02 (m, 2H), 6.99 (q, J=2.5 Hz, 2H), 6.90 (dq, J=6.2, 2.1 Hz, 1H), 5.19 (s, 2H), 4.75 (s, 2H), 4.01 (s, 3H), 3.85 (s, 3H), 3.52 (t, J=6.6 Hz, 2H), 2.99 (d, J=4.7 Hz, 5H). LC-MS: [M+H]*=500.3.

### Example 78: Synthesis of (S)-5-cyclopropyl-2-(1-(6-fluoro-5-methoxypyridin-3-yl)ethyl)-7-((2-(methylamino)-1H-imidazol-1-yl)methyl)-3,4-dihydroisoquinolin-1(2H)-one (78)

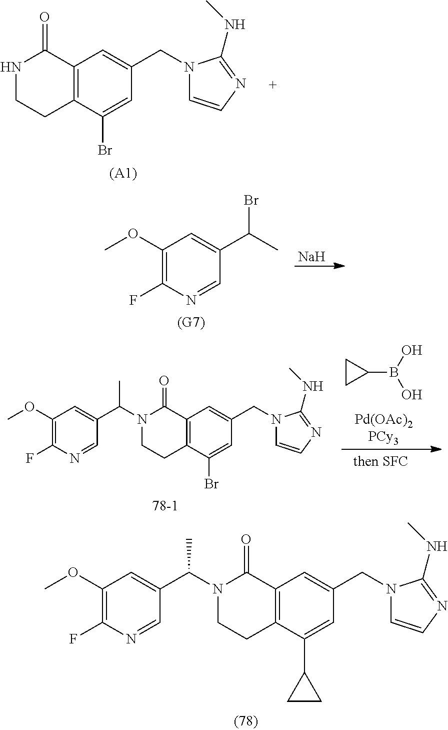

Step 1: 5-bromo-2-(1-(6-fluoro-5-methoxypyridin-3-yl)ethyl)-7-((2-(methylamino)-1H-imidazol-1-yl)methyl)-3,4-dihydroisoquinolin-1(2H)-one (78-1) was synthesized from intermediate (G7) and 4-(bromomethyl)-1-fluoro-2-methoxybenzene and intermediate (A1) using the general procedure for NaH mediated SN2 reaction described in Example 1. ¹H NMR (400 MHz, Methanol-d₄) δ 7.82 (s, 1H), 7.76-7.69 (m, 2H), 7.49 (dd, J=9.7, 1.9 Hz, 1H), 7.05-6.93 (m, 2H), 6.03 (q, J=7.1 Hz, 1H), 5.11 (s, 2H), 3.90 (s, 3H), 3.64-3.54 (m, 1H), 3.14-2.89 (m, 6H), 1.66 (d, J=7.2 Hz, 3H). LCMS: [M+H]⁺=487.8.

Step 2: A mixture of compound 78-1 (30 mg, 0.061 mmol), cyclopropylboronic acid (10.55 mg, 0.123 mmol), Pd(OAc)₂ (2.76 mg, 0.012 mmol), tripotassium phosphate (45.6 mg, 0.215 mmol) and tricyclohexylphosphine (6.89 mg, 0.025 mmol) in toluene (1 mL)/Water (0.05 mL) was heated and stirred at 110° C. for 18 hrs under nitrogen atmosphere. The reaction mixture was cooled to room temperature and concentrated. The residue was purified by preparative-HPLC to give a racemic mixture as a yellow solid. The racemic mixture was separated by chiral SFC (Column: (R,R)-WHELKO1, 5 um, 4.6 mm*250 mm, Condition: EtOH+0.5% NH₄OH) to give (S)-5-cyclopropyl-2-(1-(6-fluoro-5-methoxypyridin-3-yl)ethyl)-7-((2-(methylamino)-1H-imidazol-1-yl)methyl)-3,4-dihydroisoquinolin-1(2H)-one (78) (the 2nd peak on the prep SFC) together with its enantiomer. ¹H NMR (400 MHz, Methanol-d₄) δ 7.73 (s, 1H), 7.71-7.66 (m, 1H), 7.49 (dd, J=9.7, 1.9 Hz, 1H), 7.04-6.97 (m, 1H), 6.65-6.56 (m, 2H), 6.07 (q, J=7.1 Hz, 1H), 4.93 (s, 2H), 3.89 (s, 3H), 3.55 (ddd, J=12.6, 9.3, 4.7 Hz, 1H), 3.24 (dd, J=7.1, 5.3 Hz, 1H), 3.17-3.09 (m, 1H), 3.02-2.90 (m, 1H), 2.85 (s, 3H), 2.66 (s, 1H), 1.90 (td, J=9.3, 8.8, 4.3 Hz, 1H), 1.66 (d, J=7.2 Hz, 3H), 0.94 (ddd, J=8.5, 3.6, 1.9 Hz, 2H), 0.62-0.53 (m, 2H). LCMS: [M+H]⁺=450.2.

Following a similar procedure to that of Example 78 (unless otherwise specified). The following compounds were prepared from their corresponding precursors.

| Ex. | Name/Structure | ¹HNMR/LC-MS | Precursors |
|---|---|---|---|
| 79 | 5-cyclopropyl-2-((6-fluoro-5- | ¹H NMR (400 MHz, Methanol-d₄) δ | Step 1: |
| | methoxypyridin-3-yl)methyl)-7-((2- | ppm 7.69 (s, 2H), 7.54 (dd, J = 9.8, | intermediate (G7) |
| | (methylamino)-1H-imidazol-1- | 1.9 Hz, 1H), 7.03 (s, 1H), 6.64 (d, | was replaced with |
| | yl)methyl)-3,4-dihydroisoquinolin- | J = 1.8 Hz, 2H), 4.93 (s, 2H), 4.77 (s, | intermediate (G8) |
| | 1(2H)-one | 2H), 3.89 (s, 3H), 3.61 (t, J = 6.7 | |
| | 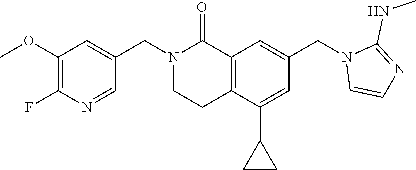 | Hz, 2H), 3.14 (t, J = 6.7 Hz, 2H), 2.86 (s, 3H), 1.97-1.85 (m, 1H), 1.01-0.92 (m, 2H), 0.65-0.54 (m, 2H). LC-MS: (ESI) m/z 436.1 [M + H]⁺ | |
| 80 | (S)-5-(1-(5-cyclopropyl-7-((2- | ¹H NMR (400 MHz, Methanol-d₄) δ | Step 1: |
| | (methylamino)-1H-imidazol-1- | ppm 8.28 (d, J = 1.2 Hz, 1H), 7.68 | intermediate (G7) |
| | yl)methyl)-1-oxo-3,4- | (s, 1H), 7.58 (s, 1H), 7.01 (s, 1H), | was replaced with |
| | dihydroisoquinolin-2(1H)-yl)ethyl)- | 6.58 (dd, J = 7.4, 1.5 Hz, 2H), 6.07 | intermediate (G3) |
| | 3-ethoxypicolinonitrile | (q, J = 7.1 Hz, 1H), 4.91 (s, 2H), | |
| | 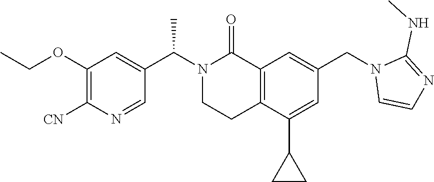 | 4.26 (qd, J = 7.0, 2.3 Hz, 2H), 3.62- 3.52 (m, 1H), 3.20-3.10 (m, 1H), 3.01 (dd, J = 9.5, 5.4 Hz, 1H), 2.84 (s, 3H), 2.65 (s, 3H), 1.90 (s, 1H), 1.69 (d, J = 7.2 Hz, 3H), 1.46 (t, J = 7.0 Hz, 3H), 0.99-0.92 (m, 2H), 0.61-0.51 (m, 2H). LC-MS: (ESI) m/z 471.0 [M + H]⁺ | |
| 81 | (S)-5-cyclopropyl-2-(1-(4-ethoxy-5- | ¹H NMR (400 MHz, Methanol-d₄) δ | Step 1: |
| | fluoropyridin-2-yl)ethyl)-7-((2- | ppm 8.24 (d, J = 3.3 Hz, 1H), 7.66 | intermediate (G7) |
| | (methylamino)-1H-imidazol-1- | (s, 1H), 7.13 (d, J = 6.9 Hz, 1H), | was replaced with |
| | yl)methyl)-3,4-dihydroisoquinolin- | 7.00 (s, 1H), 6.62 (d, J = 2.3 Hz, | intermediate (G5) |
| | 1(2H)-one | 2H), 5.96 (q, J = 7.0 Hz, 1H), 4.92 | |
| | 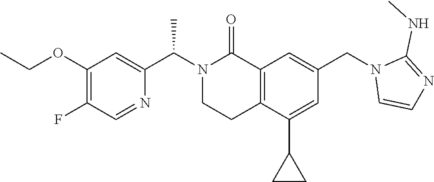 [SMILES: CCOc1cc([C@H](C)N2CCc3c(cc(Cn4ccnc4NC)cc3C3CC3)C2=O)ncc1F] | (s, 2H), 4.19 (qd, J = 7.0, 2.8 Hz, 2H), 3.57 (ddd, J = 13.9, 9.2, 4.9 Hz, 1H), 3.40 (ddd, J = 12.5, 7.1, 5.2 Hz, 1H), 3.14-3.04 (m, 1H), 3.02-2.91 (m, 1H), 2.85 (s, 3H), 1.97-1.84 (m, 1H), 1.62 (d, J = 7.1 Hz, 3H), 1.43 (t, J = 7.0 Hz, 3H), 1.00-0.88 (m, 2H), 0.57 (dd, J = 5.4, 1.6 Hz, 2H). LC-MS: (ESI) m/z 464.3 [M + H]⁺ | |
| 82 | (R)-6-(1-(5-cyclopropyl-7-((2- | ¹H NMR (400 MHz, Methanol-d₄) δ | Step 1: |
| | (methylamino)-1H-imidazol-1- | ppm 8.63 (s, 1H), 7.65 (s, 1H), 7.17 | intermediate (G7) |
| | yl)methyl)-1-oxo-3,4- | (s, 1H), 7.01 (s, 1H), 6.73-6.51 | was replaced with |
| | dihydroisoquinolin-2(1H)-yl)ethyl)- | (m, 2H), 5.95 (q, J = 7.0 Hz, 1H), | intermediate (G1) |
| | 4-ethoxynicotinonitrile | 4.29 (q, J = 6.9 Hz, 2H), 3.63 (td, | |
| | 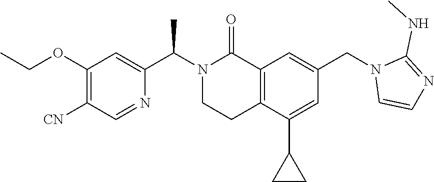 | J = 9.0, 4.6 Hz, 1H), 3.59-3.47 (m, 1H), 3.17-3.09 (m, 1H), 3.09- 2.97 (m, 1H), 2.84 (s, 3H), 1.99- 1.86 (m, 1H), 1.66 (d, J = 7.1 Hz, 3H), 1.46 (t, J = 7.0 Hz, 3H), 0.96 (dd, J = 7.3, 3.4 Hz, 2H), 0.67- 0.49 (m, 2H). LC-MS: (ESI) m/z 471.0 [M + H]⁺ | |
| 83 | (S)-6-(1-(5-cyclopropyl-7-((2- | ¹H NMR (400 MHz, Methanol-d₄) δ | Step 1: |
| | (methylamino)-1H-imidazol-1- | ppm 8.63 (s, 1H), 7.65 (d, J = 1.8 | intermediate (G7) |
| | yl)methyl)-1-oxo-3,4- | Hz, 1H), 7.17 (s, 1H), 7.00 (d, J = | was replaced with |
| | dihydroisoquinolin-2(1H)-yl)ethyl)- | 1.8 Hz, 1H), 6.66-6.57 (m, 2H), | intermediate (G1) |
| | 4-ethoxynicotinonitrile | 5.95 (q, J = 7.1 Hz, 1H), 4.93 (s, | |
| | 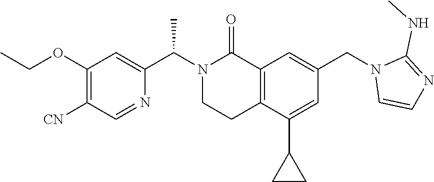 | 2H), 4.36-4.24 (m, 2H), 3.57 (dddd, J = 39.4, 12.6, 8.1, 5.2 Hz, 2H), 3.18-3.00 (m, 2H), 2.84 (s, 3H), 1.91 (ddd, J = 13.8, 8.6, 5.3 Hz, 1H), 1.66 (d, J = 7.1 Hz, 3H), 1.46 (t, J = 7.0 Hz, 3H), 1.02-0.86 (m, 3H), 0.58 (tdd, J = 7.1, 5.9, 4.7, 2.8 Hz, 2H). LC-MS: (ESI) m/z 471.3 [M + H]⁺ | |
| 84 | 5-((5-cyclopropyl-7-((2- | ¹H NMR (400 MHz, Methanol-d4) δ | Step 1: |
| | (methylamino)-1H-imidazol-1- | 8.24 (d, J = 1.7 Hz, 1H), 7.69 (d, J = | intermediate (G7) |
| | yl)methyl)-1-oxo-3,4- | 1.9 Hz, 1H), 7.61 (d, J = 1.6 Hz, | was replaced with |
| | dihydroisoquinolin-2(1H)- | 1H), 7.02 (d, J = 1.9 Hz, 1H), 6.64- | intermediate (G4) |
| | yl)methyl)-3-ethoxypicolinonitrile | 6.53 (m, 2H), 4.85 (s, 2H), 4.24 (d, | |
| | 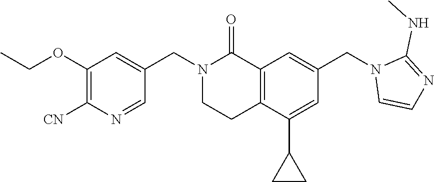 | J = 7.0 Hz, 2H), 3.65 (t, J = 6.7 Hz, 2H), 3.17 (t, J = 6.7 Hz, 2H), 2.84 (s, 3H), 1.97-1.89 (m, 1H), 1.46 (t, J = 7.0 Hz, 3H), 1.01-0.93 (m, 2H), 0.59 (dd, J = 5.4, 1.8 Hz, 2H). LC-MS: (ESI) m/z 457.0 [M + H]⁺ | |
| 85 | (S)-5-cyclopropyl-2-(1-(5-fluoro-4- | ¹H NMR (400 MHz, Methanol-d₄) δ | Step 1: |
| | methoxypyridin-2-yl)ethyl)-7-((2- | ppm 8.24 (d, J = 3.1 Hz, 1H), 7.67 | intermediate (G7) |
| | (methylamino)-1H-imidazol-1- | (s, 1H), 7.16 (d, J = 6.9 Hz, 1H), | was replaced with |
| | yl)methyl)-3,4-dihydroisoquinolin- | 7.00 (s, 1H), 6.60 (d, J = 3.5 Hz, | intermediate (G9) |
| | 1(2H)-one | 2H), 5.98 (q, J = 6.9 Hz, 1H), 4.92 | |
| | 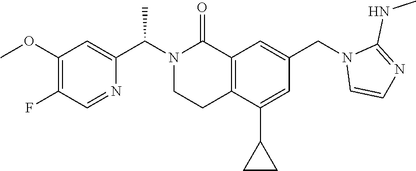 | (s, 2H), 3.94 (s, 3H), 3.56 (td, J = 9.0, 4.7 Hz, 1H), 3.41 (dt, J = 12.5 5.9 Hz, 1H), 3.19-3.02 (m, 1H), 3.00 (dd, J = 9.3, 5.5 Hz, 1H), 2.85 (s, 3H), 1.90 (s, 1H), 1.63 (d, J = 7.1 Hz, 3H), 0.98-0.90 (m, 2H), 0.65-0.51 (m, 2H). LC-MS: (ESI) m/z 450.0 [M + H]⁺ | |

### Example 86: Synthesis of 5-cyclopropyl-2-((1-ethyl-5-methoxy-1H-pyrazol-3-yl)methyl)-7-((2-(methylamino)-1H-imidazol-1-yl)methyl)-3,4-dihydroisoquinolin-1(2H)-one (86)

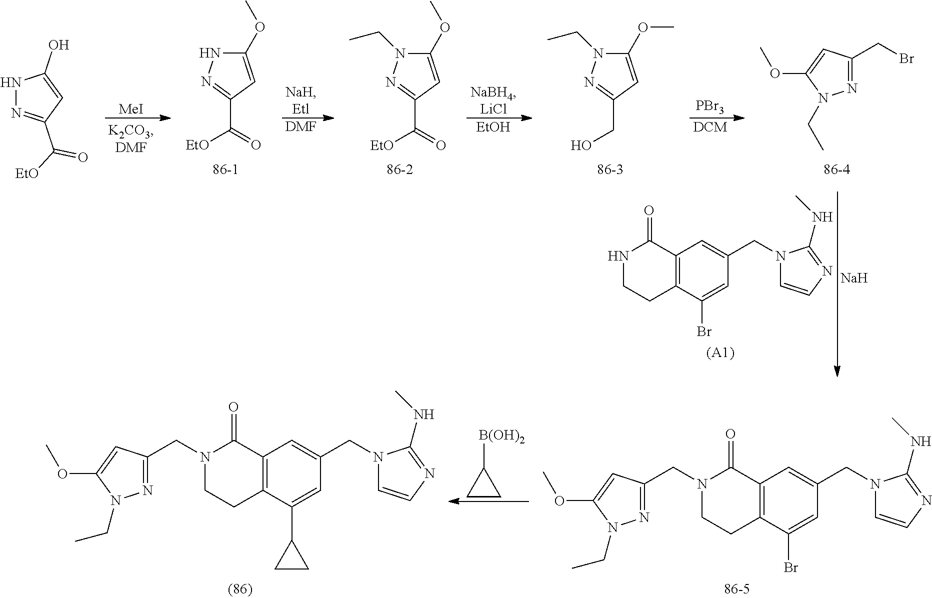

Step 1: To the suspension of ethyl 5-hydroxy-1H-pyrazole-3-carboxylate (4.5 g, 28.82 mmol) and K₂CO₃ (4.77 g, 34.58 mmol) was added MeI (4.1 g, 28.82 mmol) at 0° C. Then the reaction was stirred at 25° C. for 16 hrs. The reaction mixture was diluted with water (50 mL) and brine (50 mL). The aqueous layer was extracted with ethyl acetate (50 mL×3). The combined organic layers were dried and concentrated. The residue was purified by Prep-HPLC to afford ethyl 5-methoxy-1H-pyrazole-3-carboxylate (86-1) (900 mg, 18.3% yield) as a white solid. ¹H NMR (DMSO-d6 400 MHz): δ 13.10 (br s, 1H), 6.32-5.98 (m, 1H), 4.26 (br d, J=5.7 Hz, 2H), 3.79 (br s, 3H), 1.43-1.04 (m, 1H), 1.43-1.04 (m, 3H). LCMS: MS (ESI) m/z 170.9 [M+H]⁺.

Step 2: To a solution of compound 86-1 (700 mg, 4.1 mmol) in DMF (10 mL) was added NaH (328 mg, 8.2 mmol, 60% in oil) at 0° C. under N₂ atmosphere, the mixture was then stirred at 0° C. for 20 mins. Then Etl (1.28 g, 8.2 mmol) was added at 0° C. The resulting mixture was stirred at 20° C. for 30 mins. The reaction was quenched by H₂O (50 mL) and extracted with ethyl acetate. The combined organic layers were dried and concentrated. The residue was purified by column chromatography (10˜33% of EtOAc in Hexane) to afford ethyl 1-ethyl-5-methoxy-1H-pyrazole-3-carboxylate (86-2) (170 mg, 20.9% yield) as a colorless oil. ¹H NMR (CDCl₃ 400 MHz): □ 6.07 (s, 1H), 4.39 (q, J=7.2 Hz, 2H), 4.09 (q, J=7.2 Hz, 2H), 3.92 (s, 3H), 1.39 (dt, J=2.4, 7.2 Hz, 6H). LCMS: MS (ESI) m/z 198.9 [M+H]⁺.

Step 3: To a solution of compound 86-2 (140 mg, 0.7 mmol) in EtOH (3 mL) was added LiCl (148.4 mg, 3.5 mmol) and NaBH₄ (132.4 mg, 3.5 mmol) at 0° C., the mixture was then stirred at 20° C. for 1 h. The reaction was quenched by H₂O (30 mL) and extracted with ethyl acetate. The combined organic layers were dried and concentrated. The residue was and purified by column chromatography (16˜50% of EtOAc in Hexane) to afford (1-ethyl-5-methoxy-1H-pyrazol-3-yl)methanol (86-3) (140 mg, 68.8%) as a colorless oil. LCMS: MS (ESI) m/z 157.0 [M+H]⁺.

Step 4: To a solution of compound 86-3 (110 mg, 0.7 mmol) in DCM (20 mL) was added PBr₃ (305.9 mg, 1.13 mmol) at 0° C., and the mixture was then stirred at 20° C. for 30 mins. The reaction was quenched by aq.NaHCO₃ (20 mL) and extracted with DCM (20 mL×2). The combined organic layer was dried and concentrated to afford 3-(bromomethyl)-1-ethyl-5-methoxy-1H-pyrazole (86-4) (76 mg, crude) as a light yellow oil. LCMS: MS (ESI) m/z 218.8 [M+H]⁺.

Step 5: 5-bromo-2-((1-ethyl-5-methoxy-1H-pyrazol-3-yl)methyl)-7-((2-(methylamino)-1H-imidazol-1-yl)methyl)-3,4-dihydroisoquinolin-1(2H)-one (86-5) was synthesized from intermediate (A1) and compound 86-4 using the general procedure for NaH mediated SN2 reaction described in Example 1. LCMS: MS (ESI) m/z 473.1 [M+H]⁺.

Step 6: 5-cyclopropyl-2-((1-ethyl-5-methoxy-1H-pyrazol-3-yl)methyl)-7-((2-(methylamino)-1H-imidazol-1-yl)methyl)-3,4-dihydroisoquinolin-1(2H)-one (86) was synthesized from 86-5 following the general procedure for coupling of cyclopropylboronic acid described in Example 78. ¹H NMR (CD₃OD 400 MHz) δ=8.54 (br s, 1H), 7.67 (d, J=1.2 Hz, 1H), 7.08 (s, 1H), 6.76 (dd, J=2.0, 8.0 Hz, 2H), 5.62 (s, 1H), 4.98 (s, 2H), 4.63 (s, 2H), 3.95 (q, J=7.2 Hz, 2H), 3.87 (s, 3H), 3.58 (t, J=6.7 Hz, 2H), 3.13 (t, J=6.7 Hz, 2H), 2.92 (s, 3H), 1.98-1.88 (m, 1H), 1.30 (t, J=7.2 Hz, 3H), 1.01-0.92 (m, 2H), 0.65-0.57 (m, 2H). LCMS: [M+H]⁺=435.1.

### Example 87: Synthesis of 5-cyclopropyl-2-((5-ethoxy-1-methyl-1H-pyrazol-3-yl)methyl)-7-((2-(methylamino)-1H-imidazol-1-yl)methyl)-3,4-dihydroisoquinolin-1(2H)-one (87)

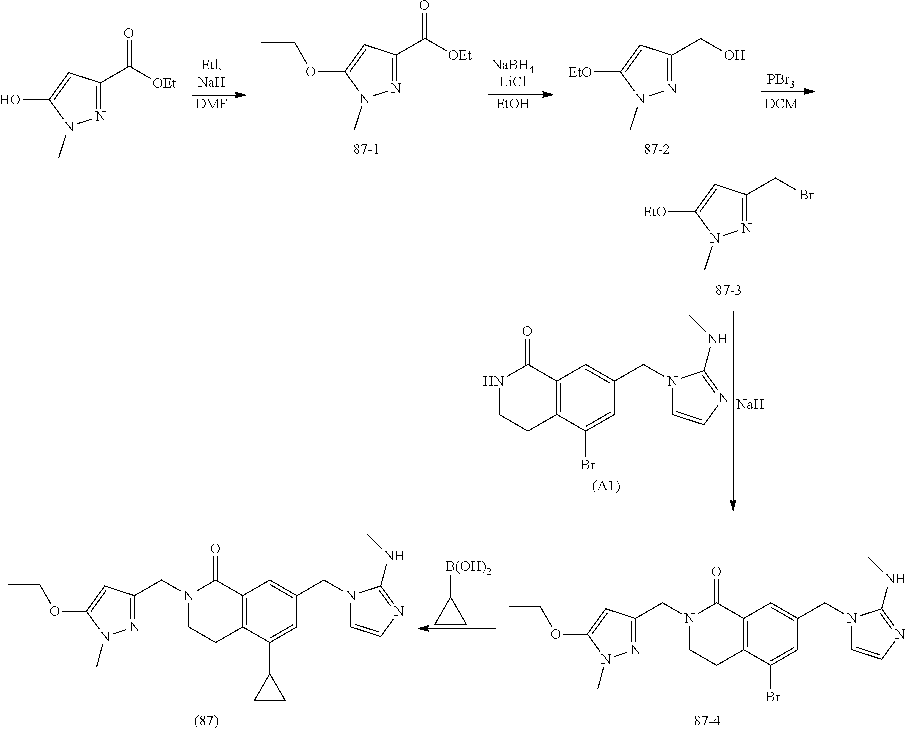

Step 1: To a solution of ethyl 5-hydroxy-1-methyl-1H-pyrazole-3-carboxylate (1.0 g, 5.9 mmol) in DMF (13 mL) was added K₂CO₃ (4.87 g, 35.3 mmol) and Etl (6.23 g, 40.0 mmol) at 0° C. under nitrogen atmosphere. The reaction mixture was stirred at 25° C. for 36 hours. Water (20 mL) was added. The resulting mixture was extracted with EtOAc (10 mL×3). The organic phase was concentrated and purified by silica gel chromatography (Hexane/EtOAc=1:1) to afford ethyl 5-ethoxy-1-methyl-1H-pyrazole-3-carboxylate (87-1) (800 mg, 68.5%) as a yellow oil, LCMS: MS (ESI) m/z 199.1 [M+H]⁺.

Step 2: To a solution of compound 87-1 (400 mg, 2.02 mmol) in ethanol (10 mL) was added LiCl (423 mg, 10.1 mmol) and NaBH₄ (384 mg, 10.1 mmol) at 0° C. under nitrogen atmosphere. The reaction mixture was stirred at 25° C. for 24 hours. 4M aq. HCl (2 mL) was added. The resulting mixture was adjusted to pH˜8 by sat. NaHCO₃. The resulting mixture was extracted with EtOAc (20 mL×3). The combined organic phase was concentrated to afford (5-ethoxy-1-methyl-1H-pyrazol-3-yl)methanol (87-2) (300 mg, crude) as a yellow oil. LCMS: MS (ESI) m/z 157.0 [M+H]⁺.

Step 3: To a solution of compound 87-2 (0.21 g, 1.35 mmol) in dichloromethane (20 mL) was added PBr₃ (0.2 mL, 2.15 mmol) at 0° C. under nitrogen atmosphere. The reaction mixture was stirred at 25° C. for 2 hours. The reaction mixture was adjusted to pH-8 by NH₃H₂O (15%). The resulting mixture was extracted with EtOAc (20 mL×3), dried and concentrated to afford 3-(bromomethyl)-5-ethoxy-1-methyl-1H-pyrazole (87-3) (200 mg, crude). LCMS: MS (ESI) m/z 219.1 and 221.1 [M+H]⁺.

Step 3: 5-bromo-2-((5-ethoxy-1-methyl-1H-pyrazol-3-yl)methyl)-7-((2-(methylamino)-1H-imidazol-1-yl)methyl)-3,4-dihydroisoquinolin-1(2H)-one (87-4) was synthesized from intermediate (A1) and compound 87-3 using the general procedure for NaH mediated SN2 reaction described in Example 1. LCMS: MS (ESI) m/z 473.1 [M+H]⁺.

Step 6: 5-cyclopropyl-2-((5-ethoxy-1-methyl-1H-pyrazol-3-yl)methyl)-7-((2-(methylamino)-1H-imidazol-1-yl)methyl)-3,4-dihydroisoquinolin-1(2H)-one (87) was synthesized from 87-4 following the general procedure for coupling of cyclopropylboronic acid described in Example 78. ¹H NMR CD₃OD 400 MHz) δ=8.56 (s, 1H), 7.69 (s, 1H), 7.10 (s, 1H), 6.81 (d, J=2.0 Hz, 1H), 6.78 (d, J=2.1 Hz, 1H), 5.61 (s, 1H), 5.00 (s, 2H), 4.64 (s, 2H), 4.59 (br s, 2H), 4.12 (q, J=7.1 Hz, 2H), 3.64-3.56 (m, 5H), 3.15 (t, J=6.7 Hz, 2H), 2.95 (s, 3H), 2.00-1.90 (m, 1H), 1.40 (t, J=7.0 Hz, 3H), 1.04-0.96 (m, 2H), 0.66-0.59 (m, 2H). LCMS: [M+H]⁺=435.3.

### Example 88: Synthesis of (S)-2-(1-(4-ethoxy-5-fluoropyridin-2-yl)ethyl)-7-((2-(methylamino)-1H-imidazol-1-yl)methyl)-5-(pyrrolidin-1-yl)-3,4-dihydroisoquinolin-1(2H)-one (88)

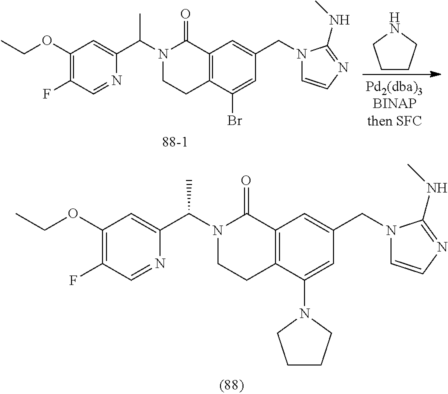

To a reaction tube were added compound 88-1 (20 mg, 0.040 mmol), Pd₂(dba)₃ (3.65 mg, 3.98 μmol), BINAP (7.44 mg, 0.012 mmol) and sodium tert-butoxide (11.48 mg, 0.119 mmol). Under N₂, pyrrolidine (8.49 mg, 0.119 mmol) and 1,4-dioxane (1 mL) were added, and the mixture was then stirred at 125° C. in a microwave reactor for 1 h. The reaction mixture was diluted with MeOH (5 mL) and purified by HPLC to give a racemic mixture. The racemic mixture was separated by chiral SFC (Column: (R,R)-WHELKO1, 5 um, 4.6 mm*250 mm, Condition: EtOH+0.5% NH₄OH) to give (S)-2-(1-(4-ethoxy-5-fluoropyridin-2-yl)ethyl)-7-((2-(methylamino)-1H-imidazol-1-yl)methyl)-5-(pyrrolidin-1-yl)-3,4-dihydroisoquinolin-1(2H)-one (88) (second peak on prep SFC). ¹H NMR (400 MHz, Methanol-d₄) δ ppm 8.23 (d, J=3.0 Hz, 1H), 7.37 (s, 1H), 7.13 (d, J=7.0 Hz, 1H), 6.86 (s, 1H), 6.60 (d, J=11.2 Hz, 2H), 5.94 (d, J=6.8 Hz, 1H), 4.91 (s, 2H), 4.20 (d, J=6.9 Hz, 2H), 3.46 (d, J=13.5 Hz, 2H), 3.15-3.02 (m, 4H), 2.86 (s, 4H), 2.79 (d, J=8.7 Hz, 1H), 1.92 (s, 4H), 1.61 (d, J=7.1 Hz, 3H), 1.43 (t, J=6.9 Hz, 3H). LCMS: [M+H]⁺=493.3.
Note: 5-bromo-2-(1-(4-ethoxy-5-fluoropyridin-2-yl)ethyl)-7-((2-(methylamino)-1H-imidazol-1-yl)methyl)-3,4-dihydroisoquinolin-1(2H)-one (88-1) was synthesized from intermediate (A1) and intermediate (G5) using general procedure for NaH mediated SN2 reaction described in Example 1. ¹H NMR (400 MHz, Methanol-d₄): δ 8.24 (d, J=3.3 Hz, 1H), 7.82 (s, 1H), 7.50 (d, J=1.5 Hz, 1H), 7.13 (d, J=6.8 Hz, 1H), 6.63 (dd, J=1.7, 18.6 Hz, 2H), 5.93 (q, J=7.1 Hz, 1H), 4.97 (s, 2H), 4.20 (dq, J=1.8, 7.0 Hz, 2H), 3.63-3.52 (m, 1H), 3.41 (ddd, J=5.4, 7.1, 12.7 Hz, 2H), 3.08-2.99 (m, 1H), 2.97-2.88 (m, 1H), 2.85 (s, 3H), 1.62 (d, J=7.1 Hz, 3H), 1.43 (t, J=7.0 Hz, 3H). LCMS: [M+H]⁺=502.1.

### Example 89: Synthesis of (S)-2-(1-(5-fluoro-4-methoxypyridin-2-yl)ethyl)-7-((2-(methylamino)-1H-imidazol-1-yl)methyl)-5-(pyrrolidin-1-yl)-3,4-dihydroisoquinolin-1(2H)-one (89)

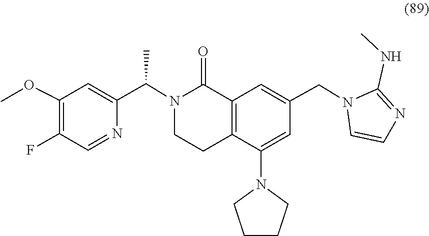 [SMILES: CNc1nccn1Cc1cc2c(c(N3CCCC3)c1)CCN([C@@H](C)c1cc(OC)c(F)cn1)C2=O]

(S)-2-(1-(5-fluoro-4-methoxypyridin-2-yl)ethyl)-7-((2-(methylamino)-1H-imidazol-1-yl)methyl)-5-(pyrrolidin-1-yl)-3,4-dihydroisoquinolin-1(2H)-one (89) was synthesized using the procedure described in Example 88, except compound (88-1) was replaced with compound (7-2). ¹H NMR (400 MHz, methanol-d₄) δ ppm 8.23 (d, J=3.2 Hz, 1H), 7.37 (s, 1H), 7.16 (d, J=6.9 Hz, 1H), 6.86 (s, 1H), 6.60 (dd, J=11.7, 1.7 Hz, 2H), 5.95 (q, J=7.1 Hz, 1H), 4.90 (s, 2H), 3.94 (s, 3H), 3.45 (ddd, J=13.3, 6.7, 3.2 Hz, 1H), 3.17-3.00 (m, 4H), 2.85 (s, 4H), 2.80 (dd, J=8.9, 5.2 Hz, 1H), 1.91 (t, J=6.3 Hz, 4H), 1.61 (d, J=7.1 Hz, 3H). LCMS: [M+H]⁺=479.3.

### Example 90: Synthesis of (S)-2-(1-(5-fluoro-4-methoxypyridin-2-yl)ethyl)-5-(3-methoxyazetidin-1-yl)-7-((2-(methylamino)-1H-imidazol-1-yl)methyl)-3,4-dihydroisoquinolin-1(2H)-one (90)

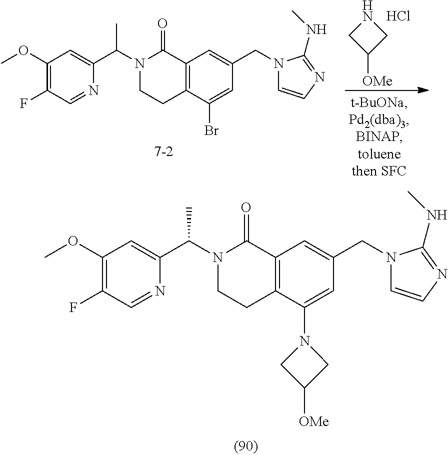

To a solution of compound 7-2 (30 mg, 0.06 mmol) in toluene (1 mL) was added 3-methoxyazetidine hydrochloride (23 mg, 0.18 mmol), t-BuONa (35 mg, 0.37 mmol) and BINAP (8 mg, 0.01 mmol). Pd₂(dba)₃ (6 mg, 0.006 mmol) was added to the mixture under N₂. The reaction mixture was stirred at 100° C. for 5 hrs. Then the mixture was concentrated and purified by Prep-HPLC (column: Phenomenex Synergi C18 150*25*10 um, Gradient: 15˜45% of B, A=water (0.225% FA)-ACN, B=ACN, FlowRate=25 ml/min) to give a racemic mixture which was then separated by chiral SFC (Column: (R,R)-WHELKO1, 5 um, 4.6 mm*250 mm, Condition: EtOH+0.5% NH₄OH) to give (S)-2-(1-(5-fluoro-4-methoxypyridin-2-yl)ethyl)-5-(3-methoxyazetidin-1-yl)-7-((2-(methylamino)-1H-imidazol-1-yl)methyl)-3,4-dihydroisoquinolin-1(2H)-one (90) (second peak on prep SFC). ¹H NMR (CD₃OD 400 MHz): δ 8.50 (br s, 1H), 8.23 (d, J=3.6 Hz, 1H), 7.29 (s, 1H), 7.16 (d, J=7.0 Hz, 1H), 6.93 (d, J=2.4 Hz, 1H), 6.88 (d, J=2.4 Hz, 1H), 6.63 (s, 1H), 5.99-5.88 (m, 1H), 5.02 (s, 2H), 4.34-4.23 (m, 1H), 4.13 (m, 2H), 3.95 (s, 3H), 3.67 (m, 2H), 3.53-3.42 (m, 1H), 3.38-3.32 (m, 1H), 3.31 (s, 4H), 2.99 (s, 3H), 2.85-2.67 (m, 2H), 1.62 (d, J=7.2 Hz, 3H). LCMS: MS (ESI) m/z 495.3 [M+H]⁺.

### Example 91: Synthesis of (S)-5-(difluoromethyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one (91)

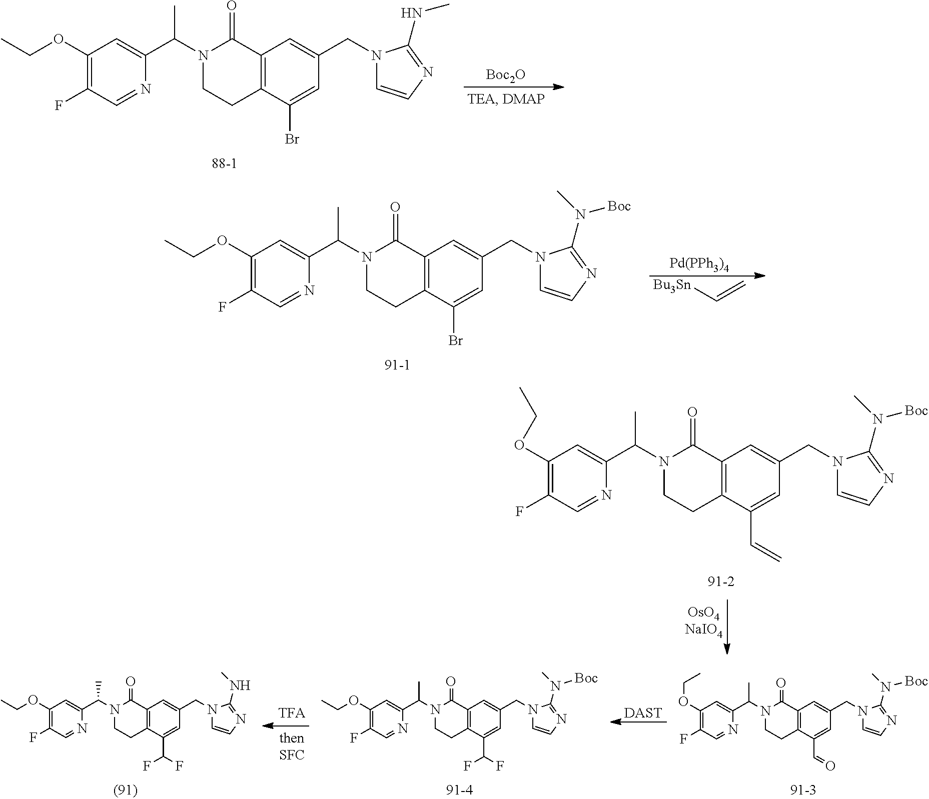

Step 1: To a solution of compound 88-1 (1.0 g, 2.00 mmol) and DMAP (24.3 mg, 0.2 mmol) in THF (15 mL) was added triethylamine (4.03 g, 40.00 mmol) and (Boc)₂O (6.5 g, 30.00 mmol). The mixture was stirred at 60° C. for 16 hrs. The mixture was added to water (40 mL) and extracted with ethyl acetate (50 mL×3). The combined organic layers were dried and concentrated, then purified by column chromatography (20-100% of EtOAc in hexane) to afford tert-butyl (1-((5-bromo-2-(1-(4-ethoxy-5-fluoropyridin-2-yl)ethyl)-1-oxo-1,2,3,4-tetrahydroisoquinolin-7-yl)methyl)-1H-imidazol-2-yl)(methyl)carbamate (91-1) (720 mg, 60% yield) as a pale yellow solid. ¹H NMR (400 MHz, Methanol-d₄) δ ppm 8.24 (d, J=3.2 Hz, 1H), 7.87 (s, 1H), 7.62 (d, J=1.5 Hz, 1H), 7.17-7.10 (m, 2H), 6.94 (d, J=1.3 Hz, 1H), 5.93 (q, J=7.1 Hz, 1H), 5.09 (br s, 2H), 4.26-4.15 (m, 2H), 3.65-3.53 (m, 1H), 3.43 (ddd, J=5.5, 7.2, 12.8 Hz, 1H), 3.11-2.99 (m, 2H), 2.98-2.89 (m, 3H), 2.01 (s, 1H), 1.62 (d, J=7.2 Hz, 4H), 1.56-1.27 (m, 13H). LC-MS: [M+H]⁺=602.4.

Step 2: A mixture of compound 91-1 (500 mg, 0.83 mmol), tributyl(vinyl)stannane (316 mg, 1.00 mmol) and Pd(PPh₃)₄ (96 mg, 0.083 mmol) in DMF (10 mL) was stirred under nitrogen atmosphere at 60° C. for 3 hrs. The reaction was cooled and KF (1 g) and water (30 mL) were added and the mixture stirred for 30 min. The resultant solution was extracted with ethyl acetate (40 mL×3). The combined organic layers were purified by column chromatography (20-100% of EtOAc in hexane) to afford tert-butyl (1-((2-(1-(4-ethoxy-5-fluoropyridin-2-yl)ethyl)-1-oxo-5-vinyl-1,2,3,4-tetrahydroisoquinolin-7-yl)methyl)-1H-imidazol-2-yl)(methyl)carbamate (91-2) (430 mg, 91% yield) as a yellow solid. LC-MS: [M+H]⁺=550.5

Step 3: OsO₄ (500 mg, 2 mmol) was dissolved in toluene (20 mL) to give a stock solution of OsO₄ in toluene (0.1 M). To a solution of compound 91-2 (430 mg, 0.782 mmol) in THF (12 mL) and water (4 mL) was added OsO₄ (1.17 mL, 0.117 mmol) and the mixture was then stirred at 25° C. for 15 min. To the light brown solution was added NaIO₄ (535.46 mg, 2.503 mmol). The mixture was stirred at 25° C. for 2 hrs. The reaction was filtered through Celite, washed with ethyl acetate (20 mL). The filtrated was added to water (20 mL) and extracted with ethyl acetate (20 mL×2). The combined organic layer was dried and concentrated, then purified by column chromatography (gradient: 50-100% of EtOAc in hexane) to afford tert-butyl (1-((2-(1-(4-ethoxy-5-fluoropyridin-2-yl)ethyl)-5-formyl-1-oxo-1,2,3,4-tetrahydroisoquinolin-7-yl)methyl)-1H-imidazol-2-yl)(methyl)carbamate (91-3) (200 mg, 40% yield) as a oil. LC-MS: [M+H]⁺=552.4

Step 4: To a solution of compound 91-3 (100 mg, 0.181 mmol) in dichloromethane (10 mL) was added DAST (789 mg, 4.895 mmol) at 0° C. The mixture was stirred at 0° C. for 2 hrs. The mixture was quenched with aq. NaHCO₃ (20 mL) and extracted with dichloromethane (20 mL×2). The combined organic layer were dried and concentrated to give tert-butyl (1-((5-(difluoromethyl)-2-(1-(4-ethoxy-5-fluoropyridin-2-yl)ethyl)-1-oxo-1,2,3,4-tetrahydroisoquinolin-7-yl)methyl)-1H-imidazol-2-yl)(methyl)carbamate (91-4) (140 mg, crude) as a light brown oil. LC-MS: [M+H]⁺=574.4.

Step 5: The solution of compound 91-4 (140 mg, crude) in TFA (3 mL) and dichloromethane (3 mL) was stirred at 25° C. for 3 hrs. The reaction was concentrated and purified by Prep-HPLC to give racemic compound (13.3 mg, 15.6%) as a yellow solid. Then it was separated by chiral SFC to afford (S)-5-(difluoromethyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one (91). ¹H NMR (400 MHz, Methanol-d₄) δ ppm 8.20 (d, J=3.2 Hz, 1H), 7.93 (d, J=1.9 Hz, 1H), 7.50-7.41 (m, 1H), 7.10 (d, J=6.8 Hz, 1H), 6.89 (t, J=54.9 Hz, 1H), 6.60 (dd, J=17.7, 1.7 Hz, 2H), 5.92 (q, J=7.1 Hz, 1H), 4.99 (s, 2H), 4.16 (qd, J=7.0, 2.4 Hz, 2H), 3.46 (dddd, J=62.4, 12.6, 8.1, 5.0 Hz, 2H), 3.10-2.89 (m, 2H), 2.81 (s, 3H), 1.59 (d, J=7.1 Hz, 3H), 1.40 (t, J=7.0 Hz, 3H). LC-MS: [M+H]⁺=474.1.

### Example 92: Synthesis of (S)-5-(difluoromethyl)-2-(1-(5-fluoro-4-methoxypyridin-2-yl)ethyl)-7-((2-(methylamino)-1H-imidazol-1-yl)methyl)-3,4-dihydroisoquinolin-1(2H)-one (92)

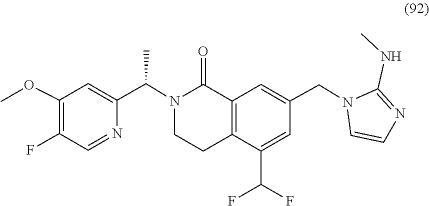 [SMILES: CNc1nccn1Cc1cc2c(c(C(F)F)c1)CCN([C@@H](C)c1cc(OC)c(F)cn1)C2=O]

(S)-5-(difluoromethyl)-2-(1-(5-fluoro-4-methoxypyridin-2-yl)ethyl)-7-((2-(methylamino)-1H-imidazol-1-yl)methyl)-3,4-dihydroisoquinolin-1(2H)-one (92) was synthesized following a similar method as described in Example 91, except compound 88-1 was replaced with compound 7-2. ¹H NMR (400 MHz, Methanol-d₄) δ ppm 8.24 (d, J=3.2 Hz, 1H), 7.96 (s, 1H), 7.49 (s, 1H), 7.17 (d, J=6.9 Hz, 1H), 6.93 (t, J=54.9 Hz, 1H), 6.65 (d, J=1.7 Hz, 1H), 6.61 (d, J=1.7 Hz, 1H), 5.97 (q, J=7.1 Hz, 1H), 5.03 (s, 2H), 3.95 (s, 3H), 3.58 (ddd, J=13.7, 9.1, 4.8 Hz, 1H), 3.43 (ddd, J=12.6, 7.1, 5.2 Hz, 1H), 3.12-2.92 (m, 2H), 2.84 (s, 3H), 1.63 (d, J=7.1 Hz, 3H). LC-MS: [M+H]⁺=460.0.

### Example 93: Synthesis of (S)-5-(difluoromethyl)-2-(1-(6-fluoro-5-methoxypyridin-3-yl)ethyl)-7-((2-(methylamino)-1H-imidazol-1-yl)methyl)-3,4-dihydroisoquinolin-1(2H)-one (93)

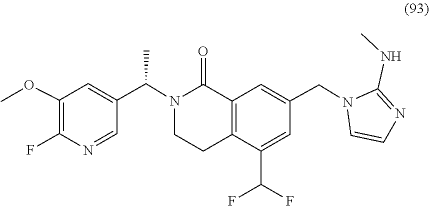 [SMILES: CNc1nccn1Cc1cc2c(c(C(F)F)c1)CCN([C@@H](C)c1cnc(F)c(OC)c1)C2=O]

(S)-5-(difluoromethyl)-2-(1-(6-fluoro-5-methoxypyridin-3-yl)ethyl)-7-((2-(methylamino)-1H-imidazol-1-yl)methyl)-3,4-dihydroisoquinolin-1(2H)-one (93) was synthesized following the same procedure as in Example 91, except compound 88-1 was replaced with compound 78-1. ¹H NMR (400 MHz, Methanol-d₄) δ ppm 7.96 (s, 1H), 7.71 (d, J=2.0 Hz, 1H), 7.53-7.42 (m, 2H), 6.90 (t, J=54.9 Hz, 1H), 6.61 (dd, J=18.4, 1.8 Hz, 2H), 6.04 (q, J=7.1 Hz, 1H), 5.01 (s, 2H), 3.87 (s, 3H), 3.53 (ddd, J=13.3, 9.2, 4.7 Hz, 1H), 3.26-3.21 (m, 1H), 3.14-2.90 (m, 2H), 2.82 (s, 3H), 1.63 (d, J=7.1 Hz, 3H). LC-MS: [M+H]⁺=460.2.

### Example 94: Synthesis of (S)-2-(1-(6-fluoro-5-methoxypyridin-3-yl)ethyl)-7-((2-(methylamino)-1H-imidazol-1-yl)methyl)-3,4-dihydroisoquinolin-1(2H)-one (94)

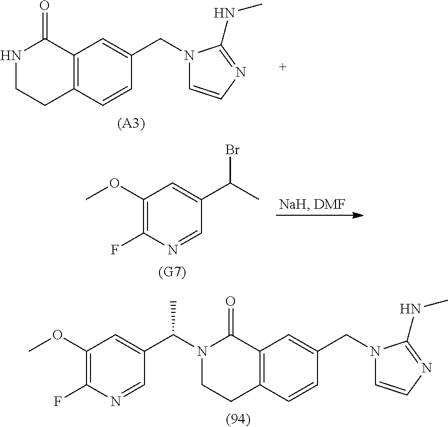

To a solution of intermediate (A3) (10.2 g, 40 mmol) in DMF (120 mL) was added NaH (2 g, 50 mmol, 60% content) under nitrogen atmosphere at 0° C. and the mixture was stirred at 0° C. for 15 mins. A solution of intermediate (G7) (11.2 g, 47.7 mmol) in DMF (30 mL) was then added dropwise at 0° C. The solution was stirred at 0° C. for 1 hr. The reaction was quenched with water (600 mL) at 0° C. and diluted with brine (200 mL). The mixture was extracted with DCM (300 mL×3). The combined organic layers were dried and concentrated, then purified by flash column chromatography (0˜3% of methanol in EtOAc with 0.4% NH₃H₂O) to afford product as a yellow solid, which was further separated by chiral SFC (Column: IC (250 mm*30 mm, 10 um), Condition: 0.1% NH₃H₂O MEOH) to give (S)-2-(1-(6-fluoro-5-methoxypyridin-3-yl)ethyl)-7-((2-(methylamino)-1H-imidazol-1-yl)methyl)-3,4-dihydroisoquinolin-1(2H)-one (94) (second peak on SFC, 3.6 g). ¹H-NMR (400 MHz, Methanol-d₄) δ ppm 7.83 (s, 1H), 7.70 (s, 1H), 7.45 (m, 1H), 7.27-7.17 (m, 2H), 6.59 (m, 2H), 6.06 (m, 1H), 4.95 (s, 2H), 3.87 (s, 3H), 3.57-3.44 (m, 1H), 3.20 (m, 1H), 2.95-2.77 (m, 5H), 1.62 (d, J=7.3 Hz, 3H). LC-MS: [M+H]⁺=410.1.

### Example 95: Synthesis of (S)-2-(1-(6-chloro-5-ethoxypyridin-3-yl)ethyl)-7-((2-(methylamino)-1H-imidazol-1-yl)methyl)-3,4-dihydroisoquinolin-1(2H)-one (95)

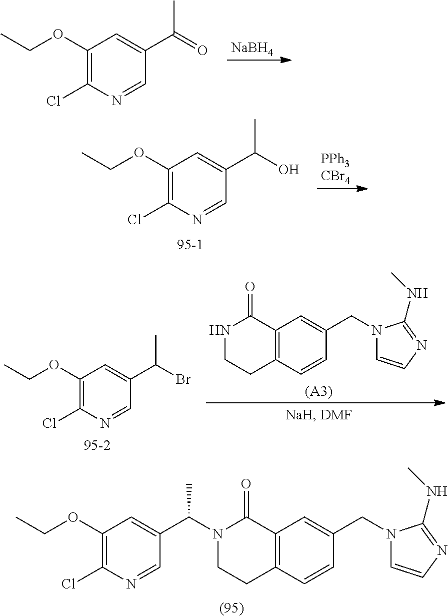

Step 1: To the reaction tube was added 1-(6-chloro-5-ethoxypyridin-3-yl)ethan-1-one (95 mg, 0.476 mmol), CH₂Cl₂ (2 mL) and EtOH (2.0 mL), then at 0° C., NaBH₄ (36.0 mg, 0.952 mmol) was added, warm to rt, stirred for 1 h. The reaction was quenched with NH4Cl (aq.), extracted with EtOAc, dried and concentrated, then purified by flash column (50% EtOAc in Hexane) to give product 95-1 (80 mg, 83%). LC-MS: [M+H]⁺=202.1.

Step 2: To the tube was added 95-1 (80 mg, 0.397 mmol), CH₂Cl₂ (6 mL), at 0° C., triphenylphosphine (156 mg, 0.595 mmol) and perbromomethane (197 mg, 0.595 mmol) in DCM (1 mL) was added, then stirred at rt for 3 h. Then solvent was removed, the residue was purified by flash column (20% EtOAc in Hexane) to give compound 95-2 (79 mg, 75%). LC-MS: [M+H]⁺=266.0.

Step 3: (S)-2-(1-(6-chloro-5-ethoxypyridin-3-yl)ethyl)-7-((2-(methylamino)-1H-imidazol-1-yl)methyl)-3,4-dihydroisoquinolin-1(2H)-one (95) was synthesized following the same procedure described in Example 94, except intermediate (G7) was replaced with 95-2. ¹H NMR (400 MHz, Methanol-d₄) δ ppm 7.96 (s, 1H), 7.84 (s, 1H), 7.40 (s, 1H), 7.24 (t, J=5.8 Hz, 2H), 6.60 (dd, J=13.3, 1.5 Hz, 2H), 6.08 (q, J=7.1 Hz, 1H), 4.97 (s, 2H), 4.24-4.03 (m, 2H), 3.54 (ddd, J=12.8, 9.1, 5.0 Hz, 1H), 3.23 (dt, J=12.3, 6.1 Hz, 1H), 2.97-2.87 (m, 1H), 2.84 (s, 3H), 1.65 (d, J=7.1 Hz, 3H), 1.43 (t, J=7.0 Hz, 3H). LC-MS: [M+H]⁺=440.2.

### Example 96: Synthesis of 3-ethoxy-5-((7-((2-(methylamino)-1H-imidazol-1-yl)methyl)-1-oxo-3,4-dihydroisoquinolin-2(1H)-yl)methyl)picolinonitrile (96)

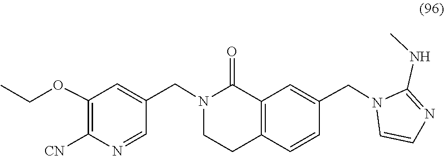

3-ethoxy-5-((7-((2-(methylamino)-1H-imidazol-1-yl)methyl)-1-oxo-3,4-dihydroisoquinolin-2(1H)-yl)methyl)picolinonitrile (96) was synthesized following the same procedure described in Example 94, except intermediate (G7) was replaced with intermediate (G4). ¹H NMR (400 MHz, Methanol-d₄) δ ppm 8.23 (d, J=1.5 Hz, 1H), 7.82 (d, J=1.4 Hz, 1H), 7.62 (d, J=1.3 Hz, 1H), 7.44-7.25 (m, 2H), 6.91-6.75 (m, 2H), 5.07 (s, 2H), 4.85 (s, 2H), 4.24 (q, J=7.0 Hz, 2H), 3.66 (t, J=6.7 Hz, 2H), 3.05 (t, J=6.7 Hz, 2H), 2.95 (s, 3H), 1.46 (t, J=7.0 Hz, 3H). LC-MS: [M+H]⁺=417.3.

### Example 97: Synthesis of (S)-3-ethoxy-5-(1-(7-((2-(methylamino)-1H-imidazol-1-yl)methyl)-1-oxo-3,4-dihydroisoquinolin-2(1H)-yl)ethyl)picolinonitrile (97)

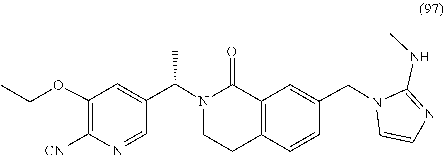

(S)-3-ethoxy-5-(1-(7-((2-(methylamino)-1H-imidazol-1-yl)methyl)-1-oxo-3,4-dihydroisoquinolin-2(1H)-yl)ethyl)picolinonitrile (97) was synthesized following the same procedure described in Example 94, except intermediate (G7) was replaced with intermediate (G3) 5-(1-bromoethyl)-3-ethoxy-2-isocyanopyridine. ¹H NMR (400 MHz, Methanol-d₄) δ ppm 8.27 (d, J=1.3 Hz, 1H), 7.82 (s, 1H), 7.60-7.52 (m, 1H), 7.32-7.17 (m, 2H), 6.59 (dd, J=13.6, 1.7 Hz, 2H), 6.08 (q, J=7.1 Hz, 1H), 4.96 (s, 2H), 4.26 (qd, J=7.0, 2.7 Hz, 2H), 3.57 (ddd, J=12.7, 8.9, 5.3 Hz, 1H), 3.34-3.24 (m, 3H), 2.96-2.88 (m, 2H), 1.68 (d, J=7.2 Hz, 3H), 1.46 (t, J=7.0 Hz, 3H). LC-MS: [M+H]⁺=431.3.

### Example 98: 5-(2-chloro-5-(hydroxymethyl)pyridin-3-yl)-2-(3-(difluoromethoxy)benzyl)-7-((2-(methylamino)-1H-imidazol-1-yl)methyl)isoquinolin-1(2H)-one (98)

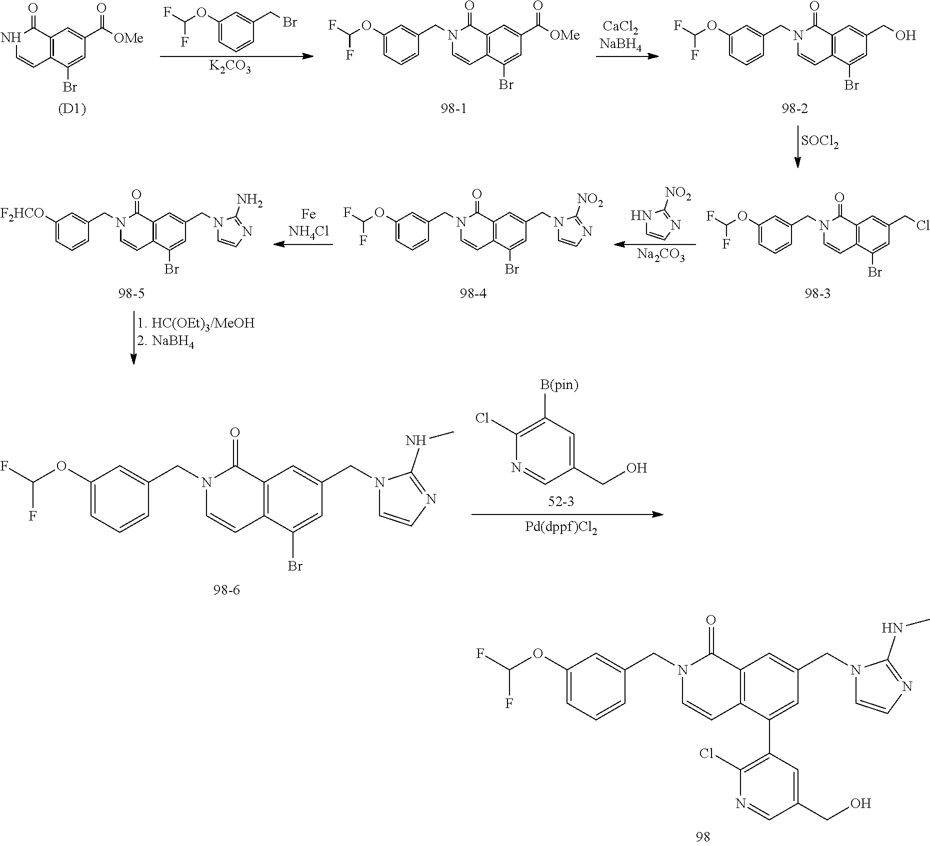

Step 1: To a mixture of intermediate (D1) (400 mg, 1.42 mmol) and K₂CO₃ (392 mg, 2.84 mmol) in DMF (10 mL) was added 1-(bromomethyl)-3-(difluoromethoxy)benzene (370 mg, 1.56 mmol). The mixture was stirred at 20° C. for 20 hours. The mixture was poured into water (100 mL). The resulting mixture was extracted with ethyl acetate (20 mL×2). The combined organic layers were dried and concentrated, then purified by flash column (10-30% of ethyl acetate in Hexane) to afford methyl 5-bromo-2-(3-(difluoromethoxy)benzyl)-1-oxo-1,2-dihydroisoquinoline-7-carboxylate (98-1) (530 mg, 85% yield) as a yellow jelly. ¹H-NMR (400 MHz, Methanol-d₄) δ ppm 8.95 (d, J=0.8 Hz, 1H), 8.49 (d, J=2.0 Hz, 1H), 7.71 (d, J=7.6 Hz, 1H), 7.41-7.35 (m, 1H), 7.21 (d, J=8.0 Hz, 1H), 7.17 (s, 1H), 7.09 (d, J=8.0 Hz, 1H), 7.00-6.60 (m, 2H), 5.27 (s, 2H), 3.97 (s, 3H).

Step 2: To a mixture of CaCl₂ (190 mg, 1.71 mmol) in tetrahydrofuran/ethanol (1/1, 100 mL) was added NaBH₄ (130 mg, 3.42 mmol) at 0° C. under nitrogen atmosphere. After the mixture was stirred at 0° C. for 10 min, compound 98-1 (500 mg, 1.14 mmol) was added to the mixture at 0° C. and the mixture was stirred at 0° C. for 3 hrs. Water (50 mL) was added and the mixture was extracted with ethyl acetate (30 mL×3). The combined organic layers were dried and concentrated to afford 5-bromo-2-(3-(difluoromethoxy)benzyl)-7-(hydroxymethyl)isoquinolin-1(2H)-one (98-2) (500 mg, 100% yield) as a yellow solid. LC-MS: [M+H]⁺=409.9.

Step 3: To a solution of compound 98-2 (500 mg, 1.14 mmol) in dichloromethane (30 mL) was added thionyl chloride (832 uL, 1.56 g/mL, 11.4 mmol). The mixture was stirred at 0° C. for 2 hrs. The mixture was adjusted to pH 7-8 with sat.NaHCO₃. The organic layer was dried and concentrated, then purified by flash column (10%-30% of ethyl acetate in hexane to EtOAc/CH₂Cl₂=1/1) to afford 5-bromo-7-(chloromethyl)-2-(3-(difluoromethoxy)benzyl)isoquinolin-1(2H)-one (98-3) (430 mg, 88% yield) as a light yellow solid. LC-MS: [M+H]⁺=429.7.

Step 4: To a mixture of compound 98-3 (430 mg, 1.0 mmol) and 2-nitro-1H-imidazole (124 mg, 1.1 mmol) in DMF (10 mL) was added Na₂CO₃ (212 mg, 2.0 mmol). The mixture was stirred at 80° C. for 18 hrs. The mixture was poured into water (150 mL). The resulting mixture was extracted with ethyl acetate (20 mL×3). The combined organic layers were washed with brine (30 mL×3), dried and concentrated to afford 5-bromo-2-(3-(difluoromethoxy)benzyl)-7-((2-nitro-1H-imidazol-1-yl)methyl)isoquinolin-1(2H)-one (98-4) (460 mg, 91% yield) as a yellow jelly. LC-MS: [M+H]⁺=529.

Step 5: To a mixture of compound 98-4 (460 mg, 0.91 mmol) and NH₄Cl (49 mg, 0.91 mmol) in ethanol/H₂O (15 mL/5 mL) was added Fe (153 mg, 2.73 mmol). The mixture was stirred at 80° C. for 2 h. The mixture was filtered. The filtrate was concentrated to afford 7-((2-amino-1H-imidazol-1-yl)methyl)-5-bromo-2-(3-(difluoromethoxy)benzyl)isoquinolin-1(2H)-one (98-5) (460 mg, 100% yield) as a yellow solid. LC-MS: [M+H]⁺=476.8.

Step 6: A mixture of compound 98-5 (230 mg, 0.48 mmol) in triethyl orthoformate (10 mL) and methanol (1 mL) was stirred at 140° C. for 20 h. The mixture was concentrated, giving a a reddish brown jelly, to which ethanol (40 mL) was added Then NaBH₄ (53 mg, 1.41 mmol) was added at 0° C. under nitrogen atmosphere, and the mixture was stirred at 4-9° C. for 16 hrs. The mixture was quenched with water (5 mL), and then concentrated and purified by flash column (0-5% of methanol in dichloromethane) to afford 5-bromo-2-(3-(difluoromethoxy)benzyl)-7-((2-(methylamino)-1H-imidazol-1-yl)methyl)isoquinolin-1(2H)-one (98-6) (120 mg, 52% yield) as a yellow solid. ¹H-NMR (400 MHz, Methanol-d₄) δ ppm 8.13 (s, 1H), 7.77 (d, J=2.0 Hz, 1H), 7.57 (d, J=7.6 Hz, 1H), 7.37 (t, J=8.0 Hz, 1H), 7.18 (d, J=8.0 Hz, 1H), 7.13 (s, 1H), 7.08 (d, J=8.0 Hz, 1H), 6.93 (d, J=7.6 Hz, 1H), 6.80 (t, J=74.0 Hz, 1H), 6.72 (d, J=1.6 Hz, 1H), 6.66 (d, J=1.6 Hz, 1H), 5.24 (s, 2H), 5.09 (s, 2H), 2.87 (s, 3H). LC-MS: [M+H]⁺=491.0.

Step 7: To a mixture of compound 98-6 (120 mg, 0.245 mmol), compound 52-3 (132 mg, 0.49 mmol) and Na₂CO₃ (53 mg, 0.49 mmol) in dioxane/H₂O (10 mL/1 mL) was added Pd(dppf)Cl₂ (18 mg, 0.02 mmol) under nitrogen atmosphere. The mixture was stirred at 90° C. for 2 hrs. The mixture was concentrated and purified by preparative HPLC (column: Phenomenex Gemini 150*25 mm*10 um, gradient: 20-40 of B, A=0.05 HCl water, B=acetonitrile, flow rate: 25 mL/min) to afford 5-(2-chloro-5-(hydroxymethyl)pyridin-3-yl)-2-(3-(difluoromethoxy)benzyl)-7-((2-(methylamino)-1H-imidazol-1-yl)methyl)isoquinolin-1(2H)-one (98) as a pale yellow solid. ¹H-NMR (400 MHz, Methanol-d₄) δ ppm 8.52 (d, J=2.0 Hz, 1H), 8.26 (s, 1H), 7.88 (d, J=2.0 Hz, 1H), 7.63 (s, 1H), 7.51 (d, J=7.6 Hz, 1H), 7.38 (t, J=8.0 Hz, 1H), 7.21 (d, J=7.6 Hz, 1H), 7.14 (s, 1H), 7.08 (d, J=8.0 Hz, 1H), 7.04 (d, J=2.4 Hz, 2H), 6.81 (t, J=74.0 Hz, 1H), 6.25 (d, J=7.6 Hz, 1H), 5.36-5.20 (s, 4H), 4.75 (s, 2H), 3.03 (s, 3H). LC-MS: [M+H]⁺=552.1.

Following a similar procedure to that of Example 98 (unless otherwise specified). The following compounds were prepared from their corresponding precursors.

| Ex. | Name/Structure | ¹HNMR/LC-MS | Precursors |
|---|---|---|---|
| 99 | 5-bromo-2-(4-fluoro-3- | ¹H NMR (CD3OD 400 MHz) δ 8.10 | Step 1: 1- |
| | methoxybenzyl)-7-((2- | (s, 1H), 7.94 (d, J = 1.6 Hz, 1H), | (bromomethyl)-3- |
| | (methylamino)-1H-imidazol-1- | 7.64 (d, J = 7.6 Hz, 1H), 7.14 (dd, | (difluoromethoxy) |
| | yl)methyl)isoquinolin-1(2H)-one | J = 8.0, 2.0 Hz, 1H), 7.07-6.99 (m, | benzene was |
| | 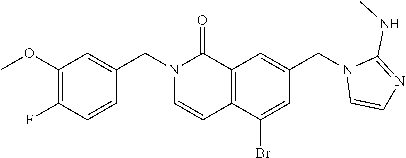 [SMILES: CNc1nccn1Cc1cc(Br)c2ccn(Cc3ccc(F)c(OC)c3)c(=O)c2c1] | 3H), 6.96 (d, J = 7.6 Hz, 1H), 6.92- 6.85 (m, 1H), 5.20 (d, J = 2.4 Hz, 4H), 3.85 (s, 3H), 3.00 (s, 3H). LC-MS″ (ESI) m/z 473.0 [M + H]⁺ | replaced with 4- (bromomethyl)-1- fluoro-2- methoxybenzene Step 7 not required |
| 100 | 5-(2-chloro-5- | ¹H NMR (400 MHz, CD₃OD) δ 8.47 | Step 1: 1- |
| | (hydroxymethyl)pyridin-3-yl)-2-(3- | (d, J = 13 Hz, 2H), 8.25 (s, 1H), | (bromomethyl)-3- |
| | methoxybenzyl)-7-((2- | 7.81 (s, 1H), 7.55 (s, 1H), 7.42 (d, | (difluoromethoxy) |
| | (methylamino)-1H-imidazol-1- | J = 6 Hz, 1H), 7.22 (t, J = 6 Hz, 1H), | benzene was |
| | yl)methyl)isoquinolin-1(2H)-one | 6.96-6.83 (m, 5H), 6.20 (d, J = 2 | replaced with 1- |
| | 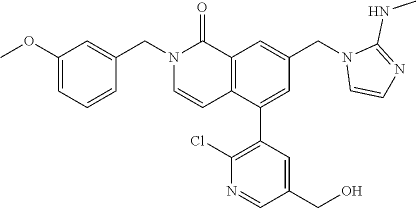 | Hz, 1H), 5.32-5.12 (m, 4H), 4.71 (s, 2H), 3.75 (s, 3H), 3.33 (s, 3H). LC-MS: (ESI) m/z 516.2 [M + H]⁺ | (bromomethyl)-3- methoxybenzene Step 7: compound 52-3 |
| 101 | 5-(2-chloro-5- | ¹H NMR (CD3OD 400 MHz) δ 8.46 | Step 1: 1- |
| | (hydroxymethyl)pyridin-3-yl)-2-(4- | (d, J = 2.4 Hz, 1H), 8.28 (d, J = 1.2 | (bromomethyl)-3- |
| | fluoro-3-methoxybenzyl)-7-((2- | Hz, 1H), 7.78 (d, J = 2.4 Hz, 1H), | (difluoromethoxy) |
| | (methylamino)-1H-imidazol-1- | 7.45-7.38 (m, 2H), 7.13 (dd, J = 8.4, | benzene was |
| | yl)methyl)isoquinolin-1(2H)-one | 2.0 Hz, 1H), 7.06-7.01 (m, 1H), | replaced with 4- |
| | 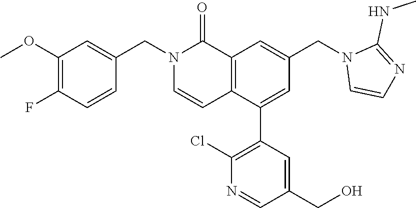 | 6.91-6.84 (m, 1H), 6.70 (d, J = 1.6 Hz, 1H), 6.62 (d, J = 1.6 Hz, 1H), 6.18 (d, J = 8.0 Hz, 1H), 5.26-5.13 (m, 4H), 4.70 (s, 2H), 3.84 (s, 3H), 2.86 (s, 3H). LC-MS: (ESI) m/z 534.1 [M + H]⁺ | (bromomethyl)-1- fluoro-2- methoxybenzene Step 7: compound 52-3 |
| 102 | (S)-5-(2-chloro-5- | ¹H NMR (CD3OD 400 MHz) δ 8.47 | Step 1: 1- |
| | (hydroxymethyl)pyridin-3-yl)-2-(1- | (d, J = 2.0 Hz, 1H), 8.28 (s, 1H), | (bromomethyl)-3- |
| | (3-methoxyphenyl)ethyl)-7-((2- | 7.80 (dd, J = 8.4, 2.4 Hz, 1H), 7.57 | (difluoromethoxy) |
| | (methylamino)-1H-imidazol-1- | (d, J = 1.6 Hz, 1H), 7.33-7.22 (m, | benzene was |
| | yl)methyl)isoquinolin-1(2H)-one | 2H), 7.03 (s, 2H), 6.97-6.82 (m, | replaced with 1-(1- |
| | 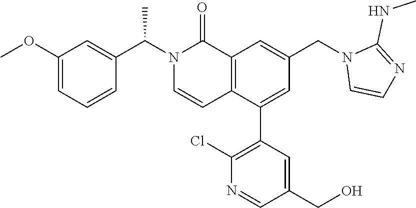 | 3H), 6.38 (q, J = 7.6 Hz, 1H), 6.20 (dd, J = 8.4, 2.8 Hz, 1H), 5.29 (d, J = 2.4 Hz, 2H), 4.71 (d, J = 8.0 Hz, 2H), 3.76 (d, J = 2.8 Hz, 3H), 3.02 (s, 3H), 1.78 (t, J = 7.2 Hz, 3H). LC- MS: (ESI) m/z 530.1 [M + H]⁺ | bromoethyl)-3- methoxybenzene Step 7: compound 52-3 (followed by SFC separation) |
| 103 | (R)-5-(2-chloro-5- | ¹H NMR (CD3OD 400 MHz) δ 8.48 | Step 1: 1- |
| | (hydroxymethyl)pyridin-3-yl)-2-(1- | (d, J = 1.6 Hz, 1H), 8.29 (s, 1H), | (bromomethyl)-3- |
| | (3-methoxyphenyl)ethyl)-7-((2- | 7.80 (dd, J = 8.4, 2.4 Hz, 1H), 7.58 | (difluoromethoxy) |
| | (methylamino)-1H-imidazol-1- | (d, J = 1.6 Hz, 1H), 7.33-7.28 (m, | benzene was |
| | yl)methyl)isoquinolin-1(2H)-one | 2H), 7.03 (s, 2H), 6.94-6.88 (m, | replaced with 1-(1- |
| | 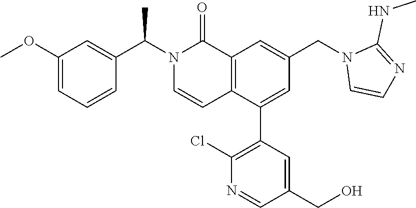 | 3H), 6.40-6.22 (m, 1H), 6.20 (dd, J = 8.4, 2.8 Hz, 1H), 5.29 (d, J = 2.8 Hz, 2H), 4.72 (d, J = 8.4 Hz, 2H), 3.76 (d, J = 2.8 Hz, 3H), 3.04 (s, 3H), 1.78 (t, J = 8.0 Hz, 3H). LC- MS: (ESI) m/z 530.1 [M + H]⁺ | bromoethyl)-3- methoxybenzene Step 7: compound 52-3 (followed by SFC separation) |
| 104 | 5-(2-chloro-5- | ¹H NMR (CD3OD 400 MHz) δ 8.48 | Step 1: 1- |
| | (hydroxymethyl)pyridin-3-yl)-2-(3- | (d, J = 2.0 Hz, 1H), 8.24 (s, 1H), | (bromomethyl)-3- |
| | fluoro-4-methoxybenzyl)-7-((2- | 7.82 (s, 1H), 7.58 (s, 1H), 7.47 (d, | (difluoromethoxy) |
| | (methylamino)-1H-imidazol-1- | J = 7.6 Hz, 1H), 7.13 (d, J = 10.4 Hz, | benzene was |
| | yl)methyl)isoquinolin-1(2H)-one | 2H), 7.06 (d, J = 8.4 Hz, 1H), 7.02 | replaced with 4- |
| | 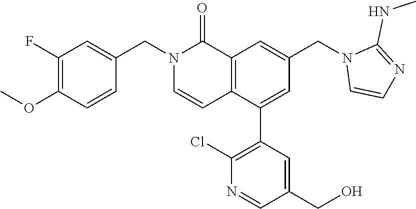 | (s, 2H), 6.21 (d, J = 7.6 Hz, 1H), 5.29 (s, 2H), 5.24-5.09 (m, 2H), 4.72 (s, 2H), 3.84 (s, 3H), 3.02 (s, 3H). LC-MS: (ESI) m/z 534.1 [M + H]⁺ | (bromomethyl)-2- fluoro-1- methoxybenzene Step 7: compound 52-3 |
| 105 | 5-(2-chloro-5- | ¹H NMR (DMSO-d6 400 MHz) δ | Step 1: 1- |
| | (hydroxymethyl)pyridin-3-yl)-2-(4- | 12.51 (br. s, 1H), 8.48 (d, J = 2.4 | (bromomethyl)-3- |
| | methoxybenzyl)-7-((2- | Hz, 1H), 8.27 (br. s, 1H), 8.20 (s, | (difluoromethoxy) |
| | (methylamino)-1H-imidazol-1- | 1H), 7.75 (d, J = 2.0 Hz, 1H), 7.64 | benzene was |
| | yl)methyl)isoquinolin-1(2H)-one | (d, J = 1.6 Hz, 1H), 7.59 (d, J = 7.6 | replaced with 1- |
| | 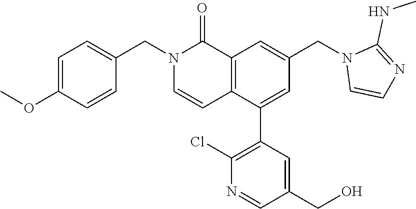 | Hz, 1H), 7.29 (d, J = 8.8 Hz, 2H), 7.21 (s, 1H), 7.13 (s, 1H), 6.88 (d, J = 8.8 Hz, 2H), 6.01 (d, J = 8.0 Hz, 1H), 5.27 (d, J = 4.8 Hz, 2H), 5.17- 5.02 (m, 2H), 4.60 (s, 2H), 3.70 (s, 3H), 2.90 (d, J = 4.8 Hz, 3H). LC- MS: (ESI) m/z 516.1 [M + H]⁺ | (bromomethyl)-4- methoxybenzene Step 7: compound 52-3 |
| 106 | 5-(2-chloro-5-(1- | ¹H NMR (400 MHz, Methanol-d4) δ | Step 1: 1- |
| | hydroxyethyl)pyridin-3-yl)-2-(4- | 8.49 (q, J = 5.9, 4.5 Hz, 1H), 8.23 | (bromomethyl)-3- |
| | fluoro-3-methoxybenzyl)-7-((2- | (s, 1H), 7.82 (t, J = 3.3 Hz, 1H), | (difluoromethoxy) |
| | (methylamino)-1H-imidazol-1- | 7.57 (d, J = 5.5 Hz, 1H), 7.49 (dd, | benzene was |
| | yl)methyl)isoquinolin-1(2H)-one | J = 7.9, 2.3 Hz, 1H), 7.14 (d, J = 8.1 | replaced with 4- |
| | 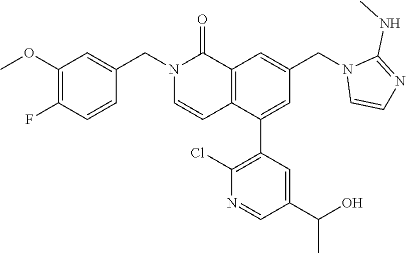 [SMILES: CNc1nccn1Cc1cc(-c2cc(C(C)O)cnc2Cl)c2ccn(Cc3ccc(F)c(OC)c3)c(=O)c2c1] | Hz, 1H), 7.02 (d, J = 7.8 Hz, 3H), 6.87 (d, J = 8.5 Hz, 1H), 6.20 (d, J = 7.5 Hz, 1H), 5.27 (d, J = 3.3 Hz, 2H), 5.24-5.11 (m, 2H), 4.97 (d, J = 6.8 Hz, 1H), 3.84 (s, 3H), 3.01 (s, 3H), 1.50 (d, J = 6.5 Hz, 3H). LC-MS: (ESI) m/z 548.3 [M + H]⁺ | (bromomethyl)-1- fluoro-2- methoxybenzene Step 7: compound 76-2 |

### Example 107: Synthesis of 5-(2-chloro-5-(hydroxymethyl)pyridin-3-yl)-2-((4-methyl-1H-indol-2-yl)methyl)-7-((2-(methylamino)-1H-imidazol-1-yl)methyl)isoquinolin-1(2H)-one (107)

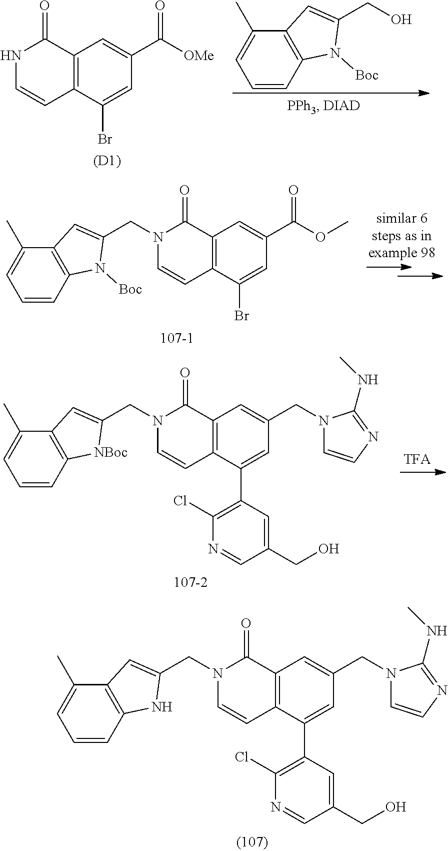

Step 1: To a mixture of intermediate (D1) (1.25 g, 4.4 mmol), tert-butyl 2-(hydroxymethyl)-4-methyl-1H-indole-1-carboxylate (1.15 g, 4.4 mmol) and PPh₃ (3.46 g, 13.2 mmol) in THF (100 mL) was added DIAD (2.67 g, 13.2 mmol) dropwise at 0° C. under the nitrogen atmosphere. The mixture solution was stirred at 50° C. for 18 hours under nitrogen atmosphere. The reaction was quenched with ice water (15 mL) and extracted with ethyl acetate (20 mL×2). The combined organic layers were washed with brine (10 mL), then dried, concentrated and purified on preparative HPLC (Boston Green ODS 150*30 5 u, gradient: 88-98% B (A=0.1% TFA water, B=acetonitrile), flow rate: 30 mL/min) to afford methyl 5-bromo-2-((1-(tert-butoxycarbonyl)-4-methyl-1H-indol-2-yl)methyl)-1-oxo-1,2-dihydroisoquinoline-7-carboxylate (107-1) (200 mg, yield: 6.1%) as a white solid. ¹H NMR (d6-DMSO 400 MHz): δ 8.81 (s, 1H), 8.44 (s, 1H), 7.90 (d, J=8.4 Hz, 1H), 7.80 (d, J=7.6 Hz, 1H), 7.19 (t, J=8.0 Hz, 1H), 7.00 (d, J=7.2 Hz, 1H), 6.88 (d, J=7.6 Hz, 1H), 6.24 (s, 1H), 5.53 (s, 2H), 3.92 (s, 3H), 2.32 (s, 3H), 1.63 (s, 9H).

Steps 2-7: tert-butyl 2-((5-(2-chloro-5-(hydroxymethyl)pyridin-3-yl)-7-((2-(methylamino)-1H-imidazol-1-yl)methyl)-1-oxoisoquinolin-2(1H)-yl)methyl)-4-methyl-1H-indole-1-carboxylate (107-2) was synthesized following a similar procedure to steps 2-7 described in Example 98, except compound 98-1 was replaced with compound 107-1. LCMS: [M+H]⁺=638.9.

Step 8: To the solution of compound 107-2 (22 mg, 0.034 mmol) in DCM (2 mL) was added TFA (2.001 mL, 26.0 mmol) and the solution stirred at 25° C. for 2 hrs. The mixture was concentrated and purified by preparative-HPLC to give 5-(2-chloro-5-(hydroxymethyl)pyridin-3-yl)-2-((4-methyl-1H-indol-2-yl)methyl)-7-((2-(methylamino)-1H-imidazol-1-yl)methyl)isoquinolin-1(2H)-one (107) (2.3 mg, 11.8 yield) as white solid. ¹H NMR (400 MHz, Methanol-d4) δ 8.46 (d, J=2.3 Hz, 1H), 8.34 (s, 1H), 7.77 (d, J=2.3 Hz, 1H), 7.48-7.36 (m, 2H), 7.15 (d, J=8.2 Hz, 1H), 6.98 (t, J=7.7 Hz, 1H), 6.79 (d, J=7.1 Hz, 1H), 6.71 (d, J=1.7 Hz, 1H), 6.63 (d, J=1.6 Hz, 1H), 6.49 (s, 1H), 6.18 (d, J=7.6 Hz, 1H), 5.45 (d, J=15.0 Hz, 1H), 5.32 (d, J=14.9 Hz, 1H), 5.17 (s, 2H), 4.70 (s, 2H), 2.88 (s, 3H), 2.47 (s, 3H). LCMS: [M+H]⁺=539.0.

### Example 108: Synthesis of 2-((1H-indol-2-yl)methyl)-5-(2-chloro-5-(hydroxymethyl)pyridin-3-yl)-7-((2-(methylamino)-1H-imidazol-1-yl)methyl)isoquinolin-1(2H)-one (108)

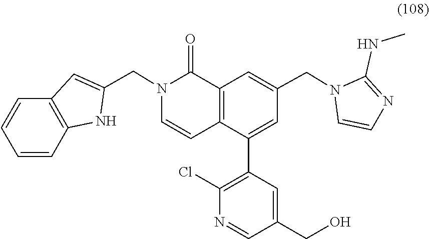 [SMILES: CNc1nccn1Cc1cc(-c2cc(CO)cnc2Cl)c2ccn(Cc3cc4ccccc4[nH]3)c(=O)c2c1]

2-((1H-indol-2-yl)methyl)-5-(2-chloro-5-(hydroxymethyl)pyridin-3-yl)-7-((2-(methylamino)-1H-imidazol-1-yl)methyl)isoquinolin-1(2H)-one (108) was synthesized following a similar procedure as described in Example 107, except tert-butyl 2-(hydroxymethyl)-4-methyl-1H-indole-1-carboxylate was replaced with tert-butyl 2-(hydroxymethyl)-1H-indole-1-carboxylate. ¹H NMR (400 MHz, Methanol-d4) δ 8.49 (d, J=2.3 Hz, 1H), 8.30 (d, J=2.0 Hz, 1H), 7.81 (d, J=2.4 Hz, 1H), 7.58 (d, J=2.0 Hz, 1H), 7.48 (dd, J=8.0, 6.4 Hz, 2H), 7.32 (d, J=8.1 Hz, 1H), 7.08 (ddd, J=8.1, 7.0, 1.2 Hz, 1H), 7.04 (s, 2H), 7.02-6.91 (m, 1H), 6.43 (s, 1H), 6.22 (d, J=7.6 Hz, 1H), 5.44 (d, J=15.0 Hz, 1H), 5.38-5.26 (m, 3H), 4.72 (s, 2H), 3.03 (s, 3H). LCMS: [M+H]⁺=525.0.

### Example 109: Synthesis of 2-(1-(6-fluoro-5-methoxypyridin-3-yl)ethyl)-7-((2-(methylamino)-1H-imidazol-1-yl)methyl)phthalazin-1(2H)-one (109)

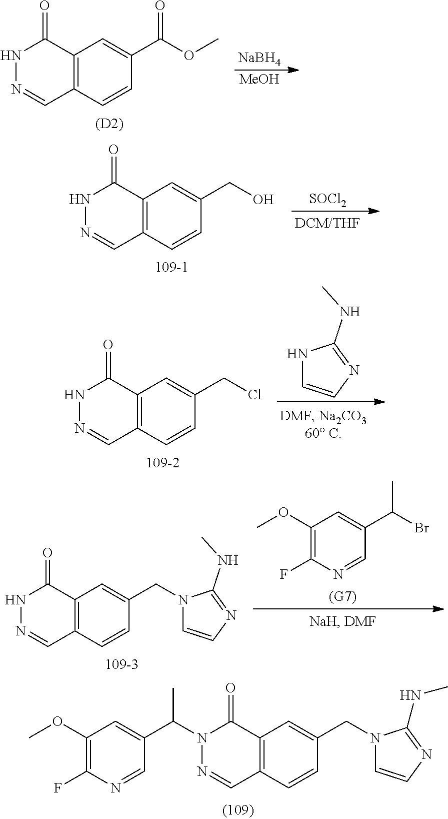

Step 1: To a solution of intermediate (D2) (100 mg, 0.49 mmol) in methanol (2 mL) was added NaBH₄ (186 mg, 4.9 mmol). The reaction mixture was stirred at 25° C. for 5 hrs. Water (0.5 mL) was then added. The reaction mixture was filtered and washed with methanol (5 mL×3). The combined organic layers were concentrated to afford 7-(hydroxymethyl)phthalazin-1(2H)-one (109-1) (100 mg, crude) as a white solid. LCMS: [M+H]⁺=177.2.

Step 2: To a solution of compound 109-1 (120 mg, 0.68 mmol) in DCM (3 mL) and THF (1 mL) was added DIEA (3 drops) and SOCl₂ (811 mg, 6.8 mmol) dropwise at 25° C. The reaction mixture was stirred at 25° C. for 2 hours. The reaction mixture was concentrated to afford compound 7-(chloromethyl)phthalazin-1(2H)-one (109-2) (200 mg, crude) as a yellow solid. LCMS: [M+H]⁺=195.2.

Step 3: To a solution of 109-2 (200 mg, 0.68 mmol, crude) and N-methyl-1H-imidazol-2-amine (137 mg, 0.88 mmol) in DMF (5 mL) was added Na₂CO₃ (722 mg, 6.8 mmol) at 25° C. under N₂. The reaction mixture was stirred at 60° C. for 12 hours. The reaction mixture was filtered and washed with methanol (10 mL×3). The organic phase was concentrated and purified by Prep-TLC (Dichloromethane/Methanol=10:1, 5% Et₃N) to afford 7-((2-(methylamino)-1H-imidazol-1-yl)methyl)phthalazin-1(2H)-one (109-3) (78 mg, 0.31 mmol) as a red solid. LCMS: [M+H]⁺=256.3.

Step 4: To a solution of compound 109-3 (40 mg, 0.16 mmol) in DMF (1 mL) was added NaH (8 mg, 0.19 mmol, 60% purity in oil) at 0° C. under N₂. The mixture was then stirred at 0° C. for 20 mins under N₂. A solution of intermediate (G7) (44 mg, 0.19 mmol) in DMF (0.5 mL) was added to the above mixture at 0° C. The reaction was stirred at 0° C. for 1 hour. The mixture was quenched with water (0.2 mL) and the resulting mixture was purified by prep-HPLC (Waters xbridge 150*25 mmm*5 um, gradient: 28-49% of B, A=water (0.05% NH₃.H₂O)-ACN, B=acetonitrile, flow rate: 25 mL/min) to afford 2-(1-(6-fluoro-5-methoxypyridin-3-yl)ethyl)-7-((2-(methylamino)-1H-imidazol-1-yl)methyl)phthalazin-1(2H)-one (109) as a pink solid. ¹H-NMR (400 MHz, Methanol-d₄) δ ppm 8.42 (s, 1H), 8.12 (s, 1H), 7.88 (d, J=8.2 Hz, 1H), 7.80-7.75 (m, 1H), 7.70-7.61 (m, 2H), 6.69 (d, J=1.8 Hz, 1H), 6.65 (d, J=1.6 Hz, 1H), 6.42 (q, J=7.1 Hz, 1H), 5.18 (s, 2H), 3.91 (s, 3H), 2.86 (s, 3H), 1.85 (d, J=7.0 Hz, 3H). LC-MS: [M+H]⁺=409.3.

### Example 110: 3-(1-(6-fluoro-5-methoxypyridin-3-yl)ethyl)-6-((2-(methylamino)-1H-imidazol-1-yl)methyl)quinazolin-4(3H)-one (110)

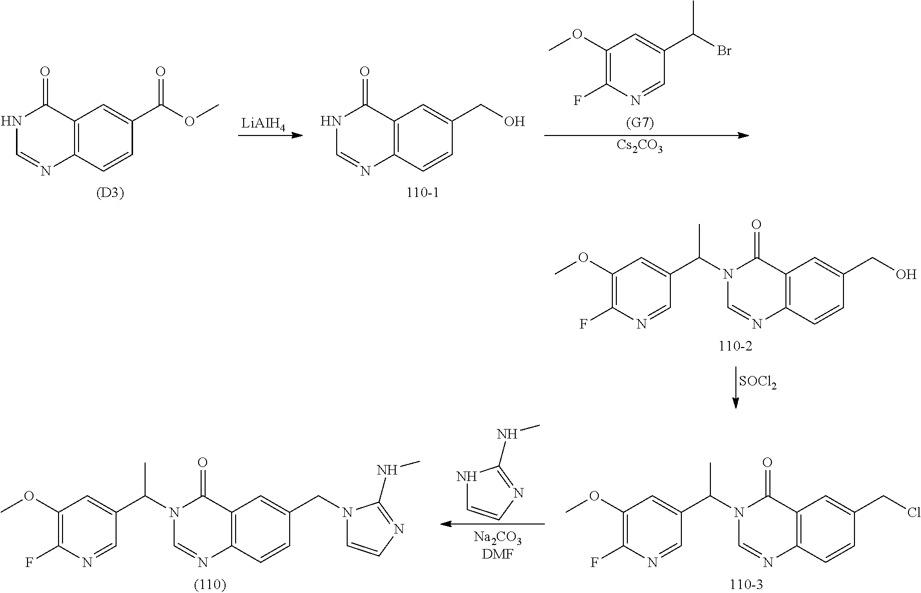

Step 1: To a solution of intermediate (D3) (400 mg, 1.96 mmol) in THF (8 mL) was added LiAlH₄ (297 mg, 7.84 mmol) 0° C. The mixture was stirred at 0° C. for 2 hrs. The reaction was quenched with water (0.3 ml), aq. NaOH (15%, 0.3 mL) was added and the mixture was filtered. The aqueous phase was extracted with ethyl acetate/ethanol=5/1 (10 mL) and concentrated, and the residue was purified by column chromatography (1%-20% methanol in EtOAc) to afford the 6-(hydroxymethyl)quinazolin-4(3H)-one (110-1) (270 mg, 80%) as a white solid. ¹H NMR (400 MHz, Methanol-d₄) δ 8.22 (s, 1H), 8.08 (s, 1H), 7.84 (dd, J=2.0, 8.4 Hz, 1H), 7.69 (d, J=8.4 Hz, 1H), 4.75 (s, 2H). LC-MS: [M+H]⁺: 177.2

Step 2: To a solution of compound 110-1 (100 mg, 0.568 mmol) in DMF (5 mL) was added intermediate (G7) (159 mg, 0.681 mmol) and Cs₂CO₃ (1.85 g, 5.68 mmol). The mixture was stirred at 20° C. for 1 hr. The mixture was diluted with water (40 ml), extracted with ethyl acetate/ethanol=5/1 (30 mL×5) and concentrated. The residue was purified by column chromatography (20%-100% ethyl acetate in hexane) to afford 3-(1-(6-fluoro-5-methoxypyridin-3-yl)ethyl)-6-(hydroxymethyl)quinazolin-4(3H)-one (110-2) (350 mg, 81% yield) as yellow oil. ¹H-NMR (400 MHz, CDCl₃) δ ppm 8.31 (d, J=1.5 Hz, 1H), 7.99 (s, 1H), 7.86-7.79 (m, 2H), 7.75-7.68 (m, 1H), 7.22 (dd, J=2.0, 9.5 Hz, 1H), 6.33 (q, J=7.2 Hz, 1H), 4.86 (br d, J=3.1 Hz, 2H), 3.93 (d, J=1.0 Hz, 1H), 3.87 (s, 3H), 1.91 (d, J=7.2 Hz, 3H). LC-MS: [M+H]⁺=330.2.

Step 3: To a solution of 110-2 (100 mg, 0.303 mmol) and DIPEA (cat.) in dichloromethane/tetrahydrofuran (0.9/0.3 mL) was added SOCl₂ (361 mg, 3.04 mmol) at 0° C. The mixture was stirred at 20° C. for 2 hrs. The mixture was concentrated to afford 6-(chloromethyl)-3-(1-(6-fluoro-5-methoxypyridin-3-yl)ethyl)quinazolin-4(3H)-one (110-3) (150 mg, crude).

Step 4: To a solution of compound 110-3 (150 mg, crude) and N-methyl-1H-imidazol-2-amine (41 mg, 0.42 mmol) in DMF (3 mL) was added Na₂CO₃ (128 mg, 1.21 mmol) at 20° C. The mixture was stirred at 60° C. for 18 hrs under the N₂ atmosphere. The reaction was filtrated and purified by Prep-HPLC (Column: Gemini 150*25 10 u, gradient: 13-27% B (A=water (0.225% FA), B=acetonitrile), flow rate: 28 mL/min) to afford 3-(1-(6-fluoro-5-methoxypyridin-3-yl)ethyl)-6-((2-(methylamino)-1H-imidazol-1-yl)methyl)quinazolin-4(3H)-one (110) as a brown gum. ¹H-NMR (400 MHz, Methanol-d₄) δ ppm 8.45 (s, 1H), 8.03 (s, 1H), 7.81-7.69 (m, 3H), 7.62 (dd, J=2.0, 9.7 Hz, 1H), 6.99-6.91 (m, 2H), 6.15 (q, J=7.1 Hz, 1H), 5.21 (s, 2H), 3.91 (s, 3H), 2.98 (s, 3H), 1.94 (d, J=7.3 Hz, 3H). LC-MS: [M+H]⁺=409.3

### Example 111: Synthesis of (S)-4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile (111

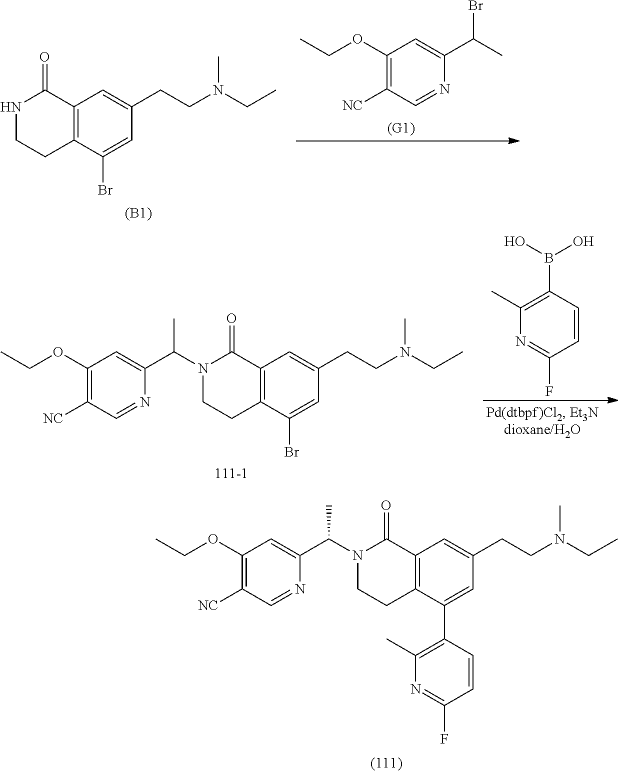

Step 1: To a solution of intermediate (B1) (68 g, 218.5 mmol, 1.0 eq.) in DMF (1000 mL) was added NaH (13.1 g, 327.8 mmol, 1.5 eq, 60% purity in oil) at 0° C. under nitrogen atmosphere. The mixture was stirred at 0° C. for 20 mins under nitrogen atmosphere. A solution of compound (G1) (66.9 g, 262.2 mmol, 1.2 eq) in DMF (300 mL) was added to the above mixture at 0° C. The reaction was stirred at 0° C. for 1 hr. The mixture was quenched with water (3000 mL) and then extracted with ethyl acetate (1000 ml×3). The combined organic phases were concentrated under reduced pressure. The residue was purified by column chromatography eluting with ethyl acetate/methanol (gradient: 2%-10% methanol, 1% Et₃N) to afford 6-(1-(5-bromo-7-(2-(ethyl(methyl)amino)ethyl)-1-oxo-3,4-dihydroisoquinolin-2(1H)-yl)ethyl)-4-ethoxynicotinonitrile (111-1) (51 g, 48% yield) as oil. ¹H NMR (400 MHz, CDCl₃): δ 8.63 (s, 1H), 7.85 (d, J=1.7 Hz, 1H), 7.65 (d, J=1.7 Hz, 1H), 7.18 (s, 1H), 5.94 (q, J=7.1 Hz, 1H), 4.35-4.26 (m, 2H), 4.10 (q, J=7.1 Hz, 1H), 3.69-3.59 (m, 1H), 3.58-3.50 (m, 1H), 3.11-2.92 (m, 2H), 2.88-2.78 (m, 2H), 2.69-2.60 (m, 2H), 2.54 (q, J=7.2 Hz, 2H), 2.32 (s, 3H), 1.66 (d, J=7.1 Hz, 3H), 1.47 (t, J=7.0 Hz, 3H), 1.10 (t, J=7.2 Hz, 3H).

Step 2: To a solution of compound 111-1 (78 g, 160.69 mmol, 1.0 eq) and compound (6-fluoro-2-methylpyridin-3-yl)boronic acid (29.9 g, 192.8 mmol, 1.2 eq) in dioxane/H₂O (1000 mL/200 mL) was added Et₃N (65 g, 642.8 mmol, 4.0 eq) and Pd(dtbpf)Cl₂ (10.5 g, 16.07 mmol, 0.1 eq). The mixture was stirred at 40° C. for 2 hrs under N₂. LCMS showed the reaction was complete. The reaction mixture was concentrated and the residue purified by by silica gel column chromatography eluting with ethyl acetate/methanol (gradient: 5-10% of methanol, 1% Et₃N) to afford the crude product (100 g). The crude was further purified by prep-HPLC (Phenomenex luna C18 250 mm*100 mm*10 um; Gradient: 10-40% of B, A=0.225% FA in water, B=acetonitrile, flow rate: 400 mL/min). The resulting solution was concentrated under reduced pressure, adjusted to pH 8 with sat NaHCO₃ and extracted with acetate ethyl (1000 mL×3). The combined organic phases were dried over Na₂SO₄ and concentrated under reduced pressure to afford a racemate product as a yellow oil. The racemate was separated by SFC (column: CHIRALCEL OD (150*4.6 mm I.D. 3 um), Method: OD_3_EtOH_DEA_5_40_25 mL) to afford (S)-4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile (111) as a yellow solid. ¹H NMR (400 MHz, Methanol-d₄) δ 8.60 (d, J=3.0 Hz, 1H), 7.94 (d, J=1.9 Hz, 1H), 7.68 (dt, J=12.9, 8.1 Hz, 1H), 7.27 (d, J=1.9 Hz, 1H), 7.18 (d, J=3.5 Hz, 1H), 6.96 (ddd, J=8.6, 6.0, 2.7 Hz, 1H), 5.98 (qd, J=7.2, 2.8 Hz, 1H), 4.31 (qd, J=7.1, 2.9 Hz, 2H), 3.62-3.40 (m, 2H), 2.93-2.83 (m, 2H), 2.73-2.49 (m, 6H), 2.32 (s, 3H), 2.22 (d, J=17.6 Hz, 3H), 1.64 (dd, J=7.2, 2.1 Hz, 3H), 1.47 (t, J=7.0 Hz, 3H), 1.10 (t, J=7.2 Hz, 3H). LC-MS: 516 [M+H]⁺.

### Example 122: (S)-6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile (122)

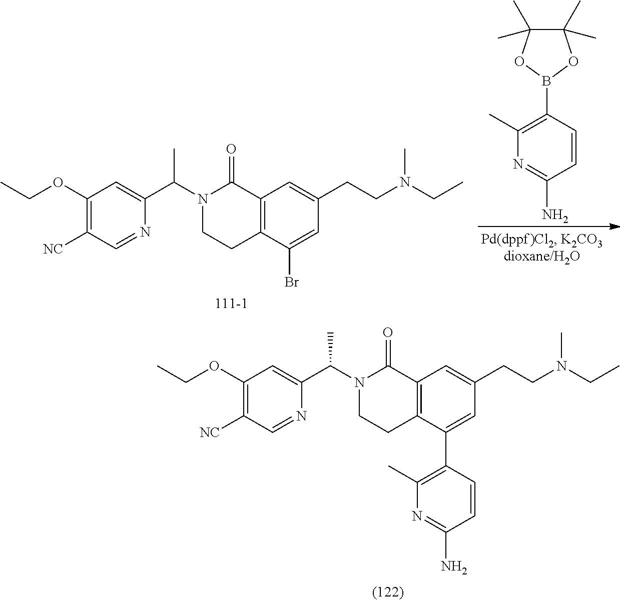

To a solution of compound 111-1 (14.5 g, 29.87 mmol, 1.0 eq) in dioxane (160 mL)/H2O (40 mL) was added compound 6-methyl-5-(4,4,5,5-tetramethyl-1,3,2-dioxaborolan-2-yl)pyridin-2-amine (11.2 g, 47.79 mmol, 1.6 eq) and K₂CO₃ (12.38 g, 89.61 mmol, 3.0 eq). Pd(dppf)Cl2 (2.44 g, 2.981 mmol, 0.1 eq) was added to the reaction mixture at 20° C. under N₂. The mixture was stirred at 80° C. for 2 hrs. The mixture was concentrated and the residue was purified by column chromatography on silica gel eluting with acetate ethyl/methanol (30%˜35% of methanol, 5% Et₃N) to give the crude compound (20 g). The crude compound was purified by Prep-HPLC (Column: Phenomenex luna C18 250 mm*100 mm*10 um, Condition: A=water (0.225% FA)-ACN, 5˜35% of B, B=acetonitrile, flow rate: 400 ml/min, Gradient Time (min): 25 min) to give a racemate product (10 g, 65.32% yield) as white solid. The racemate was separated by SFC (column: DAICEL CHIRALCEL OD (250 mm*30 mm, 10 um); Gradient Time: 4.1 min; 700 min; Condition: 0.1% NH₃H₂O MEOH; Flow Rate: 70 g/ml; 40% of B) to afford (S)-6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile 122 (4.1 g, 41% yield) as a yellow solid. ¹H NMR (400 MHz, Methanol-d₄) δ ppm 8.61 (d, J=2.8 Hz, 1H), 7.96 (d, J=1.8 Hz, 1H), 7.92 (d, J=2.6 Hz, 1H), 7.37-7.28 (m, 1H), 7.19 (s, 1H), 6.87 (dd, J=2.8, 12.4 Hz, 1H), 6.00 (dq, J=4.0, 7.2 Hz, 1H), 4.32 (q, J=7.0 Hz, 2H), 3.63-3.42 (m, 2H), 3.23-3.05 (m, 4H), 2.84 (s, 3H), 2.74-2.51 (m, 2H), 2.10 (d, J=17.0 Hz, 3H), 1.66 (dd, J=1.0, 7.2 Hz, 3H), 1.48 (t, J=7.0 Hz, 3H), 1.33 (t, J=7.2 Hz, 3H), 1.20 (s, 1H). LC-MS: [M+H]⁺=513

### Example 134: (S)-6-(1-(5-(1,3-dimethyl-1H-pyrazol-4-yl)-7-(2-(ethyl(methyl)amino)ethyl)-1-oxo-3,4-dihydroisoquinolin-2(1H)-yl)ethyl)-4-ethoxynicotinonitrile (134)

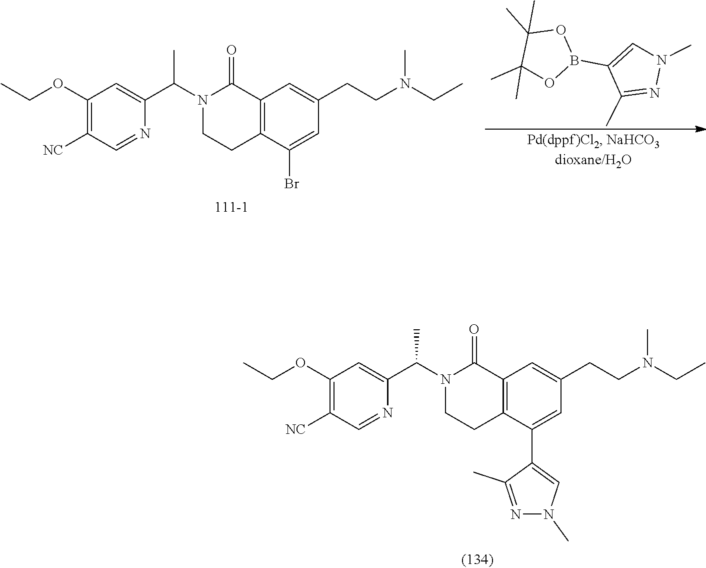

The mixture of 111-1 (20 mg, 0.041 mmol), 1,3-dimethyl-4-(4,4,5,5-tetramethyl-1,3,2-dioxaborolan-2-yl)-1H-pyrazole (13.73 mg, 0.062 mmol), SODIUM BICARBONATE (10.38 mg, 0.124 mmol) and PdCl2(dppf) (15.07 mg, 0.021 mmol) in Dioxane (Volume: 3 mL, Ratio: 3.75)/H2O (Volume: 0.8 mL, Ratio: 1.000) was stirred for 2 hr at 95° C. under N2 atmosphere. The mixture was concentrated and the residue was purified by column chromatography on silica gel eluting with acetate ethyl/methanol (30%˜35% of methanol, 5% Et₃N) to give the racemate compound, which was separated by SFC (column: CHIRALCEL OD (150*4.6 mm I.D. 3 um), Method: OD_3_EtOH_DEA_5_40_25 mL) to afford (S)-6-(1-(5-(1,3-dimethyl-1H-pyrazol-4-yl)-7-(2-(ethyl(methyl)amino)ethyl)-1-oxo-3,4-dihydroisoquinolin-2(1H)-yl)ethyl)-4-ethoxynicotinonitrile (134) as a yellow solid. ¹H NMR (400 MHz, Methanol-d₄) δ 8.62 (s, 1H), 7.88 (d, J=1.9 Hz, 1H), 7.54 (s, 1H), 7.37 (d, J=1.9 Hz, 1H), 7.19 (s, 1H), 5.97 (q, J=7.1 Hz, 1H), 4.37-4.25 (m, 2H), 3.86 (s, 3H), 3.56-3.42 (m, 2H), 3.28-3.22 (m, 2H), 3.20-3.01 (m, 4H), 2.81 (s, 5H), 2.13 (s, 3H), 1.66 (d, J=7.1 Hz, 3H), 1.48 (t, J=7.0 Hz, 3H), 1.31 (t, J=7.3 Hz, 3H). LC-MS: [M+H]⁺=501.
Following a similar procedure to that of Example 111 (unless otherwise specified). The following compounds were prepared from their corresponding precursors.

| Ex. | Name/Structure | ¹HNMR/LC-MS | Precursors |
|---|---|---|---|
| 112 | 6-((5-(2-chloro-5-methylpyridin-3- | ¹H NMR (400 MHz, Methanol-d₄) | Step 1: |
| | yl)-7-(2-(ethyl(methyl)amino)ethyl)- | δ ppm 8.60 (s, 1H), 8.53 (s, 1H), | intermediate (G1) |
| | 1-oxo-3,4-dihydroisoquinolin-2(1H)- | 8.28 (d, J = 1.7 Hz, 1H), 7.99 (d, | was replaced with |
| | yl)methyl)-4-ethoxynicotinonitrile | J = 1.7 Hz, 1H), 7.65 (d, J = 1.8 Hz, | intermediate (G2) |
| | 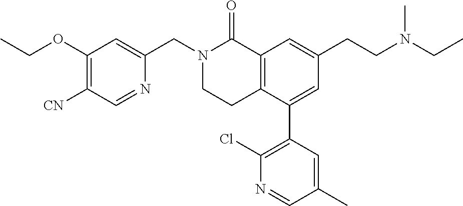 | 1H), 7.35 (d, J = 1.8 Hz, 1H), 7.18 (s, 1H), 4.89 (s, 2H), 4.31 (q, J = 7.0 Hz, 2H), 3.72-3.61 (m, 2H), 3.04 (br s, 4H), 2.96-2.88 (m, 2H), 2.87-2.78 (m, 1H), 2.77- 2.67 (m, 1H), 2.63 (s, 3H), 2.40 (s, 3H), 1.48 (t, J = 7.0 Hz, 3H), 1.23 (t, J = 7.2 Hz, 3H). LC-MS: [M + H]⁺ = 518.2 | Step 2: (6-fluoro- 2-methylpyridin-3- yl)boronic acid was replaced with (2-chloro-5- methylpyridin-3- yl)boronic acid |
| 113 | (S)-5-(1,3-dimethyl-1H-pyrazol-4- | ¹H NMR (400 MHz, Methanol-d₄) | Step 1: |
| | yl)-7-(2-(ethyl(methyl)amino)ethyl)- | δ ppm 8.24 (d, J = 3.2 Hz, 1H), | intermediate (G1) |
| | 2-(1-(5-fluoro-4-methoxypyridin-2- | 7.89 (d, J = 1.9 Hz, 1H), 7.53 (s, | was replaced with |
| | yl)ethyl)-3,4-dihydroisoquinolin- | 1H), 7.30 (d, J = 1.9 Hz, 1H), 7.20 | intermediate (G9) |
| | 1(2H)-one | (d, J = 6.9 Hz, 1H), 6.03 (q, J = | Step 2: (6-fluoro- |
| | 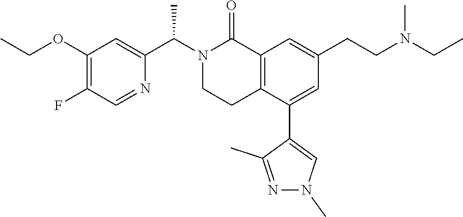 | 7.1 Hz, 1H), 3.97 (s, 3H), 3.87 (s, 3H), 3.50 (ddd, J = 13.3, 8.7, 5.1 Hz, 1H), 3.40-3.34 (m, 1H), 2.96- 2.66 (m, 6H), 2.59 (q, J = 7.2 Hz, 2H), 2.36 (s, 3H), 2.13 (s, 3H), 1.64 (d, J = 7.1 Hz, 3H), 1.13 (t, J = 7.2 Hz, 3H). LC-MS: [M + H]⁺ = 480 | 2-methylpyridin-3- yl)boronic acid was replaced with (1,3-dimethyl-1H- pyrazol-4- yl)boronic acid |
| 114 | (S)-6-(1-(5-(2,5-dimethylpyridin-3- | ¹H NMR (400 MHz, Methanol-d₄) | Step 1: |
| | yl)-7-(2-(ethyl(methyl)amino)ethyl)- | δ ppm 8.61 (d, J = 2.8 Hz, 1H), | intermediate (G1) |
| | 1-oxo-3,4-dihydroisoquinolin-2(1H)- | 8.29 (q, J = 0.9 Hz, 1H), 7.93 (d, | Step 2: (6-fluoro- |
| | yl)ethyl)-4-ethoxynicotinonitrile | J = 1.9 Hz, 1H), 7.42 (dd, J = 12.7, | 2-methylpyridin-3- |
| | 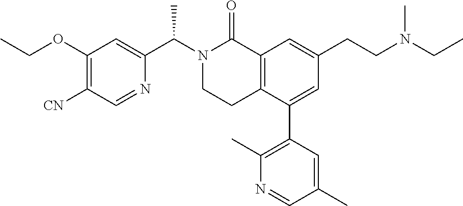 | 2.1 Hz, 1H), 7.22 (dd, J = 27.1, 2.6 Hz, 2H), 5.98 (qd, J = 7.1,3.3 Hz, 1H), 4.38-4.24 (m, 2H), 3.60- 3.38 (m, 2H), 2.96-2.85 (m, 2H), 2.78-2.68 (m, 2H), 2.67- 2.47 (m, 4H), 2.40-2.31 (m, 6H), 2.23 (d, J = 17.5 Hz, 3H), 1.64 (dd, J = 7.2, 2.2 Hz, 3H), 1.47 (t, J = 7.0 Hz, 3H), 1.11 (t, J = 7.2 Hz, 3H). LC-MS: [M + H]⁺ = 512.3 | yl)boronic acid was replaced with (2,5- dimethylpyridin-3- yl)boronic acid |
| 115 | (S)-4-ethoxy-6-(1-(7-(2- | ¹H NMR (400 MHz, Methanol-d₄) | Step 1: |
| | (ethyl(methyl)amino)ethyl)-5-(1-(2- | δ ppm 8.62 (s, 1H), 7.84 (s, 1H), | intermediate (G1) |
| | hydroxyethyl)-3-methyl-1H-pyrazol- | 7.57 (s, 1H), 7.29 (s, 1H), 7.18 (s, | Step 2: (6-fluoro- |
| | 4-yl)-1-oxo-3,4-dihydroisoquinolin- | 1H), 6.00 (q, J = 7.0 Hz, 1H), 4.33 | 2-methylpyridin-3- |
| | 2(1H)-yl)ethyl)nicotinonitrile | (q, J = 7.0 Hz, 2H), 4.19(t, J = 5.4 | yl)boronic acid |
| | 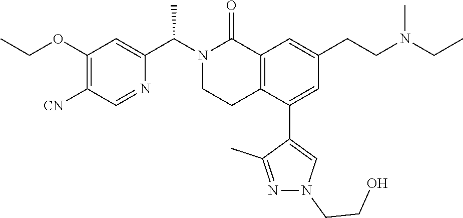 | Hz, 2H), 3.90 (t, J = 5.4 Hz, 2H), 3.58-3.39 (m, 2H), 2.90-2.77 (m, 4H), 2.70-2.62(m, 2H), 2.55 (q, J = 7.2 Hz, 2H), 2.32 (s, 3H), 2.14 (s, 3H), 1.66 (d, J = 7.2 Hz, 3H), 1.47 (t, J = 7.0 Hz, 3H), 1.10 (t, J = 7.2 Hz,3H). LC-MS: [M + H]⁺ = 531 | was replaced with (1-(2- hydroxyethyl)-3- methyl-1H- pyrazol-4- yl)boronic acid |
| 116 | (S)-4-ethoxy-6-(1-(7-(2- | ¹H NMR (400 MHz, Methanol-d₄) | Step 1: |
| | (ethyl(methyl)amino)ethyl)-5-(3- | δ ppm 8.63 (s, 1H), 7.86 (d, J = | intermediate (G1) |
| | methyl-1H-pyrazol-4-yl)-1-oxo-3,4- | 1.9 Hz, 1H), 7.52 (s, 1H), 7.31 (d, | Step 2: (6-fluoro- |
| | dihydroisoquinolin-2(1H)- | J = 1.9 Hz, 1H), 7.20 (s, 1H), 6.00 | 2-methylpyridin-3- |
| | yl)ethyl)nicotinonitrile | (q, J = 7.1 Hz, 1H), 4.33 (q, J = | yl)boronic acid |
| | 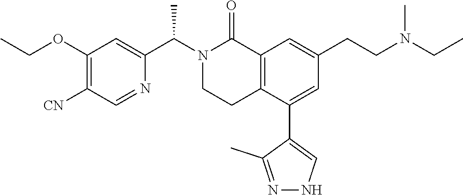 | 7.0 Hz, 2H), 3.61-3.41 (m, 2H), 2.95-2.78 (m, 4H), 2.76-2.67 (m, 2H), 2.59 (q, J = 7.2 Hz, 2H), 2.36 (s, 3H), 2.21 (s, 3H), 1.67 (d, J = 7.1 Hz, 3H), 1.49 (t, J = 7.0 Hz, 3H), 1.13 (t, J = 7.2 Hz, 3H). LC-MS: [M + H]⁺ = 487 | was replaced with (3-methyl-1H- pyrazol-4- yl)boronic acid |
| 117 | (S)-4-ethoxy-6-(1-(7-(2- | ¹H NMR (400 MHz, Methanol-d₄) | Step 1: |
| | (ethyl(methyl)amino)ethyl)-5-(2- | δ ppm 8.61 (d, J = 1.9 Hz, 1H), | intermediate (G1) |
| | fluoro-3-methylpyridin-4-yl)-1-oxo- | 8.05 (t, J = 5.3 Hz, 1H), 7.96 (d, | Step 2: (6-fluoro- |
| | 3,4-dihydroisoquinolin-2(1H)- | J = 1.9 Hz, 1H), 7.26 (d, J = 1.9 Hz, | 2-methylpyridin-3- |
| | yl)ethyl)nicotinonitrile | 1H), 7.18 (d, J = 4.0 Hz, 1H), 7.11 | yl)boronic acid |
| | 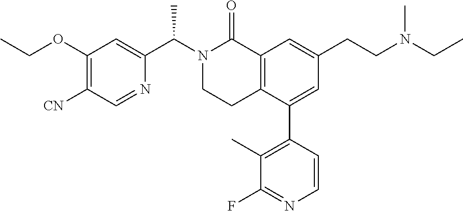 | (dd, J = 11.8, 5.1 Hz, 1H), 5.98 (tt, J = 7.3, 4.0 Hz, 1H), 4.31 (qd, J = 7.0, 2.8 Hz, 2H), 3.60-3.40 (m, 2H), 2.90 (dd, J = 10.0, 6.2 Hz, 2H), 2.74-2.49 (m, 6H), 2.33 (s, 3H), 2.02 (dd, J = 17.1, 1.4 Hz, 3H), 1.64 (dd, J = 7.1, 1.7 Hz, 3H), 1.47 (t, J = 7.0 Hz, 3H), 1.10 (t, J = 7.2 Hz, 3H). LC-MS: [M + H]⁺ = 516 | was replaced with (3-fluoropyridin-4- yl)boronic acid |
| 118 | (S)-6-(1-(5-(2-chloro-5- | ¹H NMR (400 MHz, Methanol-d₄) | Step 1: |
| | methylpyridin-3-yl)-7-(2- | δ ppm 8.60 (d, J = 9.1 Hz, 1H), | intermediate (G1) |
| | (ethyl(methyl)amino)ethyl)-1-oxo- | 8.25 (d, J = 2.3 Hz, 1H), 7.95 (s, | Step 2: (6-fluoro- |
| | 3,4-dihydroisoquinolin-2(1H)- | 1H), 7.62 (dd, J = 13.2, 2.4 Hz, | 2-methylpyridin-3- |
| | yl)ethyl)-4-ethoxynicotinonitrile | 1H), 7.28 (d, J = 2.0 Hz, 1H), 7.18 | yl)boronic acid |
| | 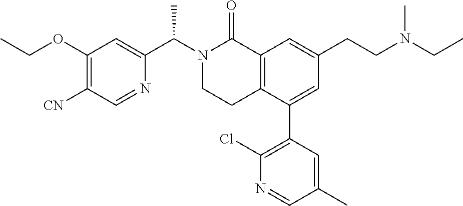 | (d, J = 2.8 Hz, 1H), 5.98 (qd, J = 7.2, 2.3 Hz, 1H), 4.31 (qd, J = 7.1, 2.7 Hz, 2H), 3.62-3.39 (m, 2H), 2.89 (dd, J = 10.2, 6.2 Hz, 2H), 2.80-2.46 (m, 6H), 2.41- 2.28 (m, 6H), 1.65 (dd, J = 7.2, 5.3 Hz, 3H), 1.47 (t, J = 7.0 Hz, 3H), 1.10 (t, J = 7.2 Hz, 3H). LC-MS: [M + H]⁺ = 532 | was replaced with (2-chloro-5- methylpyridin-3- yl)boronic acid |
| 119 | (S)-6-(1-(5-(2- | ¹H NMR (400 MHz, Methanol-d₄) | Step 1: |
| | (difluoromethyl)pyridin-3-yl)-7-(2- | δ ppm 8.71 (dt, J = 4.7, 1.8 Hz, | intermediate (G1) |
| | (ethyl(methyl)amino)ethyl)-1-oxo- | 1H), 8.61 (s, 1H), 7.97 (t, J = 1.2 | Step 2: (6-fluoro- |
| | 3,4-dihydroisoquinolin-2(1H)- | Hz, 1H), 7.79 (ddd, J = 14.0, 7.9, | 2-methylpyridin-3- |
| | yl)ethyl)-4-ethoxynicotinonitrile | 1.6 Hz, 1H), 7.64 (td, J = 7.9, 4.7 | yl)boronic acid |
| | 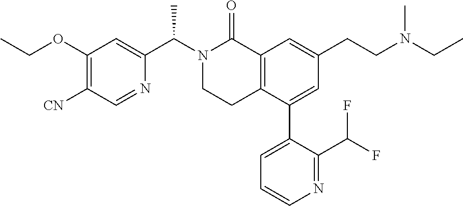 | Hz, 1H), 7.30 (t, J = 1.5 Hz, 1H), 7.18 (d, J = 4.6 Hz, 1H), 6.55 (td, J = 53.9, 16.4 Hz, 1H), 5.98 (qd, J = 7.1, 3.4 Hz, 1H), 4.30 (pd, J = 6.2, 5.3, 3.2 Hz, 2H), 3.57-3.36 (m, 2H), 2.96-2.85 (m, 2H), 2.75- 2.43 (m, 6H), 2.32 (s, 3H), 1.64 (dd, J = 7.1, 3.2 Hz, 3H), 1.47 (td, J = 7.0, 2.2 Hz, 3H), 1.10 (t, J = 7.2 Hz, 3H). LC-MS: [M + H]⁺ = 534 | was replaced with (2- (difluoromethyl) pyridin-3- yl)boronic acid |
| 120 | (S)-5-(2-chloro-5- | ¹H NMR (400 MHz, Methanol-d₄) | Step 1: |
| | (hydroxymethyl)pyridin-3-yl)-2-(1-(4- | δ ppm 8.39 (d, J = 2.3 Hz, 1H), | intermediate (G1) |
| | ethoxy-5-fluoropyridin-2-yl)ethyl)-7- | 8.21 (dd, J = 9.5, 3.2 Hz, 1H), | was replaced with |
| | (2-(ethyl(methyl)amino)ethyl)-3,4- | 7.98 (d, J = 1.9 Hz, 1H), 7.74 (dd, | intermediate (G5) |
| | dihydroisoquinolin-1(2H)-one | J = 16.2, 2.3 Hz, 1H), 7.29 (d, J = | Step 2: (6-fluoro- |
| | 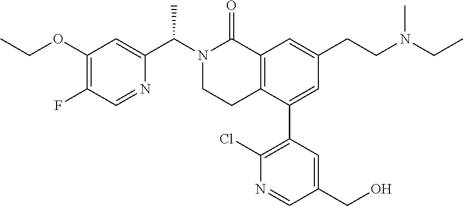 [SMILES: CCOc1cc([C@H](C)N2CCc3c(cc(CCN(C)CC)cc3-c3cc(CO)cnc3Cl)C2=O)ncc1F] | 1.9 Hz, 1H), 7.14 (dd, J = 6.9, 4.3 Hz, 1H), 5.99 (tt, J = 7.0, 4.0 Hz, 1H), 4.68 (d, J = 7.4 Hz, 2H), 4.19 (ddtd, J = 9.6, 6.9, 4.9, 2.3 Hz, 2H), 3.55-3.33 (m, 2H), 2.90 (dd, J = 10.3, 6.1 Hz, 2H), 2.75- 2.47 (m, 6H), 2.32 (s, 3H), 1.61 (t, J = 6.8 Hz, 3H), 1.43 (td, J = 7.0, 1.4 Hz, 3H), 1.11 (d, J = 7.2 Hz, 3H). LC-MS: [M + H]⁺ = 541.3 | 2-methylpyridin-3- yl)boronic acid was replaced with (2-chloro-5- (hydroxymethyl) pyridin-3- yl)boronic acid |
| 121 | (S)-2-(1-(5-chloro-4-ethoxypyridin- | ¹H NMR (400 MHz, Methanol-d₄) | Step 1: |
| | 2-yl)ethyl)-5-(1,3-dimethyl-1H- | δ ppm 8.36 (s, 1H), 7.89 (d, J = | intermediate (G1) |
| | pyrazol-4-yl)-7-(2- | 1.9 Hz, 1H), 7.53 (s, 1H), 7.30 (d, | was replaced with |
| | (ethyl(methyl)amino)ethyl)-3,4- | J = 1.9 Hz, 1H), 7.12 (s, 1H), 6.02 | intermediate (G11) |
| | dihydroisoquinolin-1(2H)-one | (q, J = 7.1 Hz, 1H), 4.25 (qd, J = | Step 2: (6-fluoro- |
| | 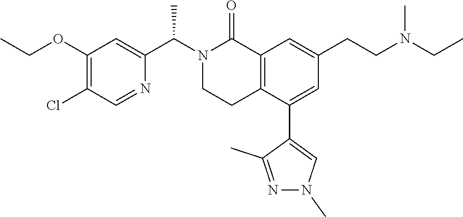 [SMILES: CCOc1cc([C@H](C)N2CCc3c(cc(CCN(C)CC)cc3-c3cn(C)nc3C)C2=O)ncc1Cl] | 7.0, 2.0 Hz, 2H), 3.88 (s, 3H), 3.51 (ddd, J = 12.6, 8.8, 5.1 Hz, 1H), 3.42-3.38 (m, 1H), 2.96-2.85 (m, 2H), 2.83-2.65 (m, 4H), 2.58 (q, J = 7.2 Hz, 2H), 2.35 (s, 3H), 2.14 (s, 3H), 1.66 (d, J = 7.1 Hz, 3H), 1.48 (t, J = 7.0 Hz, 3H), 1.13 (t, J = 7.2 Hz, 3H). LC-MS: [M + H]⁺ = 510.3 | 2-methylpyridin-3- yl)boronic acid was replaced with (1,3-dimethyl-1H- pyrazol-4- yl)boronic acid |
| 123 | (S)-5-(1,3-dimethyl-1H-pyrazol-4- | ¹H NMR (400 MHz, Methanol-d₄) | Step 1: |
| | yl)-7-(2-(ethyl(methyl)amino)ethyl)- | δ ppm 7.88 (s, 1H), 7.73 (s, 1H), | intermediate (G1) |
| | 2-(1-(6-fluoro-5-methoxypyridin-3- | 7.56-7.43 (m, 2H), 7.29 (s, 1H), | was replaced with |
| | yl)ethyl)-3,4-dihydroisoquinolin- | 6.10 (d, J = 8.0 Hz, 1H), 3.87 (dd, | intermediate (G7) |
| | 1(2H)-one | J = 22.8, 4.4 Hz, 6H), 3.46 (t, J = | Step 2: (6-fluoro- |
| | 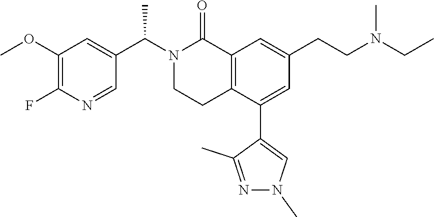 [SMILES: CCN(C)CCc1cc2c(c(-c3cn(C)nc3C)c1)CCN([C@@H](C)c1cnc(F)c(OC)c1)C2=O] | 8.2 Hz, 1H), 3.15 (d, J = 11.0 Hz, 1H), 2.97-2.65 (m, 6H), 2.57 (d, J = 7.9 Hz, 2H), 2.34 (d, J = 4.5 Hz, 3H), 2.11 (d, J = 4.4 Hz, 3H), 1.65 (d, J = 6.3 Hz, 3H), 1.12 (t, J = 7.3 Hz, 3H). LC-MS: [M + H]⁺ = 480 | 2-methylpyridin-3- yl)boronic acid was replaced with (1,3-dimethyl-1H- pyrazol-4- yl)boronic acid |
| 124 | (S)-6-(1-(5-(2-chloro-5- | ¹H NMR (400 MHz, Methanol-d₄) | Step 1: |
| | (hydroxymethyl)pyridin-3-yl)-7-(2- | δ 8.60 (d, J = 10.3 Hz, 1H), 8.39 | intermediate (G1) |
| | (ethyl(methyl)amino)ethyl)-1-oxo- | (d, J = 2.3 Hz, 1H), 7.96 (t, J = 1.5 | Step 2: (6-fluoro- |
| | 3,4-dihydroisoquinolin-2(1H)- | Hz, 1H), 7.75 (dd, J = 13.7, 2.4 | 2-methylpyridin-3- |
| | yl)ethyl)-4-ethoxynicotinonitrile | Hz, 1H), 7.30 (d, J = 1.9 Hz, 1H), | yl)boronic acid |
| | 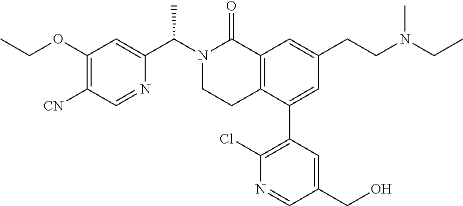 | 7.18 (d, J = 3.3 Hz, 1H), 5.98 (qd, J = 7.1, 2.9 Hz, 1H), 4.69 (d, J = 5.5 Hz, 2H), 4.31 (ttd, J = 7.0, 4.1, 2.0 Hz, 2H), 3.68-3.39 (m, 2H), 2.96-2.85 (m, 2H), 2.79-2.51 (m, 6H), 2.32 (s, 3H), 1.65 (dd, J = 7.2, 5.9 Hz, 3H), 1.47 (td, J = 7.0, 1.5 Hz, 3H), 1.10 (t, J = 7.2 Hz, 3H). LC-MS: [M + H]⁺ = 548 | was replaced with (2-chloro-5- (hydroxymethyl) pyridin-3- yl)boronic acid |
| 125 | (S)-4-ethoxy-6-(1-(7-(2- | ¹H NMR (400 MHz, Methanol-d₄) | Step 1: |
| | (ethyl(methyl)amino)ethyl)-1-oxo-5- | δ 8.61 (d, J = 4.3 Hz, 1H), 7.91 (d, | intermediate (G1) |
| | (1,4,5-trimethyl-6-oxo-1,6- | J = 1.7 Hz, 1H), 7.34 (d, J = 14.3 | Step 2: (6-fluoro- |
| | dihydropyridin-3-yl)-3,4- | Hz, 1H), 7.25 (s, 1H), 7.18 (d, | 2-methylpyridin-3- |
| | dihydroisoquinolin-2(1H)- | J = 5.6 Hz, 1H), 6.03-5.93 (m, | yl)boronic acid |
| | yl)ethyl)nicotinonitrile | 1H), 4.36-4.25 (m, 2H), 3.57 (d, | was replaced with |
| | 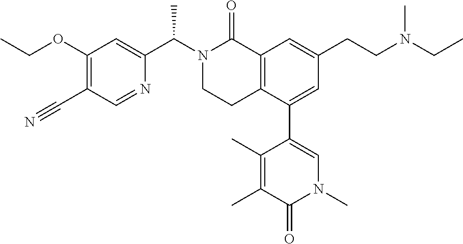 [SMILES: CCOc1cc([C@H](C)N2CCc3c(cc(CCN(C)CC)cc3-c3cn(C)c(=O)c(C)c3C)C2=O)ncc1C#N] | J = 6.4 Hz, 3H), 3.53 (dd, J = 4.2, 8.7 Hz, 1H), 3.49-3.41 (m, 1H), 2.92-2.84 (m, 2H), 2.77-2.65 (m, 3H), 2.65-2.59(m, 1H), 2.56 (q, J = 7.2 Hz, 2H), 2.33 (s, 3H), 2.15 (d, J = 5.0 Hz, 3H), 1.92 (d, J = 18.6 Hz, 3H), 1.65(dd, J = 3.6, 7.2 Hz, 3H), 1.47 (dt, J = 1.0, 7.0 Hz, 3H), 1.11 (t, J = 7.2 Hz, 3H). LC-MS: [M + H]⁺ = 542 | (1,4,5-trimethyl-6- oxo-1,6- dihydropyridin-3- yl)boronic acid |
| 126 | (S)-6-(1-(5-(3-(difluoromethyl)-1- | ¹H NMR (400 MHz, Methanol-d₄) | Step 1: |
| | methyl-1H-pyrazol-4-yl)-7-(2- | δ 8.64 (s, 1H), 8.03 (d, J = 1.9 Hz, | intermediate (G1) |
| | (ethyl(methyl)amino)ethyl)-1-oxo- | 1H), 7.44 (d, J = 1.9 Hz, 1H), 7.22 | Step 2: (6-fluoro- |
| | 3,4-dihydroisoquinolin-2(1H)- | (s, 1H), 6.77 (t, J = 55.0 Hz, 1H), | 2-methylpyridin-3- |
| | yl)ethyl)-4-ethoxynicotinonitrile | 6.55 (d, J = 1.2 Hz, 1H), 6.01 (q, | yl)boronic acid |
| | 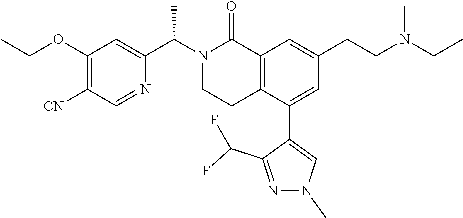 | J = 7.1 Hz, 1H), 4.34 (q, J = 7.0 Hz, 2H), 3.72 (s, 3H), 3.65-3.45 (m, 2H), 2.99-2.87 (m, 2H), 2.82- 2.67 (m, 4H), 2.58 (q, J = 7.2 Hz, 2H), 2.35 (s, 3H), 1.68 (d, J = 7.1 Hz, 3H), 1.50 (t, J = 7.0 Hz, 3H), 1.13 (t, J = 7.2 Hz, 3H). LC-MS: [M + H]⁺ = 537 | was replaced with (3- (difluoromethyl)-1- methyl-1H- pyrazol-4- yl)boronic acid |
| 127 | (S)-4-ethoxy-6-(1-(7-(2- | ¹H NMR (400 MHz, Methanol-d₄) | Step 1: |
| | (ethyl(methyl)amino)ethyl)-5-(1- | δ 8.62 (s, 1H), 7.84 (d, J = 1.6 Hz, | intermediate (G1) |
| | ethyl-3-methyl-1H-pyrazol-4-yl)-1- | 1H), 7.56 (s, 1H), 7.29 (d, J = 1.8 | Step 2: (6-fluoro- |
| | oxo-3,4-dihydroisoquinolin-2(1H)- | Hz, 1H), 7.18 (s, 1H), 5.98 (q, | 2-methylpyridin-3- |
| | yl)ethyl)nicotinonitrile | J = 7.2 Hz, 1H), 4.31 (q, J = 7.0 | yl)boronic acid |
| | 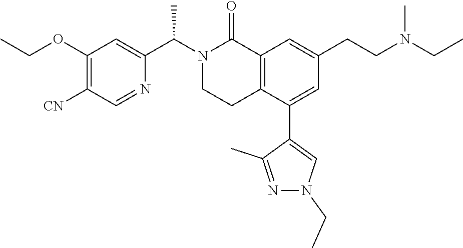 | Hz, 2H), 4.14 (q, J = 7.2 Hz, 2H), 3.59-3.39 (m, 2H), 2.92- 2.85 (m, 2H), 2.83-2.76 (m, 2H), 2.75-2.69 (m, 2H), 2.61 (q, J = 7.2 Hz, 2H), 2.37 (s, 3H), 2.13 (s, 3H), 1.65 (d, J = 7.2 Hz, 3H), 1.46 (m, 6H), 1.12(t, J = 7.2 Hz, 3H) LC-MS: [M + H]⁺ = 515 | was replaced with (1-ethyl-3-methyl- 1H-pyrazol-4- yl)boronic acid |
| 128 | (S)-6-(1-(5-(2-(difluoromethyl)-5- | ¹H NMR (400 MHz, Methanol-d₄) | Step 1: |
| | methylpyridin-3-yl)-7-(2- | δ 8.60 (s, 1H), 8.54 (q, J = 2.8, | intermediate (G1) |
| | (ethyl(methyl)amino)ethyl)-1-oxo- | 2.2 Hz, 1H), 7.97 (t, J = 1.6 Hz, | Step 2: (6-fluoro- |
| | 3,4-dihydroisoquinolin-2(1H)- | 1H), 7.66-7.55 (m, 1H), 7.28 (d, | 2-methylpyridin-3- |
| | yl)ethyl)-4-ethoxynicotinonitrile | J = 1.8 Hz, 1H), 7.18 (d, J = 5.6 | yl)boronic acid |
| | 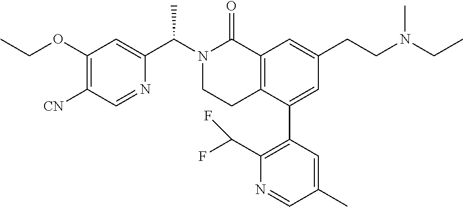 | Hz, 1H), 6.49 (td, J = 54.0, 16.8 Hz, 1H), 5.98 (qd, J = 7.1, 3.4 Hz, 1H), 4.30 (qdd, J = 8.2, 5.6, 2.9 Hz, 2H), 3.62-3.36 (m, 2H), 2.89 (dd, J = 10.1, 6.1 Hz, 2H), 2.77- 2.47 (m, 6H), 2.45 (d, J = 7.2 Hz, 3H), 2.34 (s, 3H), 1.64 (dd, J = 7.2, 3.3 Hz, 3H), 1.47 (td, J = 7.0, 3.6 Hz, 3H), 1.10 (t, J = 7.2 Hz, 3H). LC-MS: [M + H]⁺ = 548 | was replaced with (2- (difluoromethyl)-5- methylpyridin-3- yl)boronic acid |
| 129 | 6-((5-(1,3-dimethyl-1H-pyrazol-4-yl)- | ¹H NMR (400 MHz, Methanol-d₄) | Step 1: |
| | 7-(2-(ethyl(methyl)amino)ethyl)-1- | δ 8.60 (s, 1H), 8.52 (br s, 1H), | intermediate (G1) |
| | oxo-3,4-dihydroisoquinolin-2(1H)- | 7.90 (d, J = 1.6 Hz, 1H), 7.55 (s, | Step 2: (6-fluoro- |
| | yl)methyl)-4-ethoxynicotinonitrile | 1H), 7.38 (d, J = 1.8 Hz, 1H), 7.19 | 2-methylpyridin-3- |
| | 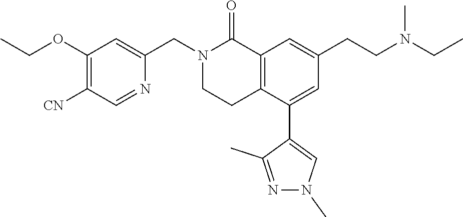 | (s, 1H), 4.89 (s, 2H), 4.31 (m, 2H), 3.87 (s, 3H), 3.65 (t, J = 6.6 Hz, 2H), 3.29-3.24 (m, 2H), 3.16 (m, 2H), 3.12-3.05 (m, 2H), 2.92 (t, J = 6.6 Hz, 2H), 2.82 (s, 3H), 2.14 (s, 3H), 1.48 (t, J = 7.0 Hz, 3H), 1.32 (t, J = 7.3 Hz, 3H). LC-MS: [M + H]⁺ = 487 | yl)boronic acid was replaced with (1,3-dimethyl-1H- pyrazol-4- yl)boronic acid |
| 130 | 4-ethoxy-6-((7-(2- | ¹H NMR (400 MHz, Methanol-d₄) | Step 1: |
| | (ethyl(methyl)amino)ethyl)-5-(6- | δ 8.72 (s, 1H), 8.23 (s, 1H), 7.84 | intermediate (G1) |
| | fluoro-2-methylpyridin-3-yl)-1-oxo- | (s, 1H), 7.77 (t, J = 8.3 Hz, 1H), | Step 2: (6-fluoro- |
| | 3,4-dihydroisoquinolin-2(1H)- | 7.28 (s, 1H), 7.23 (s, 1H), 7.08 | 2-methylpyridin-3- |
| | yl)methyl)nicotinonitrile | (dd, J = 2.7, 8.2 Hz, 1H), 4.90- | yl)boronic acid |
| | 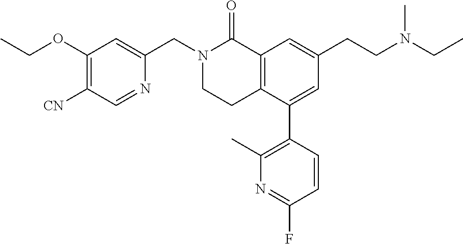 | 4.76 (m, 2H), 4.30 (q, J = 7.0 Hz, 2H), 3.55 (br t, J = 6.4 Hz, 2H), 2.85-2.74 (m, 2H), 2.73-2.53 (m, 4H), 2.48-2.44 (m, 2H), 2.24(s, 3H), 2.20 (s, 3H), 1.37 (t, J = 7.0 Hz, 3H), 0.97 (t, J = 7.1 Hz, 3H) LC-MS: [M + H]⁺ = 502 | was replaced with (6-fluoro-2- methylpyridin-3- yl)boronic acid |
| 131 | (S)-6-(1-(5-(1,4-dimethyl-6-oxo-1,6- | ¹H NMR (400 MHz, Methanol-d₄) | Step 1: |
| | dihydropyridin-3-yl)-7-(2- | δ 8.61 (s, 1H), 7.91 (s, 1H), 7.46 | intermediate (G1) |
| | (ethyl(methyl)amino)ethyl)-1-oxo- | (d, J = 14.3 Hz, 1H), 7.27 (d, J = 1.3 | Step 2: (6-fluoro- |
| | 3,4-dihydroisoquinolin-2(1H)- | Hz, 1H), 7.19 (d, J = 2.7 Hz, 1H), | 2-methylpyridin-3- |
| | yl)ethyl)-4 ethoxynicotinonitrile | 6.50 (d, J = 5.3 Hz, 1H), 6.02- | yl)boronic acid |
| | 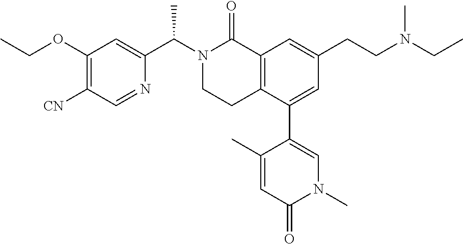 | 5.93 (m, 1H), 4.32 (q, J = 7.0 Hz, 2H), 3.56 (d, J = 6.6 Hz, 4H), 2.93- 2.83 (m, 2H), 2.81-2.59 (m, 4H), 2.54 (q, J = 7.2 Hz, 2H), 2.32 (s, 3H), 1.95 (d, J = 17.6 Hz, 3H), 1.65 (d, J = 7.1 Hz, 3H), 1.47 (t, J = 7.0 Hz, 3H), 1.10 (t, J = 7.2 Hz, 3H) LC-MS: [M + H]⁺ = 528 | was replaced with (1,4-dimethyl-6- oxo-1,6- dihydropyridin-3- yl)boronic acid |
| 132 | 2-((4-ethoxy-5-fluoropyridin-2- | ¹H NMR (400 MHz, Methanol-d₄) | Step 1: |
| | yl)methyl)-7-(2- | δ 8.49 (br s, 1H), 8.20 (d, J = 3.4 | intermediate (G1) |
| | (ethyl(methyl)amino)ethyl)-5-(6- | Hz, 1H), 8.03 (d, J = 1.6 Hz, 1H), | was replaced with |
| | fluoro-2-methylpyridin-3-yl)-3,4- | 7.71 (t, J = 8.0 Hz, 1H), 7.38 (d, | (G6) |
| | dihydroisoquinolin-1(2H)-one | J = 1.6 Hz, 1H), 7.24 (dd, J = 6.8, | Step 2: (6-fluoro- |
| | 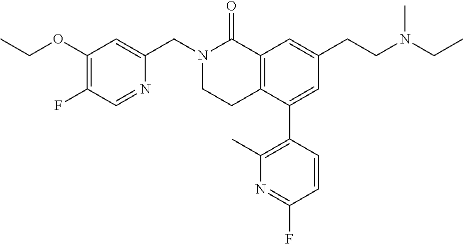 [SMILES: CCOc1cc(CN2CCc3c(cc(CCN(C)CC)cc3-c3ccc(F)nc3C)C2=O)ncc1F] | 8.6 Hz, 1H), 7.16 (d, J = 7.0 Hz, 1H), 6.99 (dd, J = 2.6, 8.4 Hz, 1H), 6.70 (dd, J = 3.0,8.5 Hz, 1H), 4.81 (s, 2H), 4.21 (q, J = 7.0 Hz, 2H), 3.64-3.56 (m, 2H), 3.43-3.35 (m, 2H), 3.26 (q, J = 7.6 Hz, 3H), 3.19-3.11(m, 2H), 2.91 (s, 3H), 2.79-2.49 (m, 2H), 2.24 (s, 3H), 1.44 (t, J = 7.0 Hz, 3H), 1.36 (t, J = 7.0 Hz, 3H) LC-MS: [M + H]⁺ = 495. | 2-methylpyridin-3- yl)boronic acid |
| 133 | 6-((5-(5-chloro-2-methylpyridin-4- | ¹H NMR (400 MHz, Methanol-d₄) | Step 1: |
| | yl)-7-(2-(ethyl(methyl)amino)ethyl)- | δ 8.60 (s, 1H), 8.54 (s, 1H), 8.00 | intermediate (G1) |
| | 1-oxo-3,4-dihydroisoquinolin-2(1H)- | (d, J = 1.7 Hz, 1H), 7.33 (d, J = 1.7 | was replaced with |
| | yl)methyl)-4-ethoxynicotinonitrile | Hz, 1H), 7.29 (s, 1H), 7.18 (s, | (G2) |
| | 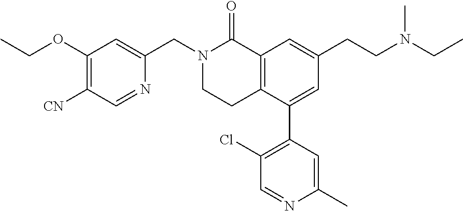 | 1H), 4.89 (s, 2H), 4.31 (q, J = 7.1 Hz, 2H), 3.73-3.61 (m, 2H), 3.00 (br s, 4H), 2.90-2.69 (m, 4H), 2.57 (s, 6H), 1.48 (t, J = 7.0 Hz, 3H), 1.21 (t, J = 7.3 Hz, 3H). LC-MS: [M + H]⁺ = 518 | Step 2: (6-fluoro- 2-methylpyridin-3- yl)boronic acid was replaced with (5-chloro-2- methylpyridin-4- yl)boronic acid |
| 135 | 6-((5-(1,4-dimethyl-6-oxo-1,6- | ¹H NMR (400 MHz, Methanol-d₄) | Step 1: |
| | dihydropyridin-3-yl)-7-(2- | δ 8.59 (s, 1H), 7.93 (d, J = 2.0 Hz, | intermediate (G1) |
| | (ethyl(methyl)amino)ethyl)-1-oxo- | 1H), 7.49 (s, 1H), 7.30 (d, J = 2.0 | was replaced with |
| | 3,4-dihydroisoquinolin-2(1H)- | Hz, 1H), 7.18 (s, 1H), 6.52 (s, | (G2) |
| | yl)methyl)-4-ethoxynicotinonitrile | 1H), 4.88 (d, J = 2.8 Hz, 2H), 4.30 | Step 2: (6-fluoro- |
| | 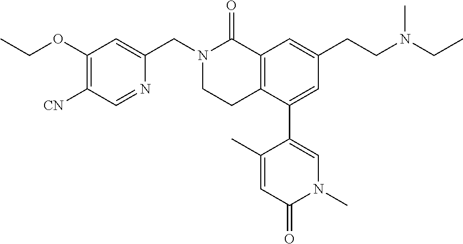 | (q, J = 7.0 Hz, 2H), 3.70-3.62 (m, 2H), 3.56 (s, 3H), 2.98-2.78 (m, 5H), 2.71 (dq, J = 14.6, 7.3, 6.8 Hz, 3H), 2.44 (s, 3H), 1.96 (s, 3H), 1.47 (t, J = 7.0 Hz, 3H), 1.15 (t, J = 7.2 Hz, 3H). LC-MS: [M + H]⁺ = 514 | 2-methylpyridin-3- yl)boronic acid was replaced with (1,4-dimethyl-6- oxo-1,6- dihydropyridin-3- yl)boronic acid |
| 136 | 6-(1-(5-(5-chloro-2-methoxypyridin- | ¹H NMR (400 MHz, Methanol-d₄) | Step 1: |
| | 4-yl)-7-(2- | δ 8.61 (d, J = 7.0 Hz, 1H), 8.22 | intermediate (G1) |
| | (ethyl(methyl)amino)ethyl)-1-oxo- | (d, J = 4.9 Hz, 1H), 7.96 (d, J = | Step 2: (6-fluoro- |
| | 3,4-dihydroisoquinolin-2(1H)- | 1.9 Hz, 1H), 7.26 (t, J = 1.6 Hz, | 2-methylpyridin-3- |
| | yl)ethyl)-4-ethoxynicotinonitrile | 1H), 7.18 (d, J = 4.5 Hz, 1H), 6.81- | yl)boronic acid |
| | 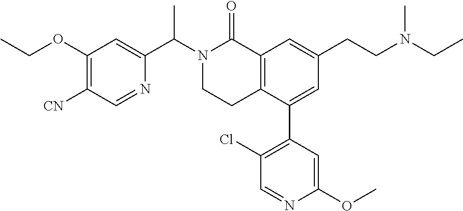 | 6.71 (m, 1H), 5.98 (qd, J = 7.1, 1.9 Hz, 1H), 4.37-4.25 (m, 2H), 3.93 (d, J = 3.0 Hz, 3H), 3.62- 3.42 (m, 2H), 2.97-2.86 (m, 2H), 2.78-2.54 (m, 6H), 2.36 (s, 3H), 1.65 (dd, J = 7.2, 4.2 Hz, 3H), 1.47 (td, J = 7.1, 1.3 Hz, 3H), 1.11 (t, J = 7.2 Hz, 3H). LC-MS: [M + H]⁺ = 548. | was replaced with (5-chloro-2- methoxypyridin-4- yl)boronic acid |
| 137 | 7-(2-(ethyl(methyl)amino)ethyl)-5- | ¹H NMR (400 MHz, Methanol-d₄) | Step 1: |
| | (6-fluoro-2-methylpyridin-3-yl)-2-((6- | δ 8.02 (d, J = 1.7 Hz, 1H), 7.77- | intermediate (G1) |
| | fluoro-5-methoxypyridin-3- | 7.66 (m, 2H), 7.57 (dd, J = 1.8, 9.8 | was replaced with |
| | yl)methyl)-3,4-dihydroisoquinolin- | Hz, 1H), 7.33 (d, J = 1.7 Hz, 1H), | (G8) |
| | 1(2H)-one | 6.99 (dd, J = 2.5, 8.3 Hz, 1H), 4.83- | Step 2: (6-fluoro- |
| | 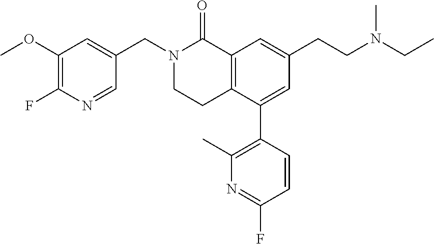 [SMILES: CCN(C)CCc1cc2c(c(-c3ccc(F)nc3C)c1)CCN(Cc1cnc(F)c(OC)c1)C2=O] | 4.73 (m, 2H), 3.92 (s, 3H), 3.56 (t, J = 6.7 Hz, 2H), 3.04-2.88 (m, 4H), 2.79(q, J = 7.1 Hz, 2H), 2.75- 2.56 (m, 2H), 2.53 (s, 3H), 2.24 (s, 3H), 1.20 (t, J = 7.2 Hz, 3H). LC-MS: [M + H]⁺ = 481. | 2-methylpyridin-3- yl)boronic acid was replaced with (6-fluoro-2- methylpyridin-3- yl)boronic acid |
| 138 | (S)-6-(1-(5-(5-amino-2- | ¹H NMR (400 MHz, Methanol-d₄) | Step 1: |
| | (trifluoromethyl)pyridin-3-yl)-7-(2- | δ 8.61 (d, J = 2.1 Hz, 1H), 8.01 (t, | intermediate (G1) |
| | (ethyl(methyl)amino)ethyl)-1-oxo- | J = 2.0 Hz, 1H), 7.94 (s, 1H), 7.27 | Step 2: (6-fluoro- |
| | 3,4-dihydroisoquinolin-2(1H)- | (s, 1H), 7.18 (d, J = 8.5 Hz, 1H), | 2-methylpyridin-3- |
| | yl)ethyl)-4-ethoxynicotinonitrile | 6.82 (dd, J = 2.4, 14.5 Hz, 1H), | yl)boronic acid |
| | 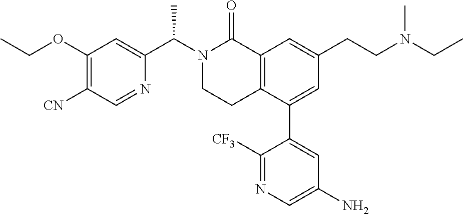 | 6.02-5.92 (m, 1H), 4.31 (q, J = 6.5 Hz, 2H), 3.57 (ddd, J = 4.7, 8.1, 12.8 Hz, 1H), 3.52-3.47 (m, 1H), 3.40 (ddd, J = 5.1, 7.9, 12.9 Hz, 1H), 3.01-2.84 (m, 4H), 2.77 (q, J = 7.1 Hz, 2H), 2.72-2.62 (m, 1H), 2.61-2.54 (m, 1H), 2.51 (s, 3H), 1.65 (dd, J = 2.6, 7.1 Hz, 3H), 1.47 (dt, J = 4.3, 7.0 Hz, 3H), 1.18 (t, J = 7.3 Hz, 3H). LC-MS: [M + H]⁺ = 567. | was replaced with (5-amino-2- (trifluoromethyl) pyridin-3- yl)boronic acid |
| 139 | (S)-4-ethoxy-6-(1-(7-(2- | ¹H NMR (400 MHz, Methanol-d₄) | Step 1: |
| | (ethyl(methyl)amino)ethyl)-5-(2- | δ 8.55 (d, J = 3.3 Hz, 1H), 8.39 | intermediate (G1) |
| | methylpyridin-3-yl)-1-oxo-3,4- | (dd, J = 5.0, 1.7 Hz, 1H), 7.88 (d, | Step 2: (6-fluoro- |
| | dihydroisoquinolin-2(1H)- | J = 1.8 Hz, 1H), 7.54 (ddd, J = | 2-methylpyridin-3- |
| | yl)ethyl)nicotinonitrile | 12.6, 7.7, 1.8 Hz, 1H), 7.29 (dt, | yl)boronic acid |
| | 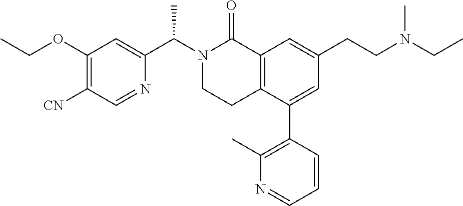 | J = 7.7, 5.2 Hz, 1H), 7.21 (d, J = 1.9 Hz, 1H), 7.12 (d, J = 4.0 Hz, 1H), 5.92 (qd, J = 7.1, 3.0 Hz, 1H), 4.25 (qd, J = 7.0, 3.4 Hz, 2H), 3.51-3.33 (m, 2H), 2.84 (dd, J = 10.2, 6.1 Hz, 2H), 2.69-2.61 (m, 2H), 2.51 (p, J = 7.1, 6.5 Hz, 4H), 2.28 (s, 3H), 2.23 (d, J = 17.2 Hz, 3H), 1.58 (dd, J = 7.1, 2.4 Hz, 3H), 1.41 (td, J = 7.0, 0.9 Hz, 3H), 1.05 (t, J = 7.2 Hz, 3H). LC-MS: [M + H]⁺ = 498. | was replaced with (2-methylpyridin-3- yl)boronic acid |
| 140 | 6-((5-(2,5-dimethylpyridin-3-yl)-7-(2- | ¹H NMR (400 MHz, Methanol-d₄) | Step 1: |
| | (ethyl(methyl)amino)ethyl)-1-oxo- | δ 8.59 (s, 1H), 8.31 (d, J = 1.6 Hz, | intermediate (G1) |
| | 3,4-dihydroisoquinolin-2(1H)- | 1H), 7.96 (d, J = 1.7 Hz, 1H), 7.45 | was replaced with |
| | yl)methyl)-4-ethoxynicotinonitrile | (d, J = 1.6 Hz, 1H), 7.30(d, J = 1.7 | (G2) |
| | 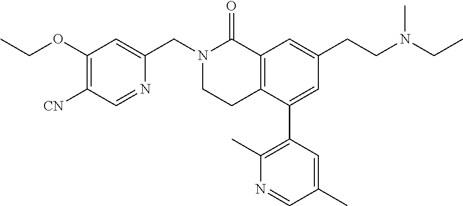 | Hz, 1H), 7.18 (s, 1H), 4.89 (d, J = 2.7 Hz, 2H), 4.31 (q, J = 7.0 Hz, 2H), 3.65 (t, J = 6.6 Hz, 2H), 2.99(s, 4H), 2.84 (q, J = 6.9 Hz, 2H), 2.78-2.63 (m, 2H), 2.57 (s, 3H), 2.37 (s, 3H), 2.25 (s, 3H), 1.48 (t, J = 7.0 Hz, 3H), 1.21 (t, J = 7.3 Hz, 3H). LC-MS: [M + H]⁺ = 498. | Step 2: (6-fluoro- 2-methylpyridin-3- yl)boronic acid was replaced with (2,5- dimethylpyridin-3- yl)boronic acid |
| 141 | 4-ethoxy-6-(1-(7-(2- | ¹H NMR (400 MHz, Methanol-d₄) | Step 1: |
| | (ethyl(methyl)amino)ethyl)-5-(2- | δ 8.61 (d, J = 2.0 Hz, 1H), 8.06 (t, | intermediate (G1) |
| | fluoro-3-methylpyridin-4-yl)-1-oxo- | J = 5.2 Hz, 1H), 7.98 (d, J = 1.9 | Step 2: (6-fluoro- |
| | 3,4-dihydroisoquinolin-2(1H)- | Hz, 1H), 7.29 (d, J = 1.9 Hz, 1H), | 2-methylpyridin-3- |
| | yl)ethyl)nicotinonitrile | 7.19 (d, J = 3.9 Hz, 1H), 7.11 (dd, | yl)boronic acid |
| | 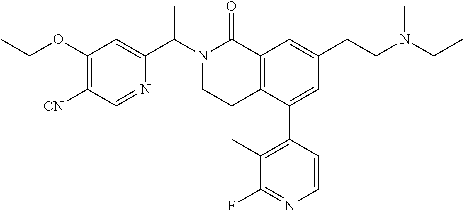 | J = 11.3, 5.1 Hz, 1H), 6.04-5.92 (m, 1H), 4.37-4.26 (m, 2H), 3.52 (d, J = 28.0 Hz, 2H), 2.98 (s, 4H), 2.81 (s, 2H), 2.59 (d, J = 33.9 Hz, 5H), 2.08-1.96 (m, 3H), 1.65 (dd, J = 7.2, 1.7 Hz, 3H), 1.47 (t, J = 6.9 Hz, 3H), 1.19 (t, J = 7.2 Hz, 3H). LC-MS: [M + H]⁺ = 516. | was replaced with (2-fluoro-3- methylpyridin-4- yl)boronic acid |
| 142 | (S)-4-ethoxy-6-(1-(7-(2- | ¹H NMR (400 MHz, Methanol-d₄) | Step 1: |
| | (ethyl(methyl)amino)ethyl)-5-(5- | δ 8.62 (s, 1H), 8.39 (d, J = 1.7 Hz, | intermediate (G1) |
| | fluoro-2-methylpyridin-4-yl)-1-oxo- | 1H), 7.99 (d, J = 1.8 Hz, 1H), 7.40 | Step 2: (6-fluoro- |
| | 3,4-dihydroisoquinolin-2(1H)- | (d, J = 1.8 Hz, 1H), 7.30 (d, J = | 2-methylpyridin-3- |
| | yl)ethyl)nicotinonitrile | 6.0 Hz, 1H), 7.19 (s, 1H), 5.98 (q, | yl)boronic acid |
| | 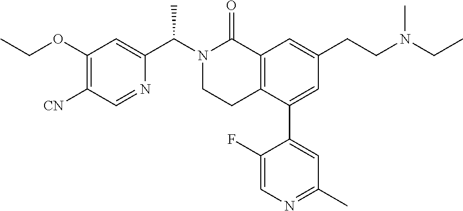 | J = 7.1 Hz, 1H), 4.31 (q, J = 7.0 Hz, 2H), 3.60-3.40 (m, 2H), 2.94 (dd, J = 10.3, 5.9 Hz, 2H), 2.79 (dd, J = 10.5, 5.9 Hz, 4H), 2.65 (d, J = 7.2 Hz, 2H), 2.56 (s, 3H), 2.41 (s, 3H), 1.65 (d, J = 7.1 Hz, 3H), 1.47 (t, J = 7.0 Hz, 3H), 1.14 (t, J = 7.2 Hz, 3H). LC-MS: [M + H]⁺ = 516. | was replaced with (5-fluoro-2- methylpyridin-4- yl)boronic acid |
| 143 | 4-ethoxy-6-(1-(7-(2- | ¹H NMR (400 MHz, Methanol-d₄) | Step 1: |
| | (ethyl(methyl)amino)ethyl)-5-(2- | δ 8.61 (s, 1H), 8.53 (s, 1H), 8.14 | intermediate (G1) |
| | fluoro-5-methylpyridin-4-yl)-1-oxo- | (dq, J = 4.7, 0.8 Hz, 1H), 7.99 | Step 2: (6-fluoro- |
| | 3,4-dihydroisoquinolin-2(1H)- | (dd, J = 1.9, 0.9 Hz, 1H), 7.30 (d, | 2-methylpyridin-3- |
| | yl)ethyl)nicotinonitrile | J = 1.9 Hz, 1H), 7.19 (d, J = 4.7 | yl)boronic acid |
| | 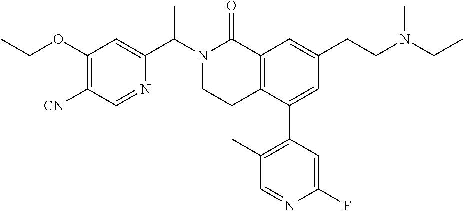 | Hz, 1H), 6.90 (dd, J = 10.1, 2.1 Hz, 1H), 5.97 (qd, J = 7.1, 2.3 Hz, 1H), 4.31 (tdd, J = 7.2, 6.5, 2.6 Hz, 2H), 3.64-3.39 (m, 2H), 3.16- 2.86 (m, 6H), 2.65 (s, 4H), 2.61- 2.44 (m, 1H), 2.05 (dt, J = 16.5, 0.9 Hz, 3H), 1.70-1.63 (m, 3H), 1.47 (t, J = 7.0 Hz, 3H), 1.24 (t, J = 7.3 Hz, 3H). LC-MS: [M + H]⁺ = 516. | was replaced with (2-fluoro-5- methylpyridin-4- yl)boronic acid |
| 144 | 6-((5-(2-(difluoromethyl)-5- | ¹H NMR (400 MHz, Methanol-d₄) | Step 1: |
| | methylpyridin-3-yl)-7-(2- | δ 8.59 (s, 1H), 8.56-8.47 (m, | intermediate (G1) |
| | (ethyl(methyl)amino)ethyl)-1-oxo- | 2H), 7.98 (d, J = 1.9 Hz, 1H), 7.63 | was replaced with |
| | 3,4-dihydroisoquinolin-2(1H)- | (dt, J = 1.7, 0.9 Hz, 1H), 7.31 (d, | (G2) |
| | yl)methyl)-4-ethoxynicotinonitrile | J = 1.9 Hz, 1H), 7.17 (s, 1H), 6.51 | Step 2: (6-fluoro- |
| | 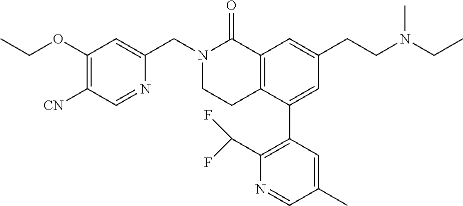 | (t, J = 54.0 Hz, 1H), 4.88 (s, 2H), 4.30 (q, J = 7.0 Hz, 2H), 3.63 (dd, J = 7.2, 6.0 Hz, 2H), 2.99-2.91 (m, 2H), 2.88-2.80 (m, 2H), 2.73 (tt, J = 14.6, 7.2 Hz, 3H), 2.61 (dt, J = 16.3, 6.2 Hz, 1H), 2.48-2.39 (m, 6H), 1.47 (t, J = 7.0 Hz, 3H), 1.15 (t, J = 7.2 Hz, 3H). LC-MS: [M + H]⁺ = 534. | 2-methylpyridin-3- yl)boronic acid was replaced with (2- (difluoromethyl)-5- methylpyridin-3- yl)boronic acid |
| 145 | (S)-5-(1,3-dimethyl-1H-pyrazol-4- | ¹H NMR (400 MHz, Methanol-d₄) | Step 1: |
| | yl)-2-(1-(4-ethoxy-5-fluoropyridin-2- | δ 8.20 (d, J = 3.3 Hz, 1H), 7.83 | intermediate (G1) |
| | yl)ethyl)-7-(2- | (d, J = 2.0 Hz, 1H), 7.48 (s, 1H), | was replaced with |
| | (ethyl(methyl)amino)ethyl)-3,4- | 7.25 (d, J = 1.9 Hz, 1H), 7.11 (d, | (G5) |
| | dihydroisoquinolin-1(2H)-one | J = 6.8 Hz, 1H), 5.96 (q, J = 7.1 Hz, | Step 2: (6-fluoro- |
| | 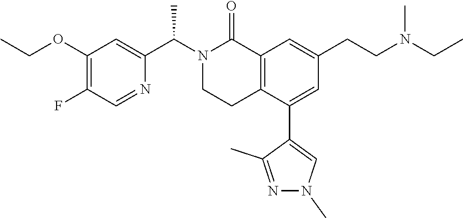 [SMILES: CCOc1cc([C@H](C)N2CCc3c(cc(CCN(C)CC)cc3-c3cn(C)nc3C)C2=O)ncc1F] | 1H), 4.17 (qd, J = 7.0, 2.1 Hz, 2H), 3.82 (s, 3H), 3.44 (ddd, J = 13.4, 8.8, 5.0 Hz, 1H), 3.34-3.29 (m, 1H), 2.87-2.62 (m, 6H), 2.53 (q, J = 7.2 Hz, 2H), 2.31 (s, 3H), 2.08 (s, 3H), 1.58 (d, J = 7.1 Hz, 3H), 1.40 (t, J = 7.0 Hz, 3H), 1.08 (t, J = 7.2 Hz, 3H). LC-MS: [M + H]⁺ = 494. | 2-methylpyridin-3- yl)boronic acid was replaced with (1,3-dimethyl-1H- pyrazol-4- yl)boronic acid |
| 146 | 5-(3-(difluoromethyl)-1-methyl-1H- | ¹H NMR (400 MHz, Methanol-d₄) | Step 1: |
| | pyrazol-4-yl)-7-(2- | δ 7.92 (d, J = 1.8 Hz, 1H), 7.67 (s, | intermediate (G1) |
| | (ethyl(methyl)amino)ethyl)-2-(1-(6- | 1H), 7.49 (dd, J = 9.7, 1.7 Hz, | was replaced with |
| | fluoro-5-methoxypyridin-3-yl)ethyl)- | 1H), 7.35 (d, J = 1.4 Hz, 1H), 7.34 | (G7) |
| | 3,4-dihydroisoquinolin-1(2H)-one | (dd, J = 97.1, 8.4 Hz, 1H), 6.66 (t, | Step 2: (6-fluoro- |
| | 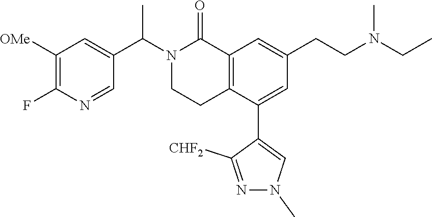 [SMILES: CCN(C)CCc1cc2c(c(-c3cn(C)nc3C(F)F)c1)CCN(C(C)c1cnc(F)c(OC)c1)C2=O] | J = 54.1 Hz, 1H), 6.10 (q, J = 7.0 Hz, 1H), 3.95 (s, 3H), 3.90 (s, 3H), 3.55-3.41 (m, 1H), 3.23- 3.09 (m, 1H), 2.95-2.82 (m, 2H), 2.81-2.65 (m, 4H), 2.56 (q, J = 7.2 Hz, 2H), 2.34 (s, 3H), 1.65 (d, J = 7.1 Hz, 3H), 1.11 (t, J = 7.2 Hz, 3H). LC-MS: [M + H]⁺ = 516. | 2-methylpyridin-3- yl)boronic acid was replaced with (3- (difluoromethyl)- 1-methyl-1H- pyrazol-4- yl)boronic acid |
| 147 | 6-(1-(5-(6-chloro-2-methylpyridin-3- | ¹H NMR (400 MHz, Methanol-d₄) | Step 1: |
| | yl)-7-(2-(ethyl(methyl)amino)ethyl)- | δ 8.61 (d, J = 3.2 Hz, 1H), 7.96 | intermediate (G1) |
| | 1-oxo-3,4-dihydroisoquinolin-2(1H)- | (d, J = 1.9 Hz, 1H), 7.56 (dd, J = | Step 2: (6-fluoro- |
| | yl)ethyl)-4-ethoxynicotinonitrile | 12.3, 8.0 Hz, 1H), 7.42-7.27 (m, | 2-methylpyridin-3- |
| | 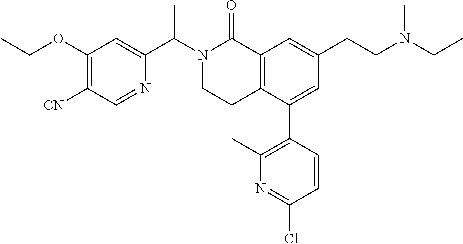 | 2H), 7.19 (d, J = 3.6 Hz, 1H), 5.97 (qd, J = 7.1, 2.9 Hz, 1H), 4.31 (qdd, J = 6.3, 3.6, 2.6 Hz, 2H), 3.62-3.39 (m, 2H), 3.03-2.85 (m, 4H), 2.77 (q, J = 7.2 Hz, 2H), 2.70-2.44 (m, 5H), 2.25 (d, J = 17.0 Hz, 3H), 1.65 (dd, J = 7.1, 2.1 Hz, 3H), 1.47 (td, J = 7.0, 0.8 Hz, 3H), 1.18 (t, J = 7.2 Hz, 3H). LC-MS: [M + H]⁺ = 532. | yl)boronic acid was replaced with (6-chloro-2- methylpyridin-3- yl)boronic acid |
| 148 | 4-ethoxy-6-(1-(7-(2- | ¹H NMR (400 MHz, Methanol-d₄) | Step 1: |
| | (ethyl(methyl)amino)ethyl)-5-(1- | δ 8.62 (s, 1H), 7.96 (d, J = 2.0 Hz, | intermediate (G1) |
| | methyl-3-(trifluoromethyl)-1H- | 1H), 7.75 (d, J = 1.1 Hz, 1H), 7.40 | Step 2: (6-fluoro- |
| | pyrazol-4-yl)-1-oxo-3,4- | (d, J = 2.0 Hz, 1H), 7.19 (s, 1H), | 2-methylpyridin-3- |
| | dihydroisoquinolin-2(1H)- | 5.96 (q, J = 7.1 Hz, 1H), 4.31 (qd, | yl)boronic acid |
| | yl)ethyl)nicotinonitrile | J = 7.0, 1.5 Hz, 2H), 3.99 (s, 3H), | was replaced with |
| | 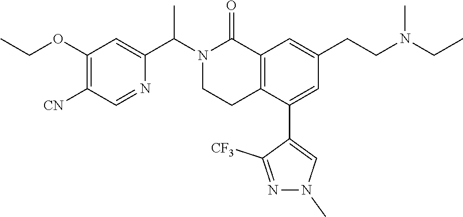 | 3.59-3.31 (m, 5H), 3.16 (dtd, J = 34.8, 12.7, 11.0, 6.5 Hz, 3H), 2.92 (s, 3H), 2.76 (t, J = 6.5 Hz, 2H), 1.65 (d, J = 7.1 Hz, 3H), 1.48 (t, J = 7.0 Hz, 3H), 1.35 (t, J = 7.3 Hz, 3H). LC-MS: [M + H]⁺ = 555. | (1-methyl-3- (trifluoromethyl)- 1H-pyrazol-4- yl)boronic acid |
| 149 | (S)-6-(1-(5-(4-(difluoromethyl)-1- | ¹H NMR (400 MHz, Methanol-d₄) | Step 1: |
| | methyl-6-oxo-1,6-dihydropyridin-3- | δ 8.60 (s, 1H), 7.95 (s, 1H), 7.65 | intermediate (G1) |
| | yl)-7-(2-(ethyl(methyl)amino)ethyl)- | (d, J = 16.6 Hz, 1H), 7.34 (s, 1H), | was replaced with |
| | 1-oxo-3,4-dihydroisoquinolin-2(1H)- | 7.17 (d, J = 5.6 Hz, 1H), 6.82 (d, | (G7) |
| | yl)ethyl)-4-ethoxynicotinonitrile | J = 3.6 Hz, 1H), 6.54-6.21 (m, | Step 2: (6-fluoro- |
| | 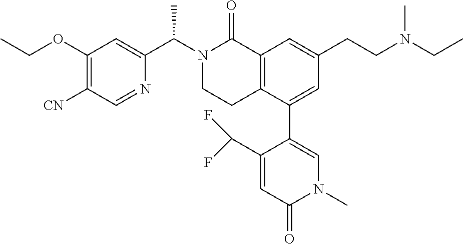 | 1H), 6.03-5.93 (m, 1H), 4.36- 4.26 (m, 2H), 3.61 (d, J = 8.0 Hz, 3H), 3.58-3.38(m, 2H), 3.00- 2.85 (m, 4H), 2.82-2.58 (m, 4H), 2.50 (s, 3H), 1.66 (dd, J = 3.8, 7.0 Hz, 3H), 1.48 (dt, J = 3.2, 7.0 Hz, 3H), 1.18(t, J = 7.2 Hz, 3H) LC-MS: [M + H]⁺ = 564. | 2-methylpyridin-3- yl)boronic acid was replaced with (4- (difluoromethyl)-1- methyl-6-oxo-1,6- dihydropyridin-3- yl)boronic acid |
| 150 | 4-ethoxy-6-(1-(7-(2- | ¹H NMR (400 MHz, Methanol-d₄) | Step 1: |
| | (ethyl(methyl)amino)ethyl)-5-(5- | δ 8.61 (d, J = 2.4 Hz, 1H), 7.99 | intermediate (G1) |
| | hydroxy-2-methylpyridin-3-yl)-1- | (dd, J = 28.2, 2.3 Hz, 2H), 7.30 (s, | Step 2: (6-fluoro- |
| | oxo-3,4-dihydroisoquinolin-2(1H)- | 1H), 7.19 (d, J = 2.6 Hz, 1H), 6.99 | 2-methylpyridin-3- |
| | yl)ethyl)nicotinonitrile | (dd, J = 11.6, 2.8 Hz, 1H), 5.97 | yl)boronic acid |
| | 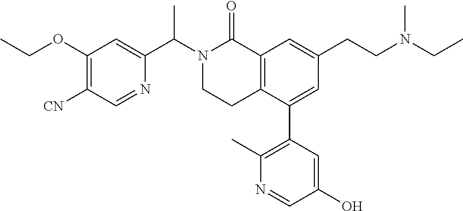 | (dd, J = 7.1, 2.6 Hz, 1H), 4.38- 4.26 (m, 2H), 3.60-3.41 (m, 2H), 3.02 (s, 4H), 2.90 (s, 2H), 2.61 (s, 5H), 2.15 (d, J = 16.8 Hz, 3H), 1.65 (dd, J = 7.2, 1.8 Hz, 3H), 1.48 (d, J = 7.0 Hz, 3H), 1.22 (t, J = 7.3 Hz, 3H). LC-MS: [M + H]⁺ = 514. | was replaced with (5-hydroxy-2- methylpyridin-3- yl)boronic acid |
| 151 | 4-ethoxy-6-(1-(7-(2- | ¹H NMR (400 MHz, Methanol-d₄) | Step 1: |
| | (ethyl(methyl)amino)ethyl)-5-(2- | δ 8.60 (d, J = 1.7 Hz, 1H), 8.52 | intermediate (G1) |
| | ethylpyridin-3-yl)-1-oxo-3,4- | (ddt, J = 2.8, 1.9, 0.9 Hz, 2H), | Step 2: (6-fluoro- |
| | dihydroisoquinolin-2(1H)- | 7.98 (d, J = 1.9 Hz, 1H), 7.57 | 2-methylpyridin-3- |
| | yl)ethyl)nicotinonitrile | (ddd, J = 12.4, 7.7, 1.7 Hz, 1H), | yl)boronic acid |
| | 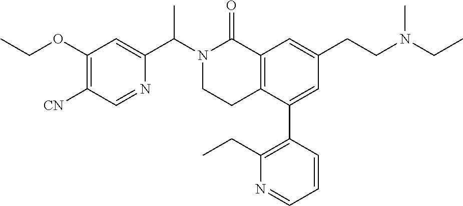 | 7.42-7.28 (m, 2H), 7.19 (d, J = 2.4 Hz, 1H), 5.97 (qd, J = 7.2, 3.2 Hz, 1H), 4.31 (qdd, J = 7.0, 2.0, 1.0 Hz, 2H), 3.63-3.39 (m, 2H), 3.22-2.96 (m, 6H), 2.73 (s, 3H), 2.69-2.44 (m, 4H), 1.65 (d, J = 7.2 Hz, 3H), 1.47 (t, J = 7.0 Hz, 3H), 1.27 (t, J = 7.3 Hz, 3H), 1.09 (dt, J = 10.9, 7.6 Hz, 3H). LC-MS: [M + H]⁺ = 512. | was replaced with (2-ethylpyridin-3- yl)boronic acid |
| 152 | (S)-6-(1-(5-(3-chloropyridin-4-yl)-7- | ¹H NMR (400 MHz, Methanol-d₄) | Step 1: |
| | (2-(ethyl(methyl)amino)ethyl)-1-oxo- | δ 8.69 (d, J = 4.4 Hz, 1H), 8.61 | intermediate (G1) |
| | 3,4-dihydroisoquinolin-2(1H)- | (d, J = 7.3 Hz, 1H), 8.56 (t, J = 5.1 | Step 2: (6-fluoro- |
| | yl)ethyl)-4-ethoxynicotinonitrile | Hz, 1H), 8.01 (d, J = 1.8 Hz, 1H), | 2-methylpyridin-3- |
| | 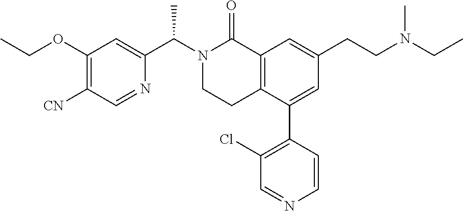 | 7.39 (dd, J = 11.2, 4.8 Hz, 1H), 7.35 (d, J = 1.7 Hz, 1H), 7.19 (d, J = 3.2 Hz, 1H), 5.97 (qd, J = 7.1, 1.9 Hz, 1H), 4.35-4.20 (m, 2H), 3.65-3.37 (m, 2H), 3.05 (s, 4H), 2.93 (d, J = 6.8 Hz, 2H), 2.74- 2.50 (m, 5H), 1.65 (dd, J = 7.1, 4.0 Hz, 3H), 1.47 (td, J = 7.0, 1.1 Hz, 3H), 1.23 (t, J = 7.2 Hz, 3H). LC-MS: [M + H]⁺ = 518. | yl)boronic acid was replaced with (3-chloropyridin-4- yl)boronic acid |
| 153 | 6-(1-(5-(2-chloro-5-(1- | ¹H NMR (400 MHz, Methanol-d₄) | Step 1: |
| | hydroxyethyl)pyridin-3-yl)-7-(2- | δ 8.61 (d, J = 9.1 Hz, 1H), 8.53 (s, | intermediate (G1) |
| | (ethyl(methyl)amino)ethyl)-1-oxo- | 1H), 8.43 (dd, J = 4.4, 2.4 Hz, | Step 2: (6-fluoro- |
| | 3,4-dihydroisoquinolin-2(1H)- | 1H), 7.99 (d, J = 1.9 Hz, 1H), 7.77 | 2-methylpyridin-3- |
| | yl)ethyl)-4-ethoxynicotinonitrile | (dt, J = 13.5, 2.4 Hz, 1H), 7.35 | yl)boronic acid |
| | 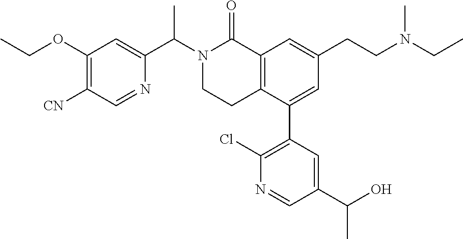 | (dd, J = 4.7, 1.8 Hz, 1H), 7.19 (d, J = 2.2 Hz, 1H), 6.06-5.92 (m, 1H), 4.95 (p, J = 6.3 Hz, 1H), 4.41- 4.22 (m, 2H), 3.66-3.43 (m, 2H), 3.04 (s, 4H), 2.91 (d, J = 7.5 Hz, 2H), 2.81-2.54 (m, 5H), 1.65 (dd, J = 7.2, 4.4 Hz, 3H), 1.48 (td, J = 6.9, 5.6 Hz, 6H), 1.23 (t, J = 7.2 Hz, 3H). LC-MS: [M + H]⁺ = 562. | was replaced with (2-chloro-5-(1- hydroxyethyl) pyridin-3- yl)boronic acid |
| 154 | 4-ethoxy-6-((7-(2- | ¹H NMR (400 MHz, Methanol-d₄) | Step 1: |
| | (ethyl(methyl)amino)ethyl)-5-(5- | δ 8.60 (s, 1H), 8.41 (d, J = 1.7 Hz, | intermediate (G1) |
| | fluoro-2-methylpyridin-4-yl)-1-oxo- | 1H), 8.02 (d, J = 1.8 Hz, 1H), 7.46 | Step 2: (6-fluoro- |
| | 3,4-dihydroisoquinolin-2(1H)- | (d, J = 1.8 Hz, 1H), 7.32 (d, J = | 2-methylpyridin-3- |
| | yl)methyl)nicotinonitrile | 6.0 Hz, 1H), 7.19 (s, 1H), 4.89 (s, | yl)boronic acid |
| | 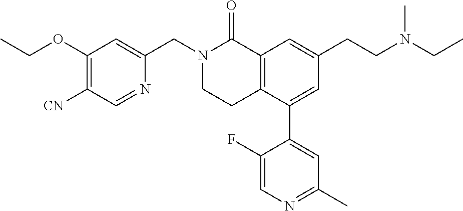 | 2H), 4.31 (q, J = 7.0 Hz, 2H), 3.66 (t, J = 6.6 Hz, 2H), 3.06 (s, 4H), 3.01-2.82 (m, 4H), 2.65 (s, 3H), 2.57 (s, 3H), 1.48 (t, J = 7.0 Hz, 3H), 1.24 (s, 3H). LC-MS: [M + H]⁺ = 502. | was replaced with (2-fluoro-5- methylpyridin-4- yl)boronic acid |
| 155 | 6-((5-(6-amino-2-methylpyridin-3- | ¹H NMR (400 MHz, Methanol-d₄) | Step 1: |
| | yl)-7-(2-(ethyl(methyl)amino)ethyl)- | δ 8.59 (s, 1H), 7.93 (d, J = 1.9 Hz, | intermediate (G1) |
| | 1-oxo-3,4-dihydroisoquinolin-2(1H)- | 1H), 7.34 (d, J = 2.0 Hz, 1H), 7.26 | Step 2: (6-fluoro- |
| | yl)methyl)-4-ethoxynicotinonitrile | (d, J = 8.5 Hz, 1H), 7.18 (s, 1H), | 2-methylpyridin-3- |
| | 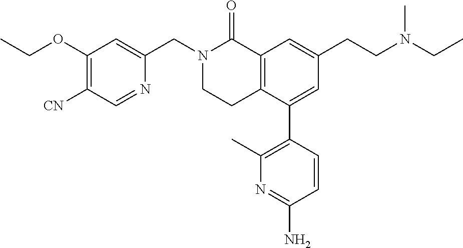 | 6.53 (d, J = 8.4 Hz, 1H), 4.31 (q, J = 7.0 Hz, 2H), 3.65 (t, J = 6.6 Hz, 2H), 3.38-3.34 (m, 1H), 3.23 (q, J = 7.3 Hz, 3H), 3.16-3.04 (m, 2H), 2.88 (s, 3H), 2.81-2.69 (m, 2H), 2.52 (s, 1H), 2.11 (s, 3H), 1.47 (t, J = 7.0 Hz, 3H), 1.34 (t, J = 7.3 Hz, 4H). LC-MS: [M + H]⁺ = 499. | yl)boronic acid was replaced with (6-amino-2- methylpyridin-3- yl)boronic acid |
| 156 | (R)-6-(1-(5-(2,5-dimethylpyridin-3- | ¹H NMR (400 MHz, Methanol-d₄) | Step 1: |
| | yl)-7-(2-(ethyl(methyl)amino)ethyl)- | δ 8.60 (d, J = 2.9 Hz, 1H), 8.29 | intermediate (G1) |
| | 1-oxo-3,4-dihydroisoquinolin-2(1H)- | (d, J = 2.1 Hz, 1H), 7.93 (d, J = | Step 2: (6-fluoro- |
| | yl)ethyl)-4-ethoxynicotinonitrile | 1.9 Hz, 1H), 7.42 (dd, J = 12.7, | 2-methylpyridin-3- |
| | 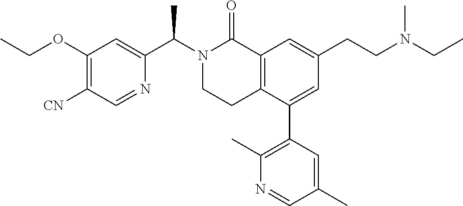 | 2.1 Hz, 1H), 7.22 (dd, J = 27.4, 2.6 Hz, 2H), 5.98 (qd, J = 7.1, 3.3 Hz, 1H), 4.31 (qd, J = 7.1, 2.3 Hz, 2H), 3.61-3.38 (m, 2H), 2.96- 2.85 (m, 2H), 2.78-2.68 (m, 2H), 2.67-2.48 (m, 4H), 2.35 (d, J = 4.9 Hz, 6H), 2.23 (d, J = 17.6 Hz, 3H), 1.64 (dd, J = 7.1, 2.3 Hz, 3H), 1.47 (t, J = 7.0 Hz, 3H), 1.11 (t, J = 7.2 Hz, 3H). LC-MS: [M + H]⁺ = 512. | yl)boronic acid was replaced with (2,5- dimethylpyridin-3- yl)boronic acid |
| 157 | (S)-4-ethoxy-6-(1-(7-(2- | ¹H NMR (400 MHz, Methanol-d₄) | Step 1: |
| | (ethyl(methyl)amino)ethyl)-5-(1- | δ 8.63 (s, 1H), 7.98 (d, J = 1.9 Hz, | intermediate (G1) |
| | methyl-1H-imidazol-5-yl)-1-oxo-3,4- | 1H), 7.76 (s, 1H), 7.40 (d, J = 1.9 | Step 2: (6-fluoro- |
| | dihydroisoquinolin-2(1H)- | Hz, 1H), 7.21 (s, 1H), 6.99-6.92 | 2-methylpyridin-3- |
| | yl)ethyl)nicotinonitrile | (m, 1H), 6.00 (q, J = 7.1 Hz, 1H), | yl)boronic acid |
| | 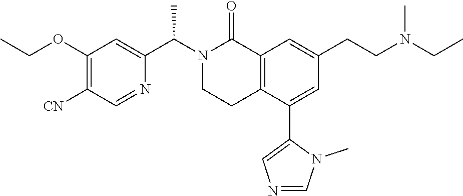 | 4.33 (q, J = 7.0 Hz, 2H), 3.64- 3.44 (m, 5H), 2.92 (dd, J = 10.2, 6.0 Hz, 2H), 2.84-2.69 (m, 4H), 2.60 (q, J = 7.2 Hz, 2H), 2.37 (s, 3H), 1.67 (d, J = 7.1 Hz, 3H), 1.49 (t, J = 7.0 Hz, 3H), 1.13 (t, J = 7.2 Hz, 3H). LC-MS: [M + H]⁺ = 487. | was replaced with v(1-methyl-1H- imidazol-5- yl)boronic acid |
| 158 | 6-(1-(5-cyclopropyl-7-(2- | ¹H NMR (400 MHz, Methanol-d₄) | cyclopropyl- |
| | (ethyl(methyl)amino)ethyl)-1-oxo- | δ 8.64 (s, 1H), 7.72 (s, 1H), 7.19 | boronic acid |
| | 3,4-dihydroisoquinolin-2(1H)- | (s, 1H), 7.17 (d, J = 7.1 Hz, 1H), | |
| | yl)ethyl)-4-ethoxynicotinonitrile | 5.97 (q, J = 7.0 Hz, 1H), 4.74- | |
| | 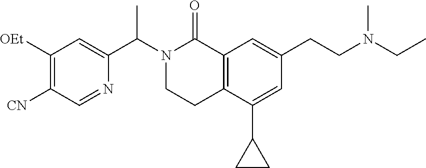 | 4.54 (m, 2H), 4.31 (q, J = 7.0 Hz, 2H), 3.75-3.45 (m, 2H), 3.26- 2.91 (m, 8H), 2.90-2.71 (m, 2H), 2.08-1.82 (m, 2H), 1.67 (d, J = 7.1 Hz, 3H), 1.47 (t, J = 7.0 Hz, 4H), 1.36-1.24 (m, 4H), 1.09- 0.94 (m, 2H), 0.78-0.60 (m, 2H). LC-MS: [M + H]⁺ = 447. | |
| 159 | 4-ethoxy-6-(1-(7-(2- | ¹H NMR (400 MHz, Methanol-d₄) | Step 1: |
| | (ethyl(methyl)amino)ethyl)-1-oxo-5- | δ 8.62 (s, 1H), 8.42 (s, 1H), 7.88 | intermediate (G1) |
| | (1,2,3,6-tetrahydropyridin-4-yl)-3,4- | (d, J = 1.8 Hz, 1H), 7.35 (d, J = | Step 2: (6-fluoro- |
| | dihydroisoquinolin-2(1H)- | 1.9 Hz, 1H), 7.19 (s, 1H), 5.96 (q, | 2-methylpyridin-3- |
| | yl)ethyl)nicotinonitrile | J = 7.1 Hz, 1H), 5.68 (p, J = 1.8 | yl)boronic acid |
| | 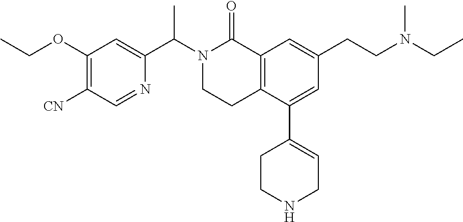 | Hz, 1H), 4.31 (qd, J = 7.0,1.7 Hz, 2H), 3.83 (q, J = 2.6 Hz, 2H), 3.64- 3.43 (m, 4H), 3.42-3.34 (m, 2H), 3.26 (t, J = 7.3 Hz, 2H), 3.16- 3.06 (m, 2H), 3.03-2.83 (m, 5H), 2.68-2.53 (m, 2H), 1.66 (d, J = 7.1 Hz, 3H), 1.47 (t, J = 7.0 Hz, 3H), 1.36 (t, J = 7.3 Hz, 3H). LC-MS: [M + H]⁺ = 488. | was replaced with (1,2,3,6- tetrahydropyridin- 4-yl)boronic acid |
| 160 | (S)-6-(1-(5-(5-cyclopropyl-2- | ¹H NMR (400 MHz, Methanol-d₄) | Step 1: |
| | methylpyridin-3-yl)-7-(2- | δ 8.61 (s, 1H), 8.26 (d, J = 2.3 Hz, | intermediate (G1) |
| | (ethyl(methyl)amino)ethyl)-1-oxo- | 1H), 7.93 (d, J = 1.8 Hz, 1H), 7.28- | Step 2: (6-fluoro- |
| | 3,4-dihydroisoquinolin-2(1H)- | 7.20 (m, 2H), 7.18 (d, J = 2.9 | 2-methylpyridin-3- |
| | yl)ethyl)-4-ethoxynicotinonitrile | Hz, 1H), 5.98 (dd, J = 7.1, 3.4 Hz, | yl)boronic acid |
| | 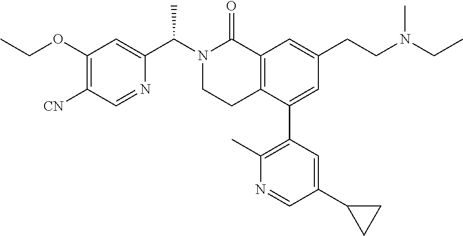 | 1H), 4.31 (q, J = 7.0 Hz, 2H), 3.53 (s, 1H), 3.50-3.37 (m, 1H), 2.92 (dd, J = 10.3, 5.7 Hz, 2H), 2.79 (d, J = 9.1 Hz, 2H), 2.69-2.61 (m, 2H), 2.61-2.50 (m, 2H), 2.40 (s, 3H), 2.22 (d, J = 17.2 Hz, 3H), 2.01-1.94 (m, 1H), 1.64 (dd, J = 7.1, 0.9 Hz, 3H), 1.47 (td, J = 7.0, 1.1 Hz, 3H), 1.13 (t, J = 7.2 Hz, 3H), 1.04 (ddd, J = 8.4, 4.2, 1.9 Hz, 2H), 0.74 (ddd, J = 8.1, 4.4, 1.8 Hz, 2H). LC-MS: [M + H]⁺ = 538 | was replaced with (5-cyclopropyl-2- methylpyridin-3- yl)boronic acid |

### Example 161: (S)-6-(1-(5-(1,3-dimethyl-2-oxo-1,2-dihydropyridin-4-yl)-7-(2-(ethyl(methyl)amino)ethyl)-1-oxo-3,4-dihydroisoquinolin-2(1H)-yl)ethyl)-4-ethoxynicotinonitrile (161)

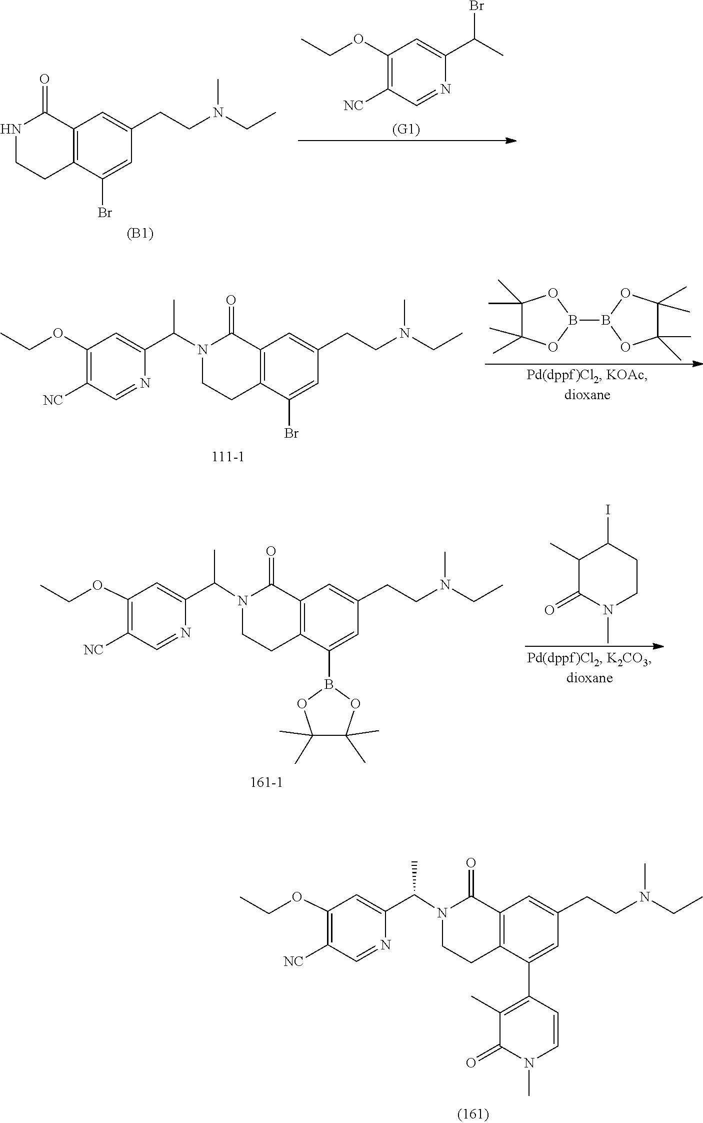

Step 1: Compound 111-1 was obtained using the method described in step 1 of Example 111.

Step 2: To a solution of compound 111-1 (500 mg, 1.03 mmol, 1.0 eq) and compound 4,4,4′,4′,5,5,5′,5′-octamethyl-2,2′-bi(1,3,2-dioxaborolane) (312 mg, 1.24 mg, 0.742 mmol, 1.2 eq) in dioxane (5 mL) was added KOAc (252 mg, 2.58 mmol, 2.5 eq) and Pd(dppf)Cl₂ (84 mg, 0.103 mmol, 0.1 eq). The mixture was stirred at 100° C. for 2 hrs under N₂ atmosphere. The mixture was diluted with H₂O (30 mL) and extracted with ethyl acetate (20 mL×3). The combined organic layers were washed with brine (15 mL), dried over anhydrous Na₂SO₄, filtered and concentrated to give 4-ethoxy-6-(1-(7-(2-(ethyl(methyl)amino)ethyl)-1-oxo-5-(4,4,5,5-tetramethyl-1,3,2-dioxaborolan-2-yl)-3,4-dihydroisoquinolin-2(1H)-yl)ethyl)nicotinonitrile (161-1) (600 mg, 47.8% purity, crude) as oil, which was used directly in the next step.

Step 3: To a solution of compound 161-1 (600 mg, 47.8% purity, 0.539 mmol, 1.0 eq) and compound 4-iodo-1,3-dimethylpyridin-2(1H)-one (161 mg, 0.646 mmol, 1.2 eq) in dioxane/H₂O (6 mL/1.2 mL) was added K₂CO₃ (223 mg, 1.616 mmol, 3.0 eq) and Pd(dppf)Cl₂ (44 mg, 0.0539 mmol, 0.1 eq). The mixture was stirred at 80° C. for 3 hrs under N₂ atmosphere. The mixture was cooled to 25° C. and diluted with acetonitrile (10 mL). Thiourea (resin) (400 mg) was added and the resulting mixture was stirred at room temperature overnight. The mixture was filtered and concentrated. The residue was purified by Prep-HPLC (Column: Phenomenex synergi C18 250*25*10 um; Condition: A=water (0.1% TFA)-ACN; 12˜40% of B, B=acetonitrile, flow rate: 25 ml/min) to give compound racemate (170 mg) as oil. Racemate (170 mg) was separated by SFC (column: Phenomenex-Cellulose-2 (250 mm*30 mm, 10 um), Condition: 0.1% NH₄OH MeOH Flow Rate: 70 g/ml) to give (S)-6-(1-(5-(1,3-dimethyl-2-oxo-1,2-dihydropyridin-4-yl)-7-(2-(ethyl(methyl)amino)ethyl)-1-oxo-3,4-dihydroisoquinolin-2(1H)-yl)ethyl)-4-ethoxynicotinonitrile (161) as a brown solid. ¹H NMR (400 MHz, Methanol-d₄) δ 8.61 (d, J=3.9 Hz, 1H), 7.93 (d, J=1.4 Hz, 1H), 7.55 (t, J=6.4 Hz, 1H), 7.23 (t, J=1.8 Hz, 1H), 7.18 (s, 1H), 6.18 (dd, J=6.9, 12.9 Hz, 1H), 5.98 (dq, J=2.1, 7.0 Hz, 1H), 4.31 (q, J=7.0 Hz, 2H), 3.62 (s, 3H), 3.56 (ddd, J=5.6, 7.9, 13.0 Hz, 1H), 3.50-3.42 (m, 1H), 2.95-2.89 (m, 2H), 2.79-2.73 (m, 2H), 2.72-2.59 (m, 4H), 2.39 (s, 3H), 1.85 (d, J=17.8 Hz, 3H), 1.65 (dd, J=2.4, 7.1 Hz, 3H), 1.47 (t, J=7.0 Hz, 3H), 1.13 (t, J=7.3 Hz, 3H). LC-MS: [M+H]⁺=528

### Example 169: (S)-4-ethoxy-6-(1-(7-(2-(ethyl(methyl)amino)ethyl)-5-(5-fluoro-1,4-dimethyl-6-oxo-1,6-dihydropyridin-3-yl)-1-oxo-3,4-dihydroisoquinolin-2(1H)-yl)ethyl)nicotinonitrile (169)

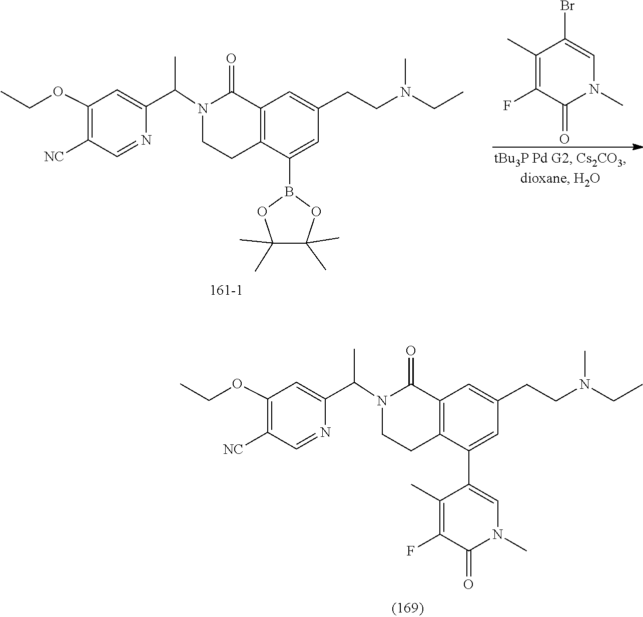

A solution of compound 161-1 (600 mg, 50% purity, 0.563 mmol, 1.0 eq), compound 5-bromo-3-fluoro-1,4-dimethylpyridin-2(1H)-one (186 mg, 0.845 mmol, 1.2 eq), Cs₂CO₃ (551 mg, 1.69 mmol, 3.0 eq) and t-Bu₃P Pd G2 (29 mg, 0.0563 mmol, 0.1 eq, cas: 1375325-71-5) in dioxane/water (8/2 mL) was stirred at 80° C. for 2 hrs under N₂ atmosphere. The reaction solution was diluted with acetonitrile (20 mL) and added thiourea resin (300 mg). The resulting mixture was stirred at 25° C. for 16 hrs. The mixture was filtered and concentrated. The residue was purified by Prep-HPLC (Column: Waters Xbridge 150*50 10 u; Condition: A=water (0.05% ammonia hydroxide)-acetonitrile; gradient: 28-58% of acetonitrile, flow rate: 60 ml/min) to give racemate compound (110 mg) as oil. The racemate was purified by Prep-SFC (column: DAICEL CHIRALCEL IC (250 mm*30 mm, 10 um); Gradient Time: 2.7 min; 60 min; Condition: 0.1% NH₃H₂O MEOH; Flow Rate: 80 g/min; 65% of B) to give compound 169 as off-white solid. ¹H NMR (400 MHz, Methanol-d₄) δ ppm 8.62 (d, J=3.2 Hz, 1H), 7.93 (d, J=1.6 Hz, 1H), 7.35 (d, J=14.4 Hz, 1H), 7.28 (d, J=1.2 Hz, 1H), 7.19 (d, J=3.6 Hz, 1H), 6.04-5.91 (m, 1H), 4.36-4.25 (m, 2H), 3.62 (d, J=6.8 Hz, 3H), 3.56 (dt, J=4.8, 8.4 Hz, 1H), 3.52-3.43 (m, 1H), 2.91-2.84 (m, 2H), 2.81-2.62 (m, 4H), 2.55 (q, J=7.2 Hz, 2H), 2.32 (s, 3H), 1.93 (dd, J=2.8, 17.6 Hz, 3H), 1.65 (dd, J=2.2, 7.2 Hz, 3H), 1.47 (t, J=7.0 Hz, 3H), 1.10 (t, J=7.2 Hz, 3H). LC-MS: [M+H]⁺=546

### Example 173: (S)-6-(1-(5-(3,6-dimethylpyridazin-4-yl)-7-(2-(ethyl(methyl)amino)ethyl)-1-oxo-3,4-dihydroisoquinolin-2(1H)-yl)ethyl)-4-ethoxynicotinonitrile (173)

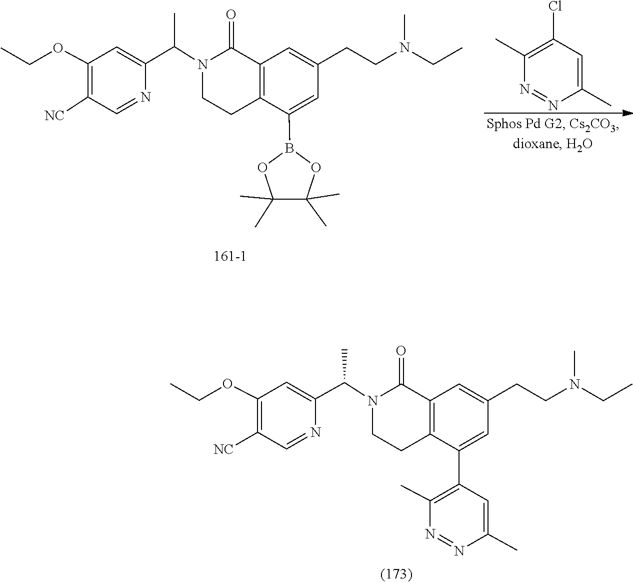

The mixture of 161 (1460 mg, 2.74 mmol), 4-chloro-3,6-dimethylpyridazine (489 mg, 3.43 mmol), Cs₂CO₃ (938 mg, 2.88 mmol) and SPhos Pd G2 (198 mg, 0.274 mmol) in Dioxane (Volume: 75 mL, Ratio: 10.00)/H2O (Volume: 7.5 mL, Ratio: 1.000) was stirred for 20 hr at 90° C. under N2 atmosphere. The mixture was concentrated under reduced pressure and the residue was purified by silica (DCM/MeOH=10/1) to afford crude product as brown solid, which was purified by pre-HPLC (Column: Phenomenex synergi C18 250*25*10 um; Condition: A=water (0.1% TFA)-ACN; 12˜40% of B, B=acetonitrile, flow rate: 25 ml/min) to afford the racemate compound (810 mg, 1.450 mmol, 52.9% yield) as pale yellow solid. Racemate (810 mg) was separated by SFC (column: Phenomenex-Cellulose-2 (250 mm*30 mm, 10 um), Condition: 0.1% NH₄OH MeOH Flow Rate: 70 g/ml) to give: (S)-6-(1-(5-(3,6-dimethylpyridazin-4-yl)-7-(2-(ethyl(methyl)amino)ethyl)-1-oxo-3,4-dihydroisoquinolin-2(1H)-yl)ethyl)-4-ethoxynicotinonitrile 173 (300 mg, 21% yield) as yellow solid. ¹H NMR (400 MHz, Methanol-d₄) δ ppm 8.61 (d, J=2.3 Hz, 1H), 7.98 (d, J=1.9 Hz, 1H), 7.41 (d, J=12.2 Hz, 1H), 7.29 (d, J=1.9 Hz, 1H), 7.19 (d, J=3.0 Hz, 1H), 6.03-5.92 (m, 1H), 4.31 (qd, J=7.0, 2.9 Hz, 2H), 3.60-3.41 (m, 2H), 2.90 (dd, J=10.0, 6.2 Hz, 2H), 2.74-2.47 (m, 9H), 2.41 (d, J=17.0 Hz, 3H), 2.34 (s, 3H), 1.65 (dd, J=7.1, 1.4 Hz, 3H), 1.47 (t, J=7.0 Hz, 3H), 1.10 (t, J=7.2 Hz, 3H). LC-MS: [M+H]⁺=513

### Example 196: 4-ethoxy-6-((7-(2-(ethyl(methyl)amino)ethyl)-5-(1-methyl-6-oxo-4-(trifluoromethyl)-1,6-dihydropyridin-3-yl)-1-oxo-3,4-dihydroisoquinolin-2(1H)-yl)methyl)nicotinonitrile (196)

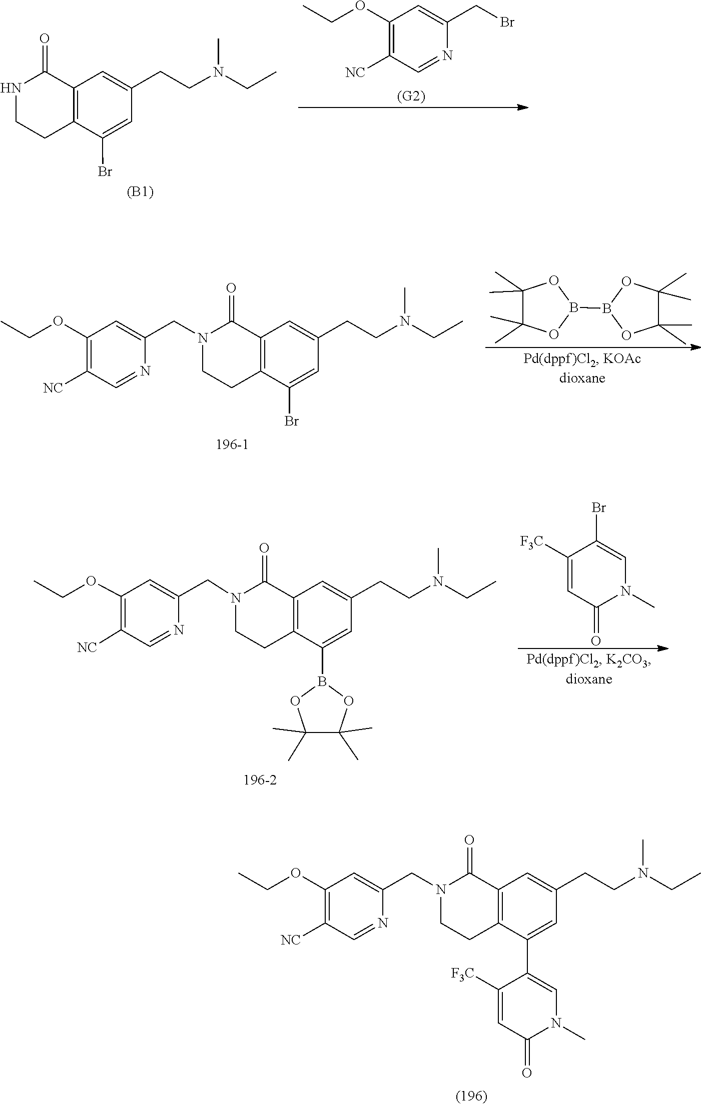

Step 1: Compound 196-1 was obtained using the method described in step 1 of Example 111.

Step 2: Compound 196-2 was obtained using the method described in step 2 of Example 161.

Step 3: To a solution of compound 196-2 (300 mg, 0.32 mmol, 1 eq, 70% purity) in dioxane (5 mL) and H₂O (1 mL) was added 5-bromo-1-methyl-4-(trifluoromethyl)pyridin-2(1H)-one (98 mg, 0.38 mmol, 1 equiv), Cs₂CO₃ (311 mg, 0.96 mmol, 3 equiv), tBu₃ Pd G2 (16 mg, 0.032 mmol, 0.1 equiv) at 25° C. under nitrogen atmosphere. The reaction mixture was stirred at 80° C. for 3 hours. MeCN (10 mL) was added. The resulting mixture was stirred with thiourea resin (1 g) at 25° C. for 12 hrs. The resulting mixture was filtered and washed with MeCN (20 mL×3). The combined organic phase was concentrated under reduced pressure. The residue was purified by Prep-HPLC (Waters Xbridge 150*25*5, Gradient: 19%-49% of B. A: water (10 mM NH4HCO3), B: Acetonitrile, flowrate: 25 mL/min) to afford 4-ethoxy-6-((7-(2-(ethyl (methyl)amino)ethyl)-5-(1-methyl-6-oxo-4-(trifluoromethyl)-1,6-dihydropyridin-3-yl)-1-oxo-3,4-dihydroisoquinolin-2(1H)-yl)methyl)nicotinonitrile 196 (33.65 mg) as white solid. ¹H NMR (400 MHz, Methanol-d₄) δ 8.62 (s, 1H), 7.96 (d, J=1.6 Hz, 1H), 7.73 (s, 1H), 7.35 (s, 1H), 7.19 (s, 1H), 6.97 (s, 1H), 4.95 (s, 1H), 4.88 (brs, 1H), 4.32 (q, J=7.0 Hz, 2H), 3.71-3.62 (m, 5H), 2.99-2.86 (m, 3H), 2.77-2.65 (m, 3H), 2.60-2.52 (m, 2H), 2.34 (d, J=1.2 Hz, 3H), 1.49 (t, J=7.0 Hz, 3H), 1.12 (t, J=7.2 Hz, 3H).

LC-MS: [M+H]⁺=568.

Following a similar procedure to that described in Example 161 (unless otherwise specified). The following compounds were prepared from their corresponding precursors.

| Ex. | Name/Structure | ¹HNMR/LC-MS | Precursors |
|---|---|---|---|
| 162 | 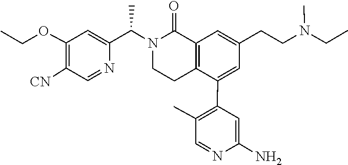 (S)-6-(1-(5-(2-amino-5-methylpyridin-4-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ ppm 8.61 (s, 1H), 7.90 (d, J = 1.6 Hz, 1H), 7.82 (d, J = 4.6 Hz, 1H), 7.20 (dm, 2H), 6.36 (d,J = 12.8 Hz, 1H), 5.98 (m, 1H), 4.31 (q, J = 7.0 Hz, 2H), 3.61 - 3.39 (m, 2H), 2.94 - 2.85 (m, 2H), 2.75 - 2.53 (m, 6H), 2.36 (s,3H), 1.87 (d, J = 17.6 Hz, 3H), 1.65 (d, J = 7.2 Hz, 3H), 1.47 (t, J = 7.0 Hz, 3H), 1.12 (t, J = 7.2 Hz, 3H). LC-MS: [M + H]⁺ = 513.3 | Step 1: intermediate (G1) Step 3: 4-iodo-1,3- dimethylpyridin- 2(1H)-one was replaced with 4- bromo-5- methylpyridin-2- amine |
| 163 | 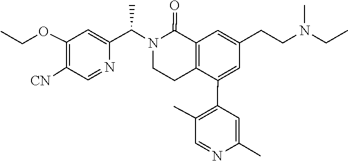 (S)-6-(1-(5-(2,5-dimethylpyridin-4- yl)-7-(2-(ethyl(methyl)amino)ethyl)- 1-oxo-3,4-dihydroisoquinolin-2(1H)- yl)ethyl)-4-ethoxynicotinonitrile | ¹H NMR (400 MHz, Methanol-d₄) δ ppm 8.60 (s, 1H), 8.51 (br s, 1H), 8.35 (d, J = 4.4 Hz, 1H), 7.99 (d, J = 1.6 Hz, 1H), 7.31 (d,J = 2.0 Hz, 1H), 7.19 (d, J = 3.2 Hz, 1H), 7.09 (d, J = 12.0 Hz, 1H), 5.97 (m, 1H), 4.34 (q, J = 6.8 Hz, 2H), 3.62 - 3.40 (m, 2H), 3.28(s, 1H), 3.23- 3.08 (m, 4H), 2.84 (s, 3H), 2.73 - 2.53 (m, 2H), 2.52 (d, J = 5.2 Hz, 3H), 2.05 (d, J = 16.6 Hz, 3H), 1.66 (d, J = 7.2Hz, 3H), 1.48 (t, J = 7.0 Hz, 3H), 1.32 (t, J = 7.2 Hz, 3H), 1.20 (s, 1H). LC-MS: [M + H]⁺ = 512.3 | Step 1: intermediate (G1) Step 3: 4-iodo-1,3- dimethylpyridin- 2(1H)-one was replaced with 4- bromo-2,5- dimethylpyridine |
| 164 | 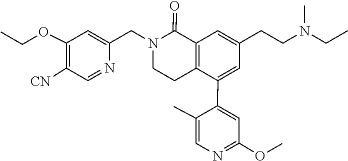 4-ethoxy-6-((7-(2- (ethyl(methyl)amino)ethyl)-5-(2- methoxy-5-methylpyridin-4-yl)-1- oxo-3,4-dihydroisoquinolin-2(1H)- yl)methyl)nicotinonitrile | ¹H NMR (400 MHz, Methanol-d₄) δ ppm 8.59 (s, 1H), 8.05 (t, J = 0.8 Hz, 1H), 7.95 (d, J = 1.9 Hz, 1H), 7.27 (d, J = 1.9 Hz, 1H), 7.18 (s, 1H), 6.61 (s, 1H), 4.88 (d, J = 2.4 Hz, 2H), 4.30 (q, J = 7.0 Hz, 2H), 3.90 (s, 3H), 3.70 - 3.59 (m, 2H), 3.05 - 2.91 (m, J = 4.1 Hz, 4H), 2.86 - 2.78 (m, 2H), 2.77 (s, 2H), 2.54 (s, 3H), 1.98 (d, J = 0.8 Hz, 3H), 1.47 (t, J = 7.0 Hz, 3H), 1.19 (t, J = 7.2 Hz, 3H). LC-MS: [M + H]⁺ = 514.3 | Step 1: intermediate (G1) was replaced with intermediate (G2) Step 3: 4-iodo-1,3- dimethylpyridin- 2(1H)-one was replaced with 4- bromo-2-methoxy- 5-methylpyridine |
| 165 | 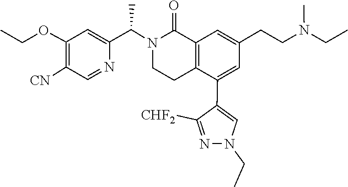 (S)-6-(1-(5-(3-(difluoromethyl)-1- ethyl-1H-pyrazol-4-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d4) δ ppm 8.62 (s, 1H), 7.88 (d, J = 1.6 Hz, 1H), 7.73 (s, 1H), 7.36 (d, J = 1.6 Hz, 1H), 7.18 (s,1H), 6.83- 6.51 (m, 1H), 5.98 (q, J = 7.2 Hz, 1H), 4.35 - 4.21 (m, 4H), 3.58- 3.49 (m, 1H), 3.48 - 3.40 (m, 1H), 2.92 - 2.85 (m,2H), 2.82 - 2.69 (m, 4H), 2.61 (q, J = 7.2 Hz, 2H), 2.37 (s, 3H), 1.65 (d, J = 7.2 Hz, 3H), 1.53- 1.45 (m, 6H), 1.13 (t, J = 7.2 Hz, 3H). LC-MS: [M + H]⁺ = 551.4 | Step 1: intermediate (G1) Step 3: 4-iodo-1,3- dimethylpyridin- 2(1H)-one was replaced with 4- bromo-3- (difluoromethyl)-1- ethyl-1H-pyrazole |
| 166 | 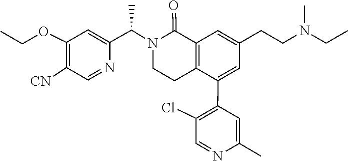 (S)-6-(1-(5-(5-chloro-2- methylpyridin-4-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ ppm 8.61 (d, J = 6.9 Hz, 1H), 8.52 (d, J = 4.6 Hz, 1H), 7.97 (d, J = 1.9 Hz, 1H), 7.32 -7.24 (m, 2H), 7.18 (d, J = 3.2 Hz, 1H), 6.04 - 5.93 (m, 1H), 4.31 (qd, J = 7.1,2.5 Hz, 2H), 3.63- 3.41 (m, 2H), 2.97 -2.85 (m, 2H), 2.76 - 2.52 (m, 9H), 2.35 (s, 3H), 1.65 (dd, J = 7.2, 4.1 Hz, 3H), 1.47 (t, J = 7.0 Hz, 3H), 1.11 (t, J = 7.2 Hz, 3H). LC-MS: [M + H]⁺ = 532.2 | Step 1: intermediate (G1) Step 3: 4-iodo-1,3- dimethylpyridin- 2(1H)-one was replaced with 4- bromo-5-chloro-2- methylpyridine |
| 167 | 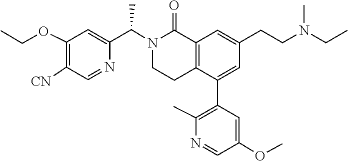 (S)-4-ethoxy-6-(1-(7-(2- (ethyl(methyl)amino)ethyl)-5-(5- methoxy-2-methylpyridin-3-yl)-1- oxo-3,4-dihydroisoquinolin-2(1H)- yl)ethyl)nicotinonitrile | ¹H NMR (400 MHz, Methanol-d₄) δ ppm 8.63 (d, J = 2.6 Hz, 1H), 8.57 (s, 1H), 8.17 (d, J = 2.6 Hz, 1H),7.96 (d, J = 1.8 Hz, 1H),7.48 (d, J = 7.6 Hz, 1H), 7.39-7.35 (m, 1H), 7.29 (d, J = 1.3 Hz, 1H), 7.26- 7.17 (m, 2H), 6.00 (m, 1H), 4.33 (m, 2H), 3.94-3.85 (m, 3H), 3.63- 3.53 (m, 1H), 3.52-3.42 (m, 1H), 2.96-2.87 (m,2H), 2.76-2.68 (m, 2H), 2.68-2.61 (m, 1H), 2.61-2.51 (m, 2H), 2.68-2.51 (m, 1H), 2.35 (s, 2H), 2.39 (s,1H), 2.26-2.17 (m, 4H), 1.67 (t, J = 7.2 Hz,3H), 1.49 (t, J = 7.0 Hz, 3H), 1.13 (t, J = 7.2 Hz, 3H). LC-MS: [M + H]⁺ = 528.3 | Step 1: intermediate (G1) Step 3: 4-iodo-1,3- dimethylpyridin- 2(1H)-one was replaced with 3- bromo-5-methoxy- 2-methylpyridine |
| 168 | 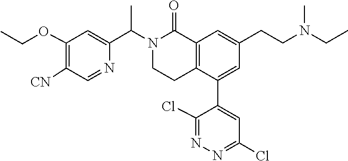 6-(1-(5-(3,6-dichloropyridazin-4-yl)- 7-(2-(ethyl(methyl)amino)ethyl)-1- oxo-3,4-dihydroisoquinolin-2(1H)- yl)ethyl)-4-ethoxynicotinonitrile | ¹H NMR (400 MHz, Methanol-d₄) δ ppm 8.61 (d, J = 8.6 Hz, 1H), 8.07 (d, J = 1.9 Hz, 1H), 7.92 (d, J = 10.0 Hz, 1H), 7.43(d, J = 1.9 Hz, 1H), 7.20 (d, J = 4.3 Hz, 1H), 5.97 (qd, J = 7.1, 3.1 Hz, 1H), 4.39 - 4.22 (m, 2H), 3.66 - 3.46 (m, 2H), 3.23 (d, J = 8.9 Hz, 2H), 3.17 - 3.03 (m, 4H), 2.85 - 2.61 (m, 5H), 1.66 (dd, J = 7.2, 5.1 Hz, 3H), 1.48 (td, J = 7.0, 1.6 Hz, 3H), 1.30 (t, J = 7.3 Hz, 3H). LC-MS: [M + H]⁺ = 553.2 | Step 1: intermediate (G1) Step 3: 4-iodo-1,3- dimethylpyridin- 2(1H)-one was replaced with 4- bromo-3,6- dichloropyridazine |
| 170 | 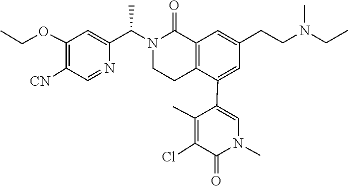 (S)-6-(1-(5-(5-chloro-1,4-dimethyl-6- oxo-1,6-dihydropyridin-3-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ ppm 8.61 (d, J = 4.8 Hz, 1H), 7.93 (d, J = 1.9 Hz, 1H), 7.49 (d, J = 14.5 Hz, 1H), 7.28 (t, J = 1.6 Hz, 1H), 7.18 (d, J = 4.2 Hz, 1H), 5.98 (qd, J = 7.1, 3.8 Hz, 1H), 4.39 - 4.25 (m, 2H), 3.62 (d, J = 6.7 Hz, 3H), 3.60 - 3.53 (m, 1H), 3.53 - 3.40 (m, 1H), 2.96 - 2.80 2H), 2.68 (qd, J = 9.4, 8.0, 4.7 Hz, 4H), 2.55 (q, J = 7.2 Hz, 2H), 2.33 (s, 3H), 2.06 (d, J = 18.2 Hz, 3H), 1.65 (dd, J = 7.1, 3.5 Hz, 3H), 1.53- 1.42(m, 3H), 1.10(t, J = 7.2 Hz, 3H). LC-MS: [M + H]⁺ = 562 | Step 1: intermediate (G1) Step 3: 4-iodo-1,3- dimethylpyridin- 2(1H)-one was replaced with 5- bromo-3-chloro-1,4- dimethylpyridin- 2(1H)-one |
| 171 | 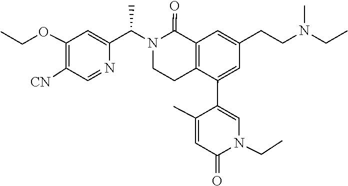 (S)-4-ethoxy-6-(1-(7-(2- (ethyl(methyl)amino)ethyl)-5-(1- ethyl-4-methyl-6-oxo-1,6- dihydropyridin-3-yl)-1-oxo-3,4- dihydroisoquinolin-2(1H)- yl)ethyl)nicotinonitrile | ¹H NMR (400 MHz, Methanol-d₄) δ ppm 8.54-8.49 (m, 1H), 7.82 (s, 1H), 7.37 (d, J = 14.1 Hz, 1H), 7.26 (s, 1H), 7.18 (d, J = 1.6Hz, 1H), 7.09 (s, 1H), 7.06 (s, 1H), 6.74 (s, 1H), 6.40 (d, J = 4.8 Hz, 1H), 5.93- 5.84 (m, 1H), 4.21 (q, J = 6.9 Hz, 2H), 3.99-3.87(m, 2H), 3.53-3.30 (m, 2H), 2.86-2.76 (m, 2H), 2.74- 2.59(m, 4H), 2.56-2.47 (m, 3H), 2.27 (s, 3H), 1.85 (d, J = 17.0 Hz,3H), 1.55 (d, J = 7.1 Hz, 3H), 1.41-1.34 (m, 3H), 1.27- 1.17 (m, 5H), 1.06-0.99 (m, 3H). LC-MS: [M + H]⁺ = 542 | Step 1: intermediate (G1) Step 3: 4-iodo-1,3- dimethylpyridin- 2(1H)-one was replaced with 5- bromo-1-ethyl-4- methylpyridin- 2(1H)-one |
| 172 | 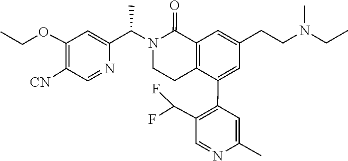 (S)-6-(1-(5-(5-(difluoromethyl)-2- methylpyridin-4-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ ppm 8.83 (d, J = 6.0 Hz, 1H), 8.58 (s, 1H), 8.05 (br s, 1H), 7.14(s,1H), 7.05 - 6.92 (m,2H), 6.56 - 6.19 (m, 1H), 6.08 (q, J = 7.1 Hz, 1H), 4.22 (dq, J = 3.3, 7.0 Hz, 2H), 3.58 - 3.29 (m, 2H), 2.94 - 2.84 (m, 2H), 2.77 -2.69 (m, 2H), 2.64 (d, J = 10.0 Hz, 3H), 2.60 - 2.43 (m, 4H), 2.36 (s, 3H), 1.61 (dd, J = 3.4, 7.0 Hz, 3H), 1.49 (dt, J = 2.8, 6.9 Hz, 3H), 1.11 (t, J = 7.2 Hz, 3H). LC-MS: [M + H]⁺ = 548 | Step 1: intermediate (G1) Step 3: 4-iodo-1,3- dimethylpyridin- 2(1H)-one was replaced with 4- bromo-5- (difluoromethyl)-2- methylpyridine |
| 174 | 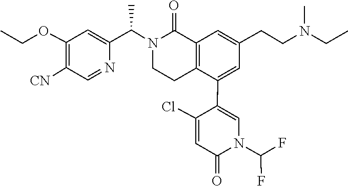 (S)-6-(1-(5-(4-chloro-1- (difluoromethyl)-6-oxo-1,6- dihydropyridin-3-yl)-7-(2- (ethyl(methyl)amino)ethyl)-1-oxo- 3,4-dihydroisoquinolin-2(1H)- yl)ethyl)-4 ethoxynicotinonitrile | ¹H NMR (400 MHz, Methanol-d₄) δ ppm 8.52 (d, J = 6.1 Hz, 1H), 7.98 (s, 1H), 7.65-7.37 (m, 1H), 7.75(brd, J = 5.7 Hz, 1H),7.33-7.24 (m, 1H), 7.10 (s, 1H), 6.93 (brd, J = 6.5 Hz, 1H), 6.69 (d, J = 8.6 Hz, 1H), 6.74-6.62 (m, 1H), 6.01 (brd, J = 6.2 Hz,1H), 4.20-4.09 (m, 2H), 3.55-3.33 (m, 2H), 2.82-2.69 (m, 2H),2.69-2.48 (m, 4H), 2.42 (q, J = 7.2 Hz, 2H), 2.23 (s, 3H),1.61- 1.55 (m, 3H), 1.42 (brt, J = 6.7 Hz, 2H), 1.47-1.34(m, 1H), 1.00 (br, J = 7.2 Hz, 3H). LC-MS: [M + H]⁺ = 584 | Step 1: intermediate (G1) Step 3: 4-iodo-1,3- dimethylpyridin- 2(1H)-one was replaced with intermediate (G13) |
| 175 | 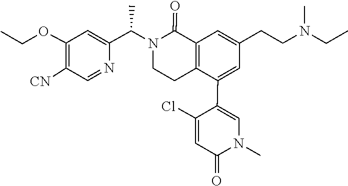 (S)-6-(1-(5-(4-chloro-1-methyl-6- oxo-1,6-dihydropyridin-3-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ ppm 8.51 (d, J = 7.3 Hz, 1H), 7.85 (d, J = 1.6 Hz, 1H), 7.61 (d, J = 14.1 Hz, 1H), 7.23 (s, 1H),7.09 (d, J = 1.2 Hz, 1H), 6.64 (d, J = 5.3 Hz, 1H), 5.87 (dq, J = 2.4, 7.1 Hz, 1H), 4.26 - 4.16 (m, 2H), 3.53- 3.33 (m, 5H), 2.90 -2.74 (m, 4H), 2.66 (q, J = 7.1 Hz, 4H), 2.40 (s , 3H), 1.56 (dd, J = 4.3, 7.1 Hz, 3H), 1.37 (t, J = 7.0 Hz, 3H), 1.08 (t, J = 7.2 Hz, 3H). LC-MS: [M + H]⁺ = 548 | Step 1: intermediate (G1) Step 3: 4-iodo-1 ,3- dimethylpyridin- 2(1H)-one was replaced with 5- bromo-4-chloro-1- methylpyridin- 2(1H)-one |
| 176 | 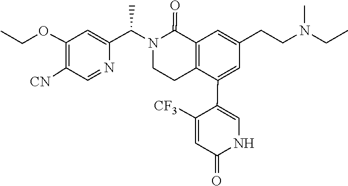 (S)-4-ethoxy-6-(1-(7-(2- (ethyl(methyl)amino)ethyl)-1-oxo-5- (6-oxo-4-(trifluoromethyl)-1,6- dihydropyridin-3-yl)-3,4- dihydroisoquinolin-2(1H)- yl)ethyl)nicotinonitrile | ¹H NMR (400 MHz, Methanol-d₄) δ ppm 8.63 (s, 1H), 7.95 (d, J = 1.6 Hz, 1H), 7.44 (d, J = 15.2 Hz, 1H), 7.33 (s, 1H), 7.35 -7.30 (m, 1H), 7.20 (d, J = 5.6 Hz, 1H), 6.94 (d, J = 3.6 Hz, 1H), 5.99 (quin, J = 7.0 Hz, 1H), 4.37 - 4.28 (m, 2H), 3.65- 3.48 (m,3H), 2.94 - 2.87 (m, 2H), 2.81 - 2.70 (m, 3H), 2.61 (q, J = 7.2 Hz, 3H), 2.38 (s, 3H), 1.67 (dd, J = 3.2, 7.2 Hz, 3H), 1.49 (dt, J = 3.2,7.0 Hz, 3H), 1.14 (t, J = 7.2 Hz, 3H). LC-MS: [M + H]⁺ = 568. | Step 1: intermediate (G1) Step 3: 4-iodo-1,3- dimethylpyridin- 2(1H)-one was replaced with 5- bromo-4- (trifluoromethyl)pyr idin-2(1H)-one |
| 177 | 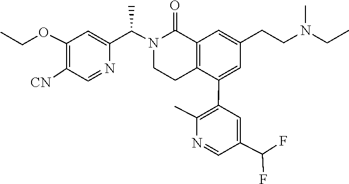 (S)-6-(1-(5-(5-(difluoromethyl)-2- methylpyridin-3-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d4) δ ppm 8.67 (s, 1H), 8.61 (d, J = 3.2 Hz, 1H), 8.02 (d, J = 1.8 Hz, 1H), 7.77 (d, J = 11.6 Hz, 1H), 7.37 (d, J = 1.8 Hz, 1H), 7.20 (d, J = 4.6 Hz, 1H), 7.10 - 6.71 (m, 1H), 5.97 (dq, J = 2.7, 7.2 Hz, 1H), 4.37 -4.24 (m, 2H), 3.65 -3.43 (m, 2H), 3.29 - 3.07 (m, 6H), 2.81 (s, 3H), 2.71 - 2.50 (m, 2H), 2.35 (d, J = 16.6 Hz, 3H), 1.66 (dd, J = 2.6, 7.2 Hz, 3H), 1.48(dt, J = 1.2, 7.0 Hz, 3H), 1.31 (t, J = 7.6 Hz, 4H). LC-MS: [M + H]⁺ = 548 | Step 1: intermediate (G1) Step 3: 4-iodo-1,3- dimethylpyridin- 2(1H)-one was replaced with 3- bromo-5- (difluoromethyl)-2- methylpyridine |
| 178 | 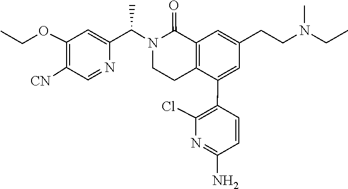 (S)-6-(1-(5-(6-amino-2- chloropyridin-3-yl)-7-(2- (ethyl(methyl)amino)ethyl)-1-oxo- 3,4-dihydroisoquinolin-2(1H)- yl)ethyl)-4-ethoxynicotinonitrile | 1H NMR (400 MHz, Methanol-d4) δ 8.62 (d, J = 8.4 Hz, 1H), 7.89 (s, 1H), 7.36 ( m, 1H), 7.26 (d, J = 1.6 Hz, 1H), 7.18 (d, J = 2.2 Hz, 1H), 6.58 (dd, J = 5.4, 8.4 Hz, 1H), 6.03 - 5.92 (m, 1H), 4.35 - 4.27 (m, 2H), 3.62 - 3.40 (m, 3H), 2.96 - 2.87 (m, 2H),2.82 - 2.62 (m, 6H), 2.42 (s, 3H), 1.66 (m, 3H), 1.47 (t, J = 7.0 Hz, 3H), 1.14 (t, J = 7.2 Hz, 3H). LC-MS: [M + H]⁺ = 533 | Step 1: intermediate (G1) Step 3: 4-iodo-1,3- dimethylpyridin- 2(1H)-one was replaced with 5- bromo-6- chloropyridin-2- amine |
| 179 | 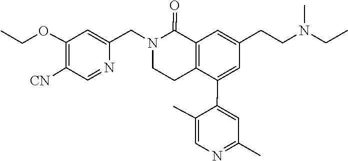 6-((5-(2,5-dimethylpyridin-4-yl)-7-(2- (ethyl(methyl)amino)ethyl)-1-oxo- 3,4-dihydroisoquinolin-2(1H)- yl)rnethyl)-4-ethoxynicotinonitrile | 1H NMR (400 MHz, Methanol-d4) δ 8.59 (s, 1H), 8.35 (s, 1H), 7.96 (d, J = 1.8 Hz, 1H), 7.26 (d, J = 1.8 Hz, 1H), 7.18 (s, 1H), 7.09(5 1H), 4.88 (d, J = 1.5 Hz, 2H), 4.31 (q, J = 7.0 Hz, 2H), 3.64 (t, J = 6.7 Hz, 2H), 2.96 (d, J = 4.0 Hz, 4H), 2.74 (d, J = 13.5 Hz, 3H), 2.65 (s, 1H), 2.51 (d, J = 5.8 Hz, 6H), 2.06 (s, 3H), 1.47 (t, J = 7.0 Hz, 3H), 1.19 (d, J = 7.6 Hz, 3H). LC-MS: [M + H]⁺ = 498 | Step 1: intermediate (G1) was replaced with intermediate (G2) Step 3: 4-iodo-1,3- dimethylpyridin- 2(1H)-one was replaced with 4- bromo-2,5- dimethylpyridine |
| 180 | 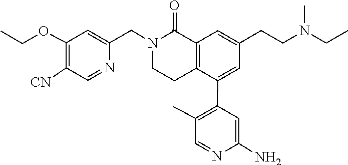 6-((5-(2-amino-5-methylpyridin-4- yl)-7-(2-(ethyl(methyl)amino)ethyl)- 1-oxo-3,4-dihydroisoquinolin-2(1H)- yl)rnethyl)-4-ethoxynicotinonitrile | ¹H NMR (400 MHz, Methanol-d₄) δ 8.60 (s, 1H), 8.05 (d, J = 1.8 Hz, 1H), 7.81 (s, 1H), 7.39 (d, J = 1.8 Hz, 1H), 7.22 (s, 1H), 6.83 (s, 1H), 4.90 (br s, 2H), 4.34 (q, J = 7.0 Hz, 2H), 3.71 -3.64 (m, 2H), 3.52 - 3.35 (m, 4H), 3.26 - 3.11 (m, 3H), 2.94 (s, 3H), 2.91- 2.80 (m, 1H), 2.77 - 2.66 (m, 1H), 1.96 (s, 3H), 1.48 (t, J = 7.0 Hz, 3H), 1.37 (t, J = 7.2 Hz, 3H). LC-MS: [M + H]⁺ = 499 | Step 1: intermediate (G1) was replaced with intermediate (G2) Step 3: 4-iodo-1,3- dimethylpyridin- 2(1H)-one was replaced with 4- bromo-5- methylpyridin-2- amine |
| 181 | 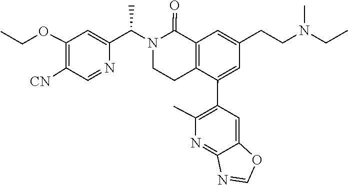 (S)-4-ethoxy-6-(1-(7-(2- (ethyl(methyl)amino)ethyl)-5-(5- methyloxazolo[4,5-b]pyridin-6-yl)-1- oxo-3,4-dihydroisoquinolin-2(1H)- yl)ethyl)nicotinonitrile | ¹H NMR (400 MHz, Methanol-d₄) δ 8.75 (d,J = 2.2 Hz, 1H), 8.62 (d,J = 3.8 Hz, 1H), 8.00 (s, 1H), 8.05 - 7.92 (m, 1H), 7.96(d,J = 11.6 Hz, 1H), 7.35 (d,J = 1.7 Hz, 1H), 7.21 (d,J = 3.8 Hz, 1H), 6.05- 5.97 (m, 1H), 4.33 (dq,J = 3.4, 7.0 Hz, 2H), 3.62 -3.53 (m, 1H), 3.51 - 3.43 (m, 1H), 2.97 - 2.89 (m, 2H), 2.78 - 2.51 (m, 7H), 2.41 (s, 1H), 2.39 - 2.32 (m, 5H), 1.67 (dd,J = 2.3,7.2 Hz, 3H), 1.49 (t,J = 7.0 Hz,3H), 1.12 (t,J = 7.2 Hz, 3H). LC-MS: [M + H]⁺ = 539 | Step 1: intermediate (G1) Step 3: 4-iodo-1,3- dimethylpyridin- 2(1H)-one was replaced with 6- bromo-5- methyloxazolo[4,5 -b]pyridine |
| 182 | 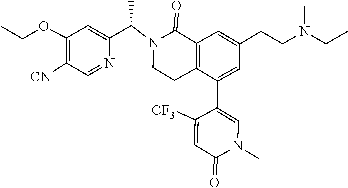 (S)-4-ethoxy-6-(1-(7-(2- (ethyl(methyl)amino)ethyl)-5-(1- methyl-6-oxo-4-(trifluoromethyl)-1,6- dihydropyridin-3-yl)-1-oxo-3,4- dihydroisoquinolin-2(1H)- yl)ethyl)nicotinonitrile | ¹H NMR (400 MHz, Methanol-d₄) δ 8.61 (s, 1H), 7.93 (d, J = 1.9 Hz, 1H), 7.69 (d, J = 17.0 Hz, 1H), 7.37 - 7.26 (m, 1H), 7.18 (d, J = 6.2 Hz, 1H), 6.93 (d, J = 3.5 Hz, 1H), 5.97 (p, J = 7.1 Hz, 1H), 4.30 (qdd, J = 6.9, 3.4, 1.4 Hz, 2H), 3.61 (d, J = 8.2 Hz, 3H), 3.58- 3.34 (m, 2H), 2.91 - 2.71 (m, 3H), 2.69 - 2.50 (m, 5H), 2.32 (s, 3H), 1.65 (dd, J = 7.1, 3.5 Hz, 3H), 1.47 (td, J = 7.0, 4.4 Hz, 3H), 1.10 (t, J = 7.2 Hz, 3H). LC-MS: [M + H]⁺ = 582 | Step 1: intermediate (G1) Step 3: 4-iodo-1,3- dimethylpyridin- 2(1H)-one was replaced with 5- bromo-1-methyl-4- (trifluoromethyl)pyr idin-2(1H)-one |
| 183 | 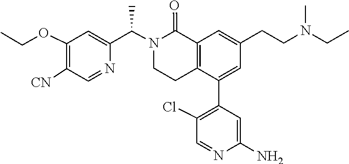 (S)-6-(1-(5-(2-amino-5- chloropyridin-4-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ 8.61 (d, J = 7.3 Hz, 1H), 7.99 - 7.91 (m, 2H), 7.29 - 7.16 (m, 2H), 6.47 (d, J = 12.6 Hz, 1H), 6.03- 5.94 (m, 1H), 4.31 (q, J = 7.1 Hz, 2H), 3.59 - 3.45 (m, 2H), 2.89 (dd, J = 10.2, 6.0 Hz, 2H), 2.78 - 2.65 (m, 4H), 2.56 (q, J = 7.2 Hz, 2H), 2.34 (s, 3H), 1.65 (dd, J = 7.1, 3.7 Hz, 3H), 1.47 (t, J = 7.0 Hz, 3H), 1.11 (t, J = 7.2 Hz, 3H). LC-MS: [M + H]⁺ = 533 | Step 1: intermediate (G1) Step 3: 4-iodo-1,3- dimethylpyridin- 2(1H)-one was replaced with 5- bromo-6- chloropyridin-3- amine |
| 184 | 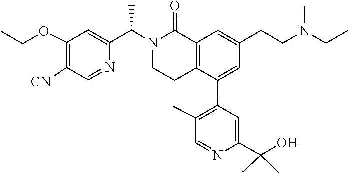 (S)-4-ethoxy-6-(1-(7-(2- (ethyl(methyl)amino)ethyl)-5-(2-(2- hydroxypropan-2-yl)-5- methylpyridin-4-yl)-1-oxo-3,4- dihydroisoquinolin-2(1H)- yl)ethyl)nicotinonitrile | ¹H NMR (400 MHz, Methanol-d₄) δ 8.63 (s, 1H), 8.44 (d, J = 4.4 Hz, 1H), 8.00 (s, 1H), 7.44 (d, J = 13.4 Hz, 1H), 7.31 (d,J = 1.8 Hz, 1H), 7.21 (d, J = 2.8 Hz, 1H), 6.05 - 5.94 (m, 1H), 4.39 - 4.29 (m, 2H), 3.64 - 3.43 (m, 2H), 3.16 - 3.01 (m, 5H), 2.96(q, J = 7.2 Hz, 2H), 2.67 (s, 3H), 2.10 (d, J = 17.0 Hz, 3H),1.67(d, J = 7.2Hz, 3H), 1.57 (t, J = 4.6 Hz, 7H), 1.50(t, J = 7.0 Hz, 3H),1.26 (t, J = 7.2 Hz, 3H) LC-MS:[M + H]⁺ = 556 | Step 1: intermediate (G1 Step 3: 4-iodo-1,3- dimethylpyridin- 2(1H)-one was replaced with intermediate (G14) |
| 185 | 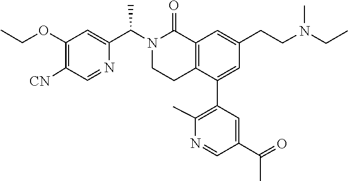 (S)-6-(1-(5-(5-acetyl-2- methylpyridin-3-yl)-7-(2- (ethyl(methyl)amino)ethyl)-1-oxo- 3,4-dihydroisoquinolin-2(1H)- yl)ethyl)-4-ethoxynicotinonitrile | 1H NMR (400 MHz, Methanol-d4) δ 9.04 (d, J = 2.1 Hz, 1H), 8.61 (d, J = 2.9 Hz, 1H), 8.09 (dd, J = 2.2,12.7 Hz, 1H), 7.98 (d, J = 1.5 Hz, 1H), 7.30 (d, J = 1.7 Hz, 1H), 7.19 (d, J = 4.5 Hz, 1H), 6.04- 5.93 (m, 1H), 4.32 (dq, J = 2.9, 7.0 Hz, 2H), 3.62 - 3.51 (m, 1H), 3.49 -3.41 (m, 1H), 2.95 - 2.87 (m, 2H), 2.76 - 2.68 (m, 2H), 2.65 - 2.52 (m, 7H), 2.40 - 2.33 (m, 6H), 1.65 (dd, J = 2.4, 7.2 Hz, 3H), 1.47 (t, J = 7.0 Hz, 3H), 1.11 (t, J = 7.2 Hz, 3H) LC-MS: [M + H]⁺ = 540 | Step 1: intermediate (G1) Step 3:4-iodo-1,3- dimethylpyridin- 2(1H)-one was replaced with 1-(5- chloro-6- methylpyridin-3- yl)ethan-1-one |
| 186 | 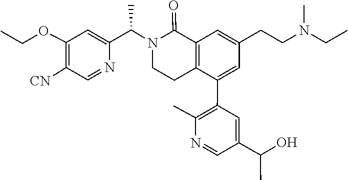 4-ethoxy-6-((1S)-1-(7-(2- (ethyl(methyl)amino)ethyl)-5-(5-(1- hydroxyethyl)-2-methylpyridin-3-yl)- 1-oxo-3,4-dihydroisoquinolin-2(1H)- yl)ethyl)nicotinonitrile | ¹H NMR (400 MHz, Methanol-d₄) δ 8.61 (d, J = 2.4 Hz, 1H), 8.49- 8.43 (m, 1H), 7.95 (d, J = 1.6 Hz, 1H),7.62 - 7.54 (m, 1H), 7.30 - 7.24 (m, 1H), 7.19 (d, J = 2.9 Hz, 1H), 6.03 - 5.93 (m, 1H), 4.95 - 4.89 (m, 1H), 4.31 (dq, J = 2.5, 7.0 Hz, 2H), 3.61 -3.50 (m, 1H), 3.49 -3.40 (m, 1H), 2.95 - 2.86 (m, 2H), 2.76 - 2.67 (m, 2H), 2.63 - 2.52 (m, 4H), 2.34 (s, 3H), 2.27 (d, J = 17.7 Hz, 3H), 1.65 (d, J = 7.0 Hz, 3H), 1.48 (dt, J = 3.9, 6.6 Hz, 6H), 1.11 (t, J = 7.2 Hz, 3H). LC-MS: [M + H]⁺ = 542 | Obtained by reduction of compound 185 |
| 187 | 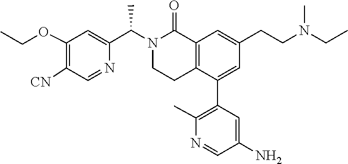 (S)-6-(1-(5-(5-amino-2- methylpyridin-3-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ 8.61 (d, J = 3.2 Hz, 1H), 7.94 - 7.88 (m, 2H), 7.21 (dd, J = 20.3, 1.9 Hz, 2H), 6.86 (dd, J = 13.2, 2.7 Hz, 1H), 5.98 (qd, J = 7.1, 3.1 Hz, 1H), 4.39 - 4.25 (m, 2H), 3.61 - 3.38 (m, 2H), 2.94 - 2.84 (m, 2H), 2.76 - 2.51 (m, 6H), 2.33 (s , 3H), 2.10 (d, J = 17.9 Hz, 3H), 1.64 (dd, J = 7.1, 2.1 Hz, 3H), 1.47 (td, J = 7.0, 0.7 Hz, 3H), 1.11 (t, J = 7.2 Hz, 3H). LC-MS: [M + H]⁺ = 513. | Step 1: intermediate (G1) Step 3: 4-iodo-1,3- dimethylpyridin- 2(1H)-one was replaced with 5- bromo-6- methylpyridin-3- amine |
| 188 | 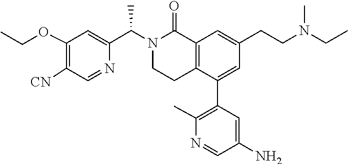 (S)-4-ethoxy-6-(1-(7-(2- (ethyl(methyl)amino)ethyl)-5-(2- methoxy-5-methylpyridin-4-yl)-1- oxo-3,4-dihydroisoquinolin-2(1H)- yl)ethyl)nicotinonitrile | ¹H NMR (400 MHz, Methanol-d₄) δ 8.63 (s, 1H), 8.06 (d, J = 4.6 Hz, 1H), 7.95 (s, 1H), 7.24 (d, J = 1.6 Hz, 1H), 7.20 (d,J = 4.8 Hz, 1H), 6.61 (d, J = 11.1 Hz, 1H), 6.00 (m, J = 2.8, 7.1 Hz, 1H), 4.33 (m, J = 3.1, 7.0 Hz, 2H), 3.92 (d, J = 3.5 Hz, 3H), 3.6-3.52 (m, 1H), 3.52- 3.41 (m, 1H), 2.96-2.88 (m, 2H), 2.79-2.72 (m, 2H), 2.69-2.55 (m, 3H), 2.71 (brs, 1H), 2.71-2.50 (m,1H), 2.39 (s, 3H), 1.98 (d, J = 17.1 Hz, 3H), 1.66 (d, J = 7.1 Hz, 3H), 1.49 (t, J = 7.0 Hz, 3H), 1.14 (t, J = 7.2 Hz, 3H). LC-MS: [M + H]⁺ = 528. | Step 1: intermediate (G1) Step 3: 4-iodo-1,3- dimethylpyridin- 2(1H)-one was replaced with 4- bromo-2-methoxy- 5-methylpyridine |
| 189 | 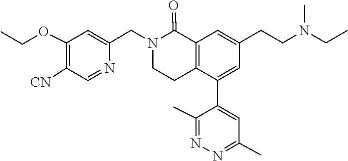 6-((5-(3,6-dimethylpyridazin-4-yl)-7- (2-(ethyl(methyl)amino)ethyl)-1-oxo- 3,4-dihydroisoquinolin-2(1H)- yl)methyl)-4-ethoxynicotinonitrile | ¹H NMR (400 MHz, Methanol-d₄) δ 8.59 (s, 1H), 8.39 (s, 1H), 8.05 (d, J = 1.9 Hz, 1H), 7.44 (s, 1H), 7.39 (d, J = 2.0 Hz, 1H), 7.20 (5 s, 1H), 4.90(d, J = 2.5 Hz, 2H), 4.31 (q, J = 7.0 Hz, 2H), 3.72 - 3.61 (m, 2H), 3.43 - 3.34 (m, 2H), 3.24 (q, J = 7.3 Hz, 2H), 3.15 (dd, J = 10.6, 6.2 Hz, 2H), 2.89 (s, 3H), 2.78 (ddd, J = 16.2, 7.9, 5.8 Hz, 1H), 2.69 (s, 4H), 2.44 (s, 3H), 1.48 (t, J = 7.0 Hz, 3H), 1.35 (t, J = 7.3 Hz, 3H). LC-MS:[M + H]⁺ = 499. | Step 1: intermediate (G1) Step 3: 4-iodo-1,3- dimethylpyridin- 2(1H)-one was replaced with 4- chloro-3,6- dimethylpyridazine |
| 190 | 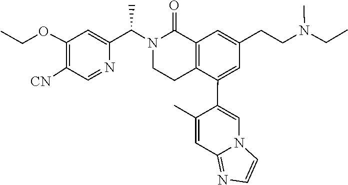 (S)-4-ethoxy-6-(1-(7-(2- (ethyl(methyl)amino)ethyl)-5-(7- methylimidazo[1,2-a]pyridin-6-yl)-1- oxo-3,4-dihydroisoquinolin-2(1H)- yl)ethyl)nicotinonitrile | ¹H NMR (400 MHz, Methanol-d₄) δ 8.60 (d, J = 4.3 Hz, 1H), 8.27 (d, J = 12.7 Hz, 1H), 8.02 (s, 1H), 7.78 (d, J = 7.0 Hz, 1H), 7.59 - 7.51 (m, 1H), 7.49 (d, J = 4.7 Hz, 1H), 7.43 (d, J = 1.7 Hz, 1H), 7.20 (d, J = 4.0 Hz, 1H), 5.97 (dd, J = 7.1, 3.9 Hz, 1H), 4.31 (ttd, J = 6.9, 4.4, 2.1 Hz, 2H), 3.63 -3.43 (m, 2H), 3.38 (dd, J = 10.2, 6.4 Hz, 2H), 3.25 (q, J = 7.3 Hz, 2H), 3.20 - 3.08 (m, 2H), 2.90 (s, 3H), 2.79 - 2.72 (m, 1H), 2.63 (ddd, J = 16.3, 7.7, 2.7 Hz, 1H), 2.11 (d, J = 16.8 Hz, 3H), 1.65 (dd, J = 7.1, 2.1 Hz, 3H), 1.47 (td, J = 7.0, 1.3 Hz, 3H), 1.35 (t, J = 7.3 Hz, 3H). LC-MS: [M + H]⁺ = 537. | Step 1: intermediate (G1) Step 3: 4-iodo-1,3- dimethylpyridin- 2(1H)-one was replaced with 6- bromo-7- methylimidazo[1,2 -a]pyridine |
| 191 | 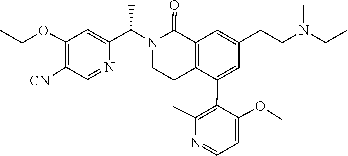 4-ethoxy-6-((1S)-1-(7-(2- (ethyl(methyl)amino)ethyl)-5-(4- methoxy-2-methylpyridin-3-yl)-1- oxo-3,4-dihydroisoquinolin-2(1H)- yl)ethyl)nicotinonitrile | ¹H NMR (400 MHz, Methanol-d₄) δ 8.61 (d, J = 5.5 Hz, 1H), 8.36 (br d, J = 5.9 Hz, 1H), 8.03- 7.88 (m, 1H), 7.33 - 7.16 (m, 2H), 7.08- 6.98 (m, 1H), 6.05 - 5.92 (m, 1H), 4.37 - 4.27 (m, 2H), 3.79 (d, J = 13.6 Hz, 3H), 3.56- 3.50 (m, 1H), 3.48- 3.39 (m,2H), 3.12(5 , 1H), 2.98 - 2.88 (m, 1H), 2.87 - 2.76 (m, 1H), 2.69 (q, J = 7.0 Hz, 1H), 2.59 - 2.46 (m, 2H), 2.44 (s, 1H), 2.46- 2.41(m, 1H), 2.24 - 2.12 (m, 3H), 1.65 (dd, J = 3.5, 7.1 Hz, 3H), 1.47 (dt, J = 3.5, 6.9 Hz, 3H), 1.15 (br t, J = 7.2 Hz, 3H). LC-MS: [M + H]⁺ = 528 | Step 1: intermediate (G1) Step 3: 4-iodo-1,3- dimethylpyridin- 2(1H)-one was replaced with 3- bromo-4-methoxy- 2-methylpyridine |
| 192 | 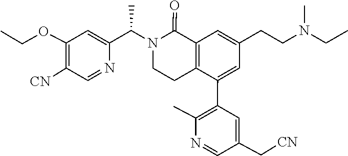 (S)-6-(1-(5-(5-(cyanomethyl)-2- methylpyridin-3-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ 8.61 (d, J = 3.1 Hz, 1H), 8.49 (d, J = 1.8 Hz, 1H), 7.98 (d, J = 1.3 Hz, 1H), 7.61 (dd, J = 2.1, 12.7 Hz, 1H), 7.31 (s, 1H), 7.19 (d, J = 3.9 Hz, 1H), 6.02 - 5.93 (m, 1H),4.56 (br s, 1H), 4.39 - 4.25 (m, 2H), 3.99 (d, J = 5.7 Hz, 1H), 3.63 - 3.52 (m, 1H), 3.51 - 3.42 (m, 1H), 3.02 - 2.87 (m, 4H), 2.78 (q, J = 7.2 Hz, 2H), 2.68 - 2.54 (m, 2H), 2.51 (s, 3H), 2.30 (d, J = 18.1 Hz, 3H),1.94 (br s, 1H), 1.65 (dd, J = 1.8, 7.1 Hz, 3H), 1.47 (t, J = 6.7 Hz, 3H), 1.18 (t, J = 7.2 Hz, 3H). LC-MS: [M + H]⁺ = 537. | Step 1: intermediate (G1) Step 3: 4-iodo-1,3- dimethylpyridin- 2(1H)-one was replaced with 2-(5- bromo-6- methylpyridin-3- yl)acetonitrile |
| 193 | 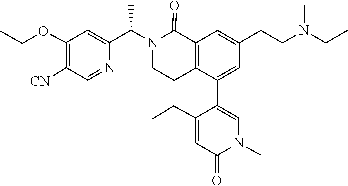 (S)-4-ethoxy-6-(1-(7-(2- (ethyl(methyl)amino)ethyl)-5-(4- ethyl-1-methyl-6-oxo-1,6- dihydropyridin-3-yl)-1-oxo-3,4- dihydroisoquinolin-2(1H)- yl)ethyl)nicotinonitrile | ¹H NMR (400 MHz, Methanol-d₄) δ 8.65 (s, 1H), 8.67-8.60 (m, 1H), 8.63 (s, 1H), 8.64-8.60 (m, 1H), 7.95 (s, 1H), 7.99-7.90 (m, 1H), 7.46 (d, J = 14.7 Hz, 1H), 7.38 (s, 1H), 7.32 (d, J = 1.3 Hz, 1H), 7.21 (d, J = 2.4 Hz, 1H), 7.19 (s, 1H), 7.23-7.17(m, 1H), 6.87 (s, 1H), 6.53 (d, J = 4.4 Hz, 1H), 6.57-6.49 (CR 1H), 6.58-6.46 (m, 1H), 6.04- 5.96 (m, 1H), 4.33 (q, J = 6.5 Hz,2H), 3.64-3.39 (m, 5H),2.99- 2.88 (m, 2H), 2.86-2.57 (m, 7H), 2.40 (brs, 1H), 2.44 (s, 2H), 2.36- 2.17 (m, 2H), 1.67 (d,J = 7.2 Hz, 3H), 1.52-1.46 (m, 3H),1.16 (t, J = 7.2 Hz, 3H), 1.03 (td, J = 7.5, 11.4 Hz, 3H). LC-MS: [M + H]⁺ = 542. | Step 1: intermediate (G1) Step 3: 4-iodo-1,3- dimethylpyridin- 2(1H)-one was replaced with 5- bromo-4-ethyl-1- methylpyridin- 2(1H)-one |
| 194 | 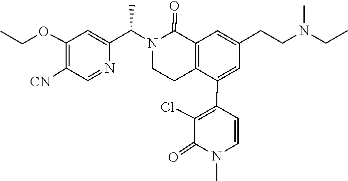 (S)-6-(1-(5-(3-chloro-1-methyl-2- oxo-1,2-dihydropyridin-4-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ 8.61 (s, 1H), 8.53 (s, 1H), 8.35 (d, J = 4.1 Hz, 1H), 7.96 (d, J = 1.9 Hz, 1H), 7.22 (dd, J = 27.6, 2.6 Hz, 2H), 7.07 (d, J = 12.3 Hz, 1H), 6.05 - 5.91 (m, 1H), 4.47 (q, J = 5.3 Hz, 1H), 4.31 (dt, J = 7.9, 6.2 Hz, 2H), 4.11 (d, J = 18.9 Hz, 1H), 3.81 (s, 1H), 3.69 - 3.41 (m, 3H), 3.28 - 3.01 (m, 2H), 2.90 (q, J = 9.8, 9.3 Hz, 2H), 2.72 -2.46 (m, 5H), 2.04 (d, J = 16.7 Hz, 3H), 1.65 (d, J = 7.1 Hz, 3H), 1.47 (t, J = 7.0 Hz, 3H), 1.38 (d, J = 6.7 Hz, 3H). LC-MS: [M + H]⁺ = 548. | Step 1: intermediate (G1) Step 3: 4-iodo-1,3- dimethylpyridin- 2(1H)-one was replaced with 4- bromo-1,3- dimethylpyridin- 2(1H)-one |
| 195 | 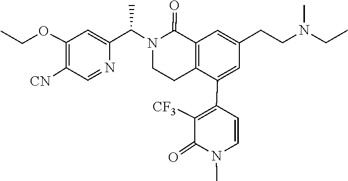 (S)-4-ethoxy-6-(1-(7-(2- (ethyl(methyl)amino)ethyl)-5-(1- methyl-2-oxo-3-(trifluoromethyl)-1,2- dihydropyridin-4-yl)-1-oxo-3,4- dihydroisoquinolin-2(1H)- yl)ethyl)nicotinonitrile | ¹H NMR (400 MHz, Methanol-d₄) δ 8.64 (d, J = 7.6 Hz, 1H), 7.97- 7.88 (m, 2H), 7.23 (dd, J = 1.8, 3.9 Hz, 1H), 7.20 (d,J = 5.6 Hz, 1H), 6.18 (dd, J = 6.8, 14.7 Hz, 1H), 5.99 (dq, J = 3.5, 7.0 Hz, 1H), 4.38 4.28 (m, 2H), 3.65(s, 3H), 3.6 -3 3.46 (m,2H), 2.97 - 2.85 (m, 2H), 2.78 -2.64 (m, 4H), 2.59 (q, J = 7.2 Hz, 2H),2.36 (s, 3H), 1.67 (dd, J = 2.3, 7.1 Hz, 3H), 1.49 (dt, J = 2.9,7.0 Hz, 3H), 1.13 (t, J = 7.2 Hz, 3H). LC-MS:[M + H]⁺ = 582. | Step 1: intermediate (G1) Step 3: 4-iodo-1,3- dimethylpyridin- 2(1H)-one was replaced with intermediate (G15) |
| 197 | 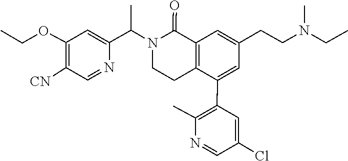 6-(1-(5-(5-chloro-2-methylpyridin-3- yl)-7-(2-(ethyl(methyl)amino)ethyl)- 1-oxo-3,4-dihydroisoquinolin-2(1H)- yl)ethyl)-4-ethoxynicotinonitrile | ¹H NMR (400 MHz, Methanol-d₄) δ 8.61 (d, J = 1.6 Hz, 1H), 8.53(5 0H), 8.48 (d, J = 2.4 Hz, 1H), 7.98 Step 3: 4-iodo-1,3- (d, J = 1.9 Hz, 1H), 7.67 (dd, J = 10.5, 2.5 Hz, 1H), 7.32 (d, J = 1.9 Hz, 1H), 7.19 (d, J = 4.8 Hz, 1H), 5.97 (dd, J = 7.1, 2.5 Hz, 1H), 4.30 (td, J = 7.0, 2.6 Hz, 2H), 3.60 - 3.44 (m, 2H), 3.00 (s, 4H), 2.87 (d, J = 7.8 Hz, 2H), 2.59 (s, 5H), 2.26 (d, J = 16.9 Hz, 3H), 1.65 (dd, J = 7.2, 2.0 Hz, 3H), 1.47 (t, J = 7.0 Hz, 3H), 1.21 (t, J = 7.2 Hz, 3H). LC-MS: [M + H]⁺ = 532. | Step 1: intermediate (G1) dimethylpyridin- 2(1H)-one was replaced with 3- bromo-5-chloro-2- methylpyridine |
| 198 | 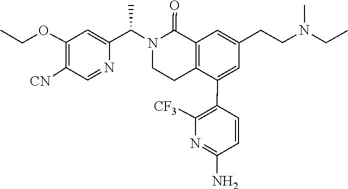 (S)-6-(1-(5-(6-amino-2- (trifluoromethyl)pyridin-3-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ 8.61 (d, J = 2.1 Hz, 1H), 7.89 (s, 1H), 7.33 (dd, J = 8.6, 14.4 Hz, 1H), 7.21 (s, 1H), 7.17 (d, J = 6.4 Hz, 1H), 6.78 (t, J = 7.9 Hz, 1H), 6.03 - 5.93 (m, 1H), 4.38 - 4.23 (m, 2H), 3.61 - 3.36 (m, 2H), 2.92 - 2.82 (m, 2H), 2.74 - 2.61 (m, 3H), 2.57 (q, J = 7.3 Hz, 3H), 2.34 (s, 3H), 1.64 (dd, J = 1.8, 7.2 Hz, 3H), 1.47 (dt, J = 3.5, 7.0 Hz, 3H), 1.11 (t, J = 7.2 Hz, 3H). LC-MS: [M + H]+ = 567. | Step 1: intermediate (G1) Step 3: 4-iodo-1,3- dimethylpyridin- 2(1H)-one was replaced with 5- bromo-6- (trifluoromethyl)pyr idin-2-amine |
| 199 | 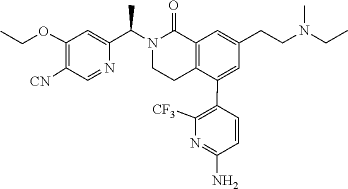 (R)-6-(1-(5-(6-amino-2- (trifluoromethyl)pyridin-3-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ 8.61 (d, J = 2.2 Hz, 1H), 7.89 (s, 1H), 7.33 (dd, J = 8.6, 14.6 Hz, 1H), 7.21 (s, 1H), 7.17 (d, J = 6.2 Hz, 1H), 6.78 (t, J = 8.0 Hz, 1H), 6.01 - 5.93 (m, 1H), 4.36 - 4.24 NN (m, 2H), 3.61 - 3.37 (m, 2H), 2.91 - 2.82 (m, 2H), 2.71 - 2.60 (m, 3H), 2.56 (q, J = 7.2 Hz, 3H), 2.33 (s, 3H), 1.68 -1.60 (m, 3H), 1.47 (dt, J = 3.5, 7.0 Hz, 3H), 1.10 (t, J = 7.2 Hz, 3H) LC-MS:[M + H]⁺ = 567. | Step 1: intermediate (G1) Step 3: 4-iodo-1 3- dimethylpyridin- 2(1H)-one was replaced with 5- bromo-6- (trifluoromethyl)pyr idin-2-amine |
| 200 | 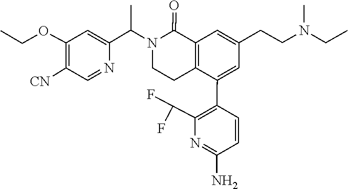 6-(1-(5-(6-amino-2- (difluoromethyl)pyridin-3-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ 8.61 (d, J = 1.4 Hz, 1H), 7.98 (d, J = 1.9 Hz, 1H), 7.58 (d, J = 16.6 Hz, 1H), 7.45 - 7.31 (m, 2H), 7.19 (d, J = 4.6 Hz, 1H), 6.81 (ddt, J = 8.4, 7.3, 1.1 Hz, 1H), 6.34 (td, J = 53.9, 16.4 Hz, 1H), 5.96 (qd, J = 7.1, 2.8 Hz, 1H), 4.31 (qdd, J = 7.0, 3.4, 1.7 Hz, 2H), 3.63 - 3.34 (m, 4H), 3.26 - 3.03 (m, 4H), 2.93 (s, 3H), 2.79 - 2.56 (m, 2H), 1.65 (dd, J = 7.2, 3.6 Hz, 3H), 1.47 (td, J = 7.0, 2.2 Hz, 3H), 1.35 (t, J = 7.3 Hz, 3H). LC-MS: [M + H]⁺ = 549. | Step 1: intermediate (G1) Step 3: 4-iodo-1,3- dimethylpyridin- 2(1H)-one was replaced with 5- bromo-6- (difluoromethyl)pyr idin-2-amine |
| 201 | 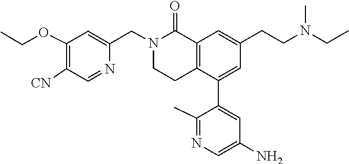 6-((5-(5-amino-2-methylpyridin-3- yl)-7-(2-(ethyl(methyl)amino)ethyl)- 1-oxo-3,4-dihydroisoquinolin-2(1H)- yl)methyl)-4-ethoxynicotinonitrile | ¹H NMR (400 MHz, Methanol-d₄) δ 8.59 (s, 1H), 8.40 (s, 2H), 7.98 (d, J = 1.9 Hz, 1H), 7.93(d, J = 2.7 Hz, 1H), 7.36 (d, J = 2.0 Hz, 1H), 7.19 (s, 1H), 6.91 (d, J = 2.7 Hz, 1H), 4.89 (d, J = 1.5 Hz, 2H), 4.31 (q, J = 7.0 Hz, 2H), 3.66 (dd, J = 7.4, 5.9 Hz, 2H), 3.43 - 3.36 (m, 2H), 3.26 (t, J = 7.3 Hz, 2H), 3.19- 3.10 (m, 2H), 2.91 (s, 3H), 2.74 (qt, J = 16.3, 6.2 Hz, 2H), 2.12 (s, 3H), 1.47 (t, J = 7.0 Hz, 3H), 1.35 (t, J = 7.3 Hz, 3H). LC-MS: [M + H]⁺ = 499. | Step 1: intermediate (G1) was replaced with intermediate (G2) Step 3: 4-iodo-1,3- dimethylpyridin- 2(1H)-one was replaced with 5- bromo-6- methylpyridin-3- amine |
| 202 | 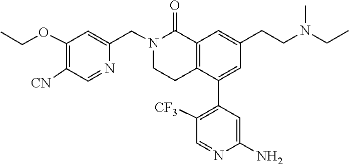 (S)-6-(1-(5-(2-amino-5- (trifluoromethyl)pyridin-4-yl)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ 8.62 (s, 1H), 8.26 (d, J = 2.9 Hz, 1H), 7.93 (d, J = 1.5 Hz, 1H), 7.25 (s, 1H), 7.18 (d, J = 8.7 Hz, 1H), 6.36 (d, J = 14.8 Hz, 1H), 5.97 (quin, J = 6.7 Hz, 1H), 4.31 (q, J = 6.7 Hz, 2H), 3.63 -3.53 (m, 1H), 3.50 (br t, J = 4.8 Hz, 1H), 3.38 (ddd, J = 4.8, 8.3, 12.9 Hz, 1H), 2.95-2.84 (m, 2H), 2.78 - 2.67 (m, 3H), 2.65 - 2.47 (m, 3H), 2.38 (s, 3H), 1.65 (dd, J = 4.3, 7.1 Hz, 3H), 1.47 (dt, J = 4.6, 7.0 Hz, 3H), 1.12 (t, J = 7.2 Hz, 3H). LC-MS: [M + H]⁺ = 567. | Step 1: intermediate (G1) Step 3: 4-iodo-1,3- dimethylpyridin- 2(1H)-one was replaced with 4- bromo-5- (trifluoromethyl)pyr idin-2-amine |
| 203 | 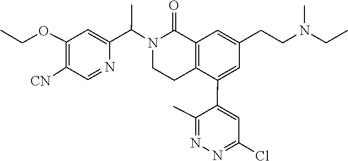 6-(1-(5-(6-chloro-3-methylpyridazin- 4-yI)-7-(2- (ethyl(methyl)amino)ethyl)-1-oxo- 3,4-dihydroisoquinolin-2(1H)- yl)ethyl)-4-ethoxynicotinonitrile | ¹H NMR (400 MHz, Methanol-d₄) δ 8.61 (s, 1H), 8.01 (d, J = 2.0 Hz, 1H), 7.68 (d, J = 10.3 Hz, 1H), 7.34 (d, J = 1.5 Hz, 1H), 7.19 (d, J = 4.6 Hz, 1H), 5.98 (qd, J = 7.1, Hz, 2H), 3.65 - 3.38 (m, 2H), 2.94 3.0 Hz, 1H), 4.31 (qd, J = 7.0, 3.1 (dd, J = 9.9, 6.1 Hz, 2H), 2.80 (dd, J = 10.2, 6.0 Hz, 2H), 2.75- 2.50 (m, 4H), 2.48 - 2.35 (m, 6H), 1.73 - 1.58 (m, 3H), 1.47 (t, J = 7.0 Hz, 3H), 1.14 (t,J = 7.2 Hz, 3H). LC-MS:[M + H]⁺ = 533. | Step 1: intermediate (G1) Step 3: 4-iodo-1,3- dimethylpyridin- 2(1H)-one was replaced with 4,6- dichloro-3- methylpyridazine |
| 204 | 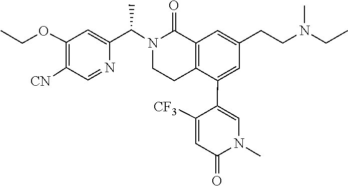 (S)-2-(1-(4-ethoxy-5-fluompyridin-2- yl)ethyl)-7-(2- (ethyl(methyl)amino)ethyl)-5-(1- methyl-6-oxo-4-(trifluoromethyl)-1,6- dihydropyridin-3-yl)-3,4- dihydroisoquinolin-1(2H)-one | ¹H NMR (400 MHz, Methanol-d₄) δ 8.22 (dd, J = 0.7, 3.2 Hz, 1H), 7.95 (d, J = 1.6 Hz, 1H), 7.67 (d, J = 19.8 Hz, 1H), 7.34-7.24 (m, 1H), 7.17-7.08 (m, 1H), 6.92 (d, J = 4.4 Hz, 1H), 6.05-5.90 (m, 1H), 4.26-4.12 (m, 2H), 3.60 (d,J = 10.8 Hz, 3H),3.56-3.37 (m, 2H), 3.30 (br s, 1H), 2.90-2.83 (m, 2H), 2.81-2.62 (m, 3H), 2.54 (q, J = 7.2 Hz, 3H), 2.31 (s, 3H)-1.61 (dd, J = 3.6, 7.2 Hz, 3H), 1.43 (dt, J = 5.2, 7.0 Hz, 3H), 1.10 (t, J = 7.2 Hz, 3H). LC-MS: [M + H]⁺ = 575. | Step 1: intermediate (G1) Step 3: 4-iodo-1,3- dimethylpyridin- 2(1H)-one was replaced with 5- bromo-1-methyl-4- (trifluoromethyl)pyr idin-2(1H)-one |
| 205 | 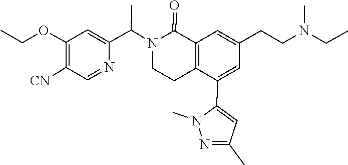 6-(1-(5-(1,3-dimethyl-1H-pyrazol-5- yl)-7-(2-(ethyl(methyl)amino)ethyl)- 1-oxo-3,4-dihydroisoquinolin-2(1H)- yl)ethyl)-4-ethoxynicotinonitrile | ¹H NMR (400 MHz, Methanol-d₄) δ 8.61 (s, 1H), 8.47 (s, 1H), 8.02 (d, J = 1.9 Hz, 1H), 7.45 (d, J = 2.0 Hz, 1H), 7.20 (s, 1H), 6.10 (d, J = 0.6 Hz, 1H), 5.96 (q, J = 7.1 Hz, 1H), 4.32 (qd, J = 7.1, 1.0 Hz, 2H), 3.64 - 3.45 (m, 5H), 3.33 (s, 2H), 3.25- 3.05 (m, 4H), 2.91 - 2.82 (m, 3H), 2.76 (t, J = 6.5 Hz, 2H), 2.26 (s, 3H), 1.66 (d, J = 7.1 Hz, 3H), 1.48 (t, J = 7.0 Hz, 3H), 1.38 - 1.27 (m, 3H). LC-MS: [M + H]⁺ = 501. | Step 1: intermediate (G1) Step 3: 4-iodo-1,3- dimethylpyridin- 2(1H)-one was replaced with (1,3- dimethyl-1H- pyrazol-5- yl)boronic acid |
| 206 | 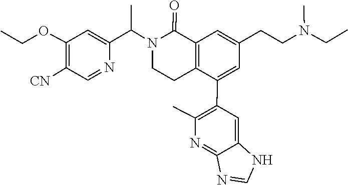 4-ethoxy-6-(1-(7-(2- (ethyl(methyl)amino)ethyl)-5-(5- methyl-1H-imidazo[4,5-b]pyridin-6- yl)-1-oxo-3,4-dihydroisoquinolin- 2(1H)-yl)ethyl)nicotinonitrile | ¹H NMR (400 MHz, Methanol-d₄) δ 8.95 (s, 1H), 8.60 (d, J = 4.8 Hz, 1H), 8.03 (d, J = 12.9 Hz, 2H), 7.43 (d, J = 1.9 Hz, 1H), 7.20 (d, J = 3.0 Hz, 1H), 5.97 (dd, J = 7.1, 3.2 Hz, 1H), 4.38 - 4.25 (m, 2H), 3.61 - 3.44 (m, 3H), 3.44 - 3.35 (m, 2H), 3.26 - 3.13 (m, 3H), 2.94 (5, 3H), 2.73 - 2.53 (m, 2H), 2.43 (d, J = 16.8 Hz, 3H), 1.65 (dd, J = 7.1, 2.3 Hz, 3H), 1.48 (td, J = 7.0, 0.8 Hz, 3H), 1.36 (t, J = 7.3 Hz, 3H). LC-MS: [M + H]⁺ = 538. | Step 1: intermediate (G1) Step 3: 4-iodo-1,3- dimethylpyridin- 2(1H)-one was replacedwith 6- bromo-5-methyl- 1H-imidazo[4,5- b]pyridine |

### Example 207: Synthesis of 4-ethoxy-6-(1-(7-(2-(ethyl(methyl)amino)ethyl)-1-oxo-5-(pyrrolidin-1-yl)-3,4-dihydroisoquinolin-2(1H)-yl)ethyl)nicotinonitrile (207)

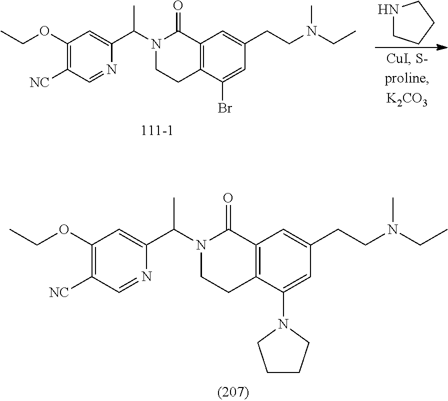

A mixture of 111-1 (50 mg, 0.103 mmol), pyrrolidine (73.3 mg, 1.030 mmol), S-proline (2.372 mg, 0.021 mmol), copper(I) iodide (5.89 mg, 0.031 mmol) and K₂CO₃ (28.5 mg, 0.206 mmol) in DMSO (2 mL) was stirred for 20 hrs. at 110° C. under N₂ atmosphere. The mixture was diluted with MeOH and filtered. The filtrate was delivered to prep-HPLC (column is Waters Sunfire C18, 5 um, 30 mm ID*150 mm length. Solvent A: H₂O (0.05% FA); Solvent B: ACN (0.05% FA)) to afford 4-ethoxy-6-(1-(7-(2-(ethyl(methyl)amino)ethyl)-1-oxo-5-(pyrrolidin-1-yl)-3,4-dihydroisoquinolin-2(1H)-yl)ethyl)nicotinonitrile (207) as an oil. ¹H NMR (400 MHz, Methanol-d4) δ 8.64 (s, 1H), 7.59 (d, J=1.7 Hz, 1H), 7.27-7.19 (m, 2H), 5.94 (q, J=7.1 Hz, 1H), 4.32 (qd, J=7.0, 1.5 Hz, 2H), 3.62-3.32 (m, 8H), 3.26-3.01 (m, 4H), 3.00-2.85 (m, 5H), 2.12-1.98 (m, 4H), 1.67 (dd, J=7.2, 2.6 Hz, 3H), 1.48 (td, J=7.0, 2.2 Hz, 3H), 1.35 (td, J=7.3, 2.8 Hz, 3H). LC-MS: 476 (M+1).

Following a similar procedure to that of Example 207 (Unless otherwise specified). The following compounds were prepared from their corresponding precursors.

| Ex. | Name/Structure | ¹HNMR/LC-MS | Precursors |
|---|---|---|---|
| 208 | 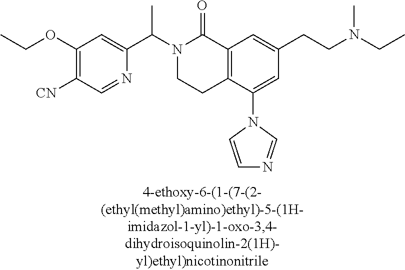 | 1H NMR (400 MHz, Methanol-d4) δ 8.64 (s, 1H), 8.49 (s, 1H), 8.07 (d, J = 1.8 Hz, 1H), 7.90 (d, J = 1.1 Hz, 1H), 7.58 (d, J = 1.8 Hz, 1H), 7.38 (d, J = 1.4 Hz, 1H), 7.26-7.19 (m, 2H), 5.99 (q, J = 7.1 Hz, 1H), 4.35 (q, J = 7.0 Hz, 2H), 3.67-3.49 (m, 2H), 3.42-3.34 (m, 2H), 3.28-3.14 (m, 4H), 2.89 (s, 3H), 2.86-2.73 (m, 2H), 1.69 (d, J = 7.1 Hz, 3H), 1.50 (t, J = 7.0 Hz, 3H), 1.36 (t, J = 7.3 Hz, 3H). LC-MS: [M + H]⁺ = 473. | pyrrolidine was replaced with 1H- imidazole |
| 209 | 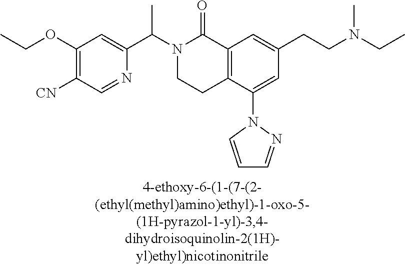 | 1H NMR (400 MHz, Methanol-d4) δ 8.64 (s, 1H), 8.56 (s, 3H), 8.04 (d, J = 1.8 Hz, 1H), 7.94 (d, J = 2.5 Hz, 1H), 7.78 (d, J = 1.9 Hz, 1H), 7.58 (d, J = 1.8 Hz, 1H), 7.22 (s, 1H), 6.58 (t, J = 2.2 Hz, 1H), 6.00 (q, J = 7.1 Hz, 1H), 4.34 (q, J = 7.1 Hz, 2H), 3.63-3.47 (m, 2H), 3.19-3.08 (m, 4H), 3.01 (q, J = 7.3 Hz, 2H), 2.90-2.82 (m, 2H), 2.71 (s, 3H), 1.68 (d, J = 7.1 Hz, 3H), 1.50 (t, J = 7.0 Hz, 3H), 1.28 (t, J = 7.2 Hz, 3H). LC-MS: [M + H]⁺ = 473. | Step 2: pyrrolidine was replaced with 1H-pyrazole |
| 210 | 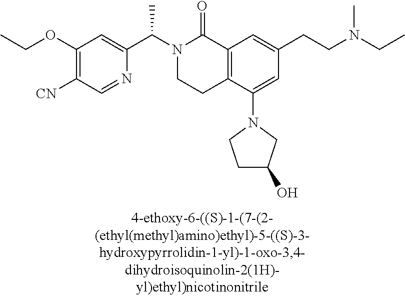 | 1H NMR (400 MHz, Methanol-d4) δ 8.60 (d, J = 10.1 Hz, 1H), 7.41 (s, 1H), 7.17 (d, J = 5.4 Hz, 1H), 7.01 (t, J = 1.6 Hz, 1H), 5.92 (h, J = 6.7 Hz, 1H), 4.48-4.41 (m, 1H), 4.32-4.24 (m, 2H), 3.59- 3.29 (m, 7H), 3.20-2.97 (m, 5H), 2.89 (d, J = 6.4 Hz, 5H), 2.19- 2.08 (m, 1H), 1.91 (d, J = 7.5 Hz, 1H), 1.63 (dd, J = 10.0, 7.1 Hz, 3H), 1.44 (t, J = 7.0 Hz, 3H), 1.32 (t, J = 7.3 Hz, 3H). LC-MS: [M + H]⁺ = 492. | Step 2: pyrrolidine was replaced with (S)-pyrrolidin-3-ol |

### Example 211: Synthesis of 5-(3,6-dimethylpyridazin-4-yl)-2-(1-(4-ethoxy-5-methylpyridin-2-yl)ethyl)-7-(2-(ethyl(methyl)amino)ethyl)-3,4-dihydroisoquinolin-1(2H)-one (211)

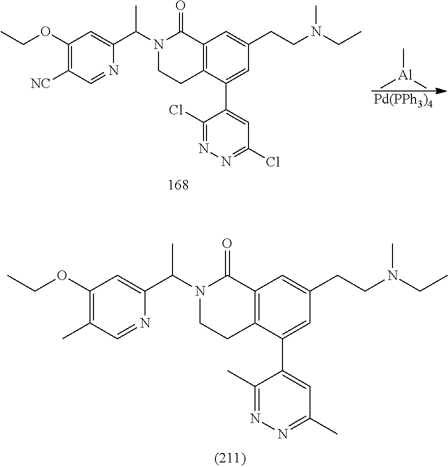

To a mixture of compound 168 (100 mg, 0.181 mmol), Pd(PPh₃)₄ (31.3 mg, 0.027 mmol) in dioxane (3 mL) was added trimethylaluminum (0.903 mL, 1M, 0.903 mmol) under N₂ atmosphere. The mixture was then stirred for 20 hrs. at 95° C. The mixture was cooled to RT and then quenched with MeOH. The mixture was filtered and the filtrate was concentrated under reduced pressure. The residue was purified by pre-HPLC to afford 5-(3,6-dimethylpyridazin-4-yl)-2-(1-(4-ethoxy-5-methylpyridin-2-yl)ethyl)-7-(2-(ethyl(methyl)amino)ethyl)-3,4-dihydroisoquinolin-1(2H)-one (211) as a yellow solid (Solvent A: H₂O (0.05% FA); Solvent B: ACN (0.05% FA); Prep Selection Valve: Prep Column 1). ¹H NMR (400 MHz, Methanol-d4) δ 8.38 (s, 2H), 8.10 (dd, J=19.1, 1.9 Hz, 2H), 7.44-7.31 (m, 2H), 6.96 (d, J=2.6 Hz, 1H), 5.99 (q, J=7.1 Hz, 1H), 4.20-4.10 (m, 2H), 3.54-3.35 (m, 3H), 3.25 (q, J=7.4 Hz, 3H), 3.19-3.11 (m, 2H), 2.91 (s, 3H), 2.67 (d, J=8.1 Hz, 3H), 2.54 (dddd, J=31.2, 16.0, 7.3, 4.7 Hz, 2H), 2.40 (d, J=20.3 Hz, 3H), 2.14 (s, 3H), 1.62 (dd, J=7.1, 1.3 Hz, 3H), 1.43 (t, J=7.0 Hz, 3H), 1.35 (t, J=7.3 Hz, 3H). LC-MS: 502 (M+1).

Following a similar procedure to that of Example 211 (Unless otherwise specified). The following compounds were prepared from their corresponding precursors:

| Ex. | Name/Structure | ¹HNMR/LC-MS | Precursors |
|---|---|---|---|
| 212 | 5-(3,6-dimethylpyridazin-4-yl)-2-((4- | ¹H NMR (400 MHz, Methanol-d₄) | Compound 168 |
| | ethoxy-5-methylpyridin-2-yl)methyl)- | δ 8.42 (s, 2H), 8.13-8.05 (m, | was replaced |
| | 7-(2-(ethyl(methyl)amino)ethyl)-3,4- | 2H), 7.44 (s, 1H), 7.39 (d, J = 2.0 | withcompound |
| | dihydroisoquinolin-1(2H)-one | Hz, 1H), 6.97 (s, 1H), 4.82 (d, J = | 189 |
| | 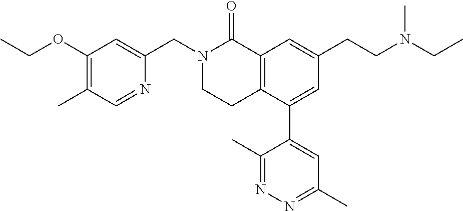 [SMILES: CCOc1cc(CN2CCc3c(cc(CCN(C)CC)cc3-c3cc(C)nnc3C)C2=O)ncc1C] | 5.0 Hz, 2H), 4.14 (q, J = 7.0 Hz, 2H), 3.60-3.52 (m, 2H), 3.44- 3.36 (m, 2H), 3.26 (t, J = 7.3 Hz, 2H), 3.21-3.12 (m, 2H), 2.91 (s, 3H), 2.78-2.56 (m, 5H), 2.42 (s, 3H), 2.15 (s, 3H), 1.43 (t, J = 7.0 Hz, 3H), 1.36 (t, J = 7.3 Hz, 3H). LC-MS: [M + H]⁺ = 488 | |

### Example 213: Synthesis of 6-((S)-1-(5-(3-(difluoromethyl)-1-methyl-1H-pyrazol-4-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile (213)

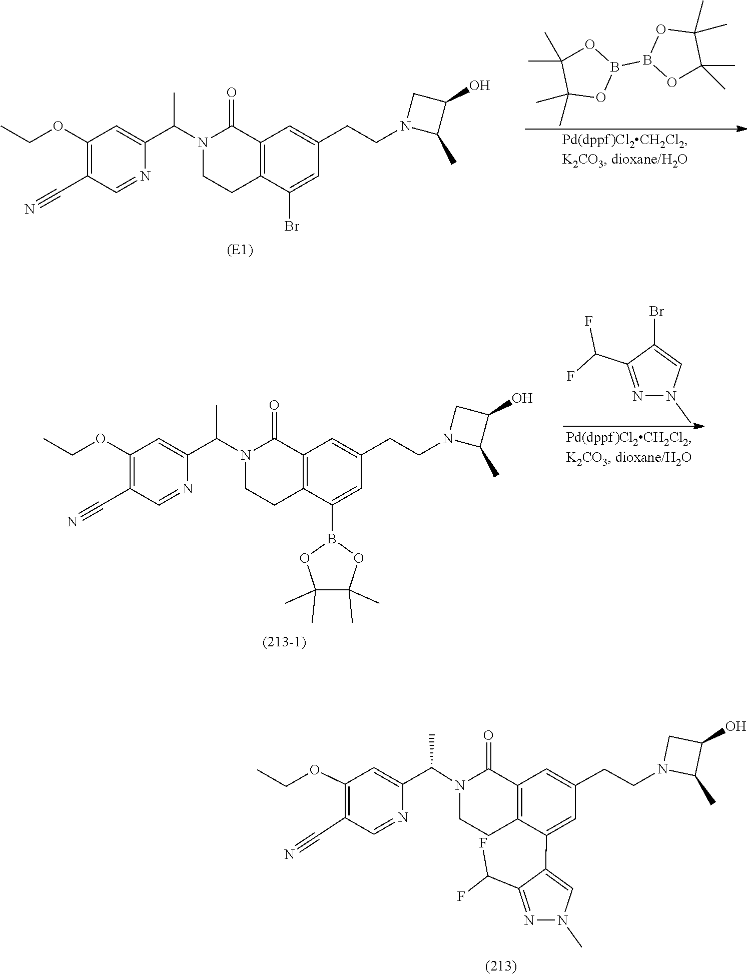

Step 1: To a solution of Intermediate (E1) (300 mg, 0.584 mmol, 1.0 eq) in dioxane (2.5 mL) was added compound 4,4,4′,4′,5,5,5′,5′-octamethyl-2,2′-bi(1,3,2-dioxaborolane) (178 mg, 0.701 mmol, 1.2 eq) and KOAc (143 mg, 1.46 mmol, 2.5 eq). Pd(dppf)Cl₂.CH₂Cl₂ (48 mg, 0.0584 mmol, 0.1 eq) was added to the mixture under N₂. The reaction mixture was stirred at 100° C. for 2 hrs under N₂. The mixture was diluted with H₂O (20 mL) and extracted with EtOAc (20 mL×3). The combined organic layers were dried over anhydrous Na₂SO₄, filtered and concentrated in vacuum to give 4-ethoxy-6-(1-(7-(2-((2R,3R)-3-hydroxy-2-methylazetidin-1-yl)ethyl)-1-oxo-5-(4,4,5,5-tetramethyl-1,3,2-dioxaborolan-2-yl)-3,4-dihydroisoquinolin-2(1H)-yl)ethyl)nicotinonitrile (213-1) (580 mg, 60% purity) as a yellow oil. LCMS: 561.6 [M+H]+

Step 2: To a solution of compound 213-1 (360 mg, 60% purity, 0.31 mmol, 1.0 eq) in dioxane (1.6 mL)/H₂O (0.4 mL) was added compound 4-bromo-3-(difluoromethyl)-1-methyl-1H-pyrazole (84 mg, 0.372 mmol, 1.2 eq) and K₂CO₃ (129 mg, 0.93 mmol, 3.0 eq). Pd(dppf)Cl₂.CH₂Cl₂ (25 mg, 0.031 mmol, 0.1 eq) was added to the reaction mixture under N₂. The mixture was stirred at 80° C. for 1.5 hrs under N₂. The mixture was diluted with H₂O (30 mL). The resulting mixture was extracted with EtOAc (20 mL×3). The combined organic layers were dried over anhydrous Na₂SO₄, filtered and concentrated. The residue was purified by column chromatography on silica gel eluting with dichloromethane:methanol (40% methanol) to give crude product (190 mg). The crude was purified by Prep-HPLC (Column: Waters Xbridge 150*25 5 u; Condition: A=water (10 mM NH4HCO3)-ACN, 25-55% of B, B=acetonitrile, flow rate: 25 ml/min, Gradient Time (min): 10 min) to give a racemic mixture as a white solid. The racemic mixture (70 mg, 0.124 mmol, 1.0 eq) was purified by Prep-SFC (column: DAICEL CHIRALCEL OD (250 mm*30 mm, 10 um); Gradient Time: 3 min; 30 min; Condition: 0.1% NH₃H₂O EtOH; Flow Rate: 70 g/min; 40% of B) to give 6-((S)-1-(5-(3-(difluoromethyl)-1-methyl-1H-pyrazol-4-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile (213) as a white solid. ¹H NMR (400 MHz, Methanol-d₄) δ ppm 8.62 (s, 1H), 7.87 (d, J=1.8 Hz, 1H), 7.67 (s, 1H), 7.33 (d, J=1.8 Hz, 1H), 7.18 (s, 1H), 6.85-6.47 (m, 1H), 5.98 (d, J=7.1 Hz, 1H), 4.34-4.26 (m, 3H), 3.96 (s, 3H), 3.57-3.49 (m, 1H), 3.47-3.40 (m, 2H), 3.26 (dd, J=1.5, 9.0 Hz, 1H), 3.21-3.16 (m, 1H), 2.91-2.82 (m, 1H), 2.81-2.69 (m, 4H), 2.67-2.60 (m, 1H), 1.65 (d, J=7.2 Hz, 3H), 1.47 (t, J=7.0 Hz, 3H), 1.19 (d, J=6.6 Hz, 3H). LC-MS: [M+H]⁺=565.3

Following a similar procedure to that of Example 213 (unless otherwise specified). The following compounds were prepared from their corresponding precursors.

| Ex. | Name/Structure | ¹HNMR/LC-MS | Precursors |
|---|---|---|---|
| 214 | 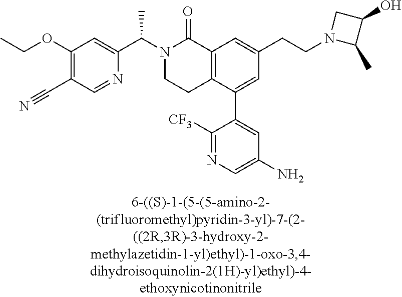 [SMILES: CCOc1cc([C@H](C)N2CCc3c(cc(CCN4C[C@@H](O)[C@H]4C)cc3-c3cc(N)cnc3C(F)(F)F)C2=O)ncc1C#N] | ¹H NMR (400 MHz, Methanol-d₄) δ ppm 8.51 (d, J = 2.6 Hz, 1H), 7.95-7.88 (m, 1H), 7.82 (s, 1H), 7.15 (s, 1H), 7.08 (d, J = 8.4 Hz, 1H), 6.71 (dd, J = 2.4, 14.8 Hz, 1H), 5.93-5.80 (m, 1H), 4.31- 4.14 (m, 3H), 3.54 (br s, 1H), 3.46 (ddd, J = 4.6, 8.1, 12.6 Hz, 1H), 3.39 (br t, J = 6.4 Hz, 1H), 3.34- 3.25 (m, 3H), 2.94-2.83 (m, 1H), 2.76-2.62 (m, 3H), 2.61-2.37 (m, 2H), 1.54 (dd, J = 2.3, 7.1 Hz, 3H), 1.37 (dt, J = 4.1, 7.0 Hz, 3H), 1.15 (dd, J = 2.8, 6.7 Hz, 3H). LC-MS: [M + H]⁺ = 595.3 | 4-bromo-3- (difluoromethyl)- 1-methyl-1H- pyrazole was replaced with 5- bromo-6- (trifluoromethyl) pyridin-3-amine |
| 215 | 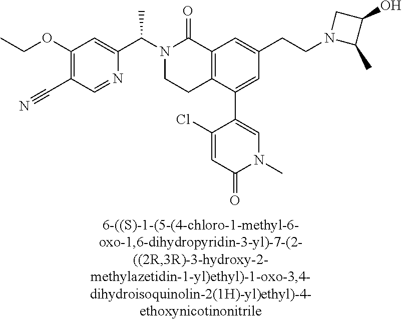 [SMILES: CCOc1cc([C@H](C)N2CCc3c(cc(CCN4C[C@@H](O)[C@H]4C)cc3-c3cn(C)c(=O)cc3Cl)C2=O)ncc1C#N] | ¹H NMR (400 MHz, Methanol-d₄) δ ppm 8.62 (d, J = 7.8 Hz, 1H), 7.93 (d, J = 1.7 Hz, 1H), 7.71 (d, J = 15.2 Hz, 1H), 7.30 (d, J = 1.8 Hz, 1H), 7.19 (d, J = 2.2 Hz, 1H), 6.74 (d, J = 5.5 Hz, 1H), 5.98 (dd, J = 2.5, 7.2 Hz, 1H), 4.37-4.26 (m, 3H), 3.58 (d, J = 6.1 Hz, 3H), 3.56-3.42 (m, 3H), 3.27- 3.18 (m, 2H), 2.89 (br d, J = 4.0 Hz, 1H), 2.81-2.61 (m, 5H), 1.65 (dd, J = 4.4, 7.1 Hz, 3H), 1.47 (t, J = 7.0 Hz, 3H), 1.21 (dd, J = 2.6, 6.6 Hz, 3H). LC-MS: [M + H]⁺ = 576.2 | 4-bromo-3- (difluoromethyl)- 1-methyl-1H- pyrazole was replaced with 5- bromo-4-chloro-1- methylpyridin- 2(1H)-one |
| 216 | 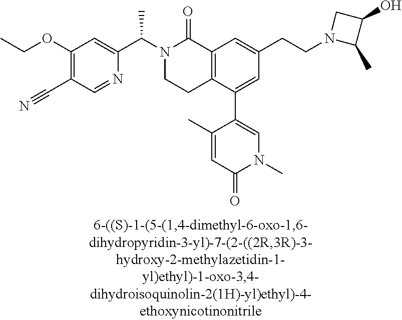 [SMILES: CCOc1cc([C@H](C)N2CCc3c(cc(CCN4C[C@@H](O)[C@H]4C)cc3-c3cn(C)c(=O)cc3C)C2=O)ncc1C#N] | ¹H NMR (400 MHz, Methanol-d₄) δ ppm 8.61 (s, 1H), 7.91 (d, J = 1.3 Hz, 1H), 7.46 (d, J = 14.7 Hz, 1H), 7.26 (d, J = 1.8 Hz, 1H), 7.18 (d, J = 2.8 Hz, 1H), 6.50 (d, J = 5.5 Hz, 1H), 5.98 (dq, J = 4.0, 7.0 Hz, 1H), 4.36-4.27 (m, 3H), 3.60-3.38 (m, 6H), 3.28-3.24 (m, 1H), 3.22- 3.16 (m, 1H), 2.91-2.57 (m, 6H), 1.94 (d, J = 18.2 Hz, 3H), 1.65 (d, J = 7.2 Hz, 3H), 1.47 (t, J = 7.0 Hz, 3H), 1.19 (d, J = 6.5 Hz, 3H). LC-MS: [M + H]⁺ = 556 | 4-bromo-3- (difluoromethyl)- 1-methyl-1H- pyrazole was replaced with 5- bromo-1,4- dimethylpyridin- 2(1H)-one |
| 217 | 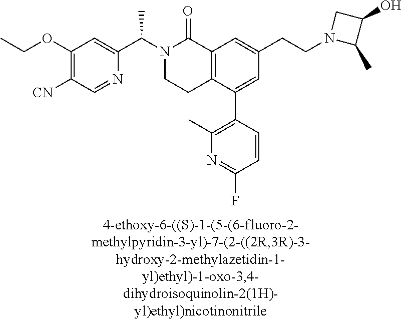 | ¹H NMR (400 MHz, Chloroform- d) δ 12.57 (dd, J = 3.2, 1.3 Hz, 1H), 12.00 (s, 0H), 11.89 (d, J = I.6 Hz, 1H), 11.64 (dtd, J = 13.2, 8.2, 1.3 Hz, 1H), 11.22 (d, J = 1.7 Hz, 1H), 11.14 (dd, J = 3.6, 1.2 Hz, 1H), 10.98-10.88 (m, 1H), 10.00-9.87 (m, 1H), 8.35-8.22 (m, 3H), 7.57-7.35 (m, 3H), 7.24 (d, J = 11.1 Hz, 2H), 6.88 (dd, J = 11.0, 5.0 Hz, 1H), 6.80-6.62 (m, 3H), 6.61-6.45 (m, 2H), 6.18 (dd, J = 18.0, 1.3 Hz, 3H), 5.61 (dt, J = 7.2, 1.7 Hz, 3H), 5.43 (td, J = 7.0, 1.3 Hz, 3H), 5.17 (dt, J = 6.7, 1.3 Hz, 3H). LC-MS: [M + H]⁺ = 544 | 4-bromo-3- (difluoromethyl)- 1-methyl-1H- pyrazole was replaced with 3- bromo-6-fluoro-2- methylpyridine |
| 218 | 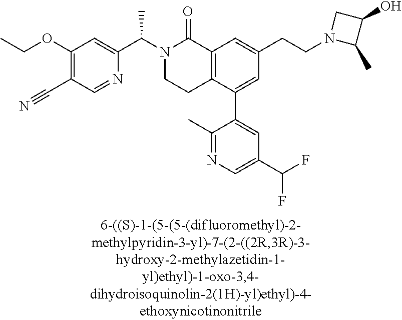 [SMILES: CCOc1cc([C@H](C)N2CCc3c(cc(CCN4C[C@@H](O)[C@H]4C)cc3-c3cc(C(F)F)cnc3C)C2=O)ncc1C#N] | ¹H NMR (400 MHz, Methanol-d₄) δ 8.68-8.57 (m, 2H), 7.96 (s, 1H), 7.75 (br d, J = 12.4 Hz, 1H), 7.28 (d, J = 1.2 Hz, 1H), 7.19 (d, J = 5.0 Hz, 1H), 7.08-6.75 (m, 1H), 5.98 (br dd, J = 2.4, 7.2 Hz, 1H), 4.31 (dq, J = 4.0, 6.8 Hz, 3H), 3.62-3.51 (m, 1H), 3.50-3.39 (m, 2H), 3.29-3.15 (m, 2H), 2.88 (br dd, J = 5.2, 9.0 Hz, 1H), 2.82-2.73 (m, 2H), 2.72-2.48 (m, 3H), 2.35 (d, J = 18.4 Hz, 3H), 1.65 (dd, J = 2.4, 7.2 Hz, 3H), 1.47 (dt, J = 1.2, 7.0 Hz, 3H), 1.19 (d, J = 6.4 Hz, 3H) LC-MS: [M + H]⁺ = 576 | 4-bromo-3- (difluoromethyl)- 1-methyl-1H- pyrazole was replaced with 3- bromo-5- (difluoromethyl)-2- methylpyridine |
| 219 | 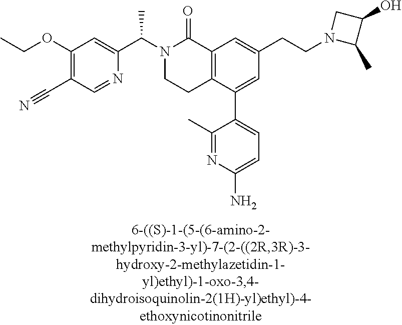 [SMILES: CCOc1cc([C@H](C)N2CCc3c(cc(CCN4C[C@@H](O)[C@H]4C)cc3-c3ccc(N)nc3C)C2=O)ncc1C#N] | ¹H NMR (400 MHz, Methanol-d₄) δ 8.61 (d, J = 3.2 Hz, 1H), 7.87 (d, J = 1.6 Hz, 1H), 7.22-7.14 (m, 3H), 6.48 (dd, J = 5.6, 8.4 Hz, 1H), 6.03-5.90 (m, 1H), 4.36- 4.25 (m, 3H), 3.57-3.49 (m, 1H), 3.43 (q, J = 6.4 Hz, 2H), 3.29-3.24 (m, 1H), 3.23-3.16 (m, 1H), 2.92-2.83 (m, 1H), 2.79- 2.70 (m, 2H), 2.69-2.57 (m, 3H), 2.07 (d, J = 18.8 Hz, 3H), 1.65 (dd, J = 2.4, 7.2 Hz, 3H), 1.47 (t, J = 7.2 Hz, 3H), 1.19 (dd, J = 1.2, 6.4 Hz, 3H). LC-MS: [M + H]⁺ = 541. | 4-bromo-3- (difluoromethyl)- 1-methyl-1H- pyrazole was replaced with 5- bromo-6- methylpyridin-2- amine |
| 220 | 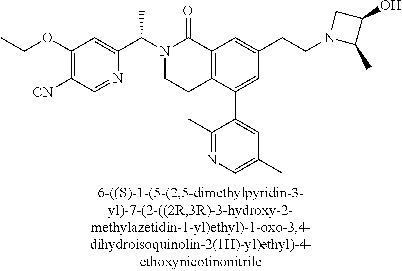 | ¹H NMR (400 MHz, Methanol-d₄) δ 8.61 (d, J = 3.0 Hz, 1H), 8.29 (d, J = 2.2 Hz, 1H), 7.92 (d, J = 1.9 Hz, 1H), 7.42 (dd, J = 13.4, 2.2 Hz, 1H), 7.21 (dd, J = 21.8, 2.7 Hz, 2H), 5.98 (qd, J = 7.1, 3.2 Hz, 1H), 4.36-4.26 (m, 3H), 3.58- 3.35 (m, 3H), 3.28-3.12 (m, 2H), 2.87 (tdd, J = 10.5, 5.4, 1.5 Hz, 1H), 2.82-2.71 (m, 2H), 2.70- 2.47 (m, 3H), 2.35 (d, J = 5.9 Hz, 3H), 2.22 (d, J = 18.1 Hz, 3H), 1.64 (dd, J = 7.1, 2.4 Hz, 3H), 1.47 (t, J = 7.0 Hz, 3H), 1.19 (dd, J = 6.5, 1.4 Hz, 3H). LC-MS: [M + H]⁺ = 540 | 4-bromo-3- (difluoromethyl)- 1-methyl-1H- pyrazole was replaced with 3- bromo-2,5- dimethylpyridine |
| 221 | 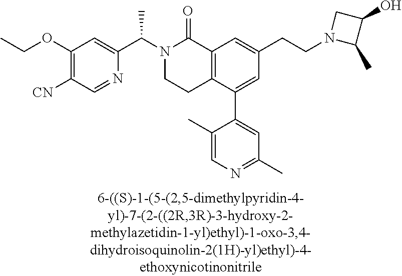 | ¹H NMR (400 MHz, Methanol-d₄) δ 8.61 (s, 1H), 8.53 (s, 1H), 8.35 (d, J = 4.1 Hz, 1H), 7.96 (d, J = 1.9 Hz, 1H), 7.22 (dd, J = 27.6, 2.6 Hz, 2H), 7.07 (d, J = 12.3 Hz, 1H), 6.05-5.91 (m, 1H), 4.47 (q, J = 5.3 Hz, 1H), 4.31 (dt, J = 7.9, 6.2 Hz, 2H), 4.11 (d, J = 18.9 Hz, 1H), 3.81 (s, 1H), 3.69-3.41 (m, 3H), 3.28-3.01 (m, 2H), 2.90 (q, J = 9.8, 9.3 Hz, 2H), 2.72-2.46 (m, 5H), 2.04 (d, J = 16.7 Hz, 3H), 1.65 (d, J = 7.1 Hz, 3H), 1.47 (t, J = 7.0 Hz, 3H), 1.38 (d, J = 6.7 Hz, 3H). LC-MS: [M + H]⁺ = 540. | 4-bromo-3- (difluoromethyl)- 1-methyl-1H- pyrazole was replaced with 4- bromo-2,5- dimethylpyridine |
| 222 | 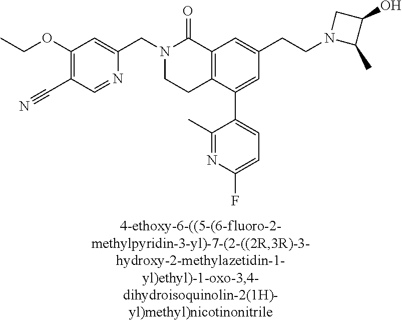 [SMILES: CCOc1cc(CN2CCc3c(cc(CCN4C[C@@H](O)[C@H]4C)cc3-c3ccc(F)nc3C)C2=O)ncc1C#N] | ¹H NMR (400 MHz, Methanol-d₄) δ 8.61 (s, 1H), 8.47 (s, 1H), 8.01 (d, J = 1.6 Hz, 1H), 7.73 (t, J = 8.0 Hz, 1H), 7.40 (d, J = 1.6 Hz, 1H), 7.21 (s, 1H), 7.01 (dd, J = 2.4, 8.2 Hz, 1H), 4.91 (d, J = 1.2 Hz, 2H), 4.62-4.52 (m, 2H), 4.33 (q, J = 7.0 Hz, 2H), 4.18 (br, 1H), 3.93 (br, 1H), 3.71-3.64 (m, 2H), 3.54-3.44 (m, 1H), 3.43- 3.35 (m, 1H), 3.11-2.96 (m, 2H), 2.81-2.65 (m, 2H), 2.26 (s, 3H), 1.54-1.46 (m, 6H). LC-MS: [M + H]⁺ = 530. | 4-bromo-3- (difluoromethyl)- 1-methyl-1H- pyrazole was replaced with 3- bromo-6-fluoro-2- methylpyridine |
| 223 | 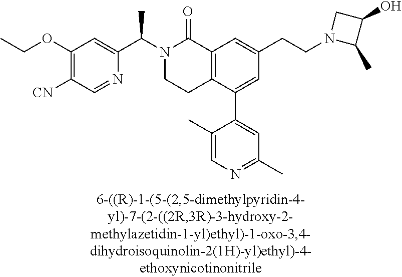 | ¹H NMR (400 MHz, Methanol-d₄) δ 8.60 (s, 1H), 8.33 (d, J = 4.3 Hz, 1H), 7.93 (d, J = 1.9 Hz, 1H), 7.26- 7.13 (m, 2H), 7.06 (d, J = 11.7 Hz, 1H), 5.98 (qd, J = 7.1, 2.7 Hz, 1H), 4.31 (ddt, J = 10.0, 7.0, 3.8 Hz, 3H), 3.61-3.36 (m, 3H), 3.28- 3.13 (m, 2H), 2.96-2.58 (m, 5H), 2.51 (d, J = 5.4 Hz, 4H), 2.04 (d, J = 17.0 Hz, 3H), 1.64 (d, J = 7.1 Hz, 3H), 1.47 (t, J = 7.0 Hz, 3H), 1.19 (dd, J = 6.6, 1.3 Hz, 3H). LC-MS: [M + H]⁺ = 540. | 4-bromo-3- (difluoromethyl)- 1-methyl-1H- pyrazole was replaced with 4- bromo-2,5- dimethylpyridine |
| 224 | 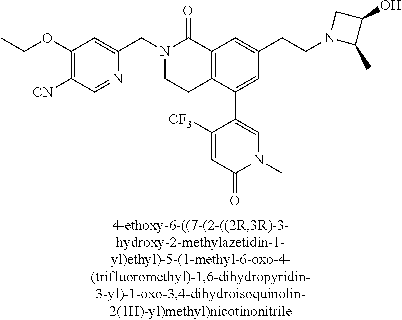 | ¹H NMR (400 MHz, CDCl3) δ 8.59 (s, 1H), 8.01 (s, 1H), 7.23 (d, J = 4.6 Hz, 1H), 7.22-7.19 (m, 1H), 7.04 (s, 1H), 7.01 (s, 1H), 4.84 (d, J = 3.6 Hz, 2H), 4.36- 4.29 (m, 1H), 4.24 (q, J = 7.0 Hz, 2H), 3.67-3.59 (m, 5H), 3.46- 3.34 (m, 2H), 3.24-3.13 (m, 1H), 2.97-2.88 (m, 1H), 2.84-2.73 (m, 3H), 2.71-2.59 (m, 2H), 2.20 (s, 1H), 1.51 (t, J = 7.0 Hz, 3H), 1.24 (d, J = 6.5 Hz, 3H). LC-MS: [M + H]⁺ = 596. | 4-bromo-3- (difluoromethyl)- 1-methyl-1H- pyrazole was replaced with 5- bromo-1-methyl-4- (trifluoromethyl) pyridin-2(1H)-one |

### Example 225: Synthesis of 4-ethoxy-6-((S)-1-(5-(6-fluoro-2-methylpyridin-3-yl)-7-(2-((2R, 3R)-3-hydroxy-2-methylpyrrolidin-1-yl)ethyl)-1-oxo-3,4-dihydroisoquinolin-2(1H)-yl)ethyl)nicotinonitrile (225)

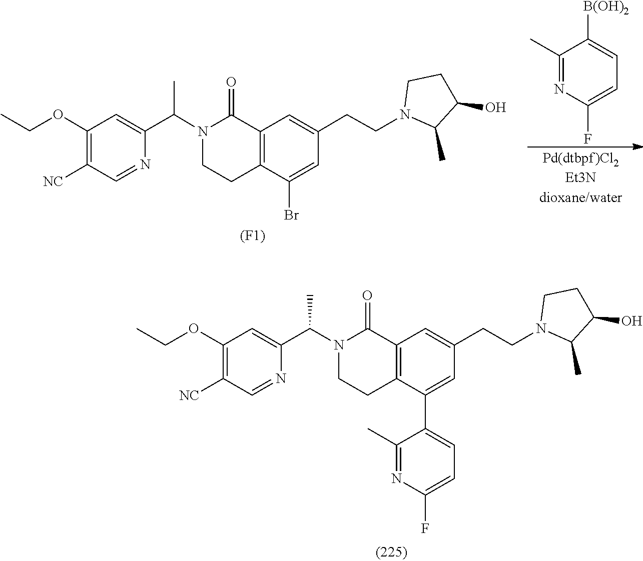 [SMILES: CCOc1cc(C(C)N2CCc3c(Br)cc(CCN4CC[C@@H](O)[C@H]4C)cc3C2=O)ncc1C#N ; Cc1nc(F)ccc1B(O)O ; "(F1)" ; CCOc1cc([C@H](C)N2CCc3c(cc(CCN4CC[C@@H](O)[C@H]4C)cc3-c3ccc(F)nc3C)C2=O)ncc1C#N]

To a solution of intermediate (F1) (150 mg, 0.29 mmol, 1 equiv, crude) in dioxane (4 mL) and H₂O (1 mL) was added (6-fluoro-2-methylpyridin-3-yl) boronic acid (66 mg, 0.43 mmol, 1.5 equiv), Et₃N (86 mg, 0.86 mmol, 3 equiv), Pd(dtbpf)Cl₂ (19 mg, 0.029 mmol, 0.1 equiv) at 25° C. under nitrogen atmosphere. The reaction mixture was stirred at 40° C. for 2 hrs. MeCN (10 mL) was added. The resulting mixture was stirred with with thiourea resin (1 g) at 25° C. for 12 hrs. The resulting mixture was filtered and washed with MeCN (10 mL×3). The combined organic phase was concentrated under reduced pressure. The residue was purified by Prep-HPLC (Shim-pack C18 150*25*10 um, Gradient: 15%-45% of B. A: water (0.225% FA), B: Acetonitrile, flowrate: 25 mL/min). The resulting mixture was concentrated under reduced pressure and adjust pH 8 by sat NaHCO₃ and extracted with ethyl acetate (10 mL×3). The combined organic phase was concentrated under reduced pressure to afford compound racemate compound (100 mg) as a colorless oil. The residue (100 mg, 0.18 mmol, 1 eq) was separated by SFC (column: DAICEL CHIRALCEL OD (250 mm*30 mm, 10 um), condition: 0.1% NH₃H₂O MeOH, flow rate: 70 g/min) to afford 4-ethoxy-6-((S)-1-(5-(6-fluoro-2-methylpyridin-3-yl)-7-(2-((2R, 3R)-3-hydroxy-2-methylpyrrolidin-1-yl)ethyl)-1-oxo-3,4-dihydroisoquinolin-2(1H)-yl)ethyl)nicotinonitrile (225) as a yellow solid. ¹H NMR (400 MHz, Methanol-d₄) δ 8.63 (d, J=2.8 Hz, 1H), 7.98 (s, 1H), 7.78-7.65 (d, 1H), 7.30 (d, J=1.6 Hz, 1H), 7.20 (d, J=3.2 Hz, 1H), 7.03-6.95 (m, 1H), 7.02-6.95 (m, 1H), 6.00 (m, 1H), 4.33 (m, 2H), 4.16 (br, 1H), 3.64-3.41 (m, 2H), 3.19-3.06 (m, 1H), 3.02-2.87 (m, 2H), 2.71-2.52 (m, 2H), 2.46 (br, 2H), 2.32 (br, 1H), 2.24 (d, J=18.4 Hz, 4H), 1.80-1.70 (s, 1H), 1.67 (dd, J=1.6, 7.3 Hz, 3H), 1.49 (t, J=7.0 Hz, 3H), 1.16 (d, J=6.3 Hz, 3H). LC-MS: [M+H]⁺=558.3

Following a similar procedure to that of Example 225 (unless otherwise specified). The following compounds were prepared from their corresponding precursors.

| Ex. | Name/Structure | ¹HNMR/LC-MS | Precursors |
|---|---|---|---|
| 226 | 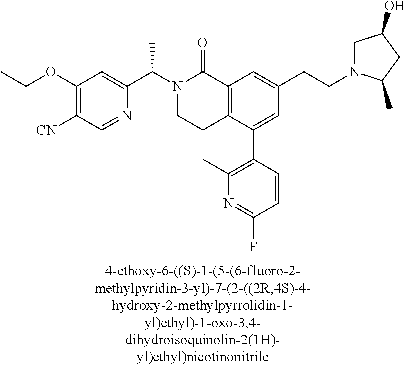 | ¹H NMR (400 MHz, Methanol-d₄) δ 8.61 (d, J = 3.2 Hz, 1H), 8.51 (br s, 1H), 7.99 (d, J = 1.6 Hz, 1H), 7.70 (td, J = 8.2, 14.4 Hz, 1H), 7.34 (d, J = 1.6 Hz, 1H), 7.20 (d, J = 3.2 Hz, 1H), 7.02-6.95 (m, 1H), 6.00 (dq, J = 2.6, 7.2 Hz, 1H), 4.47 (br s, 1H), 4.32 (dq, J = 2.8, 7.0 Hz, 2H), 3.65-3.40 (m, 4H), 3.13- 3.01 (m, 4H), 2.70-2.50 (m, 4H), 2.23 (d, J = 18.0 Hz, 3H), 1.74- 1.57 (m, 5H), 1.51-1.42 (m, 7H). LC-MS: [M + H]⁺ = 558. | (6-fluoro-2- methylpyridin-3-yl) boronic acid was replaced with 3- bromo-6-fluoro-2- methylpyridine and (3S,5R)-5- methylpyrrolidin-3- ol |
| 227 | 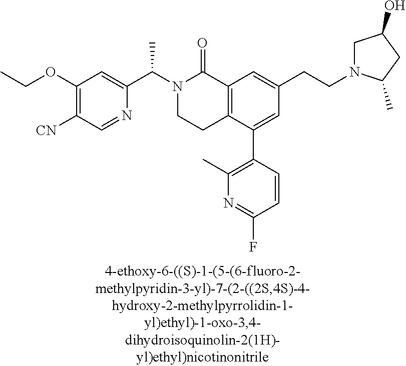 | ¹H NMR (400 MHz, Methanol-d₄) δ 8.61 (d, J = 3.2 Hz, 1H), 8.01 (s, 1H), 7.70 (td, J = 8.2, 10.8 Hz, 1H), 7.38 (d, J = 1.6 Hz, 1H), 7.20 (d, J = 3.6 Hz, 1H), 6.98 (ddd, J = 2.6, 5.6, 8.0 Hz, 1H), 6.02-5.93 (m, 1H), 4.36-4.28 (m, 2H), 3.96- 3.79 (m, 2H), 3.74-3.36 (m, 7H), 3.21-3.13 (m, 3H), 2.71-2.52 (m, 3H), 2.28-2.18 (m, 5H), 1.99- 1.85 (m, 2H), 1.66 (dd, J = 2.0, 7.2 Hz, 4H), 1.48 (br d, J = 7.0 Hz, 6H), 1.29 (s, 4H). LC-MS: [M + H]⁺ = 558. | (6-fluoro-2- methylpyridin-3-yl) boronic acid was replaced with 3- bromo-6-fluoro-2- methylpyridine and (3S,5S)-5- methylpyrrolidin- 3-ol |

### Example 228: Synthesis of (S)-4-ethoxy-6-(1-(5-(5-fluoro-2-(trifluoromethyl)pyridin-3-yl)-7-(2-((2-hydroxyethyl)(methyl)amino)ethyl)-1-oxo-3,4-dihydroisoquinolin-2(1H)-yl)ethyl)nicotinonitrile (228)

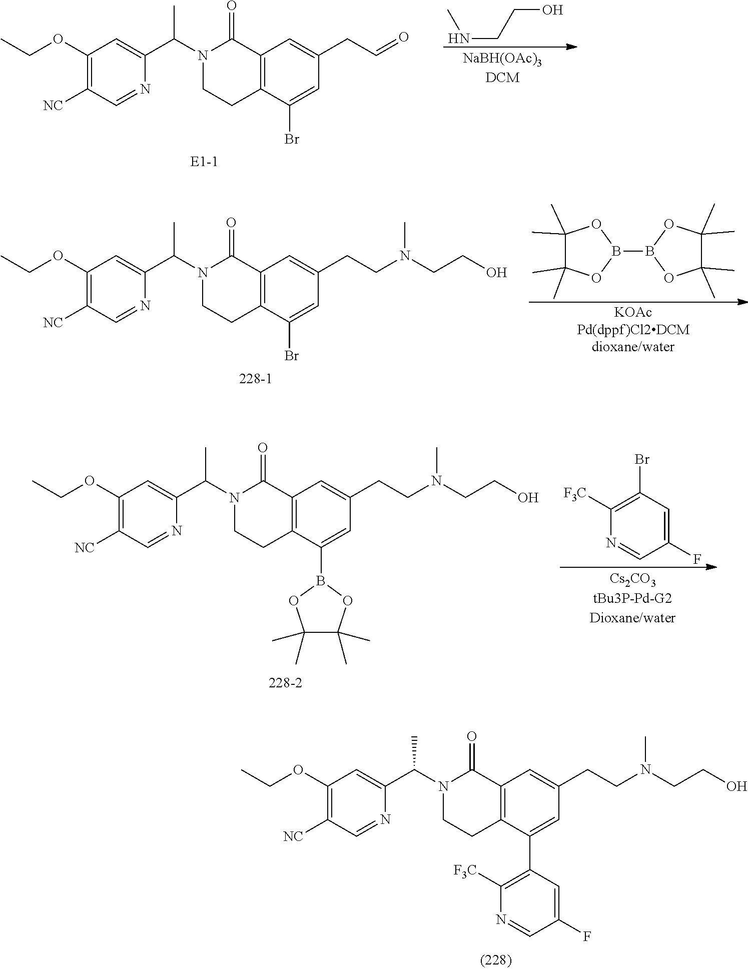

Step 1: A mixture of compound E1-1 (920 mg, 2.08 mmol, 1.0 eq), 2-(methylamino)ethan-1-ol (469 mg, 6.2 mmol, 3.0 eq) in DCM (50 mL) was stirred at 25° C. for 0.5 hrs. NaBH(OAc)₃ (1.1 g, 5.19 mmol, 2.5 eq) was added to the mixture and the mixture was stirred at 25° C. for 16 hrs. The mixture was diluted with DCM (300 mL). The resulting solution washed with water (150 mL) and aq.NaHCO₃ solution (150 mL) sequentially. The organic layer was dried over Na₂SO₄, filtered and concentrated. The residue was purified by Prep-HPLC (column: phenomenex luna C18 250*50*10 um; Gradient: 20-50% of B, A=0.225% FA in water, B=acetonitrile, flow rate: 100 mL/min). The elutent was adjusted to pH 8 by NaHCO₃ solid. The resulting solution was extracted with EtOAc (300 mL×2). The organic layer was dried over Na₂SO₄, filtered and concentrated to afford 6-(1-(5-bromo-7-(2-((2-hydroxyethyl)(methyl)amino)ethyl)-1-oxo-3,4-dihydroisoquinolin-2(1H)-yl)ethyl)-4-ethoxynicotinonitrile 228-1 (310 mg, 29% yield) as a yellow gum. LCMS: 503.2 [M+H]⁺

Step 2: To a solution of compound 228-1 (470 mg, 0.94 mmol, 1.0 eq.), 4,4,4′,4′,5,5,5′,5′-octamethyl-2,2′-bi(1,3,2-dioxaborolane) (286 mg, 1.1 mmol, 1.2 eq.), KOAc (276 mg, 2.8 mmol, 3.0 eq) in dioxane (5 mL) was added Pd(dppf)Cl₂-DCM (77 mg, 0.094 mmol, 0.1 eq) at 25° C. The mixture was stirred at 80° C. for 3 hrs. The mixture was diluted with water (200 mL). The resulting solution was extracted with ethyl acetate (200 mL×3). The organic layer was dried over Na₂SO₄, filtered and concentrated to afford 4-ethoxy-6-(1-(7-(2-((2-hydroxyethyl)(methyl)amino)ethyl)-1-oxo-5-(4,4,5,5-tetramethyl-1,3,2-dioxaborolan-2-yl)-3,4-dihydroisoquinolin-2(1H)-yl)ethyl)nicotinonitrile (228-2) (900 mg, crude) as oil, which was used directly.

Step 3: To a solution of compound 228-2 (720 mg, 60% purity, 0.8 mmol, 1.0 eq), 3-bromo-5-fluoro-2-(trifluoromethyl)pyridine (320 mg, 1.28 mmol, 1.6 eq) in dioxane/H₂O (12 mL/4 mL) was added Cs₂CO₃ (480 mg, 1.44 mmol, 1.8 eq) and tBu₃P—Pd-G₂ (40 mg, 0.078 mmol, 0.1 eq) at 25° C. The mixture was stirred at 80° C. for 2 hrs. The mixture was combined with another batch (from 180 mg of compound 228-2). The mixture was diluted with water (200 mL). The resulting solution was extracted with ethyl acetate (200 mL×2). The organic layer was dried over Na₂SO₄, filtered and concentrated. The residue was purified by Prep-HPLC (column: phenomenex luna C18 250*50*10 um; Gradient: 22-52% of B, A=0.1% TFA in water, B=acetonitrile, flow rate: 60 mL/min) to afford the crude racemate product (100 mg, yellow oil), which was purified by Prep-SEC (column: REGID(S,S) WHELK-01 (250 mm*50 mm, 10 um); Gradient Time: 4 min; 110 min; Condition: 0.1% NH₃H₂O MeOH; Flow Rate: 70 g/min; 45% of B) to (S)-4-ethoxy-6-(1-(5-(5-fluoro-2-(trifluoromethyl)pyridin-3-yl)-7-(2-((2-hydroxyethyl)(methyl)amino)ethyl)-1-oxo-3,4-dihydroisoquinolin-2(1H)-yl)ethyl)nicotinonitrile (228) as an off-white solid. ¹H NMR (400 MHz, Methanol-d₄) δ ppm 8.57 (t, J=2.3 Hz, 1H), 8.51 (s, 1H), 7.89 (d, J=1.6 Hz, 1H), 7.67-7.57 (s, 1H), 7.22 (s, 1H), 7.08 (d, J=4.0 Hz, 1H), 5.88 (quin, J=6.7 Hz, 1H), 4.21 (dq, J=2.8, 6.9 Hz, 2H), 3.60 (t, J=5.9 Hz, 2H), 3.49 (ddd, J=4.5, 8.1, 12.7 Hz, 1H), 3.43-3.37 (m, 1H), 3.36-3.27 (m, 1H), 3.36-3.27 (m, 1H), 2.91-2.82 (m, 2H), 2.78 (br d, J=5.8 Hz, 2H), 2.67 (br s, 2H), 2.63-2.51 (m, 1H), 2.47-2.30 (m, 4H), 1.55 (dd, J=2.4, 7.2 Hz, 3H), 1.37 (dt, J=3.2, 7.0 Hz, 3H). LC-MS: [M+H]⁺=586.

Following a similar procedure to that of Example 228 (unless otherwise specified). The following compounds were prepared from their corresponding precursors.

| Ex. | Name/Structure | ¹HNMR/LC-MS | Precursors |
|---|---|---|---|
| 229 | 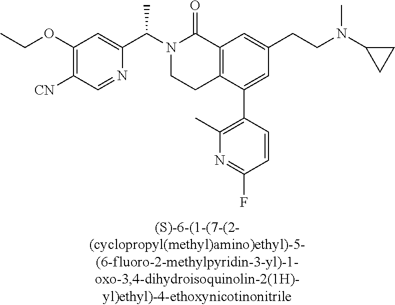 | ¹H NMR (400 MHz, Methanol-d₄) δ ppm 8.60 (d, J = 3.2 Hz, 1H), 7.94 (d, J = 1.5 Hz, 1H), 7.68 (td, J = 8.1, 13.0 Hz, 1H), 7.25 (d, J = 1.5 Hz, 1H), 7.19 (d, J = 3.2 Hz, 1H), 7.01-6.92 (m, 1H), 5.98 (dq, J = 2.9, 7.0 Hz, 1H), 4.31 (dq, J = 2.8, 7.0 Hz, 2H), 3.61-3.39 (m, 2H), 2.98-2.77 (m, 4H), 2.69- 2.48 (m, 2H), 2.42 (s, 3H), 2.22 (d, J = 17.9 Hz, 3H), 1.77 (tt, J = 3.6, 6.8 Hz, 1H), 1.65 (dd, J = 1.8, 7.1 Hz, 3H), 1.47 (t, J = 7.0 Hz, 3H), 0.58-0.47 (m, 2H), 0.44-0.35 (m, 2H). LC-MS: [M + H]⁺ = 528.3 | Step 1: 2- (methylamino) ethan-1-ol was replaced with N- methylcyclo- propanamine Step 3: 3-bromo-5-fluoro- 2-(trifluoromethyl) pyridine was replaced with 3-bromo-6-fluoro- 2-methylpyridine |
| 230 | 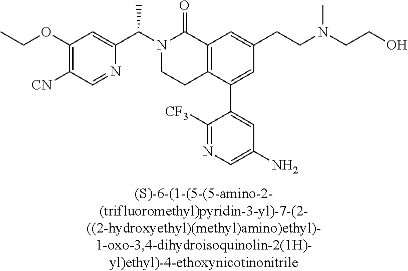 | ¹H NMR (400 MHz, Methanol-d₄) δ ppm 8.61 (d, J = 2.4 Hz, 1H), 8.00 (t, J = 2.0 Hz, 1H), 7.93 (s, 1H), 7.25 (s, 1H), 7.18 (d, J = 8.6 Hz, 1H), 6.82 (dd, J = 2.4, 14.8 Hz, 1H), 6.03-5.92 (m, 1H), 4.31 (quin, J = 6.5 Hz, 2H), 3.68 (t, J = 5.9 Hz, 2H), 3.57 (ddd, J = 4.9, 8.1, 12.8 Hz, 1H), 3.49 (br t, J = 6.4 Hz, 1H), 3.39 (ddd, J = 4.9, 7.8, 12.7 Hz, 1H), 2.98-2.86 (m, 2H), 2.86-2.76 (m, 2H), 2.75-2.60 (m, 3H), 2.60-2.48 (m, 1H), 2.44 (s, 3H), 1.65 (dd, J = 2.3, 7.1 Hz, 3H), 1.47 (dt, J = 4.4, 7.0 Hz, 3H). LC-MS: [M + H]⁺ = 583 | Step 1: 2-(methylamino) ethan-1-ol was replaced with 2- (methylamino) ethan-1-ol Step 3: 3-bromo-5-fluoro- 2-(trifluoromethyl) pyridine was replaced with 5-bromo-6- (trifluoromethyl) pyridin-3-amine |
| 231 | 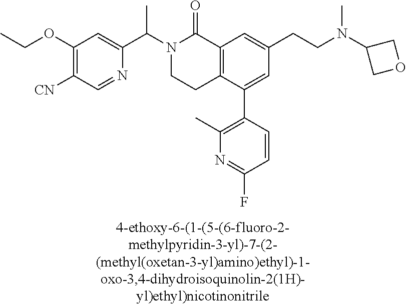 | ¹H NMR (400 MHz, Methanol-d₄) δ ppm 8.61 (d, J = 2.7 Hz, 1H), 7.93 (d, J = 1.8 Hz, 1H), 7.68 (dt, J = 12.6, 8.1 Hz, 1H), 7.27 (d, J = 1.9 Hz, 1H), 7.18 (d, J = 3.4 Hz, 1H), 6.97 (ddd, J = 8.4, 5.9, 2.6 Hz, 1H), 5.98 (qd, J = 7.1, 3.0 Hz, 1H), 4.64 (t, J = 6.7 Hz, 2H), 4.53 (t, J = 6.3 Hz, 2H), 4.35-4.21 (m, 2H), 3.65 (p, J = 6.5 Hz, 1H), 3.58-3.47 (m, 1H), 3.47-3.36 (m, 1H), 2.90-2.78 (m, 2H), 2.63- 2.47 (m, 4H), 2.28-2.15 (m, 6H), 1.64 (dd, J = 7.1, 1.8 Hz, 3H), 1.47 (t, J = 7.0 Hz, 3H). LC-MS: [M + H]⁺ = 544.3 | Step 1: 2-(methylamino) ethan-1-ol was replaced with N- methyloxetan-3- amine Step 3: 3-bromo-5-fluoro- 2- (trifluoromethyl) pyridine was replaced with 3-bromo-6-fluoro- 2-methylpyridine |
| 232 | 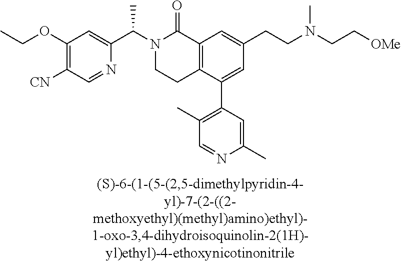 | ¹H NMR (400 MHz, Methanol-d₄) δ ppm 8.61 (s, 1H), 8.44 (s, 1H), 8.35 (d, J = 4.0 Hz, 1H), 7.98 (d, J = 1.7 Hz, 1H), 7.28 (d, J = 1.7 Hz, 1H), 7.19 (d, J = 3.1 Hz, 1H), 7.07 (d, J = 11.2 Hz, 1H), 5.97 (dd, J = 7.1, 2.6 Hz, 1H), 4.31 (q, J = 7.0 Hz, 2H), 3.72-3.63 (m, 2H), 3.59-3.45 (m, 2H), 3.40 (s, 3H), 3.26 (d, J = 5.6 Hz, 4H), 3.16-3.05 (m, 2H), 2.82 (s, 3H), 2.68-2.59 (m, 1H), 2.56 (d, J = 3.6 Hz, 1H), 2.51 (d, J = 5.0 Hz, 3H), 2.05 (d, J = 16.1 Hz, 3H), 1.65 (d, J = 7.1 Hz, 3H), 1.47 (t, J = 7.0 Hz, 3H). LC-MS: [M + H]⁺ = 542 | Step 1: 2-(methylamino) ethan-1-ol was replaced with 2- methoxy-N- methylethan-1- amine Step 3: 3-bromo-5-fluoro- 2-(trifluoromethyl) pyridine was replaced with 4-bromo-2,5- dimethylpyridine |
| 233 | 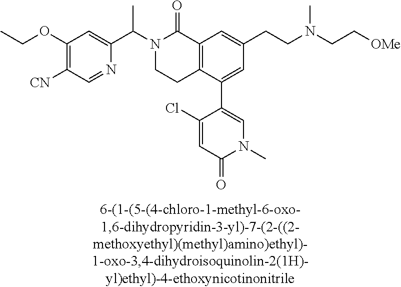 | ¹H NMR (400 MHz, Methanol-d₄) δ ppm 8.62 (d, J = 7.5 Hz, 1H), 7.94 (d, J = 1.6 Hz, 1H), 7.71 (d, J = 14.4 Hz, 1H), 7.31 (s, 1H), 7.18 (d, J = 1.6 Hz, 1H), 6.74 (d, J = 5.1 Hz, 1H), 5.97 (dd, J = 7.1, 2.6 Hz, 1H), 4.31 (dd, J = 7.0, 1.9 Hz, 2H), 3.57 (t, J = 6.4 Hz, 6H), 3.49 (d, J = 6.9 Hz, 1H), 3.37-3.33 (m, 3H), 2.97-2.69 (m, 8H), 2.48 (s, 3H), 1.65 (dd, J = 7.1, 4.1 Hz, 3H), 1.51-1.35 (m, 3H). LC-MS: [M + H]⁺ = 578 | Step 1: 2-(methylamino) ethan-1-ol was replaced with 2- methoxy-N- methylethan-1- amine Step 3: 3-bromo-5-fluoro- 2-(trifluoromethyl) pyridine was replaced with 5-bromo-4-chloro- 1-methylpyridin- 2(1H)-one |
| 234 | 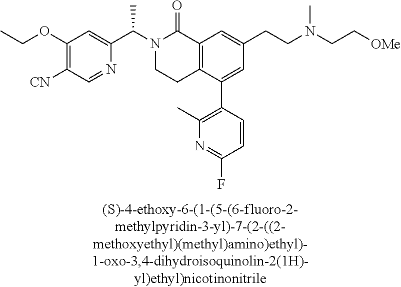 | ¹H NMR (400 MHz, Methanol-d₄) δ ppm 8.61 (d, J = 2.8 Hz, 1H), 7.94 (d, J = 1.8 Hz, 1H), 7.68 (dt, J = 12.7, 8.1 Hz, 1H), 7.28 (d, J = 1.8 Hz, 1H), 7.18 (d, J = 3.4 Hz, 1H), 6.97 (ddd, J = 8.3, 5.8, 2.5 Hz, 1H), 5.98 (qd, J = 7.1, 2.8 Hz, 1H), 4.31 (qd, J = 7.1, 2.8 Hz, 2H), 3.55 (t, J = 5.5 Hz, 3H), 3.46 (dd, J = 7.2, 5.5 Hz, 1H), 3.33 (s, 3H), 2.92 (dd, J = 9.9, 4.8 Hz, 2H), 2.81 (dt, J = 19.8, 4.7 Hz, 4H), 2.65-2.52 (m, 2H), 2.45 (s, 3H), 2.22 (d, J = 17.3 Hz, 3H), 1.65 (dd, J = 7.1, 1.9 Hz, 3H), 1.47 (t, J = 7.0 Hz, 3H). LC-MS: [M + H]⁺ = 546 | Step 1: 2-(methylamino) ethan-1-ol was replaced with 2- methoxy-N- methylethan-1- amine Step 3: 3-bromo-5-fluoro- 2-(trifluoromethyl) pyridine was replaced with 3-bromo-6-fluoro- 2-methylpyridine |
| 235 | 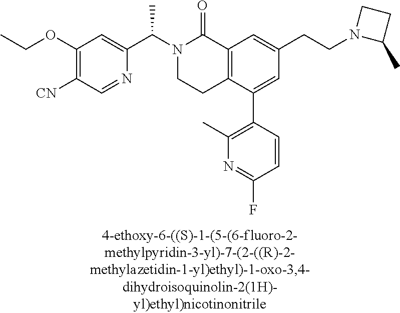 | ¹H NMR (400 MHz, Methanol-d₄) δ ppm 8.60 (d, J = 3.2 Hz, 1H), 7.94 (d, J = 1.9 Hz, 1H), 7.68 (dt, J = 13.5, 8.1 Hz, 1H), 7.26 (d, J = 1.9 Hz, 1H), 7.18 (d, J = 3.6 Hz, 1H), 6.97 (ddd, J = 8.6, 6.1, 2.7 Hz, 1H), 5.98 (qd, J = 7.1, 2.6 Hz, 1H), 4.31 (qd, J = 7.0, 3.0 Hz, 2H), 3.58-3.38 (m, 4H), 2.94 (dd, J = 13.4, 7.8 Hz, 2H), 2.82- 2.51 (m, 5H), 2.28-2.11 (m, 4H), 1.85 (p, J = 9.4 Hz, 1H), 1.64 (dd, J = 7.1, 2.2 Hz, 3H), 1.47 (t, J = 7.0 Hz, 3H), 1.29-1.25 (m, 3H). LC-MS: [M + H]⁺ = 528 | Step 1: 2-(methylamino) ethan-1-ol was replaced with (R)- 2-methylazetidine Step 3: 3-bromo-5-fluoro- 2- (trifluoromethyl) pyridine was replaced with 3-bromo-6-fluoro- 2-methylpyridine |
| 236 | 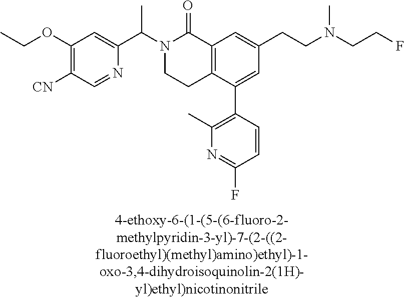 | ¹H NMR (400 MHz, Methanol-d₄) δ ppm 8.61 (d, J = 3.1 Hz, 1H), 8.00 (d, J = 1.7 Hz, 1H), 7.69 (dt, J = 11.8, 8.1 Hz, 1H), 7.36 (d, J = 1.7 Hz, 1H), 7.20 (d, J = 3.2 Hz, 1H), 6.99 (ddd, J = 8.2, 5.5, 2.6 Hz, 1H), 5.96 (dd, J = 7.1, 2.8 Hz, 1H), 4.93 (t, J = 4.5 Hz, 1H), 4.81 (t, J = 4.5 Hz, 1H), 4.38-4.28 (m, 2H), 3.69-3.43 (m, 6H), 3.22- 3.12 (m, 2H), 3.04 (s, 3H), 2.63 (tt, J = 10.1, 5.0 Hz, 2H), 2.22 (d, J = 16.2 Hz, 3H), 1.65 (dd, J = 7.1, 1.7 Hz, 3H), 1.48 (t, J = 7.0 Hz, 3H). LC-MS: [M + H]⁺ = 534 | Step 1: 2-(methylamino) ethan-1-ol was replaced with 2- fluoro-N- methylethan-1- amine Step 3: 3-bromo-5-fluoro- 2- (trifluoromethyl) pyridine was replaced with 3-bromo-6-fluoro- 2-methylpyridine |
| 237 | 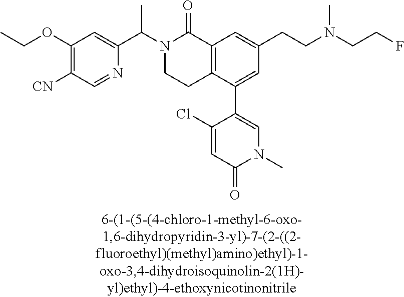 | ¹H NMR (400 MHz, Methanol-d₄) δ 8.62 (d, J = 7.7 Hz, 1H), 7.93 (d, J = 1.7 Hz, 1H), 7.70 (d, J = 14.4 Hz, 1H), 7.31 (s, 1H), 7.18 (d, J = 2.1 Hz, 1H), 6.74 (d, J = 5.2 Hz, 1H), 5.97 (dt, J = 7.0, 3.5 Hz, 1H), 4.63 (t, J = 4.5 Hz, 1H), 4.52 (t, J = 4.6 Hz, 1H), 4.31 (qd, J = 7.0, 2.1 Hz, 2H), 3.57 (d, J = 6.0 Hz, 4H), 3.52-3.43 (m, 1H), 2.96-2.87 (m, 3H), 2.87-2.71 (m, 5H), 2.44 (s, 3H), 1.65 (dd, J = 7.1, 4.3 Hz, 3H), 1.56-1.37 (m, 3H). LC-MS: [M + H]⁺ = 566 | Step 1: 2-(methylamino) ethan-1-ol was replaced with 2- fluoro-N- methylethan-1- amine Step 3: 3-bromo-5-fluoro- 2-(trifluoromethyl) pyridine was replaced with 5-bromo-4-chloro- 1-methylpyridin- 2(1H)-one |
| 238 | 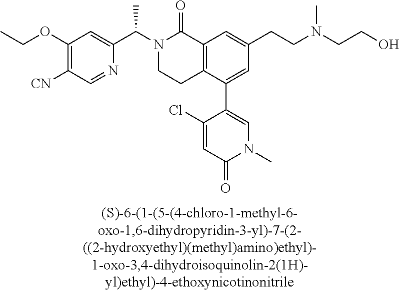 | ¹H NMR (400 MHz, Methanol-d₄) δ 8.62 (d, J = 7.6 Hz, 1H), 7.95 (d, J = 1.8 Hz, 1H), 7.71 (d, J = 14.2 Hz, 1H), 7.33 (s, 1H), 7.19 (d, J = 1.6 Hz, 1H), 6.74 (d, J = 5.1 Hz, 1H), 4.31 (dd, J = 7.0, 1.9 Hz, 2H), 3.73 (t, J = 5.8 Hz, 2H), 3.57 (d, J = 6.0 Hz, 4H), 3.53- 3.42 (m, 1H), 2.96 (s, 4H), 2.84 (t, J = 5.6 Hz, 2H), 2.76 (d, J = 6.0 Hz, 2H), 2.56 (s, 3H), 1.65 (dd, J = 7.1, 4.1 Hz, 3H), 1.47 (t, J = 7.0 Hz, 3H). LC-MS: [M + H]⁺ = 564. | Step 1: 2-(methylamino) ethan-1-ol was replaced with 2- (methylamino) ethan-1-ol Step 3: 3-bromo-5-fluoro- 2-(trifluoromethyl) pyridine was replaced with 5-bromo-4-chloro- 1-methylpyridin- 2(1H)-one |
| 239 | 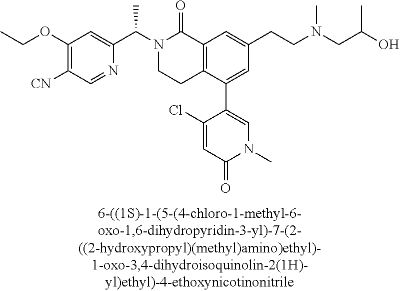 | ¹H NMR (400 MHz, Methanol-d₄) δ 8.61 (d, J = 7.5 Hz, 1H), 7.96 (d, J = 1.7 Hz, 1H), 7.71 (d, J = 14.2 Hz, 1H), 7.34 (s, 1H), 7.19 (d, J = 1.5 Hz, 1H), 6.74 (d, J = 5.2 Hz, 1H), 5.97 (dd, J = 7.1, 2.6 Hz, 1H), 4.35-4.26 (m, 2H), 4.04- 3.93 (m, 1H), 3.58 (d, J = 6.0 Hz, 4H), 3.52-3.44 (m, 1H), 3.12- 2.98 (m, 4H), 2.75 (t, J = 6.8 Hz, 4H), 2.64 (s, 3H), 1.65 (dd, J = 7.1, 4.0 Hz, 3H), 1.47 (t, J = 7.0 Hz, 3H), 1.17 (d, J = 6.2 Hz, 3H). LC-MS: [M + H]⁺ = 578. | Step 1: 2-(methylamino) ethan-1-ol was replaced with 1- (methylamino) propan-2-ol Step 3: 3-bromo-5-fluoro- 2-(trifluoromethyl) pyridine was replaced with 5-bromo-4- chloro-1- methylpyridin- 2(1H)-one |
| 240 | 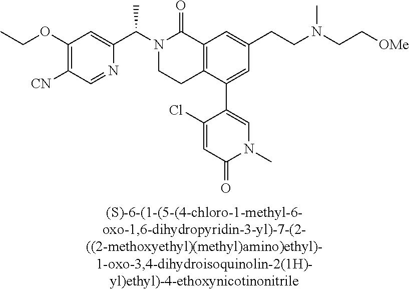 | ¹H NMR (400 MHz, Methanol-d₄) δ 8.62 (d, J = 7.6 Hz, 1H), 7.93 (d, J = 1.7 Hz, 1H), 7.71 (d, J = 14.6 Hz, 1H), 7.30 (s, 1H), 7.18 (d, J = 1.9 Hz, 1H), 6.74 (d, J = 5.2 Hz, 1H), 5.98 (dd, J = 7.1, 2.6 Hz, 1H), 4.38-4.24 (m, 2H), 3.57 (d, J = 6.1 Hz, 4H), 3.53 (t, J = 5.5 Hz, 3H), 3.33 (s, 3H), 2.88 (dd, J = 10.1, 5.9 Hz, 2H), 2.79-2.65 (m, 6H), 2.38 (s, 3H), 1.65 (dd, J = 7.1, 4.2 Hz, 3H), 1.51-1.44 (m, 3H). LC-MS: [M + H]⁺ = 578. | Step 1: 2-(methylamino) ethan-1-ol was replaced with 2- methoxy-N- methylethan-1- amine Step 3: 3-bromo-5-fluoro- 2-(trifluoromethyl) pyridine was replaced with 5-bromo-4- chloro-1- methylpyridin- 2(1H)-one |
| 241 | 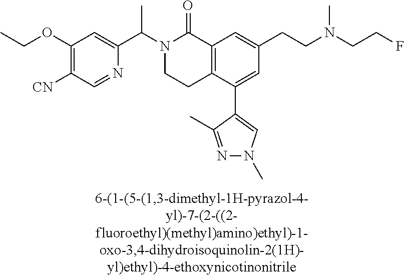 | ¹H NMR (400 MHz, Methanol-d₄) δ 8.65 (s, 1H), 8.51 (s, 1H), 7.88 (d, J = 1.9 Hz, 1H), 7.55 (d, J = 5.0 Hz, 1H), 7.33 (d, J = 1.9 Hz, 1H), 7.21 (s, 1H), 6.01 (q, J = 7.1 Hz, 1H), 4.72-4.67 (m, 1H), 4.62- 4.54 (m, 1H), 4.34 (q, J = 7.0 Hz, 2H), 3.88 (s, 3H), 3.63-3.42 (m, 2H), 3.02 (t, J = 4.8 Hz, 1H), 2.98-2.78 (m, 7H), 2.54 (s, 3H), 2.15 (s, 3H), 1.68 (d, J = 7.1 Hz, 3H), 1.50 (t, J = 7.0 Hz, 3H). LC-MS: [M + H]⁺ = 519. | Step 1: 2-(methylamino) ethan-1-ol was replaced with 2- fluoro-N- methylethan-1- amine Step 3: 3-bromo-5- fluoro-2- (trifluoromethyl) pyridine was replaced with (1,3-dimethyl- 1H-pyrazol-4- yl)boronic acid |
| 242 | 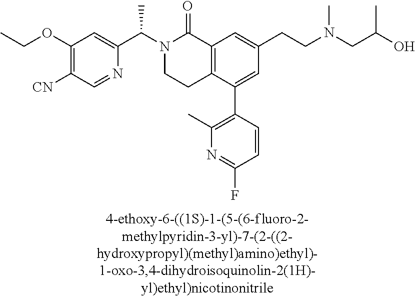 | ¹H NMR (400 MHz, Methanol-d₄) δ 8.61 (d, J = 2.9 Hz, 1H), 7.96 (d, J = 1.7 Hz, 1H), 7.68 (td, J = 8.1, 12.5 Hz, 1H), 7.29 (d, J = 1.8 Hz, 1H), 7.19 (d, J = 3.3 Hz, 1H), 7.01-6.93 (m, 1H), 5.98 (br dd, J = 3.0, 7.2 Hz, 1H), 4.37-4.26 (m, 2H), 3.90 (br s, 1H), 3.62-3.41 (m, 2H), 2.96 (br s, 4H), 2.70-2.45 (m, 7H), 2.22 (d, J = 17.1 Hz, 3H), 1.65 (dd, J = 1.9, 7.2 Hz, 3H), 1.50-1.45 (m, 4H), 1.13 (d, J = 6.1 Hz, 3H). LC-MS: [M + H]⁺ = 546. | Step 1: 2-(methylamino) ethan-1-ol was replaced with 1- (methylamino) propan-2-ol Step 3: 3-bromo-5- fluoro-2- (trifluoromethyl) pyridine was replaced with 3-bromo-6- fluoro-2- methylpyridine |
| 243 | 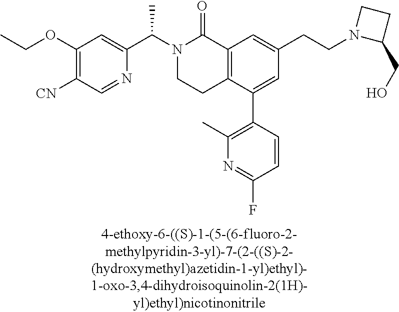 | ¹H NMR (400 MHz, Methanol-d₄) δ 8.61 (d, J = 3.0 Hz, 1H), 7.93 (d, J = 1.9 Hz, 1H), 7.68 (dt, J = 11.7, 8.1 Hz, 1H), 7.29-7.16 (m, 2H), 6.97 (ddd, J = 8.3, 5.7, 2.7 Hz, 1H), 5.98 (qd, J = 7.1, 2.8 Hz, 1H), 4.58 (s, 1H), 4.38-4.25 (m, 2H), 3.68-3.34 (m, 5H), 3.04- 2.88 (m, 2H), 2.86-2.48 (m, 5H), 2.22 (d, J = 17.7 Hz, 3H), 2.10- 1.90 (m, 2H), 1.64 (dd, J = 7.1, 2.1 Hz, 3H), 1.52-1.43 (m, 3H). LC-MS: [M + H]⁺ = 544 | Step 1: 2-(methylamino) ethan-1-ol was replaced with (S)-azetidin-2- ylmethanol Step 3: 3-bromo-5- fluoro-2- (trifluoromethyl) pyridine was replaced with 3-bromo-6- fluoro-2- methylpyridine |
| 244 | 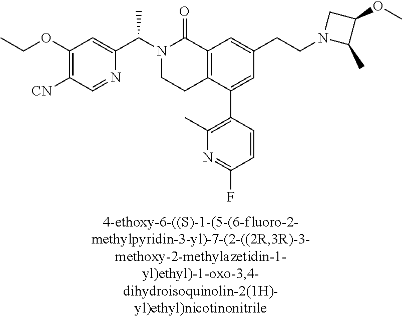 | ¹H NMR (400 MHz, Methanol-d₄) δ 8.61 (d, J = 3.1 Hz, 1H), 7.93 (d, J = 1.9 Hz, 1H), 7.67 (dt, J = 13.7, 8.1 Hz, 1H), 7.25 (d, J = 1.9 Hz, 1H), 7.18 (d, J = 3.7 Hz, 1H), 6.97 (ddd, J = 8.6, 6.0, 2.7 Hz, 1H), 5.98 (qd, J = 7.1, 2.8 Hz, 1H), 4.31 (pd, J = 6.8, 3.4 Hz, 2H), 4.01-3.91 (m, 1H), 3.59- 3.40 (m, 3H), 3.37-3.32 (m, 1H), 3.29 (d, J = 1.5 Hz, 3H), 3.05 (ddd, J = 9.1, 5.8, 1.3 Hz, 1H), 2.93-2.68 (m, 3H), 2.67-2.49 (m, 3H), 2.21 (d, J = 18.1 Hz, 3H), 1.64 (dd, J = 7.1, 2.2 Hz, 3H), 1.51-1.45 (m, 3H), 1.20 (d, J = 6.6 Hz, 3H). LC-MS: [M + H]⁺ = 558. | Step 1: 2-(methylamino) ethan-1-ol was replaced with 2R,3R)-3- methoxy-2- methylazetidine Step 3: 3-bromo-5- fluoro-2- (trifluoromethyl) pyridine was replaced with 3-bromo-6- fluoro-2- methylpyridine |
| 245 | 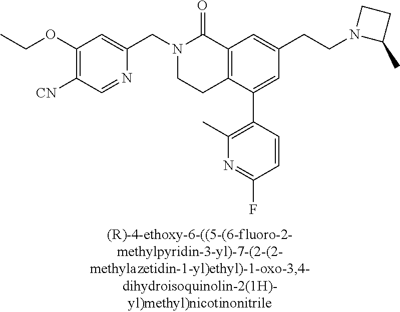 | ¹H NMR (400 MHz, Methanol-d₄) δ 8.60 (s, 1H), 7.94 (d, J = 1.8 Hz, 1H), 7.72 (t, J = 8.0 Hz, 1H), 7.27 (d, J = 1.8 Hz, 1H), 7.18 (s, 1H), 6.99 (dd, J = 2.4, 8.4 Hz, 1H), 4.88 (d, J = 2.4 Hz, 2H), 4.33 (q, J = 7.0 Hz, 2H), 3.70-3.60 (m, 2H), 3.38- 3.34 (m, 1H), 2.94-2.55 (m, 8H), 2.24 (s, 3H), 2.11 (dtd, J = 2.0, 7.8, 10.2 Hz, 1H), 1.80 (quin, J = 9.4 Hz, 1H), 1.47 (t, J = 7.0 Hz, 3H), 1.23 (d, J = 6.0 Hz, 3H). LC-MS: [M + H]⁺ = 514. | Step 1: 2-(methylamino) ethan-1-ol was replaced with R)- 2-methylazetidine Step 3: 3-bromo-5- fluoro-2- (trifluoromethyl) pyridine was replaced with 3-bromo-6- fluoro-2- methylpyridine |
| 246 | 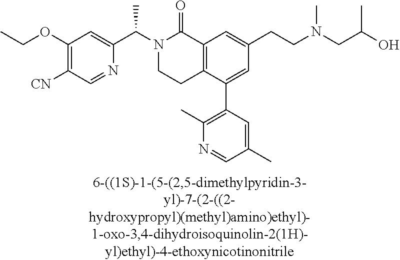 | ¹H NMR (400 MHz, Methanol-d₄) δ 8.63 (d, J = 2.7 Hz, 1H), 8.55 (br s, 1H), 8.33 (s, 1H), 7.99 (s, 1H), 7.45 (br d, J = 12.1 Hz, 1H), 7.32 (s, 1H), 7.21 (d, J = 2.8 Hz, 1H), 5.99 (br dd, J = 3.2, 7.1 Hz, 1H), 4.39-4.29 (m, 2H), 4.09- 3.98 (m, 1H), 3.64-3.41 (m, 2H), 3.25-2.98 (m, 4H), 2.94-2.80 (m, 2H), 2.74 (s, 3H), 2.70- 2.51 (m, 2H), 2.38 (d, J = 5.4 Hz, 3H), 2.25 (d, J = 16.9 Hz, 3H), 1.72-1.63 (m, 3H), 1.50 (t, J = 7.0 Hz, 3H), 1.20 (d, J = 6.1 Hz, 3H) LC-MS: [M + H]⁺ = 542. | Step 1: 2-(methylamino) ethan-1-ol was replaced with 1- (methylamino) propan-2-ol Step 3: 3-bromo-5- fluoro-2- (trifluoromethyl) pyridine was replaced with 3-bromo-2,5- dimethylpyridine |
| 247 | 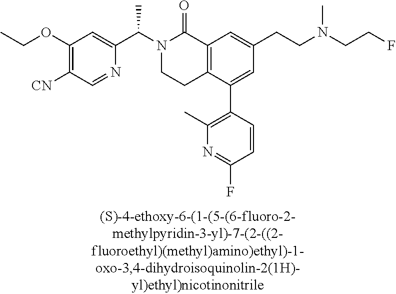 | ¹H NMR (400 MHz, Methanol-d₄) δ 8.61 (d, J = 3.1 Hz, 1H), 8.00 (d, J = 1.7 Hz, 1H), 7.69 (dt, J = 11.8, 8.1 Hz, 1H), 7.36 (d, J = 1.7 Hz, 1H), 7.20 (d, J = 3.2 Hz, 1H), 6.99 (ddd, J = 8.2, 5.5, 2.6 Hz, 1H), 5.96 (dd, J = 7.1, 2.8 Hz, 1H), 4.93 (t, J = 4.5 Hz, 1H), 4.81 (t, J = 4.5 Hz, 1H), 4.38-4.28 (m, 2H), 3.69-3.43 (m, 6H), 3.22- 3.12 (m, 2H), 3.04 (s, 3H), 2.63 (tt, J = 10.1, 5.0 Hz, 2H), 2.22 (d, J = 16.2 Hz, 3H), 1.65 (dd, J = 7.1, 1.7 Hz, 3H), 1.48 (t, J = 7.0 Hz, 3H). LC-MS: [M + H]⁺ = 534. | Step 1: 2-(methylamino) ethan-1-ol was replaced with 2- fluoro-N- methylethan-1- amine Step 3: 3-bromo-5- fluoro-2- (trifluoromethyl) pyridine was replaced with 3-bromo-6- fluoro-2- methylpyridine |
| 248 | 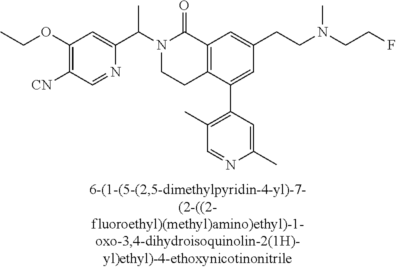 | ¹H NMR (400 MHz, Methanol-d₄) δ 8.61 (s, 1H), 8.34 (d, J = 4.2 Hz, 2H), 7.97 (d, J = 1.7 Hz, 1H), 7.27 (d, J = 1.8 Hz, 1H), 7.19 (d, J = 3.2 Hz, 1H), 7.07 (d, J = 11.5 Hz, 1H), 5.97 (qd, J = 7.1, 2.7 Hz, 1H), 4.78-4.71 (m, 1H), 4.67- 4.57 (m, 1H), 4.37-4.20 (m, 2H), 3.55 (dt, J = 8.3, 4.6 Hz, 1H), 3.50-3.39 (m, 1H), 3.26-3.06 (m, 4H), 3.03 (dd, J = 10.0, 4.6 Hz, 2H), 2.70 (s, 3H), 2.63 (dd, J = 8.4, 3.9 Hz, 1H), 2.58-2.45 (m, 4H), 2.04 (d, J = 16.6 Hz, 3H), 1.65 (d, J = 7.1 Hz, 3H), 1.47 (t, J = 7.0 Hz, 3H). LC-MS: [M + H]⁺ = 530. | Step 1: 2-(methylamino) ethan-1-ol was replaced with 2- fluoro-N- methylethan-1- amine Step 3: 3-bromo-5- fluoro-2- (trifluoromethyl) pyridine was replaced with 4-bromo-2,5- dimethylpyridine |
| 249 | 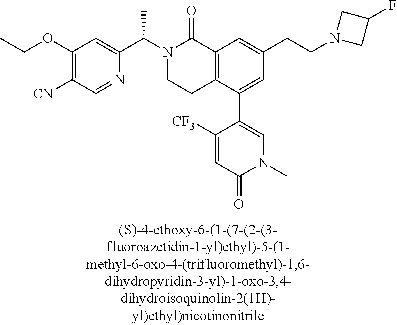 | ¹H NMR (400 MHz, Methanol-d₄) δ 8.61 (d, J = 1.2 Hz, 1H), 7.93 (d, J = 1.9 Hz, 1H), 7.68 (d, J = 17.0 Hz, 1H), 7.35-7.24 (m, 1H), 7.18 (d, J = 5.9 Hz, 1H), 6.94 (d, J = 3.5 Hz, 1H), 5.97 (t, J = 7.1 Hz, 1H), 5.23-5.00 (m, 1H), 4.30 (qdd, J = 7.0, 3.1, 1.5 Hz, 2H), 3.73-3.54 (m, 6H), 3.54-3.46 (m, 1H), 3.33 (s, 1H), 3.26-3.20 (m, 1H), 2.89-2.70 (m, 5H), 2.60 (s, 1H), 1.64 (dd, J = 7.1, 3.3 Hz, 3H), 1.47 (td, J = 7.0, 4.3 Hz, 3H). LC-MS: [M + H]⁺ = 598. | Step 1: 2-(methylamino) ethan-1-ol was replaced with 3- fluoroazetidine Step 3: 3-bromo-5- fluoro-2- (trifluoromethyl) pyridine was replaced with 5-bromo-1- methyl-4- (trifluoromethyl) pyridin-2(1H)- one |
| 250 | 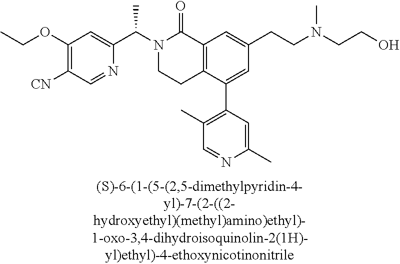 | ¹H NMR (400 MHz, Methanol-d₄) δ 8.61 (s, 1H), 8.33 (d, J = 4.3 Hz, 1H), 7.94 (d, J = 1.6 Hz, 1H), 7.20 (dd, J = 16.1, 2.6 Hz, 2H), 7.07 (d, J = 12.1 Hz, 1H), 5.98 (dd, J = 7.1, 2.7 Hz, 1H), 4.31 (qd, J = 7.0, 1.5 Hz, 2H), 3.66 (t, J = 6.0 Hz, 2H), 3.53 (s, 1H), 3.47 (s, 1H), 2.89 (d, J = 9.0 Hz, 2H), 2.76 (d, J = 9.0 Hz, 2H), 2.64 (t, J = 6.0 Hz, 3H), 2.51 (d, J = 5.4 Hz, 4H), 2.39 (s, 3H), 2.04 (d, J = 17.1 Hz, 3H), 1.64 (d, J = 7.1 Hz, 3H), 1.47 (t, J = 7.0 Hz, 3H). LC-MS: [M + H]⁺ = 528. | Step 1: 2-(methylamino) ethan-1-ol was replaced with 2- (methylamino) ethan-1-ol Step 3: 3-bromo-5- fluoro-2- (trifluoromethyl) pyridine was replaced with 4-bromo-2,5- dimethylpyridine |
| 251 | 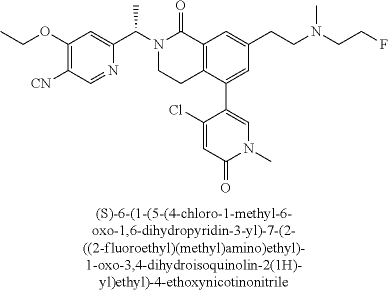 | ¹H NMR (400 MHz, Methanol-d₄) δ 8.62 (d, J = 7.7 Hz, 1H), 7.93 (d, J = 1.7 Hz, 1H), 7.70 (d, J = 14.4 Hz, 1H), 7.31 (s, 1H), 7.18 (d, J = 2.1 Hz, 1H), 6.74 (d, J = 5.2 Hz, 1H), 5.97 (dt, J = 7.0, 3.5 Hz, 1H), 4.63 (t, J = 4.5 Hz, 1H), 4.52 (t, J = 4.6 Hz, 1H), 4.31 (qd, J = 7.0, 2.1 Hz, 2H), 3.57 (d, J = 6.0 Hz, 4H), 3.52-3.43 (m, 1H), 2.96-2.87 (m, 3H), 2.87-2.71 (m, 5H), 2.44 (s, 3H), 1.65 (dd, J = 7.1, 4.3 Hz, 3H), 1.56-1.37 (m, 3H). LC-MS: [M + H]⁺ = 566. | Step 1: 2-(methylamino) ethan-1-ol was replaced with 2- fluoro-N- methylethan-1- amine Step 3: 3-bromo-5- fluoro-2- (trifluoromethyl) pyridine was replaced with 5-bromo-4- chloro-1- methylpyridin- 2(1H)-one |
| 252 | 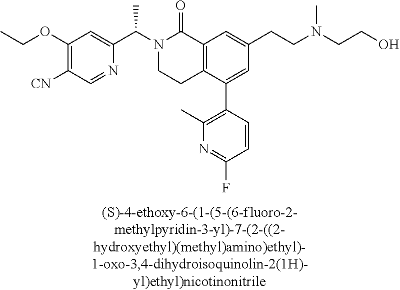 | ¹H NMR (400 MHz, Methanol-d₄) δ 8.61 (d, J = 2.9 Hz, 1H), 7.95 (d, J = 1.8 Hz, 1H), 7.68 (dt, J = 12.5, 8.1 Hz, 1H), 7.28 (d, J = 1.8 Hz, 1H), 7.18 (d, J = 3.4 Hz, 1H), 6.97 (ddd, J = 8.3, 5.7, 2.5 Hz, 1H), 5.98 (dd, J = 7.1, 2.9 Hz, 1H), 4.31 (qd, J = 7.1, 3.0 Hz, 2H), 3.69 (t, J = 5.9 Hz, 2H), 3.55 (ddd, J = 13.7, 8.4, 5.3 Hz, 1H), 3.51-3.40 (m, 1H), 2.93 (s, 4H), 2.76 (s, 2H), 2.60 (s, 1H), 2.49 (s, 2H), 2.22 (d, J = 17.1 Hz, 3H), 1.65 (dd, J = 7.1, 1.9 Hz, 3H), 1.47 (t, J = 7.0 Hz, 3H). LC-MS: [M + H]⁺ = 532. | Step 1: 2-(methylamino) ethan-1-ol was replaced with 2- (methylamino) ethan-1-ol Step 3: 3-bromo-5- fluoro-2- (trifluoromethyl) pyridine was replaced with 3-bromo-6- fluoro-2- methylpyridine |
| 253 | 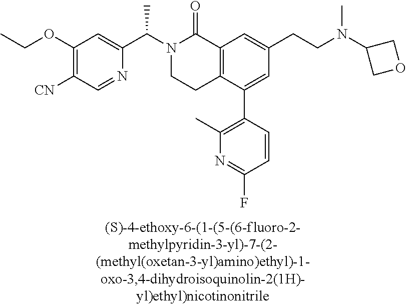 | ¹H NMR (400 MHz, Methanol-d₄) δ 8.61 (d, J = 2.7 Hz, 1H), 7.93 (d, J = 1.8 Hz, 1H), 7.68 (dt, J = 12.6, 8.1 Hz, 1H), 7.27 (d, J = 1.9 Hz, 1H), 7.18 (d, J = 3.4 Hz, 1H), 6.97 (ddd, J = 8.4, 5.9, 2.6 Hz, 1H), 5.98 (qd, J = 7.1, 3.0 Hz, 1H), 4.64 (t, J = 6.7 Hz, 2H), 4.53 (t, J = 6.3 Hz, 2H), 4.35-4.21 (m, 2H), 3.65 (p, J = 6.5 Hz, 1H), 3.58-3.47 (m, 1H), 3.47-3.36 (m, 1H), 2.90-2.78 (m, 2H), 2.63- 2.47 (m, 4H), 2.28-2.15 (m, 6H), 1.64 (dd, J = 7.1, 1.8 Hz, 3H), 1.47 (t, J = 7.0 Hz, 3H). LC-MS: [M + H]⁺ = 544. | Step 1: 2-(methylamino) ethan-1-ol was replaced with N- methyloxetan-3- amine Step 3: 3-bromo-5- fluoro-2- (trifluoromethyl) pyridine was replaced with 3-bromo-6- fluoro-2- methylpyridine |
| 254 | 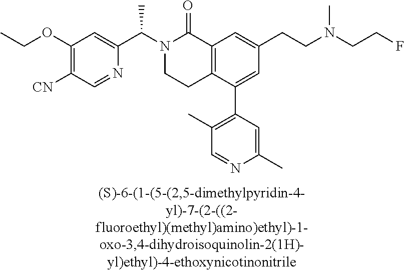 | ¹H NMR (400 MHz, Methanol-d₄) δ 8.61 (s, 1H), 8.34 (d, J = 4.2 Hz, 2H), 7.97 (d, J = 1.7 Hz, 1H), 7.27 (d, J = 1.8 Hz, 1H), 7.19 (d, J = 3.2 Hz, 1H), 7.07 (d, J = 11.5 Hz, 1H), 5.97 (qd, J = 7.1, 2.7 Hz, 1H), 4.78-4.71 (m, 1H), 4.67- 4.57 (m, 1H), 4.37-4.20 (m, 2H), 3.55 (dt, J = 8.3, 4.6 Hz, 1H), 3.50-3.39 (m, 1H), 3.26-3.06 (m, 4H), 3.03 (dd, J = 10.0, 4.6 Hz, 2H), 2.70 (s, 3H), 2.63 (dd, J = 8.4, 3.9 Hz, 1H), 2.58-2.45 (m, 4H), 2.04 (d, J = 16.6 Hz, 3H), 1.65 (d, J = 7.1 Hz, 3H), 1.47 (t, J = 7.0 Hz, 3H). LC-MS: [M + H]⁺ = 530. | Step 1: 2-(methylamino) ethan-1-ol was replaced with 2-fluoro-N- methylethan-1- amine Step 3: 3-bromo-5- fluoro-2- (trifluoromethyl) pyridine was replaced with 4-bromo-2,5- dimethylpyridine |
| 255 | 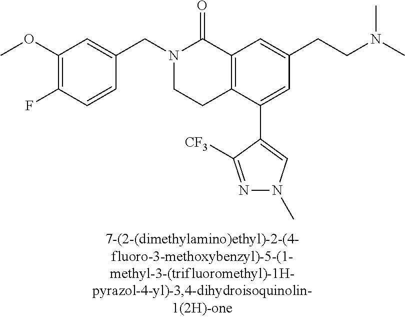 | ¹H NMR (400 MHz, Methanol-d₄) δ 8.01 (s, 1H), 7.75 (s, 1H), 7.40 (s, 1H), 7.07 (s, 2H), 6.89 (s, 1H), 4.74 (s, 2H), 3.99 (s, 3H), 3.84 (s, 3H), 3.44 (d, J = 22.4 Hz, 4H), 3.13 (s, 2H), 2.96 (s, 6H), 2.76 (s, 2H). LC-MS: [M + H]⁺ = 505. | Step 1: 2-(methylamino) ethan-1-ol was replaced with dimethylamine Step 3: 3-bromo-5- fluoro-2- (trifluoromethyl) pyridine was replaced with 4-bromo-1- methyl-3- (trifluoromethyl)- 1H-pyrazole |

### Example 256: Synthesis of 4-ethoxy-6-((1S)-1-(7-(2-(ethyl(methyl)amino)ethyl)-5-(1-hydroxy-2-methylpropyl)-1-oxo-3,4-dihydroisoquinolin-2(1H)-yl)ethyl)nicotinonitrile (256)

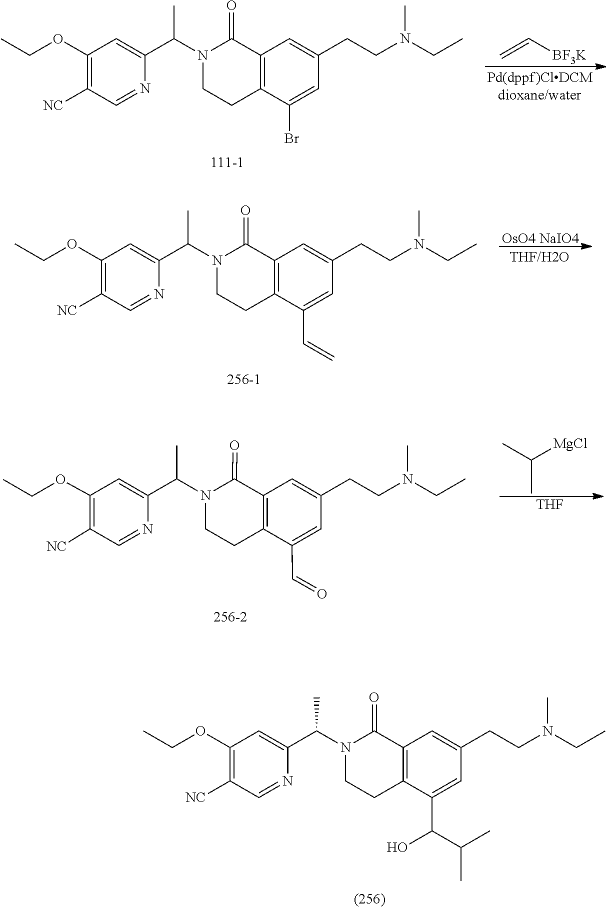

Step 1: To a mixture of compound 111-1 (1.32 g, 2.72 mmol, 1 equiv) and compound trifluoro(vinyl)-lambda⁴-borane, potassium salt (1.82 g, 13.6 mmol, 5.0 equiv) in dioxane (24 mL) was added K₂CO₃ (1.128 g, 8.2 mmol, 3.0 equiv) in water (24 mL) and Pd(dppf)Cl2.DCM (222 mg, 0.28 mmol, 0.1 eq) at 25° C. The reaction mixture was stirred at 100° C. for 16 hrs under N₂. TLC (dichloromethane/methanol 10:1) showed trace compound 111-1 remained. The mixture was combined with another batch (from compound 900 mg of 111-1). The mixture was diluted with water (300 mL). The resulting solution was extracted with ethyl acetate (300 mL×2). The organic layer was dried over Na₂SO₄, filtered and concentrated. The residue was purified by column chromatography eluting with ethyl acetate/methanol (gradient: 0˜50% of ethyl acetate, 5% Et₃N) to afford 4-ethoxy-6-(1-(7-(2-(ethyl(methyl)amino)ethyl)-1-oxo-5-vinyl-3,4-dihydroisoquinolin-2(1H)-yl)ethyl)nicotinonitrile (256-1) (1.9 g, 61.8% yield) as brown oil. LCMS: 433.1 [M+1]+

Step 2: To a mixture of compound 256-1 (1.67 g, 3.86 mmol, 1 equiv) in THF/H₂O (36 mL/12 mL) was added OsO₄ (147 mg, 0.578 mmol, 0.15 eq) at 25° C. The reaction mixture was stirred at 25° C. for 0.5 hrs. NaIO₄ (4.13 g, 0.019 mmol, 4.9 eq) was added. The reaction mixture was stirred at 25° C. for 16 hrs. The mixture was diluted with water (500 mL). The mixture was extracted with ethyl acetate (500 mL×3). The organic layer was dried over Na₂SO₄, filtered and concentrated. The residue was purified by column chromatography eluting with ethyl acetate/methanol (gradient: 0-50% of methanol, 5% NEt₃) to afford the 4-ethoxy-6-(1-(7-(2-(ethyl(methyl)amino)ethyl)-5-formyl-1-oxo-3,4-dihydroisoquinolin-2(1H)-yl)ethyl)nicotinonitrile (256-2) (810 mg, 42% yield) as brown gum. LCMS: 435.1 [M+1]+.

Step 3: To a mixture of compound 256-2 (350 mg, 0.8 mmol, 1 equiv) in THF (7 mL) was added isopropylmagnesium chloride (1 mL, 2 M in THF 2 mmol, 2.5 equiv) at 0° C. The mixture was stirred at 0° C. for 20 min. The mixture was diluted with sat. NH₄Cl (100 mL) and water (100 mL). The resulting solution was extracted with ethyl acetate (200 mL×3). The organic layer was dried over Na₂SO₄, filtered and concentrated. The residue was purified prep-HPLC (column: phenomenex luna C18 200*40*10 um; Gradient: 16-46% of B, A=0.1% TFA in water, B=acetonitrile, flow rate: 60 mL/min) to afford racemate product (55 mg) as a yellow solid, which was purified by Prep-SFC (column: DAICEL CHIRALPAK IC (250 mm*30 mm, 10 um); Gradient Time: 3.8 min; 95 min; Condition: 0.1% NH₃H₂O MeOH; Flow Rate: 70 g/min; 70% of B) to give 4-ethoxy-6-((1S)-1-(7-(2-(ethyl(methyl)amino)ethyl)-5-(1-hydroxy-2-methylpropyl)-1-oxo-3,4-dihydroisoquinolin-2(1H)-yl)ethyl)nicotinonitrile (256) as a yellow solid. ¹H NMR (400 MHz, Methanol-d₄) δ ppm 8.66-8.60 (m, 1H), 7.84 (s, 1H), 7.56 (br s, 1H), 7.19 (s, 1H), 6.01-5.91 (m, 1H), 4.62-4.57 (m, 1H), 4.31 (q, J=6.9 Hz, 2H), 3.67-3.53 (m, 2H), 3.53-3.45 (m, 1H), 3.44-3.36 (m, 2H), 3.17-2.97 (m, 4H), 2.96-2.85 (m, 4H), 1.98-1.84 (m, 1H), 1.72-1.63 (m, 3H), 1.53-1.44 (m, 3H), 1.36 (t, J=7.3 Hz, 4H), 1.05-0.94 (m, 3H), 0.85-0.76 (m, 3H). LC-MS: [M+H]⁺=479.

Following a similar procedure to that of Example 256 (unless otherwise specified). The following compounds were prepared from their corresponding precursors

| Ex. | Name/Structure | ¹HNMR/LC-MS | Precursors |
|---|---|---|---|
| 257 | 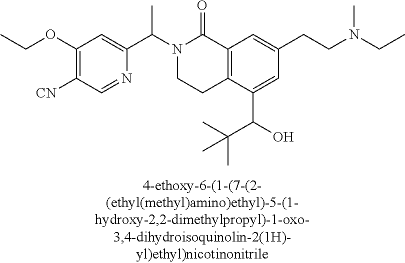 | ¹H NMR (400 MHz, Methanol-d₄) δ 8.61 (s, 1H), 7.79 (d, J = 2.0 Hz, 1H), 7.54 (d, J = 2.0 Hz, 1H), 7.17 (s, 1H), 5.96 (d, J = 7.1 Hz, 1H), 4.70 (s, 1H), 4.30 (qd, J = 7.0, 1.5 Hz, 2H), 3.67-3.55 (m, 1H), 3.43- 3.34 (m, 1H), 2.99 (d, J = 5.9 Hz, 2H), 2.86 (dd, J = 10.6, 5.6 Hz, 2H), 2.69 (dd, J = 8.6, 3.3 Hz, 2H), 2.58 (q, J = 7.2 Hz, 2H), 2.35 (s, 3H), 1.67 (d, J = 7.2 Hz, 3H), 1.47 (t, J = 7.0 Hz, 3H), 1.11 (t, J = 7.2 Hz, 3H), 0.92 (s, 9H). LC- MS: [M + H]⁺ = 493. | Step 3: isopropylmagnesium chloride was replaced with tert- butylmagnesium chloride |

### Example 258: 4-ethoxy-6-(1-(7-(2-(ethyl(methyl)amino)ethyl)-1-oxo-5-(3,3,3-trifluoro-1-hydroxy-2-methylpropyl)-3,4-dihydroisoquinolin-2(1H)-yl)ethyl)nicotinonitrile (258)

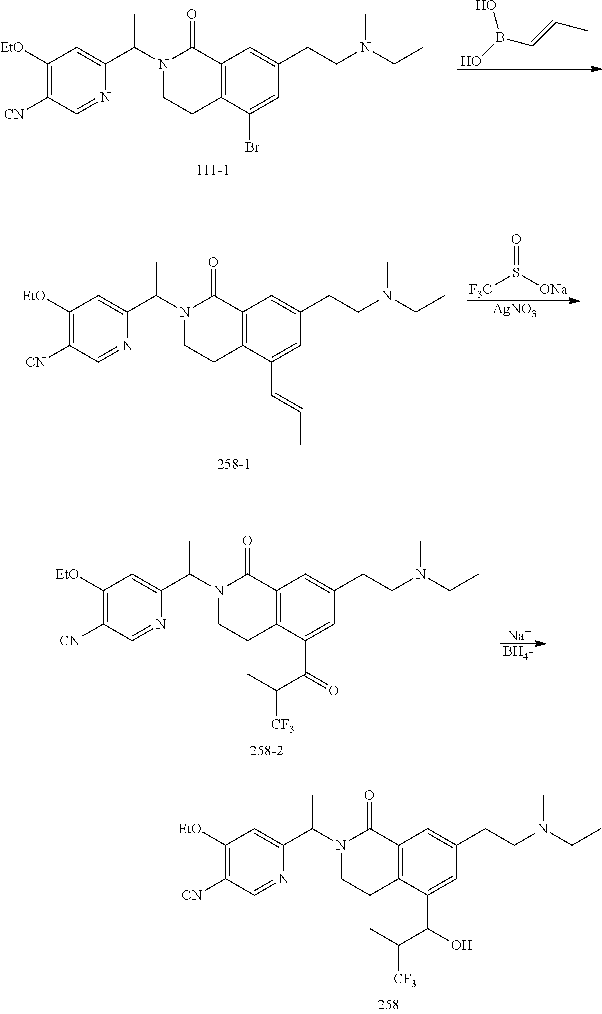

Step 1: To a reaction tube was added compound 111-1 (200 mg, 0.412 mmol), (E)-prop-1-en-1-ylboronic acid (70.8 mg, 0.824 mmol), PdCl₂(dppf).CH₂Cl₂ adduct (33.6 mg, 0.041 mmol) and Na₂CO₃ (131 mg, 1.236 mmol) in N₂, 1,4-dioxane (3 mL) and water (0.300 mL) was added and stirred at 100° C. for 20 hrs. The solvent was evaporated and the residue purified by flash column (20% MeOH in CH₂Cl₂) to give (E)-4-ethoxy-6-(1-(7-(2-(ethyl(methyl)amino)ethyl)-1-oxo-5-(prop-1-en-1-yl)-3,4-dihydroisoquinolin-2(1H)-yl)ethyl)nicotinonitrile (258-1) (100 mg, 55% yield). LCMS: 447.3 [M+H]⁺.

Step 2: To a solution of compound 258-1 (100 mg, 0.224 mmol) in DMF (1 mL) was added sodium trifluoromethanesulfinate (69.9 mg, 0.448 mmol), (nitrooxy)silver (11.41 mg, 0.067 mmol) and potassium persulfate (18.16 mg, 0.067 mmol) and stirred at RT for 24 hrs. The mixture was filtered, the filtrate was diluted with EtOAc and washed with brine, dried and concentrated. The residue was purified by flash column (30% MeOH in CH₂Cl₂) to give 4-ethoxy-6-(1-(7-(2-(ethyl(methyl)amino)ethyl)-1-oxo-5-(3,3,3-trifluoro-2-methylpropanoyl)-3,4-dihydroisoquinolin-2(1H)-yl)ethyl)nicotinonitrile (258-2). LCMS 531.3 [M+H]⁺.

Step 3: To the solution of 258-2 (30 mg, 0.057 mmol) in MeOH (1 mL) was added NaBH₄ (4.28 mg, 0.113 mmol) and stirred at RT for 2 hrs. The reaction was quenched with acetone and the mixture purified by HPLC (column: phenomenex luna C18 200*40*10 um, Gradient: 16-46% of B, A=0.1% TFA in water, B=acetonitrile, flow rate: 60 mL/min) to afford 4-ethoxy-6-(1-(7-(2-(ethyl(methyl)amino)ethyl)-1-oxo-5-(3,3,3-trifluoro-1-hydroxy-2-methylpropyl)-3,4-dihydroisoquinolin-2(1H)-yl)ethyl)nicotinonitrile (258). ¹H NMR (400 MHz, Methanol-d₄) δ 8.63 (d, J=3.6 Hz, 1H), 7.84 (t, J=1.7 Hz, 1H), 7.51 (dd, J=4.5, 1.9 Hz, 1H), 7.18 (d, J=3.5 Hz, 1H), 6.04-5.88 (m, 1H), 4.99 (dd, J=8.5, 3.2 Hz, 1H), 4.36-4.21 (m, 2H), 3.67-3.42 (m, 2H), 3.04 (d, J=5.4 Hz, 2H), 2.87 (t, J=8.3 Hz, 2H), 2.69 (q, J=9.7, 8.0 Hz, 2H), 2.58 (q, J=7.2 Hz, 2H), 2.35 (s, 3H), 1.66 (dd, J=7.1, 3.2 Hz, 3H), 1.47 (td, J=7.0, 2.0 Hz, 3H), 1.12 (t, J=7.3 Hz, 3H), 0.86 (dd, J=7.2, 5.7 Hz, 3H). LC-MS: 533 [M+H]⁺.

### Example 259: Synthesis of 4-ethoxy-6-(1-(7-(2-(ethyl(methyl)amino)ethyl)-1-oxo-5-(2,2,2-trifluoro-1-hydroxyethyl)-3,4-dihydroisoquinolin-2(1H)-yl)ethyl)nicotinonitrile (259)

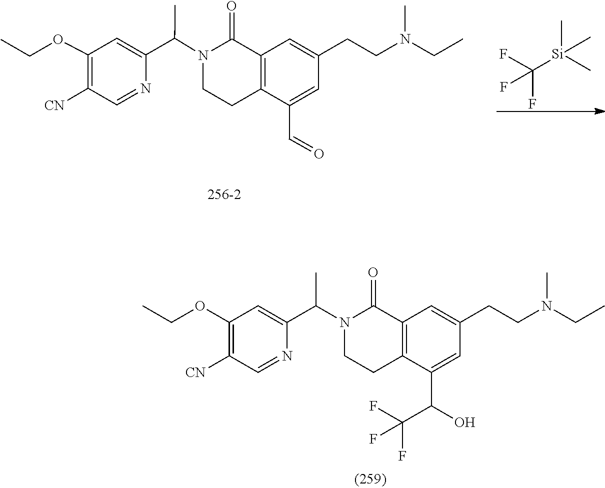

To the solution of compound (256-2) (58 mg, 0.133 mmol) in DMF (1.2 mL) was added trimethyl(trifluoromethyl)silane (0.040 mL, 0.267 mmol) and K₂CO₃ (1.845 mg, 0.013 mmol). Then the mixture was stirred at RT for 4 hrs. Then 2N HCl (0.4 mL) was added and further stirred for 2 hrs. The reaction was quenched with NaHCO₃ (aq) and extracted with EtOAc (30 mL×3). The combined organic layers were washed with brine (15 mL×2), dried over Na₂SO₄, under vacuum and purified by Flash Chromatography (CH₂Cl₂:MeOH=0-10%) to give the compound 4-ethoxy-6-(1-(7-(2-(ethyl(methyl)amino)ethyl)-1-oxo-5-(2,2,2-trifluoro-1-hydroxyethyl)-3,4-dihydroisoquinolin-2(1H)-yl)ethyl)nicotinonitrile (259) as a white solid. ¹H NMR (400 MHz, Methanol-d4) δ ppm 8.63 (d, J=6.4 Hz, 1H), 7.91 (d, J=2.3 Hz, 1H), 7.67 (s, 1H), 7.18 (s, 1H), 5.97 (dd, J=7.2, 2.0 Hz, 1H), 5.33 (dd, J=6.7, 1.7 Hz, 1H), 4.37-4.23 (m, 2H), 3.67-3.54 (m, 1H), 3.48 (dd, J=3.4, 1.8 Hz, 1H), 3.01 (d, J=6.5 Hz, 1H), 2.87 (t, J=8.1 Hz, 3H), 2.66 (d, J=9.4 Hz, 2H), 2.54 (t, J=7.2 Hz, 2H), 2.33 (d, J=1.8 Hz, 3H), 1.66 (dd, J=7.2, 5.1 Hz, 3H), 1.46 (td, J=7.0, 2.4 Hz, 3H), 1.11 (t, J=7.2 Hz, 3H). LC-MS: 505 [M+H]⁺.

Following a similar procedure to that of Example 259 (unless otherwise specified). The following compounds were prepared from their corresponding precursors:

| Ex. | Name/Structure | ¹HNMR/LC-MS | Precursors |
|---|---|---|---|
| 260 | 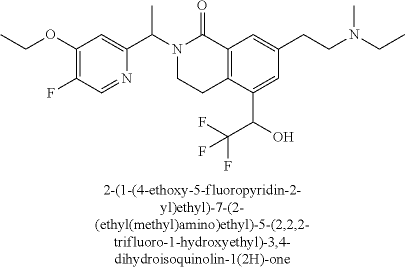 [SMILES: CCOc1cc(C(C)N2CCc3c(cc(CCN(C)CC)cc3C(O)C(F)(F)F)C2=O)ncc1F] | ¹H NMR (400 MHz, Methanol-d₄) δ 8.24 (dd, J = 5.9, 3.3 Hz, 1H), 7.94 (d, J = 1.7 Hz, 1H), 7.69 (s, 1H), 7.14 (d, J = 6.8 Hz, 1H), 5.98 (q, J = 7.1 Hz, 1H), 5.33 (tt, J = 6.9, 3.4 Hz, 1H), 4.25-4.14 (m, 2H), 3.61-3.32 (m, 2H), 3.10- 2.77 (m, 6H), 2.70 (q, J = 7.2 Hz, 2H), 1.63 (t, J = 6.8 Hz, 3H), 1.43 (td, J = 7.0, 2.4 Hz, 3H), 1.16 (t, J = 7.2 Hz, 3H). LC-MS: [M + H]⁺ = 498. | Compound (256- 2) was replaced with an analogue synthesized via example 156 using an analogue of compound 111- 1 synthesized via example 111 using intermediate (G5) in place of intermediate (G1) |

### Example 261 and Example 262: Synthesis of (R)-6-(1-(5-(1,3-Dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile (261) and (S)-6-(1-(5-(1,3-Dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile (262)

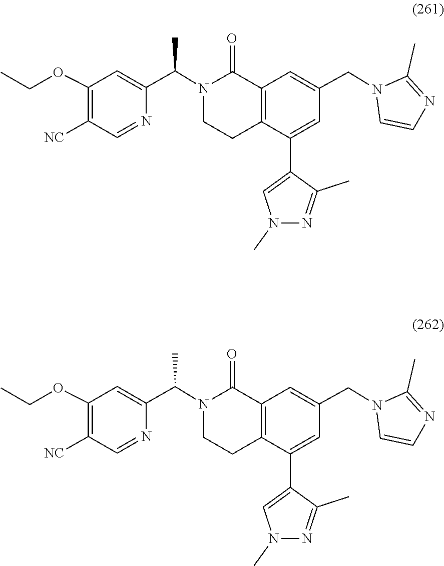 [SMILES: CCOc1cc([C@@H](C)N2CCc3c(cc(Cn4ccnc4C)cc3-c3cn(C)nc3C)C2=O)ncc1C#N ; CCOc1cc([C@H](C)N2CCc3c(cc(Cn4ccnc4C)cc3-c3cn(C)nc3C)C2=O)ncc1C#N]

Step 1: To a solution of 6-(1-(5-bromo-7-(hydroxymethyl)-1-oxo-3,4-dihydroisoquinolin-2(1H)-yl)ethyl)-4-ethoxynicotinonitrile (C1-3) (1.0 g, 2.32 mmol) in dioxane (5 mL) was added (1,3-dimethyl-1H-pyrazol-4-yl)boronic acid (650 mg, 4.65 mmol, 2 eq.), PdCl₂(dppf) (170 mg, 0.23 mmol, 0.1 eq.) and Na₂CO₃ (739 mg, 6.97 mmol, 3 eq.). The resulting mixture was stirred at 100° C. for 12 hours. Water was added and the mixture was extracted with ethyl acetate (100 mL×3). The combined organic layers were dried over anhydrous Na₂SO₄ and then concentrated to give the crude product. The crude product was purified by flash chromatography on silica gel (DCM:MeOH=10:1) to give 6-(1-(5-(1,3-Dimethyl-1H-pyrazol-4-yl)-7-(hydroxymethyl)-1-oxo-3,4-dihydroisoquinolin-2(1H)-yl)ethyl)-4-ethoxynicotinonitrile (300 mg, 29% yield). (ESI) m/z 446.1 [M+H]⁺.

Step 2: To a solution of 6-(1-(5-(1,3-dimethyl-1H-pyrazol-4-yl)-7-(hydroxymethyl)-1-oxo-3,4-dihydroisoquinolin-2(1H)-yl)ethyl)-4-ethoxynicotinonitrile (From step 1, 240 mg, 0.54 mmol) in DCM (3 mL) was added SOCl₂ (641 mg, 5.39 mmol, 10 eq.) at 0° C. The reaction mixture was stirred at 0° C. for 2 hours. Solvent was removed under vacuum. The crude product, 6-(1-(7-(chloromethyl)-5-(1,3-dimethyl-1H-pyrazol-4-yl)-1-oxo-3,4-dihydroisoquinolin-2(1H)-yl)ethyl)-4-ethoxynicotinonitrile, was obtained (250 mg, 100% yield) and used in next step without further purification. LCMS (ESI) m/z 464.1 [M+H]⁺.

Step 3: To a solution of 6-(1-(7-(chloromethyl)-5-(1,3-dimethyl-1H-pyrazol-4-yl)-1-oxo-3,4-dihydroisoquinolin-2(1H)-yl)ethyl)-4-ethoxynicotinonitrile (From step 2, 250 mg, 0.54 mmol) in DMF (5 mL) was added 2-methyl-1H-imidazole (48.7 mg, 0.59 mmol, 1.1 eq.) and K₂CO₃ (186 mg, 1.35 mmol, 2.5 eq.). The reaction mixture was stirred at 90° C. for 24 hours. Solvent was removed under reduced pressure to give the crude product. The crude product was purified first by flash chromatography on silica gel (DCM:MeOH=10:1) to give the 6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile racemic mixture. The racemic mixture was submitted to SFC (Column: (R,R)-WHELKO1, 5 um, 21.2 mm*250 mm, Condition: EtOH+0.5% NH₄OH) to give Examples (261) and Example (262).

Example (261): ¹H NMR (400 MHz, CD₃OD) δ 8.61 (s, 1H), 7.78 (d, J=2.0 Hz, 1H), 7.52 (s, 1H), 7.17 (s, 1H), 7.13 (d, J=2.0 Hz, 1H), 7.09 (d, J=1.5 Hz, 1H), 6.86 (d, J=1.4 Hz, 1H), 5.95 (d, J=7.1 Hz, 1H), 5.24 (s, 2H), 4.30 (q, J=7.0 Hz, 2H), 3.84 (s, 3H), 3.63-3.39 (m, 2H), 2.83 (td, J=5.2, 4.3, 1.9 Hz, 2H), 2.31 (s, 3H), 2.07 (s, 3H), 1.64 (d, J=7.1 Hz, 3H), 1.46 (t, J=7.0 Hz, 3H). LCMS: (ESI) m/z 510.2 [M+H]⁺.
Example (262): ¹H NMR (400 MHz, CD₃OD) δ 8.61 (s, 1H), 7.78 (s, 1H), 7.52 (s, 1H), 7.17 (s, 1H), 7.15-7.10 (m, 1H), 7.09 (d, J=1.5 Hz, 1H), 6.86 (d, J=1.4 Hz, 1H), 5.95 (d, J=7.1 Hz, 1H), 5.24 (s, 2H), 4.30 (q, J=7.0 Hz, 2H), 3.84 (s, 3H), 3.66-3.40 (m, 2H), 2.83 (dd, J=7.5, 3.3 Hz, 2H), 2.31 (s, 3H), 2.07 (s, 3H), 1.64 (d, J=7.1 Hz, 3H), 1.46 (t, J=7.0 Hz, 3H). LCMS: (ESI) m/z 510.2 [M+H]⁺.

### Example 263: Synthesis of 6-((5-(1,3-Dimethyl-1H-pyrazol-4-yl)-7-((2-methyl-1H-imidazol-1-yl)methyl)-1-oxo-3,4-dihydroisoquinolin-2(1H)-yl)methyl)-4-ethoxynicotinonitrile (263)

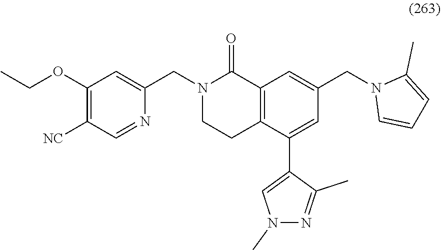 [SMILES: CCOc1cc(CN2CCc3c(cc(Cn4cccc4C)cc3-c3cn(C)nc3C)C2=O)ncc1C#N]

To a solution of 6-((5-bromo-7-((2-methyl-1H-imidazol-1-yl)methyl)-1-oxo-3,4-dihydroisoquinolin-2(1H)-yl)methyl)-4-ethoxynicotinonitrile (C2) (250 mg, 0.52 mmol) and 1,3-dimethyl-4-(4,4,5,5-tetramethyl-1,3,2-dioxaborolan-2-yl)-1H-pyrazole (230 mg, 1.04 mmol, 2.0 eq.) in dioxane (7.5 mL) and H₂O (1.5 mL) was added K₃PO₄ (222 mg, 1.04 mmol, 2.0 eq.) and DTBPFPdCl₂ (34 mg, 0.052 mmol, 0.1 eq.) at 20° C. under N₂. The mixture was stirred at 100° C. for 16 hours. H₂O (50 mL) was added and the mixture was then extracted with ethyl acetate (20 mL×5). The combined organic layers were dried over anhydrous Na₂SO₄, filtered and concentrated. The residue was purified by prep-HPLC (Column: Waters Xbridge 150*25*5 um; mobile phase: water (10 mM NH₄HCO₃)—CH₃CN; B %: 18%-48%; 10 minutes) to afford 6-((5-(1,3-Dimethyl-1H-pyrazol-4-yl)-7-((2-methyl-1H-imidazol-1-yl)methyl)-1-oxo-3,4-dihydroisoquinolin-2(1H)-yl)methyl)-4-ethoxynicotinonitrile (263) as a purple solid. ¹H NMR (400 MHz, CD₃OD) δ 8.60 (s, 1H), 7.79 (s, 1H), 7.55 (s, 1H), 7.17 (s, 1H), 7.14 (d, J=1.6 Hz, 1H), 7.09 (d, J=1.2 Hz, 1H), 6.87 (d, J=1.2 Hz, 1H), 5.25 (s, 2H), 4.87 (s, 2H), 4.30 (q, J=7.2 Hz, 2H), 3.86 (s, 3H), 3.64 (t, J=6.4 Hz, 2H), 2.94 (t, J=6.4 Hz, 2H), 2.32 (s, 3H), 2.08 (s, 3H), 1.47 (t, J=7.2 Hz, 3H). LCMS (ESI) m/z 496.3 [M+H]⁺.

### Example 264: Synthesis of (S)-6-(1-(5-(1,3-Dimethyl-1H-1,2,4-triazol-5-yl)-7-((2-methyl-1H-imidazol-1-yl)methyl)-1-oxo-3,4-dihydroisoquinolin-2(1H)-yl)ethyl)-4-ethoxynicotinonitrile (264)

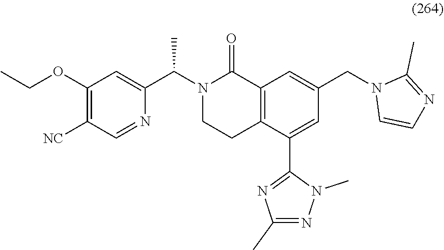 [SMILES: CCOc1cc([C@H](C)N2CCc3c(cc(Cn4ccnc4C)cc3-c3nc(C)nn3C)C2=O)ncc1C#N]

Step 1: To a solution of 6-(1-(5-bromo-7-((2-methyl-1H-imidazol-1-yl)methyl)-1-oxo-3,4-dihydroisoquinolin-2(1H)-yl)ethyl)-4-ethoxynicotinonitrile (C1) (400 mg, 0.81 mmol) in dioxane (6 mL) was added bis(pinacolato)diboron (616 mg, 2.43 mmol, 3 eq.), KOAc (238 mg, 2.43 mmol, 3 eq.), Pd(dppf)Cl₂ (66.1 mg, 0.081 mmol, 0.1 eq.). The reaction container was degassed thoroughly and refilled with nitrogen atmosphere. This resulting mixture was then heated at 110° C. overnight. The reaction mixture was diluted by ethyl acetate (5 mL) and filtered through Celite® to give a crude product. The crude product was purified by flash chromatography on silica gel (DCM:MeOH=100:3 to 100:5) to give 4-Ethoxy-6-(1-(7-((2-methyl-1H-imidazol-1-yl)methyl)-1-oxo-5-(4,4,5,5-tetramethyl-1,3,2-dioxaborolan-2-yl)-3,4-dihydroisoquinolin-2(1H)-yl)ethyl)nicotinonitrile (320 mg, 73% yield, contains small amount of de-boron starting material). LCMS (ESI) m/z 542 [M+H]⁺.

Step 2: To a solution of 4-ethoxy-6-(1-(7-((2-methyl-1H-imidazol-1-yl)methyl)-1-oxo-5-(4,4,5,5-tetramethyl-1,3,2-dioxaborolan-2-yl)-3,4-dihydroisoquinolin-2(1H)-yl)ethyl)nicotinonitrile (From step 1, 320 mg, 0.59 mmol) in dioxane (2 mL) and water (0.5 mL) was added 5-bromo-1,3-dimethyl-1H-1,2,4-triazole (312 mg, 1.77 mmol, 3 eq.), Na₂CO₃ (313 mg, 2.95 mmol, 5 eq.), Pd(dtbpf)Cl₂ (38.5 mg, 0.059 mmol, 0.1 eq.) under argon atmosphere. The reaction mixture was heated at 110° C. overnight. Solvent was removed under reduced pressure to give the crude product. The crude product was first purified by flash chromatography on silica gel (DCM:MeOH=100:5 to 100:8) to give a racemic mixture with residual palladium catalyst, which was further purified by preparative HPLC (Column: Sunfire C18, 5 um, 30 mm*150 mm, Mobile phase: A: H₂O (0.05% FA), B: ACN (0.05% FA)) to give pure product. The pure racemic product was then separated by SFC (Column: (R,R)-WHELKO1, 5 um, 4.6 mm*250 mm, Condition: EtOH+0.5% NH₄OH) to give (S)-6-(1-(5-(1,3-Dimethyl-1H-1,2,4-triazol-5-yl)-7-((2-methyl-1H-imidazol-1-yl)methyl)-1-oxo-3,4-dihydroisoquinolin-2(1H)-yl)ethyl)-4-ethoxynicotinonitrile (264) (the 2^{nd} peak on the prep SFC) together with its enantiomer. ¹H NMR (400 MHz, CD₃OD) δ 8.61 (s, 1H), 7.97 (d, J=2.0 Hz, 1H), 7.38 (d, J=2.0 Hz, 1H), 7.18 (s, 1H), 7.11 (d, J=1.5 Hz, 1H), 6.88 (d, J=1.4 Hz, 1H), 5.96 (d, J=7.1 Hz, 1H), 5.30 (s, 2H), 4.30 (q, J=7.0 Hz, 2H), 3.67 (s, 3H), 3.64-3.44 (m, 2H), 2.76 (m, 2H), 2.34 (d, J=14.5 Hz, 6H), 1.64 (d, J=7.1 Hz, 3H), 1.47 (t, J=7.0 Hz, 3H). LCMS (ESI) m/z 511.1 [M+H]+.

### Example 265: Synthesis of (S)-6-(1-(5-(3,5-Dimethyl-1H-1,2,4-triazol-1-yl)-7-((2-methyl-1H-imidazol-1-yl)methyl)-1-oxo-3,4-dihydroisoquinolin-2(1H)-yl)ethyl)-4-ethoxynicotinonitrile (265)

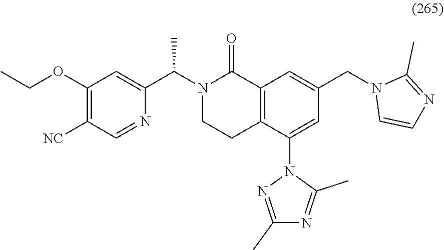 [SMILES: CCOc1cc([C@H](C)N2CCc3c(cc(Cn4ccnc4C)cc3-n3nc(C)nc3C)C2=O)ncc1C#N]

Step 1: A mixture of 6-(1-(5-bromo-7-((2-methyl-1H-imidazol-1-yl)methyl)-1-oxo-3,4-dihydroisoquinolin-2(1H)-yl)ethyl)-4-ethoxynicotinonitrile (C1) (1.00 g, 2.02 mmol), tert-butyl hydrazinecarboxylate (802 mg, 6.07 mmol, 3 eq.), Pd(OAc)₂ (45 mg, 0.20 mmol) and tri-tert-butylphosphonium tetrafluoroborate (117 mg, 0.41 mmol) in toluene (8 mL) was stirred at 110° C. under nitrogen atmosphere for 10 hours. The mixture was diluted with water, extracted with DCM. The organic layer was dried over anhydrous MgSO₄, filtered and concentrated in vacuum. The residue was purified by flash chromatography on silica gel (0-5% methanol in DCM containing 0.1% Et₃N) to afford a brown syrup (200 mg, 18% yield). ¹H NMR (400 MHz, CDCl₃) δ 8.60 (s, 1H), 7.52 (m, 1H), 7.09 (s, 1H), 6.96 (s, 1H), 6.89 (d, J=1.7 Hz, 1H), 6.71 (s, 1H), 6.34 (s, 1H), 6.04 (q, J=7.0 Hz, 1H), 5.65 (d, J=2.5 Hz, 1H), 5.07 (s, 2H), 4.20 (q, J=7.0 Hz, 2H), 3.69-3.38 (m, 2H), 3.18-3.01 (m, 1H), 2.83-2.60 (m, 2H), 2.55 (s, 3H), 1.62 (d, J=7.1 Hz, 3H), 1.50 (d, J=7.0 Hz, 3H), 1.46 (d, J=8.6 Hz, 9H). LC-MS: (ESI) m/z 546.3 [M+1]⁺.

Step 2: To a solution of tert-butyl 2-(2-(1-(5-cyano-4-ethoxypyridin-2-yl)ethyl)-7-((2-methyl-1H-imidazol-1-yl)methyl)-1-oxo-1,2,3,4-tetrahydroisoquinolin-5-yl)hydrazine-1-carboxylate (From step 1, 150 mg, 0.28 mmol) in DCM (4 mL) was added trifluoroacetic acid (1 mL, 12.98 mmol) and the reaction mixture was stirred at room temperature for 30 minutes. The mixture was concentrated in vacuum to give a residue. Water (10 mL) was added to the residue and pH of the solution was adjusted to 8. The mixture was extracted with DCM (50 mL) and the organic layer was dried over anhydrous MgSO₄ and was concentrated to give 4-ethoxy-6-(1-(5-hydrazinyl-7-((2-methyl-1H-imidazol-1-yl)methyl)-1-oxo-3,4-dihydroisoquinolin-2(1H)-yl)ethyl)nicotinonitrile (125 mg, quantitative yield) as a colorless oil that was carried over to the next step without further purification. LC-MS: (ESI) m/z 446.3 [M+1]⁺.z

Step 3: A solution of acetic acid (0.019 mL, 0.34 mmol), DIPEA (0.245 mL, 1.40 mmol), HATU (128 mg, 0.34 mmol) and acetimidamide hydrochloride (53.1 mg, 0.56 mmol) in DMF (3 mL) was stirred at room temperature for 1.5 hours. Then a solution of 4-ethoxy-6-(1-(5-hydrazinyl-7-((2-methyl-1H-imidazol-1-yl)methyl)-1-oxo-3,4-dihydroisoquinolin-2(1H)-yl)ethyl)nicotinonitrile (From step 2, 125 mg, 0.28 mmol) in DMF (2 mL) and acetic acid (0.16 mL, 2.81 mmol) was added to the mixture above and the new mixture was stirred at 80° C. for 3 hours. The mixture was concentrated and was purified by reverse CombiFlash (Column: C18 spherical 20-25 uM, 100 A, Condition: Water (0.1% FA in water)/ACN=9:1 to 6:4)) to afford the racemic product as a colorless syrup. It was further separated by SFC (Column: (R,R)-WHELKO1, 5 um, 21.2 mm*250 mm, Condition: EtOH+0.5% NH₄OH) to afford two isomers. The S-isomer is the second peak on SFC. ¹H NMR (400 MHz, CD₃OD) δ 8.60 (s, 1H), 7.96 (d, J=1.9 Hz, 1H), 7.36 (d, J=1.9 Hz, 1H), 7.18 (s, 1H), 7.11 (d, J=1.4 Hz, 1H), 6.88 (d, J=1.5 Hz, 1H), 5.94 (d, J=7.1 Hz, 1H), 5.30 (s, 2H), 4.30 (q, J=7.0 Hz, 2H), 3.65-3.45 (m, 2H), 2.66 (dq, J=6.4, 4.0, 2.9 Hz, 2H), 2.33 (d, J=7.4 Hz, 6H), 2.27 (s, 3H), 1.64 (d, J=7.1 Hz, 3H), 1.47 (t, J=7.0 Hz, 3H). LCMS: (ESI) m/z 511.2 [M+1]⁺.

### Example 266 and Example 267: Synthesis of 4-Ethoxy-6-((S)-1-(7-((2-methyl-1H-imidazol-1-yl)methyl)-5-((S)-1-methylpyrrolidin-2-yl)-1-oxo-3,4-dihydroisoquinolin-2(1H)-yl)ethyl)nicotinonitrile (266) and 4-ethoxy-6-((S)-1-(7-((2-methyl-1H-imidazol-1-yl)methyl)-5-((R)-1-methylpyrrolidin-2-yl)-1-oxo-3,4-dihydroisoquinolin-2(1H)-yl)ethyl)nicotinonitrile (267)

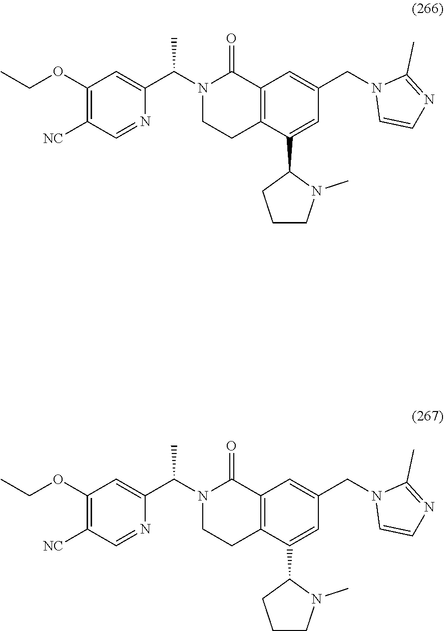 [SMILES: CCOc1cc([C@H](C)N2CCc3c(cc(Cn4ccnc4C)cc3[C@@H]3CCCN3C)C2=O)ncc1C#N ; CCOc1cc([C@H](C)N2CCc3c(cc(Cn4ccnc4C)cc3[C@H]3CCCN3C)C2=O)ncc1C#N]

Step 1: To a solution of 6-(1-(5-bromo-7-((2-methyl-1H-imidazol-1-yl)methyl)-1-oxo-3,4-dihydroisoquinolin-2(1H)-yl)ethyl)-4-ethoxynicotinonitrile (C1) (4.0 g, 8.1 mmol), (tert-butoxycarbonyl)proline (2.1 g, 10 mmol, 1.25 eq.), Ir[dF(CF₃)ppy]₂(dtbpy)(PF₆) (45 mg, 0.040 mmol, 0.005 eq.), NiCl₂.glyme (178 mg, 0.8 mmol, 0.1 eq.) and dtbbpy (239 mg, 0.8 mmol, 0.1 eq.) in DMF (50 mL) was added Cs₂CO₃ (4 g, 12.14 mmol, 1.5 eq.). The reaction mixture was degassed by bubbling nitrogen stream and was then irradiated with 34 W blue LED lamps (at approximately 2 cm away from the light source to keep the reaction temperature at 25° C. for 16 hours. The reaction mixture was diluted with water (50 mL), extracted with ethyl acetate (50 mL×3). The combined organic layers was dried over anhydrous Na₂SO₄, filtered and concentrated under reduced pressure to give a residue. The residue was purified by prep-HPLC (Column: Phenomenex Synergi Max-RP 250*50 mm*10 um, condition: water (0.225% FA)-ACN) to afford tert-butyl 2-(2-(1-(5-cyano-4-ethoxypyridin-2-yl)ethyl)-7-((2-methyl-1H-imidazol-1-yl)methyl)-1-oxo-1,2,3,4-tetrahydroisoquinolin-5-yl)pyrrolidine-1-carboxylate (440 mg, 9% yield) as a yellow oil. LCMS: MS (ESI) m/z 585.3, [M+H]⁺.

Step 2: To a solution of tert-butyl 2-(2-(1-(5-cyano-4-ethoxypyridin-2-yl)ethyl)-7-((2-methyl-1H-imidazol-1-yl)methyl)-1-oxo-1,2,3,4-tetrahydroisoquinolin-5-yl)pyrrolidine-1-carboxylate (From step 1, 330 mg, 0.57 mmol) in DCM (5 mL) were added trifluoroacetic acid (1 mL) at 25° C. Then the reaction mixture was stirred at 25° C. for 2 hours. To the reaction mixture was added saturated NaHCO₃ solution (50 mL) and the mixture was extracted with CHCl₃/ⁱPrOH (30 mL×5, V/V=3/1). The combined organic phases were dried over anhydrous Na₂SO₄, filtered and concentrated to afford 4-Ethoxy-6-(1-(7-((2-methyl-1H-imidazol-1-yl)methyl)-1-oxo-5-(pyrrolidin-2-yl)-3,4-dihydroisoquinolin-2(1H)-yl)ethyl)nicotinonitrile (300 mg, 80% purity) as a yellow oil. LCMS (ESI) m/z 485.2 [M+H]⁺.

Step 3: To a solution of 4-ethoxy-6-(1-(7-((2-methyl-1H-imidazol-1-yl)methyl)-1-oxo-5-(pyrrolidin-2-yl)-3,4-dihydroisoquinolin-2(1H)-yl)ethyl)nicotinonitrile (From step 2, 260 mg, 0.54 mmol) and HCHO (37% in H₂O, 218 mg) in EtOH (6 mL) was added NaBH₃CN (169 mg, 2.68 mmol, 5.0 eq.) at 25° C. Then the reaction mixture was stirred at 25° C. for 16 hours. To the reaction mixture was added saturated NH₄Cl solution (20 mL) and the mixture was extracted with CHCl₃/ⁱPrOH (20 mL×5, V/V=3/1). The combined organic phases were dried over anhydrous Na₂SO₄, filtered and concentrated. The residue was purified by prep-HPLC (Neutral) and lyophilized to afford the crude compound (70 mg, 93% purity) as a white solid. The crude compound was separated by chiral SFC (Column: DAICEL CHIRALCEL OD (250 mm*30 mm, 10 um); Gradient Time: 6.7 minutes, 90 minutes, Condition: 0.1% NH₃.H₂O, IPA) to give 4-ethoxy-6-((S)-1-(7-((2-methyl-1H-imidazol-1-yl)methyl)-5-((S)-1-methylpyrrolidin-2-yl)-1-oxo-3,4-dihydroisoquinolin-2(1H)-yl)ethyl)nicotinonitrile (Peak 3, 14.19 mg, 20.0% yield) and 4-ethoxy-6-((S)-1-(7-((2-methyl-1H-imidazol-1-yl)methyl)-5-((R)-1-methylpyrrolidin-2-yl)-1-oxo-3,4-dihydroisoquinolin-2(1H)-yl)ethyl)nicotinonitrile (Peak 4, 7.44 mg, 10.6% yield). The stereochemistry at the chiral center adjacent to pyrrolidine nitrogen on both compounds are arbitrarily assigned.

Example 266: ¹H NMR (400 MHz, CD₃OD) δ 8.62 (s, 1H), 7.53 (s, 1H), 7.49 (s, 1H), 7.15 (s, 1H), 6.87 (d, J=3.6 Hz, 2H), 5.93 (q, J=7.2 Hz, 1H), 5.30 (d, J=5.6 Hz, 2H), 4.32-4.26 (m, 2H), 3.61 (m, 1H), 3.52 (m, 1H), 3.33 (m, 1H), 3.25-3.21 (m, 1H), 2.98-2.95 (m, 2H), 2.33-2.31 (m, 4H), 2.14 (d, J=4.4 Hz, 3H), 2.11 (m, 1H), 1.84 (m, 2H), 1.65 (d, J=7.2 Hz, 3H), 1.47 (m, 1H), 1.46-1.44 (t, J=7.0 Hz, 3H). LCMS (ESI) m/z 499.3 [M+H]⁺.
Example 267: ¹H NMR (400 MHz, CD₃OD) δ 8.62 (s, 1H), 7.70 (d, J=1.2 Hz, 1H), 7.55 (s, 1H), 7.17 (s, 1H), 7.05 (d, J=1.2 Hz, 1H), 6.85 (d, J=1.2 Hz, 1H), 5.97-5.91 (q, J=6.8 Hz, 1H), 5.20 (m, 2H), 4.32 (m, 2H), 3.59-3.50 (m, 2H), 3.39 (m, 1H), 3.21 (m, 1H), 3.12-2.88 (m, 2H), 2.34-2.33 (m, 1H), 2.31 (s, 3H), 2.30-2.20 (m, 1H), 2.15 (d, J=10.6 Hz, 3H), 1.86-1.84 (m, 2H), 1.66 (d, J=6.8 Hz, 3H), 1.61-1.49 (m, 1H), 1.47-1.44 (m, 3H). LCMS (ESI) m/z 499.3 [M+H]⁺.

### Example 268: Synthesis of (S)-6-(1-(5-(1,3-Dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinamide (268)

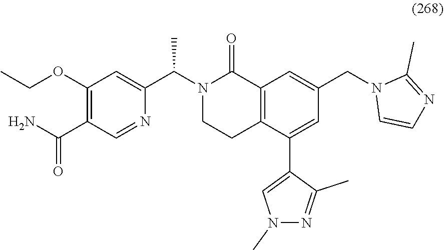 [SMILES: CCOc1cc([C@H](C)N2CCc3c(cc(Cn4ccnc4C)cc3-c3cn(C)nc3C)C2=O)ncc1C(N)=O]

To a stirred solution of 6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile (the racemic mixture from step 3 of Example 261, 20 mg, 0.039 mmol) in DMSO (1 mL) cooled in an ice bath was added H₂O₂ (30%, 0.04 mL) and K₂CO₃ (16.3 mg, 0.12 mmol, 33 eq.). The mixture was then allowed to warm up to room temperature. After 1 hour, distilled water and ethyl acetate were added to the mixture and the organic phase was separated, dried over anhydrous Na₂SO₄, filtered and the solvent removed under reduced pressure. The crude mixture was separated by SFC (Column: (R,R)-WHELKO1, 5 um, 21.2 mm*250 mm, Condition: MeOH+0.5% NH₄OH) to give (S)-6-(1-(5-(1,3-Dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinamide (268) as peak 2 (6.3 mg, 30% yield). ¹H NMR (400 MHz, CD₃OD) δ 8.88 (d, J=1.8 Hz, 1H), 8.53 (s, 1H), 7.80 (s, 1H), 7.52 (d, J=1.8 Hz, 1H), 7.25-7.07 (m, 3H), 6.95 (m, 1H), 5.99 (dd, J=7.0, 1.6 Hz, 1H), 5.26 (d, J=2.5 Hz, 2H), 4.33 (q, J=7.0 Hz, 2H), 3.84 (d, J=1.8 Hz, 3H), 3.65-3.47 (m, 2H), 2.82 (d, J=6.5 Hz, 1H), 2.33 (d, J=3.3 Hz, 3H), 2.07 (d, J=1.9 Hz, 3H), 1.66 (dd, J=7.2, 1.8 Hz, 3H), 1.49 (td, J=7.0, 1.8 Hz, 3H).

### Example 269: Synthesis of (S)-4-Ethoxy-6-(1-(7-((2-methyl-1H-imidazol-1-yl)methyl)-1-oxo-5-(6-oxo-4-(trifluoromethyl)-1,6-dihydropyridin-3-yl)-3,4-dihydroisoquinolin-2(1H)-yl)ethyl)nicotinonitrile (269)

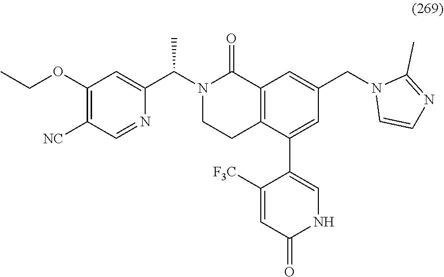 [SMILES: CCOc1cc([C@H](C)N2CCc3c(cc(Cn4ccnc4C)cc3-c3c[nH]c(=O)cc3C(F)(F)F)C2=O)ncc1C#N]

Step 1: 6-(1-(5-Bromo-7-((2-methyl-1H-imidazol-1-yl)methyl)-1-oxo-3,4-dihydroisoquinolin-2(1H)-yl)ethyl)-4-ethoxynicotinonitrile (C1), (500 mg) was separated by chiral SFC (column: DAICEL CHIRALCEL OD (250 mm*30 mm, 10 um); Condition: 0.05% DEA EtOH) to give the S-isomer as peak 2 (235 mg, 47% yield) as a yellow oil. ¹H NMR (400 MHz, CDCl₃) δ 8.60 (s, 1H), 7.89 (d, J=1.6 Hz, 1H), 7.30 (d, J=1.6 Hz, 1H), 6.99 (d, J=1.6 Hz, 1H), 6.96 (s, 1H), 6.85 (d, J=1.2 Hz, 1H), 6.04-5.99 (m, 1H), 5.04 (s, 2H), 4.23-4.18 (m, 2H), 3.62-3.55 (m, 2H), 3.04-3.02 (m, 1H), 2.93-2.90 (m, 1H), 2.35 (s, 3H), 1.64 (d, J=7.2 Hz, 3H), 1.51-1.47 (m, 3H). LCMS (ESI) m/z 494.0, [M+H]⁺. SFC: Rt=2.278 min, ee value >98%.

Step 2: To a solution of 4-(trifluoromethyl)pyridin-2(1H)-one (3.0 g, 18.3 mmol) in CH₃CN (10 mL) was added Br₂ (5.9 g, 36.8 mmol, 2.0 eq.). The reaction mixture was stirred at 80° C. for 16 hours and was then cooled to 25° C. The mixture was filtered, the solid was concentrated under reduce pressure to give 5-Bromo-4-(trifluoromethyl)pyridin-2(1H)-one (2.8 g, 61% yield) as a white solid. ¹H NMR (400 MHz, DMSO-d6) δ 8.03-8.01 (m, 1H), 8.02 (s, 1H), 8.00-7.93 (m, 1H), 6.88 (s, 1H). LCMS: (ESI) m/z 241.9 [M+H]⁺.

Step 3: To a solution of 5-bromo-4-(trifluoromethyl)pyridin-2(1H)-one (From step 2, 500 mg, 2.07 mmol) in THF (5 mL) was added t-BuOK (696 mg, 6.20 mmol, 3.0 eq.) and SEMCI (0.73 mL, 689 mg, 4.13 mmol, 2.0 eq.) at 0° C. The mixture was stirred at 25° C. for 16 hours. The mixture was diluted with H₂O (20 mL) and extracted with ethyl acetate (10 mL×3). The combined organic layers were dried over anhydrous Na₂SO₄, filtered and concentrated. The residue was purified by flash chromatography on silica gel (petroleum ether/ethyl acetate from 0% to 100%) to afford 5-Bromo-4-(trifluoromethyl)-1-((2-(trimethylsilyl)ethoxy)methyl)pyridin-2(1H)-one as a colorless oil. ¹H NMR (400 MHz, CDCl₃) δ 7.72 (s, 1H), 6.94 (s, 1H), 5.32 (s, 2H), 3.68-3.63 (m, 2H), 1.00-0.96 (m, 2H), 0.03 (s, 9H).

Step 4: Following a similar procedure to that of step 1 of Example 164, (S)-4-Ethoxy-6-(1-(7-((2-methyl-1H-imidazol-1-yl)methyl)-1-oxo-5-(4,4,5,5-tetramethyl-1,3,2-dioxaborolan-2-yl)-3,4-dihydroisoquinolin-2(1H)-yl)ethyl)nicotinonitrile was prepared from the (S) isomer of 6-(1-(5-Bromo-7-((2-methyl-1H-imidazol-1-yl)methyl)-1-oxo-3,4-dihydroisoquinolin-2(1H)-yl)ethyl)-4-ethoxynicotinonitrile (C1) from step 1. This crude product was carried over to the next step without further purification. LCMS (ESI) m/z 542.3 [M+H]⁺.

Step 5: Following a similar procedure to that of step 2 of Example 164, (S)-4-Ethoxy-6-(1-(7-((2-methyl-1H-imidazol-1-yl)methyl)-1-oxo-5-(6-oxo-4-(trifluoromethyl)-1-((2-(trimethylsilyl)ethoxy)methyl)-1,6-dihydropyridin-3-yl)-3,4-dihydroisoquinolin-2(1H)-yl)ethyl)nicotinonitrile was prepared from (S)-4-Ethoxy-6-(1-(7-((2-methyl-1H-imidazol-1-yl)methyl)-1-oxo-5-(4,4,5,5-tetramethyl-1,3,2-dioxaborolan-2-yl)-3,4-dihydroisoquinolin-2(1H)-yl)ethyl)nicotinonitrile (From step 4) and 5-Bromo-4-(trifluoromethyl)-1-((2-(trimethylsilyl)ethoxy)methyl)pyridin-2(1H)-one (From step 3). LCMS (ESI) m/z 707.3 [M+H]⁺.

Step 6: To a solution of (S)-4-ethoxy-6-(1-(7-((2-methyl-1H-imidazol-1-yl)methyl)-1-oxo-5-(6-oxo-4-(trifluoromethyl)-1-((2-(trimethylsilyl)ethoxy)methyl)-1,6-dihydropyridin-3-yl)-3,4-dihydroisoquinolin-2(1H)-yl)ethyl)nicotinonitrile (From step 5, 20 mg, 0.028 mmol) in DCM (1 mL) was added TFA (0.1 mL) at 25° C. The reaction mixture was stirred at 25° C. for 2 hours. To the reaction mixture was added saturated NaHCO₃ solution (2 mL) and the mixture was extracted with DCM (2 mL×3). The combined organic layers were dried over anhydrous Na₂SO₄, filtered and concentrated. The residue was purified by prep-HPLC (0.1% FA) and lyophilized to afford (S)-4-Ethoxy-6-(1-(7-((2-methyl-1H-imidazol-1-yl)methyl)-1-oxo-5-(6-oxo-4-(trifluoromethyl)-1,6-dihydropyridin-3-yl)-3,4-dihydroisoquinolin-2(1H)-yl)ethyl)nicotinonitrile (269) as a brown solid. ¹H NMR (400 MHz, CD₃OD) δ 8.60 (s, 1H), 7.88 (s, 1H), 7.44 (d, J=14.0 Hz, 1H), 7.19-7.05 (m, 3H), 6.92 (d, J=3.2 Hz, 1H), 6.80 (s, 1H), 5.96-5.92 (m, 1H), 5.25 (s, 2H), 4.33-4.27 (m, 2H), 3.61-3.50 (m, 2H), 2.78-2.74 (m, 1H), 2.62-2.56 (m, 1H), 2.30 (s, 3H), 1.66-1.62 (m, 3H), 1.48-1.46 (m, 3H). LCMS (ESI) m/z 577.1 [M+H]⁺.

### Example 270: Synthesis of (S)-6-(1-(5-(3-((Dimethylamino)methyl)-1-methyl-1H-pyrazol-5-yl)-7-((2-methyl-1H-imidazol-1-yl)methyl)-1-oxo-3,4-dihydroisoquinolin-2(1H)-yl)ethyl)-4-ethoxynicotinonitrile (270)

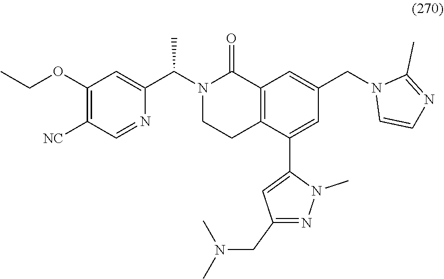 [SMILES: CCOc1cc([C@H](C)N2CCc3c(cc(Cn4ccnc4C)cc3-c3cc(CN(C)C)nn3C)C2=O)ncc1C#N]

Step 1: To a solution of ethyl 5-bromo-1-methyl-1H-pyrazole-3-carboxylate (2.0 g, 8.58 mmol) in THF (20 mL) was added lithium borohydride solution (2.0 M in THF, 10.73 mL, 21.45 mmol, 2.5 eq.) at 0° C. under N₂ atmosphere. The resulting mixture was stirred at room temperature for 12 hours. 10 mL of 2M HCl solution was added into reaction mixture and the solvent was evaporated under vacuum to give crude product. The crude product was purified by flash chromatography on silica gel (hexanes/ethyl acetate=2/1) to give (5-bromo-1-methyl-1H-pyrazol-3-yl)methanol (1.2 g, 73.2% yield) as a colorless oil. LCMS (ESI) m/z 191.1 [M+H]⁺.

Step 2: To a solution of (5-bromo-1-methyl-1H-pyrazol-3-yl)methanol (From step 1, 200 mg, 1.047 mmol) in DCM (5 mL) was added MnO₂ (910 mg, 10.47 mmol). The mixture was stirred at 40° C. for 2 hours. The mixture was filtered and the filtrate was concentrated to afford 5-bromo-1-methyl-1H-pyrazole-3-carbaldehyde (190 mg, 96% yield) as a colorless oil. The crude product was used for the next step directly. LCMS (ESI) m/z 189.1 [M+H]⁺.

Step 3: A suspension of 5-bromo-1-methyl-1H-pyrazole-3-carbaldehyde (From step 2, 190 mg, 1.005 mmol), PdCl₂(dppf).CH₂Cl₂ adduct (57.5 mg, 0.070 mmol), 4,4,4′,4′,5,5,5′,5′-octamethyl-2,2′-bi(1,3,2-dioxaborolane) (511 mg, 2.010 mmol) and potassium acetate (148 mg, 1.508 mmol) in dioxane (5 mL) was stirred at 80° C. under nitrogen atmosphere for 2 hours. The reaction mixture was filtered through a paddle of Celite and the solvent was removed under vacuum to give 1-methyl-5-(4,4,5,5-tetramethyl-1,3,2-dioxaborolan-2-yl)-1H-pyrazole-3-carbaldehyde (191 mg, 80.9% yield), which was used for the next step without further purification. LCMS (ESI) m/z 155.1 [M+H]⁺.

Step 4: To a suspension of 6-(1-(5-bromo-7-((2-methyl-1H-imidazol-1-yl)methyl)-1-oxo-3,4-dihydroisoquinolin-2(1H)-yl)ethyl)-4-ethoxynicotinonitrile (C1) (200 mg, 0.405 mmol), 1-methyl-5-(4,4,5,5-tetramethyl-1,3,2-dioxaborolan-2-yl)-1H-pyrazole-3-carbaldehyde (From step 3,191 mg, 0.809 mmol), K₃PO₄ (172 mg, 0.809 mmol) and Pd(dtbpf)Cl₂ (13.2 mg, 0.020 mmol) in dioxane (5 mL) was added water (1.5 mL). The mixture was stirred at 80° C. under nitrogen protection for 1 hour. The mixture was diluted with water, extracted with DCM. The organic layer was dried over anhydrous magnesium sulfate, filtered and concentrated. The residue was purified by flash chromatography on silica gel (Eluted with methanol in DCM (0-5%, 0.05% TEA)) to afford 4-Ethoxy-6-(1-(5-(3-formyl-1-methyl-1H-pyrazol-5-yl)-7-((2-methyl-1H-imidazol-1-yl)methyl)-1-oxo-3,4-dihydroisoquinolin-2(1H)-yl)ethyl)nicotinonitrile (210 mg, 99% yield) as a brown solid. LCMS (ESI) m/z 524.2 [M+H]⁺.

Step 5: To a solution of 4-ethoxy-6-(1-(5-(3-formyl-1-methyl-1H-pyrazol-5-yl)-7-((2-methyl-1H-imidazol-1-yl)methyl)-1-oxo-3,4-dihydroisoquinolin-2(1H)-yl)ethyl)nicotinonitrile (From step 4, 170 mg, 0.325 mmol), dimethylamine in THF (0.487 mL, 0.974 mmol) in methanol (4 mL) was added acetic acid (0.019 mL, 0.325 mmol), the mixture was stirred at room temperature for 1 hour, then NaBH₃CN (40.8 mg, 0.649 mmol) was added, the mixture was stirred at room temperature for another 15 hours. Solvent was removed under vacuum and the crude product was purified by acidic Prep-HPLC(Column: Sunfire C18, 5 um, 30 mm*150 mm, Mobile phase: A: H₂O (0.05% FA), B: ACN (0.05% FA)). Desired fraction was collected and lyophilized to afford 100 mg desired target as white powder, which was further separated by SFC (Column: (R,R)-WHELKO1, 5 um, 21.2 mm*250 mm, Condition: MeOH+0.5% NH₄OH) to obtain (S)-6-(1-(5-(3-((Dimethylamino)methyl)-1-methyl-1H-pyrazol-5-yl)-7-((2-methyl-1H-imidazol-1-yl)methyl)-1-oxo-3,4-dihydroisoquinolin-2(1H)-yl)ethyl)-4-ethoxynicotinonitrile (270) as the second peak. ¹H NMR (400 MHz, CD₃OD) δ 8.60 (s, 1H), 8.40 (s, 2H), 7.94 (d, J=2.0 Hz, 1H), 7.31 (d, J=2.0 Hz, 1H), 7.24-7.10 (m, 2H), 7.00 (d, J=1.6 Hz, 1H), 6.44 (s, 1H), 5.95 (q, J=7.1 Hz, 1H), 5.33 (s, 2H), 4.41-4.26 (m, 2H), 4.20 (s, 2H), 3.66 (s, 3H), 3.63-3.44 (m, 2H), 2.82 (s, 6H), 2.76 (t, J=6.5 Hz, 2H), 2.39 (s, 3H), 1.65 (d, J=7.1 Hz, 3H), 1.47 (t, J=7.0 Hz, 3H). LCMS (ESI) m/z 553.3 [M+H]⁺.

Following a similar procedure to that of Example 163 and Example 164 (unless otherwise specified). The following compounds were prepared from their corresponding precursors.

| Ex. | Name/Structure | ¹H NMR/LCMS | Precursor 1 | Precursor 2 |
|---|---|---|---|---|
| 271 | 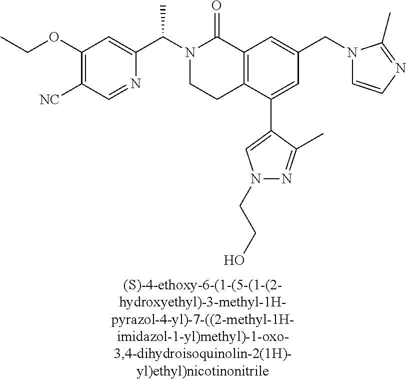 [SMILES: CCOc1cc([C@H](C)N2CCc3c(cc(Cn4ccnc4C)cc3-c3cn(CCO)nc3C)C2=O)ncc1C#N] | ¹H NMR (400 MHz, CD₃OD) δ 8.61 (s, 1H), 7.78 (d, J = 2.0 Hz, 1H), 7.58 (s, 1H), 7.18 (s, 1H), 7.14 (d, J = 2.0 Hz, 1H), 7.09 (d, J = 1.4 Hz, 1H), 6.86 (d, J = 1.4 Hz, 1H), 5.96 (q, J = 7.1 Hz, 1H), 5.24 (s, 2H), 4.30 (q, J = 7.0 Hz, 2H), 4.17 (t, J = 5.3 Hz, 2H), 3.87 (dd, J = 5.8, 4.9 Hz, 2H), 3.63-3.39 (m, 2H), 2.85 (q, J = 5.8 Hz, 2H), 2.31 (s, 3H), 2.08 (s, 3H), 1.64 (d, J = 7.1 Hz, 3H), 1.47 (t, J = 7.0 Hz, 3H). LCMS (ESI) m/z 540.2 [M + 1]⁺. | 6-(1-(5-bromo- 7-((2-methyl- 1H-imidazol-1- yl)methyl)-1- oxo-3,4- dihydroiso- quinolin- 2(1H)- yl)ethyl)-4- ethoxynico- tinonitrile (C1) | 2-(3-methyl- 4-(4,4,5,5- tetramethyl- 1,3,2- dioxaborolan- 2-yl)-1H- pyrazol-1- yl)ethan-1- ol |
| 272 | 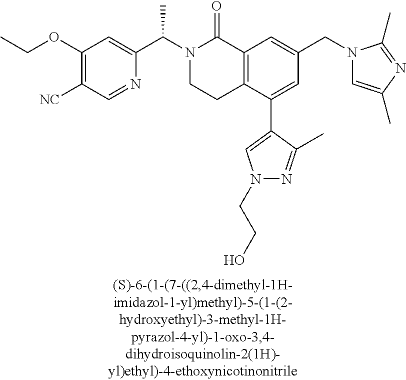 [SMILES: CCOc1cc([C@H](C)N2CCc3c(cc(Cn4cc(C)nc4C)cc3-c3cn(CCO)nc3C)C2=O)ncc1C#N] | ¹H NMR (400 MHz, CD₃OD) δ 8.61 (s, 1H), 7.77 (d, J = 2.0 Hz, 1H), 7.58 (s, 1H), 7.17 (s, 1H), 7.15 (d, J = 1.9 Hz, 1H), 6.78 (d, J = 2.0 Hz, 1H), 5.95 (d, J = 7.1 Hz, 1H), 5.16 (s, 2H), 4.31 (q, J = 7.0 Hz, 2H), 4.17 (t, J = 5.3 Hz, 2H), 3.87 (t, J = 5.3 Hz, 2H), 3.62-3.40 (m, 2H), 2.85 (q, J = 5.8 Hz, 2H), 2.28 (d, J = 1.7 Hz, 3H), 2.12 (s, 3H), 2.09 (s, 3H), 1.64 (d, J = 7.1 Hz, 3H), 1.47 (t, J = 7.0 Hz, 3H). LCMS (ESI) m/z 554.2 [M + 1]⁺. | 6-(1-(5-Bromo- 7-((2,4- dimethyl-1H- imidazol-1- yl)methyl)-1- oxo-3,4- dihydroiso- quinolin- 2(1H)- yl)ethyl)-4- ethoxynico- tinonitrile (C4) | 2-(3-methyl- 4-(4,4,5,5- tetramethyl- 1,3,2- dioxaborolan- 2-yl)-1H- pyrazol-1-yl) ethan-1-ol |
| 273 | 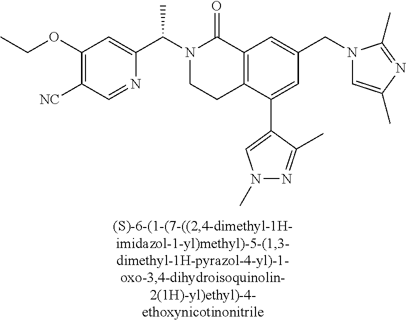 [SMILES: CCOc1cc([C@H](C)N2CCc3c(cc(Cn4cc(C)nc4C)cc3-c3cn(C)nc3C)C2=O)ncc1C#N] | ¹H NMR (400 MHz, CD₃OD) δ 8.61 (s, 1H), 7.76 (s, 1H), 7.52 (s, 1H), 7.17 (s, 1H), 7.13 (d, J = 1.9 Hz, 1H), 6.76 (s, 1H), 5.95 (q, J = 7.1 Hz, 1H), 5.15 (s, 2H), 4.30 (q, J = 7.0 Hz, 2H), 3.84 (d, J = 1.1 Hz, 3H), 3.49 (dq, J = 28.4, 6.2 Hz, 2H), 2.91-2.75 (m, 2H), 2.26 (d, J = 1.2 Hz, 3H), 2.11 (s, 3H), 2.07 (d, J = 1.1 Hz, 3H), 1.64 (d, J = 7.1 Hz, 3H), 1.46 (td, J = 7.0, 1.2 Hz, 3H). LCMS (ESI) m/z 524.3 [M + 1]⁺. | 6-(1-(7- (chloro- methyl)- 5-(1,3- dimethyl- 1H-pyrazol-4- yl)-1-oxo-3,4- dihydroiso- quinolin- 2(1H)- yl)ethyl)-4- ethoxynico- tinonitrile (From step 2 of Example 261) | 2,4-dimethyl- 1H-imidazole (Following the procedure of Example 261) |
| 274 | 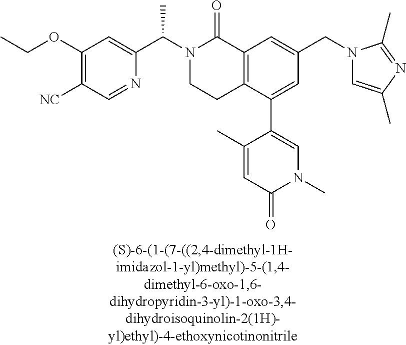 [SMILES: CCOc1cc([C@H](C)N2CCc3c(cc(Cn4cc(C)nc4C)cc3-c3cn(C)c(=O)cc3C)C2=O)ncc1C#N] | ¹H NMR (400 MHz, CD₃OD) δ 8.60 (s, 1H), 7.83 (t, J = 2.1 Hz, 1H), 7.46 (d, J = 14.3 Hz, 1H), 7.17 (d, J = 1.7 Hz, 2H), 6.76 (d, J = 1.1 Hz, 1H), 6.50 (dd, J = 4.8, 0.9 Hz, 1H), 5.95 (dd, J = 7.2, 4.5 Hz, 1H), 5.15 (s, 2H), 4.25-4.20 (m, 2H), 3.64-3.40 (m, 5H), 2.89-2.55 (m, 2H), 2.27 (s, 3H), 2.11 (d, J = 1.0 Hz, 3H), 1.91 (dd, J = 16.6, 1.0 Hz, 3H), 1.64 (d, J = 7.1 Hz, 3H), 1.47 (td, J = 7.0, 0.9 Hz, 3H). LCMS (ESI) m/z 551.2 [M + 1]⁺. | 6-(1-(7- (chloro- methyl)- 5-(1,4- dimethyl- 6-oxo-1,6- dihydro- pyridin-3- yl)-1-oxo- 3,4-dihydro- isoquinolin- 2(1H)- yl)ethyl)-4- ethoxynico- tinonitrile | 2,4-dimethyl- 1H-imidazole (Following the procedure of Example 261) |
| 275 | 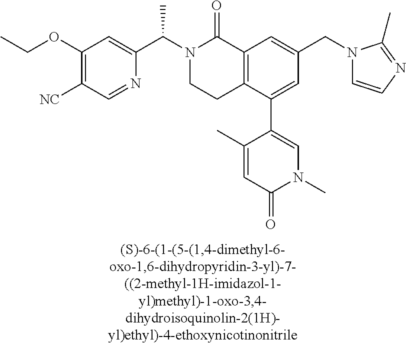 [SMILES: CCOc1cc([C@H](C)N2CCc3c(cc(Cn4ccnc4C)cc3-c3cn(C)c(=O)cc3C)C2=O)ncc1C#N] | ¹H NMR (400 MHz, CD₃OD) δ 8.60 (s, 1H), 7.84 (t, J = 2.1 Hz, 1H), 7.46 (d, J = 14.2 Hz, 1H), 7.17 (d, J = 1.8 Hz, 2H), 7.09 (d, J = 1.6 Hz, 1H), 6.90 (m, 1H), 6.50 (dd, J = 4.7, 1.0 Hz, 1H), 5.94 (td, J = 7.1, 4.5 Hz, 1H), 5.25 (s, 2H), 4.25 (m, 2H), 3.66-3.39 (m, 5H), 2.80-2.60 (m, 2H), 2.32 (d, J = 1.2 Hz, 3H), 1.91 (dd, J = 16.6, 1.0 Hz, 3H), 1.64 (d, J = 7.1 Hz, 3H), 1.46 (td, J = 7.0, 1.0 Hz, 3H). LCMS (ESI) m/z 537.2 [M + 1]⁺. | 6-(1-(7- (chloro- methyl)- 5-(1,4- dimethyl- 6-oxo-1,6- dihydro- pyridin-3- yl)-1-oxo- 3,4-dihydro- isoquinolin- 2(1H)- yl)ethyl)-4- ethoxynico- tinonitrile | 2-methyl- 1H- imidazole (Following the procedure of Example 261) |
| 276 | 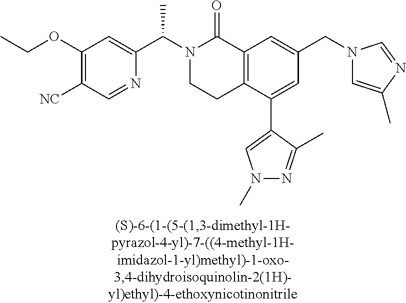 | ¹H NMR (400 MHz, CD₃OD) δ 8.61 (s, 1H), 7.85 (d, J = 1.9 Hz, 1H), 7.64 (d, J = 1.3 Hz, 1H), 7.52 (s, 1H), 7.26 (d, J = 2.0 Hz, 1H), 7.17 (s, 1H), 6.82 (t, J = 1.2 Hz, 1H), 5.96 (d, J = 7.1 Hz, 1H), 5.20 (s, 2H), 4.30 (q, J = 7.0 Hz, 2H), 3.85 (s, 3H), 3.61-3.39 (m, 2H), 2.82 (td, J = 8.2, 7.2, 3.6 Hz, 2H), 2.15 (d, J = 0.9 Hz, 3H), 2.08 (s, 3H), 1.64 (d, J = 7.1 Hz, 3H), 1.46 (t, J = 7.0 Hz, 3H). LCMS (ESI) m/z | 6-(1-(7- (chloro- methyl)- 5-(1,3- dimethyl- 1H-pyrazol- 4-yl)-1-oxo- 3,4-dihydro- isoquinolin- 2(1H)- yl)ethyl)-4- ethoxynico- tinonitrile (From step 2 of Example 261) | 4-methyl- 1H- imidazole |
| | | 510.2 [M + 1]⁺. | | |
| 277 | 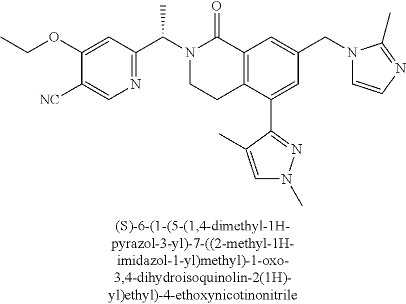 [SMILES: CCOc1cc([C@H](C)N2CCc3c(cc(Cn4ccnc4C)cc3-c3nn(C)cc3C)C2=O)ncc1C#N] | ¹H NMR (400 MHz, DMSO-d6) δ 8.75 (s, 1H), 7.73 (d, J = 1.6 Hz, 1H), 7.56 (s, 1H), 7.20 (s, 1H), 7.16 (dd, J = 4.8, 1.6 Hz, 2H), 6.77 (d, J = 1.0 Hz, 1H), 5.89 (q, J = 7.0 Hz, 1H), 5.23 (s, 2H), 4.30 (q, J = 7.0 Hz, 2H), 3.79 (s, 3H), 3.31-3.25 (m, 2H), 2.89-2.77 (m, 2H), 2.22 (s, 3H), 1.87 (s, 3H), 1.54 (d, J = 7.2 Hz, 3H), 1.35 (t, J = 7.0 Hz, 3H). LCMS (ESI) m/z 510.3 [M + 1]⁺. | 4-ethoxy-6-(1- (7-((2-methyl- 1H-imidazol- 1-yl)methyl)- 1-oxo-5- (4,4,5,5- tetramethyl- 1,3,2- dioxaborolan- 2-yl)-3,4- dihydroiso- quinolin- 2(1H)- yl)ethyl) nicotino- nitrile (From step 1 of Example 264) | 3-bromo-1,4- dimethyl-1H- pyrazole |
| 278 | 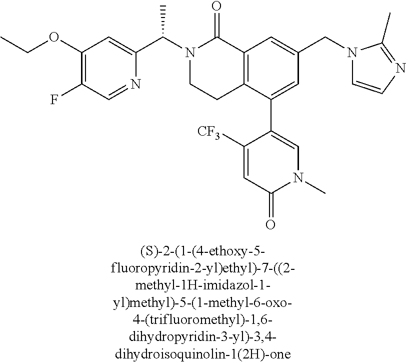 [SMILES: CCOc1cc([C@H](C)N2CCc3c(cc(Cn4ccnc4C)cc3-c3cn(C)c(=O)cc3C(F)(F)F)C2=O)ncc1F] | Separated by chiral SFC (column: DAICEL CHIRALCEL IC (250 mm*30 mm, 10 um); Gradient Time: 3.5 min, 60 min; Condition: 0.1% NH₃•H₂O, MeOH), 2^{nd} peak. ¹H NMR (400 MHz, CD₃OD) δ 8.22 (d, J = 3.2 Hz, 1H), 7.93 (s, 1H), 7.71 (d, J = 19.2 Hz, 1H), 7.20-7.11 (m, 3H), 6.96 (s, 1H), 6.92 (d, J = 4.0 Hz, 1H), 5.99-5.93 (m, 1H), 5.29 (s, 2H), 4.22-4.17 (m, 2H), 3.60 (d, J = 10.8 Hz, 3H), 3.44-3.41 (m, 2H), 2.88-2.68 (m, | 2-(1-(4- ethoxy-5- fluoro- pyridin-2- yl)ethyl)-7- ((2-methyl- 1H- imidazol-1- yl)methyl)- 5-(4,4,5,5- tetramethyl- 1,3,2- dioxaborolan- 2-yl)-3,4- dihydroiso- quinolin- 1(2H)-one | 5-bromo-1- methyl-4- (trifluoro- methyl) pyridin- 2(1H)-one |
| | | 1H), 2.60-2.55 (m, | | |
| | | 1H), 2.35 (s, 3H), | | |
| | | 1.62-1.59 (m, 3H), | | |
| | | 1.45-1.40 (m, 3H). | | |
| | | LCMS (ESI) m/z | | |
| | | 584.2 [M + H]⁺. | | |
| 279 | 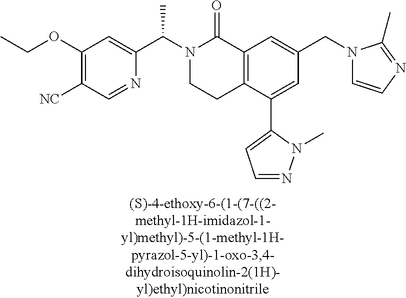 [SMILES: CCOc1cc([C@H](C)N2CCc3c(cc(Cn4ccnc4C)cc3-c3ccnn3C)C2=O)ncc1C#N] | ¹H NMR (400 MHz, CD₃OD) δ 8.61 (s, 1H), 7.89 (d, J = 2.0 Hz, 1H), 7.54 (d, J = 2.0 Hz, 1H), 7.27 (d, J = 2.0 Hz, 1H), 7.17 (s, 1H), 7.11 (d, J = 1.5 Hz, 1H), 6.88 (d, J = 1.5 Hz, 1H), 6.30 (d, J = 2.0 Hz, 1H), 5.95 (d, J = 7.2 Hz, 1H), 5.28 (s, 2H), 4.31 (q, J = 7.0 Hz, 2H), 3.70-3.44 (m, 6H), 2.75 (t, J = 6.5 Hz, 2H), 2.32 (s, 3H), 1.64 (d, J = 7.1 Hz, 3H), 1.47 (t, J = 7.0 Hz, 3H). LCMS (ESI) m/z | 6-(1-(5- bromo-7- ((2-methyl- 1H-imidazol- 1-yl)methyl)- 1-oxo-3,4- dihydroiso- quinolin- 2(1H)-yl) ethyl)-4- ethoxynico- tinonitrile (C1) | 1-methyl-5- (4,4,5,5- tetramethyl- 1,3,2- dioxaboro- lan-2-yl)- 1H-pyrazole |
| | | 496.2 [M + H]⁺. | | |
| 280 | 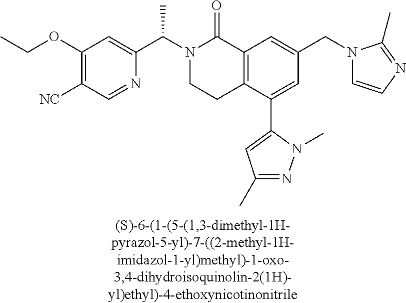 [SMILES: CCOc1cc([C@H](C)N2CCc3c(cc(Cn4ccnc4C)cc3-c3cc(C)nn3C)C2=O)ncc1C#N] | ¹H NMR (400 MHz, CD₃OD) δ 8.61 (s, 1H), 7.88 (d, J = 2.0 Hz, 1H), 7.24 (d, J = 2.0 Hz, 1H), 7.17 (s, 1H), 7.10 (d, J = 1.5 Hz, 1H), 6.88 (d, J = 1.5 Hz, 1H), 6.08 (s, 1H), 5.95 (d, J = 7.1 Hz, 1H), 5.27 (s, 2H), 4.30 (q, J = 7.0 Hz, 2H), 3.67-3.41 (m, 5H), 2.76 (t, J = 6.5 Hz, 2H), 2.32 (s, 3H), 2.25 (s, 3H), 1.64 (d, J = 7.1 Hz, 3H), 1.46 (t, J = 7.0 Hz, 3H). LCMS (ESI) m/z | 6-(1-(5- bromo-7- ((2-methyl- 1H- imidazol-1- yl)methyl)- 1-oxo-3,4- dihydroiso- quinolin- 2(1H)- yl)ethyl)-4- ethoxynico- tinonitrile (C1) | 1,3-dimethyl- 5-(4,4,5,5- tetramethyl- 1,3,2- dioxaboro- lan-2-yl)- 1H-pyrazole |
| | | 510.3 [M + H]⁺. | | |
| 281 | 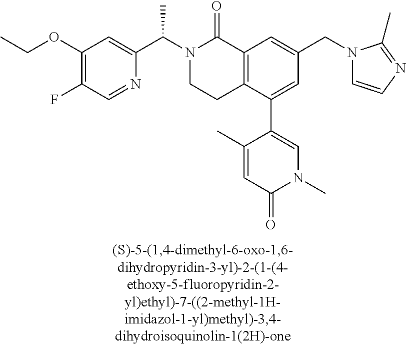 [SMILES: CCOc1cc([C@H](C)N2CCc3c(cc(Cn4ccnc4C)cc3-c3cn(C)c(=O)cc3C)C2=O)ncc1F] | ¹H NMR (400 MHz, CD₃OD) δ 8.21 (d, J = 3.3 Hz, 1H), 7.86 (t, J = 1.9 Hz, 1H), 7.45 (d, J = 16.3 Hz, 1H), 7.18-7.06 (m, 3H), 6.86 (d, J = 1.5 Hz, 1H), 6.50 (m, 1H), 5.96 (dd, J = 7.1, 2.6 Hz, 1H), 5.25 (s, 2H), 4.20 (qt, J = 7.0, 2.1 Hz, 2H), 3.60- 3.42 (m, 4H), 2.83- 2.54 (m, 2H), 2.32 (s, 3H), 1.61 (d, J = 7.1 Hz, 3H), 1.43 (td, J = 7.0, 1.1 Hz, 3H). LCMS (ESI) m/z 530.3 [M + H]⁺. | 5-bromo- 2-(1-(4- ethoxy-5- fluoro- pyridin-2- yl)ethyl)- 7-((2- methyl- 1H- imidazol- 1-yl) methyl)- 3,4- dihydroiso- quinolin- 1(2H)-one | 1,4-dimethyl- 5-(4,4,5,5- tetramethyl- 1,3,2- dioxaboro- lan-2- yl)pyridin- 2(1H)-one |
| 282 | 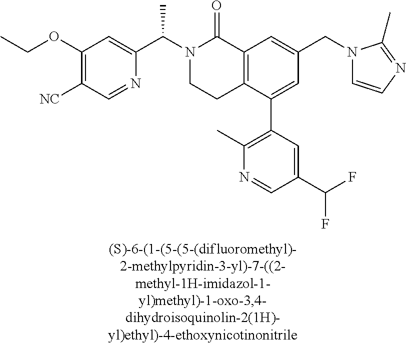 [SMILES: CCOc1cc([C@H](C)N2CCc3c(cc(Cn4ccnc4C)cc3-c3cc(C(F)F)cnc3C)C2=O)ncc1C#N] | HCO₂H: ¹H NMR (400 MHz, CD₃OD) δ 8.66 (s, 1H), 8.60 (d, J = 1.0 Hz, 1H), 8.36 (s, 1H), 7.91 (s, 1H), 7.77 (d, J = 11.2 Hz, 1H), 7.30 (dd, J = 4.8, 1.6 Hz, 2H), 7.18 (d, J = 3.6 Hz, 1H), 7.13 (d, J = 1.6 Hz, 1H), 7.08-6.76 (m, 1H), 5.98-5.92 (m, 1H), 5.37 (s, 2H), 4.34- 4.28 (m, 2H), 3.65- 3.42 (m, 2H), 2.76- 2.51 (m, 2H), 2.47 (s, 3H), 2.33 (d, J = 16.4 Hz, 3H), 1.64 (d, J = 7.0 Hz, 3H), 1.47 (t, J = 7.0 Hz, 3H). | 4-ethoxy- 6-(1-(7- ((2- methyl- 1H- imidazol- 1-yl) methyl)-1- oxo-5- (4,4,5,5- tetramethyl- 1,3,2- dioxaboro- lan-2-yl)- 3,4-dihydro- isoquinolin- 2(1H)- yl)ethyl) nicotinoni- trile (From step 1 of Example | 3-bromo- 5-(difluoro- methyl)-2- methyl- pyridine |
| | | LCMS (ESI) m/z | 264) | |
| | | 557.2 [M + H]⁺. | | |
| 283 | 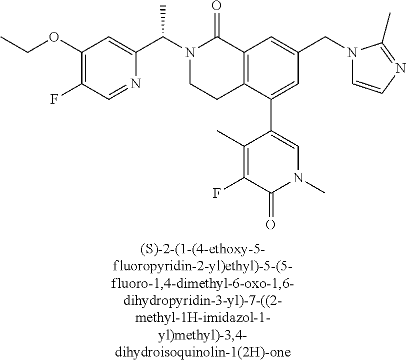 [SMILES: CCOc1cc([C@H](C)N2CCc3c(cc(Cn4ccnc4C)cc3-c3cn(C)c(=O)c(F)c3C)C2=O)ncc1F] | ¹H NMR (400 MHz, CD₃OD) δ 8.21 (m, 1H), 7.88 (s, 1H), 7.35-7.30 (m, 1H), 7.18 (s, 1H), 7.16- 7.12 (m, 1H), 7.09 (s, 1H), 6.86 (s, 1H), 5.98-5.93 (m, 1H), 5.25 (s, 2H), 4.23-4.18 (m, 2H), 3.60 (d, J = 9.2 Hz, 3H), 3.50-3.49 (m, 1H), 3.36-3.34 (m, 1H), 2.73-2.62 (m, 2H), 2.32 (s, 3H), 1.91-1.85 (m, 3H), 1.61 (d, J = 7.2 Hz, 3H), 1.44-1.41 (m, 3H). LCMS (ESI) m/z 548.3 [M + H]⁺ | 2-(1-(4- ethoxy- 5-fluoro- pyridin-2- yl)ethyl)- 7-((2- methyl- 1H- imidazol- 1-yl) methyl)- 5-(4,4,5,5- tetramethyl- 1,3,2- dioxaboro- lan-2- yl)-3,4- dihydroiso- quinolin- 1(2H)-one (Prepared from Interme- diate (C3) | 5-bromo- 3-fluoro- 1,4- dimethyl- pyridin- 2(1H)-one (G19) |
| 284 | 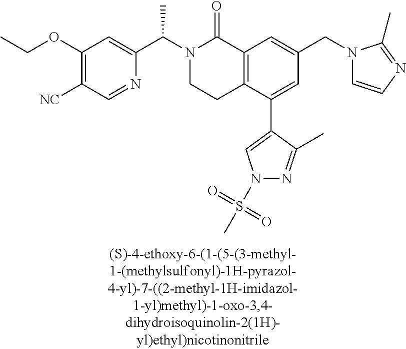 [SMILES: CCOc1cc([C@H](C)N2CCc3c(cc(Cn4ccnc4C)cc3-c3cn(S(C)(=O)=O)nc3C)C2=O)ncc1C#N] | ¹H NMR (400 MHz, DMSO-d6) δ 8.76 (s, 1H), 8.29 (s, 1H), 7.76 (s, 1H), 7.25 (d, J = 4.0 Hz, 1H), 7.21 (s, 1H), 7.18 (s, 1H), 6.77 (s, 1H), 5.91 (m, 1H), 5.23 (s, 2H), 4.32 (m, 2H), 3.55 (s, 3H), 3.44-3.30 (m, 2H), 2.72 (m, 2H), 2.33 (s, 2H), 2.24 (s, 3H), 1.56 (d, J = 8.0 Hz, 3H), 1.39 (m, 3H). LCMS (ESI) m/z 574.2 [M + H]⁺ | 6-(1-(5- bromo-7- ((2- methyl- 1H- imidazol- 1-yl) methyl)-1- oxo-3,4- dihydroiso- quinolin- 2(1H)- yl)ethyl)- 4-ethoxy- nicotino- nitrile (C1) | (3- methyl-1- (methyl- sulfonyl)- 1H- pyrazol-4- yl)boronic acid |
| 285 | 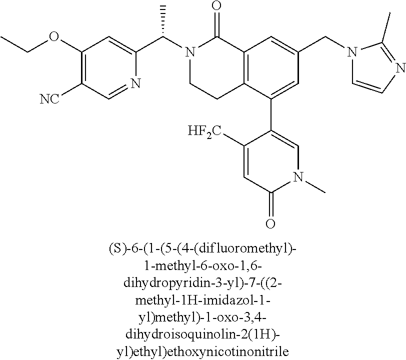 | ¹H NMR (400 MHz, Methanol-d₄) δ 8.60 (d, J = 1.8 Hz, 1H), 7.86 (d, J = 2.0 Hz, 1H), 7.63 (d, J = 16.6 Hz, 1H), 7.26- 7.18 (m, 1H), 7.17 (d, J = 6.0 Hz, 1H), 7.07 (d, J = 1.5 Hz, 1H), 6.85 (d, J = 1.5 Hz, 1H), 6.80 (d, J = 3.6 Hz, 1H), 6.36 (td, J = 54.0, 17.1 Hz, 1H), 5.95 (p, J = 7.0 Hz, 1H), 5.24 (s, 2H), 4.36-4.24 (m, 2H), 3.59 (d, J = 8.0 Hz, 3H), 3.55- 3.37 (m, 2H), 2.89- 2.56 (m, 2H), 2.30 (s, 3H), 1.64 (dd, J = 7.2, 4.7 Hz, 3H), 1.46 (td, J = 7.0, 3.7 Hz, 3H). | 4-ethoxy- 6-(1-(7- ((2-methyl- 1H- imidazol-1- yl)methyl)- 1-oxo-5- (4,4,5,5- tetramethyl- 1,3,2- dioxaboro- lan-2- yl)-3,4- dihydroiso- quinolin- 2(1H)- yl)ethyl) nicotinoni- trile (From step 1 of Example 264) | 5-bromo-4- (difluoro- methyl)-1- methyl- pyridin- 2(1H)- one (G18) |
| | | LCMS (ESI) m/z | | |
| | | 573.3 [M + H]⁺ | | |
| 286 | 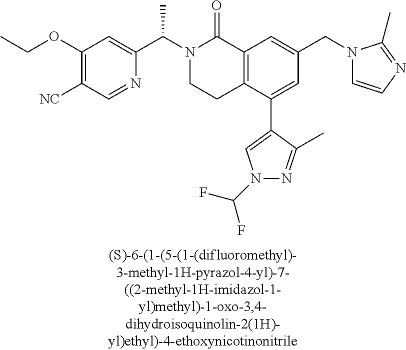 [SMILES: CCOc1cc([C@H](C)N2CCc3c(cc(Cn4ccnc4C)cc3-c3cn(C(F)F)nc3C)C2=O)ncc1C#N] | ¹H NMR (400 MHz, CD₃OD) δ 8.62 (s, 1H), 8.01 (s, 1H), 7.83 (d, J = 1.6 Hz, 1H), 7.56-7.26 (t, J = 59.6 Hz, 1H), 7.21 (d, J = 1.6 Hz, 1H), 7.18 (s, 1H), 7.12 (d, J = 1.2 Hz, 1H), 6.90 (d, J = 1.2 Hz, 1H), 5.96 (q, J = 7.2 Hz, 1H), 5.27 (s, 2H), 4.31 (q, J = 7.2 Hz, 2H), 3.64-3.42 (m, 2H), 2.83 (t, J = 6.4 Hz, 2H), 2.34 (s, 3H), 2.13 (s, 3H), 1.65 (d, J = 6.8 Hz, 3H), 1.47 (t, J = 7.0 Hz, 3H). LCMS (ESI) m/z 546.2 [M + H]⁺ | 4-ethoxy- 6-(1-(7- ((2-methyl- 1H- imidazol-1- yl)methyl)- 1-oxo-5- (4,4,5,5- tetra- methyl- 1,3,2- dioxaboro- lan-2- yl)-3,4- dihydroiso- quinolin- 2(1H)- yl)ethyl) nicotinoni- trile (From step 1 of Example 264) | 4-bromo- 1- (difluoro- methyl)-3- methyl- 1H- pyrazole |
| 287 | 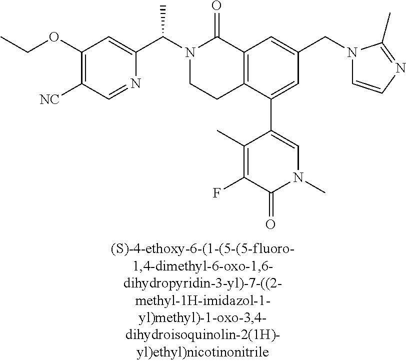 [SMILES: CCOc1cc([C@H](C)N2CCc3c(cc(Cn4ccnc4C)cc3-c3cn(C)c(=O)c(F)c3C)C2=O)ncc1C#N] | ¹H NMR (400 MHz, CD₃OD) δ 8.60 (d, J = 1.2 Hz, 1H), 7.85 (s, 1H), 7.36 (d, J = 14.0 Hz, 1H), 7.20-7.17 (m, 2H), 7.09 (s, 1H), 6.87 (s, 1H), 5.99- 5.88 (m, 1H), 5.25 (s, 2H), 4.32-4.28 (m, 2H), 3.61 (d, J = 6.8 Hz, 3H), 3.57-3.48 (m, 2H), 2.78-2.73 (m, 1H), 2.67-2.60 (m, 1H), 2.32 (s, 3H), 1.92- 1.87 (m, 3H), 1.65 (d, J = 7.2 Hz, 3H), 1.48-1.45 (m, 3H). LCMS (ESI) m/z 555.3 [M + H]⁺ | 4-ethoxy- 6-(1-(7- ((2-methyl- 1H- imidazol-1- yl)methyl)- 1-oxo-5- (4,4,5,5- tetra- methyl- 1,3,2- dioxaboro- lan-2- yl)-3,4- dihydroiso- quinolin- 2(1H)- yl)ethyl) nicotinoni- trile (From step 1 of Example 264) | 5-bromo- 3-fluoro- 1,4- dimethyl- pyridin- 2(1H)- one (G19) |
| 288 | 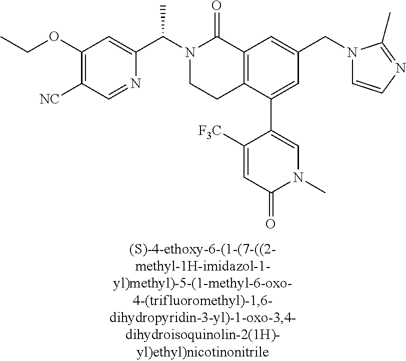 [SMILES: CCOc1cc([C@H](C)N2CCc3c(cc(Cn4ccnc4C)cc3-c3cn(C)c(=O)cc3C(F)(F)F)C2=O)ncc1C#N] | ¹H NMR (400 MHz, CDCl₃) δ 8.58 (d, J = 4.4 Hz, 1H), 8.03 (br d, J = 5.4 Hz, 1H), 7.14-7.05 (m, 1H), 7.00-6.95 (m, 3H), 6.91 (br s, 1H), 6.85 (s, 1H), 6.12- 5.98 (m, 1H), 5.19- 5.01 (m, 2H), 4.30- 4.14 (m, 2H), 3.68- 3.61 (m, 1H), 3.57 (d, J = 11.0 Hz, 3H), 3.54-3.42 (m, 1H), 3.41-3.29 (m, 1H), 2.69 (br d, J = 5.4 Hz, 1H), 2.61 (br d, J = 5.0 Hz, 2H), 2.35 (s, 3H), 1.64 (m, 3H), 1.50 (td, J = 6.9, 3.5 Hz, 3H). LCMS (ESI) m/z 591.2 [M + H]⁺. | 4-ethoxy- 6-(1-(7-((2- methyl-1H- imidazol-1- yl)methyl)- 1-oxo-5- (4,4,5,5- tetramethyl- 1,3,2- dioxaboro- lan-2- yl)-3,4- dihydroiso- quinolin- 2(1H)- yl)ethyl) nicotinoni- trile (From step 1 of Example 264) | 5-bromo- 1-methyl- 4- (trifluoro- methyl) pyridin- 2(1H)- one |
| 289 | 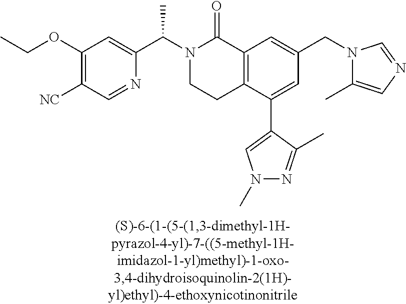 [SMILES: CCOc1cc([C@H](C)N2CCc3c(cc(Cn4cncc4C)cc3-c3cn(C)nc3C)C2=O)ncc1C#N] | ¹H NMR (400 MHz, CD₃OD) δ 8.61 (s, 1H), 7.76 (d, J = 1.9 Hz, 1H), 7.71 (d, J = 1.2 Hz, 1H), 7.52 (s, 1H), 7.17 (s, 1H), 7.11 (d, J = 2.0 Hz, 1H), 6.75 (t, J = 1.2 Hz, 1H), 5.95 (d, J = 7.1 Hz, 1H), 5.26 (s, 2H), 4.30 (q, J = 7.0 Hz, 2H), 3.84 (s, 3H), 3.61- 3.41 (m, 2H), 2.83 (td, J = 5.9, 2.9 Hz, 2H), 2.12 (d, J = 1.0 Hz, 3H), 2.06 (s, 3H), 1.64 (d, J = 7.1 Hz, 3H), 1.46 (t, J = | 6-(1-(7- (chloro- methyl)- 5-(1,3- dimethyl- 1H- pyrazol- 4-yl)-1- oxo-3,4- dihydroiso- quinolin- 2(1H)- yl)ethyl)- 4-ethoxy- nicotinoni- trile (From step 2 of Example 261) | 5-methyl- 1H- imidazole |
| | | 7.0 Hz, 3H). | | |
| | | LCMS (ESI) m/z | | |
| | | 510.1 [M + H]⁺. | | |
| 290 | 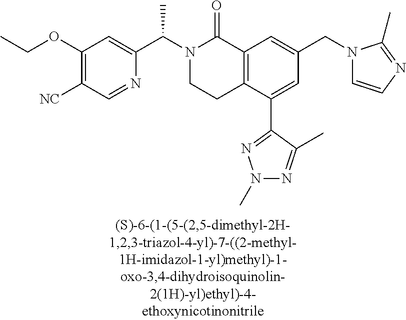 [SMILES: CCOc1cc([C@H](C)N2CCc3c(cc(Cn4ccnc4C)cc3-c3nn(C)nc3C)C2=O)ncc1C#N] | ¹H NMR (400 MHz, CD₃OD) δ 8.61 (s, 1H), 7.89 (s, 1H), 7.31 (s, 1H), 7.25- 7.13 (m, 2H), 7.04 (s, 1H), 5.96 (q, J = 7.0 Hz, 1H), 5.34 (s, 2H), 4.31 (q, J = 7.0 Hz, 2H), 4.13 (s, 3H), 3.62-3.42 (m, 2H), 2.99-2.84 (m, 2H), 2.41 (s, 3H), 2.18 (s, 3H), 1.65 (d, J = 7.0 Hz, 3H), 1.47 (t, J = 7.0 Hz, 3H). LCMS (ESI) m/z 511.3 [M + H]⁺. | 4-ethoxy- 6-(1-(7- ((2- methyl- 1H- imidazol- 1-yl) methyl)- 1-oxo-5- (4,4,5,5- tetramethyl- 1,3,2- dioxaboro- lan-2- yl)-3,4- dihydroiso- quinolin- 2(1H)- yl)ethyl) nicotinoni- (From | 4-bromo- 2,5- dimethyl- 2H-1,2,3- triazole |
| | | | step 1 of | |
| | | | Example | |
| | | | 264) | |
| 291 | 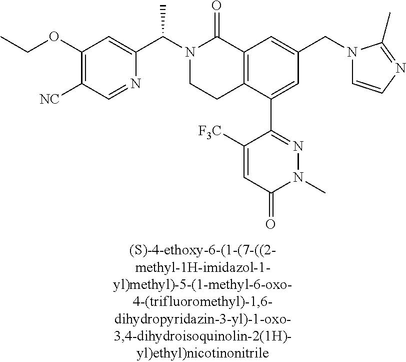 [SMILES: CCOc1cc([C@H](C)N2CCc3c(cc(Cn4ccnc4C)cc3-c3nn(C)c(=O)cc3C(F)(F)F)C2=O)ncc1C#N] | ¹H NMR (400 MHz, DMSO-d6) δ 8.75 (s, 1H), 8.02 (m, 1H), 7.75-7.50 (m, 3H), 7.46 (m, 1H), 7.23 (s, 1H), 5.88 (m, 1H), 5.49 (m, 2H), 4.32 (m, 2H), 3.71 (s, 3H), 3.50 (m, 2H), 2.82 (m, 2H), 2.70-2.50 (m, 3H), 1.55 (m, 3H), 1.38 (m, 3H). LCMS (ESI) m/z 591.8 [M + H]⁺. | 4-ethoxy- 6-(1-(7- ((2- methyl- 1H- imidazol-1- yl) methyl)-1- oxo-5- (4,4,5,5- tetramethyl- 1,3,2- dioxaboro- lan-2- yl)-3,4- dihydroiso- quinolin- 2(1H)- yl)ethyl) nicotinoni- trile (From step 1 of Example 264) | 6-bromo- 2-methyl- 5- (trifluoro- methyl) pyridazin- 3(2H)- one (G20) |
| 292 | 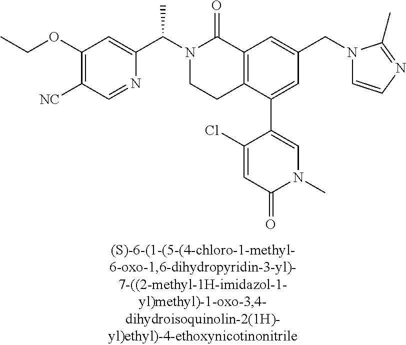 [SMILES: CCOc1cc([C@H](C)N2CCc3c(cc(Cn4ccnc4C)cc3-c3cn(C)c(=O)cc3Cl)C2=O)ncc1C#N] | LCMS (ESI) m/z 557.2 [M + H]⁺. | 1H- imidazol- 1-yl) methyl)- 1-oxo-5- (4,4,5,5- tetramethyl- 1,3,2- dioxaboro- lan-2- yl)-3,4- dihydroiso- quinolin- 2(1H)- yl)ethyl) nicotinoni- trile (From step 1 of Example 264) | 5-bromo- 4-chloro- 1-methyl- pyridin- 2(1H)- one |
| 293 | 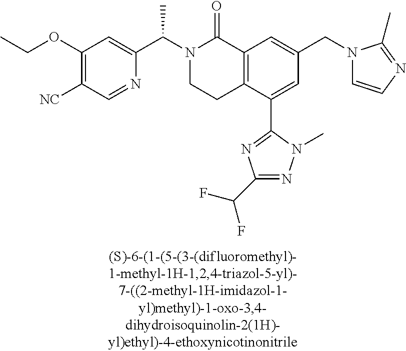 [SMILES: CCOc1cc([C@H](C)N2CCc3c(cc(Cn4ccnc4C)cc3-c3nc(C(F)F)nn3C)C2=O)ncc1C#N] | ¹H NMR (400 MHz, DMSO-d6) δ 8.75 (s, 1H), 7.89 (s, 1H), 7.58 (s, 1H), 7.30- 7.10 (m, 2H), 6.82 (m, 1H), 5.89 (m, 1H), 5.28 (m, 2H), 4.32 (m, 2H), 3.79 (s, 3H), 3.49 (m, 1H), 3.40 (m, 1H), 2.80 (m, 2H), 2.27 (m, 3H), 1.55 (m, 3H), 1.38 (m, 3H). LCMS (ESI) m/z 546.6 [M + H]⁺. | 1H- imidazol- 1-yl) methyl)- 1-oxo-5- (4,4,5,5- tetramethyl- 1,3,2- dioxaboro- lan-2- yl)-3,4- dihydroiso- quinolin- 2(1H)- yl)ethyl) nicotinoni- trile (From step 1 of Example 264) | 5-bromo- 3-(difluoro- methyl)- 1-methyl- 1H-1,2,4- triazole |
| 294 | 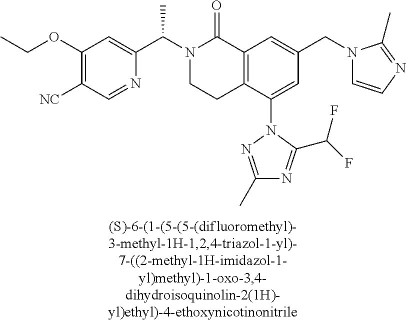 [SMILES: CCOc1cc([C@H](C)N2CCc3c(cc(Cn4ccnc4C)cc3-n3nc(C)nc3C(F)F)C2=O)ncc1C#N] | ¹H NMR (400 MHz, CD₃OD) δ 8.60 (s, 1H), 8.01 (d, J = 1.9 Hz, 1H), 7.34 (d, J = 1.9 Hz, 1H), 7.17 (s, 1H), 7.10 (d, J = 1.5 Hz, 1H), 7.04-6.71 (m, 2H), 5.94 (q, J = 7.1 Hz, 1H), 5.31 (s, 2H), 4.30 (q, J = 7.0 Hz, 2H), 3.65-3.42 (m, 2H), 2.73-2.52 (m, 2H), 2.43 (s, 3H), 2.30 (s, 3H), 1.64 (d, J = 7.1 Hz, 3H), 1.46 (t, J = 7.0 Hz, 3H). LCMS (ESI) m/z 547.2 [M + H]⁺. | 4-ethoxy- 6-(1-(5- hydrazinyl- 7-((2- methyl- 1H- imidazol-1- yl)methyl)- 1-oxo-3,4- dihydroiso- quinolin- 2(1H)- yl)ethyl) nicotinoni- trile (Following the proce- dure of step 2 of Example 265) | 2,2- difluoro- acetic acid and acetimi- damide hydro- chloride |
| 295 | 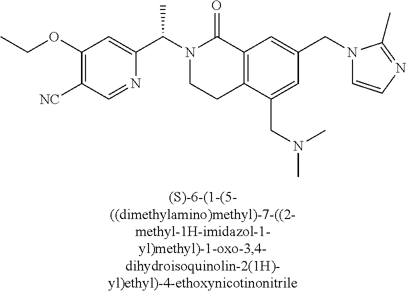 [SMILES: CCOc1cc([C@H](C)N2CCc3c(CN(C)C)cc(Cn4ccnc4C)cc3C2=O)ncc1C#N] | ¹H NMR (400 MHz, CD₃OD) δ 8.62 (s, 1H), 7.74 (d, J = 1.9 Hz, 1H), 7.25 (d, J = 1.9 Hz, 1H), 7.16 (s, 1H), 7.06 (d, J = 1.5 Hz, 1H), 6.85 (d, J = 1.4 Hz, 1H), 5.95 (d, J = 7.1 Hz, 1H), 5.20 (s, 2H), 4.35-4.22 (m, 2H), 3.68-3.48 (m, 2H), 3.45 (s, 2H), 3.10-2.87 (m, 2H), 2.30 (s, 3H), 2.20 (s, 6H), 1.65 (d, J = 7.1 Hz, 3H), 1.46 (t, J = 7.0 Hz, 3H). | 6-(1-(5- bromo-7- ((2- methyl- 1H- imidazol-1- yl)methyl)- 1-oxo-3,4- dihydroiso- quinolin- 2(1H)- yl)ethyl)- 4-ethoxy- nicotinoni- trile (C1) | potassium ((dimethyl- amino) methyl) trifluoro- borate |
| | | LCMS (ESI) m/z | | |
| | | 473.3 [M + H]⁺. | | |
| 296 | 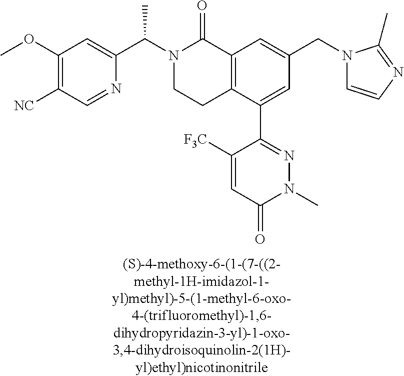 [SMILES: COc1cc([C@H](C)N2CCc3c(cc(Cn4ccnc4C)cc3-c3nn(C)c(=O)cc3C(F)(F)F)C2=O)ncc1C#N] | ¹H NMR (400 MHz, CD₃OD) δ 8.64 (s, 1H), 7.98 (s, 1H), 7.46 (s, 1H), 7.27- 7.21 (m, 2H), 7.12 (m, 1H), 6.91 (m, 1H), 5.98 (m, 1H), 5.31 (m, 2H), 4.06 (m, 3H), 3.61 (s, 3H), 3.50 (m, 2H), 2.82-2.50 (m, 2H), 2.32 (s, 3H), 1.68 (m, 3H). LCMS (ESI) m/z 578.2 [M + H]⁺. | 4-methoxy- 6-(1-(7-((2- methyl- 1H- imidazol-1- yl)methyl)- 1-oxo-5- (4,4,5,5- tetramethyl- 1,3,2- dioxaboro- lan-2- yl)-3,4- dihydroiso- quinolin- 2(1H)- yl)ethyl) nicotinoni- trile (Prepared from INT 11e) | 6-bromo- 2-methyl- 5- (trifluoro- methyl) pyridazin- 3(2H)- one (G20) |
| 297 | 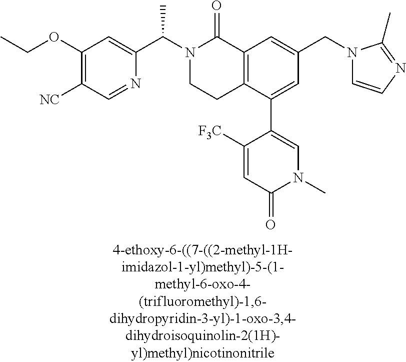 | ¹H NMR (400 MHz, DMSO-d6) δ 8.71 (s, 1H), 7.88 (s, 1H), 7.81 (s, 1H), 7.24- 7.19 (m, 3H), 6.88 (s, 2H), 5.25 (m, 2H), 4.80-4.70 (m, 2H), 4.30 (m, 2H), 3.54 (m, 2H), 3.50 (s, 3H), 2.86 (m, 1H), 2.60 (m, 1H), 2.25 (s, 3H), 1.38 (m, 3H). LCMS (ESI) m/z 577.2 [M + H]⁺. | 4-ethoxy- 6-((7-((2- methyl- 1H- imidazol- 1-yl) methyl)- 1-oxo-5- (4,4,5,5- tetramethyl- 1,3,2- dioxaboro- lan-2- yl)-3,4- dihydroiso- quinolin- 2(1H)- yl)methyl) nicotinoni- trile (Prepared from (C2) | 5-bromo- 1-methyl- 4- (trifluoro- methyl) pyridin- 2(1H)- one |
| 298 | 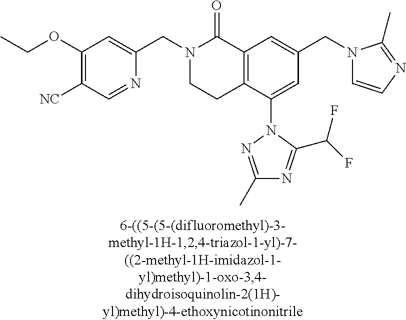 [SMILES: CCOc1cc(CN2CCc3c(cc(Cn4ccnc4C)cc3-n3nc(C)nc3C(F)F)C2=O)ncc1C#N] | ¹H NMR (400 MHz, CD₃OD) δ 8.58 (s, 1H), 8.24 (s, 1H), 8.07 (d, J = 1.9 Hz, 1H), 7.51 (d, J = 1.9 Hz, 1H), 7.37 (d, J = 1.9 Hz, 1H), 7.24 (d, J = 1.9 Hz, 1H), 7.18 (s, 1H), 7.10-6.75 (t, J = 52.1 Hz, 1H), 5.45 (s, 2H), 4.88 (s, 2H), 4.30 (q, J = 7.0 Hz, 2H), 3.69 (t, J = 6.6 Hz, 2H), 2.75 (t, J = 6.6 Hz, 2H), 2.51 (s, 3H), 2.44 (s, 3H), 1.47 (t, J = 7.0 Hz, 3H). LCMS (ESI) m/z 533.3 [M + 1]⁺. | 4-ethoxy- 6-((5- hydrazinyl- 7-((2- methyl- 1H- imidazol-1- yl)methyl)- 1-oxo-3,4- dihydroiso- quinolin- 2(1H)- yl)methyl) nicotinoni- trile (Follow- ing the procedure of step 3 of Exam- ple 265) | 2,2- difluoro- acetic acid and acetimida- mide hydro- chloride |
| 299 | 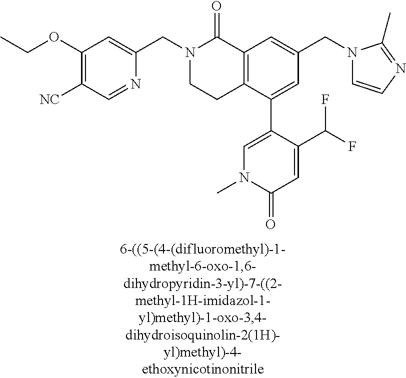 [SMILES: CCOc1cc(CN2CCc3c(cc(Cn4ccnc4C)cc3-c3cn(C)c(=O)cc3C(F)F)C2=O)ncc1C#N] | ¹H NMR (400 MHz, CD₃OD) δ 9.14-9.01 (m, 1H), 8.00 (s, 1H), 7.80-7.45 (m, 5H), 6.87 (s, 1H), 6.75- 6.25 (t, J = 52.0 Hz, 1H), 5.51 (s, 2H), 5.01 (m, 2H), 4.60 (m, 2H), 3.67 (m, 2H), 3.65 (s, 3H), 3.08 (m, 1H), 2.87 (m, 1H), 2.66 (s, 3H), 1.58 (m, 3H). LCMS (ESI) m/z 559.2 [M + H]⁺ | 4-ethoxy- 6-((7-((2- methyl- 1H- imidazol-1- yl)methyl)- 1-oxo-5- (4,4,5,5- tetramethyl- 1,3,2- dioxaboro- lan-2- yl)-3,4- dihydroiso- quinolin- 2(1H)- yl)methyl) nicotinoni- trile (Prepared from (C2)) | 5-bromo- 4- (difluoro- methyl)- 1-methyl- pyridin- 2(1H)- one (G18) |
| 300 | 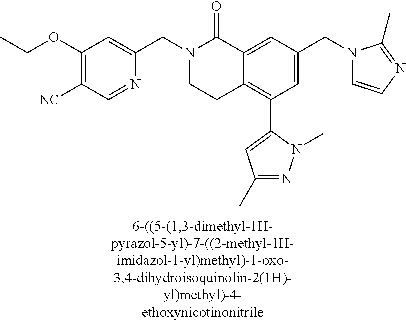 [SMILES: CCOc1cc(CN2CCc3c(cc(Cn4ccnc4C)cc3-c3cc(C)nn3C)C2=O)ncc1C#N] | ¹H NMR (400 MHz, CDCl₃) δ 8.58 (s, 1H), 8.03 (s, 1H), 7.12 (m, 2H), 7.03 (s, 1H), 6.96 (s, 1H), 5.99 (s, 1H), 5.18 (s, 2H), 4.83 (s, 2H), 4.24 (m, 2H), 3.71 (s, 2H), 3.57 (s, 3H), 2.84 (m, 2H), 2.55 (s, 3H), 2.31 (s, 3H), 1.52 (t, J = 8.0 Hz, 3H). LCMS (ESI) m/z 496.3 [M + H]⁺ | 6-((5- bromo-7- ((2- methyl- 1H- imidazol-1- yl)methyl)- 1-oxo-3,4- dihydroiso- quinolin- 2(1H)- yl)methyl)- 4-ethoxy- nicotinoni- trile (C2) | 1,3- dimethyl- 5- (4,4,5,5- tetra- methyl- 1,3,2- dioxaboro- lan-2- yl)-1H- pyrazole |
| 301 | 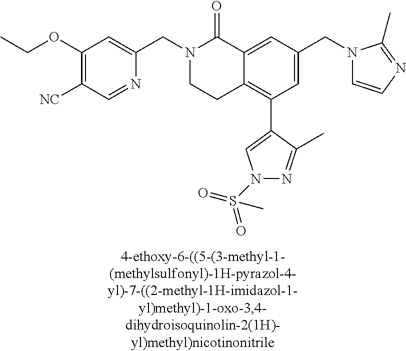 [SMILES: CCOc1cc(CN2CCc3c(cc(Cn4ccnc4C)cc3-c3cn(S(C)(=O)=O)nc3C)C2=O)ncc1C#N] | ¹H NMR (400 MHz, CDCl₃) δ 8.60 (s, 1H), 8.04 (s, 1H), 7.89 (s, 1H), 7.02 (m, 2H), 6.90 (m, 2H), 5.16 (s, 2H), 4.85 (s, 2H), 4.25 (m, 2H), 3.70 (s, 2H), 3.42 (s, 3H), 2.84 (m, 2H), 2.46 (s, 3H), 2.17 (s, 3H), 1.53 (t, J = 8.0 Hz, 3H). LCMS (ESI) m/z 560.2 [M + H]⁺ | 6-((5- bromo-7- ((2- methyl- 1H- imidazol-1- yl)methyl)- 1-oxo-3,4- dihydroiso- quinolin- 2(1H)- yl)methyl)- 4-ethoxy- nicotinoni- trile (C2) | (3-methyl- 1-(methyl- sulfonyl)- 1H- pyrazol-4- yl)boronic acid |
| 302 | 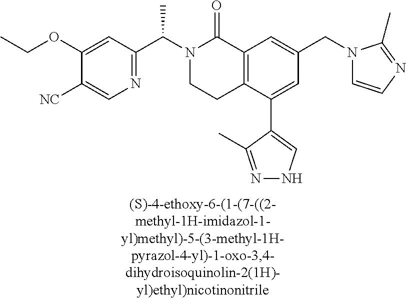 [SMILES: CCOc1cc([C@H](C)N2CCc3c(cc(Cn4ccnc4C)cc3-c3c[nH]nc3C)C2=O)ncc1C#N] | ¹H NMR (400 MHz, DMSO-d6) δ 8.76 (s, 1H), 7.78-7.60 (m, 2H), 7.44 (s, 1H), 7.23-7.07 (m, 3H), 6.77 (d, J = 1.3 Hz, 1H), 5.90 (q, J = 7.1 Hz, 1H), 5.21 (s, 2H), 4.31 (q, J = 7.0 Hz, 2H), 3.48-3.37 (m, 1H), 2.91-2.60 (m, 3H), 2.36-2.19 (m, 4H), 2.10 (d, J = 26.3 Hz, 3H), 1.54 (d, J = 7.1 Hz, 3H), 1.36 (t, J = 7.0 Hz, 3H). | 6-(1-(5- bromo- 7-((2- methyl- 1H- imidazol-1- yl)methyl)- 1-oxo-3,4- dihydroiso- quinolin- 2(1H)- yl)ethyl)- 4-ethoxy- nicotinoni- trile (C1) | 3-methyl- 4-(4,4,5,5- tetra- methyl- 1,3,2- dioxaboro- lan-2- yl)-1H- pyrazole |

### Example 303 Synthesis of (S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxypyridazine-3-carbonitrile

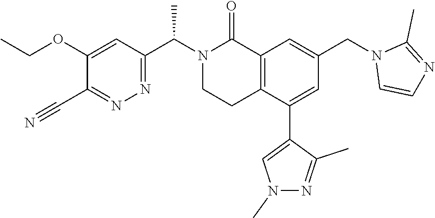 [SMILES: CCOc1cc([C@H](C)N2CCc3c(cc(Cn4ccnc4C)cc3-c3cn(C)nc3C)C2=O)nnc1C#N]

Step 1: To a solution of compound 3,4,6-trichloropyridazine (7.5 g, 41 mmol) in THF (30 mL) was added EtONa (13.94 g, 41 mmol, 1.0 eq.) in EtOH (14 mL) dropwise at 0° C. under N₂. The reaction mixture was stirred at room temperature overnight. Water (20 mL) was added to the reaction mixture and the mixture was extracted with ethyl acetate (100 mL×2). The combined organic layers were dried over anhydrous Na₂SO₄, filtered and concentrated under vacuum to give a residue, which was purified by silica column chromatography (eluting with petroleum ether:ethyl acetate=10:1-5:1) to give 3,6-dichloro-4-ethoxypyridazine as a yellow solid. ¹H NMR (400 MHz, CDCl₃) δ 6.90 (s, 1H), 4.21 (q, J=7.0 Hz, 2H), 1.56 (t, J=7.0 Hz, 3H). LCMS: [M+H]⁺=193.0.

Step 2: A mixture of 3,6-dichloro-4-ethoxypyridazine (from step 1, 3 g, 15.5 mmol), tributyl(1-ethoxyvinyl)stannane (6.7 g, 18.6 mmol, 1.2 eq.), Pd(PPh₃)₄ (900 mg, 0.775 mmol, 0.05 eq.) in toluene (25 mL) was stirred at 110° C. for 16 hours under N₂. The mixture was concentrated under vacuum and was purified by flash chromatography on silica gel (petroleum ether:ethyl acetate=5:1) to give 3-chloro-4-ethoxy-6-(1-ethoxyvinyl)pyridazine as a pale-yellow solid. LCMS: [M+H]⁺=228.9.

Step 3: A mixture of 3-chloro-4-ethoxy-6-(1-ethoxyvinyl)pyridazine (From step 2, 1.96 g, 8.56 mmol), Pd₂(dba)₃ (754 mg, 0.86 mmol, 0.1 eq.), dppf) (950 mg, 1.71 mmol, 0.2 eq.) and Zn(CN)₂ (601 mg, 5.14 mmol, 0.6 eq.) in DMF (25 mL) was stirred at 110° C. under N₂ for 16 hours. Solvent was removed under reduced pressure and the residue was purified by flash chromatography on silica gel (MeOH/DCM=0-10%) to give 4-ethoxy-6-(1-ethoxyvinyl)pyridazine-3-carbonitrile as a brown solid. LCMS: [M+H]⁺=220.2.

Step 4: To a solution of 4-ethoxy-6-(1-ethoxyvinyl)pyridazine-3-carbonitrile (From step 3, 800 mg, 3.65 mmol) in THF (10 mL) at 25° C. was added aqueous HCl (3 N, 5 mL, 15.0 mmol) and the reaction mixture was stirred at 25° C. for 2 hours. The resulting mixture was concentrated in vacuo, extracted with EtOAc, dried over anhydrous Na₂SO₄ and then filtered. The filtrate was concentrated to afford 6-acetyl-4-ethoxypyridazine-3-carbonitrile as a brown oil, which was used in next step directly. LCMS: [M+H]⁺=192.1.

Step 5: To a solution of 6-acetyl-4-ethoxypyridazine-3-carbonitrile (From step 4, 602 mg, 3.2 mmol) in EtOH (20 mL) at 0° C. was added NaBH₄ (179 mg, 4.7 mmol, 1.5 eq.), and the reaction mixture was then stirred at 25° C. for 1 hour. Saturated aqueous NH₄Cl (5 mL) was added to the mixture and the mixture was concentrated in vacuo. To the residue was added EtOAc (10 mL), washed with water and brine. The organic phase was dried over anhydrous Na₂SO₄ and then filtered. The filtrate was concentrated to afford 4-ethoxy-6-(1-hydroxyethyl)pyridazine-3-carbonitrile as a brown solid. ¹H NMR (400 MHz, CDCl₃) δ 7.24 (s, 1H), 5.22 (q, J=6.6 Hz, 1H), 4.31 (q, J=7.0 Hz, 1H), 1.62 (d, J=6.6 Hz, 1H), 1.56 (t, J=7.0 Hz, 3H). LCMS: [M+H]⁺=194.1.

Step 6: To a solution of 4-ethoxy-6-(1-hydroxyethyl)pyridazine-3-carbonitrile (From step 5, 212 mg, 1.1 mmol) and PPh₃ (339 mg, 1.3 mmol, 1.2 eq.) in DCM (20 mL) was added a solution of CBr₄ (445 mg, 1.36 mmol, 1.2 eq.) in DCM (20 mL) at 0-10° C. The mixture was then stirred at 20° C. for 1 hour. The resulting mixture was concentrated to dryness, and the residue was purified by flash chromatography on silica gel (PE/EA=10:1 to 2:1) to give 6-(1-bromoethyl)-4-ethoxypyridazine-3-carbonitrile as a brown solid. ¹H NMR (400 MHz, CDCl₃) δ 7.23 (s, 1H), 5.44 (q, J=7.0 Hz, 1H), 4.33 (q, J=7.0 Hz, 1H), 2.12 (d, J=7.0 Hz, 3H), 1.58 (t, J=7.0 Hz, 3H). LCMS: [M+H]⁺=255.9.

Step 7: To a mixture of 5-(1,3-dimethyl-1H-pyrazol-4-yl)-7-((2-methyl-1H-imidazol-1-yl)methyl)-3,4-dihydroisoquinolin-1(2H)-one (163 mg, 0.49 mmol, 0.9 eq.) and DMF (3 mL) was added NaH (60%, 30 mg, 0.75 mmol, 1.39 eq.) at 0° C. The mixture was stirred for 30 minutes and then 6-(1-bromoethyl)-4-ethoxypyridazine-3-carbonitrile (From step 6, 138 mg, 0.54 mmol, 1.0 eq.) was added. The reaction mixture was stirred at 0° C. for 1 hour. Water was added to the reaction mixture and the mixture was extracted with EtOAc (30 mL×3). The combined organic layers were dried over anhydrous Na₂SO₄ and then filtered. The filtrate was concentrated and purified by flash chromatography on silica gel (MeOH/DCM=0-10%) to give the residue, which was further purified by prep-HPLC (basic mobile phase) to give the racemic product as an off-white solid. The racemic product was separated by chiral prep-HPLC (Column: Xbridge C18 150*19 mm, 5 um; mobile phase: water (0.05% ammonia hydroxide)-ACN; ACN %: 20%-50%) to afford (S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxypyridazine-3-carbonitrile as a white solid (Peak 2 on the chiral column). ¹H NMR (400 MHz, DMSO-d6) δ 7.88 (d, J=1.7 Hz, 1H), 7.19 (d, J=8.7 Hz, 2H), 7.02 (dd, J=7.7, 3.8 Hz, 2H), 6.91 (s, 1H), 6.14 (q, J=7.2 Hz, 1H), 5.12 (s, 2H), 4.29 (q, J=7.0 Hz, 2H), 3.89 (s, 3H), 3.72-3.52 (m, 2H), 2.99-2.67 (m, 2H), 2.45 (s, 3H), 2.10 (s, 3H), 1.80 (d, J=7.2 Hz, 3H), 1.56 (t, J=7.0 Hz, 3H). LCMS: [M+H]⁺=511.1.

### Example 304: Synthesis of 4-ethoxy-6-(1-(7-(2-(ethyl(methyl)amino)ethyl)-1-oxo-5-(1,1,1-trifluoro-2-hydroxypropan-2-yl)-3,4-dihydroisoquinolin-2(1H)-yl)ethyl)nicotinonitrile (304)

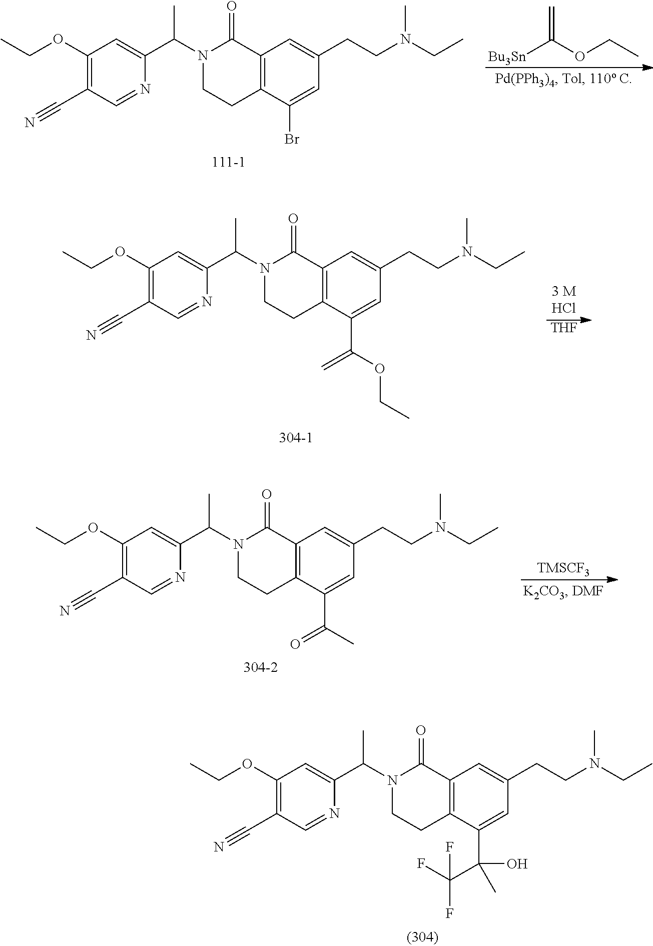

Step 1: A mixture of compound 111-1 (200 mg, 0.41 mmol, 1.0 eq), ethyl tributylstannanecarboxylate (180 mg, 0.50 mmol, 1.2 eq) and Pd(PPh₃)₄ (47 mg, 0.04 mmol, 0.1 eq) in Tol (10 mL) was stirred at 110° C. for 16 hrs under N₂. LCMS showed compound 111-1 was consumed. The mixture was concentrated under vacuum and purified by silica gel Flash Chromatography (CH₂Cl₂:MeOH=0-10%) to give 4-ethoxy-6-(1-(5-(1-ethoxyvinyl)-7-(2-(ethyl(methyl)amino)ethyl)-1-oxo-3,4-dihydroisoquinolin-2(1H)-yl)ethyl)nicotinonitrile (304-1) as a white oil. LC-MS: 477.3 [M+H]⁺.

Step 2: A mixture of compound 304-1 (160 mg, 0.34 mmol, 1.0 eq) in 3 M HCl (1 mL) and THF (5 mL) was stirred at rt for 2 hrs. LCMS showed compound 304-1 was consumed. The mixture was basified with sat. NaHCO₃ (20 mL) and extracted with acetate ethyl (40 mL×2). The combined organic layers were washed with brine (20 mL), dried over Na₂SO₄ and concentrated under vacuum to give 6-(1-(5-acetyl-7-(2-(ethyl(methyl)amino)ethyl)-1-oxo-3,4-dihydroisoquinolin-2(1H)-yl)ethyl)-4-ethoxynicotinonitrile (304-2) as a white solid. The crude product was used for the next step directly. LC-MS: 449.0 [M+H]⁺.

Step 3: To a solution of compound 304-2 (150 mg, 0.33 mmol, 1.0 eq), K₂CO₃ (5 mg, 0.03 mmol, 0.1 eq) in DMF (7 mL) was added TMSCF₃ (95 mg, 0.66 mmol, 2.0 eq). The mixture was stirred at rt for 16 hrs. The reaction mixture was diluted with acetate ethyl (60 mL), washed with brine (15 mL×2) and concentrated under vacuum and purified by silica gel Flash Chromatography (CH₂Cl₂:MeOH=0-10%) to give 4-ethoxy-6-(1-(7-(2-(ethyl (methyl) amino) ethyl)-1-oxo-5-(1,1,1-trifluoro-2-hydroxypropan-2-yl)-3,4-dihydroisoquinolin-2(1H)-yl)ethyl) nicotinonitrile (304) as a white solid. ¹H NMR (400 MHz, Methanol-d₄) δ 8.63 (d, J=6.5 Hz, 1H), 7.91 (s, 1H), 7.55 (d, J=6.8 Hz, 1H), 7.18 (d, J=3.5 Hz, 1H), 5.94 (m, 1H), 4.31 (q, J=7.0 Hz, 2H), 3.65-3.15 (m, 6H), 3.01-2.70 (m, 6H), 1.84 (s, 3H), 1.66 (t, J=7.0 Hz, 3H), 1.47 (t, J=7.0 Hz, 3H), 1.17 (t, J=7.2 Hz, 3H). LC-MS: 519.3 [M+H]⁺.

### Example 305: Synthesis of 4-ethoxy-6-((7-(2-(ethyl(methyl)amino)ethyl)-1-oxo-5-(2,2,2-trifluoro-1-methoxyethyl)-3,4-dihydroisoquinolin-2(1H)-yl)methyl)nicotinonitrile (305)

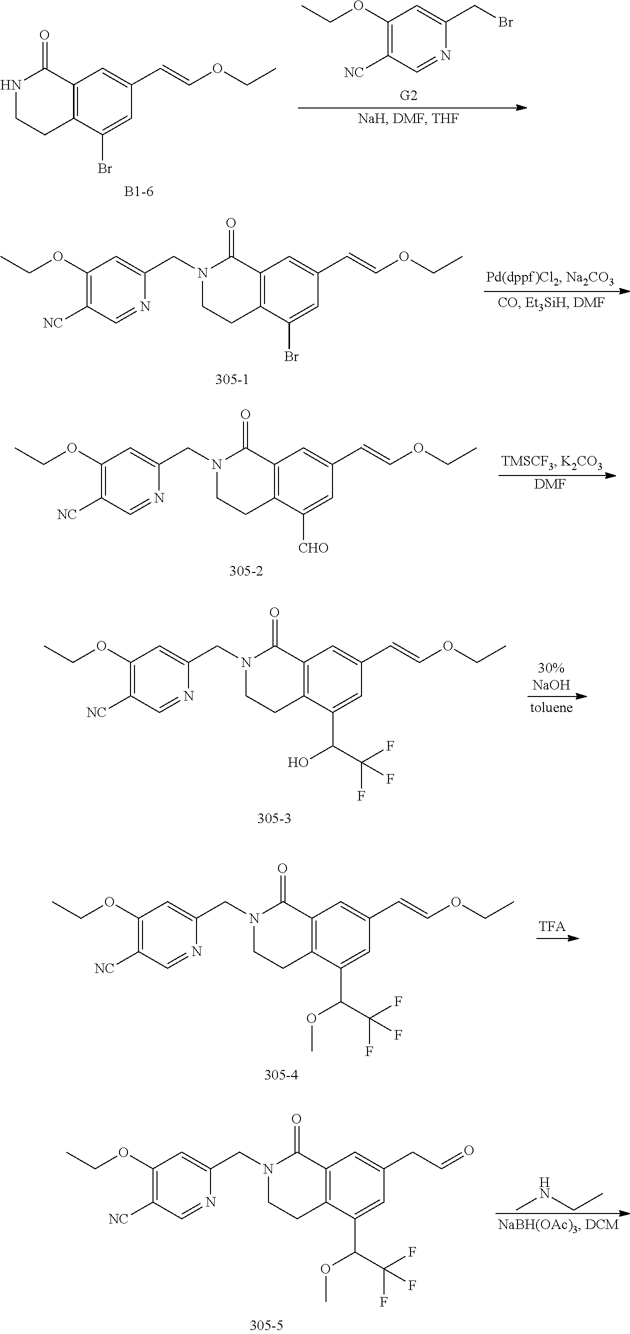

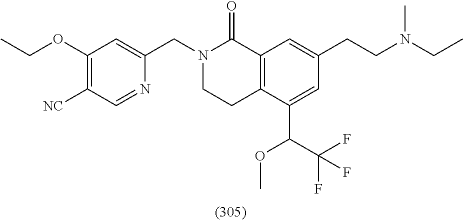 [SMILES: CCOc1cc(CN2CCc3c(cc(CCN(C)CC)cc3C(OC)C(F)(F)F)C2=O)ncc1C#N]

Step 1: (E)-6-((5-bromo-7-(2-ethoxyvinyl)-1-oxo-3,4-dihydroisoquinolin-2(1H)-yl)methyl)-4-ethoxynicotinonitrile (305-1) was obtained using intermediates (B1-6) and (G2), and the method described in step 1 of Example 111.

Step 2: A mixture of compound 305-1 (1.47 g, 3.2 mmol, 1.0 eq), Na₂CO₃ (343 mg, 3.2 mmol, 1 eq), Pd(dppf)Cl₂.DCM (37 mg, 0.16 mmol, 0.05 eq) and TES (742 mg, 6.4 mmol, 2 eq) in DMF (20 mL) was stirred at 90° C. for 16 hrs under CO atmosphere (balloon). The mixture was diluted with water (50 mL) and extracted with acetate ethyl (30 mL×3). The combined organic layers were washed with brine (50 mL), dried over Na₂SO₄ and concentrated under vacuum and purified by silica gel Flash Chromatography (MeOH:CH₂Cl₂=0-10%) to give (E)-4-ethoxy-6-((7-(2-ethoxyvinyl)-5-formyl-1-oxo-3,4-dihydroisoquinolin-2(1H)-yl)methyl)nicotinonitrile (305-2) as a yellow oil. LC-MS: 405.8 [M+H]⁺.

Step 3: A mixture of compound 305-2 (350 mg, 0.86 mmol, 1.0 eq), TMSCF₃ (245 mg, 1.72 mmol, 2.0 eq), K₂CO₃ (12 mg, 0.03 mmol, 0.1 eq) in DMF (15 mL) was stirred at rt for 16 h. The reaction mixture was diluted with water (50 mL) and extracted with acetate ethyl (30 mL×3). The combined organic layers were concentrated under vacuum. The residue was treated with 1 N HCl solution (10 mL) and stirred at rt for 1 h. The mixture was extracted with acetate ethyl (20 mL×3). The combined organic layers were washed with sat. NaHCO₃ solution (20 mL×2), dried over Na₂SO₄, concentrated under vacuum and purified by silica gel Flash Chromatography (MeOH/CH₂Cl₂=0-5%) to give (E)-4-ethoxy-6-((7-(2-ethoxyvinyl)-1-oxo-5-(2,2,2-trifluoro-1-hydroxyethyl)-3,4-dihydroisoquinolin-2(1H)-yl)methyl)nicotinonitrile (305-3) as a yellow oil. LC-MS: 475.8 [M+H]⁺.

Step 4: To a solution of compound 305-3 (240 mg, 0.5 mmol, 1.0 eq) in toluene (15 mL) was added 30% aqueous NaOH solution (7 mL), TBAB (80 mg, 0.25 mmol, 0.5 eq) and iodomethane (142 mg, 1 mmol, 2 eq) at 0° C. The resulting mixture was stirred at 30° C. overnight. The reaction mixture was diluted with water (20 mL) and extracted with acetate ethyl (3×20 mL). The combined organic phase was washed with brine (20 mL), dried over Na₂SO₄, filtered and concentrated to get a residue. The residue was purified by silica gel flash column chromatography (DCM/MeOH=0-3%) to give (E)-4-ethoxy-6-((7-(2-ethoxyvinyl)-1-oxo-5-(2,2,2-trifluoro-1-methoxyethyl)-3,4-dihydroisoquinolin-2(1H)-yl)methyl)nicotinonitrile (305-4) as a brown oil. LC-MS: 489.8 [M+H]⁺.

Step 5: To a solution of compound 305-4 (160 mg, 0.347 mmol, 1.0 eq) in DCM (6 mL) was added TFA (2 mL) at 0° C. The mixture was stirred at rt for 1 hours. The reaction mixture was basified by aq. NaHCO₃ to pH=8 and extracted with dichloromethane (20 mL×3). The combined organic layers were washed with brine (20 mL), dried over Na₂SO₄ and filtered to afford 4-ethoxy-6-((1-oxo-7-(2-oxoethyl)-5-(2,2,2-trifluoro-1-methoxyethyl)-3,4-dihydroisoquinolin-2(1H)-yl)methyl)nicotinonitrile (304-5) as brown solid (150 mg crude), which was used for the next step. LC-MS: 462.2 [M+H]⁺.

Step 6: To a mixture of compound 305-5 (150 mg, 0.32 mmol, 1 equiv) in dichloromethane (20 mL) was added N-methylethanamine (56.7 mg, 0.96 mmol, 3 equiv) at 25° C. The reaction mixture was stirred at rt for 20 min. Then NaBH(OAc)₃ (203 mg, 0.96 mmol, 3 equiv) was added portions at rt. The reaction mixture was stirred at 20° C. for 16 hours. The mixture was acidified with aq. HCl (2 M) to pH=3 and diluted with water. The organic layer was abandoned. The aqueous layer was adjusted to pH=8 with aq. Na₂CO₃. Then the aqueous layer was extracted with ethyl acetate (20 mL×3). The organic layers were washed with brine, dried over Na₂SO₄, filtered and concentrated. The residue was purified by silica gel Flash Chromatography (CH₂Cl₂:MeOH=0-10%) to give 4-ethoxy-6-((7-(2-(ethyl(methyl)amino)ethyl)-1-oxo-5-(2,2,2-trifluoro-1-methoxyethyl)-3,4-dihydroisoquinolin-2(1H)-yl)methyl)nicotinonitrile (305) as a white solid. ¹H NMR (400 MHz, CDCl₃) δ 8.58 (s, 1H), 8.03 (d, J=1.8 Hz, 1H), 7.53 (s, 1H), 7.02 (s, 1H), 4.92-4.72 (m, 3H), 4.22 (q, J=7.0 Hz, 2H), 3.76-3.62 (m, 2H), 3.44 (s, 3H), 3.03 (t, J=6.4 Hz, 4H), 2.94-2.70 (m, 4H), 2.52 (s, 3H), 1.49 (t, J=7.0 Hz, 3H), 1.24 (t, J=5.4 Hz, 3H). LC-MS: 504.8 [M+H]⁺.

### Biological Activity

Inhibition of WDR5 was measured using the following methods.

### WDR5 Assay:

### WDR5 HTRF Binding Assay

The compounds were serially diluted 3-fold in DMSO to obtain a total of twelve concentrations. Then the test compounds at each concentration (90 nL of each) were transferred by Mosquito in the white Proxiplate plus 384-well microplate (Perkin Elmer). The assay mixture (typical 9 μL) containing 6 nM WDR5 and 20 nM Biotin-MLL1 WIN peptide (3762-3773) in the assay buffer (25 mM Hepes, pH7.5, 0.05% Tween 20, 2 mM DTT, 100 mM NaCl, 0.1% BSA) were added to the wells. For counter assay, a 9 μL solution of 15 nM Biotin-PEG-His peptide was added into the wells. After 20 min incubation at rt, a 3 μL detection solution containing 14 μg/ml Phycolink Streptavidin Allophycocyanin (Prozyme), 2 nM Anti-His-Eu (PerkinElmer) in assay buffer was then added. After 1 hr incubation, the plate was read in the EnVision reader (PerkinElmer) using the HTRF protocol (The protocol set-up: Lance/Delfia dual enhancer mirror, excitation at 320 UV2 (TRF), emission at APC665, 2^{nd} emission at EuropiμM 615). HTRF signals were normalized based on the reading coming from the positive (maximum signal control) and negative controls (minimum signal control) to give percentage of activities left. The data were then fit to a dose response equation using the program Helios (Novartis) to get the IC₅₀ values. Helios is a Novartis in-house assay data analysis software using the methods described by Normolle, D. P., Statistics in Medicine, 12:2025-2042 (1993); Formenko, I. et al., Computer Methods and programs in Biomedicine, 82, 31-37 (2006); Sebaugh, J. L., Pharmaceutical Statistics, 10:128-134 (2011); Kelly, C. et al., Biometrics, 46(4):1071-1085 (1990); and Kahm, M, et al., Journal of Statistical Software, 33(7):(2010) (grofit: Fitting Biological Growth Curves with R, pages 1-21, available at http://www.jstatsoft.org/).

### MLL1 LC-MS Assay

The compounds of the present disclosure were serially and separately diluted 3-fold in DMSO to obtain a total of eight or twelve concentrations. Then the test compounds at each concentration (120 nL of each) were transferred by Mosquito into white Proxiplate plus 384-well microplate (PerkinElmer). Solutions (6 μL) of 60 nM wild type MLL1 four-member complex (MLL1-4C) and 5 μM SAM in the reaction buffer (20 mM Tris-HCl, pH8.0, 0.01% Tween 20, 1 mM DTT, 10 mM MgCl₂, 0.01% BSA) were added to the wells that were then incubated with the test compound for 20 min. A 6 μL solution of 20 μM of the peptide substrate H3K4me0 (histone H3[1-21]-Biotin) in the reaction buffer was added to initiate each reaction. The final components in the reaction solution include 30 nM MLL1-4C, 2.5 μM SAM, and 10 μM H3K4me0 with varying concentration of the compounds. A positive control consisted of 30 nM MLL1-4C, 2.5 μM SAM, and 10 μM substrate in the absence of the test compound, and a negative control consisted of 2.5 μM SAM, and 10 μM substrate only. Each reaction was incubated at rt for 120 min, then stopped by addition of 3 μL per of quench solution (2.5% TFA with 320 nM d4-SAH). The reaction mixture was centrifuged (Eppendorf centrifuge 5810, Rotor A-4-62) for 2 min at 2000 rmp. The SAH production from the enzymatic assays were monitored by LC-MS/MS on an API 4000 triple quadrupole mass spec with Turbolon Spray (Applied Biosystem) coupled with Prominenece UFLC (Shimazu). The level of SAH production were then normalized based on the values coming from the positive and negative controls to give percent enzyme activities. The data were then fit to a dose response equation using the program Helios (Novartis) to get the IC₅₀ values of the test compound. Helios is a Novartis in-house assay data analysis software using the methods described by Normolle, D. P., Statistics in Medicine, 12:2025-2042 (1993); Formenko, I. et al., Computer Methods and programs in Biomedicine, 82, 31-37 (2006); Sebaugh, J. L., Pharmaceutical Statistics, 10:128-134 (2011); Kelly, C. et al., Biometrics, 46(4):1071-1085 (1990); and Kahm, M, et al., Journal of Statistical Software, 33(7):(2010) (grofit: Fitting Biological Growth Curves with R, pages 1-21, available at http://www.jstatsoft.org/).

### 293T WDR5-MLL NanoBit Assay

SmBiT-WDR5 and MLL1(3745-3969aa)-LgBiT (20 ng:20 ng per well) were mixed with 3-fold Fugene HD (m/v, Promega, #E2311) in Opti-MEM (1 μl/well, Gibco #11058-021). 10 min after incubation at room temperature, the transfection mixture was mixed with 293T cells and seeded into 384-well plates (Greiner, #781080) with a final volume of 40 ul and a concentration of 10,000 cells per well. 48 h after transfection, 200 nl of cmpd dilution series were added to yield final drug concentration ranges of 50 μM-0.28 nM (12-point dose response assays) by 3-fold dilutions and a final DMSO concentration of 0.5%. 24 h after cmpd adding, 10 ul of Nano-Glo working solution (Promega, #N2013) was added and incubated at 37° C. for 1.5 h. Luminescence per well was then measured using plate reader (Perkin Elmer, Enspire). Dose-response curves were generated by Prism, and the half-maximal inhibitory concentration (IC₅₀) or EC₅₀ was calculated, which reflects the inhibition of WDR5-MLL1 protein-protein interaction in cells.

### MV4-11 6-Day Cell Growth CTG (CellTiter-Glo) Assay

Acute myeloid leukemia cell MV4-11 (ATCC® CRL-9591™) was cultured with RPMI 1640 medium (Thermo Fisher Scientific, cat #11875) supplemented with 10% FBS (Thermo Fisher Scientific, cat #10099141) in humidified incubator at 37° C., 5% CO₂. To assess the effect of WDR5 inhibitor on cell growth, the compound of the present invention was dissolved in DMSO and serially diluted at 1:3 for 12 points starting from 10 mM then 200 nL for the replicate of each dose per well was dispensed to Viewplate-384 Black (Perkin Elmer). Exponentially growing MV4-11 cells were seeded at the density of 300 cells per well in 40 μL to the plate, so the final compound working concentration starts from 50 μM. After 6 days, 40 μL CellTiter-Glo (Promega, cat #G7573) was added into the cell culture well and luminescence was read with Envision (Perkin Elmer) to determine the viable cells. The percentage inhibition was calculated against the samples treated with DMSO only and the data were used for dose response curve fitting in GraphPad Prism to get the IC₅₀ of representative compound of present invention, which reflects the inhibition of WDR5 activity in the proliferation of MV4;11.

Table 1 lists (a) WDR5 HTRF Pep Val v.1[Inhibitor/IC50]Qualified AC50 (μM), (b) MLL LCMS Peptide Val.v.1 Qualified AC50 (μM), (c) MLL/WDR5 cell proliferation assay//MLL/WDR5 proliferation MV4:11 Qualified AC50 (μM) measured for the following examples.

TABLE 1

| Ex # | (a) | (b) | (c) |
|---|---|---|---|
| 1 | ++ | ++ | ++ |
| 2 | ++ | ++ | ++ |
| 3 | +++ | ++ | ++ |
| 4 | ++ | ++ | ++ |
| 5 | ++ | ++ | ++ |
| 6 | ++ | ++ | ++ |
| 7 | +++ | ++ | ++ |
| 8 | +++ | ++ | ++ |
| 9 | +++ | ++ | ++ |
| 10 | +++ | ++ | ++ |
| 11 | +++ | ++ | ++ |
| 12 | ++ | ++ | ++ |
| 13 | ++ | ++ | ++ |
| 14 | ++ | ++ | ++ |
| 15 | ++ | ++ | ++ |
| 16 | ++ | ++ | ++ |
| 17 | ++ | ++ | ++ |
| 18 | ++ | ++ | ++ |
| 19 | ++ | ++ | ++ |
| 20 | ++ | ++ | ++ |
| 21 | ++ | ++ | ++ |
| 22 | ++ | ++ | ++ |
| 23 | ++ | ++ | ++ |
| 24 | ++ | ++ | ++ |
| 25 | ++ | ++ | ++ |
| 26 | ++ | ++ | ++ |
| 27 | ++ | ++ | ++ |
| 28 | ++ | ++ | ++ |
| 29 | ++ | ++ | ++ |
| 30 | ++ | ++ | ++ |
| 31 | ++ | ++ | ++ |
| 32 | ++ | ++ | ++ |
| 33 | ++ | ++ | ++ |
| 34 | ++ | ++ | ++ |
| 35 | ++ | ++ | ++ |
| 36 | ++ | ++ | ++ |
| 37 | ++ | ++ | ++ |
| 38 | ++ | ++ | ++ |
| 39 | ++ | ++ | ++ |
| 40 | ++ | ++ | ++ |
| 41 | ++ | ++ | ++ |
| 42 | ++ | ++ | ++ |
| 43 | ++ | ++ | ++ |
| 44 | ++ | ++ | ++ |
| 45 | ++ | ++ | ++ |
| 46 | ++ | ++ | ++ |
| 47 | ++ | ++ | ++ |
| 48 | ++ | ++ | ++ |
| 49 | ++ | ++ | ++ |
| 50 | ++ | ++ | ++ |
| 51 | +++ | ++ | ++ |
| 52 | +++ | ++ | ++ |
| 53 | ++ | ++ | ++ |
| 54 | ++ | ++ | ++ |
| 55 | ++ | ++ | ++ |
| 56 | ++ | ++ | ++ |
| 57 | ++ | ++ | ++ |
| 58 | ++ | ++ | ++ |
| 59 | ++ | ++ | ++ |
| 60 | ++ | ++ | ++ |
| 61 | ++ | ++ | ++ |
| 62 | ++ | ++ | ++ |
| 63 | ++ | ++ | ++ |
| 64 | ++ | ++ | ++ |
| 65 | ++ | ++ | ++ |
| 66 | ++ | ++ | ++ |
| 67 | ++ | ++ | ++ |
| 68 | ++ | ++ | ++ |
| 69 | ++ | ++ | ++ |
| 70 | ++ | ++ | ++ |
| 71 | ++ | ++ | ++ |
| 72 | ++ | ++ | ++ |
| 73 | ++ | ++ | ++ |
| 74 | ++ | ++ | ++ |
| 75 | +++ | ++ | ++ |
| 76 | +++ | ++ | ++ |
| 77 | ++ | ++ | ++ |
| 78 | +++ | ++ | ++ |
| 79 | ++ | ++ | ++ |
| 80 | ++ | ++ | ++ |
| 81 | ++ | ++ | ++ |
| 82 | ++ | ++ | ++ |
| 83 | ++ | ++ | ++ |
| 84 | ++ | ++ | ++ |
| 85 | ++ | ++ | ++ |
| 86 | ++ | ++ | ++ |
| 87 | ++ | ++ | ++ |
| 88 | +++ | ++ | ++ |
| 89 | +++ | ++ | ++ |
| 90 | ++ | ++ | ++ |
| 91 | ++ | ++ | ++ |
| 92 | ++ | ++ | ++ |
| 93 | ++ | ++ | ++ |
| 94 | ++ | ++ | ++ |
| 95 | ++ | ++ | ++ |
| 96 | ++ | ++ | ++ |
| 97 | ++ | ++ | ++ |
| 98 | +++ | ++ | ++ |
| 99 | ++ | ++ | ++ |
| 100 | ++ | ++ | ++ |
| 101 | ++ | ++ | ++ |
| 102 | ++ | ++ | ++ |
| 103 | ++ | ++ | ++ |
| 104 | ++ | ++ | ++ |
| 105 | ++ | ++ | ++ |
| 106 | ++ | ++ | ++ |
| 107 | ++ | ++ | ++ |
| 108 | ++ | ++ | ++ |
| 109 | ++ | ++ | n.d. |
| 110 | ++ | ++ | n.d. |
| 111 | +++ | ++ | ++ |
| 112 | +++ | ++ | ++ |
| 113 | +++ | ++ | ++ |
| 114 | +++ | ++ | ++ |
| 115 | +++ | ++ | ++ |
| 116 | +++ | ++ | ++ |
| 117 | +++ | ++ | ++ |
| 118 | +++ | ++ | ++ |
| 119 | +++ | ++ | ++ |
| 120 | +++ | ++ | ++ |
| 121 | +++ | ++ | ++ |
| 122 | +++ | ++ | ++ |
| 123 | +++ | ++ | ++ |
| 124 | +++ | ++ | ++ |
| 125 | +++ | ++ | ++ |
| 126 | +++ | ++ | ++ |
| 127 | +++ | ++ | ++ |
| 128 | +++ | ++ | ++ |
| 129 | +++ | ++ | ++ |
| 130 | +++ | ++ | ++ |
| 131 | +++ | ++ | ++ |
| 132 | +++ | ++ | ++ |
| 133 | +++ | ++ | ++ |
| 134 | +++ | ++ | ++ |
| 135 | +++ | ++ | ++ |
| 136 | +++ | ++ | ++ |
| 137 | +++ | ++ | ++ |
| 138 | +++ | ++ | ++ |
| 139 | +++ | ++ | ++ |
| 140 | +++ | ++ | ++ |
| 141 | ++ | ++ | ++ |
| 142 | ++ | ++ | ++ |
| 143 | ++ | ++ | ++ |
| 144 | ++ | ++ | ++ |
| 145 | ++ | ++ | ++ |
| 146 | ++ | ++ | ++ |
| 147 | ++ | ++ | ++ |
| 148 | ++ | ++ | ++ |
| 149 | ++ | ++ | ++ |
| 150 | ++ | ++ | ++ |
| 151 | ++ | ++ | ++ |
| 152 | ++ | ++ | ++ |
| 153 | ++ | ++ | ++ |
| 154 | ++ | ++ | ++ |
| 155 | ++ | ++ | ++ |
| 156 | ++ | ++ | + |
| 157 | ++ | ++ | + |
| 158 | ++ | ++ | + |
| 159 | ++ | + | n.d. |
| 160 | +++ | ++ | ++ |
| 161 | +++ | ++ | ++ |
| 162 | +++ | ++ | ++ |
| 163 | +++ | ++ | ++ |
| 164 | +++ | ++ | ++ |
| 165 | +++ | ++ | ++ |
| 166 | +++ | ++ | ++ |
| 167 | +++ | ++ | ++ |
| 168 | +++ | ++ | ++ |
| 169 | +++ | ++ | ++ |
| 170 | +++ | ++ | ++ |
| 171 | +++ | ++ | ++ |
| 172 | +++ | ++ | ++ |
| 173 | +++ | ++ | ++ |
| 174 | +++ | ++ | ++ |
| 175 | +++ | ++ | ++ |
| 176 | +++ | ++ | +++ |
| 177 | +++ | ++ | ++ |
| 178 | +++ | ++ | ++ |
| 179 | +++ | ++ | ++ |
| 180 | +++ | ++ | ++ |
| 181 | +++ | ++ | ++ |
| 182 | +++ | ++ | ++ |
| 183 | +++ | ++ | ++ |
| 184 | +++ | ++ | ++ |
| 185 | +++ | ++ | ++ |
| 186 | +++ | ++ | ++ |
| 187 | +++ | ++ | ++ |
| 188 | +++ | ++ | ++ |
| 189 | +++ | ++ | ++ |
| 190 | +++ | ++ | ++ |
| 191 | +++ | ++ | ++ |
| 192 | +++ | ++ | ++ |
| 193 | +++ | ++ | ++ |
| 194 | +++ | ++ | ++ |
| 195 | ++ | ++ | ++ |
| 196 | ++ | ++ | ++ |
| 197 | ++ | ++ | ++ |
| 198 | ++ | ++ | +++ |
| 199 | ++ | ++ | ++ |
| 200 | ++ | ++ | ++ |
| 201 | ++ | ++ | ++ |
| 202 | ++ | ++ | ++ |
| 203 | ++ | ++ | ++ |
| 204 | ++ | ++ | ++ |
| 205 | ++ | ++ | ++ |
| 206 | ++ | ++ | ++ |
| 207 | ++ | ++ | + |
| 208 | ++ | ++ | + |
| 209 | ++ | ++ | n.d. |
| 210 | ++ | ++ | + |
| 211 | +++ | ++ | ++ |
| 212 | +++ | ++ | ++ |
| 213 | +++ | ++ | ++ |
| 214 | +++ | ++ | ++ |
| 215 | +++ | ++ | ++ |
| 216 | +++ | ++ | ++ |
| 217 | +++ | ++ | ++ |
| 218 | +++ | ++ | ++ |
| 219 | +++ | ++ | ++ |
| 220 | +++ | ++ | ++ |
| 221 | +++ | ++ | ++ |
| 222 | ++ | ++ | ++ |
| 223 | ++ | ++ | ++ |
| 224 | ++ | ++ | ++ |
| 225 | +++ | ++ | ++ |
| 226 | +++ | ++ | ++ |
| 227 | ++ | ++ | + |
| 228 | +++ | ++ | ++ |
| 229 | +++ | ++ | ++ |
| 230 | +++ | ++ | ++ |
| 231 | +++ | ++ | ++ |
| 232 | +++ | ++ | ++ |
| 233 | +++ | ++ | ++ |
| 234 | +++ | ++ | ++ |
| 235 | +++ | ++ | ++ |
| 236 | +++ | ++ | ++ |
| 237 | +++ | ++ | ++ |
| 238 | +++ | ++ | ++ |
| 239 | +++ | ++ | ++ |
| 240 | +++ | ++ | ++ |
| 241 | ++ | ++ | ++ |
| 242 | ++ | ++ | ++ |
| 243 | ++ | ++ | ++ |
| 244 | ++ | ++ | ++ |
| 245 | ++ | ++ | ++ |
| 246 | ++ | ++ | ++ |
| 247 | ++ | ++ | ++ |
| 248 | ++ | ++ | ++ |
| 249 | ++ | ++ | ++ |
| 250 | ++ | ++ | ++ |
| 251 | ++ | ++ | ++ |
| 252 | ++ | ++ | ++ |
| 253 | ++ | ++ | ++ |
| 254 | ++ | ++ | ++ |
| 255 | ++ | ++ | + |
| 256 | ++ | ++ | ++ |
| 257 | ++ | ++ | ++ |
| 258 | +++ | ++ | ++ |
| 259 | +++ | ++ | ++ |
| 260 | ++ | ++ | ++ |
| 261 | +++ | ++ | ++ |
| 262 | +++ | ++ | +++ |
| 263 | ++ | ++ | ++ |
| 264 | +++ | ++ | ++ |
| 265 | +++ | ++ | ++ |
| 266 | + | + | + |
| 267 | +++ | ++ | ++ |
| 268 | +++ | ++ | ++ |
| 269 | +++ | ++ | ++ |
| 270 | +++ | ++ | ++ |
| 271 | +++ | ++ | ++ |
| 272 | +++ | ++ | ++ |
| 273 | +++ | ++ | ++ |
| 274 | +++ | ++ | ++ |
| 275 | +++ | ++ | ++ |
| 276 | +++ | ++ | ++ |
| 277 | ++ | ++ | ++ |
| 278 | +++ | ++ | +++ |
| 279 | +++ | ++ | ++ |
| 280 | +++ | ++ | +++ |
| 281 | +++ | ++ | ++ |
| 282 | +++ | ++ | ++ |
| 283 | +++ | ++ | ++ |
| 284 | +++ | ++ | ++ |
| 285 | +++ | ++ | +++ |
| 286 | +++ | ++ | +++ |
| 287 | +++ | ++ | ++ |
| 288 | +++ | ++ | +++ |
| 289 | +++ | ++ | + |
| 290 | +++ | ++ | ++ |
| 291 | ++ | ++ | ++ |
| 292 | ++ | ++ | ++ |
| 293 | ++ | ++ | ++ |
| 294 | ++ | ++ | ++ |
| 295 | ++ | ++ | + |
| 296 | ++ | ++ | ++ |
| 297 | +++ | ++ | +++ |
| 298 | +++ | ++ | ++ |
| 299 | +++ | ++ | ++ |
| 300 | +++ | ++ | n.d. |
| 301 | +++ | ++ | n.d. |
| 302 | +++ | n.d. | n.d. |
| 303 | +++ | +++ | +++ |
| 304 | ++ | ++ | + |
| 305 | ++ | ++ | ++ |

Note:
+ indicates IC₅₀ values in the range including 1 (μM) to 25 (μM)
++ indicates IC₅₀ values in the range including 0.01 (μM) to less than 1 (μM)
+++ indicates IC₅₀ values <0.01 (μM)
n.d. indicates not determined

1. A compound of formula (I), or a pharmaceutically acceptable salt thereof: 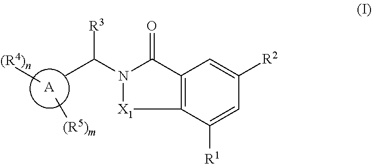
wherein:
X₁ is *—CH₂CH₂—**, *—CH═CH—**, *—N═CH—** or *—CH═N—**, where the * of X₁ indicates the point of attachment to the N and the ** of X₁ indicates the point of attachment to the phenyl ring;
A is selected from 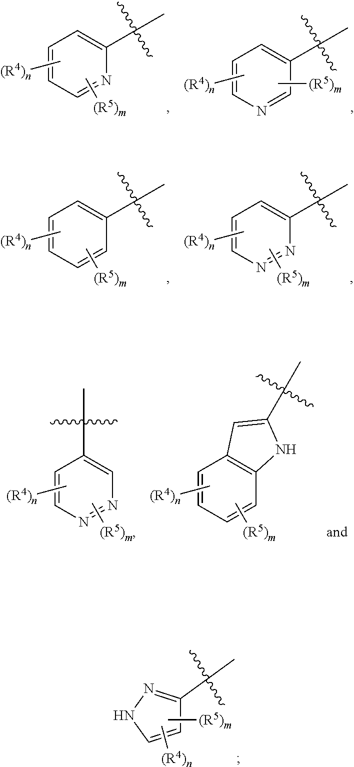
R¹ is selected from H, halo, —R⁶, —CH₂R⁶, —CH₂NR^{1a}R^{1b}, C₁-C₆haloalkyl, a C₁-C₆alkyl substituted with 1 to 2 groups independently selected from —OR^{1a}, —S(═O)₂R¹² and —N(R¹²)₂, and a C₁-C₆haloalkyl substituted with 1 to 2 groups independently selected from C₁-C₆alkyl, —OR^{1a}, —S(═O)₂R¹² and —N(R¹²)₂;
wherein,
R^{1a} is selected from H, C₁-C₆alkyl, C₁-C₆haloalkyl, C₃-C₈cycloalkyl, and a 4-6 membered heterocycloalkyl having 1 to 2 heteroatoms independently selected from N and O, and
R^{1b} is selected from H, C₁-C₆alkyl, C₁-C₆haloalkyl, C₃-C₈cycloalkyl, and a 4-6 membered heterocycloalkyl having 1 to 2 heteroatoms independently selected from N and O;
R² is selected from —(CH₂)R⁷, —(CH₂)₂R⁸ and —(CH₂)₂NR^{2a}R^{2b};
wherein,
R^{2a} is selected from H, C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy, and
R^{2b} is selected from H, C₁-C₆alkyl, C₁-C₆haloalkyl, C₃-C₈cycloalkyl, a 4-6 membered heterocycloalkyl having 1 to 2 heteroatoms independently selected from N and O, and C₁-C₆alkyl substituted with 1 to 2 groups selected from —OH groups and C₁-C₆alkoxy;
R³ is selected from H and C₁-C₆alkyl;
each of R⁴ is independently selected from halo, C₁-C₆alkyl, cyano, C₁-C₆alkoxy, C₁-C₆haloalkoxy and —C(═O)N(R¹²)₂;
R⁵ selected from halo, C₁-C₆alkyl, cyano, C₁-C₆alkoxy, C₁-C₆haloalkoxy and —C(═O)N(R¹²)₂;
R⁶ is selected from phenyl, pyridinonyl, tetrahydropyridinyl, pyridazinonyl, a 4-6 membered heterocycloalkyl having 1 to 2 heteroatoms independently selected from N and O, a 5-6 membered heteroaryl having 1 to 3 heteroatoms independently selected from N, O and S, a 9 to 10 membered heteroaryl having 1-3 heteroatoms independently selected from N, O and S, C₃-C₈cycloalkyl, 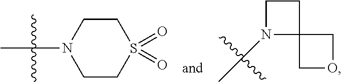 [SMILES: *N1CCS(=O)(=O)CC1 ; *N1CCC12COC2]
wherein the phenyl, pyridinonyl, pyridazinonyl, heterocycloalkyl and heteroaryl of R⁶ are optionally substituted with 1 to 3 R⁹ groups, and wherein each R⁹ is independently selected from C₁-C₆alkyl, C₁-C₆alkoxy, C₁-C₆haloalkyl, C₃-C₈cycloalkyl, halo, cyano, —N(R¹²)₂, —OH, —C(═O)R¹², —S(═O)₂R¹² and a C₁-C₆alkyl substituted with cyano, —N(R¹²)₂ or 1 to 2 —OH groups;
R⁷ is a 5-6 membered heteroaryl having 1 to 3 nitrogen atoms, optionally substituted with 1 to 3 R¹⁰ groups, and wherein each R¹⁰ is independently selected from C₁-C₆alkyl, C₁-C₆alkoxy, C₁-C₆haloalkyl, C₃-C₈cycloalkyl, halo, cyano, —N(R¹²)₂, —OH, and a C₁-C₆alkyl substituted with cyano, —N(R¹²)₂ or 1 to 2 —OH groups;
R⁸ is a 4-6 membered heterocycloalkyl having 1 to 2 heteroatoms independently selected from N and O, optionally substituted with 1 to 2 R¹¹ groups, and wherein each R¹¹ is independently selected from C₁-C₆alkyl, C₁-C₆alkoxy, C₁-C₆haloalkyl, C₃-C₈cycloalkyl, halo, cyano, —N(R¹²)₂, —OH, and a C₁-C₆alkyl substituted with cyano, —N(R¹²)₂ or 1 to 2 —OH groups;
each R¹² is independently selected from H and C₁-C₆alkyl;
m is 0 or 1, and
n is 0, 1 or 2.

2. The compound according to claim 1, or a pharmaceutically acceptable salt thereof, wherein the compound of formula (I) has the structure of formula (Ia), formula (Ib), formula (Ic) or formula (Id), or pharmaceutically acceptable salt thereof,

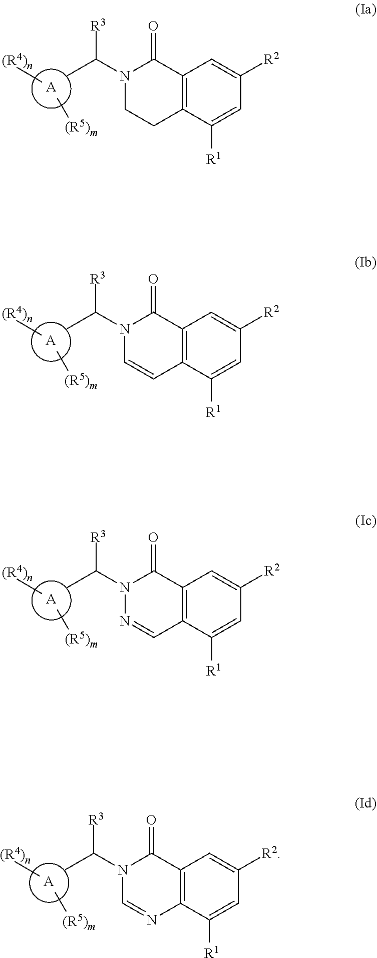

3. The compound of claim 1, or a pharmaceutically acceptable salt thereof, wherein:
A is

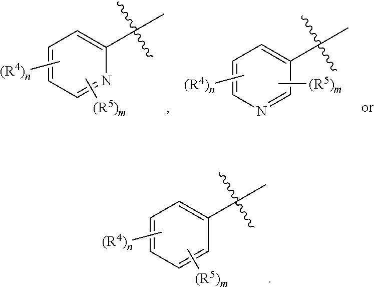

4. The compound of claim 1, or a pharmaceutically acceptable salt thereof, wherein each of R⁴ is independently selected from methoxy, ethoxy, cyano, fluoro, chloro, —OCHF₂, methyl, ethyl, —C(═O)NH₂.

5. The compound of claim 1, or a pharmaceutically acceptable salt thereof, wherein R⁵ is independently selected from methoxy, ethoxy, cyano, fluoro, chloro, —OCHF₂, methyl, ethyl and —C(═O)NH₂.

6. The compound of claim 1, or a pharmaceutically acceptable salt thereof, wherein R³ is H or methyl.

7. The compound of claim 1, or a pharmaceutically acceptable salt thereof, wherein R¹ is selected from H, —R⁶, and —CH₂NR^{1a}R^{1b}.

8. The compound of claim 1, or a pharmaceutically acceptable salt thereof, wherein:
R¹ is —CH₂NR^{1a}R^{1b};
R^{1a} is selected from C₁-C₂alkyl, C₁-C₂haloalkyl, C₃-C₅cycloalkyl, and a 4 membered heterocycloalkyl having 1 to 2 heteroatoms independently selected from N and O, and
R^{1b} is selected from C₁-C₂alkyl, C₁-C₂haloalkyl, C₃-C₅cycloalkyl, and a 4 membered heterocycloalkyl having 1 to 2 heteroatoms independently selected from N and O.

9. The compound of claim 1, or a pharmaceutically acceptable salt thereof, wherein:
R¹ is —CH₂NR^{1a}R^{1b};
R^{1a} is selected from methyl, —CH₂CHF₂, —CH₂CF₃, —CH₂CH₂F, cyclopropyl, and an oxetanyl, and
R^{1b} is selected from methyl, —CH₂CHF₂, —CH₂CF₃, —CH₂CH₂F, cyclopropyl, and an oxetanyl.

10. The compound of claim 1, or a pharmaceutically acceptable salt thereof, wherein:
R¹ is —CH₂NR^{1a}R^{1b};
R^{1a} is selected from methyl, —CH₂CHF₂, —CH₂CF₃, —CH₂CH₂F, cyclopropyl, and an oxetanyl, and
R^{1b} is methyl.

11. The compound of claim 1, or a pharmaceutically acceptable salt thereof, wherein:
R¹ is selected from —R⁶ and —CH₂R⁶.
R⁶ is selected from phenyl, cyclopropyl. pyridinonyl, tetrahydropyridinyl, pyridazinonyl, morpholinyl, pyrrolidinyl, azetadinyl, cyclopropyl pyridinyl, pyrazolyl, pyrimidinyl, imidazolyl, pyridazinyl, oxazolopyridinyl, imidazopyridinyl or triazolyl, each of which is optionally substituted with 1 to 3 R⁹ groups, and
each R⁹ is independently selected from fluoro, chloro, methyl, ethyl, methoxy, cyano, cyclopropyl, —OH, —CF₃, —CHF₂, —CH₂OH, —CHOHCH₃, —CH₂CH₂OH, —C(CH₃)₂OH, —NH₂, —C(═O)CH₃, —CH₂CN, —CH₂N(CH₃)₂, —NHCH₃ and S(═O)₂CH₃.

12. The compound of claim 1, or a pharmaceutically acceptable salt thereof, wherein:
R¹ is selected from —R⁶ and —CH₂R⁶;
R⁶ is selected from azetadinyl and pyridyl, each of which is optionally substituted with 1 to 3 R⁹ groups, and
each R⁹ is independently selected from fluoro, —NH₂, and methyl.

13. The compound of claim 1, or a pharmaceutically acceptable salt thereof, wherein R¹ is selected from H, bromo, chloro, —CHF₂, —CH(OH)CH(CH₃)₂, —CH(OH)CH(CH₃)₃, —C(CH₃)(OH)(CF₃), CH(OH)CH(CH₃)CF₃, and —CH(OH)CF₃.

14. The compound of claim 1, or a pharmaceutically acceptable salt thereof, wherein:
R² is selected from —(CH₂)R⁷ and —(CH₂)₂NR^{2a}R^{2b}.

15. The compound of claim 1, or a pharmaceutically acceptable salt thereof, wherein:
R² is —(CH₂)₂NR^{2a}R^{2b};
R^{2a} is selected from H, methyl, ethyl, cyclopropyl, oxetanyl, —CH₂CH₂F, —CH₂CH₂OH, —CH₂CH₂OCH₃ and —CH₂CH(OH)CH₃, and
R^{2b} is selected from H, methyl, ethyl, cyclopropyl, oxetanyl, —CH₂CH₂F, —CH₂CH₂OH, —CH₂CH₂OCH₃ and —CH₂CH(OH)CH₃.

16. The compound of claim 1, or a pharmaceutically acceptable salt thereof, wherein:
R² is —(CH₂)₂NR^{2a}R^{2b};
R^{2a} is selected from methyl, ethyl, cyclopropyl, oxetanyl, —CH₂CH₂F, —CH₂CH₂OH, —CH₂CH₂OCH₃ and —CH₂CH(OH)CH₃, and
R^{2b} is selected from methyl.

17. The compound of claim 1, or a pharmaceutically acceptable salt thereof, wherein:
R² is —(CH₂)₂NR^{2a}R^{2b};
R^{2a} is selected from ethyl, and
R^{2b} is selected from methyl.

18. The compound of claim 1, or a pharmaceutically acceptable salt thereof, wherein:
R² is —(CH₂)R⁷;
R⁷ is azetidinyl, pyrrolidinyl or imidazolyl, each of which is optionally substituted with 1 to 3 R¹⁰ groups, and
each R¹⁰ is independently selected from fluoro, methyl, methoxy, —OH, —CH₂OH, and —NHCH₃.

19. The compound of claim 1, or a pharmaceutically acceptable salt thereof, wherein:
R² is —(CH₂)R⁷, and
R⁷ is imidazolyl substituted with methyl or —NHCH₃.

20. The compound of claim 1, or a pharmaceutically acceptable salt thereof, having the structure of formula (Ie), formula (If) or formula (Ig), or pharmaceutically acceptable salt thereof, 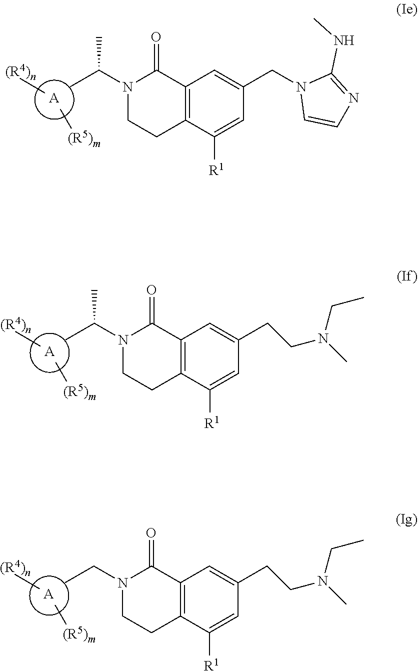
wherein:
A is 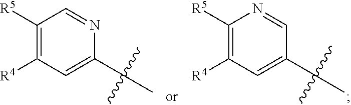
R¹ is selected from H, —R⁶ and —CH₂NR^{1a}R^{2b};
wherein,
R^{1a} is C₁-C₆alkyl, and
R^{1b} is C₁-C₆alkyl;
R² is selected from —(CH₂)R⁷ and —(CH₂)₂NR^{2a}R^{2b};
wherein,
R^{2a} is C₁-C₆alkyl and
R^{2b} is C₁-C₆alkyl;
R⁴ is C₁-C₆alkoxy;
R⁵ is halo or cyano;
R⁶ is pyridinonyl or a 5-6 membered heteroaryl having 1 to 3 heteroatoms independently selected from N, O and S, wherein the pyridinonyl and heteroaryl of R⁶ are substituted with 1 to 3 R⁹ groups, and wherein each R⁹ is independently selected from C₁-C₆alkyl, C₁-C₆haloalkyl, halo and —N(R¹²)₂;
R⁷ is a 5-6 membered heteroaryl having 1 to 3 nitrogen atoms, substituted with 1 to 3 R¹⁰ groups, and wherein each R¹⁰ is independently selected from C₁-C₆alkyl and —N(R¹²)₂,
and
each R¹² is independently selected from H and C₁-C₆alkyl.

21. The compound of claim 1 selected from:
6-(1-(5-bromo-7-((2-(methylamino)-1H-imidazol-1-yl)methyl)-1-oxo-3,4 dihydroisoquinolin-2(1H)-yl)ethyl)-4-ethoxynicotinonitrile;
5-((5-bromo-7-((2-(methylamino)-1H-imidazol-1-yl)methyl)-1-oxo-3,4-dihydroisoquinolin-2(1H)-yl)methyl)-3-ethoxypicolinonitrile;
(S)-6-(1-(5-chloro-7-((2-(methylamino)-1H-imidazol-1-yl)methyl)-1-oxo-3,4-dihydroisoquinolin-2(1H)-yl)ethyl)-4-ethoxynicotinonitrile;
(R)-5-(1-(5-chloro-7-((2-(methylamino)-1H-imidazol-1-yl)methyl)-1-oxo-3,4-dihydroisoquinolin-2(1H)-yl)ethyl)-3-ethoxypicolinonitrile;
(S)-5-chloro-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
5-((5-chloro-7-((2-(methylamino)-1H-imidazol-1-yl)methyl)-1-oxo-3,4-dihydroisoquinolin-2(1H)-yl)methyl)-3-ethoxypicolinonitrile;
(S)-2-(1-(5-fluoro-4-methoxypyridin-2-yl)ethyl)-7-((2-(methylamino)-1H-imidazol-1-yl)methyl)-5-(morpholinomethyl)-3,4-dihydroisoquinolin-1(2H)-one;
(S)-2-(1-(6-fluoro-5-methoxypyridin-3-yl)ethyl)-7-((2-(methylamino)-1H-imidazol-1-yl)methyl)-5-(morpholinomethyl)-3,4-dihydroisoquinolin-1(2H)-one;
4-ethoxy-6-((S)-1-(5-(((S)-3-fluoropyrrolidin-1-yl)methyl)-7-((2-(methylamino)-1H-imidazol-1-yl)methyl)-1-oxo-3,4-dihydroisoquinolin-2(1H)-yl)ethyl)nicotinonitrile;
(S)-2-(1-(4-ethoxy-5-fluoropyridin-2-yl)ethyl)-5-((methyl(oxetan-3-yl)amino)methyl)-7-((2-(methylamino)-1H-imidazol-1-yl)methyl)-3,4-dihydroisoquinolin-1(2H)-one;
4-ethoxy-6-((S)-1-(5-(((R)-3-fluoropyrrolidin-1-yl)methyl)-7-((2-(methylamino)-1H-imidazol-1-yl)methyl)-1-oxo-3,4-dihydroisoquinolin-2(1H)-yl)ethyl)nicotinonitrile;
(S)-4-ethoxy-6-(1-(7-((2-(methylamino)-1H-imidazol-1-yl)methyl)-5-(morpholinomethyl)-1-oxo-3,4-dihydroisoquinolin-2(1H)-yl)ethyl)nicotinonitrile;
(S)-2-(1-(5-fluoro-4-methoxypyridin-2-yl)ethyl)-5-((methyl(oxetan-3-yl)amino)methyl)-7-((2-(methylamino)-1H-imidazol-1-yl)methyl)-3,4-dihydroisoquinolin-1(2H)-one;
2-((S)-1-(4-ethoxy-5-fluoropyridin-2-yl)ethyl)-5-(((R)-3-fluoropyrrolidin-1-yl)methyl)-7-((2-(methylamino)-1H-imidazol-1-yl)methyl)-3,4-dihydroisoquinolin-1(2H)-one;
(S)-5-((cyclopropyl(methyl)amino)methyl)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
2-((S)-1-(5-fluoro-4-methoxypyridin-2-yl)ethyl)-5-(((R)-3-fluoropyrrolidin-1-yl)methyl)-7-((2-(methylamino)-1H-imidazol-1-yl)methyl)-3,4-dihydroisoquinolin-1(2H)-one;
(S)-5-((6-oxa-1-azaspiro[3.3]heptan-1-yl)methyl)-2-(1-(5-fluoro-4-methoxypyridin-2-yl)ethyl)-7-((2-(methylamino)-1H-imidazol-1-yl)methyl)-3,4-dihydroisoquinolin-1(2H)-one;
(S)-6-(1-(5-((cyclopropyl(methyl)amino)methyl)-7-((2-(methylamino)-1H-imidazol-1-yl)methyl)-1-oxo-3,4-dihydroisoquinolin-2(1H)-yl)ethyl)-4-ethoxynicotinonitrile;
(S)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(R)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(S)-2-(1-(5-chloro-4-ethoxypyridin-2-yl)ethyl)-5-((dimethylamino)methyl)-7-((2-(methylamino)-1H-imidazol-1-yl)methyl)-3,4-dihydroisoquinolin-1(2H)-one;
(S)-5-((cyclopropyl(methyl)amino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(S)-2-(1-(5-chloro-4-ethoxypyridin-2-yl)ethyl)-5-((cyclopropyl(methyl)amino)methyl)-7-((2-(methylamino)-1H-imidazol-1-yl)methyl)-3,4-dihydroisoquinolin-1(2H)-one;
(S)-2-(1-(5-chloro-4-ethoxypyridin-2-yl)ethyl)-7-((2-(methylamino)-1H-imidazol-1-yl)methyl)-5-(morpholinomethyl)-3,4-dihydroisoquinolin-1(2H)-one;
5-((5-((dimethylamino)methyl)-7-((2-(methylamino)-1H-imidazol-1-yl)methyl)-1-oxo-3,4-dihydroisoquinolin-2(1H)-yl)methyl)-3-ethoxypicolinonitrile;
3-ethoxy-5-((7-((2-(methylamino)-1H-imidazol-1-yl)methyl)-5-((3-methylazetidin-1-yl)methyl)-1-oxo-3,4-dihydroisoquinolin-2(1H)-yl)methyl)picolinonitrile;
5-((5-((cyclopropyl(methyl)amino)methyl)-7-((2-(methylamino)-1H-imidazol-1-yl)methyl)-1-oxo-3,4-dihydroisoquinolin-2(1H)-yl)methyl)-3-ethoxypicolinonitrile;
(S)-2-(1-(4-ethoxy-5-fluoropyridin-2-yl)ethyl)-7-((2-(methylamino)-1H-imidazol-1-yl)methyl)-5-(morpholinomethyl)-3,4-dihydroisoquinolin-1(2H)-one;
(S)-4-ethoxy-6-(1-(5-((3-fluoroazetidin-1-yl)methyl)-7-((2-(methylamino)-1H-imidazol-1-yl)methyl)-1-oxo-3,4-dihydroisoquinolin-2(1H)-yl)ethyl)nicotinonitrile;
(S)-5-((dimethylamino)methyl)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
2-((5-fluoro-4-methoxypyridin-2-yl)methyl)-5-((3-fluoroazetidin-1-yl)methyl)-7-((2-(methylamino)-1H-imidazol-1-yl)methyl)-3,4-dihydroisoquinolin-1(2H)-one;
(S)-2-(1-(5-fluoro-4-methoxypyridin-2-yl)ethyl)-5-((3-methoxyazetidin-1-yl)methyl)-7-((2-(methylamino)-1H-imidazol-1-yl)methyl)-3,4-dihydroisoquinolin-1(2H)-one;
(S)-5-(1-(5-((dimethylamino)methyl)-7-((2-(methylamino)-1H-imidazol-1-yl)methyl)-1-oxo-3,4-dihydroisoquinolin-2(1H)-yl)ethyl)-3-ethoxypicolinonitrile;
2-((6-fluoro-5-methoxypyridin-3-yl)methyl)-5-((3-fluoroazetidin-1-yl)methyl)-7-((2-(methylamino)-1H-imidazol-1-yl)methyl)-3,4-dihydroisoquinolin-1(2H)-one;
(S)-5-((3,3-difluoroazetidin-1-yl)methyl)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
(S)-5-(((2,2-difluoroethyl)(methyl)amino)methyl)-2-(1-(5-fluoro-4-methoxypyridin-2-yl)ethyl)-7-((2-(methylamino)-1H-imidazol-1-yl)methyl)-3,4-dihydroisoquinolin-1(2H)-one;
2-((S)-1-(5-fluoro-4-methoxypyridin-2-yl)ethyl)-5-(((R)-3-methoxypyrrolidin-1-yl)methyl)-7-((2-(methylamino)-1H-imidazol-1-yl)methyl)-3,4-dihydroisoquinolin-1(2H)-one;
2-((S)-1-(4-ethoxy-5-fluoropyridin-2-yl)ethyl)-5-(((S)-3-fluoropyrrolidin-1-yl)methyl)-7-((2-(methylamino)-1H-imidazol-1-yl)methyl)-3,4-dihydroisoquinolin-1(2H)-one;
5-((3,3-difluoroazetidin-1-yl)methyl)-2-((5-fluoro-4-methoxypyridin-2-yl)methyl)-7-((2-(methylamino)-1H-imidazol-1-yl)methyl)-3,4-dihydroisoquinolin-1(2H)-one;
(S)-2-(1-(4-ethoxy-5-fluoropyridin-2-yl)ethyl)-5-((3-fluoroazetidin-1-yl)methyl)-7-((2-(methylamino)-1H-imidazol-1-yl)methyl)-3,4-dihydroisoquinolin-1(2H)-one;
(S)-6-(1-(5-((3,3-difluoroazetidin-1-yl)methyl)-7-((2-(methylamino)-1H-imidazol-1-yl)methyl)-1-oxo-3,4-dihydroisoquinolin-2(1H)-yl)ethyl)-4-ethoxynicotinonitrile;
(S)-3-ethoxy-5-(1-(5-((3-fluoroazetidin-1-yl)methyl)-7-((2-(methylamino)-1H-imidazol-1-yl)methyl)-1-oxo-3,4-dihydroisoquinolin-2(1H)-yl)ethyl)picolinonitrile;
(S)-2-(1-(5-fluoro-4-methoxypyridin-2-yl)ethyl)-5-((3-fluoroazetidin-1-yl)methyl)-7-((2-(methylamino)-1H-imidazol-1-yl)methyl)-3,4-dihydroisoquinolin-1(2H)-one;
(S)-2-(1-(6-fluoro-5-methoxypyridin-3-yl)ethyl)-5-((3-fluoroazetidin-1-yl)methyl)-7-((2-(methylamino)-1H-imidazol-1-yl)methyl)-3,4-dihydroisoquinolin-1(2H)-one;
2-((S)-1-(5-fluoro-4-methoxypyridin-2-yl)ethyl)-5-(((S)-3-fluoropyrrolidin-1-yl)methyl)-7-((2-(methylamino)-1H-imidazol-1-yl)methyl)-3,4-dihydroisoquinolin-1(2H)-one;
(S)-6-(1-(5-((dimethylamino)methyl)-7-((2-(methylamino)-1H-imidazol-1-yl)methyl)-1-oxo-3,4-dihydroisoquinolin-2(1H)-yl)ethyl)-4-methoxynicotinonitrile;
5-((5-((3,3-difluoroazetidin-1-yl)methyl)-7-((2-(methylamino)-1H-imidazol-1-yl)methyl)-1-oxo-3,4-dihydroisoquinolin-2(1H)-yl)methyl)-3-ethoxypicolinonitrile;
(S)-2-(1-(5-fluoro-4-methoxypyridin-2-yl)ethyl)-5-((methyl(2,2,2-trifluoroethyl)amino)methyl)-7-((2-(methylamino)-1H-imidazol-1-yl)methyl)-3,4-dihydroisoquinolin-1(2H)-one;
(S)-5-((3,3-difluoroazetidin-1-yl)methyl)-2-(1-(5-fluoro-4-methoxypyridin-2-yl)ethyl)-7-((2-(methylamino)-1H-imidazol-1-yl)methyl)-3,4-dihydroisoquinolin-1(2H)-one;
(S)-2-(1-(5-fluoro-4-methoxypyridin-2-yl)ethyl)-5-(((2-fluoroethyl)(methyl)amino)methyl)-7-((2-(methylamino)-1H-imidazol-1-yl)methyl)-3,4-dihydroisoquinolin-1(2H)-one;
(S)-6-(1-(5-((dimethylamino)methyl)-7-((2-(methylamino)-1H-imidazol-1-yl)methyl)-1-oxo-3,4-dihydroisoquinolin-2(1H)-yl)ethyl)-4-ethoxynicotinonitrile;
(S)-2-(1-(5-fluoro-4-methoxypyridin-2-yl)ethyl)-5-((3-hydroxyazetidin-1-yl)methyl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-(3-methoxybenzyl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-(4-fluoro-3-methoxybenzyl)-7-((2-(methylamino)-1H-imidazol-1-yl)methyl)-3,4-dihydroisoquinolin-1(2H)-one;
2-(4-fluoro-3-methoxybenzyl)-5-(1-methyl-3-(trifluoromethyl)-1H-pyrazol-4-yl)-7-((2-(methylamino)-1H-imidazol-1-yl)methyl)-3,4-dihydroisoquinolin-1(2H)-one;
2-(4-fluoro-3-methoxybenzyl)-5-(3-methyl-1H-pyrazol-4-yl)-7-((2-(methylamino)-1H-imidazol-1-yl)methyl)-3,4-dihydroisoquinolin-1(2H)-one;
(S)-5-(2-chloro-5-(hydroxymethyl)pyridin-3-yl)-2-(1-(4-fluoro-3-methoxyphenyl)ethyl)-7-((2-(methylamino)-1H-imidazol-1-yl)methyl)-3,4-dihydroisoquinolin-1(2H)-one;
(R)-5-(2-chloro-5-(hydroxymethyl)pyridin-3-yl)-2-(1-(4-fluoro-3-methoxyphenyl)ethyl)-7-((2-(methylamino)-1H-imidazol-1-yl)methyl)-3,4-dihydroisoquinolin-1(2H)-one;
5-(1,3-dimethyl-1H-pyrazol-4-yl)-2-(4-fluoro-3-methoxybenzyl)-7-((2-(methylamino)-1H-imidazol-1-yl)methyl)-3,4-dihydroisoquinolin-1(2H)-one;
(S)-2-(1-(5-fluoro-4-methoxypyridin-2-yl)ethyl)-7-((2-(methylamino)-1H-imidazol-1-yl)methyl)-5-(pyrimidin-5-yl)-3,4-dihydroisoquinolin-1(2H)-one;
2-((5-fluoro-4-methoxypyridin-2-yl)methyl)-5-(1-methyl-1H-pyrazol-5-yl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-(2,4-difluoro-5-methoxybenzyl)-7-((2-(methylamino)-1H-imidazol-1-yl)methyl)-3,4-dihydroisoquinolin-1(2H)-one;
(S)-5-(1,3-dimethyl-1H-pyrazol-4-yl)-2-(1-(5-fluoro-4-methoxypyridin-2-yl)ethyl)-7-((2-(methylamino)-1H-imidazol-1-yl)methyl)-3,4-dihydroisoquinolin-1(2H)-one;
(S)-2-(1-(5-fluoro-4-methoxypyridin-2-yl)ethyl)-7-((2-(methylamino)-1H-imidazol-1-yl)methyl)-5-(1H-pyrazol-5-yl)-3,4-dihydroisoquinolin-1(2H)-one;
2-((5-fluoro-4-methoxypyridin-2-yl)methyl)-5-(1-methyl-3-(trifluoromethyl)-1H-pyrazol-4-yl)-7-((2-(methylamino)-1H-imidazol-1-yl)methyl)-3,4-dihydroisoquinolin-1(2H)-one;
5-(2-chloro-5-(hydroxymethyl)phenyl)-2-(4-fluoro-3-methoxybenzyl)-7-((2-(methylamino)-1H-imidazol-1-yl)methyl)-3,4-dihydroisoquinolin-1(2H)-one;
5-(3-(difluoromethyl)-1-methyl-1H-pyrazol-4-yl)-2-(4-fluoro-3-methoxybenzyl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-(3-chloro-5-fluorobenzyl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-((5-fluoro-4-methoxypyridin-2-yl)methyl)-7-((2-(methylamino)-1H-imidazol-1-yl)methyl)-3,4-dihydroisoquinolin-1(2H)-one;
5-((5-(2-chloro-5-(hydroxymethyl)pyridin-3-yl)-7-((2-(methylamino)-1H-imidazol-1-yl)methyl)-1-oxo-3,4-dihydroisoquinolin-2(1H)-yl)methyl)-2-fluorobenzonitrile;
5-(2-chloro-5-(hydroxymethyl)pyridin-3-yl)-2-(3,4-difluorobenzyl)-7-((2-(methylamino)-1H-imidazol-1-yl)methyl)-3,4-dihydroisoquinolin-1(2H)-one;
(R)-5-(1,3-dimethyl-1H-pyrazol-4-yl)-2-(1-(5-fluoro-4-methoxypyridin-2-yl)ethyl)-7-((2-(methylamino)-1H-imidazol-1-yl)methyl)-3,4-dihydroisoquinolin-1(2H)-one;
2-(4-fluoro-3-methoxybenzyl)-5-(1-methyl-1H-pyrazol-5-yl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-(3-(difluoromethoxy)benzyl)-7-((2-(methylamino)-1H-imidazol-1-yl)methyl)-3,4-dihydroisoquinolin-1(2H)-one;
5-(2-chloro-5-(hydroxymethyl)pyridin-3-yl)-2-(3-(difluoromethoxy)-4-fluorobenzyl)-7-((2-(methylamino)-1H-imidazol-1-yl)methyl)-3,4-dihydroisoquinolin-1(2H)-one;
(R)-5-(2-chloro-5-(1-hydroxyethyl)pyridin-3-yl)-2-(4-fluoro-3-methoxybenzyl)-7-((2-(methylamino)-1H-imidazol-1-yl)methyl)-3,4-dihydroisoquinolin-1(2H)-one;
(S)-5-(2-chloro-5-(1-hydroxyethyl)pyridin-3-yl)-2-(4-fluoro-3-methoxybenzyl)-7-((2-(methylamino)-1H-imidazol-1-yl)methyl)-3,4-dihydroisoquinolin-1(2H)-one;
4-(2-(4-fluoro-3-methoxybenzyl)-7-((2-(methylamino)-1H-imidazol-1-yl)methyl)-1-oxo-1,2,3,4-tetrahydroisoquinolin-5-yl)-1-methyl-1H-pyrazole-3-carbonitrile;
(S)-5-cyclopropyl-2-(1-(6-fluoro-5-methoxypyridin-3-yl)ethyl)-7-((2-(methylamino)-1H-imidazol-1-yl)methyl)-3,4-dihydroisoquinolin-1(2H)-one;
5-cyclopropyl-2-((6-fluoro-5-methoxypyridin-3-yl)methyl)-7-((2-(methylamino)-1H-imidazol-1-yl)methyl)-3,4-dihydroisoquinolin-1(2H)-one;
(S)-5-(1-(5-cyclopropyl-7-((2-(methylamino)-1H-imidazol-1-yl)methyl)-1-oxo-3,4-dihydroisoquinolin-2(1H)-yl)ethyl)-3-ethoxypicolinonitrile;
(S)-5-cyclopropyl-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(R)-6-(1-(5-cyclopropyl-7-((2-(methylamino)-1H-imidazol-1-yl)methyl)-1-oxo-3,4-dihydroisoquinolin-2(1H)-yl)ethyl)-4-ethoxynicotinonitrile;
(S)-6-(1-(5-cyclopropyl-7-((2-(methylamino)-1H-imidazol-1-yl)methyl)-1-oxo-3,4-dihydroisoquinolin-2(1H)-yl)ethyl)-4-ethoxynicotinonitrile;
5-((5-cyclopropyl-7-((2-(methylamino)-1H-imidazol-1-yl)methyl)-1-oxo-3,4-dihydroisoquinolin-2(1H)-yl)methyl)-3-ethoxypicolinonitrile;
(S)-5-cyclopropyl-2-(1-(5-fluoro-4-methoxypyridin-2-yl)ethyl)-7-((2-(methylamino)-1H-imidazol-1-yl)methyl)-3,4-dihydroisoquinolin-1(2H)-one;
5-cyclopropyl-2-((1-ethyl-5-methoxy-1H-pyrazol-3-yl)methyl)-7-((2-(methylamino)-1H-imidazol-1-yl)methyl)-3,4-dihydroisoquinolin-1(2H)-one;
5-cyclopropyl-2-((5-ethoxy-1-methyl-1H-pyrazol-3-yl)methyl)-7-((2-(methylamino)-1H-imidazol-1-yl)methyl)-3,4-dihydroisoquinolin-1(2H)-one;
(S)-2-(1-(4-ethoxy-5-fluoropyridin-2-yl)ethyl)-7-((2-(methylamino)-1H-imidazol-1-yl)methyl)-5-(pyrrolidin-1-yl)-3,4-dihydroisoquinolin-1(2H)-one;
(S)-2-(1-(5-fluoro-4-methoxypyridin-2-yl)ethyl)-7-((2-(methylamino)-1H-imidazol-1-yl)methyl)-5-(pyrrolidin-1-yl)-3,4-dihydroisoquinolin-1(2H)-one;
(S)-2-(1-(5-fluoro-4-methoxypyridin-2-yl)ethyl)-5-(3-methoxyazetidin-1-yl)-7-((2-(methylamino)-1H-imidazol-1-yl)methyl)-3,4-dihydroisoquinolin-1(2H)-one;
(S)-5-(difluoromethyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(S)-5-(difluoromethyl)-2-(1-(5-fluoro-4-methoxypyridin-2-yl)ethyl)-7-((2-(methylamino)-1H-imidazol-1-yl)methyl)-3,4-dihydroisoquinolin-1(2H)-one;
(S)-5-(difluoromethyl)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
(S)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
(R)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
2-(1-(6-fluoro-5-methoxypyridin-3-yl)ethyl)-7-((2-(methylamino)-1H-imidazol-1-yl)methyl)-3,4-dihydroisoquinolin-1(2H)-one;
(S)-2-(1-(6-chloro-5-ethoxypyridin-3-yl)ethyl)-7-((2-(methylamino)-1H-imidazol-1-yl)methyl)-3,4-dihydroisoquinolin-1(2H)-one;
3-ethoxy-5-((7-((2-(methylamino)-1H-imidazol-1-yl)methyl)-1-oxo-3,4-dihydroisoquinolin-2(1H)-yl)methyl)picolinonitrile;
(S)-3-ethoxy-5-(1-(7-((2-(methylamino)-1H-imidazol-1-yl)methyl)-1-oxo-3,4-dihydroisoquinolin-2(1H)-yl)ethyl)picolinonitrile;
5-(2-chloro-5-(hydroxymethyl)pyridin-3-yl)-2-(3-(difluoromethoxy)benzyl)-7-((2-(methylamino)-1H-imidazol-1-yl)methyl)isoquinolin-1(2H)-one;
5-bromo-2-(4-fluoro-3-methoxybenzyl)-7-((2-(methylamino)-1H-imidazol-1-yl)methyl)isoquinolin-1(2H)-one;
5-(2-chloro-5-(hydroxymethyl)pyridin-3-yl)-2-(3-methoxybenzyl)-7-((2-(methylamino)-1H-imidazol-1-yl)methyl)isoquinolin-1(2H)-one;
5-(2-chloro-5-(hydroxymethyl)pyridin-3-yl)-2-(4-fluoro-3-methoxybenzyl)-7-((2-(methylamino)-1H-imidazol-1-yl)methyl)isoquinolin-1(2H)-one;
(S)-5-(2-chloro-5-(hydroxymethyl)pyridin-3-yl)-2-(1-(3-methoxyphenyl)ethyl)-7-((2-(methylamino)-1H-imidazol-1-yl)methyl)isoquinolin-1(2H)-one;
(R)-5-(2-chloro-5-(hydroxymethyl)pyridin-3-yl)-2-(1-(3-methoxyphenyl)ethyl)-7-((2-(methylamino)-1H-imidazol-1-yl)methyl)isoquinolin-1(2H)-one;
5-(2-chloro-5-(hydroxymethyl)pyridin-3-yl)-2-(3-fluoro-4-methoxybenzyl)-7-((2-(methylamino)-1H-imidazol-1-yl)methyl)isoquinolin-1(2H)-one;
5-(2-chloro-5-(hydroxymethyl)pyridin-3-yl)-2-(4-methoxybenzyl)-7-((2-(methylamino)-1H-imidazol-1-yl)methyl)isoquinolin-1(2H)-one;
5-(2-chloro-5-(1-hydroxyethyl)pyridin-3-yl)-2-(4-fluoro-3-methoxybenzyl)-7-((2-(methylamino)-1H-imidazol-1-yl)methyl)isoquinolin-1(2H)-one;
5-(2-chloro-5-(hydroxymethyl)pyridin-3-yl)-2-((4-methyl-1H-indol-2-yl)methyl)-7-((2-(methylamino)-1H-imidazol-1-yl)methyl)isoquinolin-1(2H)-one;
2-((1H-indol-2-yl)methyl)-5-(2-chloro-5-(hydroxymethyl)pyridin-3-yl)-7-((2-(methylamino)-1H-imidazol-1-yl)methyl)isoquinolin-1(2H)-one;
2-(1-(6-fluoro-5-methoxypyridin-3-yl)ethyl)-7-((2-(methylamino)-1H-imidazol-1-yl)methyl)phthalazin-1(2H)-one;
3-(1-(6-fluoro-5-methoxypyridin-3-yl)ethyl)-6-((2-(methylamino)-1H-imidazol-1-yl)methyl)quinazolin-4(3H)-one;
(S)-4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile;
(R)-4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile;
4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile;
6-((5-(2-chloro-5-methylpyridin-3-yl)-7-(2-(ethyl(methyl)amino)ethyl)-1-oxo-3,4-dihydroisoquinolin-2(1H)-yl)methyl)-4-ethoxynicotinonitrile;
(S)-5-(1,3-dimethyl-1H-pyrazol-4-yl)-7-(2-(ethyl(methyl)amino)ethyl)-2-(1-(5-fluoro-4-methoxypyridin-2-yl)ethyl)-3,4-dihydroisoquinolin-1(2H)-one;
(S)-6-(1-(5-(2,5-dimethylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(1-(2-hydroxyethyl)-3-methyl-1H-pyrazol-4-yl)-1-oxo-3,4-dihydroisoquinolin-2(1H)-yl)ethyl)nicotinonitrile;
(S)-4-ethoxy-6-(1-(7-(2-(ethyl(methyl)amino)ethyl)-5-(3-methyl-1H-pyrazol-4-yl)-1-oxo-3,4-dihydroisoquinolin-2(1H)-yl)ethyl)nicotinonitrile;
(S)-4-ethoxy-6-(1-(7-(2-(ethyl(methyl)amino)ethyl)-5-(2-fluoro-3-methylpyridin-4-yl)-1-oxo-3,4-dihydroisoquinolin-2(1H)-yl)ethyl)nicotinonitrile;
(S)-6-(1-(5-(2-chloro-5-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(2-(difluoromethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-5-(2-chloro-5-(hydroxymethyl)pyridin-3-yl)-2-(1-(4-ethoxy-5-fluoropyridin-2-yl)ethyl)-7-(2-(ethyl(methyl)amino)ethyl)-3,4-dihydroisoquinolin-1(2H)-one;
(S)-2-(1-(5-chloro-4-ethoxypyridin-2-yl)ethyl)-5-(1,3-dimethyl-1H-pyrazol-4-yl)-7-(2-(ethyl(methyl)amino)ethyl)-3,4-dihydroisoquinolin-1(2H)-one;
(S)-6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(R)-6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-5-(1,3-dimethyl-1H-pyrazol-4-yl)-7-(2-(ethyl(methyl)amino)ethyl)-2-(1-(6-fluoro-5-methoxypyridin-3-yl)ethyl)-3,4-dihydroisoquinolin-1(2H)-one;
(S)-6-(1-(5-(2-chloro-5-(hydroxymethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-1-oxo-5-(1,4,5-trimethyl-6-oxo-1,6-dihydropyridin-3-yl)-3,4-dihydroisoquinolin-2(1H)-yl)ethyl)nicotinonitrile;
(S)-6-(1-(5-(3-(difluoromethyl)-1-methyl-1H-pyrazol-4-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(1-ethyl-3-methyl-1H-pyrazol-4-yl)-1-oxo-3,4-dihydroisoquinolin-2(1H)-yl)ethyl)nicotinonitrile;
(S)-6-(1-(5-(2-(difluoromethyl)-5-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
6-((5-(1,3-dimethyl-1H-pyrazol-4-yl)-7-(2-(ethyl(methyl)amino)ethyl)-1-oxo-3,4-dihydroisoquinolin-2(1H)-yl)methyl)-4-ethoxynicotinonitrile;
4-ethoxy-6-((7-(2-(ethyl(methyl)amino)ethyl)-5-(6-fluoro-2-methylpyridin-3-yl)-1-oxo-3,4-dihydroisoquinolin-2(1H)-yl)methyl)nicotinonitrile;
(S)-6-(1-(5-(1,4-dimethyl-6-oxo-1,6-dihydropyridin-3-yl)-7-(2-(ethyl(methyl)amino)ethyl)-1-oxo-3,4-dihydroisoquinolin-2(1H)-yl)ethyl)-4 ethoxynicotinonitrile;
2-((4-ethoxy-5-fluoropyridin-2-yl)methyl)-7-(2-(ethyl(methyl)amino)ethyl)-5-(6-fluoro-2-methylpyridin-3-yl)-3,4-dihydroisoquinolin-1(2H)-one;
6-((5-(5-chloro-2-methylpyridin-4-yl)-7-(2-(ethyl(methyl)amino)ethyl)-1-oxo-3,4-dihydroisoquinolin-2(1H)-yl)methyl)-4-ethoxynicotinonitrile;
(S)-6-(1-(5-(1,3-dimethyl-1H-pyrazol-4-yl)-7-(2-(ethyl(methyl)amino)ethyl)-1-oxo-3,4-dihydroisoquinolin-2(1H)-yl)ethyl)-4-ethoxynicotinonitrile;
6-((5-(1,4-dimethyl-6-oxo-1,6-dihydropyridin-3-yl)-7-(2-(ethyl(methyl)amino)ethyl)-1-oxo-3,4-dihydroisoquinolin-2(1H)-yl)methyl)-4-ethoxynicotinonitrile;
6-(1-(5-(5-chloro-2-methoxypyridin-4-yl)-7-(2-(ethyl(methyl)amino)ethyl)-1-oxo-3,4-dihydroisoquinolin-2(1H)-yl)ethyl)-4-ethoxynicotinonitrile;
7-(2-(ethyl(methyl)amino)ethyl)-5-(6-fluoro-2-methylpyridin-3-yl)-2-((6-fluoro-5-methoxypyridin-3-yl)methyl)-3,4-dihydroisoquinolin-1(2H)-one;
(S)-6-(1-(5-(5-amino-2-(trifluoromethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(2-methylpyridin-3-yl)-1-oxo-3,4-dihydroisoquinolin-2(1H)-yl)ethyl)nicotinonitrile;
6-((5-(2,5-dimethylpyridin-3-yl)-7-(2-(ethyl(methyl)amino)ethyl)-1-oxo-3,4-dihydroisoquinolin-2(1H)-yl)methyl)-4-ethoxynicotinonitrile;
4-ethoxy-6-(1-(7-(2-(ethyl(methyl)amino)ethyl)-5-(2-fluoro-3-methylpyridin-4-yl)-1-oxo-3,4-dihydroisoquinolin-2(1H)-yl)ethyl)nicotinonitrile;
(S)-4-ethoxy-6-(1-(7-(2-(ethyl(methyl)amino)ethyl)-5-(5-fluoro-2-methylpyridin-4-yl)-1-oxo-3,4-dihydroisoquinolin-2(1H)-yl)ethyl)nicotinonitrile;
4-ethoxy-6-(1-(7-(2-(ethyl(methyl)amino)ethyl)-5-(2-fluoro-5-methylpyridin-4-yl)-1-oxo-3,4-dihydroisoquinolin-2(1H)-yl)ethyl)nicotinonitrile;
6-((5-(2-(difluoromethyl)-5-methylpyridin-3-yl)-7-(2-(ethyl(methyl)amino)ethyl)-1-oxo-3,4-dihydroisoquinolin-2(1H)-yl)methyl)-4-ethoxynicotinonitrile;
(S)-5-(1,3-dimethyl-1H-pyrazol-4-yl)-2-(1-(4-ethoxy-5-fluoropyridin-2-yl)ethyl)-7-(2-(ethyl(methyl)amino)ethyl)-3,4-dihydroisoquinolin-1(2H)-one;
5-(3-(difluoromethyl)-1-methyl-1H-pyrazol-4-yl)-7-(2-(ethyl(methyl)amino)ethyl)-2-(1-(6-fluoro-5-methoxypyridin-3-yl)ethyl)-3,4-dihydroisoquinolin-1(2H)-one;
6-(1-(5-(6-chloro-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-(1-(7-(2-(ethyl(methyl)amino)ethyl)-5-(1-methyl-3-(trifluoromethyl)-1H-pyrazol-4-yl)-1-oxo-3,4-dihydroisoquinolin-2(1H)-yl)ethyl)nicotinonitrile;
(S)-6-(1-(5-(4-(difluoromethyl)-1-methyl-6-oxo-1,6-dihydropyridin-3-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-(1-(7-(2-(ethyl(methyl)amino)ethyl)-5-(5-hydroxy-2-methylpyridin-3-yl)-1-oxo-3,4-dihydroisoquinolin-2(1H)-yl)ethyl)nicotinonitrile;
4-ethoxy-6-(1-(7-(2-(ethyl(methyl)amino)ethyl)-5-(2-ethylpyridin-3-yl)-1-oxo-3,4-dihydroisoquinolin-2(1H)-yl)ethyl)nicotinonitrile;
(S)-6-(1-(5-(3-chloropyridin-4-yl)-7-(2-(ethyl(methyl)amino)ethyl)-1-oxo-3,4-dihydroisoquinolin-2(1H)-yl)ethyl)-4-ethoxynicotinonitrile;
6-(1-(5-(2-chloro-5-(1-hydroxyethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-((7-(2-(ethyl(methyl)amino)ethyl)-5-(5-fluoro-2-methylpyridin-4-yl)-1-oxo-3,4-dihydroisoquinolin-2(1H)-yl)methyl)nicotinonitrile;
6-((5-(6-amino-2-methylpyridin-3-yl)-7-(2-(ethyl(methyl)amino)ethyl)-1-oxo-3,4-dihydroisoquinolin-2(1H)-yl)methyl)-4-ethoxynicotinonitrile;
(R)-6-(1-(5-(2,5-dimethylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(1-methyl-1H-imidazol-5-yl)-1-oxo-3,4-dihydroisoquinolin-2(1H)-yl)ethyl)nicotinonitrile;
6-(1-(5-cyclopropyl-7-(2-(ethyl(methyl)amino)ethyl)-1-oxo-3,4-dihydroisoquinolin-2(1H)-yl)ethyl)-4-ethoxynicotinonitrile;
4-ethoxy-6-(1-(7-(2-(ethyl(methyl)amino)ethyl)-1-oxo-5-(1,2,3,6-tetrahydropyridin-4-yl)-3,4-dihydroisoquinolin-2(1H)-yl)ethyl)nicotinonitrile;
(S)-6-(1-(5-(5-cyclopropyl-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(1,3-dimethyl-2-oxo-1,2-dihydropyridin-4-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(2-amino-5-methylpyridin-4-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(2,5-dimethylpyridin-4-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-((7-(2-(ethyl(methyl)amino)ethyl)-5-(2-methoxy-5-methylpyridin-4-yl)-1-oxo-3,4-dihydroisoquinolin-2(1H)-yl)methyl)nicotinonitrile;
(S)-6-(1-(5-(3-(difluoromethyl)-1-ethyl-1H-pyrazol-4-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(5-chloro-2-methylpyridin-4-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(5-methoxy-2-methylpyridin-3-yl)-1-oxo-3,4-dihydroisoquinolin-2(1H)-yl)ethyl)nicotinonitrile;
6-(1-(5-(3,6-dichloropyridazin-4-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(5-fluoro-1,4-dimethyl-6-oxo-1,6-dihydropyridin-3-yl)-1-oxo-3,4-dihydroisoquinolin-2(1H)-yl)ethyl)nicotinonitrile;
(S)-6-(1-(5-(5-chloro-1,4-dimethyl-6-oxo-1,6-dihydropyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(1-ethyl-4-methyl-6-oxo-1,6-dihydropyridin-3-yl)-1-oxo-3,4-dihydroisoquinolin-2(1H)-yl)ethyl)nicotinonitrile;
(S)-6-(1-(5-(5-(difluoromethyl)-2-methylpyridin-4-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(3,6-dimethylpyridazin-4-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(4-chloro-1-(difluoromethyl)-6-oxo-1,6-dihydropyridin-3-yl)-7-(2-(ethyl(methyl)amino)ethyl)-1-oxo-3,4-dihydroisoquinolin-2(1H)-yl)ethyl)-4 ethoxynicotinonitrile;
(S)-6-(1-(5-(4-chloro-1-methyl-6-oxo-1,6-dihydropyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-1-oxo-5-(6-oxo-4-(trifluoromethyl)-1,6-dihydropyridin-3-yl)-3,4-dihydroisoquinolin-2(1H)-yl)ethyl)nicotinonitrile;
(S)-6-(1-(5-(5-(difluoromethyl)-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(6-amino-2-chloropyridin-3-yl)-7-(2-(ethyl(methyl)amino)ethyl)-1-oxo-3,4-dihydroisoquinolin-2(1H)-yl)ethyl)-4-ethoxynicotinonitrile;
6-((5-(2,5-dimethylpyridin-4-yl)-7-(2-(ethyl(methyl)amino)ethyl)-1-oxo-3,4-dihydroisoquinolin-2(1H)-yl)methyl)-4-ethoxynicotinonitrile;
6-((5-(2-amino-5-methylpyridin-4-yl)-7-(2-(ethyl(methyl)amino)ethyl)-1-oxo-3,4-dihydroisoquinolin-2(1H)-yl)methyl)-4-ethoxynicotinonitrile;
(S)-4-ethoxy-6-(1-(7-(2-(ethyl(methyl)amino)ethyl)-5-(5-methyloxazolo[4,5-b]pyridin-6-yl)-1-oxo-3,4-dihydroisoquinolin-2(1H)-yl)ethyl)nicotinonitrile;
(S)-4-ethoxy-6-(1-(7-(2-(ethyl(methyl)amino)ethyl)-5-(1-methyl-6-oxo-4-(trifluoromethyl)-1,6-dihydropyridin-3-yl)-1-oxo-3,4-dihydroisoquinolin-2(1H)-yl)ethyl)nicotinonitrile;
(S)-6-(1-(5-(2-amino-5-chloropyridin-4-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(2-(2-hydroxypropan-2-yl)-5-methylpyridin-4-yl)-1-oxo-3,4-dihydroisoquinolin-2(1H)-yl)ethyl)nicotinonitrile;
(S)-6-(1-(5-(5-acetyl-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-((1S)-1-(7-(2-(ethyl(methyl)amino)ethyl)-5-(5-(1-hydroxyethyl)-2-methylpyridin-3-yl)-1-oxo-3,4-dihydroisoquinolin-2(1H)-yl)ethyl)nicotinonitrile;
(S)-6-(1-(5-(5-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(2-methoxy-5-methylpyridin-4-yl)-1-oxo-3,4-dihydroisoquinolin-2(1H)-yl)ethyl)nicotinonitrile;
6-((5-(3,6-dimethylpyridazin-4-yl)-7-(2-(ethyl(methyl)amino)ethyl)-1-oxo-3,4-dihydroisoquinolin-2(1H)-yl)methyl)-4-ethoxynicotinonitrile;
(S)-4-ethoxy-6-(1-(7-(2-(ethyl(methyl)amino)ethyl)-5-(7-methylimidazo[1,2-a]pyridin-6-yl)-1-oxo-3,4-dihydroisoquinolin-2(1H)-yl)ethyl)nicotinonitrile;
4-ethoxy-6-((1S)-1-(7-(2-(ethyl(methyl)amino)ethyl)-5-(4-methoxy-2-methylpyridin-3-yl)-1-oxo-3,4-dihydroisoquinolin-2(1H)-yl)ethyl)nicotinonitrile;
(S)-6-(1-(5-(5-(cyanomethyl)-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(4-ethyl-1-methyl-6-oxo-1,6-dihydropyridin-3-yl)-1-oxo-3,4-dihydroisoquinolin-2(1H)-yl)ethyl)nicotinonitrile;
(S)-6-(1-(5-(3-chloro-1-methyl-2-oxo-1,2-dihydropyridin-4-yl)-7-(2-(ethyl(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(ethyl(methyl)amino)ethyl)-5-(1-methyl-2-oxo-3-(trifluoromethyl)-1,2-dihydropyridin-4-yl)-1-oxo-3,4-dihydroisoquinolin-2(1H)-yl)ethyl)nicotinonitrile;
4-ethoxy-6-((7-(2-(ethyl(methyl)amino)ethyl)-5-(1-methyl-6-oxo-4-(trifluoromethyl)-1,6-dihydropyridin-3-yl)-1-oxo-3,4-dihydroisoquinolin-2(1H)-yl)methyl)nicotinonitrile;
6-(1-(5-(5-chloro-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(S)-6-(1-(5-(6-amino-2-(trifluoromethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
(R)-6-(1-(5-(6-amino-2-(trifluoromethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
6-(1-(5-(6-amino-2-(difluoromethyl)pyridin-3-yl)-7-(2-(ethyl(methyl)amino)ethyl)-1-oxo-3,4-dihydroisoquinolin-2(1H)-yl)ethyl)-4-ethoxynicotinonitrile;
6-((5-(5-amino-2-methylpyridin-3-yl)-7-(2-(ethyl(methyl)amino)ethyl)-1-oxo-3,4-dihydroisoquinolin-2(1H)-yl)methyl)-4-ethoxynicotinonitrile;
(S)-6-(1-(5-(2-amino-5-(trifluoromethyl)pyridin-4-yl)-7-(2-(ethyl(methyl)amino)ethyl)-1-oxo-3,4-dihydroisoquinolin-2(1H)-yl)ethyl)-4-ethoxynicotinonitrile;
6-(1-(5-(6-chloro-3-methylpyridazin-4-yl)-7-(2-(ethyl(methyl)amino)ethyl)-1-oxo-3,4-dihydroisoquinolin-2(1H)-yl)ethyl)-4-ethoxynicotinonitrile;
(S)-2-(1-(4-ethoxy-5-fluoropyridin-2-yl)ethyl)-7-(2-(ethylmethyl)amino)ethyl)-5-(1-methyl-6-oxo-4-(trifluoromethyl)-1,6-dihydropyridin-3-yl)-3,4-dihydroisoquinolin-1(2H)-one;
6-(1-(5-(1,3-dimethyl-1H-pyrazol-5-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-(1-(7-(2-(ethyl(methyl)amino)ethyl)-5-(5-methyl-1H-imidazo[4,5-b]pyridin-6-yl)-1-oxo-3,4-dihydroisoquinolin-2(1H)-yl)ethyl)nicotinonitrile;
4-ethoxy-6-(1-(7-(2-(ethyl(methyl)amino)ethyl)-1-oxo-5-(pyrrolidin-1-yl)-3,4-dihydroisoquinolin-2(1H)-yl)ethyl)nicotinonitrile;
4-ethoxy-6-(1-(7-(2-(ethyl(methyl)amino)ethyl)-5-(1H-imidazol-1-yl)-1-oxo-3,4-dihydroisoquinolin-2(1H)-yl)ethyl)nicotinonitrile;
4-ethoxy-6-(1-(7-(2-(ethyl(methyl)amino)ethyl)-1-oxo-5-(1H-pyrazol-1-yl)-3,4-dihydroisoquinolin-2(1H)-yl)ethyl)nicotinonitrile;
4-ethoxy-6-((S)-1-(7-(2-(ethyl(methyl)amino)ethyl)-5-((S)-3-hydroxypyrrolidin-1-yl)-1-oxo-3,4-dihydroisoquinolin-2(1H)-yl)ethyl)nicotinonitrile;
5-(3,6-dimethylpyridazin-4-yl)-2-(1-(4-ethoxy-5-methylpyridin-2-yl)ethyl)-7-(2-(ethyl(methyl)amino)ethyl)-3,4-dihydroisoquinolin-1(2H)-one;
5-(3,6-dimethylpyridazin-4-yl)-2-((4-ethoxy-5-methylpyridin-2-yl)methyl)-7-(2-(ethyl(methyl)amino)ethyl)-3,4-dihydroisoquinolin-1(2H)-one;
6-((S)-1-(5-(3-(difluoromethyl)-1-methyl-1H-pyrazol-4-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
6-((S)-1-(5-(5-amino-2-(trifluoromethyl)pyridin-3-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
6-((S)-1-(5-(4-chloro-1-methyl-6-oxo-1,6-dihydropyridin-3-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
6-((S)-1-(5-(1,4-dimethyl-6-oxo-1,6-dihydropyridin-3-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
4-ethoxy-6-((S)-1-(5-(6-fluoro-2-methylpyridin-3-yl)-7-(2-((2R,3R)-3-hydroxy-2-methylazetidin-1-yl)ethyl)-1-oxo-3,4-dihydroisoquinolin-2(1H)-yl)ethyl)nicotinonitrile;
6-((S)-1-(5-(5-(difluoromethyl)-2-methylpyridin-3-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
6-((S)-1-(5-(6-amino-2-methylpyridin-3-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
6-((S)-1-(5-(2,5-dimethylpyridin-3-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
6-((S)-1-(5-(2,5-dimethylpyridin-4-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
4-ethoxy-6-((5-(6-fluoro-2-methylpyridin-3-yl)-7-(2-((2R,3R)-3-hydroxy-2-methylazetidin-1-yl)ethyl)-1-oxo-3,4-dihydroisoquinolin-2(1H)-yl)methyl)nicotinonitrile;
6-((R)-1-(5-(2,5-dimethylpyridin-4-yl)-7-(2-((2R,3R)-3-hydroxy-2-methylazetidin-1-yl)ethyl)-1-oxo-3,4-dihydroisoquinolin-2(1H)-yl)ethyl)-4-ethoxynicotinonitrile;
4-ethoxy-6-((7-(2-((2R,3R)-3-hydroxy-2-methylazetidin-1-yl)ethyl)-5-(1-methyl-6-oxo-4-(trifluoromethyl)-1,6-dihydropyridin-3-yl)-1-oxo-3,4-dihydroisoquinolin-2(1H)-yl)methyl)nicotinonitrile;
4-ethoxy-6-((S)-1-(5-(6-fluoro-2-methylpyridin-3-yl)-7-(2-((2R, 3R)-3-hydroxy-2-methylpyrrolidin-1-yl)ethyl)-1-oxo-3,4-dihydroisoquinolin-2(1H)-yl)ethyl)nicotinonitrile;
4-ethoxy-6-((S)-1-(5-(6-fluoro-2-methylpyridin-3-yl)-7-(2-((2R,4S)-4-hydroxy-2-methylpyrrolidin-1-yl)ethyl)-1-oxo-3,4-dihydroisoquinolin-2(1H)-yl)ethyl)nicotinonitrile;
4-ethoxy-6-((S)-1-(5-(6-fluoro-2-methylpyridin-3-yl)-7-(2-((2S,4S)-4-hydroxy-2-methylpyrrolidin-1-yl)ethyl)-1-oxo-3,4-dihydroisoquinolin-2(1H)-yl)ethyl)nicotinonitrile;
(S)-4-ethoxy-6-(1-(5-(5-fluoro-2-(trifluoromethyl)pyridin-3-yl)-7-(2-((2-hydroxyethyl)(methyl)amino)ethyl)-1-oxo-3,4-dihydroisoquinolin-2(1H)-yl)ethyl)nicotinonitrile;
(S)-6-(1-(7-(2-(cyclopropyl(methyl)amino)ethyl)-5-(6-fluoro-2-methylpyridin-3-yl)-1-oxo-3,4-dihydroisoquinolin-2(1H)-yl)ethyl)-4-ethoxynicotinonitrile;
(S)-6-(1-(5-(5-amino-2-(trifluoromethyl)pyridin-3-yl)-7-(2-((2-hydroxyethyl)(methyl)amino)ethyl)-1-oxo-3,4-dihydroisoquinolin-2(1H)-yl)ethyl)-4-ethoxynicotinonitrile;
4-ethoxy-6-(1-(5-(6-fluoro-2-methylpyridin-3-yl)-7-(2-(methyl(oxetan-3-yl)amino)ethyl)-1-oxo-3,4-dihydroisoquinolin-2(1H)-yl)ethyl)nicotinonitrile;
(S)-6-(1-(5-(2,5-dimethylpyridin-4-yl)-7-(2-((2-methoxyethyl)(methyl)amino)ethyl)-1-oxo-3,4-dihydroisoquinolin-2(1H)-yl)ethyl)-4-ethoxynicotinonitrile;
6-(1-(5-(4-chloro-1-methyl-6-oxo-1,6-dihydropyridin-3-yl)-7-(2-((2-methoxyethyl)(methyl)amino)ethyl)-1-oxo-3,4-dihydroisoquinolin-2(1H)-yl)ethyl)-4-ethoxynicotinonitrile;
(S)-4-ethoxy-6-(1-(5-(6-fluoro-2-methylpyridin-3-yl)-7-(2-((2-methoxyethyl)(methyl)amino)ethyl)-1-oxo-3,4-dihydroisoquinolin-2(1H)-yl)ethyl)nicotinonitrile;
4-ethoxy-6-((S)-1-(5-(6-fluoro-2-methylpyridin-3-yl)-7-(2-((R)-2-methylazetidin-1-yl)ethyl)-1-oxo-3,4-dihydroisoquinolin-2(1H)-yl)ethyl)nicotinonitrile;
4-ethoxy-6-(1-(5-(6-fluoro-2-methylpyridin-3-yl)-7-(2-((2-fluoroethyl)(methyl)amino)ethyl)-1-oxo-3,4-dihydroisoquinolin-2(1H)-yl)ethyl)nicotinonitrile;
6-(1-(5-(4-chloro-1-methyl-6-oxo-1,6-dihydropyridin-3-yl)-7-(2-((2-fluoroethyl)(methyl)amino)ethyl)-1-oxo-3,4-dihydroisoquinolin-2(1H)-yl)ethyl)-4-ethoxynicotinonitrile;
(S)-6-(1-(5-(4-chloro-1-methyl-6-oxo-1,6-dihydropyridin-3-yl)-7-(2-((2-hydroxyethyl)(methyl)amino)ethyl)-1-oxo-3,4-dihydroisoquinolin-2(1H)-yl)ethyl)-4-ethoxynicotinonitrile;
6-((1S)-1-(5-(4-chloro-1-methyl-6-oxo-1,6-dihydropyridin-3-yl)-7-(2-((2-hydroxypropyl)(methyl)amino)ethyl)-1-oxo-3,4-dihydroisoquinolin-2(1H)-yl)ethyl)-4-ethoxynicotinonitrile;
(S)-6-(1-(5-(4-chloro-1-methyl-6-oxo-1,6-dihydropyridin-3-yl)-7-(2-((2-methoxyethyl)(methyl)amino)ethyl)-1-oxo-3,4-dihydroisoquinolin-2(1H)-yl)ethyl)-4-ethoxynicotinonitrile;
6-(1-(5-(1,3-dimethyl-1H-pyrazol-4-yl)-7-(2-((2-fluoroethyl)(methyl)amino)ethyl)-1-oxo-3,4-dihydroisoquinolin-2(1H)-yl)ethyl)-4-ethoxynicotinonitrile;
4-ethoxy-6-((1S)-1-(5-(6-fluoro-2-methylpyridin-3-yl)-7-(2-((2-hydroxypropyl)(methyl)amino)ethyl)-1-oxo-3,4-dihydroisoquinolin-2(1H)-yl)ethyl)nicotinonitrile;
4-ethoxy-6-((S)-1-(5-(6-fluoro-2-methylpyridin-3-yl)-7-(2-((S)-2-(hydroxymethyl)azetidin-1-yl)ethyl)-1-oxo-3,4-dihydroisoquinolin-2(1H)-yl)ethyl)nicotinonitrile;
4-ethoxy-6-((S)-1-(5-(6-fluoro-2-methylpyridin-3-yl)-7-(2-((2R,3R)-3-methoxy-2-methylazetidin-1-yl)ethyl)-1-oxo-3,4-dihydroisoquinolin-2(1H)-yl)ethyl)nicotinonitrile;
(R)-4-ethoxy-6-((5-(6-fluoro-2-methylpyridin-3-yl)-7-(2-(2-methylazetidin-1-yl)ethyl)-1-oxo-3,4-dihydroisoquinolin-2(1H)-yl)methyl)nicotinonitrile;
6-((1S)-1-(5-(2,5-dimethylpyridin-3-yl)-7-(2-((2-hydroxypropyl)(methyl)amino)ethyl)-1-oxo-3,4-dihydroisoquinolin-2(1H)-yl)ethyl)-4-ethoxynicotinonitrile;
(S)-4-ethoxy-6-(1-(5-(6-fluoro-2-methylpyridin-3-yl)-7-(2-((2-fluoroethyl)(methyl)amino)ethyl)-1-oxo-3,4-dihydroisoquinolin-2(1H)-yl)ethyl)nicotinonitrile;
6-(1-(5-(2,5-dimethylpyridin-4-yl)-7-(2-((2-fluoroethyl)(methyl)amino)ethyl)-1-oxo-3,4-dihydroisoquinolin-2(1H)-yl)ethyl)-4-ethoxynicotinonitrile;
(S)-4-ethoxy-6-(1-(7-(2-(3-fluoroazetidin-1-yl)ethyl)-5-(1-methyl-6-oxo-4-(trifluoromethyl)-1,6-dihydropyridin-3-yl)-1-oxo-3,4-dihydroisoquinolin-2(1H)-yl)ethyl)nicotinonitrile;
(S)-6-(1-(5-(2,5-dimethylpyridin-4-yl)-7-(2-((2-hydroxyethyl)(methyl)amino)ethyl)-1-oxo-3,4-dihydroisoquinolin-2(1H)-yl)ethyl)-4-ethoxynicotinonitrile;
(S)-6-(1-(5-(4-chloro-1-methyl-6-oxo-1,6-dihydropyridin-3-yl)-7-(2-((2-fluoroethyl)(methyl)amino)ethyl)-1-oxo-3,4-dihydroisoquinolin-2(1H)-yl)ethyl)-4-ethoxynicotinonitrile;
(S)-4-ethoxy-6-(1-(5-(6-fluoro-2-methylpyridin-3-yl)-7-(2-((2-hydroxyethyl)(methyl)amino)ethyl)-1-oxo-3,4-dihydroisoquinolin-2(1H)-yl)ethyl)nicotinonitrile;
(S)-4-ethoxy-6-(1-(5-(6-fluoro-2-methylpyridin-3-yl)-7-(2-(methyl(oxetan-3-yl)amino)ethyl)-1-oxo-3,4-dihydroisoquinolin-2(1H)-yl)ethyl)nicotinonitrile;
(S)-6-(1-(5-(2,5-dimethylpyridin-4-yl)-7-(2-((2-fluoroethyl)(methyl)amino)ethyl)-1-oxo-3,4-dihydroisoquinolin-2(1H)-yl)ethyl)-4-ethoxynicotinonitrile;
7-(2-(dimethylamino)ethyl)-2-(4-fluoro-3-methoxybenzyl)-5-(1-methyl-3-(trifluoromethyl)-1H-pyrazol-4-yl)-3,4-dihydroisoquinolin-1(2H)-one;
4-ethoxy-6-((1S)-1-(7-(2-(ethyl(methyl)amino)ethyl)-5-(1-hydroxy-2-methylpropyl)-1-oxo-3,4-dihydroisoquinolin-2(1H)-yl)ethyl)nicotinonitrile;
4-ethoxy-6-(1-(7-(2-(ethyl(methyl)amino)ethyl)-5-(1-hydroxy-2,2-dimethylpropyl)-1-oxo-3,4-dihydroisoquinolin-2(1H)-yl)ethyl)nicotinonitrile;
4-ethoxy-6-(1-(7-(2-(ethyl(methyl)amino)ethyl)-1-oxo-5-(3,3,3-trifluoro-1-hydroxy-2-methylpropyl)-3,4-dihydroisoquinolin-2(1H)-yl)ethyl)nicotinonitrile;
4-ethoxy-6-(1-(7-(2-(ethyl(methyl)amino)ethyl)-1-oxo-5-(2,2,2-trifluoro-1-hydroxyethyl)-3,4-dihydroisoquinolin-2(1H)-yl)ethyl)nicotinonitrile;
2-(1-(4-ethoxy-5-fluoropyridin-2-yl)ethyl)-7-(2-(ethyl(methyl)amino)ethyl)-5-(2,2,2-trifluoro-1-hydroxyethyl)-3,4-dihydroisoquinolin-1(2H)-one;
(R)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
(R)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
6-((5-(1,3-dimethyl-1H-pyrazol-4-yl)-7-((2-methyl-1H-imidazol-1-yl)methyl)-1-oxo-3,4-dihydroisoquinolin-2(1H)-yl)methyl)-4-ethoxynicotinonitrile;
(S)-6-(1-(5-(1,3-dimethyl-1H-1,2,4-triazol-5-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(3,5-dimethyl-1H-1,2,4-triazol-1-yl)-7-((2-methyl-1H-imidazol-1-yl)methyl)-1-oxo-3,4-dihydroisoquinolin-2(1H)-yl)ethyl)-4-ethoxynicotinonitrile;
4-ethoxy-6-((S)-1-(7-((2-methyl-1H-imidazol-1-yl)methyl)-5-((S)-1-methylpyrrolidin-2-yl)-1-oxo-3,4-dihydroisoquinolin-2(1H)-yl)ethyl)nicotinonitrile;
4-ethoxy-6-((S)-1-(7-((2-methyl-1H-imidazol-1-yl)methyl)-5-((R)-1-methylpyrrolidin-2-yl)-1-oxo-3,4-dihydroisoquinolin-2(1H)-yl)ethyl)nicotinonitrile;
(S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinamide;
(S)-4-ethoxy-6-(1-(7-((2-methyl-1H-imidazol-1-yl)methyl)-1-oxo-5-(6-oxo-4-(trifluoromethyl)-1,6-dihydropyridin-3-yl)-3,4-dihydroisoquinolin-2(1H)-yl)ethyl)nicotinonitrile;
(S)-6-(1-(5-(3-((dimethylamino)methyl)-1-methyl-1H-pyrazol-5-yl)-7-((2-methyl-1H-imidazol-1-yl)methyl)-1-oxo-3,4-dihydroisoquinolin-2(1H)-yl)ethyl)-4-ethoxynicotinonitrile;
(S)-4-ethoxy-6-(1-(5-(1-(2-hydroxyethyl)-3-methyl-1H-pyrazol-4-yl)-7-((2-methyl-1H-imidazol-1-yl)methyl)-1-oxo-3,4-dihydroisoquinolin-2(1H)-yl)ethyl)nicotinonitrile;
(S)-6-(1-(7-((2,4-dimethyl-1H-imidazol-1-yl)methyl)-5-(1-(2-hydroxyethyl)-3-methyl-1H-pyrazol-4-yl)-1-oxo-3,4-dihydroisoquinolin-2(1H)-yl)ethyl)-4-ethoxynicotinonitrile;
(S)-6-(1-(7-((2,4-dimethyl-1H-imidazol-1-yl)methyl)-5-(1,3-dimethyl-1H-pyrazol-4-yl)-1-oxo-3,4-dihydroisoquinolin-2(1H)-yl)ethyl)-4-ethoxynicotinonitrile;
(S)-6-(1-(7-((2,4-dimethyl-1H-imidazol-1-yl)methyl)-5-(1,4-dimethyl-6-oxo-1,6-dihydropyridin-3-yl)-1-oxo-3,4-dihydroisoquinolin-2(1H)-yl)ethyl)-4-ethoxynicotinonitrile;
(S)-6-(1-(5-(1,4-dimethyl-6-oxo-1,6-dihydropyridin-3-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(1,3-dimethyl-1H-pyrazol-4-yl)-7-((4-methyl-1H-imidazol-1-yl)methyl)-1-oxo-3,4-dihydroisoquinolin-2(1H)-yl)ethyl)-4-ethoxynicotinonitrile;
(S)-6-(1-(5-(1,4-dimethyl-1H-pyrazol-3-yl)-7-((2-methyl-1H-imidazol-1-yl)methyl)-1-oxo-3,4-dihydroisoquinolin-2(1H)-yl)ethyl)-4-ethoxynicotinonitrile;
(S)-2-(1-(4-ethoxy-5-fluoropyridin-2-yl)ethyl)-7-((2-methyl-1H-imidazol-1-yl)methyl)-5-(1-methyl-6-oxo-4-(trifluoromethyl)-1,6-dihydropyridin-3-yl)-3,4-dihydroisoquinolin-1(2H)-one;
(S)-4-ethoxy-6-(1-(7-((2-methyl-1H-imidazol-1-yl)methyl)-5-(1-methyl-1H-pyrazol-5-yl)-1-oxo-3,4-dihydroisoquinolin-2(1H)-yl)ethyl)nicotinonitrile;
(S)-6-(1-(5-(1,3-dimethyl-1H-pyrazol-5-yl)-7-((2-methyl-1H-imidazol-1-yl)methyl)-1-oxo-3,4-dihydroisoquinolin-2(1H)-yl)ethyl)-4-ethoxynicotinonitrile;
(S)-5-(1,4-dimethyl-6-oxo-1,6-dihydropyridin-3-yl)-2-(1-(4-ethoxy-5-fluoropyridin-2-yl)ethyl)-7-((2-methyl-1H-imidazol-1-yl)methyl)-3,4-dihydroisoquinolin-1(2H)-one;
(S)-6-(1-(5-(5-(difluoromethyl)-2-methylpyridin-3-yl)-7-((2-methyl-1H-imidazol-1-yl)methyl)-1-oxo-3,4-dihydroisoquinolin-2(1H)-yl)ethyl)-4-ethoxynicotinonitrile;
(S)-2-(1-(4-ethoxy-5-fluoropyridin-2-yl)ethyl)-5-(5-fluoro-1,4-dimethyl-6-oxo-1,6-dihydropyridin-3-yl)-7-((2-methyl-1H-imidazol-1-yl)methyl)-3,4-dihydroisoquinolin-1(2H)-one;
(R)-4-ethoxy-6-(1-(5-(3-methyl-1-(methylsulfonyl)-1H-pyrazol-4-yl)-7-((2-methyl-1H-imidazol-1-yl)methyl)-1-oxo-3,4-dihydroisoquinolin-2(1H)-yl)ethyl)nicotinonitrile;
(S)-6-(1-(5-(4-(difluoromethyl)-1-methyl-6-oxo-1,6-dihydropyridin-3-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(1-(difluoromethyl)-3-methyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
(S)-4-ethoxy-6-(1-(5-(5-fluoro-1,4-dimethyl-6-oxo-1,6-dihydropyridin-3-yl)-7-((2-methyl-1H-imidazol-1-yl)methyl)-1-oxo-3,4-dihydroisoquinolin-2(1H)-yl)ethyl)nicotinonitrile;
(S)-4-ethoxy-6-(1-(7-((2-methyl-1H-imidazol-1-yl)methyl)-5-(1-methyl-6-oxo-4-(trifluoromethyl)-1,6-dihydropyridin-3-yl)-1-oxo-3,4-dihydroisoquinolin-2(1H)-yl)ethyl)nicotinonitrile;
(S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(2,5-dimethyl-2H-1,2,3-triazol-4-yl)-7-((2-methyl-1H-imidazol-1-yl)methyl)-1-oxo-3,4-dihydroisoquinolin-2(1H)-yl)ethyl)-4-ethoxynicotinonitrile;
(S)-4-ethoxy-6-(1-(7-((2-methyl-1H-imidazol-1-yl)methyl)-5-(1-methyl-6-oxo-4-(trifluoromethyl)-1,6-dihydropyridazin-3-yl)-1-oxo-3,4-dihydroisoquinolin-2(1H)-yl)ethyl)nicotinonitrile;
(S)-6-(1-(5-(4-chloro-1-methyl-6-oxo-1,6-dihydropyridin-3-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(3-(difluoromethyl)-1-methyl-1H-1,2,4-triazol-5-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-(5-(difluoromethyl)-3-methyl-1H-1,2,4-triazol-1-yl)-7-((2-methyl-1H-imidazol-1-yl)methyl)-1-oxo-3,4-dihydroisoquinolin-2(1H)-yl)ethyl)-4-ethoxynicotinonitrile;
(S)-6-(1-(5-((dimethylamino)methyl)-7-((2-methyl-1H-imidazol-1-yl)methyl)-1-oxo-3,4-dihydroisoquinolin-2(1H)-yl)ethyl)-4-ethoxynicotinonitrile;
(S)-4-methoxy-6-(1-(7-((2-methyl-1H-imidazol-1-yl)methyl)-5-(1-methyl-6-oxo-4-(trifluoromethyl)-1,6-dihydropyridazin-3-yl)-1-oxo-3,4-dihydroisoquinolin-2(1H)-yl)ethyl)nicotinonitrile;
4-ethoxy-6-((7-((2-methyl-1H-imidazol-1-yl)methyl)-5-(1-methyl-6-oxo-4-(trifluoromethyl)-1,6-dihydropyridin-3-yl)-1-oxo-3,4-dihydroisoquinolin-2(1H)-yl)methyl)nicotinonitrile;
6-((5-(5-(difluoromethyl)-3-methyl-1H-1,2,4-triazol-1-yl)-7-((2-methyl-1H-imidazol-1-yl)methyl)-1-oxo-3,4-dihydroisoquinolin-2(1H)-yl)methyl)-4-ethoxynicotinonitrile;
6-((5-(4-(difluoromethyl)-1-methyl-6-oxo-1,6-dihydropyridin-3-yl)-7-((2-methyl-1H-imidazol-1-yl)methyl)-1-oxo-3,4-dihydroisoquinolin-2(1H)-yl)methyl)-4-ethoxynicotinonitrile;
6-((5-(1,3-dimethyl-1H-pyrazol-5-yl)-7-((2-methyl-1H-imidazol-1-yl)methyl)-1-oxo-3,4-dihydroisoquinolin-2(1H)-yl)methyl)-4-ethoxynicotinonitrile;
4-ethoxy-6-((5-(3-methyl-1-(methylsulfonyl)-1H-pyrazol-4-yl)-7-((2-methyl-1H-imidazol-1-yl)methyl)-1-oxo-3,4-dihydroisoquinolin-2(1H)-yl)methyl)nicotinonitrile;
(S)-4-ethoxy-6-(1-(7-((2-methyl-1H-imidazol-1-yl)methyl)-5-(3-methyl-1H-pyrazol-4-yl)-1-oxo-3,4-dihydroisoquinolin-2(1H)-yl)ethyl)nicotinonitrile;
(S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxypyridazine-3-carbonitrile;
4-ethoxy-6-(1-(7-(2-(ethyl(methyl)amino)ethyl)-1-oxo-5-(1,1,1-trifluoro-2-hydroxypropan-2-yl)-3,4-dihydroisoquinolin-2(1H)-yl)ethyl)nicotinonitrile, and
4-ethoxy-6-((7-(2-(ethyl(methyl)amino)ethyl)-1-oxo-5-(2,2,2-trifluoro-1-methoxyethyl)-3,4-dihydroisoquinolin-2(1H)-yl)methyl)nicotinonitrile.

22. The compound of claim 1 selected from:
(S)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(R)-5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
5-((dimethylamino)methyl)-2-(1-(4-ethoxy-5-fluoropyridin-2-yl)ethyl)-7-((2-(methylamino)-1H-imidazol-1-yl)methyl)-3,4-dihydroisoquinolin-1(2H)-one;
(S)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
(R)-2-(1-(6-fluoro-5-methoxypyridin-3-yl)ethyl)-7-((2-(methylamino)-1H-imidazol-1-yl)methyl)-3,4-dihydroisoquinolin-1(2H)-one;
2-(1-(6-fluoro-5-methoxypyridin-3-yl)ethyl)-7-((2-(methylamino)-1H-imidazol-1-yl)methyl)-3,4-dihydroisoquinolin-1(2H)-one;
(S)-4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile;
(R)-4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile;
4-ethoxy-6-(1-(7-(2-(ethyl(methyl)amino)ethyl)-5-(6-fluoro-2-methylpyridin-3-yl)-1-oxo-3,4-dihydroisoquinolin-2(1H)-yl)ethyl)nicotinonitrile;
(S)-6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
(R)-6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
6-(1-(5-(6-amino-2-methylpyridin-3-yl)-7-(2-(ethyl(methyl)amino)ethyl)-1-oxo-3,4-dihydroisoquinolin-2(1H)-yl)ethyl)-4-ethoxynicotinonitrile;
4-ethoxy-6-((7-(2-(ethyl(methyl)amino)ethyl)-5-(1-methyl-6-oxo-4-(trifluoromethyl)-1,6-dihydropyridin-3-yl)-1-oxo-3,4-dihydroisoquinolin-2(1H)-yl)methyl)nicotinonitrile;
(S)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile;
(R)-6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile, and
6-(1-(5-(1,3-dimethyl-1H-pyrazol-4-yl)-7-((2-methyl-1H-imidazol-1-yl)methyl)-1-oxo-3,4-dihydroisoquinolin-2(1H)-yl)ethyl)-4-ethoxynicotinonitrile.

23. A pharmaceutical composition, comprising a compound of claim 1, or a pharmaceutically acceptable salt thereof, and one or more pharmaceutically acceptable carriers.

24. The pharmaceutical composition of claim 23 further comprising one or more additional therapeutic agents.

25. A method for treating a disease mediated by WDR5, wherein the method comprises administering to a subject in need of such treatment a therapeutically effective amount of a compound of claim 1, or a pharmaceutically acceptable salt thereof.

26. (canceled)

27. (canceled)

28. (canceled)

29. The method of claim 25, wherein the disease is a cancer selected from solid tumors, adenoma, bladder cancer, brain cancer, breast cancer, cervical cancer, colorectal cancer, colon cancer, epidermal carcinoma, follicular carcinoma, genitourinary cancers, glioblastoma, head and neck cancers, Hodgkin's disease, non-Hodgkin's lymphoma, hepatoma, kidney cancer, small cell lung cancer, non-small cell lung cancer, acute lymphoblastic leukemia (ALL), acute myelogenous leukemia (AML) or chronic myelogenous leukemia (CML), multiple myeloma, lymphoid disorders, skin cancers including melanoma, neuroblastoma, ovarian cancer, pancreatic cancer, prostate cancer, rectal cancer, sarcoma, testicular cancer, and thyroid cancer.
